(19) United States
(12) Patent Application Publication
(10) Pub. No.: US 2024/0279233 A1
(43) Pub. Date: Aug. 22, 2024

(54) MACROCYCLIC TAK1 INHIBITORS

(71) Applicant: Aqilion AB, Helsingborg (SE)

(72) Inventors: Martin Hans Johansson, Helsingborg (SE); Bo Svensson, Lund (SE); Björn Borgström, Lund (SE); Filip Paulsen, Lund (SE)

(73) Assignee: Aqilion AB, Helsingborg (SE)

(21) Appl. No.: 18/568,249

(22) PCT Filed: Jun. 29, 2022
(86) PCT No.: PCT/EP2022/067990

§ 371 Date: Dec. 7, 2023

(30) Foreign Application Priority Data
Jun. 30, 2021 (GB) 2109446.1
Mar. 10, 2022 (GB) 2203326.0

(51) Int. Cl. C07D 487/04; A61K 31/4188; A61K 31/4192; A61K 31/4196; A61K 31/4245; A61K 31/429; A61K 31/437; A61K 31/4439; A61K 31/454; A61K 31/496; A61K 31/4995; A61K 31/5377; A61K 31/5386; A61K 31/551; C07D 471/14; C07D 495/18; C07D 498/04; C07D 513/18; C07D 519/00

(52) U.S. Cl. CPC C07D 487/04; A61K 31/4188; A61K 31/4192; A61K 31/4196; A61K 31/4245; A61K 31/429; A61K 31/437; A61K 31/4439; A61K 31/454; A61K 31/496; A61K 31/4995; A61K 31/5377; A61K 31/5386; A61K 31/551; C07D 471/14; C07D 495/18; C07D 498/04; C07D 513/18; C07D 519/00

(57) ABSTRACT

The invention provides a compound according to formula (I) or a tautomer thereof, wherein R¹, R², R³, W₁, W₂, W₃, A, X and Y are as defined in the specification. The compounds are potent inhibitors of the serine/threonine kinase, TAK1. The present invention further provides: a pharmaceutical composition comprising a compound of formula (I) and at least one pharmaceutically acceptable excipient; a compound of formula (I) for use as a medicament (for example for the treatment or prophylaxis of a condition selected from cancer, inflammatory diseases, autoinflammatory diseases and autoimmune diseases); and a method of treating or preventing a disease or disorder comprising administering a pharmaceutically effective amount of a compound of formula (I) to a subject in need thereof.

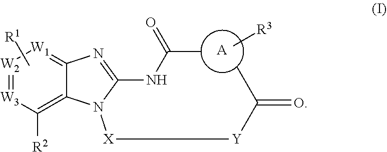

### FIELD OF THE INVENTION

The present invention relates to compounds of formula (I) and compositions comprising compounds of formula (I). The compounds and compositions of the invention are useful in the treatment or prophylaxis of a disease or disorder that may be treated or prevented by inhibition of the serine/threonine kinase, TAK1.

### BACKGROUND OF THE INVENTION

TAK1 (Transforming growth factor beta-activated kinase 1, Mitogen-activated protein kinase kinase kinase 7, MAP3K7) is a serine/threonine kinase belonging to the MAPK kinase kinase (MAP3K) family. TAK1 was originally identified as a kinase involved in TGF-β signaling (Yamaguchi et al., Science, 1995, 270, 2008-2011). It was later shown that TAK1 mediates activation of immune processes stimulated by pro-inflammatory cytokines such as tumor necrosis factor alpha (TNFα), toll-like receptor (TLR) ligands and interleukin-1beta (IL-1β) (Sato et al., Nat Immunol, 2005, 6 (11), 1087-95). In response to activation, TAK1 mediates the activation of the nuclear factor κB (NF-κB), c-Jun N-terminal kinase (INK), and p38 pathways (reviewed in Dai et al., Life, 2012, 64(10), 825-834).

Inhibitors of TAK1 have shown potential in models of rheumatoid arthritis (Scarneo et al., Arthritis Research & Therapy, 2019, 21(292)), pancreatic cancer (Melisi et al., J Natl Cancer Inst, 2011, 103:1190-1204) and colitis (Liu et al., Physiol Rep, 2017, 5(7), e13181). Inhibition of TAK1 has also shown potential for the treatment of KRAS (Kirsten rat sarcoma viral oncogene homolog)-dependent cancers (Singh et al., Cell. 2012, 148(4), 639-650).

Various inhibitors of TAK1 have been described in the art, for example a series of TAK1 inhibitors are disclosed by Totzke et al. (Cell Chem Biol. 2017, 24(8), 1029-1039) and in US 2018/0105500 (Derbyshire et al.) and US 2019/0263759 (Derbyshire et al.). However, many of the known TAK1 inhibitors display poor aqueous solubility, poor selectivity for TAK1 and/or poor cellular activity. There is therefore a need in the art for further TAK1 inhibitors with beneficial properties.

### SUMMARY OF THE INVENTION

The present invention provides compounds according to formula (I)

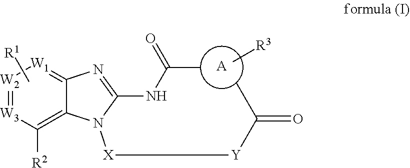

or a tautomer thereof,
wherein,
A is a 6 membered aromatic ring or 5 to 6 membered heteroaromatic ring comprising 1 or 2 atoms selected from the group consisting of N, S and O;
W₁, W₂, W₃ are independently selected from CH and N;
X is linear C₂₋₈alkylene, optionally substituted with one or more groups independently selected from halogen; OH; C₃₋₆cycloalkyl; C₁₋₆alkyl optionally substituted with one or more halogen or OH; and two groups attached to the same carbon atom in the linear C₂₋₈alkylene which together with the atom in the linear C₂₋₈alkylene form a 3- or 4-membered cycloalkyl group; and/or wherein optionally one or more carbon atoms in the alkylene is replaced by a group independently selected from O, NH and S;
Y is NH or O;
R¹ and R² are independently selected from H; C₁₋₆alkyl; C₂₋₆alkenyl; C₂₋₆alkynyl; OC₁₋₆alkyl; OC₂₋₆alkenyl; OC₂₋₆alkynyl; —R^{a}, C(O)R^{a}; C(O)OR^{a}; OCR^{a}; OC(O)(R^{a}); —C₁₋₆ alkylene-R^{a}; —C(O)C₁₋₆alkylene-R^{a}; —C(O)OC₁₋₆alkylene-R^{a}; —OC₁₋₆alkylene-R^{a}; OC(O)C₁₋₆alkylene-R^{a}; and —R^{b}, wherein said alkyl, alkenyl, or alkynyl is optionally substituted with one or more R^{b} groups;
R³ is H, halogen, OH, C₁₋₆alkyl or OC₁₋₆alkyl, wherein said alkyl is optionally substituted with one or more halogen;
   wherein,
R^{a} is selected from C₃₋₈cycloalkyl or 3 to 12 membered heterocycle comprising one, two or three atoms selected from the group consisting of N, S and O; said cycloalkyl or heterocycle optionally being bridged by C₁₋₄alkylene and/or substituted by one or more groups independently selected from C₁₋₄alkyl; 3 to 6 membered heterocycle comprising one, two or three atoms selected from the group consisting of N, S and O; C₃₋₆cycloalkyl; OC₁₋₄alkyl; O-(3 to 6 membered heterocycle comprising one, two or three atoms selected from the group consisting of N, S and O); OC₃₋₆cycloalkyl; C(O)C₁₋₄alkyl; C(O)OC₁₋₄alkyl; OC(O)C₁₋₄alkyl; C(O)NH₂; and —C₁₋₄alkylene-OC₁₋₄alkyl; wherein said alkyl, alkylene, heterocycle or cycloalkyl at R^{a} is optionally substituted by one or more groups independently selected from halogen; OH; O; SH; NH₂; NHC₁₋₄alkyl; or N(C₁₋₄alkyl)₂; and
Each R^{b} is independently selected from C(O)R^{c}; C(O)OR^{c}; OC(O)R^{c}; SR^{c}; SO₂R^{c}; NO₂; CN; NR^{c}R^{d}; N(R^{c})SO₂C₁₋₃alkyl; N(R^{c})C(O)R^{d}; C(O)NR^{c}R^{d}; SO₂NR^{c}R^{d}, halogen; SH; NH₂; and OH; said alkyl optionally being substituted with one or more groups independently selected from halogen, SH, NH₂ and OH and/or wherein optionally one or more carbon atoms in the alkyl is replaced by a group independently selected from O, NH and S; and
R^{c} and R^{d} are independently selected from H; C₁₋₆alkyl; C₂₋₆alkenyl; C₂₋₆alkynyl; —C₁₋₄alkylene-OC₁₋₄alkyl; and C₃₋₈cycloalkyl; said alkyl, alkylene, alkenyl, alkynyl or cycloalkyl optionally being substituted with one or more groups selected from halogen, SH, NH₂, OH and ═O, and/or wherein optionally one or more carbon atoms in the alkyl is replaced by an atom independently selected from O, N and S;
   or a pharmaceutically acceptable salt or solvate thereof.

The present inventors have found that compounds of formula (I) are potent inhibitors of the serine/threonine kinase, TAK1. In particular, the present inventors have demonstrated in an in vitro cell-based p38 phosphorylation assay that compounds of formula (I) display excellent cellular potency. Selectivity is important for any kinase inhibitor. The compounds of the invention display good selectivity for TAK1. For example, compounds of the invention have good selectivity for TAK1 over Interleukin-1 receptor-associated kinase (IRAK).

The present invention further provides a pharmaceutical composition comprising a compound of formula (I) and at least one pharmaceutically acceptable excipient.

The present invention further provides a compound of formula (I), or pharmaceutical composition of the invention, for use as a medicament.

The present invention further provides a method of treating or preventing a disease or disorder comprising administering a pharmaceutically effective amount of a compound of formula (I), or pharmaceutical composition of the present invention, to a subject in need thereof. The present invention also provides a use of a compound of formula (I) in the manufacture of a medicament.

### BRIEF DESCRIPTION OF THE FIGURES

FIG. 1 shows a Western blot obtained in a cell-based p38 phosphorylation assay using a compound of the invention.

### DETAILED DESCRIPTION OF THE INVENTION

The present inventors have found a new class of inhibitors of the serine/threonine kinase, TAK1. As described in the Examples section, various example compounds of the invention have been synthesised and their ability to inhibit TAK1 activity has been assessed using an in vitro TAK1 biochemical assay and an in vitro cell-based p38 phosphorylation assay. The inventors found that compounds of the invention are highly potent inhibitors of TAK1 activity in the vitro biochemical assay. Furthermore, the present inventors have found that compounds of the invention display excellent cellular activity in a p38 phosphorylation assay (see, for example, the experimental results of Biological Examples 1 and 2 below). As such, the compounds of the present invention are of particular interest for use in the treatment or prevention of diseases or disorders that are associated with aberrant TAK1 activity.

Furthermore, the compounds of the present invention display high solubility in aqueous solutions with a solubility of up to about 90 μM being achieved in a solubility assay.

The present invention provides compounds of formula (I):

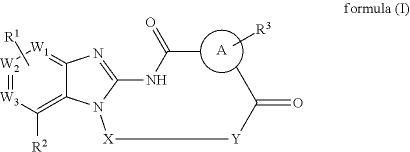

In the compound of formula (I), A is a 6 membered aromatic ring or 5 to 6 membered heteroaromatic ring comprising one or two atoms selected from the group consisting of N, S and O. For example, A may be selected from the group consisting of phenyl, furyl, pyrrolyl, pyrazolyl, pyridyl, pyrimidinyl, pyridazinyl, thienyl, thiazole, isoxazolyl, and isothiazolyl. In the case of a compound in which A is a heteroaromatic ring containing three heteroatoms, the ring may be a triazinyl group (not carrying any R³ substituent). In exemplary embodiments, A is phenyl, pyridyl, thiazole or thienyl. Preferably, A is phenyl or thienyl. More preferably, A is phenyl.

In embodiments wherein A is phenyl, the compound of formula (I) is preferably according to formula (Ia):

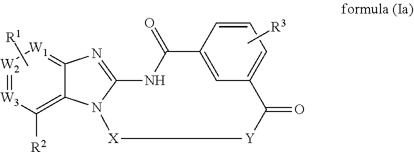

In embodiments wherein A is pyridyl, the compound of formula (I) is preferably according to formula (Ib):

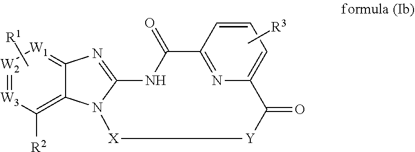

In embodiments wherein A is thiazole, the compound of formula (I) is preferably according to formula (Ic):

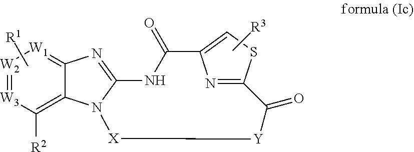

In embodiments wherein A is thienyl, the compound of formula (I) is preferably according to formula (Id):

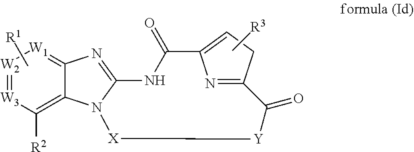

In the compound of formula (I) (and likewise compounds of formula (Ia), (Ib), (Ic), (Id), (Ie) and (If) throughout this section), R³ is selected from the group consisting of H, halogen (for example F, Cl, Br or I), OH, C₁₋₆alkyl or OC₁₋₆alkyl, wherein said alkyl is optionally substituted with one, two or three halogens (for example, F, Cl, Br or I). For example, R³ may be H, F, Cl, Br, I, C₁₋₄alkyl or OC₁₋₄alkyl, wherein said alkyl is optionally substituted with one or two, preferably one, F or Cl. Preferably, R³ is H, F, Cl, methyl, ethyl or methoxy. More preferably, R³ is H or F.

In the compound of formula (I) (and likewise compounds of formula (Ia), (Ib), (Ic), (Id), (Ie) and (If) throughout this section), W₁, W₂, W₃ are independently selected from CH and N. For example, one of W₁, W₂, W₃ may be N and the rest may be CH. In certain exemplary embodiments, W₁, W₂, W₃ are each CH. In other exemplary embodiments, W₁ and W₂ are each CH and W₃ is N. In alternative exemplary embodiments, W₁ and W₃ are each CH and W₂ is N. Preferably, W₁, W₂, W₃ are each CH.

In the compound of formula (I) (and likewise compounds of formula (Ia), (Ib), (Ic), (Id), (Ie) and (If) throughout this section), X is a linear C₂₋₈alkylene, optionally substituted with one or more groups independently selected from halogen (for example, F, Cl, Br, I), OH, C₃₋₆cycloalkyl, and C₁₋₆alkyl optionally substituted with one or more halogen or OH; and two groups attached to the same carbon atom in the linear C₂₋₈alkylene which together with the atom in the linear C₂₋₈alkylene form a 3- or 4-membered cycloalkyl group. For example, X may be a linear C₄₋₈alkylene substituted with one or two groups independently selected from F, Cl, OH and C₁₋₆ alkyl. If the linear C₄₋₈alkylene is substituted by two groups attached to the same carbon atom in the linear C₂₋₈alkylene which together with the atom in the linear C₂₋₈alkylene form a 3- or 4-membered cycloalkyl group, it is preferred for the cycloalkyl group to be cyclopropyl. In certain embodiments, the alkylene of X is substituted with one methyl or one ethyl group. In certain preferred embodiments, the alkylene of X is substituted with one methyl group.

Optionally one or more carbon atoms in the alkylene at the X position is replaced by a group independently selected from O, NH and S (for example an atom independently selected from O and S). For example, one or two carbon atoms in the alkylene at the X position may be replaced by a group independently selected from O, NH and S. In certain exemplary embodiments, one carbon atom in the alkylene is replaced by O or NH, more preferably O.

The present inventors have found that the alkylene at position X, which forms a link between the imidazole moiety and A of the compound of formula (I), provides compounds that display excellent potency for TAK1 inhibition compared to corresponding compounds that do not contain an alkylene linker at position X joining to Y. Without wishing to be bound by theory, the present inventors believe that the surprising potency displayed by compounds of the invention is a result of the rigidity imparted to the compound by the alkylene linker at the X position, which is believed to hold the compound in a particularly effective configuration for binding and inhibiting TAK1. A 4 to 8 carbon alkylene (i.e. C₄₋₈alkylene) at the X position provides the necessary rigidity to the compound. The present inventors have also found that the alkylene linker at position X also surprisingly enhances the compounds aqueous solubility. Optimally, the alkylene at the X position is a 4 carbon to 8 carbon alkylene. Alkylene linkers at the X position that are 5 carbon or 6 carbon in length have been found to be particularly effective. As such, in preferred embodiments, X is a linear C₅alkylene or C₆alkylene. In especially preferred embodiments, X is a linear C₅alkylene.

In certain embodiments, the alkylene of X is substituted by one, two or three groups independently selected from F, Cl, Br, I, OH, C₃₋₆cycloalkyl, and C₁₋₆alkyl optionally substituted with one or more halogen or OH. For example, the alkylene of X may be substituted by one, two or three groups independently selected from F, Cl, OH, methyl or ethyl. In embodiments wherein the alkylene of X is substituted by a C₁₋₆alkyl group, the C₁₋₆alkyl group may substituted by one or more F, Cl or OH. For example, a substituent may be at the first carbon in the X alkylene chain at the benzimidazole (or variant thereof) end of the X group. In such a compound, if there is a single substituent, it gives rise to a chiral centre. In preferred compounds, the substituent is orientated into the page for the compounds as drawn herein. For a substituent that is methyl, the chiral centre is in the (S) orientation. In preferred compounds, the substituent is orientated as shown here within a compound of formula (I):

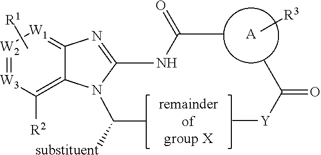

Further, for compounds with X of C₅alkyl chain length, the structure is as follows:

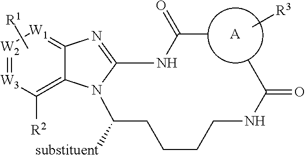

And, for example, in compounds in which X of C₅alkyl chain length, the substituent is methyl and W₁, W₂ and W₃ are all CH, the structure is preferably:

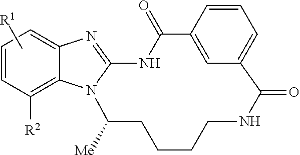

Substitution of the alkylene of X with one or more groups described herein influences the biological activity and/or physicochemical properties of the compounds of formula (I). For example, compounds of formula (I) wherein the alkylene of X is substituted with methyl display enhanced aqueous solubility. Thus, in certain preferred embodiments, the alkylene of X is substituted with one methyl group. For example, X may be —CH(CH₃)(CH₂)₄—. In such a compound, the CH₃ substituent gives rise to a chiral centre. In preferred compounds, the chiral centre is in the (S) orientation.

In certain embodiments, one, two or three carbon atoms in the alkylene of X is/are replaced by a group independently selected from O, NH and S. For example, one or two carbon atoms in the alkylene of X is/are replaced by a group independently selected from O or NH. Replacement of one or more atoms in the alkylene of X with O, NH or S influences the biological activity and/or physicochemical properties of the compounds of formula (I). For example, compounds of formula (I) wherein one carbon atom is replaced by O display notably enhanced aqueous solubility. Thus, in certain preferred embodiments, one carbon atom in the alkylene of X is replaced by O. For example, X may be —(CH₂)₂—O—(CH₂)₂—.

In the compound of formula (I) (and likewise compounds of formula (Ia), (Ib), (Ic), (Id), (Ie) and (If) throughout this section), Y is NH or O. Preferably, Y is NH.

In the compound of formula (I) (and likewise compounds of formula (Ia), (Ib), (Ic), (Id), (Ie) and (If) throughout this section), R¹ and R² are independently selected from H; C₁₋₆alkyl; C₂₋₆alkenyl; C₂₋₆alkynyl; OC₁₋₆alkyl; OC₂₋₆alkenyl; OC₂₋₆alkynyl; —R^{a}, C(O)R^{a}; C(O)OR^{a}; OC(O)(R^{a}); —C₁₋₆alkylene-R^{a}; C(O)C₁₋₆alkylene-R^{a}; —C(O)OC₁₋₆alkylene-R^{a}; —OC₁₋₆alkylene-R^{a}; OC(O)C₁₋₆alkylene-R^{a}; and —R^{b}, wherein said alkyl, alkenyl or alkynyl is optionally substituted with one or more R^{b} groups. In certain embodiments, R¹ and R² are both H. In certain other embodiments, at least of R¹ and R² is not H.

Compounds of formula (I), wherein at least one of R¹ and R² is not H, display enhanced potency for TAK1 inhibition and/or enhanced aqueous solubility.

Thus, in certain preferred embodiments, R¹ is H and R² is selected from C₁₋₆alkyl; C₂₋₆alkenyl; C₂₋₆alkynyl; OC₁₋₆alkyl; OC₂₋₆alkenyl; OC₂₋₆alkynyl; —R^{a}, C(O)R^{a}; C(O)OR^{a}; OC(O)(R^{a}); —C₁₋₆alkylene-R^{a}; and —R^{b}, wherein said alkyl, alkenyl or alkynyl is optionally substituted with one or more R^{b} groups. For example, R² is selected from C₁₋₆alkyl; C₂₋₆alkenyl; C₂₋₆alkynyl; —R^{a}, C(O)R^{a}; C(O)OR^{a}; OC(O)(R^{a}); —C₁₋₆alkylene-R^{a}; and —R^{b}, wherein said alkyl, alkenyl or alkynyl is optionally substituted with one or more R^{b} groups. For example, R² may be C₁₋₄alkyl; C₂₋₄alkenyl; C₂₋₄alkynyl; OC₁₋₄alkyl; OC₂₋₄alkenyl; OC₂₋₄alkynyl; —R^{a}, C(O)R^{a}; C(O)OR^{a}; OC(O)(R^{a}); —C₁₋₄alkylene-R^{a}; and R^{b}, wherein said alkyl, alkenyl or alkynyl is optionally substituted with one, two or three R^{b} groups. For example, R² may be C₁₋₄alkyl; C₂₋₄alkenyl; C₂₋₄alkynyl; R^{a}, C(O)R^{a}; C(O)OR^{a}; OC(O)(R^{a}); —C₁₋₄alkylene-R^{a}; and —R^{b}, wherein said alkyl, alkenyl or alkynyl is optionally substituted with one, two or three R^{b} groups.

Preferably, R¹ is H and R² is —R^{a}, C(O)R^{a}; C(O)OR^{a}; OC(O)(R^{a}); -methylene-R^{a}; or —R^{b}. More preferably, R¹ is H and R² is —R^{a}, C(O)R^{a}; or —R^{b}. In certain exemplary embodiments, R¹ is H and R² is —R^{a}.

In other preferred embodiments, R² is H and R¹ is selected from C₁₋₆alkyl; C₂₋₆alkenyl; C₂₋₆alkynyl; OC₁₋₆alkyl; OC₂₋₆alkenyl; OC₂₋₆alkynyl; —R^{a}, C(O)R^{a}; C(O)OR^{a}; OC(O)(R^{a}); —C₁₋₆alkylene-R^{a}; and —R^{b}, wherein said alkyl, alkenyl or alkynyl is optionally substituted with one or more R^{b} groups. For example, R² is H and R¹ may be C₁₋₄alkyl; C₂₋₄alkenyl; C₂₋₄alkynyl; OC₁₋₄alkyl; OC₂₋₄alkenyl; OC₂₋₄alkynyl; R^{a}, C(O)R^{a}; C(O)OR^{a}; OC(O)(R^{a}); —C₁₋₄alkylene-R^{a}; and —R^{b}, wherein said alkyl, alkenyl or alkynyl is optionally substituted with one, two or three R^{b} groups. Preferably, R² is H and R¹ is methyl; —R^{a}, C(O)R^{a}; C(O)OR^{a}; OC(O)(R^{a}); -methylene-R^{a}; or —R^{b}. More preferably, R² is H and R¹ is methyl; —R^{a}, C(O)R^{a}; or -methylene-R^{a}.

In other preferred embodiments, R¹ and R² are independently selected from C₁₋₆alkyl; C₂₋₆alkenyl; C₂₋₆alkynyl; OC₁₋₆alkyl; OC₂₋₆alkenyl; OC₂₋₆alkynyl; —R^{a}, C(O)R^{a}; C(O)OR^{a}; OC(O)(R^{a}); —C₁₋₆alkylene-R^{a}; and —R^{b}, wherein said alkyl, alkenyl or alkynyl is optionally substituted with one or more R^{b} groups. For example, R¹ and R² may independently be C₁₋₄alkyl; C₂₋₄alkenyl; C₂₋₄alkynyl; OC₁₋₄alkyl; OC₂₋₄alkenyl; OC₂₋₄alkynyl; R^{a}, C(O)R^{a}; C(O)OR^{a}; OC(O)(R^{a}); —C₁₋₄alkylene-R^{a}; and —R^{b}, wherein said alkyl, alkenyl or alkynyl is optionally substituted with one, two or three R^{b} groups. Preferably, R¹ and R² are independently methyl; —R^{a}, C(O)R^{a}; C(O)OR^{a}; OC(O)(R^{a}); -methylene-R^{a}; or —R^{b}. In one exemplary embodiment, R¹ is -methylene-R^{a} and R² is —R^{b}.

In the compound of formula (I), R¹ is at the 5 position or the 6 position of the compound of formula (I). When R¹ is at the 5 position, R¹ is preferably methyl; —R^{a}, C(O)R^{a}; C(O)OR^{a}; OC(O)(R^{a}); -methylene-R^{a}; or —R^{b}, and R² is preferably H or —R^{b}. When R¹ is at the 6 position of the compound of formula (I), R¹ is preferably methyl; —R^{a}, C(O)R^{a}; C(O)OR^{a}; OC(O)(R^{a}); -methylene-R^{a}; or —R^{b}, and R² is preferably H.

In further preferred embodiments, R¹ is at the 5 position and R² is not H. In such an embodiment, R¹ is preferably selected from C₁₋₆alkyl; C₂₋₆alkenyl and C₂₋₆alkynyl, wherein said alkyl, alkenyl, or alkynyl is optionally substituted with one or more R^{b} groups. For example, it may be a branched C₃₋₆alkyl group optionally substituted with one or more R^{b} groups, in particular one or more R^{b} groups selected from SH, NH₂ and OH. For example, R¹ may be a branched C₃₋₆alkyl group substituted with OH, for example it may be —CH(CH₃)OH.

In the compound of formula (I) (and likewise compounds of formula (Ia), (Ib), (Ic), (Id), (Ie) and (If) throughout this section), R^{a} is selected from C₃₋₈cycloalkyl or 3 to 12 membered heterocycle comprising one, two or three atoms selected from the group consisting of N, S and O (preferably N and O); said cycloalkyl or heterocycle optionally being bridged by C₁₋₄alkylene and/or substituted by one or more C₁₋₄alkyl; 3 to 6 membered heterocycle comprising one, two or three atoms selected from the group consisting of N, S and O (preferably N and O); C₃₋₆cycloalkyl; OC₁₋₄alkyl; O-(3 to 6 membered heterocycle comprising one, two or three atoms selected from the group consisting of N, S and O); OC₃₋₆cycloalkyl; C(O)C₁₋₄alkyl; C(O)OC₁₋₄alkyl; OC(O)C₁₋₄alkyl; C(O)NH₂; or —C₁₋₄alkylene-OC₁₋₄alkyl; wherein said alkyl, alkylene, heterocycle or cycloalkyl at R^{a} is optionally substituted by one or more groups independently selected from halogen (preferably F or Cl); OH; O; SH; NH₂; NHC₁₋₄ alkyl; or N(C₁₋₄alkyl)₂.

In certain embodiments, R^{a} is selected from 3 to 8 membered non-aromatic carbocycle; 3 to 8 membered heterocycle or 6 to 12 membered spirocyclic heterocycle comprising one, two or three atoms selected from the group consisting of N, S and O; said non-aromatic carbocycle, heterocycle or spirocyclic heterocycle optionally being bridged by C₁₋₄alkylene and/or substituted by one or more groups independently selected from C₁₋₄alkyl; 3 to 6 membered heterocycle comprising one, two or three atoms selected from the group consisting of N, S and O; C₃₋₆cycloalkyl; OC₁₋₄alkyl; O-(3 to 6 membered heterocycle comprising one, two or three atoms selected from the group consisting of N, S and O); OC₃₋₆cycloalkyl; C(O)C₁₋₄alkyl; C(O)OC₁₋₄alkyl; OC(O)C₁₋₄alkyl; C(O)NH₂; or —C₁₋₄alkylene-OC₁₋₄alkyl, wherein said alkyl, alkylene, heterocycle or cycloalkyl is optionally substituted by one or more groups independently selected from F, Cl; OH; O; SH; NH₂; NHC₁₋₄alkyl; or N(C₁₋₄alkyl)₂.

For the avoidance of doubt, the term “heterocycle” as used herein encompasses any aromatic or non-aromatic cyclic group comprising one or more heteroatoms (i.e. N, O or S). The term “heterocycle” as used herein encompasses bicyclic heterocycle groups such as spirocyclic heterocycles, fused heterocycle and bridged heterocycles, unless otherwise stated. For the avoidance of doubt, a “spirocyclic heterocycle” is a heterocycle which comprise two cyclic structures that are fused at a single atom, wherein at least one of the cyclic structures includes one or more heteroatoms; a “fused heterocycle” is a heterocycle that comprises two cyclic groups with two atoms in common, and wherein at least one of the cyclic structures includes one or more heteroatoms; and a “bridged heterocycle” is a heterocycle that comprises two cyclic groups with three of more atoms in common, wherein two bridgehead atoms are separated by a bridge comprising at least one atom, and wherein at least one of the cyclic structures includes one or more heteroatoms.

For the avoidance of doubt, the term “alkyl” as used herein encompasses linear, branched and cyclic (i.e. cycloalkyl) alkyl groups, unless stated otherwise. The term cycloalkyl as used herein encompasses bicyclic cycloalkyl groups, such as spirocyclic cycloalkyl groups, fused cycloalkyl groups and bridged cycloalkyl groups, unless otherwise stated. For the avoidance of doubt, a “spirocyclic cycloalkyl” is a cycloalkyl which comprise two cyclic structures that are fused at a single carbon atom; a “fused cycloalkyl” is a cycloalkyl that comprises two cyclic groups with two carbon atoms in common; and a “bridged cycloalkyl” is a heterocycle that comprises two cyclic groups with three of more carbon atoms in common and wherein two bridgehead carbon atoms are separated by a bridge comprising at least one carbon atom.

In certain preferred embodiments, R^{a} is 5 to 11 membered heterocycle (for example, a 5 to 8 membered heterocycle or 9 to 11 membered spirocyclic heterocycle) comprising one or two atoms selected from the group consisting of N and O, said heterocycle optionally being bridged by C₁₋₂alkylene and/or substituted by one or two groups independently selected from C₁₋₄alkyl; 3 to 6 membered heterocycle comprising one or two atoms selected from the group consisting of N and O; C₃₋₆cycloalkyl; OC₁₋₄alkyl; C(O)C₁₋₄alkyl; C(O)OC₁₋₄alkyl; OC(O)C₁₋₄alkyl; C(O)NH₂; or —C₁₋₄alkylene-OC₁₋₄alkyl; wherein said alkyl, alkylene, heterocycle or cycloalkyl is optionally substituted by one or more groups independently selected from F, Cl, OH, SH, NH₂; NHC₁₋₄alkyl; or N(C₁₋₄alkyl)₂.

For example, R^{a} is a heterocycle selected from piperazinyl, piperidinyl, morpholinyl, pyrrolidinyl, oxopiperidinyl, homopiperazinyl, pyridinyl, oxodihydropyridinyl, pyrrolidinyl, azetidineyl, triazolyl, oxadiazolenyl, imidazoleyl, oxazolidinoneyl or 9 to 11 membered spirocyclic heterocycle comprising one or two atoms selected from the group consisting of N and O, said heterocycle or spirocyclic heterocycle optionally being bridged by C₁₋₄alkylene and/or substituted with one group independently selected from methyl, ethyl, 3 to 4 membered heterocycle comprising one atoms selected from the group consisting of N, S and O; C₃₋₆cycloalkyl; O-(3 to 6 membered heterocycle comprising one, two or three atoms selected from the group consisting of N, S and O); OC₁₋₄alkyl; C(O)C₁₋₄alkyl; C(O)OC₁₋₄alkyl; OC(O)C₁₋₄alkyl; C(O)NH₂; or —C₁₋₄alkylene-OC₁₋₄alkyl; wherein said alkyl, alkylene, heterocycle, spirocyclic heterocycle (for example non-aromatic spirocyclic heterocycle) or cycloalkyl is optionally substituted by one or more groups independently selected from the group consisting of F, Cl and OH.

Compounds of formula (I) (and likewise compounds of formula (Ia), (Ib), (Ic), (Id), (Ie) and (If) throughout this section) display especially beneficial properties when at least one of R¹ and R² is independently selected from —R^{a}, C(O)R^{a}; C(O)OR^{a}; OC(O)(R^{a}); or -methylene-R^{a}, wherein R^{a} is piperazinyl, piperidinyl, morpholinyl, pyrrolidinyl, oxopiperidinyl, homopiperazinyl, pyridinyl, oxodihydropyridinyl, pyrrolidinyl, azetidineyl, triazole, oxadiazolenyl, imidazoleyl, oxazolidinoneyl, or 9 to 11 membered spirocyclic heterocycle comprising one or two atoms selected from the group consisting of N and O; said heterocycle or spirocyclic heterocycle optionally being bridged by C₁₋₄alkylene and/or substituted with one group independently selected from methyl, ethyl, 3 to 4 membered heterocycle comprising one atoms selected from the group consisting of N, S and O; C₃₋₆cycloalkyl; O-(3 to 6 membered heterocycle comprising one, two or three atoms selected from the group consisting of N, S and O); OC₁₋₄alkyl; C(O)C₁₋₄alkyl; C(O)OC₁₋₄alkyl; OC(O)C₁₋₄alkyl; C(O)NH₂; or —C₁₋₄alkylene-OC₁₋₄alkyl; wherein said alkyl, alkylene, heterocycle, spirocyclic heterocycle (for example non-aromatic spirocyclic heterocycle) or cycloalkyl is optionally substituted by one or more groups independently selected from the group consisting of F, Cl and OH.

For example, it is preferred that one or both of R¹ and R² (for example R¹) is methylene-R^{a} or C(O)R^{a}, where R^{a} is as defined immediately above.

In embodiments wherein the heterocycle at R^{a} is a bridged 5 to 11 membered heterocycle, preferably the heterocycle is bridged by C₁alkylene.

In certain embodiments, R^{a} is selected from piperazinyl, piperidinyl, morpholinyl, pyrrolidinyl, oxopiperidinyl optionally being bridged by C₁₋₄alkylene and/or substituted by one or more C₁₋₄alkyl; 3 to 6 membered heterocycle comprising one, two or three atoms selected from the group consisting of N, S and O; C₃₋₆cycloalkyl; OC₁₋₄alkyl; O-(3 to 6 membered heterocycle comprising one, two or three atoms selected from the group consisting of N, S and O); OC₃₋₆cycloalkyl; C(O)C₁₋₄alkyl; C(O)OC₁₋₄alkyl; OC(O)C₁₋₄alkyl; C(O)NH₂; or —C₁₋₄alkylene-OC₁₋₄alkyl one group independently selected from F; Cl; OH; methyl; 3 to 4 membered heterocycle comprising one atom selected from N or O; OC₁₋₄alkyl; C(O)C₁₋₄alkyl; C(O)OC₁₋₄alkyl; or —C₁₋₂alkylene-OH.

In certain exemplary embodiments, R^{a} is selected from the group consisting of.

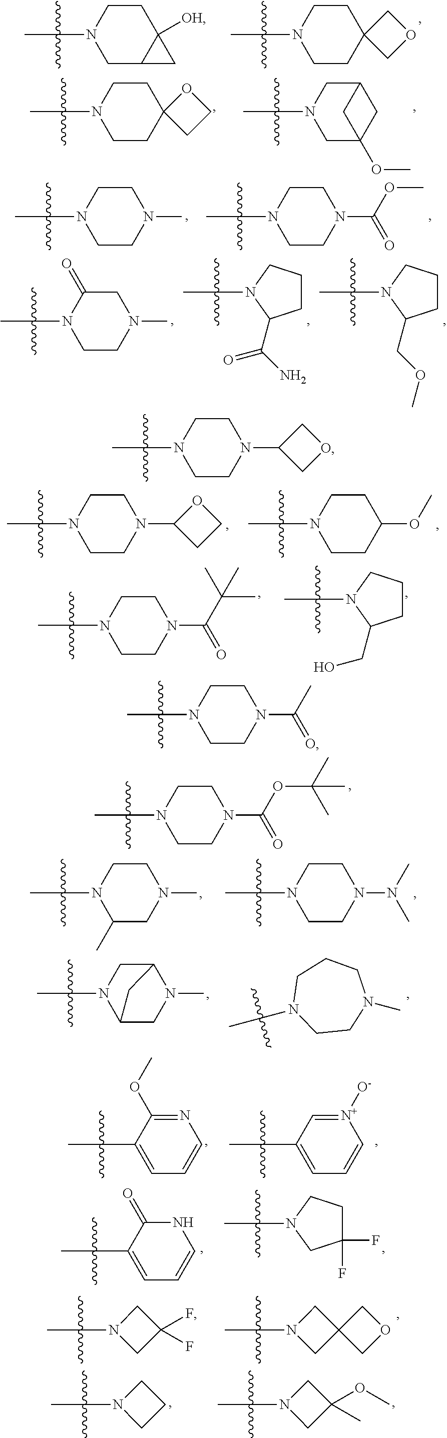

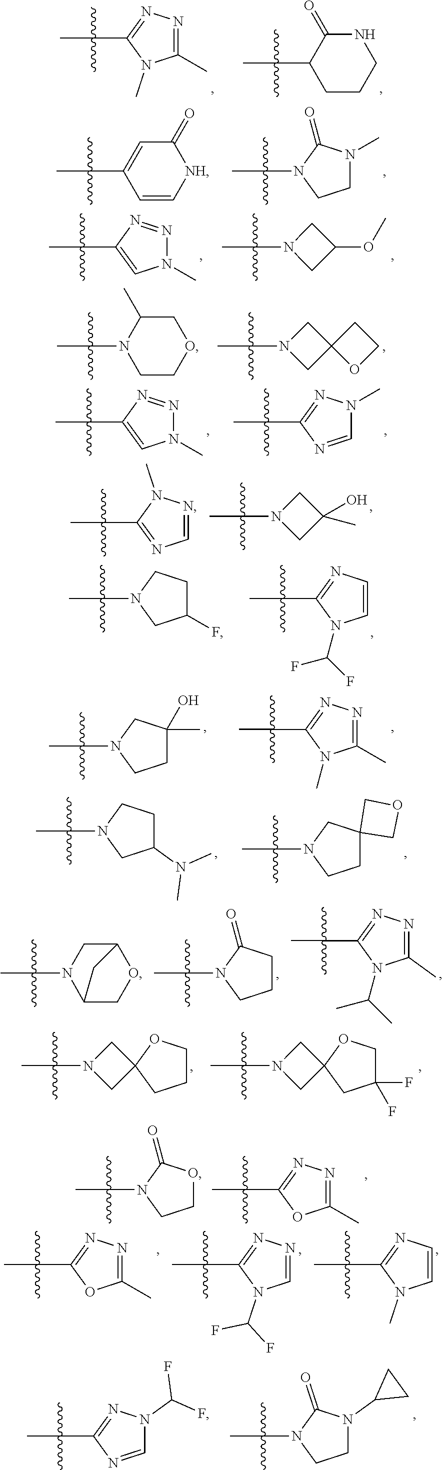

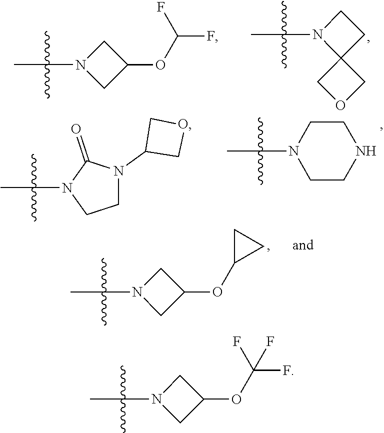

In the compound of formula (I) (and likewise compounds of formula (Ia), (Ib), (Ic), (Id), (Ie) and (If) throughout this section), R^{b} is independently selected from C(O)R^{c}; C(O)OR^{c}; OC(O)R^{c}; SR^{c}; SO₂R^{c}; NO₂; CN; NR^{c}R^{d}; N(R^{c})SO₂C₁₋₃alkyl; N(R^{c})C(O)R^{d}; C(O)NR^{c}R^{d}; SO₂NR^{c}R^{d}; halogen (for example F, Cl, Br, I); SH; NH₂; and OH; said alkyl optionally being substituted with one or more groups independently selected from halogen (for example, F, Cl, Br, I), SH, NH₂ and OH. For example, R^{b} may be independently selected from C(O)R^{c}; C(O)OR^{c}; OC(O)R^{c}; SR^{c}; SO₂R^{c}; NO₂; CN; NR^{c}R^{d}; N(R^{c})SO₂C₁₋₃alkyl; N(R^{c})C(O)R^{d}; C(O)NR^{c}R^{d}; F; Cl; SH; NH₂; or OH; said alkyl optionally being substituted with one or two groups independently selected from F, Cl, SH, NH₂ and OH. For example, R^{b} may be independently selected from C(O)R^{c}; C(O)OR^{c}; OC(O)R^{c}; SR^{c}; SO₂R^{c}; NO₂; CN; NR^{c}R^{d}; N(R^{c})SO₂C₁₋₃alkyl; N(R^{c})C(O)R^{d}; C(O)NR^{c}R^{d}; SH; NH₂; or OH; said alkyl optionally being substituted with one or two groups independently selected from F, Cl, SH, NH₂ and OH. Preferably, R^{b} is C(O)R^{c} or C(O)NR^{c}R^{d}.

In the compound of formula (I) (and likewise compounds of formula (Ia), (Ib), (Ic), (Id), (Ie) and (If) throughout this section), R^{c} and R^{d} are independently selected from H; C₁₋₆alkyl; C₂₋₆alkenyl; C₂₋₆alkynyl; —C₁₋₄alkylene-OC₁₋₄alkyl; and C₃₋₈cycloalkyl; said alkyl, alkenyl, alkynyl or cycloalkyl optionally being substituted with one or more groups selected from halogen, SH, NH₂, OH and ═O, and/or wherein optionally one or more carbon atoms in the alkyl is replaced by an atom independently selected from O, N and S. For example, R^{c} and R^{d} may independently be H; —C₁₋₃alkylene-OC₁₋₃alkyl, C₁₋₃alkyl, C₂₋₃alkenyl; C₂₋₃alkynyl, said alkyl, alkenyl or alkynyl optionally being substituted with one or two groups selected from F, C₁, SH, NH₂ and OH and/or optionally having one or two carbon atoms replaced by an atom independently selected from O, N and S. In exemplary embodiments, R^{c} and R^{d} are independently selected from H, methyl, ethyl, propyl, —C₁₋₂alkylene-OCH₃, —C₁₋₂alkylene(O)OCH₃ (for example, —CH₂(O)OCH₃).

The compound of formula (I) (and likewise compounds of formula (Ia), (Ib), (Ic), (Id), (Ie) and (If) throughout this section) may comprise an isotope atom. As defined herein, an isotope atom is an atom of an element that is not the most common naturally occurring isotope. Deuterium is a safe and stable isotope of hydrogen. In certain embodiments, the compound of formula (I) has a deuterium abundance level greater than the naturally occurring abundance of deuterium. The naturally occurring abundance of deuterium is 0.0156 mol %, wherein mol % is the percentage of the total moles of a sample's hydrogen that is deuterium. Therefore, in 1 mole of naturally occurring hydrogen 0.156 mmol is deuterium, or in a sample of 6.022×10²³ naturally occurring hydrogen atoms there are 9.39×10¹⁹ atoms of deuterium, or in a sample of 6413 naturally occurring hydrogen atoms there is one atom of deuterium. A deuterium abundance level greater than the naturally occurring abundance of deuterium may be at least 1 mol %, 5 mol %, 10 mol %, 50 mol %, 90 mol % or 98 mol % deuterium. In certain embodiments, the compound of formula (I) has a deuterium abundance level of at least 1 mol %, 5 mol %, 10 mol %, 50 mol %, 90 mol % or 98 mol % deuterium. Procedures for preparing deuterated compounds are known in the art. See for example Sajiki, New Horizons of Process Chemistry (2017), Springer, pg 29-40, and Hanson, The Organic Chemistry of Isotopic Labelling (2011), Chapter 3, RSC Publishing. In one exemplary embodiment, R² in the compound of formula (I) is R^{b}, wherein R^{b} is C(O)NR^{c}R^{d}, wherein R^{c} and R^{d} are each —CD₃.

Preferred compounds of the invention are:
wherein the compound is selected from the group consisting of:
1¹H-2,6-diaza-1(2,1)-benzo[d]imidazola-4(1,3)-benzenacyclododecaphane-3,5-dione (Example 1);
1¹H-2,6-diaza-1(2,1)-benzo[d]imidazola-4(1,3)-benzenacycloundecaphane-3,5-dione (Example 2);
1⁵-((4-methylpiperazin-1-yl)methyl)-1¹H-2,6-diaza-1(2,1)-benzo[d]imidazola-4(1,3)-benzenacycloundecaphane-3,5-dione (Example 3);
1⁵-methyl-1¹H-2,6-diaza-1(2,1)-benzo[d]imidazola-4(1,3)-benzenacycloundecaphane-3,5-dione (Example 4);
1¹H-9-oxa-2,6-diaza-1(2,1)-benzo[d]imidazola-4(1,3)-benzenacycloundecaphane-3,5-dione (Example 5);
11-methyl-1¹H-2,6-diaza-1(2,1)-benzo[d]imidazola-4(1,3)-benzenacycloundecaphane-3,5-dione (Example 6);
11-methyl-3,5-dioxo-1¹H-2,6-diaza-1(2,1)-benzo[d]imidazola-4(1,3)-benzenacycloundecaphane-1⁷-carboxylate (Example 7);
11-methyl-1⁷-(4-methylpiperazine-1-carbonyl)-1¹H-2,6-diaza-1(2,1)-benzo[d]imidazola-4(1,3)-benzenacycloundecaphane-3,5-dione (Example 8);
tert-butyl 4-(11-methyl-3,5-dioxo-1¹H-2,6-diaza-1(2,1)-benzo[d]imidazola-4(1,3)-benzenacycloundecaphane-1⁷-carbonyl)piperazine-1-carboxylate (Example 9);
1⁷-(4-methylpiperazine-1-carbonyl)-1¹H-2,6-diaza-1(2,1)-benzo[d]imidazola-4(1,3)-benzenacycloundecaphane-3,5-dione (Example 10);
1⁵-(4-methylpiperazine-1-carbonyl)-1¹H-2,6-diaza-1(2,1)-benzo[d]imidazola-4(1,3)-benzenacycloundecaphane-3,5-dione (Example 11);
1⁵-(4-(oxetan-3-yl)piperazine-1-carbonyl)-1¹H-2,6-diaza-1(2,1)-benzo[d]imidazola-4(1,3)-benzenacycloundecaphane-3,5-dione (Example 12);
tert-butyl 4-(3,5-dioxo-1¹H-2,6-diaza-1(2,1)-benzo[d]imidazola-4(1,3)-benzenacycloundecaphane-1⁵-carbonyl)piperazine-1-carboxylate (Example 13);
tert-butyl 4-(3,5-dioxo-1¹H-2,6-diaza-1(2,1)-benzo[d]imidazola-4(1,3)-benzenacycloundecaphane-1⁷-carbonyl)piperazine-1-carboxylate (Example 14);
11-methyl-1⁷-(piperazine-1-carbonyl)-1¹H-2,6-diaza-1(2,1)-benzo[d]imidazola-4(1,3)-benzenacycloundecaphane-3,5-dione (Example 15);
1⁵-(piperazine-1-carbonyl)-1¹H-2,6-diaza-1(2,1)-benzo[d]imidazola-4(1,3)-benzenacycloundecaphane-3,5-dione (Example 16);
1⁷-(piperazine-1-carbonyl)-1¹H-2,6-diaza-1(2,1)-benzo[d]imidazola-4(1,3)-benzenacycloundecaphane-3,5-dione (Example 17);
(E)-3,5-dioxo-1²,1³-dihydro-1¹H-2,6-diaza-1(2,1)-benzo[d]imidazola-4(1,3)-benzenacycloundecaphane-1⁷-carboxylic acid (Example 18);
(E)-1⁷-(4-(oxetan-3-yl)piperazine-1-carbonyl)-1²,1³-dihydro-1¹H-2,6-diaza-1(2,1)-benzo[d]imidazola-4(1,3)-benzenacycloundecaphane-3,5-dione (Example 19);
(R,E)-1⁷-(2,4-dimethylpiperazine-1-carbonyl)-1²,1³-dihydro-1¹H-2,6-diaza-1(2,1)-benzo[d]imidazola-4(1,3)-benzenacycloundecaphane-3,5-dione (Example 20);
(S,E)-1⁷-(3-(dimethylamino)pyrrolidine-1-carbonyl)-1²,1³-dihydro-1¹H-2,6-diaza-1(2,1)-benzo[d]imidazola-4(1,3)-benzenacycloundecaphane-3,5-dione (Example 21);
(E)-1⁷-((1S,4S)-5-methyl-2,5-diazabicyclo[2.2.1]heptane-2-carbonyl)-1²,1³-dihydro-1¹H-2,6-diaza-1(2,1)-benzo[d]imidazola-4(1,3)-benzenacycloundecaphane-3,5-dione (Example 22);
Example 23: (R,E)-1⁷-(3-(dimethylamino)pyrrolidine-1-carbonyl)-1²,1³-dihydro-1¹H-2,6-diaza-1(2,1)-benzo[d]imidazola-4(1,3)-benzenacycloundecaphane-3,5-dione (Example 23);
(S,E)-1⁷-(2,4-dimethylpiperazine-1-carbonyl)-1²,1³-dihydro-1¹H-2,6-diaza-1(2,1)-benzo[d]imidazola-4(1,3)-benzenacycloundecaphane-3,5-dione (Example 24);
(E)-1⁷-((1R,4R)-5-methyl-2,5-diazabicyclo[2.2.1]heptane-2-carbonyl)-1²,1³-dihydro-1¹H-2,6-diaza-1(2,1)-benzo[d]imidazola-4(1,3)-benzenacycloundecaphane-3,5-dione (Example 25);
(E)-1⁷-(4-methyl-1,4-diazepane-1-carbonyl)-1²,1³-dihydro-1¹H-2,6-diaza-1(2,1)-benzo[d]imidazola-4(1,3)-benzenacycloundecaphane-3,5-dione (Example 26);
(E)-N,N-dimethyl-1⁵-((4-methylpiperazin-1-yl)methyl)-3,5-dioxo-1²,1³-dihydro-1¹H-2,6-diaza-1(2,1)-benzo[d]imidazola-4(1,3)-benzenacycloundecaphane-1⁷-carboxamide (Example 27);
(E)-1⁷-(2-methoxypyridin-3-yl)-1²,1³-dihydro-1¹H-2,6-diaza-1(2,1)-benzo[d]imidazola-4(1,3)-benzenacycloundecaphane-3,5-dione (Example 28);
(R, E)-11-methyl-1⁷-((1R,4R)-5-methyl-2,5-diazabicyclo[2.2.1]heptane-2-carbonyl)-1²,1³-dihydro-1¹H-2,6-diaza-1(2,1)-benzo[d]imidazola-4(1,3)-benzenacycloundecaphane-3,5-dione (Example 29);
(S, E)-11-methyl-1⁷-((1R,4R)-5-methyl-2,5-diazabicyclo[2.2.1]heptane-2-carbonyl)-1²,1³-dihydro-1¹H-2,6-diaza-1(2,1)-benzo[d]imidazola-4(1,3)-benzenacycloundecaphane-3,5-dione (Example 30);
(E)-N,N,11-trimethyl-3,5-dioxo-1²,1³-dihydro-1¹H-2,6-diaza-1(2,1)-benzo[d]imidazola-4(1,3)-benzenacycloundecaphane-1⁷-carboxamide (Example 31 and 32);
((E)-3-(11-methyl-3,5-dioxo-1²,1³-dihydro-1¹H-2,6-diaza-1(2,1)-benzo[d]imidazola-4(1,3)-benzenacycloundecaphane-1⁷-yl)pyridine 1-oxide (Example 33);
(E)-N,11-dimethyl-3,5-dioxo-1²,1³-dihydro-1¹H-2,6-diaza-1(2,1)-benzo[d]imidazola-4(1,3)-benzenacycloundecaphane-1⁷-carboxamide (Example 34);
(E)-11-methyl-1⁷-(2-oxo-1,2-dihydropyridin-3-yl)-1²,1³-dihydro-1¹H-2,6-diaza-1(2,1)-benzo[d]imidazola-4(1,3)-benzenacycloundecaphane-3,5-dione (Example 35 and 36);
(E)-1⁷-(3,3-difluoropyrrolidine-1-carbonyl)-11-methyl-1²,1³-dihydro-1¹H-2,6-diaza-1(2,1)-benzo[d]imidazola-4(1,3)-benzenacycloundecaphane-3,5-dione (Example 37);
methyl (E)-N-methyl-N-(11-methyl-3,5-dioxo-1²,1³-dihydro-1¹H-2,6-diaza-1(2,1)-benzo[d]imidazola-4(1,3)-benzenacycloundecaphane-1⁷-carbonyl)glycinate (Example 38);
(E)-N-cyclobutyl-N,11-dimethyl-3,5-dioxo-1²,1³-dihydro-1¹H-2,6-diaza-1(2,1)-benzo[d]imidazola-4(1,3)-benzenacycloundecaphane-1⁷-carboxamide (Example 39);
(E)-1⁷-(3,3-difluoroazetidine-1-carbonyl)-11-methyl-1²,1³-dihydro-1¹H-2,6-diaza-1(2,1)-benzo[d]imidazola-4(1,3)-benzenacycloundecaphane-3,5-dione (Example 40);
(E)-N-(3-hydroxycyclobutyl)-N,11-dimethyl-3,5-dioxo-1²,1³-dihydro-1¹H-2,6-diaza-1(2,1)-benzo[d]imidazola-4(1,3)-benzenacycloundecaphane-1⁷-carboxamide (Example 41);
(E)-11-methyl-1⁷-(2-oxa-6-azaspiro[3.3]heptane-6-carbonyl)-1²,1³-dihydro-1¹H-2,6-diaza-1(2,1)-benzo[d]imidazola-4(1,3)-benzenacycloundecaphane-3,5-dione (Example 42);
(E)-1⁷-(azetidine-1-carbonyl)-11-methyl-1²,1³-dihydro-1¹H-2,6-diaza-1(2,1)-benzo[d]imidazola-4(1,3)-benzenacycloundecaphane-3,5-dione (Example 43);
(S,E)-N,N,7-trimethyl-3,5-dioxo-1²,1³-dihydro-1¹H-2,6-diaza-1(2,1)-benzo[d]imidazola-4(1,3)-benzenacycloundecaphane-1⁷-carboxamide (Example 44);
(E)-N-(11-methyl-3,5-dioxo-1²,1³-dihydro-1¹H-2,6-diaza-1(2,1)-benzo[d]imidazola-4(1,3)-benzenacycloundecaphane-1⁷-yl)acetamide (Example 45);
(R,E)-N,N,11-trimethyl-3,5-dioxo-1²,1³-dihydro-1¹H-2,6-diaza-1(2,1)-benzo[d]imidazola-4(1,3)-benzenacycloundecaphane-1⁷-sulfonamide (Example 46);
(E)-N-isopropyl-N,11-dimethyl-3,5-dioxo-1²,1³-dihydro-1¹H-2,6-diaza-1(2,1)-benzo[d]imidazola-4(1,3)-benzenacycloundecaphane-1⁷-carboxamide (Example 47 and 48);
(E)-1⁷-(3-methoxy-3-methylazetidine-1-carbonyl)-11-methyl-1²,1³-dihydro-1¹H-2,6-diaza-1(2,1)-benzo[d]imidazola-4(1,3)-benzenacycloundecaphane-3,5-dione (Example 49 and 50);
(E)-N,N,10-trimethyl-3,5-dioxo-1²,1³-dihydro-1¹H-2,6-diaza-1(2,1)-benzo[d]imidazola-4(1,3)-benzenacycloundecaphane-1⁷-carboxamide (Example 51 and 52);
(S,E)-1⁶-chloro-N,N,11-trimethyl-3,5-dioxo-1²,1³-dihydro-1¹H-2,6-diaza-1(2,1)-benzo[d]imidazola-4(1,3)-benzenacycloundecaphane-1⁷-carboxamide (Example 53 and 54);
(R,E)-1⁶-chloro-N,N,11-trimethyl-3,5-dioxo-1²,1³-dihydro-1¹H-2,6-diaza-1(2,1)-benzo[d]imidazola-4(1,3)-benzenacycloundecaphane-1⁷-carboxamide (Example 55 and 56);
(R,E)-1⁷-(4,5-dimethyl-4H-1,2,4-triazol-3-yl)-11-methyl-1²,1³-dihydro-1¹H-2,6-diaza-1(2,1)-benzo[d]imidazola-4(1,3)-benzenacycloundecaphane-3,5-dione (Example 57);
(E)-11-methyl-1⁷-(2-oxopiperidin-3-yl)-1²,1³-dihydro-1¹H-2,6-diaza-1(2,1)-benzo[d]imidazola-4(1,3)-benzenacycloundecaphane-3,5-dione (Examples 58 to 61);
(E)-11-methyl-1⁷-(2-oxo-1,2-dihydropyridin-4-yl)-1²,1³-dihydro-1¹H-2,6-diaza-1(2,1)-benzo[d]imidazola-4(1,3)-benzenacycloundecaphane-3,5-dione (Example 62 and 63);
(R,E)-11-methyl-N,N-bis(methyl-d₃)-3,5-dioxo-1²,1³-dihydro-1¹H-2,6-diaza-1(2,1)-benzo[d]imidazola-4(1,3)-benzenacycloundecaphane-1⁷-carboxamide (Example 64);
(R,E)-11-methyl-1⁷-(3-methyl-2-oxoimidazolidin-1-yl)-1²,1³-dihydro-1¹H-2,6-diaza-1(2,1)-benzo[d]imidazola-4(1,3)-benzenacycloundecaphane-3,5-dione (Example 65);
(R,E)-11-methyl-1⁷-(1-methyl-1H-1,2,3-triazol-4-yl)-1²,1³-dihydro-1¹H-2,6-diaza-1(2,1)-benzo[d]imidazola-4(1,3)-benzenacycloundecaphane-3,5-dione (Example 66);
(E)-11-ethyl-N,N-dimethyl-3,5-dioxo-1²,1³-dihydro-1¹H-2,6-diaza-1(2,1)-benzo[d]imidazola-4(1,3)-benzenacycloundecaphane-1⁷-carboxamide (Example 67 and 68);
(R,E)-1⁵-(2-hydroxypropan-2-yl)-N,N,11-trimethyl-3,5-dioxo-1²,1³-dihydro-1¹H-2,6-diaza-1(2,1)-benzo[d]imidazola-4(1,3)-benzenacycloundecaphane-1⁷-carboxamide (Example 69);
((E)-N-(2-fluoroethyl)-N,11-dimethyl-3,5-dioxo-1²,1³-dihydro-1¹H-2,6-diaza-1(2,1)-benzo[d]imidazola-4(1,3)-benzenacycloundecaphane-1⁷-carboxamide (Example 70 and 71);
(E)-N-(2,2-difluoroethyl)-N,11-dimethyl-3,5-dioxo-1²,1³-dihydro-1¹H-2,6-diaza-1(2,1)-benzo[d]imidazola-4(1,3)-benzenacycloundecaphane-1⁷-carboxamide (Example 72 and 73);
(S,E)-11-methyl-1⁷-(3-methyl-2-oxoimidazolidin-1-yl)-1²,1³-dihydro-1¹H-2,6-diaza-1(2,1)-benzo[d]imidazola-4(1,3)-benzenacycloundecaphane-3,5-dione (Example 74);
(E)-11-methyl-1⁷-((R)-3-methylmorpholine-4-carbonyl)-1²,1³-dihydro-1¹H-2,6-diaza-1(2,1)-benzo[d]imidazola-4(1,3)-benzenacycloundecaphane-3,5-dione (Example 75 and 76);
(E)-11-methyl-1⁷-((S)-3-methylmorpholine-4-carbonyl)-1²,1³-dihydro-1¹H-2,6-diaza-1(2,1)-benzo[d]imidazola-4(1,3)-benzenacycloundecaphane-3,5-dione (Example 77 and 78);
(E)-11-methyl-1⁷-(1-oxa-6-azaspiro[3.3]heptane-6-carbonyl)-1²,1³-dihydro-1¹H-2,6-diaza-1(2,1)-benzo[d]imidazola-4(1,3)-benzenacycloundecaphane-3,5-dione (Example 79 and 80);
(E)-1⁷-(3-methoxyazetidine-1-carbonyl)-11-methyl-1²,1³-dihydro-1¹H-2,6-diaza-1(2,1)-benzo[d]imidazola-4(1,3)-benzenacycloundecaphane-3,5-dione (Example 81 and 82);
(R,E)-11-methyl-1⁷-(1-methyl-1H-1,2,4-triazol-3-yl)-1²,1³-dihydro-1¹H-2,6-diaza-1(2,1)-benzo[d]imidazola-4(1,3)-benzenacycloundecaphane-3,5-dione (Example 83 and 84)
(R,E)-11-methyl-1⁷-(1-methyl-1H-1,2,4-triazol-5-yl)-1²,1³-dihydro-1¹H-2,6-diaza-1(2,1)-benzo[d]imidazola-4(1,3)-benzenacycloundecaphane-3,5-dione (Example 85 and 86);
(S,E)-11-methyl-1⁷-(1-methyl-1H-1,2,3-triazol-4-yl)-1²,1³-dihydro-1¹H-2,6-diaza-1(2,1)-benzo[d]imidazola-4(1,3)-benzenacycloundecaphane-3,5-dione (Example 87);
(E)-1⁷-((S)-3-fluoropyrrolidine-1-carbonyl)-11-methyl-1²,1³-dihydro-1¹H-2,6-diaza-1(2,1)-benzo[d]imidazola-4(1,3)-benzenacycloundecaphane-3,5-dione (Example 88 and 89);
(E)-11-methyl-1⁷-(1-methyl-1H-imidazol-2-yl)-1²,1³-dihydro-1¹H-2,6-diaza-1(2,1)-benzo[d]imidazola-4(1,3)-benzenacycloundecaphane-3,5-dione (Example 90 and 91);
(E)-1⁷-((R)-3-fluoropyrrolidine-1-carbonyl)-11-methyl-1²,1³-dihydro-1¹H-2,6-diaza-1(2,1)-benzo[d]imidazola-4(1,3)-benzenacycloundecaphane-3,5-dione (Example 92 and 93);
(R,E)-1⁷-(3-hydroxy-3-methylazetidine-1-carbonyl)-11-methyl-1²,1³-dihydro-1¹H-2,6-diaza-1(2,1)-benzo[d]imidazola-4(1,3)-benzenacycloundecaphane-3,5-dione (Example 94);
(S,E)-1⁷-(3-hydroxy-3-methylazetidine-1-carbonyl)-11-methyl-1²,1³-dihydro-1¹H-2,6-diaza-1(2,1)-benzo[d]imidazola-4(1,3)-benzenacycloundecaphane-3,5-dione (Example 95);
(S,E)-1⁷-(4,5-dimethyl-4H-1,2,4-triazol-3-yl)-11-methyl-1²,1³-dihydro-1¹H-2,6-diaza-1(2,1)-benzo[d]imidazola-4(1,3)-benzenacycloundecaphane-3,5-dione (Example 96);
(E)-1⁶-(4-methylpiperazine-1-carbonyl)-1²,1³-dihydro-1¹H-2,6-diaza-1(2,1)-benzo[d]imidazola-4(1,3)-benzenacycloundecaphane-3,5-dione (Example 97);
(E)-1⁶-(4-(oxetan-3-yl)piperazine-1-carbonyl)-1²,1³-dihydro-1¹H-2,6-diaza-1(2,1)-benzo[d]imidazola-4(1,3)-benzenacycloundecaphane-3,5-dione (Example 98);
(E)-1⁵-fluoro-1²,1³-dihydro-1¹H-2,6-diaza-1(2,1)-benzo[d]imidazola-4(1,3)-benzenacycloundecaphane-3,5-dione (Example 99);
(E)-1⁶-(piperazine-1-carbonyl)-1²,1³-dihydro-1 ¹H-2,6-diaza-1(2,1)-benzo[d]imidazola-4(1,3)-benzenacycloundecaphane-3,5-dione (Example 100);
(E)-1⁶-((4-methylpiperazin-1-yl)methyl)-1²,1³-dihydro-1¹H-2,6-diaza-1(2,1)-benzo[d]imidazola-4(1,3)-benzenacycloundecaphane-3,5-dione (Example 101);
(E)-1⁵-((4-methylpiperazin-1-yl)methyl)-1²,1³-dihydro-1¹H-2,6-diaza-1(2,1)-benzo[d]imidazola-4(2,6)-pyridinacycloundecaphane-3,5-dione (Example 102);
(E)-1⁵-((4-methylpiperazin-1-yl)methyl)-1²,1³-dihydro-1¹H-2,6-diaza-1(2,1)-benzo[d]imidazola-4(2,6)-pyridinacycloundecaphane-3,5-dione (Example 103);
(1²E,4²Z)-1⁵-((4-methylpiperazin-1-yl)methyl)-1²,1³-dihydro-1¹H-2,6-diaza-1(2,1)-benzo[d]imidazola-4(4,2)-thiophenacycloundecaphane-3,5-dione (Example 104);
(E)-4⁶-fluoro-1⁵-((4-methylpiperazin-1-yl)methyl)-1²,1³-dihydro-1¹H-2,6-diaza-1(2,1)-benzo[d]imidazola-4(1,3)-benzenacycloundecaphane-3,5-dione (Example 105);
(S,E)-1⁷-((R)-3-hydroxy-3-methylpyrrolidine-1-carbonyl)-11-methyl-1²,1³-dihydro-1¹H-2,6-diaza-1(2,1)-benzo[d]imidazola-4(1,3)-benzenacycloundecaphane-3,5-dione (Example 106);
(S,E)-1⁷-(3-cyclopropoxyazetidine-1-carbonyl)-11-methyl-1²,1³-dihydro-1¹H-2,6-diaza-1(2,1)-benzo[d]imidazola-4(1,3)-benzenacycloundecaphane-3,5-dione (Example 107);
(1²Z,4²E)-N,N,5-trimethyl-2,11-dioxo-4²,4³-dihydro-4¹H-3,10-diaza-1(4,2)-thiazola-4(2,1)-benzo[d]imidazolacycloundecaphane-4⁷-carboxamide (Example 108 and 109);
(S,E)-11-methyl-1⁷-(3-(trifluoromethoxy)azetidine-1-carbonyl)-1²,1³-dihydro-11H-2,6-diaza-1(2,1)-benzo[d]imidazola-4(1,3)-benzenacycloundecaphane-3,5-dione (Example 110);
(S,E)-1⁷-((1S,4S)-2-oxa-5-azabicyclo[2.2.1]heptane-5-carbonyl)-11-methyl-1²,1³-dihydro-1¹H-2,6-diaza-1(2,1)-benzo[d]imidazola-4(1,3)-benzenacycloundecaphane-3,5-dione (Example 111);
(S,E)-1⁷-((1R,4R)-2-oxa-5-azabicyclo[2.2.1]heptane-5-carbonyl)-11-methyl-1²,1³-dihydro-1H-2,6-diaza-1(2,1)-benzo[d]imidazola-4(1,3)-benzenacycloundecaphane-3,5-dione (Example 112);
(S,E)-11-methyl-1⁷-(2-oxopyrrolidin-1-yl)-1²,1³-dihydro-1¹H-2,6-diaza-1(2,1)-benzo[d]imidazola-4(1,3)-benzenacycloundecaphane-3,5-dione (Example 113);
(S,E)-11-methyl-1⁷-(2-oxa-6-azaspiro[3.3]heptane-6-carbonyl)-1²,1³-dihydro-1¹H-2,6-diaza-1(2,1)-benzo[d]imidazola-4(1,3)-benzenacycloundecaphane-3,5-dione (Example 114);
(S,E)-11-methyl-1⁷-(2-oxa-6-azaspiro[3.4]octane-6-carbonyl)-1²,1³-dihydro-1¹H-2,6-diaza-1(2,1)-benzo[d]imidazola-4(1,3)-benzenacycloundecaphane-3,5-dione (Example 115);
(S,E)-11-methyl-1⁷-(5-oxa-2-azaspiro[3.4]octane-2-carbonyl)-1²,1³-dihydro-1¹H-2,6-diaza-1(2,1)-benzo[d]imidazola-4(1,3)-benzenacycloundecaphane-3,5-dione (Example 116);
(S,E)-1⁷-(7,7-difluoro-5-oxa-2-azaspiro[3.4]octane-2-carbonyl)-11-methyl-1²,1³-dihydro-1¹H-2,6-diaza-1(2,1)-benzo[d]imidazola-4(1,3)-benzenacycloundecaphane-3,5-dione (Example 117);
(S,E)-11-methyl-N,N-bis(methyl-d₃)-3,5-dioxo-1²,1³-dihydro-1¹H-2,6-diaza-1(2,1)-benzo[d]imidazola-4(1,3)-benzenacycloundecaphane-1⁷-carboxamide (Example 118);
(S,E)-11-methyl-1⁴-(2-oxopyrrolidin-1-yl)-1²,1³-dihydro-1¹H-2,6-diaza-1(2,3)-imidazo[4,5-c]pyridina-4(1,3)-benzenacycloundecaphane-3,5-dione (Example 119);
(11S,E)-11-methyl-1⁷-(1-oxa-6-azaspiro[3.4]octane-6-carbonyl)-1²,1³-dihydro-1¹H-2,6-diaza-1(2,1)-benzo[d]imidazola-4(1,3)-benzenacycloundecaphane-3,5-dione (Examples 120 and 121);
(S,E)-1⁴-(3-cyclopropyl-2-oxoimidazolidin-1-yl)-11-methyl-1²,1³-dihydro-1¹H-2,6-diaza-1(2,3)-imidazo[4,5-c]pyridina-4(1,3)-benzenacycloundecaphane-3,5-dione (Example 122);
(S,E)-1⁵-(2-hydroxypropan-2-yl)-N,N,11-trimethyl-3,5-dioxo-1²,1³-dihydro-1¹H-2,6-diaza-1(2,1)-benzo[d]imidazola-4(1,3)-benzenacycloundecaphane-1⁷-carboxamide (Example 123);
(S,E)-N,N,11-trimethyl-1⁵-((4-methylpiperazin-1-yl)methyl)-3,5-dioxo-1²,1³-dihydro-1¹H-2,6-diaza-1(2,1)-benzo[d]imidazola-4(1,3)-benzenacycloundecaphane-1⁷-carboxamide (Example 124);
(S,E)-N,N,11-trimethyl-1⁵-(morpholinomethyl)-3,5-dioxo-1²,1³-dihydro-1¹H-2,6-diaza-1(2,1)-benzo[d]imidazola-4(1,3)-benzenacycloundecaphane-1⁷-carboxamide (Example 125);
(11S,E)-1⁴-(3-fluoro-2-oxopyrrolidin-1-yl)-11-methyl-1²,1³-dihydro-1¹H-2,6-diaza-1(2,3)-imidazo[4,5-c]pyridina-4(1,3)-benzenacycloundecaphane-3,5-dione (Examples 126 and 127);
(S,E)-N,N,11-trimethyl-3,5-dioxo-1²,1³-dihydro-1¹H-2,6-diaza-1(2,3)-imidazo[4,5-c]pyridina-4(1,3)-benzenacycloundecaphane-1⁴-carboxamide (Example 128);
(S,E)-11-methyl-1⁷-(1-(oxetan-3-yl)-1H-1,2,3-triazol-4-yl)-1²,1³-dihydro-1¹H-2,6-diaza-1(2,1)-benzo[d]imidazola-4(1,3)-benzenacycloundecaphane-3,5-dione (Example 129);
(S,E)-11-methyl-1⁴-(3-methyl-2-oxoimidazolidin-1-yl)-1²,1³-dihydro-1¹H-2,6-diaza-1(2,3)-imidazo[4,5-c]pyridina-4(1,3)-benzenacycloundecaphane-3,5-dione (Example 130);
(E)-N,N-dimethyl-3′,5′-dioxospiro[cyclopropane-1,11′-2,6-diaza-1(2,1)-benzo[d]imidazola-4(1,3)-benzenacycloundecaphane]-7′-carboxamide (Example 131);
(E)-11-methyl-1⁷-(5-methyl-1,3,4-oxadiazol-2-yl)-1²,1³-dihydro-1¹H-2,6-diaza-1(2,1)-benzo[d]imidazola-4(1,3)-benzenacycloundecaphane-3,5-dione (Examples 132 and 133);
(S,E)-1⁷-(4-isopropyl-5-methyl-4H-1,2,4-triazol-3-yl)-11-methyl-1²,1³-dihydro-1¹H-2,6-diaza-1(2,1)-benzo[d]imidazola-4(1,3)-benzenacycloundecaphane-3,5-dione (Example 134);
(E)-11-methyl-1⁷-(1-methyl-1H-imidazol-2-yl)-1²,1³-dihydro-1¹H-2,6-diaza-1(2,1)-benzo[d]imidazola-4(1,3)-benzenacycloundecaphane-3,5-dione (Examples 135 and
136);
(E)-1⁷-(1-(difluoromethyl)-1H-1,2,4-triazol-3-yl)-11-methyl-1²,1³-dihydro-1¹H-2,6-diaza-1(2,1)-benzo[d]imidazola-4(1,3)-benzenacycloundecaphane-3,5-dione (Examples 137 and 138);
(E)-1⁷-(1-(difluoromethyl)-1H-1,2,4-triazol-5-yl)-11-methyl-1²,1³-dihydro-1¹H-2,6-diaza-1(2,1)-benzo[d]imidazola-4(1,3)-benzenacycloundecaphane-3,5-dione (Examples 139 and 140);
(E)-1⁷-(3-cyclopropyl-2-oxoimidazolidin-1-yl)-11-methyl-1²,1³-dihydro-1¹H-2,6-diaza-1(2,1)-benzo[d]imidazola-4(1,3)-benzenacycloundecaphane-3,5-dione (Examples 141 and 142);
(E)-11-methyl-1⁷-(3-(oxetan-3-yl)-2-oxoimidazolidin-1-yl)-1²,1³-dihydro-1¹H-2,6-diaza-1(2,1)-benzo[d]imidazola-4(1,3)-benzenacycloundecaphane-3,5-dione (Examples 143 and 144);
((S,E)-N,N,11-trimethyl-3,5-dioxo-1²,1³-dihydro-1¹H-2,6-diaza-1(2,1)-imidazo[4,5-c]pyridina-4(1,3)-benzenacycloundecaphane-1⁷-carboxamide (Example 145);
(11S,E)-1⁷-(3-fluoro-2-oxopyrrolidin-1-yl)-11-methyl-1²,1³-dihydro-1¹H-2,6-diaza-1(2,1)-benzo[d]imidazola-4(1,3)-benzenacycloundecaphane-3,5-dione (Example 146); and
(S,E)-11-methyl-1⁷-(3-methyl-1,2,4-oxadiazol-5-yl)-1²,1³-dihydro-1¹H-2,6-diaza-1(2,1)-benzo[d]imidazola-4(1,3)-benzenacycloundecaphane-3,5-dione (Example 147)
   or pharmaceutically acceptable salts or solvates thereof.

The following compounds are further particular compounds of the invention:
(S,E)-1⁵-(2-hydroxypropan-2-yl)-11-methyl-1⁷-(3-methyl-2-oxoimidazolidin-1-yl)-1²,1³-dihydro-1¹H-2,6-diaza-1(2,1)-benzo[d]imidazola-4(1,3)-benzenacycloundecaphane-3,5-dione;
(S,E)-1⁵-(2-hydroxypropan-2-yl)-11-methyl-1⁷-(2-oxopyrrolidin-1-yl)-1²,1³-dihydro-1¹H-2,6-diaza-1(2,1)-benzo[d]imidazola-4(1,3)-benzenacycloundecaphane-3,5-dione;
(S,E)-1⁵-(2-hydroxypropan-2-yl)-11-methyl-1⁷-(1-methyl-1H-1,2,3-triazol-4-yl)-1²,1³-dihydro-1H-2,6-diaza-1(2,1)-benzo[d]imidazola-4(1,3)-benzenacycloundecaphane-3,5-dione;
(S,E)-1⁵-(2-hydroxypropan-2-yl)-11-methyl-1⁷-(1-methyl-1H-1,2,4-triazol-5-yl)-1²,1³-dihydro-1H-2,6-diaza-1(2,1)-benzo[d]imidazola-4(1,3)-benzenacycloundecaphane-3,5-dione;
(S,E)-1⁵-(2-hydroxypropan-2-yl)-11-methyl-1⁷-(2-oxooxazolidin-3-yl)-1²,1³-dihydro-1¹H-2,6-diaza-1(2,1)-benzo[d]imidazola-4(1,3)-benzenacycloundecaphane-3,5-dione; and
(S,E)-1⁷-((S)-3-fluoropyrrolidine-1-carbonyl)-1⁵-(2-hydroxypropan-2-yl)-11-methyl-1²,1³-dihydro-1¹H-2,6-diaza-1(2,1)-benzo[d]imidazola-4(1,3)-benzenacycloundecaphane-3,5-dione.

Preferred compounds include:
1¹H-2,6-diaza-1(2,1)-benzo[d]imidazola-4(1,3)-benzenacycloundecaphane-3,5-dione (Example 2);
1⁵-((4-methylpiperazin-1-yl)methyl)-1¹H-2,6-diaza-1(2,1)-benzo[d]imidazola-4(1,3)-benzenacycloundecaphane-3,5-dione (Example 3);
1⁵-methyl-1¹H-2,6-diaza-1(2,1)-benzo[d]imidazola-4(1,3)-benzenacycloundecaphane-3,5-dione (Example 4);
1¹-methyl-1¹H-2,6-diaza-1(2,1)-benzo[d]imidazola-4(1,3)-benzenacycloundecaphane-3,5-dione (Example 6);
1¹-methyl-3,5-dioxo-1 ¹H-2,6-diaza-1(2,1)-benzo[d]imidazola-4(1,3)-benzenacycloundecaphane-1⁷-carboxylate (Example 7);
1¹-methyl-1⁷-(4-methylpiperazine-1-carbonyl)-1¹H-2,6-diaza-1(2,1)-benzo[d]imidazola-4(1,3)-benzenacycloundecaphane-3,5-dione (Example 8);
tert-butyl 4-(11-methyl-3,5-dioxo-1H-2,6-diaza-1(2,1)-benzo[d]imidazola-4(1,3)-benzenacycloundecaphane-1⁷-carbonyl)piperazine-1-carboxylate (Example 9);
1⁷-(4-methylpiperazine-1-carbonyl)-1H-2,6-diaza-1(2,1)-benzo[d]imidazola-4(1,3)-benzenacycloundecaphane-3,5-dione (Example 10);
1⁵-(4-methylpiperazine-1-carbonyl)-1¹H-2,6-diaza-1(2,1)-benzo[d]imidazola-4(1,3)-benzenacycloundecaphane-3,5-dione (Example 11);
1⁵-(4-(oxetan-3-yl)piperazine-1-carbonyl)-1¹H-2,6-diaza-1(2,1)-benzo[d]imidazola-4(1,3)-benzenacycloundecaphane-3,5-dione (Example 12);
tert-butyl 4-(3,5-dioxo-1¹H-2,6-diaza-1(2,1)-benzo[d]imidazola-4(1,3)-benzenacycloundecaphane-1⁵-carbonyl)piperazine-1-carboxylate (Example 13);
1¹-methyl-1⁷-(piperazine-1-carbonyl)-1¹H-2,6-diaza-1(2,1)-benzo[d]imidazola-4(1,3)-benzenacycloundecaphane-3,5-dione (Example 15);
1⁵-(piperazine-1-carbonyl)-1¹H-2,6-diaza-1(2,1)-benzo[d]imidazola-4(1,3)-benzenacycloundecaphane-3,5-dione (Example 16);
1⁷-(piperazine-1-carbonyl)-1¹H-2,6-diaza-1(2,1)-benzo[d]imidazola-4(1,3)-benzenacycloundecaphane-3,5-dione (Example 17);
(E)-3,5-dioxo-1²,1³-dihydro-1¹H-2,6-diaza-1(2,1)-benzo[d]imidazola-4(1,3)-benzenacycloundecaphane-1⁷-carboxylic acid (Example 18);
(E)-1⁷-(4-(oxetan-3-yl)piperazine-1-carbonyl)-1²,1³-dihydro-1¹H-2,6-diaza-1(2,1)-benzo[d]imidazola-4(1,3)-benzenacycloundecaphane-3,5-dione (Example 19);
(S,E)-1⁷-(3-(dimethylamino)pyrrolidine-1-carbonyl)-1²,1³-dihydro-1¹H-2,6-diaza-1(2,1)-benzo[d]imidazola-4(1,3)-benzenacycloundecaphane-3,5-dione (Example 21);
(E)-1⁷-((1S,4S)-5-methyl-2,5-diazabicyclo[2.2.1]heptane-2-carbonyl)-1²,1³-dihydro-1¹H-2,6-diaza-1(2,1)-benzo[d]imidazola-4(1,3)-benzenacycloundecaphane-3,5-dione (Example 22);
Example 23: (R,E)-1⁷-(3-(dimethylamino)pyrrolidine-1-carbonyl)-1²,1³-dihydro-1¹H-2,6-diaza-1(2,1)-benzo[d]imidazola-4(1,3)-benzenacycloundecaphane-3,5-dione (Example 23);
(S,E)-1⁷-(2,4-dimethylpiperazine-1-carbonyl)-1²,1³-dihydro-1¹H-2,6-diaza-1(2,1)-benzo[d]imidazola-4(1,3)-benzenacycloundecaphane-3,5-dione (Example 24);
(E)-1⁷-((1R,4R)-5-methyl-2,5-diazabicyclo[2.2.1]heptane-2-carbonyl)-1²,1³-dihydro-1¹H-2,6-diaza-1(2,1)-benzo[d]imidazola-4(1,3)-benzenacycloundecaphane-3,5-dione (Example 25);
(E)-1⁷-(4-methyl-1,4-diazepane-1-carbonyl)-1²,1³-dihydro-1¹H-2,6-diaza-1(2,1)-benzo[d]imidazola-4(1,3)-benzenacycloundecaphane-3,5-dione (Example 26);
(E)-N,N-dimethyl-1⁵-((4-methylpiperazin-1-yl)methyl)-3,5-dioxo-1²,1³-dihydro-1¹H-2,6-diaza-1(2,1)-benzo[d]imidazola-4(1,3)-benzenacycloundecaphane-1⁷-carboxamide (Example 27);
(R, E)-11-methyl-1⁷-((1R,4R)-5-methyl-2,5-diazabicyclo[2.2.1]heptane-2-carbonyl)-1²,1³-dihydro-1¹H-2,6-diaza-1(2,1)-benzo[d]imidazola-4(1,3)-benzenacycloundecaphane-3,5-dione (Example 29);
(S, E)-11-methyl-1⁷-((1R,4R)-5-methyl-2,5-diazabicyclo[2.2.1]heptane-2-carbonyl)-1²,1³-dihydro-1¹H-2,6-diaza-1(2,1)-benzo[d]imidazola-4(1,3)-benzenacycloundecaphane-3,5-dione (Example 30);
(E)-N,N,11-trimethyl-3,5-dioxo-1²,1³-dihydro-1¹H-2,6-diaza-1(2,1)-benzo[d]imidazola-4(1,3)-benzenacycloundecaphane-1⁷-carboxamide (Example 31 and 32);
(E)-3-(11-methyl-3,5-dioxo-1²,1³-dihydro-1¹H-2,6-diaza-1(2,1)-benzo[d]imidazola-4(1,3)-benzenacycloundecaphane-1⁷-yl)pyridine 1-oxide (Example 33);
(E)-N,11-dimethyl-3,5-dioxo-1²,1³-dihydro-1¹H-2,6-diaza-1(2,1)-benzo[d]imidazola-4(1,3)-benzenacycloundecaphane-1⁷-carboxamide (Example 34);
(E)-11-methyl-1⁷-(2-oxo-1,2-dihydropyridin-3-yl)-1²,1³-dihydro-1¹H-2,6-diaza-1(2,1)-benzo[d]imidazola-4(1,3)-benzenacycloundecaphane-3,5-dione (Example 35 and 36);
(E)-1⁷-(3,3-difluoropyrrolidine-1-carbonyl)-11-methyl-1²,1³-dihydro-1¹H-2,6-diaza-1(2,1)-benzo[d]imidazola-4(1,3)-benzenacycloundecaphane-3,5-dione (Example 37);
methyl (E)-N-methyl-N-(11-methyl-3,5-dioxo-1²,1³-dihydro-1¹H-2,6-diaza-1(2,1)-benzo[d]imidazola-4(1,3)-benzenacycloundecaphane-1⁷-carbonyl)glycinate (Example 38);
(E)-N-cyclobutyl-N,11-dimethyl-3,5-dioxo-1²,1³-dihydro-1¹H-2,6-diaza-1(2,1)-benzo[d]imidazola-4(1,3)-benzenacycloundecaphane-1⁷-carboxamide (Example 39);
(E)-1⁷-(3,3-difluoroazetidine-1-carbonyl)-11-methyl-1²,1³-dihydro-1¹H-2,6-diaza-1(2,1)-benzo[d]imidazola-4(1,3)-benzenacycloundecaphane-3,5-dione (Example 40);
(E)-N-(3-hydroxycyclobutyl)-N,11-dimethyl-3,5-dioxo-1²,1³-dihydro-1¹H-2,6-diaza-1(2,1)-benzo[d]imidazola-4(1,3)-benzenacycloundecaphane-1⁷-carboxamide (Example 41);
(E)-11-methyl-1⁷-(2-oxa-6-azaspiro[3.3]heptane-6-carbonyl)-1²,1³-dihydro-1¹H-2,6-diaza-1(2,1)-benzo[d]imidazola-4(1,3)-benzenacycloundecaphane-3,5-dione (Example 42);
(E)-1⁷-(azetidine-1-carbonyl)-11-methyl-1²,1³-dihydro-1¹H-2,6-diaza-1(2,1)-benzo[d]imidazola-4(1,3)-benzenacycloundecaphane-3,5-dione (Example 43);
(E)-N-(11-methyl-3,5-dioxo-1²,1³-dihydro-1¹H-2,6-diaza-1(2,1)-benzo[d]imidazola-4(1,3)-benzenacycloundecaphane-1⁷-yl)acetamide (Example 45);
(R,E)-N,N,11-trimethyl-3,5-dioxo-1²,1³-dihydro-1¹H-2,6-diaza-1(2,1)-benzo[d]imidazola-4(1,3)-benzenacycloundecaphane-1⁷-sulfonamide (Example 46);
(E)-N-isopropyl-N,11-dimethyl-3,5-dioxo-1²,1³-dihydro-1¹H-2,6-diaza-1(2,1)-benzo[d]imidazola-4(1,3)-benzenacycloundecaphane-1⁷-carboxamide (Example 47 and 48);
(E)-1⁷-(3-methoxy-3-methylazetidine-1-carbonyl)-11-methyl-1²,1³-dihydro-1¹H-2,6-diaza-1(2,1)-benzo[d]imidazola-4(1,3)-benzenacycloundecaphane-3,5-dione (Example 49 and 50);
(S,E)-1⁶-chloro-N,N,11-trimethyl-3,5-dioxo-1²,1³-dihydro-1¹H-2,6-diaza-1(2,1)-benzo[d]imidazola-4(1,3)-benzenacycloundecaphane-1⁷-carboxamide (Example 53 and 54);
(R,E)-1⁶-chloro-N,N,11-trimethyl-3,5-dioxo-1²,1³-dihydro-1¹H-2,6-diaza-1(2,1)-benzo[d]imidazola-4(1,3)-benzenacycloundecaphane-1⁷-carboxamide (Example 55 and 56);
(R,E)-1⁷-(4,5-dimethyl-4H-1,2,4-triazol-3-yl)-11-methyl-1²,1³-dihydro-1¹H-2,6-diaza-1(2,1)-benzo[d]imidazola-4(1,3)-benzenacycloundecaphane-3,5-dione (Example 57);
(E)-11-methyl-1⁷-(2-oxopiperidin-3-yl)-1²,1³-dihydro-1¹H-2,6-diaza-1(2,1)-benzo[d]imidazola-4(1,3)-benzenacycloundecaphane-3,5-dione (Examples 58 to 61);
(E)-11-methyl-1⁷-(2-oxo-1,2-dihydropyridin-4-yl)-1²,1³-dihydro-1¹H-2,6-diaza-1(2,1)-benzo[d]imidazola-4(1,3)-benzenacycloundecaphane-3,5-dione (Example 62 and 63);
(R,E)-11-methyl-N,N-bis(methyl-d₃)-3,5-dioxo-1²,1³-dihydro-1¹H-2,6-diaza-1(2,1)-benzo[d]imidazola-4(1,3)-benzenacycloundecaphane-1⁷-carboxamide (Example 64);
(R,E)-11-methyl-1⁷-(3-methyl-2-oxoimidazolidin-1-yl)-1²,1³-dihydro-1¹H-2,6-diaza-1(2,1)-benzo[d]imidazola-4(1,3)-benzenacycloundecaphane-3,5-dione (Example 65);
(R,E)-11-methyl-1⁷-(1-methyl-1H-1,2,3-triazol-4-yl)-1²,1³-dihydro-1¹H-2,6-diaza-1(2,1)-benzo[d]imidazola-4(1,3)-benzenacycloundecaphane-3,5-dione (Example 66);
(E)-11-ethyl-N,N-dimethyl-3,5-dioxo-1²,1³-dihydro-1¹H-2,6-diaza-1(2,1)-benzo[d]imidazola-4(1,3)-benzenacycloundecaphane-1⁷-carboxamide (Example 67 and 68);
(R,E)-1⁵-(2-hydroxypropan-2-yl)-N,N,11-trimethyl-3,5-dioxo-1²,1³-dihydro-1¹H-2,6-diaza-1(2,1)-benzo[d]imidazola-4(1,3)-benzenacycloundecaphane-1⁷-carboxamide (Example 69);
(E)-N-(2-fluoroethyl)-N,11-dimethyl-3,5-dioxo-1²,1³-dihydro-1¹H-2,6-diaza-1(2,1)-benzo[d]imidazola-4(1,3)-benzenacycloundecaphane-1⁷-carboxamide (Example 70 and 71);
(E)-N-(2,2-difluoroethyl)-N,11-dimethyl-3,5-dioxo-1²,1³-dihydro-1¹H-2,6-diaza-1(2,1)-benzo[d]imidazola-4(1,3)-benzenacycloundecaphane-1⁷-carboxamide (Example 72 and 73);
(S,E)-11-methyl-1⁷-(3-methyl-2-oxoimidazolidin-1-yl)-1²,1³-dihydro-1¹H-2,6-diaza-1(2,1)-benzo[d]imidazola-4(1,3)-benzenacycloundecaphane-3,5-dione (Example 74);
(E)-11-methyl-1⁷-((S)-3-methylmorpholine-4-carbonyl)-1²,1³-dihydro-1¹H-2,6-diaza-1(2,1)-benzo[d]imidazola-4(1,3)-benzenacycloundecaphane-3,5-dione (Example 77 and 78);
(E)-11-methyl-1⁷-(1-oxa-6-azaspiro[3.3]heptane-6-carbonyl)-1²,1³-dihydro-1¹H-2,6-diaza-1(2,1)-benzo[d]imidazola-4(1,3)-benzenacycloundecaphane-3,5-dione (Example 79 and 80);
(E)-1⁷-(3-methoxyazetidine-1-carbonyl)-11-methyl-1²,1³-dihydro-1¹H-2,6-diaza-1(2,1)-benzo[d]imidazola-4(1,3)-benzenacycloundecaphane-3,5-dione (Example 81 and 82);
(R,E)-11-methyl-1⁷-(1-methyl-1H-1,2,4-triazol-3-yl)-1²,1³-dihydro-1¹H-2,6-diaza-1(2,1)-benzo[d]imidazola-4(1,3)-benzenacycloundecaphane-3,5-dione (Example 83 and 84)
(R,E)-11-methyl-1⁷-(1-methyl-1H-1,2,4-triazol-5-yl)-1²,1³-dihydro-1¹H-2,6-diaza-1(2,1)-benzo[d]imidazola-4(1,3)-benzenacycloundecaphane-3,5-dione (Example 85 and 86);
(S,E)-11-methyl-1⁷-(1-methyl-1H-1,2,3-triazol-4-yl)-1²,1³-dihydro-1¹H-2,6-diaza-1(2,1)-benzo[d]imidazola-4(1,3)-benzenacycloundecaphane-3,5-dione (Example 87);
(E)-1⁷-((S)-3-fluoropyrrolidine-1-carbonyl)-11-methyl-1²,1³-dihydro-1¹H-2,6-diaza-1(2,1)-benzo[d]imidazola-4(1,3)-benzenacycloundecaphane-3,5-dione (Example 88 and 89);
(E)-11-methyl-1⁷-(1-methyl-1H-imidazol-2-yl)-1²,1³-dihydro-1¹H-2,6-diaza-1(2,1)-benzo[d]imidazola-4(1,3)-benzenacycloundecaphane-3,5-dione (Example 90 and 91);
(E)-1⁷-((R)-3-fluoropyrrolidine-1-carbonyl)-11-methyl-1²,1³-dihydro-1¹H-2,6-diaza-1(2,1)-benzo[d]imidazola-4(1,3)-benzenacycloundecaphane-3,5-dione (Example 92 and 93);
(R,E)-1⁷-(3-hydroxy-3-methylazetidine-1-carbonyl)-11-methyl-1²,1³-dihydro-1¹H-2,6-diaza-1(2,1)-benzo[d]imidazola-4(1,3)-benzenacycloundecaphane-3,5-dione (Example 94);
(S,E)-1⁷-(3-hydroxy-3-methylazetidine-1-carbonyl)-11-methyl-1²,1³-dihydro-1¹H-2,6-diaza-1(2,1)-benzo[d]imidazola-4(1,3)-benzenacycloundecaphane-3,5-dione (Example 95);
(E)-1⁶-(4-methylpiperazine-1-carbonyl)-1²,1³-dihydro-1¹H-2,6-diaza-1(2,1)-benzo[d]imidazola-4(1,3)-benzenacycloundecaphane-3,5-dione (Example 97);
(E)-1⁶-(4-(oxetan-3-yl)piperazine-1-carbonyl)-1²,1³-dihydro-1¹H-2,6-diaza-1(2,1)-benzo[d]imidazola-4(1,3)-benzenacycloundecaphane-3,5-dione (Example 98);
(E)-1⁵-fluoro-1²,1³-dihydro-1¹H-2,6-diaza-1(2,1)-benzo[d]imidazola-4(1,3)-benzenacycloundecaphane-3,5-dione (Example 99);
(E)-1⁶-(piperazine-1-carbonyl)-1²,1³-dihydro-1¹H-2,6-diaza-1(2,1)-benzo[d]imidazola-4(1,3)-benzenacycloundecaphane-3,5-dione (Example 100);
(E)-1⁶-((4-methylpiperazin-1-yl)methyl)-1²,1³-dihydro-1¹H-2,6-diaza-1(2,1)-benzo[d]imidazola-4(1,3)-benzenacycloundecaphane-3,5-dione (Example 101);
(E)-1⁵-((4-methylpiperazin-1-yl)methyl)-1²,1³-dihydro-1¹H-2,6-diaza-1(2,1)-benzo[d]imidazola-4(2,6)-pyridinacycloundecaphane-3,5-dione (Example 102);
(E)-1⁵-((4-methylpiperazin-1-yl)methyl)-1²,1³-dihydro-1¹H-2,6-diaza-1(2,1)-benzo[d]imidazola-4(2,6)-pyridinacycloundecaphane-3,5-dione (Example 103);
(1²E,4²Z)-1⁵-((4-methylpiperazin-1-yl)methyl)-1²,1³-dihydro-1¹H-2,6-diaza-1(2,1)-benzo[d]imidazola-4(4,2)-thiophenacycloundecaphane-3,5-dione (Example 104);
(E)-4⁶-fluoro-1⁵-((4-methylpiperazin-1-yl)methyl)-1²,1³-dihydro-1¹H-2,6-diaza-1(2,1)-benzo[d]imidazola-4(1,3)-benzenacycloundecaphane-3,5-dione (Example 105);
(S,E)-1⁷-((R)-3-hydroxy-3-methylpyrrolidine-1-carbonyl)-11-methyl-1²,1³-dihydro-1¹H-2,6-diaza-1(2,1)-benzo[d]imidazola-4(1,3)-benzenacycloundecaphane-3,5-dione (Example 106);
(S,E)-1⁷-(3-cyclopropoxyazetidine-1-carbonyl)-11-methyl-1²,1³-dihydro-1¹H-2,6-diaza-1(2,1)-benzo[d]imidazola-4(1,3)-benzenacycloundecaphane-3,5-dione (Example 107);
(1²Z,4²E)-N,N,5-trimethyl-2,11-dioxo-4²,4³-dihydro-4¹H-3,10-diaza-1(4,2)-thiazola-4(2,1)-benzo[d]imidazolacycloundecaphane-4⁷-carboxamide (Example 108 and 109);
(S,E)-11-methyl-1⁷-(3-(trifluoromethoxy)azetidine-1-carbonyl)-1²,1³-dihydro-11H-2,6-diaza-1(2,1)-benzo[d]imidazola-4(1,3)-benzenacycloundecaphane-3,5-dione (Example 110);
(S,E)-1⁷-((1S,4S)-2-oxa-5-azabicyclo[2.2.1]heptane-5-carbonyl)-11-methyl-1²,1³-dihydro-1¹H-2,6-diaza-1(2,1)-benzo[d]imidazola-4(1,3)-benzenacycloundecaphane-3,5-dione (Example 111);
(S,E)-1⁷-((1R,4R)-2-oxa-5-azabicyclo[2.2.1]heptane-5-carbonyl)-11-methyl-1²,1³-dihydro-1¹H-2,6-diaza-1(2,1)-benzo[d]imidazola-4(1,3)-benzenacycloundecaphane-3,5-dione (Example 112);
(S,E)-11-methyl-1⁷-(2-oxopyrrolidin-1-yl)-1²,1³-dihydro-1¹H-2,6-diaza-1(2,1)-benzo[d]imidazola-4(1,3)-benzenacycloundecaphane-3,5-dione (Example 113);
(S,E)-11-methyl-1⁷-(2-oxa-6-azaspiro[3.3]heptane-6-carbonyl)-1²,1³-dihydro-1¹H-2,6-diaza-1(2,1)-benzo[d]imidazola-4(1,3)-benzenacycloundecaphane-3,5-dione (Example 114);
(S,E)-11-methyl-1⁷-(2-oxa-6-azaspiro[3.4]octane-6-carbonyl)-1²,1³-dihydro-1¹H-2,6-diaza-1(2,1)-benzo[d]imidazola-4(1,3)-benzenacycloundecaphane-3,5-dione (Example 115);
(S,E)-11-methyl-1⁷-(5-oxa-2-azaspiro[3.4]octane-2-carbonyl)-1²,1³-dihydro-1¹H-2,6-diaza-1(2,1)-benzo[d]imidazola-4(1,3)-benzenacycloundecaphane-3,5-dione (Example 116);
(S,E)-1⁷-(7,7-difluoro-5-oxa-2-azaspiro[3.4]octane-2-carbonyl)-11-methyl-1²,1³-dihydro-1¹H-2,6-diaza-1(2,1)-benzo[d]imidazola-4(1,3)-benzenacycloundecaphane-3,5-dione (Example 117);
(S,E)-11-methyl-N,N-bis(methyl-d₃)-3,5-dioxo-1²,1³-dihydro-1¹H-2,6-diaza-1(2,1)-benzo[d]imidazola-4(1,3)-benzenacycloundecaphane-1⁷-carboxamide (Example 118); and
(S,E)-11-methyl-1⁴-(2-oxopyrrolidin-1-yl)-1²,1³-dihydro-1¹H-2,6-diaza-1(2,3)-imidazo[4,5-c]pyridina-4(1,3)-benzenacycloundecaphane-3,5-dione (Example 119).

Particularly preferred compounds include:
(R, E)-11-methyl-1⁷-((1R,4R)-5-methyl-2,5-diazabicyclo[2.2.1]heptane-2-carbonyl)-1²,1³-dihydro-1¹H-2,6-diaza-1(2,1)-benzo[d]imidazola-4(1,3)-benzenacycloundecaphane-3,5-dione (Example 29);
(S, E)-11-methyl-1⁷-((1R,4R)-5-methyl-2,5-diazabicyclo[2.2.1]heptane-2-carbonyl)-1²,1³-dihydro-1¹H-2,6-diaza-1(2,1)-benzo[d]imidazola-4(1,3)-benzenacycloundecaphane-3,5-dione (Example 30);
(S,E)-11-methyl-1⁷-(1-methyl-1H-1,2,3-triazol-4-yl)-1²,1³-dihydro-1¹H-2,6-diaza-1(2,1)-benzo[d]imidazola-4(1,3)-benzenacycloundecaphane-3,5-dione (Example 87);
(S,E)-11-methyl-1⁷-(2-oxopyrrolidin-1-yl)-1²,1³-dihydro-1¹H-2,6-diaza-1(2,1)-benzo[d]imidazola-4(1,3)-benzenacycloundecaphane-3,5-dione (Example 113);
(S,E)-11-methyl-N,N-bis(methyl-d₃)-3,5-dioxo-1²,1³-dihydro-1¹H-2,6-diaza-1(2,1)-benzo[d]imidazola-4(1,3)-benzenacycloundecaphane-1⁷-carboxamide (Example 118);
and
(S,E)-11-methyl-1⁴-(2-oxopyrrolidin-1-yl)-1²,1³-dihydro-1¹H-2,6-diaza-1(2,3)-imidazo[4,5-c]pyridina-4(1,3)-benzenacycloundecaphane-3,5-dione (Example 119).

Further preferred compounds that can be mentioned are:
1⁵-((4-methylpiperazin-1-yl)methyl)-1¹H-2,6-diaza-1(2,1)-benzo[d]imidazola-4(1,3)-benzenacycloundecaphane-3,5-dione (Example 3);
1¹-methyl-1⁷-(4-methylpiperazine-1-carbonyl)-1¹H-2,6-diaza-1(2,1)-benzo[d]imidazola-4(1,3)-benzenacycloundecaphane-3,5-dione (Example 8);
1⁷-(4-methylpiperazine-1-carbonyl)-1¹H-2,6-diaza-1(2,1)-benzo[d]imidazola-4(1,3)-benzenacycloundecaphane-3,5-dione (Example 10);

For the avoidance of doubt, when a compound structure is depicted or named herein, the compound structure or name is considered to encompass all stereoisomers of the compound, unless stated otherwise. For example, if the stereochemistry of the compound is not indicated in the compound structure or name, the compound structure or name encompass all stereoisomers of the compound.

Also for the avoidance of doubt, when a compound structure is depicted or named herein, the compound structure or name is considered to encompass all tautomeric forms of the compound, unless stated otherwise. For example, compounds according to formula (Ie) below are encompassed by formula (I):

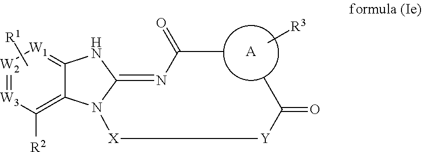

In certain embodiments, the compound of formula (I) is according to formula (If)

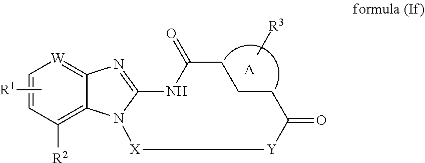

or a tautomer thereof,
wherein,
A is a 6 membered aromatic ring or 5 to 6 membered heteroaromatic ring comprising 1 or 2 atoms selected from the group consisting of N, S and O;
W is CH or N;
X is linear C₂₋₈alkylene, optionally substituted with one or more groups independently selected from halogen, OH and C₁₋₆alkyl and/or wherein optionally one or more carbon atoms in the alkylene is replaced by an atom independently selected from O, N and S;
Y is NH or O;
R¹ and R² are independently selected from H; C₁₋₆alkyl; C₂₋₆alkenyl; C₂₋₆alkynyl; OC₁₋₆alkyl; OC₂₋₆alkenyl; OC₂₋₆alkynyl; —R^{a}, C(O)R^{a}; C(O)OR^{a}; OCR^{a}; OC(O)(R^{a}); —C₁₋₆ alkylene-R^{a}; —C(O)C₁₋₆alkylene-R^{a}; —C(O)OC₁₋₆alkylene-R^{a}; —OC₁₋₆alkylene-R^{a}; OC(O)C₁₋₆alkylene-R^{a}; and —R^{b}, wherein said alkyl, alkenyl or alkynyl is optionally substituted with one or more R^{b} groups;
R³ is H, halogen, OH, C₁₋₆alkyl or OC₁₋₆alkyl, wherein said alkyl is optionally substituted with one or more halogen;
   wherein,
R^{a} is selected from 3 to 8 membered non-aromatic carbocycle; 3 to 8 membered heterocycle or 6 to 12 membered spirocyclic heterocycle comprising one, two or three atoms selected from the group consisting of N, S and O; said non-aromatic carbocycle, heterocycle or spirocyclic heterocycle optionally being bridged and/or substituted with one or more groups independently selected from halogen; OH; O; SH; NH₂; NHC₁₋₄alkyl; N(C₁₋₄alkyl)₂; C₁₋₄alkyl; 3 to 6 membered heterocycle comprising one, two or three atoms selected from the group consisting of N, S and O; OC₁₋₄alkyl; C(O)C₁₋₄alkyl; C(O)OC₁₋₄alkyl; OC(O)C₁₋₄alkyl; C(O)NH₂; —C₁₋₄alkylene-OC₁₋₄alkyl; or —C₁₋₄alkylene-OH; and
Each R^{b} is independently selected from C(O)R^{c}; C(O)OR^{c}; OC(O)R^{c}; SR^{c}; SO₂R^{c}; NO₂; CN; NR^{c}R^{d}; N(R^{c})SO₂C₁₋₃alkyl; N(R^{c})C(O)R^{d}; C(O)NR^{c}R^{d}; SO₂NR^{c}R^{d}; halogen; SH; NH₂; and OH; said alkyl optionally being substituted with one or more groups independently selected from halogen, SH, NH₂ and OH and/or wherein optionally one or more carbon atoms in the alkyl is replaced by an atom independently selected from O, N and S; and
R^{c} and R^{d} are independently selected from H; C₁₋₆alkyl; C₂₋₆alkenyl; C₂₋₆alkynyl; —C₁₋₄alkylene-OC₁₋₄alkyl; and 3 to 8 membered non-aromatic carbocycle; said alkyl, alkenyl, alkynyl or carbocycle optionally being substituted with one or more groups selected from halogen, SH, NH₂, OH and ═O, and/or wherein optionally one or more carbon atoms in the alkyl is replaced by an atom independently selected from O, N and S;
or a pharmaceutically acceptable salt or solvate thereof.

For the avoidance of doubt, in this document, it is intended that compounds of formula (I) include all tautomeric forms, salts and solvates thereof, unless stated otherwise.

Salts of compounds of formula (I), which are suitable for use in the present invention, are those wherein a counterion is pharmaceutically acceptable. However, salts having non-pharmaceutically acceptable counter-ions are within the scope of the present invention, for example, for use as intermediates in the preparation of the compounds of formula (I) and their pharmaceutically acceptable salts.

Suitable salts for use according to the invention include those formed with organic or inorganic acids. In particular, suitable salts formed with acids according to the invention include those formed with mineral acids, strong organic carboxylic acids, such as alkanecarboxylic acids of 1 to 4 carbon atoms which are unsubstituted or substituted, for example, by halogen, such as saturated or unsaturated dicarboxylic acids, such as hydroxycarboxylic acids, such as amino acids, or with organic sulfonic acids, such as (C₁-C₄)-alkyl- or aryl-sulfonic acids which are unsubstituted or substituted, for example by halogen. Pharmaceutically acceptable acid addition salts include those formed from hydrochloric, hydrobromic, sulphuric, nitric, citric, tartaric, acetic, phosphoric, lactic, pyruvic, acetic, trifluoroacetic, succinic, perchloric, fumaric, maleic, glycolic, lactic, salicylic, oxalic, oxaloacetic, methanesulfonic, ethanesulfonic, p-toluenesulfonic, formic, benzoic, malonic, naphthalene-2-sulfonic, benzenesulfonic, isethionic, ascorbic, malic, phthalic, aspartic, and glutamic acids, lysine and arginine. Suitable cations which may be present in salts include alkali metal cations, especially sodium, potassium and calcium, and ammonium or amino cations.

Pharmaceutically acceptable base salts include ammonium salts, alkali metal salts, for example those of potassium and sodium, alkaline earth metal salts, for example those of calcium and magnesium, and salts with organic bases, for example dicyclohexylamine, N-methyl-D-glucomine, morpholine, thiomorpholine, piperidine, pyrrolidine, a mono-, di- or tri-lower alkylamine, for example ethyl-, tert-butyl-, diethyl-, diisopropyl-, triethyl-, tributyl- or dimethyl-propylamine, or a mono-, di- or trihydroxy lower alkylamine, for example mono-, di- or triethanolamine. Corresponding internal salts may furthermore be formed.

Those skilled in the art of organic chemistry will appreciate that many organic compounds can form complexes with solvents in which they are reacted or from which they are precipitated or crystallized. These complexes are known as “solvates”. For example, a complex with water is known as a “hydrate”. The complex may incorporate a solvent in stoichiometric or non-stoichiometric amounts. Solvates are described in Water-Insoluble Drug Formulation, 2^{nd} ed R. Lui CRC Press, page 553 and Byrn et al Pharm Res 12(7), 1995, 945-954. Before it is made up in solution, the compound of formula (I) may be in the form of a solvate. Solvates of compounds of formula (I) which are suitable for use as a medicament according to the invention are those wherein the associated solvent is pharmaceutically acceptable. For example, a hydrate is a pharmaceutically acceptable solvate. However, solvates having non-pharmaceutically acceptable associated solvents may find use as intermediates in the preparation of the compounds of formula (I) and their pharmaceutically acceptable esters, amides, carbamates and/or salts thereof.

A compound which, upon administration to the recipient, is capable of being converted into a compound of formula (I), or an active metabolite or residue thereof, is known as a “prodrug”. Thus, in certain embodiments, the compound of formula (I) may be provided in the form of a prodrug. A prodrug may, for example, be converted within the body, e.g. by hydrolysis in the blood, into its active form that has medical effects. Pharmaceutical acceptable prodrugs are described in T. Higuchi and V. Stella, Prodrugs as Novel Delivery Systems, Vol. 14 of the A. C. S. Symposium Series (1976); “Design of Prodrugs” ed. H. Bundgaard, Elsevier, 1985; and in Edward B. Roche, ed., Bioreversible Carriers in Drug Design, American Pharmaceutical Association and Pergamon Press, 1987, which are incorporated herein by reference.

Compounds of formula (I) may have an appropriate group converted to an ester, an amide or a carbamate. Thus typical ester and amide groups formed from an acid group in the compound of the formula (I) include —COOR^{G}, —CONR^{G}₂, —SO₂OR^{G}, or —SO₂N(R^{G})₂, while typical ester and amide and carbamate groups formed from an —OH or —NHR^{G} group in the compound of the formula (I) include —OC(O)R^{G}, —NR^{G}C(O)R^{G}, —NR^{G}CO₂R^{G}, —OSO₂R^{G}, and —NR^{G}SO₂R^{G}, where R^{G} is selected from the group consisting of C₁₋₈alkyl, C₂₋₈alkenyl, C₂₋₈alkynyl, C₃₋₈cycloalkyl and C₃₋₈cycloalkylC₁₋₈alkyl, haloC₁₋₈alkyl, dihaloC₁₋₈alkyl, trihaloC₁₋₈alkyl, phenyl and phenylC₁₋₄alkyl; preferably R^{G} is selected from the group consisting of C₁₋₆alkyl, C₂₋₆alkenyl, C₂₋₆alkynyl, C₃₋₈cycloalkyl and C₃₋₈cycloalkylC₁₋₆alkyl; more preferably R^{G} is selected from the group consisting of C₁₋₄alkyl, C₂₋₄alkenyl, C₂₋₄alkynyl, C₃₋₈cycloalkyl and C₃₋₈cycloalkylC₁₋₄alkyl.

### Pharmaceutical Compositions

While it is possible for a compound of formula (I) to be administered alone, it is preferable for it to be present in a composition and particularly in a pharmaceutical composition. Pharmaceutical compositions of the present invention comprise a compound of formula (I) and one or more pharmaceutically acceptable excipient. Pharmaceutical compositions include those suitable for oral, parenteral (including subcutaneous, intradermal, intraosseous infusion, intramuscular, intravascular (bolus or infusion), and intramedullary), intraperitoneal, transmucosal, transdermal, rectal and topical (including dermal, buccal, sublingual and intraocular) administration, although the most suitable route may depend upon, for example, the type of disease or condition to be treated or prevented.

Pharmaceutical compositions of the present invention suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The compound of formula (I) may also be presented as a bolus, electuary or paste. Various pharmaceutically acceptable carriers and their formulation are described in standard formulation treatises, e.g., Remington's Pharmaceutical Sciences by E. W. Martin. See also Wang, Y. J. and Hanson, M. A., Journal of Parenteral Science and Technology, Technical Report No. 10, Supp. 42:2S, 1988.

Formulations for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilised) condition requiring only the addition of the sterile liquid carrier, for example saline or water-for-injection, immediately prior to use. Extemporaneous injection solutions and suspensions may be prepared from sterile powders, granules and tablets of the kind previously described. Exemplary compositions for parenteral administration include injectable solutions or suspensions which can contain, for example, suitable non-toxic, parenterally acceptable diluents or solvents, such as mannitol, 1,3-butanediol, water, Ringer's solution, an isotonic sodium chloride solution, or other suitable dispersing or wetting and suspending agents, including synthetic mono- or diglycerides, and fatty acids, including oleic acid, or Cremaphor.

Compositions for nasal, aerosol or inhalation administration include solutions in saline, which can contain, for example, benzyl alcohol or other suitable preservatives, absorption promoters to enhance bioavailability, and/or other solubilizing or dispersing agents such as those known in the art.

Formulations for rectal administration may be presented as a suppository with the usual carriers such as cocoa butter, synthetic glyceride esters or polyethylene glycol. Such carriers are typically solid at ordinary temperatures, but liquefy and/or dissolve in the rectal cavity to release the drug.

Formulations for topical administration in the mouth, for example buccally or sublingually, include lozenges comprising the active ingredient in a flavoured basis such as sucrose and acacia or tragacanth, and pastilles comprising the active ingredient in a basis such as gelatin and glycerine or sucrose and acacia. Exemplary compositions for topical administration include a topical carrier such as Plastibase (mineral oil gelled with polyethylene).

Preferred unit dosage compositions are those containing an exploratory dose or therapeutic dose, or an appropriate fraction thereof, of a compound of formula (I).

In preferred embodiments, a composition of the invention consists essentially of a compound of formula (I) and at least one pharmaceutically acceptable excipient.

It should be understood that in addition to the ingredients particularly mentioned above, the compositions for use in this invention may include other agents conventional in the art having regard to the type of composition in question.

The compositions of the invention may comprise one or more further therapeutic agents. Examples of further therapeutic agents that may be present in a composition of the present invention include, but not limited to one or more Hsp90 inhibitors (e.g., 2-(((1r,4r)-4-hydroxycyclohexyl)amino)-4-(3,6,6-trimethyl-4-oxo-4,5,6,7-tetrahydro-1H-indazol-1-yl)benzamide), one or more Hsp70 inhibitors, one or more MEK, BRAF or RAF inhibitors or one or more further other an anti-cancer/chemo-therapeutic agents.

### Treatments

The compounds of formula (I), and compositions of the invention, find use as medicaments. In particular, compounds and compositions of the present invention find use in the treatment and/or prophylaxis of cancers, inflammatory diseases, autoinflammatory diseases and autoimmune diseases. Additionally, or alternatively, compounds and compositions of the invention find use in the treatment or prevention of diseases or disorders that are associated with aberrant TAK1 activity. Aberrant TAK1 activity may arise, for example due to the presence of a gain-of-function mutations in the TAK1 pathway. For example frontometaphyseal dysplasia type 2 (FMD2) may be caused by a mutation in the MAP3K7 gene. Additionally, or alternatively, compounds and compositions of the invention find use in the treatment or prophylaxis of a disease or disorder that may be treated or prevented by inhibition of TAK1.

Cancer that may be treated and/or prevented by administering a compound or composition of the invention include, but are not limited to, those associated with a mutation of the KRAS gene, particularly cancers classed as KRAS-dependent cancers. Such cancers include cancers that are classed as refractory, relapsed or refractory-relapsed, particularly refractory cancers.

Conditions that may be treated and/or prevented by administering a compound or composition of the invention include, but are not limited to, diseases caused by deficiency, lack of CYLD (cylindromatosis) or mutations in the CYLD gene including Nonalcoholic Fatty Liver Disease (NAFLD) and nonalcoholic steatohepatitis (NASH) and Brooke-Spiegler syndrome, multiple familial trichoepithelioma, and familial cylindromatosis.

Particular examples of cancers that may be treated or prevented by administering a compound or composition of the invention include carcinoma, a sarcoma, a myeloma, a leukemia, a lymphoma or a mixed type of cancer. Further exemplary cancers that may be treated or prevented by administering a compound or composition of the invention include, but not limited to, digestive/gastrointestinal cancers such as anal cancer; bile duct cancer; extrahepatic bile duct cancer; appendix cancer; carcinoid tumor, gastrointestinal cancer; colon cancer; colorectal cancer including childhood colorectal cancer; esophageal cancer including childhood esophageal cancer; gallbladder cancer; gastric (stomach) cancer including childhood gastric (stomach) cancer; hepatocellular (liver) cancer including adult (primary) hepatocellular (liver) cancer and childhood (primary) hepatocellular (liver) cancer; pancreatic cancer including childhood pancreatic cancer; sarcoma, rhabdomyosarcoma; islet cell pancreatic cancer; rectal cancer; and small intestine cancer; endocrine cancers such as islet cell carcinoma (endocrine pancreas); adrenocortical carcinoma including childhood adrenocortical carcinoma; gastrointestinal carcinoid tumor; parathyroid cancer; pheochromocytoma; pituitary tumor; thyroid cancer including childhood thyroid cancer; childhood multiple endocrine neoplasia syndrome; and childhood carcinoid tumor; eye cancers such as intraocular melanoma; and retinoblastoma; musculoskeletal cancers such as Ewing's family of tumors; osteosarcoma/malignant fibrous histiocytoma of the bone; childhood rhabdomyosarcoma; soft tissue sarcoma including adult and childhood soft tissue sarcoma; clear cell sarcoma of tendon sheaths; and uterine sarcoma; breast cancer such as breast cancer including childhood and male breast cancer and breast cancer in pregnancy; neurologic cancers such as childhood brain stemglioma; brain tumor; childhood cerebellar astrocytoma; childhood cerebral astrocytoma/malignant glioma; childhood ependymoma; childhood medulloblastoma; childhood pineal and supratentorial primitive neuroectodermal tumors; childhood visual pathway and hypothalamic glioma; other childhood brain cancers; adrenocortical carcinoma; central nervous system lymphoma, primary; childhood cerebellar astrocytoma; neuroblastoma; craniopharyngioma; spinal cord tumors; central nervous system atypical teratoid/rhabdoid tumor; central nervous system embryonal tumors; and childhood supratentorial primitive neuroectodermal tumors and pituitary tumor; genitourinary cancers such as bladder cancer including childhood bladder cancer; renal cell (kidney) cancer; ovarian cancer including childhood ovarian cancer; ovarian epithelial cancer; ovarian low malignant potential tumor; penile cancer; prostate cancer; renal cell cancer including childhood renal cell cancer; renal pelvis and ureter, transitional cell cancer; testicular cancer; urethral cancer; vaginal cancer; vulvar cancer; cervical cancer; Wilms tumor and other childhood kidney tumors; endometrial cancer; and gestational trophoblastic tumor; Germ cell cancers such as childhood extracranial germ cell tumor; extragonadal germ cell tumor; ovarian germ cell tumor; head and neck cancers such as lip and oral cavity cancer; oral cancer including childhood oral cancer (e.g., oral squamous cell carcinoma); hypopharyngeal cancer; laryngeal cancer including childhood laryngeal cancer; metastatic squamous neck cancer with occult primary; mouth cancer; nasal cavity and paranasal sinus cancer; nasopharyngeal cancer including childhood nasopharyngeal cancer; oropharyngeal cancer; parathyroid cancer; pharyngeal cancer; salivary gland cancer including childhood salivary gland cancer; throat cancer; and thyroid cancer; Hematologic/blood cell cancers such as a leukemia (e.g., acute lymphoblastic leukemia including adult and childhood acute lymphoblastic leukemia; acute myeloid leukemia including adult and childhood acute myeloid leukemia; chronic lymphocytic leukemia such as B Cell chronic lymphocytic leukemia; chronic myelogenous leukemia; and hairy cell leukemia); a lymphoma (e.g., AIDS-related lymphoma; cutaneous T-cell lymphoma; Hodgkin's lymphoma including adult and childhood Hodgkin's lymphoma and Hodgkin's lymphoma during pregnancy; non-Hodgkin's lymphoma including adult and childhood non-Hodgkin's lymphoma and non-Hodgkin's lymphoma during pregnancy; mycosis fungoides; Sezary syndrome; Waldenstrom's macroglobulinemia; primary mediastinal large B cell lymphoma; mantle cell lymphoma; diffuse large B cell lymphoma; and primary central nervous system lymphoma); and other hematologic cancers (e.g., chronic myeloproliferative disorders; multiple myeloma/plasma cell neoplasm; myelodysplastic syndromes; and myelodysplastic/myeloproliferative disorders); lung cancer such as non-small cell lung cancer; and small cell lung cancer; respiratory cancers such as adult malignant mesothelioma; childhood malignant mesothelioma; malignant thymoma; childhood thymoma; thymic carcinoma; bronchial adenomas/carcinoids including childhood bronchial adenomas/carcinoids; pleuropulmonary blastoma; non-small cell lung cancer; and small cell lung cancer; skin cancers such as Kaposi's sarcoma; Merkel cell carcinoma; melanoma; and childhood skin cancer; AIDS-related malignancies; other childhood cancers, unusual cancers of childhood and cancers of unknown primary site; and metastases of the aforementioned cancers. Yet further examples of cancers include breast cancer, colorectal cancer, leukemia, neurofibrodomas, or non-small cell lung cancer or a combination thereof.

Inflammatory diseases, autoinflammatory diseases and autoimmune diseases that may be treated or prevented by administering a compound or composition of the invention include systemic lupus erythematosus (SLE), Sjögren's syndrome, rheumatoid arthritis, osteoarthritis, gout, psoriatic arthritis, psoriasis, ankylosing spondylitis, diabetes mellitus, malaria, Crohn's disease, ulcerative colitis, inflammatory bowel disease, Familial Mediterranean Fever (FMF), Cryopyrin-associated periodic syndromes (CAPS), Deficiency of IL-1-Receptor Antagonist (DIRA) and Hyper IgD Syndrome (HIDS), myocardial infarction, reperfusion, ischemia or stroke. Multiple sclerosis and systemic sclerosis may also be mentioned. Lung conditions including idiopathic pulmonary fibrosis, asthma and chronic obstructive pulmonary disease (COPD) may also be treated or prevented by administering a compound or composition of the invention.

Further inflammatory diseases and conditions that may be treated or prevented by administering a compound or composition of the invention include ocular allergy, conjunctivitis, keratoconjunctivitis sicca, vernal conjunctivitis, allergic rhinitis, autoimmune hematological disorders, hemolytic anemia, aplastic anemia, pure red cell anemia, idiopathic thrombocytopenia, polychondritis, scleroderma, Wegener granulamatosis, dermatomyositis, chronic active hepatitis, myasthenia gravis, Steven-Johnson syndrome, idiopathic sprue, autoimmune inflammatory bowel disease, irritable bowel syndrome, celiac disease, periodontitis, hyaline membrane disease, kidney disease, glomerular disease, alcoholic liver disease, endocrine opthalmopathy, Grave's disease, sarcoidosis, alveolitis, chronic hypersensitivity pneumonitis, primary biliary cirrhosis, uveitis (anterior or posterior), interstitial lung fibrosis, systemic juvenile idiopathic arthritis, nephritis, vasculitis, diverticulitis, interstitial cystitis, glomerulonephritis, idiopathic nephrotic syndrome, minimal change nephropathy, chronic granulomatous disease, endometriosis, leptospirosis renal disease, glaucoma, retinal disease, headache, pain, complex regional pain syndrome, cardiac hypertrophy, muscle wasting, catabolic disorders, obesity, fetal growth retardation, hypercholesterolemia, heart disease, chronic heart failure, mesothelioma, anhidrotic urticarial dysplasia, Behcet's disease, incontinentia pigmenti, Paget's disease, pancreatitis, hereditary periodic fever syndrome, acute lung injury, acute respiratory distress syndrome, eosinophilia, hypersensitivities, anaphylaxis, fibrositis, gastritis, gastroenteritis, nasal sinusitis, silica induced diseases, cystic fibrosis, acid-induced lung injury, pulmonary hypertension, polyneuropathy, cataracts, muscle inflammation in conjunction with systemic sclerosis, inclusion body myositis, thyroiditis, Addison's disease, lichen planus, appendicitis, atopic dermatitis, allergy, blepharitis, bronchiolitis, bronchitis, bursitis, cervicitis, cholangitis, cholecystitis, chronic graft rejection, colitis, cystitis, dacryoadenitis, dermatitis, juvenile rheumatoid arthritis, encephalitis, endocarditis, endometritis, enteritis, enterocolitis, epicondylitis, epididymitis, fasciitis, Henoch-Schonlein purpura, hepatitis, hidradenitis suppurativa, immunoglobulin A nephropathy, interstitial lung disease, laryngitis, mastitis, meningitis, myelitis myocarditis, myositis, oophoritis, orchitis, osteitis, otitis, parotitis, pericarditis, peritonitis, pharyngitis, urticaria, phlebitis, pneumonitis, pneumonia, polymyositis, proctitis, prostatitis, pyelonephritis, rhinitis, salpingitis, sinusitis, stomatitis, synovitis, tendonitis, tonsillitis, vulvitis, alopecia areata, erythema multiforma, dermatitis herpetiformis, vitiligo, hypersensitivity angiitis, bullous pemphigoid, pemphigus vulgaris, pemphigus foliaceus, paraneoplastic pemphigus, epidermolysis bullosa acquisita, acute or chronic gout, chronic gouty arthritis, and also autoimmune syndromes including Hashimoto's thyroiditis, type I diabetes, allergic encephalomyelitis, post-infectious autoimmune diseases including rheumatic fever and post-infectious glomerulonephritis, inflammatory and hyperproliferative skin diseases, contact dermatitis, eczematous dermatitis, seborrhoeic dermatitis, pemphigus, epidermolysis bullosa, angioedemas, erythema, cutaneous eosinophilia, lupus erythematosus, acne, keratoconjunctivitis, uveitis associated with Behcet's disease, keratitis, herpetic keratitis, conical cornea, dystrophia epithelialis corneae, corneal leukoma, ocular pemphigus, Mooren's ulcer, scleritis, Graves' opthalmopathy, Vogt-Koyanagi-Harada syndrome, pollen allergies, reversible obstructive airway disease, bronchial asthma, allergic asthma, intrinsic asthma, extrinsic asthma, dust asthma, chronic or inveterate asthma, late asthma and airway hyper-responsiveness, gastric ulcers, vascular damage caused by ischemic diseases and thrombosis, ischemic bowel diseases, necrotizing enterocolitis, intestinal lesions associated with thermal burns, coeliac diseases, eosinophilic gastroenteritis, mastocytosis, migraine, eczema, interstitial nephritis, Goodpasture's syndrome, hemolytic-uremic syndrome, diabetic nephropathy, multiple myositis, Guillain-Barre syndrome, Meniere's disease, polyneuritis, multiple neuritis, mononeuritis, radiculopathy, hyperthyroidism, Basedow's disease, pure red cell aplasia, hypoplastic anemia, idiopathic thrombocytopenic purpura, autoimmune hemolytic anemia, agranulocytosis, pernicious anemia, megaloblastic anemia, anerythroplasia, osteoporosis, fibroid lung, idiopathic interstitial pneumonia, leukoderma vulgaris, ichthyosis vulgaris, photoallergic sensitivity, cutaneous T cell lymphoma, chronic lymphocytic leukemia, arteriosclerosis, atherosclerosis, aortitis syndrome, polyarteritis nodosa, myocardosis, Wegener's granuloma, adiposis, eosinophilic fascitis, lesions of gingiva, periodontium, alveolar bone, substantia ossea dentis, male pattern alopecia or alopecia senilis by preventing epilation or providing hair germination and/or promoting hair generation and hair growth, muscular dystrophy, pyoderma and Sezary's syndrome, ischemia-reperfusion injury of organs which occurs upon preservation, transplantation or ischemic disease, endotoxin-shock, pseudomembranous colitis, colitis caused by drug or radiation, ischemic acute renal insufficiency, chronic renal insufficiency, toxinosis caused by lung-oxygen or drugs, lung cancer, pulmonary emphysema, siderosis, retinitis pigmentosa, senile macular degeneration, vitreal scarring, corneal alkali burn, linear IgA ballous dermatitis and cement dermatitis, gingivitis, sepsis, diseases caused by environmental pollution, aging, carcinogenesis, metastasis of carcinoma and hypobaropathy, disease caused by histamine or leukotriene-C4 release, autoimmune hepatitis, sclerosing cholangitis, partial liver resection, acute liver necrosis, necrosis caused by toxin, viral hepatitis, shock, or anoxia, B-virus hepatitis, non-A/non-B hepatitis, cirrhosis, alcoholic cirrhosis, hepatic failure, fulminant hepatic failure, late-onset hepatic failure, “acute-on-chronic” liver failure, augmentation of chemotherapeutic effect, cytomegalovirus infection, HCMV infection, AIDS, senile dementia, Parkinson's diseases, trauma, and chronic bacterial infection.

In certain embodiments, inflammatory diseases and conditions that may be treated or prevented by administering a compound or composition of the invention include Lupus nephritis, Type II diabetes, Hyperimmunoglobulinemia D and periodic fever syndrome, Schnitzler's syndrome, Adult's onset Still's disease, Pseudogout, SAPHO syndrome, Castleman's disease and Alzheimer's disease.

Further conditions that can benefit from administration of a compound or composition of the invention include Huntington's disease, Kidney fibrosis, Liver fibrosis, Non-alcoholic steatohepatitis (NASH) and neuroinflammation. It should be mentioned that many fibrosis is a pathological feature of most chronic inflammatory diseases and thus that fibrosis and inflammation frequently occur together.

Compounds and compositions of the invention also find utility in a method of treating or preventing a disease or disorder, said method comprising a step of administering a compound of formula (I), or a composition of the invention, to a subject in need thereof. As such, a compound or composition of the invention may be administered to a subject suffering, or at risk of developing, a cancer, an inflammatory disease, an autoinflammatory diseases and/or an autoimmune disease.

Compounds of formula (I) of the invention also find use in the manufacture of a medicament, particularly use in the manufacture of a medicament to be administered to a subject suffering, or at risk of developing, a cancer, an inflammatory disease, an autoinflammatory diseases and/or an autoimmune disease.

The amount of a compound of formula (I) which is required to achieve a therapeutic effect will vary with particular route of administration and the characteristics of the subject under treatment, for example the species, age, weight, sex, medical conditions, the particular disease or condition and its severity, and other relevant medical and physical factors. An ordinarily skilled physician can readily determine and administer an effective amount of the compound of formula (I) required for treatment or prophylaxis of a disease or condition.

The compound of formula (I) may be administered daily (including several times daily), every second or third day, weekly, every second, third or fourth week or even as a high single dose depending on the subject and disease or disorder to be treated.

Preferably, the compound of formula (I) (excluding the mass of any counterion or solvent), may be administered in an amount of about 1 to 1000 mg per administration. For example, 1, 5, 10, 15, 20, 25, 40, 50, 60, 70, 80, 90, 100, 110, 120, 130, 140, 150, 200, 300, 400, 500, 600, 700, 800, 900 and 1000 mg.

In certain embodiments, the compound of formula (I) is administered as a composition. Preferably, the composition is a pharmaceutical composition of the present invention.

Whilst a compound of formula (I) may be used as the sole active ingredient in the present invention, it is also possible for it to be used in combination with one or more further therapeutic agent(s), and the use of such combinations provides one embodiment of the invention. Such further therapeutic agents may be agents useful in the treatment or prophylaxis of a disease or condition, or other pharmaceutically active materials. Such agents are known in the art. Examples of further therapeutic agents for use in the present invention include those described herein.

The one or more further therapeutic agent(s) may be used simultaneously, sequentially or separately with/from the administration of the dosage a compound of formula (I). The individual components of such combinations can be administered separately at different times during the course of therapy or concurrently in divided or single combination forms. An ordinarily skilled physician can readily determine and administer the effective amount of one or more therapeutic agent required to have the desired therapeutic effect.

Preferred unit dosage compositions for use according to the invention are those containing an effective dose, or an appropriate fraction thereof, of the compound of formula (I).

### Kits

The present invention provides a kit comprising a compound of formula (I), one or more pharmaceutically acceptable excipients, and optionally one or more further therapeutic agents. Examples of such further therapeutic agents include those described herein as being suitable for use in the present invention, and being optionally present in a pharmaceutical composition of the invention as a further therapeutic agent.

Kits of the present invention find use in the treatment and/or prophylaxis of cancers, inflammatory diseases, autoinflammatory diseases and autoimmune diseases.

For the avoidance of doubt, the compound of formula (I) present in a kit according to the present invention is in a form and quantity suitable for use according to the present invention. Suitable pharmaceutical compositions and formulations are described herein. The skilled person can readily determine a quantity of the compound of formula (I) suitable for including in a kit of the invention, and for use according the invention.

### Exemplary Procedures for the Preparation of Compounds of Formula (I)

The compounds of the invention may be prepared using methods known to those skilled in the art of organic chemistry. Exemplary procedures for the preparation of compounds of formula (I) are provided below in Scheme 1 to 4. The invention provides a method of preparing a compound of the invention by a process as depicted in any one of Schemes 1 to 4. Analogous procedures can be used for all compounds of formula (I) in claim 1, including those with W₁, W₂ and W₃ groups rather than W, or alternative structures for the cyclic group ‘A’. For the avoidance of doubt, groups R¹, R², R³, W, X, Y and A of intermediate compounds (II) to (XVIII) described herein are the same as the R¹, R², R³, W, X, Y and A groups described herein for formula (I).

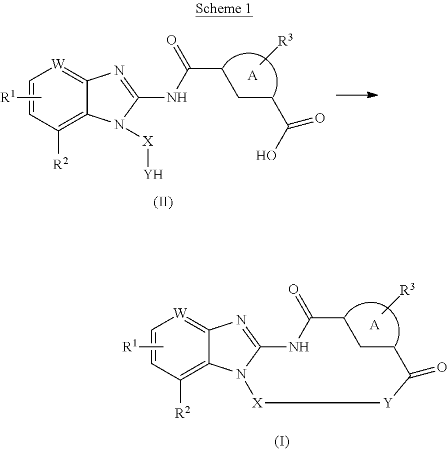

As shown in Scheme 1, compounds of formula (I) may obtained by cyclization of carboxylic acid intermediate (II). Cyclisation may be achieved using coupling reagents (such as HOBT, HATU) under dilute conditions. Preferably this step is performed using HATU (1.2 eq) and DIPEA (3 eq) in THF (final concentration of starting material is 0.5 mM or less) with heating at 70° C. for more than 2 hours. The reaction mixture is then cooled and filtered through silica gel.

An alternative approach towards compounds of formula (I) is depicted below in Scheme 2. Said approach involves the cyclisation of carboxylic acid intermediate (III) using a suitable amide coupling agent, such as HATU.

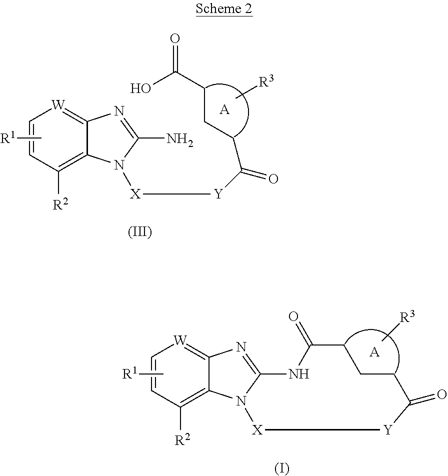

A further approach towards compounds of formula (I) is depicted in below in Scheme 3. Said approach involves a ring closing metathesis reaction of intermediate (IV), wherein Z¹ and Z² are independently selected from bond or C₁₋₃alkylene, using a suitable catalyst (e.g. Grubbs catalyst) to form intermediate (V). Intermediate (V) may then be hydrogenated to provide a compound of formula (I) wherein X in formula (I) shown below is —Z¹-ethylene-Z²—.

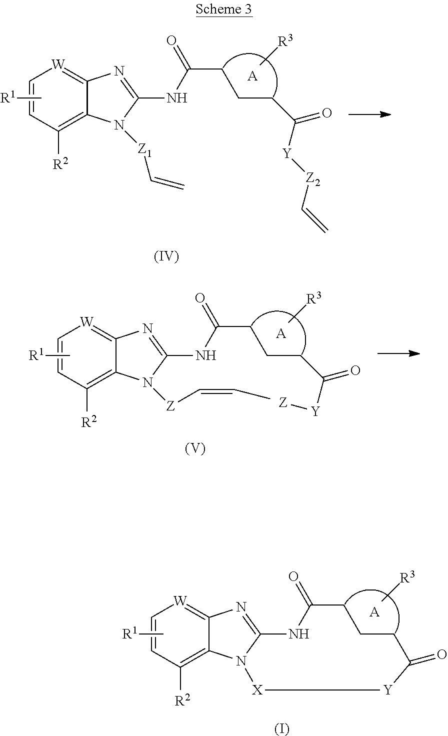

Further exemplary procedures for the preparation of compounds of formula (I) are depicted below in Scheme 4.

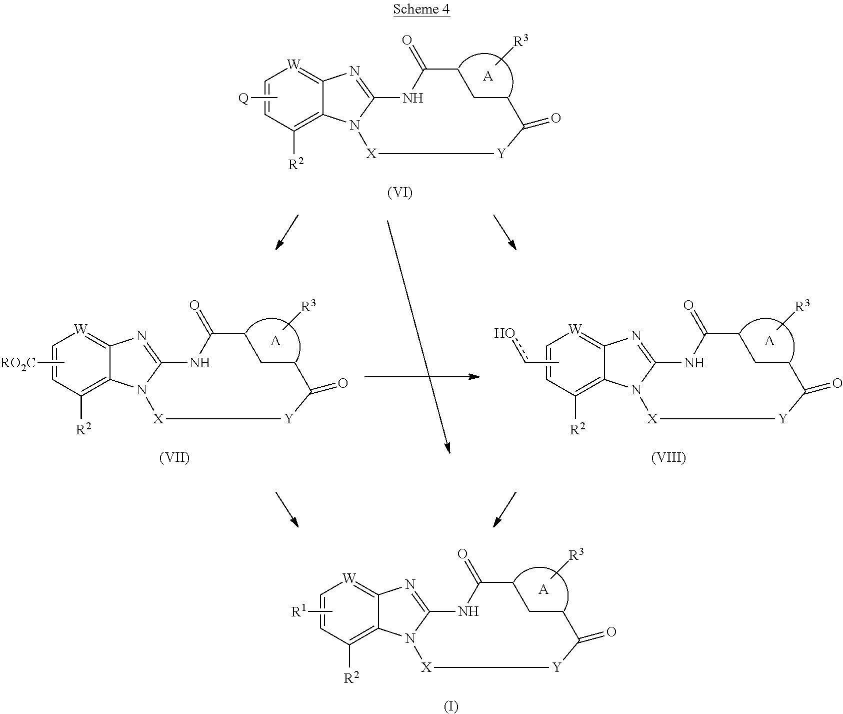

Intermediate (VI), wherein Q is a halogen, boronic acid, boronic ester, triflate, can be prepared in a similar fashion to the chemistry depicted above in Schemes 1 to 3. Intermediates (VII) and (VIII) or a compound of formula (I) can be prepared from intermediate (VI) through transition metal coupling reactions. For example, intermediate (VII) can be prepared through a Pd-catalyzed carbonylation of intermediate (VI). The compound of formula (I) can be prepared from intermediate (VII) or intermediate (VIII) through amide coupling reactions, reductive aminations or nucleophilic substitution reactions. The compound of formula (I) can also be prepared directly from intermediate (VI).

An exemplary procedure for the preparation of intermediate (II) is depicted below in Scheme 5.

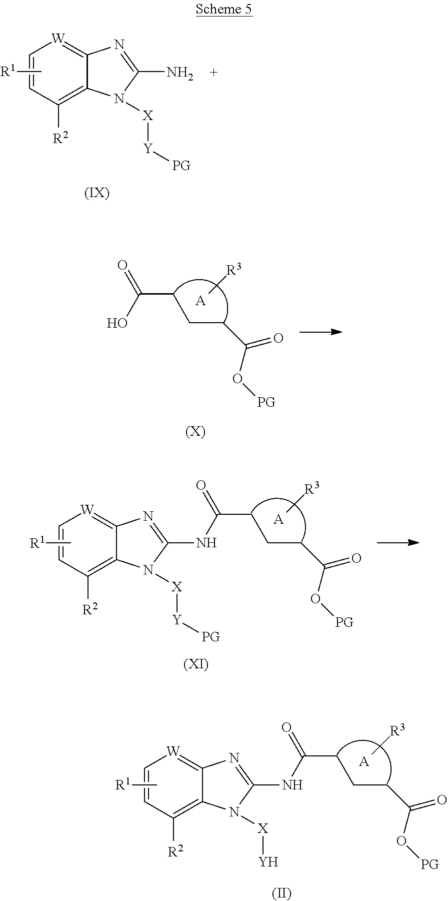

Intermediate (XI) is prepared by coupling of compounds corresponding to intermediate (IX) with a suitable mono-protected diacid, intermediate (X), using a suitable coupling reagent (e.g. HATU, HOBT). Finally, (XI) can be deprotected to provide intermediate (II).

For the avoidance of doubt, the group “PG” as used herein represents suitable a protecting group. Protecting groups may be added and subsequently removed from, the various reactants and intermediates in a manner that will be readily understood by one skilled in the art of organic synthesis. Conventional procedures for using such protecting groups, as well as examples of suitable protecting groups, are known in the art. Further, such procedures and groups are described in the literature, such as in “Protective Groups in Organic Synthesis”, 3rd ed., T. W. Green, P. G. M. Wuts, Wiley-Interscience, New York (1999).

An exemplary procedure for the preparation of intermediates of formula (III) is depicted below in Scheme 6.

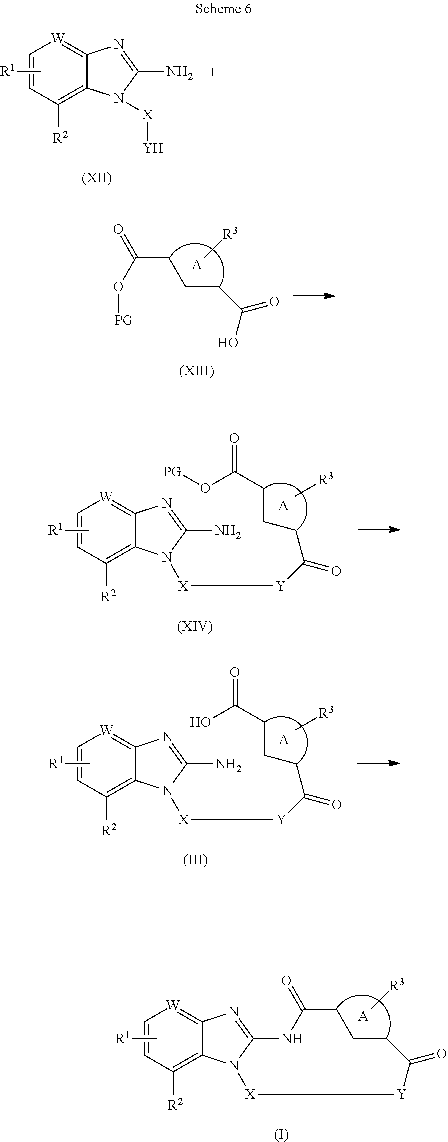

Intermediate (XI) can be prepared through coupling of compounds corresponding to intermediate (XII) with a suitable mono-protected diacid, intermediate (XIII), using a suitable coupling reagent (e.g. HATU, HOBT). Finally, intermediate (XIV) can be deprotected to provide intermediate (III).

An exemplary procedure for the preparation of intermediate (IX) is depicted below in Scheme 7.

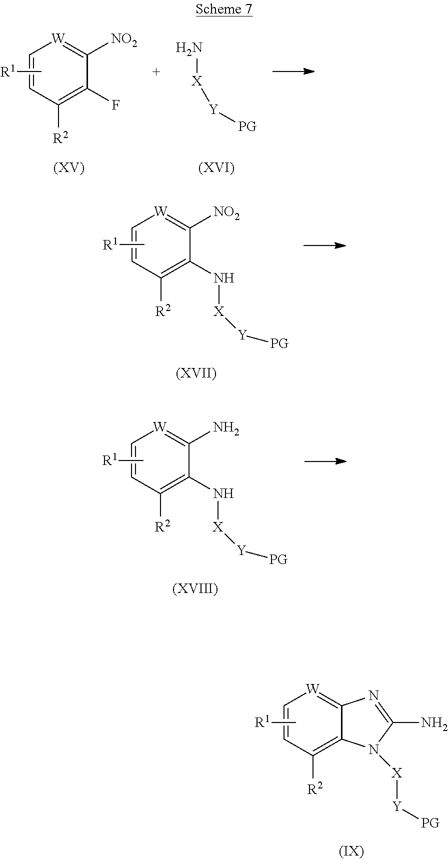

Amines corresponding to intermediate (XVI) can be reacted with fluoro-nitro derivatives corresponding to intermediate (XV) through a nuclear aromatic substitution reaction in the presence of a suitable base (e.g. TEA, KOtBu) at room temperature or with heating. The resulting nitro-aniline, intermediate (XVII), can be reduced (e.g. using hydrogen and a suitable catalyst, Zn in AcOH, SnCl₂, Fe in HCl) to give intermediate (XVIII). Treating intermediate (XVIII) with cyanogen bromide (CNBr) induces a cyclization to provide intermediate (IX).

### EQUIVALENTS

The invention has been described broadly and generically herein. Those of ordinary skill in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention. Further, each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

### INCORPORATION BY REFERENCE

The contents of the articles, patents, and patent applications, and all other documents and electronically available information mentioned or cited herein, are hereby incorporated by reference in their entirety to the same extent as if each individual publication was specifically and individually indicated to be incorporated by reference. Applicants reserve the right physically to incorporate into this application any and all materials and information from any such articles, patents, patent applications, or other physical and electronic documents.

The following Examples illustrate the invention.

### EXAMPLES

Reagents and solvents were purchased from commercial suppliers and used as received. Reactions were stirred using Teflon-coated magnetic stir bars in glass vials or round bottomed flasks and heated using conventional stirring plates.

Solvents were removed on rotary evaporators, on a Genevac EZ-2 vacuum centrifuge, or by freeze-drying.

Reaction progress was monitored by LC-MS or thin layer chromatography (TLC) using aluminium-backed plates (Merck 60F254). TLC plates were visualized by UV-light (254 nm). LC-MS were acquired on either an Agilent 1100 system coupled with an Agilent MSD mass spectrometer operating in ES (+) ionization mode, using a Purosphere STAR RP-18, 2.1×50 mm, endcapped 3 μm, column and eluted with solution A (water with 0.1% TFA) and B (acetonitrile), or an Agilent 1260 Infinity II system coupled with an Agilent MSD XT mass spectrometer operating in ES (+ or −) ionization mode, using a Gemini NX-C18, 3.0×50 mm, 110 Å, column and eluted with solution A (water with 0.2% NH₄OH) and B (acetonitrile). UV-traces were recorded at 220 or 254 nm. HPLC were performed on an Agilent 1100 system using a Kromasil Eternity-5-C 18, 4.6×150 mm column and eluted with solution A (water with 0.1% TFA) and B (acetonitrile). UV-traces were recorded at 220 or 254 nm.

Preparative HPLC were performed either on a Gilson system using a Kromasil 100-5C18, 21.1×250 mm, column and eluted with solution A (water with 0.1% TFA) and B (acetonitrile), or on a Agilent 1200 system using a Phenomenex Gemini NX-C18 110 Å, 21.2×150 mm, 5 μm column, and eluted with solution A (50 mM aq. NH₄OH) and B (acetonitrile).

Flash chromatography on silica gel or C18 functionalized silica were performed on a Biotage Isolera One system equipped with a diode array detector, using the solvent systems indicated. UV traces were recorded between 200 nm and 400 nm.

Nuclear Magnetic Resonance (NMR) spectra were recorded either on a 400 MHz (1H NMR at 400 MHz and ¹³C NMR at 101 MHz) Varian Inova spectrometer equipped with a 5 mm ¹H/¹³C auto-switchable gradient-probe at 25° C., or on a 500 MHz Bruker Avance Neo spectrometer equipped with a 5 mm iProbe BBF/H/D probe. Spectra were processed using MestReNova v. 12.0. Chemical shifts are reported in ppm (δ) using the residual solvent as internal standard. Peak multiplicities given in Hz are expressed as follow: s, singlet; d, doublet; dd, doublet of doublets; ddd, doublet of doublet of doublets; t, triplet; dt, doublet of triplets; q, quartet; dq, doublet of quartets; p, pentet; h, heptet; m, multiplet; br s, broad singlet.

Abbreviations used herein include:
DIPEA N,N-Diisopropylethylamine;
DMAP 4-(Dimethylamino)pyridine;
DMF N,N-dimethylformamide;
DMP Dess-Martin periodinane;
DMSO Dimethyl sulfoxide;
EDC N-Ethyl-N′-(3-dimethylaminopropyl)carbodiimide;
HATU 1-[Bis(dimethylamino)methylene]-1H-1,2,3-triazolo[4,5-b]pyridinium 3-oxid hexafluorophosphate;
HCl hydrochloric acid;
HOBT 1-Hydroxybenzotriazole;
IPA isopropyl alcohol;
on overnight;
pet. ether petroleum ether;
quant. quantitatively;
rt room temperature;
Rt Retention time;
sat. Saturated;
SFC Supercritical fluid chromatography;
SiO₂ silica gel;
TBAF Tetrabutylammonium fluoride;
tBuOK potassium tert-butoxide;
TCFH Chloro-N,N,N′,N′-tetramethylformamidinium hexafluorophosphate
TEA Triethyl amine;
TFA Trifluoroacetic acid;
THF tetrahydrofuran;
TIPSOTf Triisopropylsilyl trifluoromethanesulfonate;
TLC thin layer chromatography;
TMS tetramethylsilane.

The chemical structures of the Example compounds disclosed herein are shown as having the tautomeric form depicted in formula (I) or formula (Ie). Although a specific tautomeric form is shown hereinbelow, the example compounds may also be in the alternative tautomeric form to the one depicted herein. The compound names were obtained using the IUPAC naming module in ChemDraw 20.0.

### Example 1: 1¹H-2,6-diaza-1(2,1)-benzo[d]imidazola-4(1,3)-benzenacyclododecaphane-3,5-dione

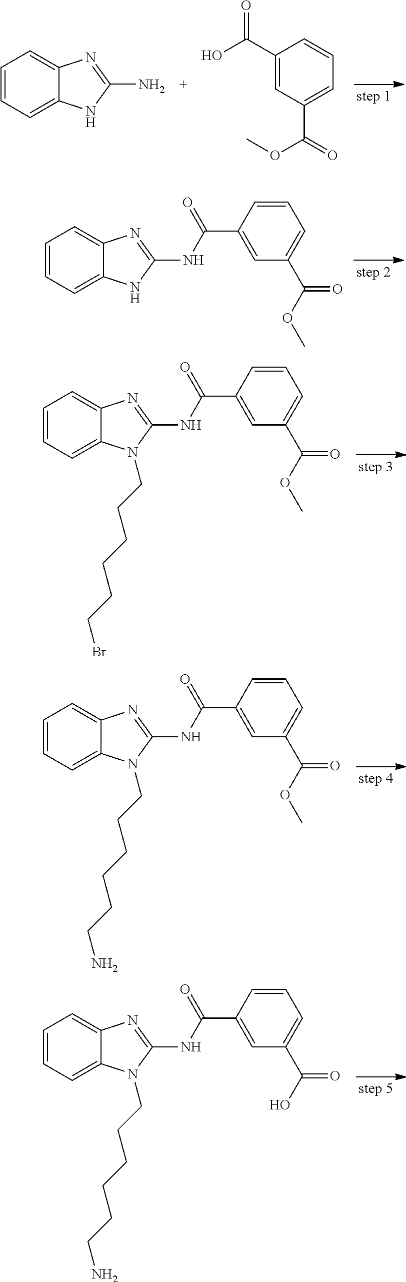

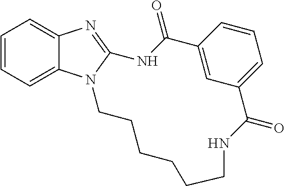 [SMILES: O=C1NCCCCCCn2c(nc3ccccc32)NC(=O)c2cccc1c2]

### Step 1: Preparation of methyl 3-((1H-benzo[d]imidazol-2-yl)carbamoyl)benzoate

To a stirred solution of aminobenzimidazole (1.07 g, 8.00 mmol) and 3-(methoxycarbonyl)benzoic acid (1.44 g, 8.0 mmol) in 10 ml CH₂Cl₂ was added HOBT monohydrate (1.23 g, 8.0 mmol), EDC (1.53 g, 8.00 mmol), DMAP (98 mg, 0.8 mmol) and DIPEA (2.58 g, 8.0 mmol). The reaction was stirred over night at room temperature and then concentrated to dryness under vacuum. The residue was dissolved in 45 ml EtOH. The product precipitated and was collected as a white solid by filtration to give 532 mg (21% yield). ¹H NMR (DMSO-d₆) δ: 12.47 (s, 1H), 8.78 (m, 1H), 8.39 (m, 1H), 8.12 (m, 1H), 7.66 (m, 1H), 7.45 (m, 2H), 7.17 (m, 2H), 3.91 (s, 3H); LC/MS: M+H=296.1.

### Step 2: Preparation of methyl 3-((1-(6-bromohexyl)-1H-benzo[d]imidazol-2-yl)carbamoyl)benzoate

To a stirred solution of methyl 3-((1H-benzo[d]imidazol-2-yl)carbamoyl)benzoate (348 mg, 1.18 mmol) and KOtBu (139 mg, 1.24 mmol) in 8 ml THF and 2 ml DMSO was added 1,6-dibromohexane at room temperature. The reaction was stirred over night and the diluted with 60 ml water. The mixture was extracted with three portions of EtOAc (20 ml). The organic phases were combined, dried and concentrated. The residue was subjected to flash chromatography to afford the desired of methyl 3-((1-(6-bromohexyl)-1H-benzo[d]imidazol-2-yl)carbamoyl)benzoate (374 mg, 69%) as an oil. LC/MS: M+H=460.1, Rₜ: 5.4 min.

### Step 3: Preparation of methyl 3-((1-(6-aminohexyl)-1H-benzo[d]imidazol-2-yl)carbamoyl)benzoate

Sodium azide (58 mg, 0.9 mmol) was added to a solution of 3-((1-(6-bromohexyl)-1H-benzo[d]imidazol-2-yl)carbamoyl)benzoate (374 mg, 0.82 mmol) in 2 ml DMF at room temperature. The reaction was stirred overnight and then diluted with 1.0 ml EtOH. Pd/C (87 mg, 0.08 mmol) was added and the reaction mixture was put under an atmosphere of H₂. The reaction was stirred overnight, filtered through celite with EtOH and the filtrate was concentrated. The residue was dissolved in MeCN and water (2 ml) and purified by reverse phase chromatography on a C18 flash cartridge (Biotage 12G Ultra C18 column) using 5-80% MeCN in water with 1% TFA. The fractions were collected and freeze dried to give a pink solid (229 mg). The material was used in the next step without further purification. LC/MS: M+H=395.2, Rₜ: 3.9 min.

### Step 4: Preparation of 3-((1-(6-aminohexyl)-1H-benzo[d]imidazol-2-yl)carbamoyl)benzoic acid

To a stirred solution of methyl 3-((1-(6-aminohexyl)-1H-benzo[d]imidazol-2-yl)carbamoyl)benzoate (217 mg, 0.55 mmol) in 3 ml MeOH was added KOH (93 mg, 1.65 mmol). After 4 h at room temperature the reaction was heated at 65° C. for 1.5 h. The reaction was diluted with 1 ml water and cooled to room temperature. The reaction mixture was concentrated using a nitrogen flow over night. The residue was dissolved in 1 M HCl and MeCN to a volume of 1 ml and purified by reverse phase chromatography on a C18 flash cartridge (Biotage 12G Ultra C18 column) using 5-80% MeCN in water with 1% TFA. The fractions were collected and freeze dried to give a white solid (96 mg, 35% yield). LC/MS: M+H=381.1, Rₜ: 3.7 min.

### Step 5: Preparation of 1¹H-2,6-diaza-1(2,1)-benzo[d]imidazola-4(1,3)-benzenacyclododecaphane-3,5-dione

To a solution of 3-((1-(6-aminohexyl)-1H-benzo[d]imidazol-2-yl)carbamoyl)benzoic acid (45 mg, 0.09 mmol) and HOBT monohydrate (15 mg, 0.1 mmol), EDC (19 mg, 0.1 mmol), DMAP (1.1 mg, 0.009 mmol) was added DIPEA (63 μl, 0.36 mmol). The reaction mixture was stirred over night at room temperature. The reaction mixture was filtered and the filtrate was concentrated. The residue was dissolved in water and MeCN to a volume of 1 ml and purified by reverse phase chromatography on a C18 flash cartridge (Biotage 12G Ultra C18 column) using 5-95% MeCN in water with 1% TFA. The fractions were collected and freeze dried to give a white solid (4.8 mg, 11% yield, 80% purity by HPLC). ¹H NMR (DMSO-d₆) δ: 12.47 (bs, 1H), 9.22 (s, 1H), 8.30 (s, 1H), 8.05 (m, 1H), 7.69 (d, 1H), 7.53 (m, 2H), 7.48 (m, 1H), 7.28-7.17 (m, 2H), 4.23 (bs, 2H), 3.28 (bs, 2H), 1.86 (bs, 2H), 1.72 (bs, 2H), 1.66-1.51 (m, 4H); LC/MS: M+H=363.1.

### Example 2: 1¹H-2,6-diaza-1(2,1)-benzo[d]imidazola-4(1,3)-benzenacycloundecaphane-3,5-dione

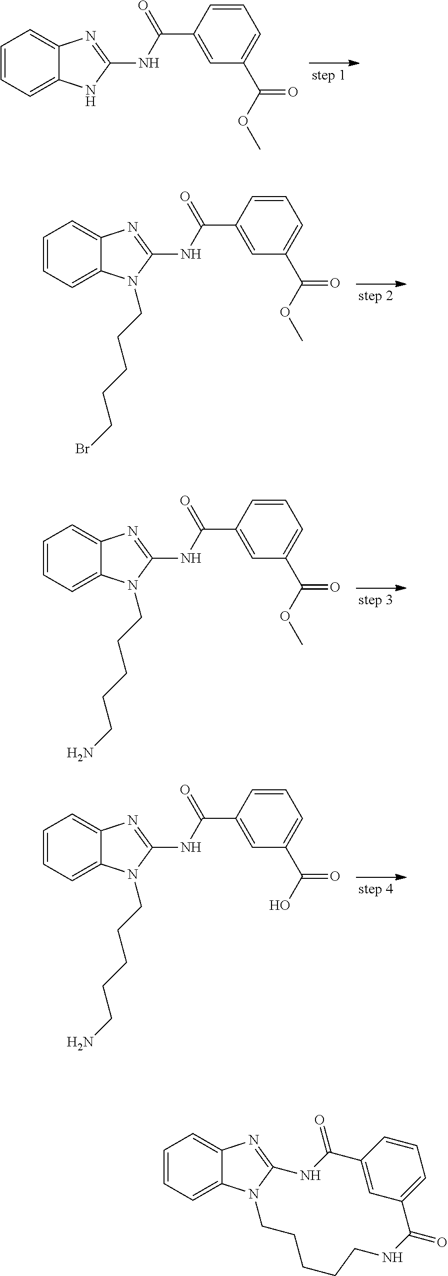 [SMILES: COC(=O)c1cccc(C(=O)Nc2nc3ccccc3[nH]2)c1 ; COC(=O)c1cccc(C(=O)Nc2nc3ccccc3n2CCCCCBr)c1 ; COC(=O)c1cccc(C(=O)Nc2nc3ccccc3n2CCCCCN)c1 ; NCCCCCn1c(NC(=O)c2cccc(C(=O)O)c2)nc2ccccc21 ; O=C1NCCCCCn2c(nc3ccccc32)NC(=O)c2cccc1c2]

### Step 1: Preparation of methyl 3-((1-(5-bromopentyl)-1H-benzo[d]imidazol-2-yl)carbamoyl)benzoate

To a stirred solution of methyl 3-((1H-benzo[d]imidazol-2-yl)carbamoyl)benzoate (1.68 g, 5.57 mmol) and KOtBu (688 mg, 6.13 mmol) in 20 ml DMF was added 1,5-dibromopentane at room temperature. The reaction was stirred overnight and the diluted with 200 ml water. The mixture was extracted with three portions of EtOAc/petroleum ether. The aqueous phase was extracted with CH₂Cl₂. The organic phases were combined, dried and concentrated. The residue was subjected to flash chromatography to afford the desired of methyl 3-((1-(6-bromohexyl)-1H-benzo[d]imidazol-2-yl)carbamoyl)benzoate (1.45 g, 58%) as an oil. ¹H NMR (DMSO-d₆) δ: 12.78 (s, 1H), 8.82 (s, 1H), 8.49 (d, 1H), 8.09 (d, 1H), 7.95 (s, 1H), 7.63 (m, 12H), 7.55 (m, 2H), 7.25 (m, 2H), 4.30 (m, 2H), 3.91 (s, 3H), 3.50 (m, 2H), 1.86 (m, 4H), 1.49 (m, 2H); LC/MS: M+H=458.1.

### Step 2: Preparation of methyl 3-((1-(5-aminopentyl)-1H-benzo[d]imidazol-2-yl)carbamoyl)benzoate

To a stirred solution of methyl 3-((1-(5-bromopentyl)-1H-benzo[d]imidazol-2-yl)carbamoyl)benzoate (889 mg, 2 mmol) in 5 ml DMF was added sodium azide (143 mg, 2.20 mmol) at room temperature. The reaction was stirred overnight and then diluted with 2.5 ml EtOH. Pd/C (218 mg, 0.2 mmol) was added and the reaction mixture was put under an atmosphere of H₂. The reaction was stirred overnight, filtered through celite with EtOH and the filtrate was concentrated. The residue was dissolved in MeCN and water (2 ml) and purified by reverse phase chromatography on a C18 flash cartridge (Biotage 12G Ultra C18 column) using 0-70% MeCN in water with 1% TFA. The fractions were collected and freeze dried to give a white solid (439 mg, 44% yield as TFA salt). ¹H NMR (DMSO-d₆) δ: 8.81 (s, 1H), 8.49 (d, 1H), 8.10 (d, 1H), 7.60 (m, 3H), 7.26 (m, 2H), 4.29 (m, 2H), 3.91 (s, 3H), 2.76 (s, 2H), 1.86 (s, 2H), 1.61 (d, 2H), 1.41 (s, 2H); LC/MS: M+H=381.2.

### Step 3: Preparation of 3-((1-(5-aminopentyl)-1H-benzo[d]imidazol-2-yl)carbamoyl)benzoic acid

To a stirred solution of methyl 3-((1-(5-aminopentyl)-1H-benzo[d]imidazol-2-yl)carbamoyl)benzoate (342 mg, 0.9 mmol) in 5 ml THF was added an aqueous solution of LiOH (67 mg, 2.79 mmol in 1 ml water) at room temperature. The reaction mixture was heated at 45° C. over night, cooled to room temperature and concentrated to dryness under vacuum. The residue was dissolved in 1 M HCl and MeCN to a volume of 1 ml and purified by reverse phase chromatography on a C18 flash cartridge (Biotage 12G Ultra C18 column) using 5-80% MeCN in water with 1% TFA. The fractions were collected and freeze dried to give a white solid (620 mg) that was used without further purification. LC/MS: M+H=367.1; Rₜ: 3.2 min.

### Step 4: Preparation of 1¹H-2,6-diaza-1(2,1)-benzo[d]imidazola-4(1,3)-benzenacycloundecaphane-3,5-dione

DIPEA (249 μl, 1.43 mmol) was added to a solution of 3-((1-(5-aminopentyl)-1H-benzo[d]imidazol-2-yl)carbamoyl)benzoic acid (150 mg, 0.41 mmol) and HATU (171 mg, 0.45 mmol) in 8 ml DMF at room temperature. The reaction was stirred over night. The reaction mixture was purified by reverse phase chromatography on a C18 flash cartridge (Biotage 12G Ultra C18 column) using 0-80% MeCN in water with 1% TFA. The fractions were collected and freeze dried. The product was further purified by precipitation from EtOH to give a white solid (2.5 mg, 2% yield). ¹H NMR (DMSO-d₆) δ: 12.51 (s, 1H), 9.04 (s, 1H), 8.11 (d, 1H), 7.88 (m, 1H), 7.74 (d, 1H), 7.58 (m, 1H), 7.53-7.48 (m, 2H), 7.23 (m, 2H), 4.27 (m, 2H), 3.19 (m, 2H), 2.10-1.99 (m, 2H), 1.85-1.79 (m, 2H), 1.48-1.37 (m, 2H); LC/MS: M+H=349.1.

### Example 3: 1⁵-((4-methylpiperazin-1-yl)methyl)-1¹H-2,6-diaza-1(2,1)-benzo[d]imidazola-4(1,3)-benzenacycloundecaphane-3,5-dione

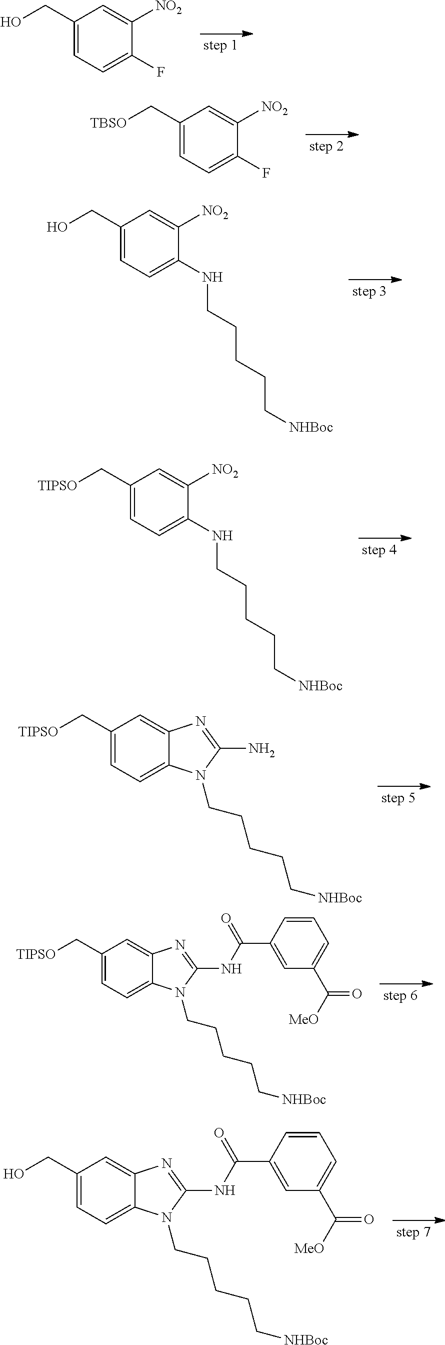

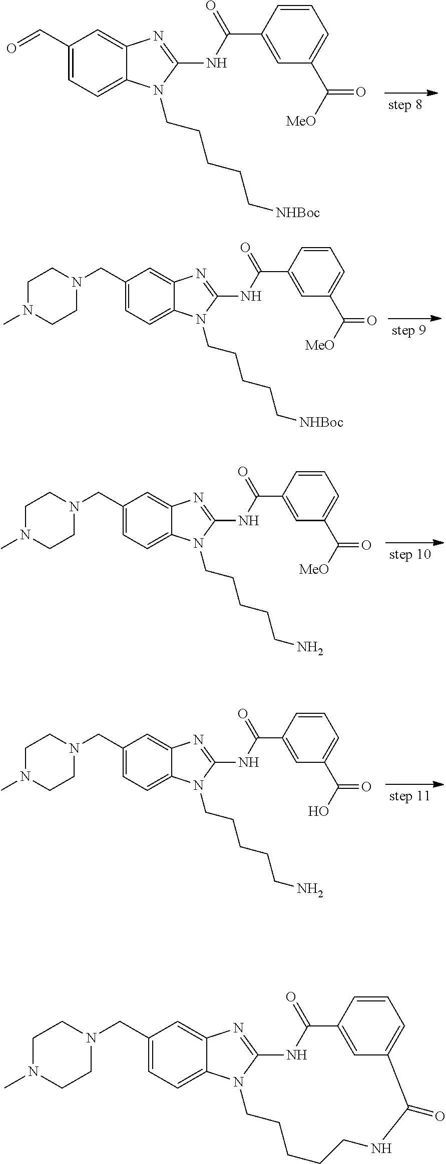 [SMILES: COC(=O)c1cccc(C(=O)Nc2nc3cc(C=O)ccc3n2CCCCCNC(=O)OC(C)(C)C)c1 ; COC(=O)c1cccc(C(=O)Nc2nc3cc(CN4CCN(C)CC4)ccc3n2CCCCCNC(=O)OC(C)(C)C)c1 ; COC(=O)c1cccc(C(=O)Nc2nc3cc(CN4CCN(C)CC4)ccc3n2CCCCCN)c1 ; CN1CCN(Cc2ccc3c(c2)nc(NC(=O)c2cccc(C(=O)O)c2)n3CCCCCN)CC1 ; CN1CCN(Cc2ccc3c(c2)nc2n3CCCCCNC(=O)c3cccc(c3)C(=O)N2)CC1]

### Step 1: Preparation of ((4-fluoro-3-nitrobenzyl)oxy)triisopropylsilane

TIPSOTf (2.02 ml, 7.50 mmol) was added slowly to a solution of (4-fluoro-3-nitrophenyl)methanol (856 mg, 5.0 mmol) and 2,6-lutidine (1.74 ml, 15 mmol) in 5 ml CH₂Cl₂. The reaction was stirred at room temperature for 4 h and then concentrated under vacuum. The residue was dissolved in CH₂Cl₂ and purified using flash chromatography (SiO₂, 0-25% EtOAc in petroleum ether) to give the product as a yellow oil (1.27 g, 77% yield). ¹H NMR (DMSO-d₆) δ: 8.12 (d, 1H), 7.83-7.70 (m, 1H), 7.59 (m, 1H), 4.88 (s, 2H), 1.17 (m, 3H), 1.09-1.02 (m, 18H).

### Step 2: Preparation of tert-butyl (5-((4-(hydroxymethyl)-2-nitrophenyl)amino)pentyl)carbamate

To a stirred solution of tert-butyl (5-aminopentyl)carbamate (445 mg, 2.20 ml) in 4 ml DMF was added ((4-fluoro-3-nitrobenzyl)oxy)triisopropylsilane (600 mg, 1.83 mmol) followed by DIPEA (1.55 ml, 3.66 mmol) at room temperature. The mixture was heated to 80° C. and stirred for 4 hours. The reaction mixture was cooled to room temperature and concentrated to dryness under vacuum. The residue was purified using flash chromatography (SiO₂, 10-60% EtOAc in petroleum ether) to give the product as a red oil (605 mg, 93% yield). ¹H NMR (DMSO-d₆) δ: 8.11 (m, 1H), 8.01 (s, 1H), 7.49 (m, 1H), 7.05 (d, 1H), 6.79 (m, 1H), 5.20 (m, 1H), 4.40 (d, 2H), 3.41-3.33 (m, 2H), 2.92 (m, 2H), 1.62 (m, 2H), 1.50-1.29 (m, 13H)

### Step 3: Preparation of tert-butyl (5-((2-nitro-4-(((triisopropylsilyl)oxy)methyl)phenyl)amino)pentyl)carbamate

TIPSOTf (0.35 μl, 1.28 mmol) was added slowly to a solution of tert-butyl (5-((4-(hydroxymethyl)-2-nitrophenyl)amino)pentyl)carbamate (302 mg, 0.86 mmol) and 2,6-lutidine (0.3 ml, 2.56 mmol) in 3 ml CH₂Cl₂. The reaction was stirred at room temperature over night and then concentrated under vacuum. The residue was dissolved in CH₂Cl₂ and purified using flash chromatography (SiO₂, 0-25% EtOAc in petroleum ether) to give the product as a yellow oil (326 mg, 75% yield). LC/MS: M+Na=532.3, Rₜ: 7.0 min.

### Step 4: Preparation of tert-butyl (5-(2-amino-5-(((triisopropylsilyl)oxy)methyl)-1H-benzo[d]imidazol-1-yl)pentyl)carbamate

To a stirred solution of tert-butyl (5-((2-nitro-4-(((triisopropylsilyl)oxy)methyl)phenyl)amino)pentyl)carbamate (326 mg, 0.64 mmol) in 35 ml MeOH under a nitrogen atmosphere was added Pd/C (68 mg, 0.064 mmol). The nitrogen was replaced by hydrogen and the reaction was stirred at room temperature for 2 hours. The reaction mixture was filtered through celite and washed with MeOH. The filtrate was concentrated under vacuum. The residue was dissolved in 10 ml water/MeOH/MeCN (1:3:1) and CNBr (81 mg, 0.77 mmol) was added at room temperature. The resulting solution was heated at 50° C. in a sealed flask overnight. The reaction mixture was diluted with 50 ml Na₂CO₃ (saturated aqueous solution) and extracted with EtOAc (3×30 ml). The organic phases were combined, dried and concentrated. The residue was dissolved in CH₂Cl₂ and purified using flash chromatography (SiO₂, 1-7% MeOH in CH₂Cl₂) to give the product as an oily film (137 mg, 42% yield). LC/MS: M+H=505.4, Rₜ: 5.6 min.

### Step 5: Preparation of methyl 3-((1-(5-((tert-butoxycarbonyl)amino)pentyl)-5-(((triisopropylsilyl)oxy)methyl)-1H-benzo[d]imidazol-2-yl)carbamoyl)benzoate

HATU (114 mg, 0.3 mmol) was added to a stirred solution of 3-(methoxycarbonyl)benzoic acid (59 mg, 0.33 mmol) in 5 ml DMF. The resulting solution was added to a stirred solution of tert-butyl (5-(2-amino-5-(((triisopropylsilyl)oxy)methyl)-1H-benzo[d]imidazol-1-yl)pentyl)carbamate (137 mg, 0.27 mmol) in 5 ml DMF followed by DIPEA (141 μl, 0.82 mmol). The reaction mixture was stirred over night at room temperature. The reaction mixture was concentrated to dryness under vacuum. The residue was dissolved in CH₂Cl₂ and purified using flash chromatography (SiO₂, 0-30% EtOAc in petroleum ether) to give the product as an oily film (160 mg, 88% yield). LC/MS: M+H=667.4, Rₜ: 6.6 min.

### Step 6: Preparation of methyl 3-((1-(5-((tert-butoxycarbonyl)amino)pentyl)-5-(hydroxymethyl)-1H-benzo[d]imidazol-2-yl)carbamoyl)benzoate

A solution of TBAF (0.66 ml 1M in THF, 0.66 mmol) was added to a stirred solution of methyl 3-((1-(5-((tert-butoxycarbonyl)amino)pentyl)-5-(((triisopropylsilyl)oxy)methyl)-1H-benzo[d]imidazol-2-yl)carbamoyl)benzoate (88 mg, 0.13 mmol) in 6 ml THF at room temperature. The reaction was stirred for 40 minutes and concentrated to dryness under vacuum. The residue was dissolved in CH₂Cl₂ and purified using flash chromatography (SiO₂, 1-6% MeOH in CH₂Cl₂ with 1% TEA) to give the product as a foam (56 mg, 83% yield). ¹H NMR (DMSO-d₆) δ: 12.75 (s, 1H), 8.82 (m, 1H), 8.48 (d, J=7.7 Hz, 1H), 8.09 (m, 1H), 7.63 (m, 1H), 7.54-7.52 (m, 1H), 7.47 (d, 1H), 7.21 (m, 1H), 6.71 (m, 1H), 5.23 (m, 1H), 4.56 (m, 2H), 4.26 (m, 2H), 3.91 (s, 3H), 2.89 (m, 2H), 1.82 (m, 2H), 1.47 (m, 2H), 1.39-1.22 (m, 9H), LC/MS: M+H=511.2.

### Step 7: Preparation of methyl 3-((1-(5-((tert-butoxycarbonyl)amino)pentyl)-5-formyl-1H-benzo[d]imidazol-2-yl)carbamoyl)benzoate

DMP (47 mg, 0.11 mmol) was added to a stirred solution of methyl 3-((1-(5-((tert-butoxycarbonyl)amino)pentyl)-5-(hydroxymethyl)-1H-benzo[d]imidazol-2-yl)carbamoyl)benzoate (47 mg, 0.09 mmol) in 5 ml CH₂Cl₂. The reaction was stirred at room temperature for 30 min, diluted with 20 ml CH₂Cl₂, washed with 1 M NaOH (aq) (3×10 ml), dried and concentrated to an oil. The crude product was used without further purification.

### Step 8: Preparation of methyl 3-((1-(5-((tert-butoxycarbonyl)amino)pentyl)-5-((4-methylpiperazin-1-yl)methyl)-1H-benzo[d]imidazol-2-yl)carbamoyl)benzoate

N-methyl piperazine (27 mg, 0.27 mmol) was added to a slurry of methyl 3-((1-(5-((tert-butoxycarbonyl)amino)pentyl)-5-formyl-1H-benzo[d]imidazol-2-yl)carbamoyl)benzoate (46 mg, 0.09 mmol) in 5 ml MeOH. The reaction mixture was diluted with 5 ml CH₂Cl₂ to give a clear solution. Sodium triacetoxyborohydride (58 mg, 0.027 mmol) was added. The mixture was stirred at room temperature for 5 days. The reaction mixture was concentrated under vacuum, diluted with 30 ml EtOAc, washed with Na₂CO₃ (saturated aqeuous solution, 3×20 ml). The organic phase was dried and concentrated to give an oil (58 mg). The crude product was used without further purification.

### Step 9: Preparation of methyl 3-((1-(5-aminopentyl)-5-((4-methylpiperazin-1-yl)methyl)-1H-benzo[d]imidazol-2-yl)carbamoyl)benzoate

TFA (1.0 ml, 8.61 mmol) was added to a solution of methyl 3-((1-(5-((tert-butoxycarbonyl)amino)pentyl)-5-((4-methylpiperazin-1-yl)methyl)-1H-benzo[d]imidazol-2-yl)carbamoyl)benzoate (54 mg, 0.09 mmol) in 10 ml CH₂Cl₂. The reaction was stirred at room temperature for 45 minutes and then concentrated to give an oil. The crude product was used without further purification.

### Step 10: Preparation of 3-((1-(5-aminopentyl)-5-((4-methylpiperazin-1-yl)methyl)-1H-benzo[d]imidazol-2-yl)carbamoyl)benzoic acid

KOH (31 mg, 0.55 mmol) was added to a stirred solution of methyl 3-((1-(5-aminopentyl)-5-((4-methylpiperazin-1-yl)methyl)-1H-benzo[d]imidazol-2-yl)carbamoyl)benzoate (86 mg, 0.09 mmol) in 10 ml MeOH at room temperature. The reaction was stirred at room temperature for 1 hour before heating to 60° C. After an additional 3 hours, 5 ml water was added and heating continued over night. The reaction was cooled to room temperature and concentrated to dryness under vacuum.

The residue was dissolved in MeCN and water (1 ml) and purified by reverse phase chromatography on a C18 flash cartridge (Biotage 12G Ultra C18 column) using 5-50% MeCN in water with 0.1% TFA. The fractions were collected and freeze dried to give a white solid (36 mg, 49% yield as TFA salt). LC/MS: M+H=479.3, Rₜ: 3.2 min.

### Step 11: Preparation of 1⁵-((4-methylpiperazin-1-yl)methyl)-1¹H-2,6-diaza-1(2,1)-benzo[d]imidazola-4(1,3)-benzenacycloundecaphane-3,5-dione

To a solution of 3-((1-(5-aminopentyl)-5-((4-methylpiperazin-1-yl)methyl)-1H-benzo[d]imidazol-2-yl)carbamoyl)benzoic acid (20 mg, 0.024 mmol) and DIPEA (21 μl, 0.12 mmol) in 10 ml DMF, was added HATU (11 mg, 0.029 mmol). After 70 minutes the reaction mixture was concentrated to dryness under vacuum. The residue was dissolved in MeCN and water (1 ml) and purified by reverse phase chromatography on a C18 flash cartridge (Biotage 12G Ultra C18 column) using 5-40% MeCN in water with 0.1% TFA. The fractions were collected and freeze dried to give a grey solid (2.2 mg, 13% yield as TFA salt). ¹H NMR (DMSO-d₆) δ: 12.54 (s, 1H), 9.03 (s, 1H), 8.10 (d, 1H), 7.89 (m, 1H), 7.75 (d, 1H), 7.58 (m, 1H), 7.51 (d, 1H), 7.46 (s, 1H), 7.24 (d, 1H), 4.28 (s, 2H), 3.81 (s, 2H), 3.68-3.25 (m, 4H), 3.20 (s, 2H), 3.02 (s, 4H), 2.77 (s, 3H), 2.04 (s, 2H), 1.82 (s, 2H), 1.43 (s, 2H). LC/MS: M+H=461.2

### Example 4: 1⁵-methyl-1¹H-2,6-diaza-1(2,1)-benzo[d]imidazola-4(1,3)-benzenacycloundecaphane-3,5-dione

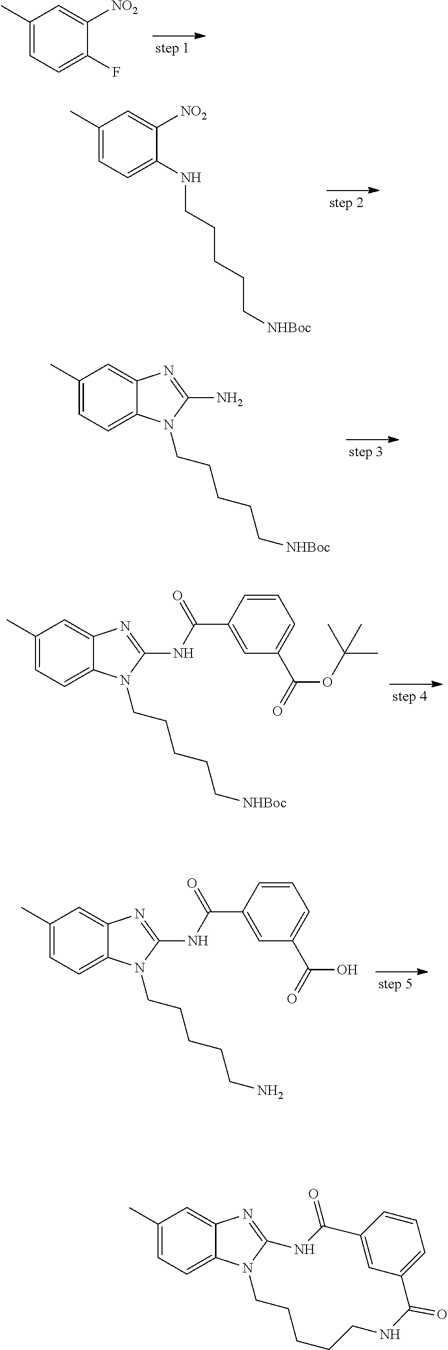

### Step 1: Preparation of tert-butyl (5-((4-methyl-2-nitrophenyl)amino)pentyl)carbamate

1-fluoro-4-methyl-2-nitrobenzene (776 mg, 5.0 mmol) and DIPEA (4.24 ml, 10.0 mmol) were added to a solution of tert-butyl (5-aminopentyl)carbamate (1.01 g, 5.0 mmol) in 10 ml DMF at room temperature. The reaction mixture was heated to 80° C. and stirred for 2.5 hours. The reaction mixture was cooled to room temperature, diluted with 150 ml water and extracted with EtOAc (3×50 ml). The combined organic phases were dried and concentrated. The residue was dissolved in CH₂Cl₂ and purified using flash chromatography (SiO₂, 10-50% EtOAc in petroleum ether) to give the product as a red oil (1.18 g, 70% yield). LC/MS: M+Na=360.1, Rₜ: 5.4 min.

### Step 2: Preparation of tert-butyl (5-(2-amino-5-methyl-1H-benzo[d]imidazol-1-yl)pentyl)carbamate

To a stirred solution of tert-butyl (5-((4-methyl-2-nitrophenyl)amino)pentyl)carbamate (1.18 g, 3.51 mmol) in 35 ml MeOH under a nitrogen atmosphere was added Pd/C (373 mg, 0.35 mmol). The nitrogen was replaced by hydrogen and the reaction was stirred at room temperature for 1.5 hours. The reaction mixture was filtered through celite and washed with MeOH. The filtrate was concentrated under vacuum. The residue was dissolved in 10 ml water/MeOH/MeCN (1:3:1) and CNBr (446 mg, 4.21 mmol) was added at room temperature. The resulting solution was heated at 50° C. in a sealed flask overnight. The reaction mixture was diluted with 50 ml Na₂CO₃ (saturated aqueous solution) and extracted with EtOAc (3×30 ml). The organic phases were combined, dried and concentrated. The residue was dissolved in CH₂Cl₂ and purified using flash chromatography (SiO₂, 1-10% MeOH in CH₂Cl₂ with 1% NH₃ (28% aqueous)) to give the product as a red brown solid (855 mg, 73% yield). LC/MS: M+H=333.2, Rₜ: 3.2 min.

### Step 3: Preparation of tert-butyl 3-((1-(5-((tert-butoxycarbonyl)amino)pentyl)-5-methyl-1H-benzo[d]imidazol-2-yl)carbamoyl)benzoate

tert-butyl (5-(2-amino-5-methyl-1H-benzo[d]imidazol-1-yl)pentyl)carbamate (33 mg, 0.1 mmol), 3-(tert-butoxycarbonyl)benzoic acid (22 mg, 0.1 mmol), HATU (38 mg, 0.1 mmol) and TEA (28 μl, 0.2 mmol) were dissolved in 0.4 ml MeCN and stirred at 50° C. over night. The reaction mixture was cooled to room temperature and diluted with water and MeOH and purified by reverse phase chromatography (C18, 50 mM NH₄OH (aq.)/MeCN gradient) to give the product (34 mg, 63% yield). ¹H NMR (DMSO-d₆) δ: 12.69 (s, 1H), 8.78 (s, 1H), 8.43 (d, 1H), 8.02 (d, 1H), 7.59 (m, 1H), 7.41 (d, 1H), 7.34 (s, 1H), 7.08 (d, 1H), 6.68 (m, 1H), 4.23 (m, 2H), 2.87 (m, 2H), 2.39 (s, 3H), 1.81 (m, 2H), 1.58 (s, 9H), 1.45 (m, 2H), 1.37-1.26 (m, 9H). LC/MS: M+H=537.3

### Step 4: Preparation of 3-((1-(5-aminopentyl)-5-methyl-1H-benzo[d]imidazol-2-yl)carbamoyl)benzoic acid

To a stirred solution of tert-butyl 3-((1-(5-((tert-butoxycarbonyl)amino)pentyl)-5-methyl-1H-benzo[d]imidazol-2-yl)carbamoyl)benzoate (34 mg, 0.063 mmol) in 2 ml dioxane and 1 ml water was added 1 ml HCl (conc.). The reaction was stirred over night and concentrated under vacuum. The residue was dissolved in toluene and concentrated to dryness under vacuum. The product was used without further purification in the next step.

### Step 5: Preparation of 1⁵-methyl-1¹H-2,6-diaza-1(2,1)-benzo[d]imidazola-4(1,3)-benzenacycloundecaphane-3,5-dione

To a stirred solution of 3-((1-(5-aminopentyl)-5-methyl-1H-benzo[d]imidazol-2-yl)carbamoyl)benzoic acid (from step 4) in MeCN was added TEA (27 μl, 0.19 mmol) and HATU (24 mg, 0.063 mmol). The reaction mixture was heated to 50° C. and stirred over night. The reaction mixture was diluted with DMSO/dioxane/methanol/water, filtered and purified with reverse phase chromatography (Gemini NX-C18, 21*150 mm, 50 mM NH₄OH (aq)/acetonitrile, gradient over to 12 minutes, 25 ml/min). The pure fractions were pooled and concentrated to give the product as a white solid (9 mg, 39% yield). ¹H NMR (DMSO-d₆) δ: 12.41 (s, 1H), 9.03 (s, 1H), 8.09 (d, 1H), 7.87 (tm, 1H), 7.73 (d, 1H), 7.57 (m, 1H), 7.38 (d, 1H), 7.29 (s, 1H), 7.06 (d, 1H), 4.25 (m, 2H), 3.23-3.13 (m, 2H), 2.38 (s, 3H), 2.03 (m, 2H), 1.81 (m, 2H), 1.42 (m, 2H). LC/MS: M+H=363.2

### Example 5: 1¹H-9-oxa-2,6-diaza-1(2,1)-benzo[d]imidazola-4(1,3)-benzenacycloundecaphane-3,5-dione

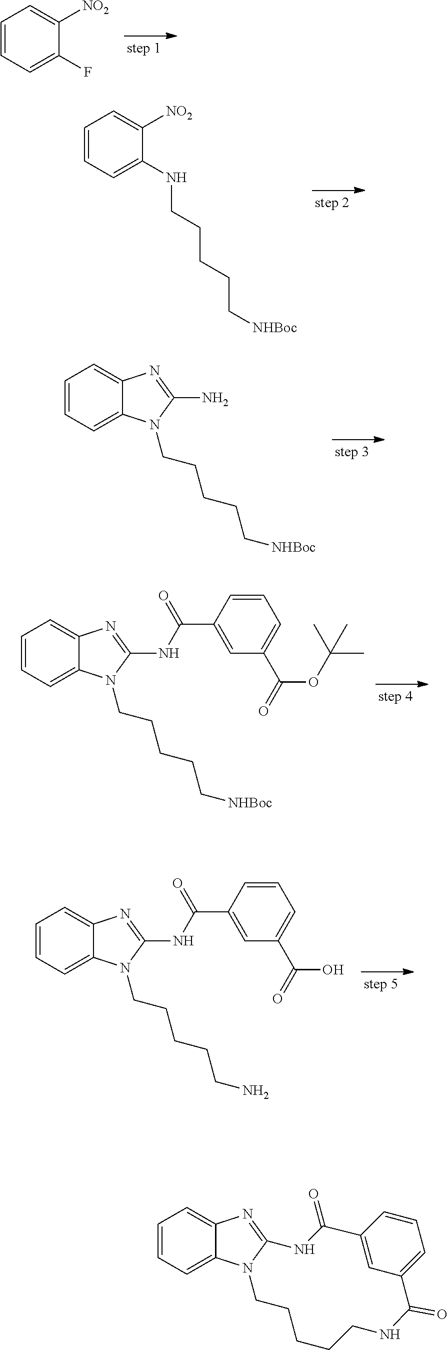

### Step 1: Preparation of tert-butyl (2-(2-((2-nitrophenyl)amino)ethoxy)ethyl)carbamate

A solution of 1-fluoro-2-nitrobenzene (28 mg, 0.2 mmol) and tert-butyl (2-(2-aminoethoxy)ethyl)carbamate (49 mg, 0.24 mmol) and K₂CO₃ (83 mg, 0.6 mmol) in 2 ml MeCN was heated at 80° C. over night. The reaction mixture was cooled to room temperature and the solvent was removed under reduced pressure. The residue partitioned between water and EtOAc. The organic phase was washed with water and concentrated to give the product as a yellow oil (54 mg, 83% yield). ¹H NMR (DMSO-d₆) δ: 8.17 (m, 1H), 8.06 (m, 1H), 7.57-7.51 (m, 1H), 7.09 (d, 1H), 6.75 (m, 1H), 6.73-6.66 (m, 1H), 3.65 (m, 2H), 3.50 (m, 2H), 3.44 (m, 2H), 3.09 (m, 2H), 1.36 (s, 9H). LC/MS: M+Na=348.1

### Step 2: Preparation of tert-butyl (2-(2-(2-amino-1H-benzo[d]imidazol-1-yl)ethoxy)ethyl)carbamate

To a stirred solution of tert-butyl (2-(2-((2-nitrophenyl)amino)ethoxy)ethyl)carbamate (50 mg, 0.15 mmol) in 10 ml EtOH under a nitrogen atmosphere was added 5% Pd/C (8 mg, 0.007 mmol). The nitrogen was replaced by hydrogen and the reaction was stirred at room temperature for 2 hours. The reaction mixture was filtered through celite and washed with MeOH. The filtrate was concentrated under vacuum. The residue was dissolved in 10 ml water/MeOH/MeCN (1:3:1) and CNBr (21 mg, 0.2 mmol) was added at room temperature. The resulting solution was heated at 40° C. in a sealed flask overnight. The reaction mixture was diluted with 50 ml Na₂CO₃ (saturated aqueous solution) and extracted with EtOAc (3×30 ml). The organic phases were combined, dried and concentrated. The crude product was used directly in the following step without further purification. ¹H NMR (DMSO-d₆) δ: 12.59 (s, 1H), 8.46 (s, 2H), 7.56-7.50 (m, 1H), 7.37 (m, 1H), 7.29-7.23 (m, 2H), 6.70 (m, 1H), 4.28 (m, 2H), 3.70 (m, 2H), 3.41-3.36 (Overlap with DMSO, m, 2H), 3.00 (m, 2H), 1.35 (s, 9H). LC/MS: M+H=321.2

### Step 3: Preparation of tert-butyl 3-((1-(2-(2-((tert-butoxycarbonyl)amino)ethoxy)ethyl)-1H-benzo[d]imidazol-2-yl)carbamoyl)benzoate

To a stirred solution of tert-butyl (2-(2-(2-amino-1H-benzo[d]imidazol-1-yl)ethoxy)ethyl)carbamate (65 mg, 0.16 mmol) in 5 ml MeCN was added 3-(tert-butoxycarbonyl)benzoic acid (36 mg, 0.16 mmol), HATU (61 mg, 0.16 mmol) and TEA (68 μl, 0.49 mmol). The reaction mixture was stirred at 50° C. over night. The reaction mixture was cooled to room temperature and the solvent was removed under reduced pressure. The residue partitioned between water and ethyl acetate and the organic phase was washed with 0.1 M NaOH, 0.1 M HCl, sat. NaHCO₃ and brine, dried over MgSO₄, filtered and concentrated. The crude product (74 mg) was used directly in the following step without further purification. ¹H NMR (DMSO-d₆) δ: 12.79 (s, 1H), 8.77 (s, 1H), 8.44 (d, 1H), 8.03 (d, 1H), 7.59 (m, 1H), 7.58-7.50 (m, 2H), 7.30-7.18 (m, 2H), 6.59 (m, 1H), 4.43 (m, 2H), 3.84 (m, 2H), 3.47-3.41 (overlap with water peak, m, 2H), 2.97 (m, 2H), 1.58 (s, 9H), 1.31 (s, 9H). LC/MS: M+H=523.3.

### Step 4: Preparation of 3-((1-(2-(2-aminoethoxy)ethyl)-1H-benzo[d]imidazol-2-yl)carbamoyl)benzoic acid

tert-butyl 3-((1-(2-(2-((tert-butoxycarbonyl)amino)ethoxy)ethyl)-1H-benzo[d]imidazol-2-yl)carbamoyl)benzoate (74 mg, 0.14 mmol) was dissolved in 2 ml dioxane and 1 ml water and 1 ml HCl (conc) was added. The reaction mixture was stirred over night. The reaction mixture was concentrated. The residue was dissolved in toluene and concentrated to dryness under vacuum. The product was used without further purification in the next step.

### Step 5: Preparation of 1¹H-9-oxa-2,6-diaza-1(2,1)-benzo[d]imidazola-4(1,3)-benzenacycloundecaphane-3,5-dione

To a stirred solution of 3-((1-(2-(2-aminoethoxy)ethyl)-1H-benzo[d]imidazol-2-yl)carbamoyl)benzoic acid (from previous step) in 10 ml MeCN was added HATU (80 mg, 0.21 mmol) and TEA (59 μl, 0.42 mmol). The reaction mixture was heated at 50° C. over night. The reaction mixture was cooled to room temperature and diluted with DMSO/dioxane/methanol/water, filtered and purified with reverse phase chromatography (Gemini NX-C18, 21*150 mm, 50 mM NH₄OH (aq)/acetonitrile, gradient over to 12 minutes, 25 ml/min). The pure fractions were pooled and concentrated to give the product as a white solid (8 mg, 16% yield). ¹H NMR (DMSO-d₆) δ: 12.35 (s, 1H), 9.42 (s, 1H), 8.06 (d, 1H), 7.70 (d, 2H), 7.55 (m, 1H), 7.48 (m, 2H), 7.20 (m, 2H), 4.47 (m, 2H), 3.83 (m, 2H), 3.76 (m, 2H). LC/MS: M+H=351.1

### Example δ: 1′-methyl-1¹H-2,6-diaza-1(2,1)-benzo[d]imidazola-4(1,3)-benzenacycloundecaphane-3,5-dione

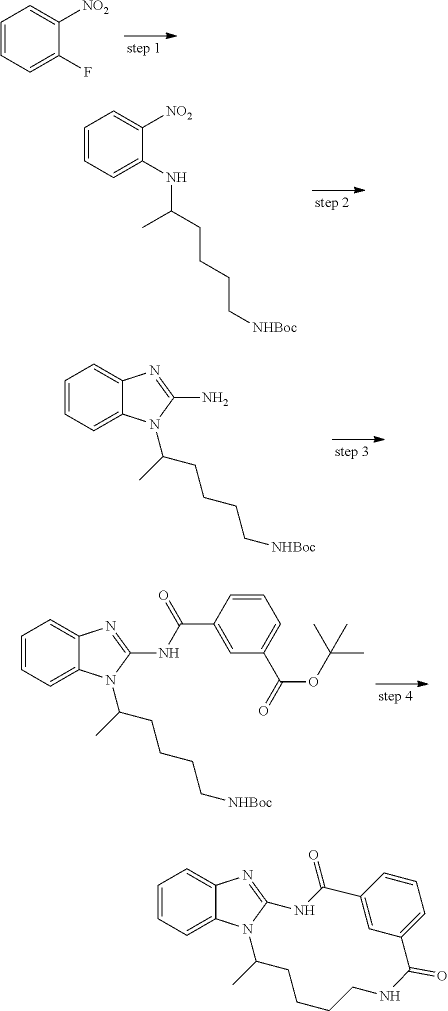

### Step 1: Preparation of tert-butyl (5-((2-nitrophenyl)amino)hexyl)carbamate

A solution of 1-fluoro-2-nitrobenzene (42 mg, 0.3 mmol) and tert-butyl (5-aminohexyl)carbamate (78 mg, 0.36 mmol) and K₂CO₃ (124 mg, 0.9 mmol) in 50 ml MeCN was heated at 80° C. over night. The reaction mixture was cooled to room temperature and the solvent was removed under reduced pressure. The residue partitioned between water and EtOAc. The organic phase was washed with water, 0.1 M HCl, NaHCO₃ (sat) and brine, dried and concentrated to give the product as a yellow oil (91 mg, 83% yield). ¹H NMR (DMSO-d₆) δ: 8.06 (m, 1H), 7.91 (d, 1H), 7.53 (m, 1H), 7.09 (d, 1H), 6.76 (m, 1H), 6.67 (m, 1H), 3.80 (m, 1H), 2.90 (m, 2H), 1.67-1.49 (m, 2H), 1.44-1.26 (m, 9H), 1.21 (d, 3H). LC/MS: M+Na=360.2

### Step 2: Preparation of tert-butyl (5-(2-amino-1H-benzo[d]imidazol-1-yl)hexyl)carbamate

To a stirred solution of tert-butyl (5-((2-nitrophenyl)amino)hexyl)carbamate (91 mg, 0.27 mmol) in 5 ml EtOH under a nitrogen atmosphere was added 10% Pd/C (14 mg, 0.013 mmol). The nitrogen was replaced by hydrogen and the reaction was stirred at room temperature under hydrogen for 4 hours. The reaction mixture was filtered through celite and washed with MeOH. The filtrate was concentrated under vacuum. The residue was dissolved in 10 ml water/MeOH/MeCN (1:3:1) and CNBr (37 mg, 0.35 mmol) was added at room temperature. The resulting solution was heated at 40° C. in a sealed flask over night. The reaction mixture was diluted with 50 ml Na₂CO₃ (saturated aqueous solution) and extracted with EtOAc (3×30 ml). The organic phases were combined, dried and concentrated. The crude product (87 mg) was used directly in the following step without further purification. ¹H NMR (DMSO-d₆) δ: 7.28 (d, 1H), 7.12 (d, 1H), 6.91 (m, 1H), 6.83 (m, 1H), 6.70 (m, 1H), 6.32 (s, 2H), 4.37 (h, J=6.7 Hz, 1H), 2.81 (m, 2H), 2.06-1.93 (m, 1H), 1.83-1.71 (m, 1H), 1.44 (d, 3H), 1.34 (s, 9H), 1.24-1.11 (m, 1H), 1.03-0.90 (m, 1H); LC/MS: M+H=333.2.

### Step 3: Preparation of tert-butyl 3-((1-(6-((tert-butoxycarbonyl)amino)hexan-2-yl)-1H-benzo[d]imidazol-2-yl)carbamoyl)benzoate

To a stirred solution of tert-butyl (5-(2-amino-1H-benzo[d]imidazol-1-yl)hexyl)carbamate (33 mg, 0.10 mmol) in 0.4 ml MeCN was added 3-(tert-butoxycarbonyl)benzoic acid (22 mg, 0.10 mmol), HATU (38 mg, 0.10 mmol) and TEA (28 μl, 0.20 mmol). The reaction mixture was stirred at 50° C. over night. The reaction mixture was cooled to room temperature and the reaction mixture was diluted with methanol/water and purified with reverse phase chromatography (Gemini NX-C18, 21*150 mm, 50 mM NH₄OH (aq)/acetonitrile, gradient over to 12 minutes, 25 ml/min). The pure fractions were pooled and concentrated to give the product as a white solid (25 mg, 47% yield). ¹H NMR (DMSO-d₆) δ: 12.84 (s, 1H), 8.79 (s, 1H), 8.41 (d, 1H), 8.03 (d, 1H), 7.66-7.55 (m, 3H), 7.27-7.20 (m, 2H), 6.63 (m, 1H), 5.09 (s, 1H), 2.81 (m, 2H), 2.27 (s, 1H), 1.95-1.85 (m, 1H), 1.63-1.55 (m, 9H), 1.46-1.33 (m, 2H), 1.30-1.17 (s, 9H), 1.13-1.00 (m, 1H). LC/MS: M+H=537.3

### Step 4: Preparation of 11-methyl-1¹H-2,6-diaza-1(2,1)-benzo[d]imidazola-4(1,3)-benzenacycloundecaphane-3,5-dione

tert-butyl 3-((1-(6-((tert-butoxycarbonyl)amino)hexan-2-yl)-1H-benzo[d]imidazol-2-yl)carbamoyl)benzoate (23 mg, 0.043 mmol) was dissolved in 2 ml dioxane and 1 ml water and 1 ml HCl (conc) was added. The reaction mixture was stirred over night. The reaction mixture was concentrated. The residue was dissolved in toluene and concentrated to dryness under vacuum. The residue was dissolved in 10 ml MeCN and to the resulting solution was added HATU (24 mg, 0.06 mmol) and TEA (18 μl, 0.13 mmol). The reaction mixture was heated at 50° C. over night. The reaction mixture was cooled to room temperature and diluted with DMSO/dioxane/methanol/water, filtered and purified with reverse phase chromatography (Gemini NX-C18, 21*150 mm, 50 mM NH₄OH (aq)/acetonitrile, gradient over to 12 minutes, 25 ml/min). The pure fractions were pooled and concentrated to give the product as a white solid (6 mg, 39% yield). ¹H NMR (DMSO-d₆) δ: 12.43 (s, 1H), 9.02 (s, 1H), 8.10 (d, 1H), 7.88 (m, 1H), 7.73 (d, 1H), 7.61-7.54 (m, 2H), 7.54-7.46 (m, 1H), 7.24-7.14 (m, 2H), 5.46-5.33 (m, 1H), 3.19-2.98 (m, 2H), 2.29-2.15 (m, 2H), 1.91-1.76 (m, 1H), 1.66 (d, 3H), 1.64-1.58 (m, 1H), 1.42-1.30 (m, 1H), 1.24-1.12 (m, 1H); LC/MS: M+H=363.1

### Example 7: methyl 1′-methyl-3,5-dioxo-1¹H-2,6-diaza-1(2,1)-benzo[d]imidazola-4(1,3)-benzenacycloundecaphane-1⁷-carboxylate

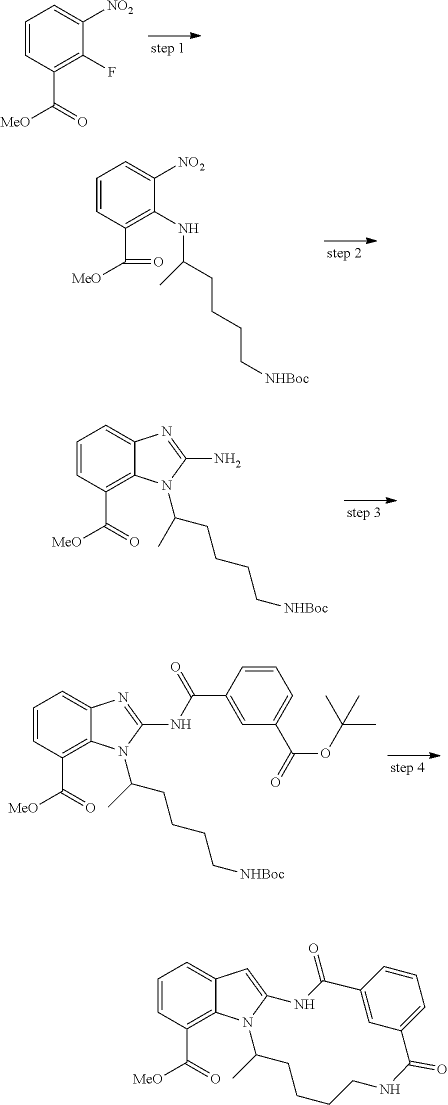 [SMILES: COC(=O)c1cccc([N+](=O)[O-])c1F ; COC(=O)c1cccc([N+](=O)[O-])c1NC(C)CCCCNC(=O)OC(C)(C)C ; COC(=O)c1cccc2nc(N)n(C(C)CCCCNC(=O)OC(C)(C)C)c12 ; COC(=O)c1cccc2nc(NC(=O)c3cccc(C(=O)OC(C)(C)C)c3)n(C(C)CCCCNC(=O)OC(C)(C)C)c12 ; COC(=O)c1cccc2cc3n(c12)C(C)CCCCNC(=O)c1cccc(c1)C(=O)N3]

### Step 1: Preparation of methyl 2-((6-((tert-butoxycarbonyl)amino)hexan-2-yl)amino)-3-nitrobenzoate

A solution of methyl 2-fluoro-3-nitrobenzoate (300 mg, 1.51 mmol), tert-butyl (5-aminohexyl)carbamate (391 mg, 1.81 mmol) and K₂CO₃ (625 mg, 4.52 mmol) in 50 ml MeCN was heated at 80° C. overnight. The reaction mixture was cooled to room temperature and the solvent was removed under reduced pressure. The residue partitioned between water and EtOAc. The organic phase was washed with water, 0.1 M HCl, NaHCO₃ (sat) and brine, dried and concentrated to give the product as a yellow oil (580 mg, 97% yield). ¹H NMR (DMSO-d₆) δ: 8.04 (m, 1H), 8.01-7.96 (m, 2H), 6.83 (m, 1H), 6.72 (m, 1H), 3.86 (s, 3H), 3.20-3.11 (m, 1H), 2.84 (m, 2H), 1.45-1.38 (m, 2H), 1.35 (s, 9H), 1.33-1.25 (m, 2H), 1.24-1.14 (m, 2H), 1.05 (d, 3H); LC/MS: M+Na=418.1

### Step 2: Preparation of methyl 2-amino-1-(6-((tert-butoxycarbonyl)amino)hexan-2-yl)-1H-benzo[d]imidazole-7-carboxylate

To a stirred solution methyl 2-((6-((tert-butoxycarbonyl)amino)hexan-2-yl)amino)-3-nitrobenzoate (580 mg, 1.47 mmol) in 10 ml MeOH under a nitrogen atmosphere was added 10% Pd/C (14 mg, 0.013 mmol). The nitrogen was replaced by hydrogen and the reaction was stirred at room temperature under hydrogen for 2 hours. The reaction mixture was filtered through celite and washed with MeOH. The filtrate was concentrated under vacuum. The residue was dissolved in 10 ml water/MeOH/MeCN (1:3:1) and CNBr (202 mg, 1.91 mmol) was added at room temperature. The resulting solution was heated at 40° C. in a sealed flask over night. The reaction mixture was diluted with 50 ml Na₂CO₃ (saturated aqueous solution) and extracted with EtOAc (3×30 ml). The organic phases were combined, dried and concentrated. The crude product (550 mg) was used directly in the following step without further purification. ¹H NMR (DMSO-d₆) δ: 7.29 (m, 1H), 7.16 (m, 1H), 6.98 (m, 1H), 6.68 (m, 1H), 6.31 (s, 2H), 4.44 (m, 1H), 3.86 (s, 3H), 2.79 (m, 2H), 2.02-1.91 (m, 1H), 1.81-1.70 (m, 1H), 1.48 (d, J=7.0 Hz, 3H), 1.39-1.23 (m, 1¹H), 1.07-0.94 (m, 1H), 0.90-0.76 (m, 1H); LC/MS: M+H=391.2

### Step 3: Preparation of methyl 1-(6-((tert-butoxycarbonyl)amino)hexan-2-yl)-2-(3-(tert-butoxycarbonyl)benzamido)-1H-benzo[d]imidazole-7-carboxylate

To a stirred solution of methyl 2-amino-1-(6-((tert-butoxycarbonyl)amino)hexan-2-yl)-1H-benzo[d]imidazole-7-carboxylate (350 mg, 0.90 mmol) in 10 ml MeCN was added 3-(tert-butoxycarbonyl)benzoic acid (199 mg, 0.90 mmol), HATU (340 mg, 0.90 mmol) and TEA (250 μl, 1.79 mmol). The reaction mixture was stirred at 50° C. over night. The reaction mixture was cooled to room temperature and the solvent was removed under reduced pressure. The residue was partitioned between water and EtOAc and the organic phase was washed with 0.1M NaOH, 0.1M HCl, NaHCO₃ (sat.) and brine, dried and concentrated. The crude product was purified by flash chromatography (SiO₂, 10-30% EtOAc/petroleum ether) to give the product as a light brown oil (390 mg, 73% yield). ¹H NMR (DMSO-d₆) δ: 8.82 (s, 1H), 8.37 (d, 1H), 8.05 (d, 1H), 7.75 (d, 1H), 7.63 (m, 1H), 7.50 (d, 1H), 7.29 (m, 1H), 6.61 (m, 1H), 3.94 (s, 3H), 2.76 (m, 2H), 1.74 (d, 3H), 1.58 (s, 9H), 1.28 (s, 9H). LC/MS: M+H=595.3

### Step 4: Preparation of methyl 11-methyl-3,5-dioxo-1¹H-2,6-diaza-1(2,1)-benzo[d]imidazola-4(1,3)-benzenacycloundecaphane-17-carboxylate

Methyl 1-(6-((tert-butoxycarbonyl)amino)hexan-2-yl)-2-(3-(tert-butoxycarbonyl)benzamido)-1H-benzo[d]imidazole-7-carboxylate (390 mg, 0.66 mmol) was dissolved in 5 ml dioxane and 2 ml water and 2 ml HCl (conc) was added. The reaction mixture was stirred over night. The reaction mixture was concentrated. The residue was dissolved in toluene and concentrated to dryness under vacuum. The residue was dissolved in 300 ml dioxane and to the resulting solution was added HATU (298 mg, 0.79 mmol) and TEA (573 μl, 3.28 mmol). The reaction mixture was heated at room temperature over night. The reaction mixture was cooled to room temperature and the solvent was removed under reduced pressure. The residue was partitioned between water and EtOAc and the organic phase was washed with 0.1M NaOH, 0.1 M HCl, NaHCO₃ (sat.) and brine, dried and concentrated. The crude product was purified by flash chromatography (SiO₂, 100% EtOAc) to give the product as a light brown oil (58 mg, 21% yield). ¹H NMR (DMSO-d₆) δ: 12.86 (s, 1H), 8.81 (s, 1H), 8.11 (d, 1H), 7.83-7.69 (m, 3H), 7.60 (m, 1H), 7.50 (d, 1H), 7.28 (m, 1H), 4.93 (bs, 1H), 3.93 (s, 3H), 3.49 (bs, 1H), 3.10 (bs, 1H), 2.91 (bs, 1H), 1.90 (bs, 3H), 1.67-1.56 (m, 4H), 1.45 (bs, 1H). LC/MS: M+H=421.2

### Example 8: 1′-methyl-1⁷-(4-methylpiperazine-1-carbonyl)-1¹H-2,6-diaza-1(2,1)-benzo[d]imidazola-4(1,3)-benzenacycloundecaphane-3,5-dione

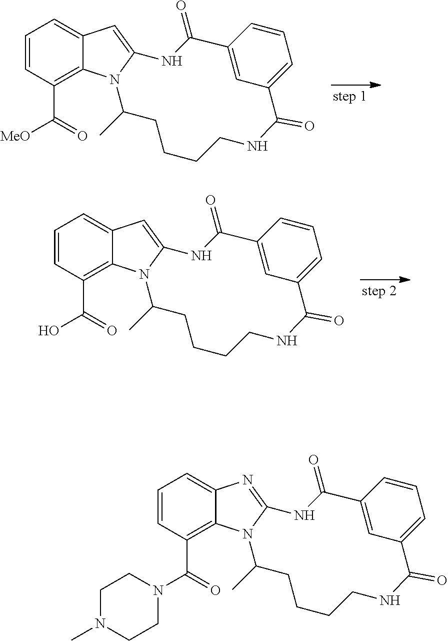

### Step 1: Preparation of 11-methyl-3,5-dioxo-1¹H-2,6-diaza-1(2,1)-benzo[d]imidazola-4(1,3)-benzenacycloundecaphane-1⁷-carboxylic acid

A solution of methyl 11-methyl-3,5-dioxo-1¹H-2,6-diaza-1(2,1)-benzo[d]imidazola-4(1,3)-benzenacycloundecaphane-1⁷-carboxylate (55 mg, 0.13 mmol) and NaOH (37 mg, 0.92 mmol) in 10 ml water was stirred at room temperature over night. The reaction mixture was acidified with 2 ml 1M HCl. The precipitate was collected and washed with water. The product was used in the next step without further purification. LC/MS: M+H=407.1, Rₜ: 4.0 min

### Step 2: Preparation of 11-methyl-1⁷-(4-methylpiperazine-1-carbonyl)-11H-2,6-diaza-1(2,1)-benzo[d]imidazola-4(1,3)-benzenacycloundecaphane-3,5-dione

A solution of 11-methyl-3,5-dioxo-1¹H-2,6-diaza-1(2,1)-benzo[d]imidazola-4(1,3)-benzenacycloundecaphane-1⁷-carboxylic acid (25 mg, 0.062 mmol), methylpiperazine (9 mg, 0.092 mmol), HATU (23 mg, 0.062 mmol) and TEA (26 μl, 0.19 mmol) was stirred over night at 50° C. The reaction mixture was diluted with water and MeOH and purified with reverse phase chromatography (Gemini NX-C18, 21*150 mm, 50 mM NH₄OH (aq)/acetonitrile, gradient over to 12 minutes, 25 ml/min). The pure fractions were pooled and concentrated to give the product as a white solid (15 mg, 50% yield). ¹H NMR (DMSO-d₆) δ: 12.62 (bs, 1H), 8.77 (s, 1H), 8.11 (d, 1H), 7.74 (d, 1H), 7.62-7.52 (m, 2H), 7.22 (m, 1H), 7.03 (d, 1H), 4.56 (bs, 1H), 4.15-2.91 (m, 10H), 3.29 (s, 3H), 2.19 (d, 3H), 2.66-1.33 (m, 6H). LC/MS: M+H=489.2

### Example 9: tert-butyl 4-(11-methyl-3,5-dioxo-1¹H-2,6-diaza-1(2,1)-benzo[d]imidazola-4(1,3)-benzenacycloundecaphane-1⁷-carbonyl)piperazine-1-carboxylate

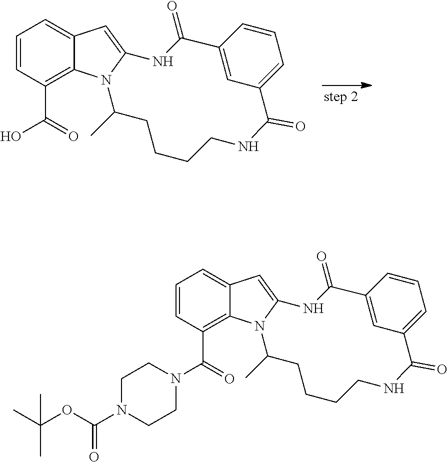 [SMILES: CC1CCCCNC(=O)c2cccc(c2)C(=O)Nc2cc3cccc(C(=O)O)c3n21 ; CC1CCCCNC(=O)c2cccc(c2)C(=O)Nc2cc3cccc(C(=O)N4CCN(C(=O)OC(C)(C)C)CC4)c3n21]

### Step 1: Preparation of tert-butyl 4-(11-methyl-3,5-dioxo-1¹H-2,6-diaza-1(2,1)-benzo[d]imidazola-4(1,3)-benzenacycloundecaphane-1⁷-carbonyl)piperazine-1-carboxylate

A solution of 11-methyl-3,5-dioxo-1¹H-2,6-diaza-1(2,1)-benzo[d]imidazola-4(1,3)-benzenacycloundecaphane-1⁷-carboxylic acid (25 mg, 0.062 mmol), tert-butyl piperazine-1-carboxylate (17 mg, 0.092 mmol), HATU (23 mg, 0.062 mmol) and TEA (26 μl, 0.19 mmol) was stirred over night at 50° C. The reaction mixture was diluted with water and MeOH and purified with reverse phase chromatography (Gemini NX-C18, 21*150 mm, 50 mM NH₄OH (aq)/acetonitrile, gradient over to 12 minutes, 25 ml/min). The pure fractions were pooled and concentrated to give the product as an off-white white solid (18 mg, 51% yield). ¹H NMR (DMSO-d₆) δ: 12.70 (bs, 1H), 8.77 (bs, 1H), 8.11 (d, 1H), 7.86-7.76 (m, 1H), 7.75 (d, 1H), 7.64-7.53 (m, 2H), 7.24 (m, 1H), 7.09 (m, 1H), 4.53 (b s, 1H), 3.90-2.91 (overlap with water peak, m, 10H), 2.07-1.44 (m, 9H), 1.40 (s, 9H); LC/MS: M+H=575.3.

### Example 10: 1⁷-(4-methylpiperazine-1-carbonyl)-1¹H-2,6-diaza-1(2,1)-benzo[d]imidazola-4(1,3)-benzenacycloundecaphane-3,5-dione

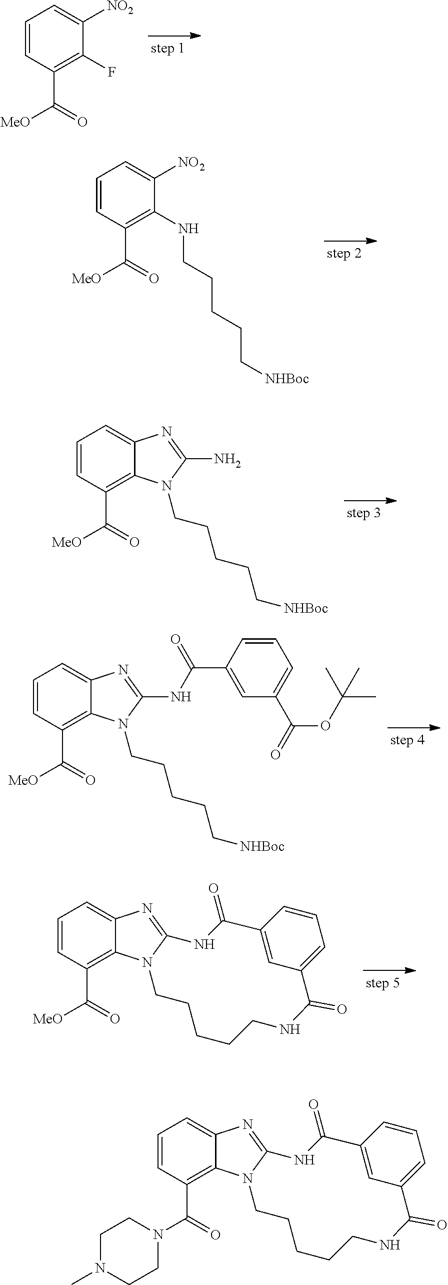

### Step 1: Preparation of methyl 2-((5-((tert-butoxycarbonyl)amino)pentyl)amino)-3-nitrobenzoate

A solution of methyl 2-fluoro-3-nitrobenzoate (500 mg, 2.51 mmol), tert-butyl (5-aminopentyl)carbamate (610 mg, 3.01 mmol) and K₂CO₃ (1.05 g, 7.53 mmol) in 20 ml MeCN was heated at 80° C. over night. The reaction mixture was cooled to room temperature and the solvent was removed under reduced pressure. The residue partitioned between water and EtOAc. The organic phase was washed with water, 0.1 M HCl, NaHCO₃ (sat) and brine, dried and concentrated to give the product as a yellow oil (960 mg, 100% yield). ¹H NMR (DMSO-d₆) δ: 8.24 (m, 1H), 8.06-8.02 (m, 2H), 6.82-6.72 (m, 2H), 3.86 (s, 3H), 2.91-2.81 (m, 4H), 1.56 (m, 2H), 1.38-1.32 (m, 1¹H), 1.31-1.22 (m, 2H); LC/MS: M+H=382.2

### Step 2: Preparation of methyl 2-amino-1-(5-((tert-butoxycarbonyl)amino)pentyl)-1H-benzo[d]imidazole-7-carboxylate

To a stirred solution methyl methyl 2-((5-((tert-butoxycarbonyl)amino)pentyl)amino)-3-nitrobenzoate (500 mg, 1.31 mmol) in 10 ml MeOH under a nitrogen atmosphere was added 5% Pd/C (70 mg, 0.066 mmol). The nitrogen was replaced by hydrogen and the reaction was stirred at room temperature under hydrogen for 2 hours. The reaction mixture was filtered through celite and washed with MeOH. The filtrate was concentrated under vacuum. The residue was dissolved in 10 ml water/MeOH/MeCN (1:3:1) and CNBr (181 mg, 1.70 mmol) was added at room temperature. The resulting solution was heated at 40° C. in a sealed flask over night. The reaction mixture was diluted with 50 ml NaHCO₃ (saturated aqueous solution) and extracted with EtOAc (3×30 ml). The organic phases were combined, washed with brine and NaHCO₃(sat) dried and concentrated. The crude product (550 mg) was used directly in the following step without further purification. ¹H NMR (DMSO-d₆) δ: 7.33 (m, 1H), 7.24 (m, 1H), 7.00 (m, 1H), 6.73 (m, 1H), 6.56 (s, 2H), 4.12 (m, 2H), 3.87 (s, 3H), 2.85 (m, 2H), 1.43 (m, 2H), 1.38-1.29 (m, 1¹H), 1.21-1.10 (m, 2H). LC/MS: M+H=377.2

### Step 3: Preparation of methyl 1-(5-((tert-butoxycarbonyl)amino)pentyl)-2-(3-(tert-butoxycarbonyl)benzamido)-1H-benzo[d]imidazole-7-carboxylate

To a stirred solution of methyl 2-amino-1-(6-((tert-butoxycarbonyl)amino)hexan-2-yl)-1H-benzo[d]imidazole-7-carboxylate (500 mg, 1.33 mmol) in 10 ml MeCN was added 3-(tert-butoxycarbonyl)benzoic acid (295 mg, 1.33 mmol), HATU (504 mg, 1.33 mmol) and TEA (555 μl, 3.98 mmol). The reaction mixture was stirred at 50° C. over night. The reaction mixture was cooled to room temperature and the solvent was removed under reduced pressure. The residue was partitioned between water and EtOAc and the organic phase was washed with 0.1 M NaOH, 0.1M HCl, NaHCO₃(sat.) and brine, dried and concentrated. The crude product was purified by flash chromatography (SiO₂, 10-30% EtOAc/petroleum ether) to give the product as a light brown oil (530 mg, 69% yield). ¹H NMR (DMSO-d₆) δ: 13.10 (s, 1H), 8.79 (s, 1H), 8.45 (d, 1H), 8.05 (d, 1H), 7.80 (d, 1H), 7.65-7.57 (m, 2H), 7.32 (m, 1H), 6.69 (m, 1H), 4.47 (m, 2H), 3.94 (s, 3H), 2.87 (m, 2H), 1.67-1.60 (m, 2H), 1.59 (s, 9H), 1.42 (m, 2H), 1.32 (s, 9H), 1.31-1.25 (m, 2H); LC/MS: M+H=581.3.

### Step 4: Preparation of methyl 3,5-dioxo-1¹H-2,6-diaza-1(2,1)-benzo[d]imidazola-4(1,3)-benzenacycloundecaphane-1⁷-carboxylate

Methyl 1-(5-((tert-butoxycarbonyl)amino)pentyl)-2-(3-(tert-butoxycarbonyl)benzamido)-1H-benzo[d]imidazole-7-carboxylate (530 mg, 0.91 mmol) was dissolved in 5 ml dioxane and 5 ml 5 M HCl was added. The reaction mixture was stirred over night at 35° C. The reaction mixture was concentrated. The residue (LC/MS: M+H=425.2) was dissolved in 150 ml THF and to the resulting solution was added HATU (415 mg, 1.10 mmol) and DIPEA (797 μl, 4.56 mmol). The reaction mixture was stirred at room temperature over night. The reaction mixture was cooled to room temperature and the solvent was removed under reduced pressure. The residue was dissolved in water/MeCN at 80° C. After cooling to room temperature the precipitate was collected by filtration. The collected product (280 mg, 76% yield) was used without further purification.

### Step 5: Preparation of 1⁷-(4-methylpiperazine-1-carbonyl)-1¹H-2,6-diaza-1(2,1)-benzo[d]imidazola-4(1,3)-benzenacycloundecaphane-3,5-dione

To a stirred solution of methyl 3,5-dioxo-1¹H-2,6-diaza-1(2,1)-benzo[d]imidazola-4(1,3)-benzenacycloundecaphane-1⁷-carboxylate (41 mg, 0.1 mmol) in 4 ml dioxane/water (1:1) was added NaOH (20 mg, 0.5 mmol). The reaction mixture was stirred over night at room temperature. The solution was acidified with 1 ml 1 M HCl and the reaction was concentrated to dryness through repeated addition and evaporation of MeCN and toluene. The residue was dissolved in 5 ml MeCN and HATU (38 mg, 0.15 mmol) and TEA (42 μl, 0.3 mmol) were added. The reaction mixture was heated to 50° C. and stirred over night. The reaction mixture was cooled to room temperature, diluted with DMSO/methanol/water, filtered and purified with reverse phase chromatography (Gemini NX-C18, 21*150 mm, 50 mM NH₄OH (aq)/acetonitrile, gradient over to 12 minutes, 25 ml/min). The pure fractions were pooled and concentrated to give the product as a white solid (26 mg, 55% yield). ¹H NMR (DMSO-d₆) δ: 8.92 (s, 1H), 8.11 (d, 1H), 7.76 (m, 1H), 7.64 (d, 1H), 7.54 (d, 1H), 7.50 (m, 1H), 6.97 (bs, 1H), 6.75 (bs, 1H), 4.12 (bs, 2H), 3.78 (bs, 1H), 3.65 (bs, 1H), 3.30-3.20 (m, 4H), 2.47 (bs, 1H), 2.32 (bs, 2H), 2.19 (s, 3H), 2.13 (bs, 1H), 1.94 (bs, 2H), 1.84 (bs, 1H), 1.69 (bs, 1H), 1.43 (bs, 2H); LC/MS: M+H=475.2.

### Example 11: 1⁵-(4-methylpiperazine-1-carbonyl)-1¹H-2,6-diaza-1(2,1)-benzo[d]imidazola-4(1,3)-benzenacycloundecaphane-3,5-dione

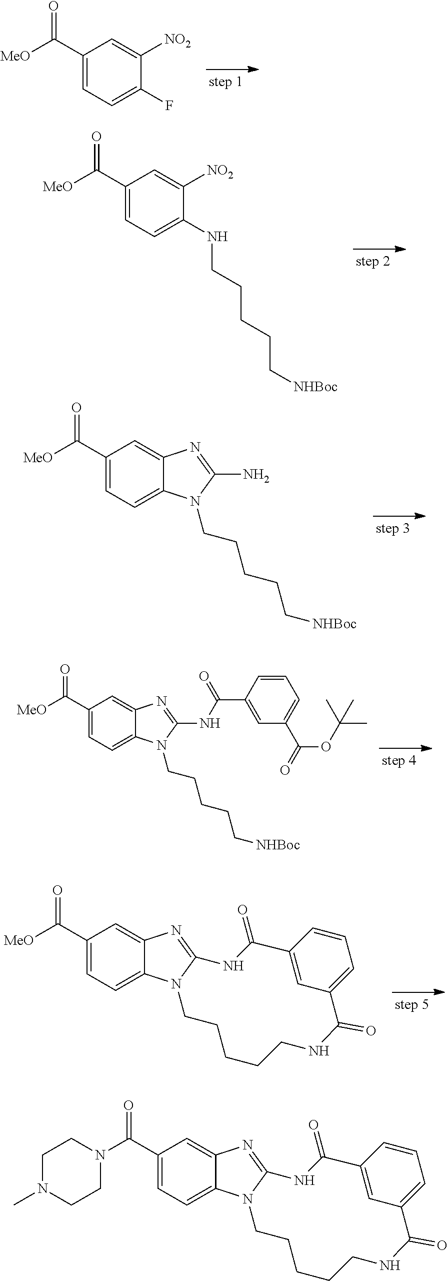

### Step 1: Preparation of methyl 4-((5-((tert-butoxycarbonyl)amino)pentyl)amino)-3-nitrobenzoate

A solution of methyl 4-fluoro-3-nitrobenzoate (755 mg, 3.50 mmol), tert-butyl (5-aminopentyl)carbamate (850 mg, 4.20 mmol) and K₂CO₃ (1.45 g, 10-50 mmol) in 50 ml MeCN was heated at 80° C. over night. The reaction mixture was cooled to room temperature and the solvent was removed under reduced pressure. The residue partitioned between water and EtOAc. The organic phase was washed with water, 0.1 M HCl, NaHCO₃(sat) and brine, dried and concentrated to give the product as a yellow oil (1300 mg, 97% yield). ¹H NMR (DMSO-d₆) δ: 8.62 (d, 1H), 8.53 (m, 1H), 7.96 (m, 1H), 7.15 (d, 1H), 6.77 (m, 1H), 3.82 (s, 3H), 3.40 (m, 2H), 2.91 (m, 2H), 1.62 (m, 2H), 1.46-1.30 (m, 13H); LC/MS: M+Na=404.1 #

### Step 2: Preparation of methyl 2-amino-1-(5-((tert-butoxycarbonyl)amino)pentyl)-1H-benzo[d]imidazole-5-carboxylate

To a stirred solution methyl methyl methyl 4-((5-((tert-butoxycarbonyl)amino)pentyl)amino)-3-nitrobenzoate (700 mg, 1.84 mmol) in 10 ml MeOH under a nitrogen atmosphere was added 5% Pd/C (98 mg, 0.092 mmol). The nitrogen was replaced by hydrogen and the reaction was stirred at room temperature under hydrogen for 2 hours. The reaction mixture was filtered through celite and washed with MeOH. The filtrate was concentrated under vacuum. The residue was dissolved in 10 ml water/MeOH/MeCN (1:3:1) and CNBr (253 mg, 1.70 mmol) was added at room temperature. The resulting solution was heated at 30° C. in a sealed flask over night. The reaction mixture was concentrated under reduced pressure and partitioned between NaHCO₃ (sat.) and EtOAc. The organic phase was washed with brine and NaHCO₃ (sat) dried and concentrated. The crude product (590 mg) was used directly in the following step without further purification. ¹H NMR (DMSO-d₆) δ: 7.69 (d, 1H), 7.57 (m, 1H), 7.23 (d, 1H), 6.75 (m, 1H), 6.65 (s, 2H), 3.97 (m, 2H), 3.81 (s, 3H), 2.88 (m, 2H), 1.61 (m, 2H), 1.43-1.34 (m, 1¹H), 1.31-1.22 (m, 2H); LC/MS: M+H=377.2

### Step 3: Preparation of methyl 1-(5-((tert-butoxycarbonyl)amino)pentyl)-2-(3-(tert-butoxycarbonyl)benzamido)-1H-benzo[d]imidazole-5-carboxylate

To a stirred solution of methyl 2-amino-1-(5-((tert-butoxycarbonyl)amino)pentyl)-1H-benzo[d]imidazole-5-carboxylate (590 mg, 1.57 mmol) in 10 ml MeCN was added 3-(tert-butoxycarbonyl)benzoic acid (348 mg, 1.57 mmol), HATU (594 mg, 1.57 mmol) and TEA (655 μl, 4.70 mmol). The reaction mixture was stirred at 50° C. over night. The reaction mixture was cooled to room temperature and the solvent was removed under reduced pressure. The residue was partitioned between water and EtOAc and the organic phase was washed with 0.1M NaOH, 0.1M HCl, NaHCO₃ (sat.) and brine, dried and concentrated. The crude product was purified by flash chromatography (SiO₂, 10-30% EtOAc/petroleum ether) to give the product as a light brown oil (610 mg, 67% yield). ¹H NMR (DMSO-d₆) δ: 12.96 (s, 1H), 8.79 (s, 1H), 8.44 (d, 1H), 8.13 (s, 1H), 8.05 (d, 1H), 7.89 (d, 1H), 7.67-7.58 (m, 2H), 6.69 (m, 1H), 4.28 (m, 2H), 3.87 (s, 3H), 2.88 (m, 2H), 1.83 (m, 2H), 1.59 (s, 9H), 1.45 (m, 2H), 1.38-1.32 (m, 2H), 1.31 (s, 9H); LC/MS: M+H=581.3.

### Step 4: Preparation of 3,5-dioxo-1¹H-2,6-diaza-1(2,1)-benzo[d]imidazola-4(1,3)-benzenacycloundecaphane-1⁵-carboxylic acid

Methyl 1-(5-((tert-butoxycarbonyl)amino)pentyl)-2-(3-(tert-butoxycarbonyl)benzamido)-1H-benzo[d]imidazole-5-carboxylate (610 mg, 1.05 mmol) was dissolved in 5 ml dioxane and 2 ml water and 2 ml HCl (conc.) was added. The reaction mixture was stirred over night at room temperature. The reaction mixture was concentrated. The residue was dissolved in 150 ml dioaxane and to the resulting solution was added HATU (478 mg, 1.26 mmol) and DIPEA (918 μl, 5.25 mmol). The reaction mixture was stirred at room temperature over night. The reaction mixture was cooled to room temperature and the solvent was removed under reduced pressure. The residue was partitioned between water and EtOAc. The organic phase was washed with 0.1 M NaOH, 0.1 M HCl, NaHCO₃ (sat.) and brine, dried, filtered and concentrated. An insoluble residue was collected from the combined aqueous phase and washed with water. The insoluble residue was mixed in 5 ml of dioxane and 5 ml of water. Sodium hydroxide (126 mg, 3.15 mmol) was added and the reaction mixture stirred overnight. The reaction mixture was acidified with 5 M of 1 M HCl and the precipitate was collected by centrifugation, washed repeatedly with water and dried in vacuum. The collected product (140 mg) was used without further purification. LC/MS: M+H=393.1, Rₜ: 4.0 min.

### Step 5: Preparation of 1⁵-(4-methylpiperazine-1-carbonyl)-1¹H-2,6-diaza-1(2,1)-benzo[d]imidazola-4(1,3)-benzenacycloundecaphane-3,5-dione

A solution of 3,5-dioxo-1¹H-2,6-diaza-1(2,1)-benzo[d]imidazola-4(1,3)-benzenacycloundecaphane-1⁵-carboxylic acid (20 mg, 0.050 mmol), N-methylpiperazine (7.5 mg, 0.075 mmol), HATU (19 mg, 0.050 mmol) and TEA (21 μl, 0.15 mmol) in 0.5 ml MeCN was stirred over night at 50° C. The reaction mixture was diluted with water and methanol, filtered and purified with reverse phase chromatography (Gemini NX-C18, 21*150 mm, 50 mM NH₄OH (aq)/acetonitrile, gradient over to 12 minutes, 25 ml/min). The pure fractions were pooled and concentrated to give the product as a white solid (1.5 mg, 6% yield). ¹H NMR (DMSO-d₆) δ: 12.57 (s, 1H), 9.03 (s, 1H), 8.11 (d, 1H), 7.89 (m, 1H), 7.75 (d, 1H), 7.58 (m, 1H), 7.55 (d, 1H), 7.51 (s, 1H), 7.28 (d, 1H), 4.29 (m, 2H), 3.48 (bs, 4H), 3.23-3.15 (m, 2H), 2.33 (bs, 2H), 2.20 (s, 3H), 2.04 (bs, 2H), 1.83 (bs, 2H), 1.43 (bs, 2H); LC/MS: M+H=475.2

### Example 12: 1⁵-(4-(oxetan-3-yl)piperazine-1-carbonyl)-1¹H-2,6-diaza-1(2,1)-benzo[d]imidazola-4(1,3)-benzenacycloundecaphane-3,5-dione

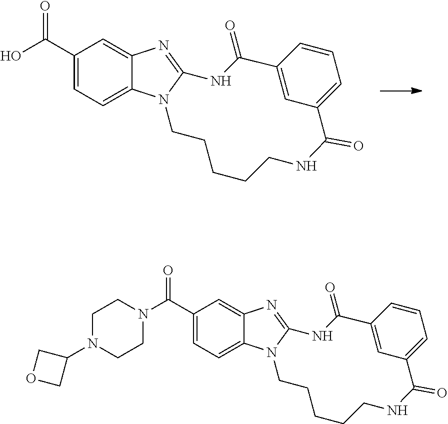 [SMILES: O=C(O)c1ccc2c(c1)nc1n2CCCCCNC(=O)c2cccc(c2)C(=O)N1 ; O=C1NCCCCCn2c(nc3cc(C(=O)N4CCN(C5COC5)CC4)ccc32)NC(=O)c2cccc1c2]

A solution of 3,5-dioxo-1¹H-2,6-diaza-1(2,1)-benzo[d]imidazola-4(1,3)-benzenacycloundecaphane-1⁵-carboxylic acid (20 mg, 0.050 mmol), 1-(oxetan-3-yl)piperazine (11 mg, 0.075 mmol), HATU (19 mg, 0.050 mmol) and TEA (21 μl, 0.15 mmol) in 0.5 ml MeCN was stirred over night at 50° C. The reaction mixture was diluted with water and methanol, filtered and purified with reverse phase chromatography (Gemini NX-C18, 21*150 mm, 50 mM NH₄OH (aq)/acetonitrile, gradient over to 12 minutes, 25 ml/min). The pure fractions were pooled and concentrated to give the product as a white solid (2.1 mg, 8% yield). ¹H NMR (DMSO-d₆) δ: 9.03 (s, 1H), 8.11 (d, 1H), 7.89 (m, 1H), 7.75 (d, 1H), 7.62-7.53 (m, 2H), 7.51 (s, 1H), 7.29 (d, 1H), 4.54 (m, 2H), 4.45 (m, 2H), 4.29 (m, 2H), 3.52 (bs, 4H), 3.45 (m, 1H), 3.23-3.14 (m, 2H), 2.30 (bs, 4H), 2.04 (bs, 2H), 1.83 (bs, 2H), 1.43 (bs, 2H); LC/MS: M+H=517.2.

### Example 13: tert-butyl 4-(3,5-dioxo-1¹H-2,6-diaza-1(2,1)-benzo[d]imidazola-4(1,3)-benzenacycloundecaphane-1⁵-carbonyl)piperazine-1-carboxylate

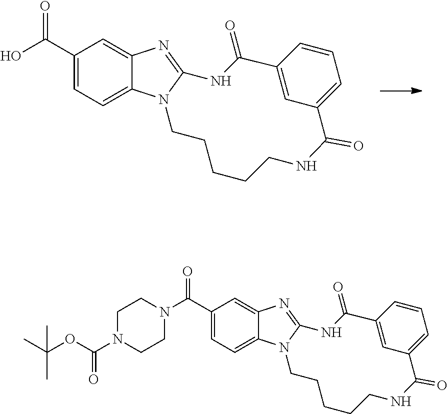 [SMILES: O=C(O)c1ccc2c(c1)nc1n2CCCCCNC(=O)c2cccc(c2)C(=O)N1 ; CC(C)(C)OC(=O)N1CCN(C(=O)c2ccc3c(c2)nc2n3CCCCCNC(=O)c3cccc(c3)C(=O)N2)CC1]

A solution of 3,5-dioxo-1¹H-2,6-diaza-1(2,1-benzo[d]imidazola-4(1,3)-benzenacycloundecaphane-1⁵-carboxylic acid (20 mg, 0.050 mmol), tert-butyl piperazine-1-carboxylate (14 mg, 0.075 mmol), HATU (19 mg, 0.050 mmol) and TEA (21 μl, 0.15 mmol) in 0.5 ml MeCN was stirred over night at 50° C. The reaction mixture was diluted with water and methanol, filtered and purified with reverse phase chromatography (Gemini NX-C18, 21*150 mm, 50 mM NH₄OH (aq)/acetonitrile, gradient over to 12 minutes, 25 ml/min). The pure fractions were pooled and concentrated to give the product as a white solid (1 mg, 4% yield). ¹H NMR (DMSO-d₆) δ: 12.62 (s, 1H), 9.03 (s, 1H), 8.11 (d, 1H), 7.88 (m, 1H), 7.75 (d, 1H), 7.62-7.50 (m, 3H), 7.30 (d, 1H), 4.29 (m, 2H), 3.50 (bs, 4H), 3.38 (bs, 4H), 3.23-3.15 (m, 2H), 2.03 (bs, 2H), 1.83 (bs, 2H), 1.41 (s, 1¹H); LC/MS: M+H=561.3

### Example 14: tert-butyl 4-(3,5-dioxo-1¹H-2,6-diaza-1(2,1)-benzo[d]imidazola-4(1,3)-benzenacycloundecaphane-1⁷-carbonyl)piperazine-1-carboxylate

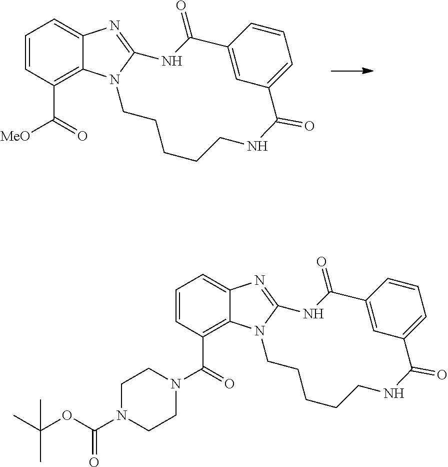 [SMILES: COC(=O)c1cccc2nc3n(c12)CCCCCNC(=O)c1cccc(c1)C(=O)N3 ; CC(C)(C)OC(=O)N1CCN(C(=O)c2cccc3nc4n(c23)CCCCCNC(=O)c2cccc(c2)C(=O)N4)CC1]

NaOH (20 mg, 0.5 mmol) was added to a solution of methyl 3,5-dioxo-1¹H-2,6-diaza-1(2,1)-benzo[d]imidazola-4(1,3)-benzenacycloundecaphane-1⁷-carboxylate (41 mg, 0.100 mmol) in 4 ml dioxane/water (1:1). The reaction mixture was stirred over night at room temperature and then acidified with 1 ml 1M HCl. The reaction mixture was concentrated under reduced pressure. The residue was dissolved in MeCN and toluene and repeatedly concentrated to dryness under reduced pressure. The residue was dissolved in 5 ml MeCN and tert-butyl piperazine-1-carboxylate (28 mg, 0.150 mmol), HATU (38 mg, 0.100 mmol) and TEA (42 μl, 0.30 mmol) was added. The resulting solution was stirred over night at 50° C. The reaction mixture was diluted with water and methanol, filtered and purified with reverse phase chromatography (Gemini NX-C18, 21*150 mm, 50 mM NH₄OH (aq)/acetonitrile, gradient over to 12 minutes, 25 ml/min). The pure fractions were pooled and concentrated to give the product as a white solid (15 mg, 27% yield). ¹H NMR (DMSO-d₆) δ: 12.72 (s, 1H), 8.97 (s, 1H), 8.11 (d, 1H), 7.86 (m, 1H), 7.76 (d, 1H), 7.61-7.54 (m, 2H), 7.25 (m, 1H), 7.13 (d, 1H), 4.29-4.20 (m, 1H), 4.13-4.04 (m, 1H), 3.76-3.67 (m, 2H), 3.53-3.40 (m, 2H), 3.30-3.22 (m, 5H), 3.19-3.09 (m, 1H), 2.03-1.93 (m, 2H), 1.80-1.70 (m, 2H), 1.41 (s, 1¹H); LC/MS: M+H=561.3.

### Example 15: 1′-methyl-1⁷-(piperazine-1-carbonyl)-1¹H-2,6-diaza-1(2,1)-benzo[d]imidazola-4(1,3)-benzenacycloundecaphane-3,5-dione

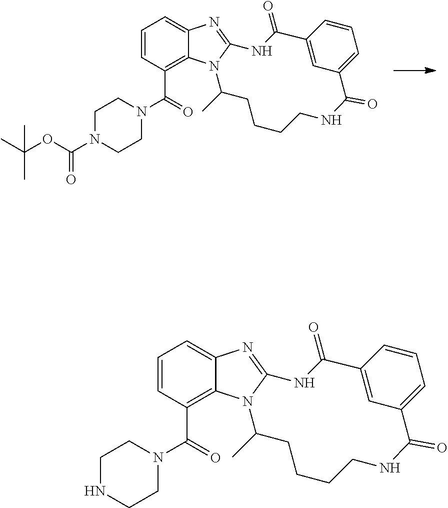 [SMILES: CC1CCCCNC(=O)c2cccc(c2)C(=O)Nc2nc3cccc(C(=O)N4CCN(C(=O)OC(C)(C)C)CC4)c3n21 ; CC1CCCCNC(=O)c2cccc(c2)C(=O)Nc2nc3cccc(C(=O)N4CCNCC4)c3n21]

To a stirred solution of tert-butyl 4-(11-methyl-3,5-dioxo-1¹H-2,6-diaza-1(2,1)-benzo[d]imidazola-4(1,3)-benzenacycloundecaphane-1⁷-carbonyl)piperazine-1-carboxylate (13 mg, 0.023 mmol) in 0.3 ml CH₂Cl₂ was added 0.2 ml TFA. The reaction mixture was stirred for 3 hours at room temperature and concentrated to dryness. The reaction mixture was diluted with water and methanol, filtered and purified with reverse phase chromatography (Gemini NX-C18, 21*150 mm, 50 mM NH₄OH (aq)/acetonitrile, gradient over to 12 minutes, 25 ml/min). The pure fractions were pooled and concentrated to give the product as a white solid (6 mg, 56% yield). ¹H NMR (DMSO-d₆) δ: 8.76 (s, 1H), 8.11 (d, 1H), 7.80 (d1H), 7.74 (d, 1H), 7.59 (tm, 1H), 7.54 (d, 1H), 7.23 (m, 1H), 7.04 (d, 1H), 4.58 (bs, 1H), 2.68 (m, 2H); LC/MS: M+H=475.2

### Example 16: 1⁵-(piperazine-1-carbonyl)-1¹H-2,6-diaza-1(2,1)-benzo[d]imidazola-4(1,3)-benzenacycloundecaphane-3,5-dione

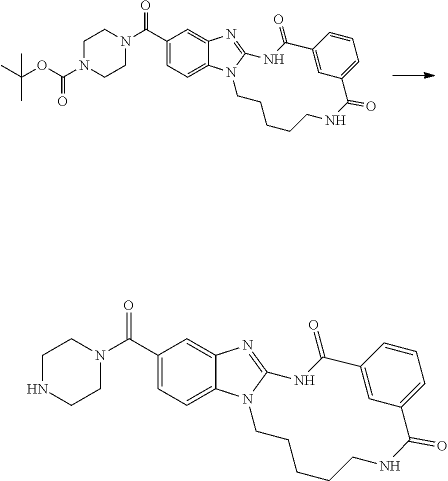 [SMILES: CC(C)(C)OC(=O)N1CCN(C(=O)c2ccc3c(c2)nc2n3CCCCCNC(=O)c3cccc(c3)C(=O)N2)CC1 ; O=C1NCCCCCn2c(nc3cc(C(=O)N4CCNCC4)ccc32)NC(=O)c2cccc1c2]

To a stirred solution of tert-butyl 4-(3,5-dioxo-1¹H-2,6-diaza-1(2,1)-benzo[d]imidazola-4(1,3)-benzenacycloundecaphane-1⁵-carbonyl)piperazine-1-carboxylate (3 mg, 0.005 mmol) in 0.3 ml CH₂Cl₂ was added 0.2 ml TFA. The reaction mixture was stirred for 3 hours at room temperature and concentrated to dryness. The reaction mixture was diluted with water and methanol, filtered and purified with reverse phase chromatography (Gemini NX-C18, 21*150 mm, 50 mM NH₄OH (aq)/acetonitrile, gradient over to 12 minutes, 25 ml/min). The pure fractions were pooled and concentrated to give the product as a white solid (2 mg, 81% yield). ¹H NMR (DMSO-d₆) δ: 9.03 (s, 1H), 8.11 (d, 1H), 7.88 (m, 1H), 7.75 (d, 1H), 7.58 (m, 1H), 7.54 (d, 1H), 7.49 (s, 1H), 7.27 (d1H), 4.29 (m, 2H), 3.42 (s, 4H), 3.23-3.16 (m, 2H), 2.69 (s, 4H), 2.03 (s, 2H), 1.83 (s, 2H), 1.43 (s, 2H). LC/MS: M+H=461.2

### Example 17: 1⁷-(piperazine-1-carbonyl)-1¹H-2,6-diaza-1(2,1)-benzo[d]imidazola-4(1,3)-benzenacycloundecaphane-3,5-dione

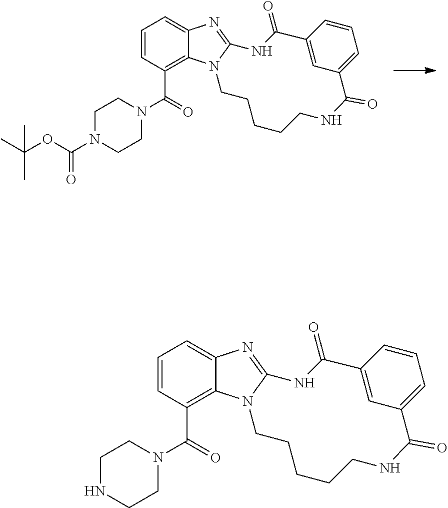 [SMILES: CC(C)(C)OC(=O)N1CCN(C(=O)c2cccc3nc4n(c23)CCCCCNC(=O)c2cccc(c2)C(=O)N4)CC1 ; O=C1NCCCCCn2c(nc3cccc(C(=O)N4CCNCC4)c32)NC(=O)c2cccc1c2]

To a stirred solution of tert-butyl 4-(3,5-dioxo-1¹H-2,6-diaza-1(2,1)-benzo[d]imidazola-4(1,3)-benzenacycloundecaphane-1⁷-carbonyl)piperazine-1-carboxylate (15 mg, 0.027 mmol) in 0.5 ml CH₂Cl₂ was added 0.2 ml TFA. The reaction mixture was stirred for 3 hours at room temperature and concentrated to dryness. The reaction mixture was diluted with water and methanol, filtered and purified with reverse phase chromatography (Gemini NX-C18, 21*150 mm, 50 mM NH₄OH (aq)/acetonitrile, gradient over to 12 minutes, 25 ml/min). The pure fractions were pooled and concentrated to give the product as a white solid (10 mg, 81% yield). ¹H NMR (DMSO-d₆) δ: 8.96 (s, 1H), 8.11 (d, 1H), 7.86 (m, 1H), 7.75 (d, 1H), 7.58 (m, 1H), 7.54 (d, 1H), 7.24 (m, 1H), 7.08 (d, 1H), 4.28-4.07 (m, 2H), 2.85-2.53 (m, 4H), 1.99 (bs, 2H), 1.76 (bs, 2H), 1.40 (bs, 2H). LC/MS: M+H=461.2

### Example 18: (E)-3,5-dioxo-1²,1³-dihydro-1¹H-2,6-diaza-1(2,1)-benzo[d]imidazola-4(1,3)-benzenacycloundecaphane-1⁷-carboxylic acid

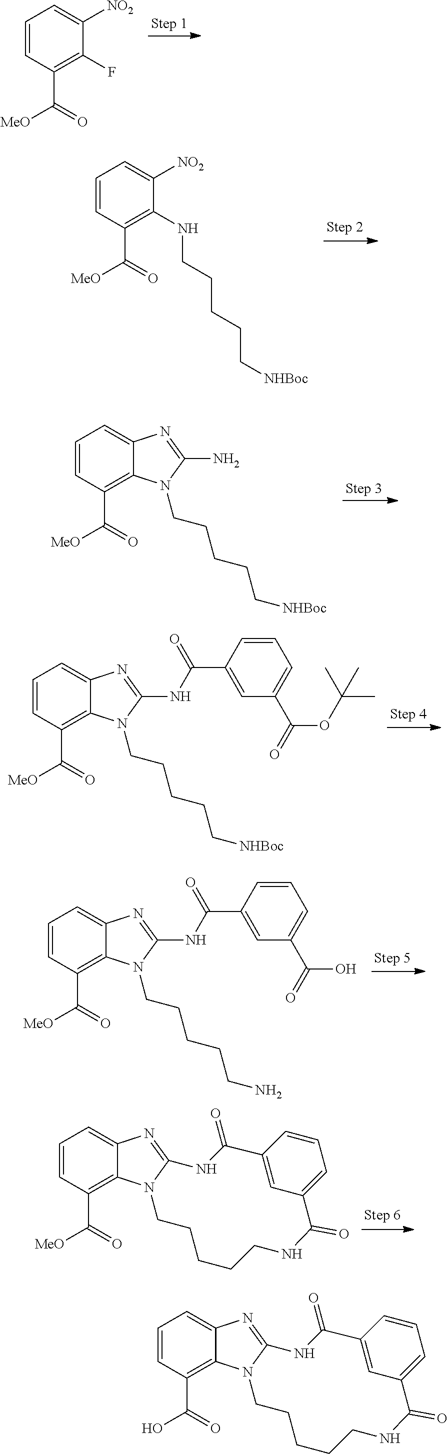

### Step 1: Preparation of methyl 2-((5-((tert-butoxycarbonyl)amino)pentyl)amino)-3-nitrobenzoate

A mixture of methyl 2-fluoro-3-nitrobenzoate (1.0 g, 5.02 mmol), N-boc-1,5-diaminopentane (1.22 g, 6.03 mmol) and K₂CO₃ (2.08 g, 15.1 mmol) in acetonitrile (20 ml) was stirred at 80° C. overnight. The solvent was removed under reduced pressure and the residue partitioned between water and ethyl acetate. The organic phase was washed with 0.1 M NaOH, 0.1 M HCl, sat. NaHCO₃ and brine, dried over MgSO₄, filtered and concentrated. Crude methyl 2-((5-((tert-butoxycarbonyl)amino)pentyl)amino)-3-nitrobenzoate was used in the next step without further purification.

¹H NMR (DMSO-d₆) δ: (m, 1H), 8.05 (m, 1H), 8.04-8.02 (m, 1H), 6.82-6.73 (m, 2H), 3.86 (s, 3H), 2.90-2.81 (m, 4H), 1.56 (m, 2H), 1.39-1.31 (m, 1¹H), 1.30-1.22 (m, 2H); LC/MS: M+H=382.1.

### Step 2: Preparation of methyl 2-amino-1-(5-((tert-butoxycarbonyl)amino)pentyl)-1H-benzo[d]imidazole-7-carboxylate

5% Pd/C (265 mg, 249 μmol) was added to a solution of methyl 2-((5-((tert-butoxycarbonyl)amino)pentyl)amino)-3-nitrobenzoate (1.90 g, 4.98 mmol) in methanol (20 ml). The reaction flask was evacuated and flushed with hydrogen twice. The mixture was stirred under hydrogen for 2 hours. The mixture was filtered through celite and cyanogen bromide (686 mg, 6.48 mmol) was added to the solution and the mixture was stirred overnight at 40° C. The reaction mixture was concentrated under reduced pressure and the residue partitioned between sat. NaHCO₃ and ethyl acetate. The organic phase was washed with sat. NaHCO₃ and brine, dried over MgSO₄, filtered and concentrated. Crude methyl 2-amino-1-(5-((tert-butoxycarbonyl)amino)pentyl)-1H-benzo[d]imidazole-7-carboxylate was used directly in the next step without further purification.

¹H NMR (DMSO-d₆) δ: 7.33 (m, 1H), 7.24 (m, 1H), 7.00 (m, 1H), 6.74 (m, 1H), 6.57 (s, 2H), 4.16-4.08 (m, 2H), 3.87 (s, 3H), 2.85 (m, 2H), 1.43 (p, J=7.7 Hz, 2H), 1.39-1.29 (m, 1¹H), 1.15 (m, 2H); LC/MS: M+H=377.2.

### Step 3: Preparation of methyl 1-(5-((tert-butoxycarbonyl)amino)pentyl)-2-(3-(tert-butoxycarbonyl)benzamido)-1H-benzo[d]imidazole-7-carboxylate

A solution of methyl 2-amino-1-(5-((tert-butoxycarbonyl)amino)pentyl)-1H-benzo[d]imidazole-7-carboxylate (1.0 g, 2.66 mmol), 3-(tert-butoxycarbonyl)benzoic acid (590 mg, 2.66 mmol), HATU (1.01 g, 2.66 mmol) and TEA (1.11 ml, 7.97 mmol) in THF (100 ml) was stirred at 50° C. overnight. The solvent was removed under reduced pressure and the residue partitioned between water and ethyl acetate.

The organic phase was washed with 0.1 M NaOH, 0.1 M HCl, sat. NaHCO₃ and brine, dried over MgSO₄, filtered and concentrated. The residue was dissolved in acetonitrile and precipitated by the addition of water. The solid was collected by filtration and washed with acetonitrile/water. Methyl 1-(5-((tert-butoxycarbonyl)amino)pentyl)-2-(3-(tert-butoxycarbonyl)benzamido)-1H-benzo[d]imidazole-7-carboxylate was used without further purification.

¹H NMR (DMSO-d₆) δ: 13.11 (s, 1H), 8.79 (s, 1H), 8.45 (d, 1H), 8.05 (d, 1H), 7.80 (m, 1H), 7.64-7.58 (m, 2H), 7.32 (m, 1H), 6.70 (m, 1H), 4.48 (m, 2H), 3.95 (s, 3H), 2.88 (m, 2H), 1.64 (m, 2H), 1.59 (s, 9H), 1.43 (m, 2H), 1.36-1.25 (m, 1¹H); LC/MS: M+H=581.3.

### Step 4: Preparation of 1-(5-aminopentyl)-2-(3-carboxybenzamido)-1H-benzo[d]imidazole-7-carboxylic acid

Methyl 1-(5-((tert-butoxycarbonyl)amino)pentyl)-2-(3-(tert-butoxycarbonyl)benzamido)-1H-benzo[d]imidazole-7-carboxylate (1.20 g, 2.07 mmol) was dissolved in dioxane (10 ml) and water (5 ml) and conc. HCl (5 ml) was added. The reaction mixture was stirred over night at room temperature. The solution was concentrated to dryness and the residue refluxed in diethyl ether/methanol until a fine precipitate formed. The solid was collected by filtration and dried under vacuum. 1-(5-aminopentyl)-2-(3-carboxybenzamido)-1H-benzo[d]imidazole-7-carboxylic acid was used without further purification.

¹H NMR (DMSO-d₆) δ: 8.81 (m, 1H), 8.46 (m, 1H), 8.10 (m, 1H), 7.89 (br. s, 3H), 7.82 (m, 1H), 7.67-7.60 (m, 2H), 7.33 (m, 1H), 4.50-4.44 (m, 2H), 3.96 (s, 3H), 2.76 (m, 2H), 1.70 (m, 2H), 1.62 (m, 2H), 1.37 (m, 2H); LC/MS: M+H=425.2.

### Step 5: Preparation of methyl 3,5-dioxo-1¹H-2,6-diaza-1(2,1)-benzo[d]imidazola-4(1,3)-benzenacycloundecaphane-1⁷-carboxylate

A slurry of 1-(5-aminopentyl)-2-(3-carboxybenzamido)-1H-benzo[d]imidazole-7-carboxylic acid (220 mg, 477 μmol) and TEA (333 μl, 2.39 mmol) in THF (200 ml) was added dropwise over 1 hour to a solution of Pybrop (334 mg, 716 μmol) in THF (200 ml). The reaction mixture was stirred at room temperature overnight and at 60° C. for 4 hours. The reaction mixture was filtered and concentrated under reduced pressure. The residue was slurried in water and concentrated. The remaining solid was slurried in methanol and collected by filtration. The solid was washed with methanol and water/methanol mixture. Methyl 3,5-dioxo-1¹H-2,6-diaza-1(2,1)-benzo[d]imidazola-4(1,3)-benzenacycloundecaphane-1⁷-carboxylate (140 mg, 72%) was used without further purification in the next step.

¹H NMR (DMSO-d₆) δ: 12.84 (s, 1H), 8.96 (s, 1H), 8.11 (d, 1H), 7.86 (m, 1H), 7.78 (d, 1H), 7.73 (d, 1H), 7.62-7.56 (m, 2H), 7.29 (m, 1H), 4.35 (m, 2H), 3.92 (s, 3H), 3.27 (br. s, 2H), 1.99 (br. s, 2H), 1.89 (br. s, 2H), 1.46 (br. s, 2H); LC/MS: M+H=407.2.

### Step 6: Preparation of 3,5-dioxo-1¹H-2,6-diaza-1(2,1)-benzo[d]imidazola-4(1,3)-benzenacycloundecaphane-1⁷-carboxylic acid

A solution of methyl 3,5-dioxo-1¹H-2,6-diaza-1(2,1)-benzo[d]imidazola-4(1,3)-benzenacycloundecaphane-1⁷-carboxylate (165 mg, 406 μmol) and NaOH (114 mg, 2.84 mmol) in dioxane (5 ml) and water (2 ml) was stirred at 40° C. for 1 day. The reaction mixture was diluted with water acidified with 4 ml of 1 M HCl. Cloudy, gel-like precipitate. The gel was centrifuged and the solid washed with water/acetonitrile twice. 20 mg of the residue dissolved in water (2 ml) with a drop of 28% ammonia. The compound was purified with reverse phase chromatography (Gemini NX-C18, 21*150 mm, water (50 mM NH₄OH)/acetonitrile, gradient over 12 minutes, 25 ml/min) to give 3,5-dioxo-1¹H-2,6-diaza-1(2,1)-benzo[d]imidazola-4(1,3)-benzenacycloundecaphane-1⁷-carboxylic acid.

¹H NMR (DMSO-d₆) δ: 8.98 (s, 1H), 8.10 (d, 1H), 7.85 (m, 1H), 7.76 (d, 1H), 7.61-7.53 (m, 2H), 7.41 (d, 1H), 7.17 (m, 1H), 4.50 (m, 2H), 3.28-3.21 (m, 2H), 1.98 (s, 2H), 1.86 (s, 2H), 1.41 (s, 2H); LC/MS: M+H=393.1.

### General Method A (Used in the Synthesis of Example Compounds 19 to 26):

A solution of corresponding acid (1 eq), amine (1.5 eq), HATU and triethylamine in MeCN were stirred overnight at 50° C. The reaction mixture was diluted with water and methanol and purified with reverse phase chromatography (50 mM NH₄OH aqueous solution/acetonitrile). The pure fractions were pooled and concentrated to give the desired product.

### Example 19: (E)-1⁷-(4-(oxetan-3-yl)piperazine-1-carbonyl)-1²,1³-dihydro-1¹H-2,6-diaza-1(2,1)-benzo[d]imidazola-4(1,3)-benzenacycloundecaphane-3,5-dione

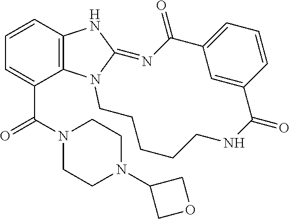 [SMILES: O=C1/N=C2\Nc3cccc(C(=O)N4CCN(C5COC5)CC4)c3N2CCCCCNC(=O)c2cccc1c2]

Prepared according to general method A starting from (E)-3,5-dioxo-1²,1³-dihydro-1¹H-2,6-diaza-1(2,1)-benzo[d]imidazola-4(1,3)-benzenacycloundecaphane-1⁷-carboxylic acid and 1-(oxetan-3-yl)piperazine.

1H NMR (DMSO-d6) δ: 12.70 (s, 1H), 8.97 (s, 1H), 8.11 (d, 1H), 7.87 (m, 1H), 7.76 (d, 1H), 7.61-7.53 (m, 2H), 7.24 (m, 1H), 7.09 (d, 1H), 4.54 (m, 2H), 4.43 (m, 2H), 4.27-4.08 (m, 2H), 3.88-3.81 (m, 1H), 3.72-3.64 (m, 1H), 3.45 (m, 1H), 3.27-3.11 (m, 2H), 2.33-2.25 (m, 2H), 2.14-2.08 (m, 1H), 1.99 (br. s, 2H), 1.75 (br. s, 2H), 1.41 (br. s, 2H); LC/MS: M+H=517.3.

### Example 20: (R,E)-1⁷-(2,4-dimethylpiperazine-1-carbonyl)-1²,1³-dihydro-1¹H-2,6-diaza-1(2,1)-benzo[d]imidazola-4(1,3)-benzenacycloundecaphane-3,5-dione

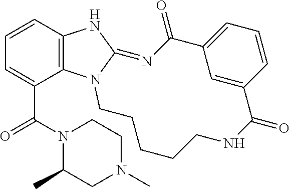 [SMILES: C[C@@H]1CN(C)CCN1C(=O)c1cccc2c1N1CCCCCNC(=O)c3cccc(c3)C(=O)/N=C/1N2]

Prepared according to general method A starting from (E)-3,5-dioxo-1²,1³-dihydro-1¹H-2,6-diaza-1(2,1)-benzo[d]imidazola-4(1,3)-benzenacycloundecaphane-1⁷-carboxylic acid and (R)-1,3-dimethylpiperazine.

1H NMR (DMSO-d6) δ: 12.69 (s, 1H), 8.96 (s, 1H), 8.11 (d, 1H), 7.88-7.82 (m, 1H), 7.75 (d, 1H), 7.60-7.51 (m, 2H), 7.23 (m, 1H), 7.13-6.95 (m, 1H); LC/MS: M+H=489.3.

### Example 21: (S,E)-1⁷-(3-(dimethylamino)pyrrolidine-1-carbonyl)-1²,1³-dihydro-1¹H-2,6-diaza-1(2,1)-benzo[d]imidazola-4(1,3)-benzenacycloundecaphane-3,5-dione

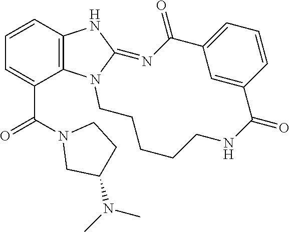 [SMILES: CN(C)[C@H]1CCN(C(=O)c2cccc3c2N2CCCCCNC(=O)c4cccc(c4)C(=O)/N=C/2N3)C1]

Prepared according to general method A starting from (E)-3,5-dioxo-1² 1³-dihydro-1¹H-2,6-diaza-1(2,1)-benzo[d]imidazola-4(1,3)-benzenacycloundecaphane-1⁷-carboxylic acid and (3S)-(−)-3-(dimethylamino)pyrrolidine.

1H NMR (DMSO-d6) δ 12.41 (s, 1H), 8.98 (s, 1H), 8.11 (d, 1H), 7.86 (m, 1H), 7.76 (d, 1H), 7.61-7.52 (m, 2H), 7.24 (m, 1H), 7.15 (m, 1H), 4.18 (s, 2H), 3.85-3.70 (m, 1H), 2.78-2.64 (m, 1H), 2.19 (s, 3H), 2.05 (s, 3H), 1.99 (s, 2H), 1.83-1.75 (m, 1H), 1.73 (s, 2H), 1.41 (s, 2H); LC/MS: M+H=489.3.

### Example 22: (E)-1⁷-((1S,4S)-5-methyl-2,5-diazabicyclo[2.2.1]heptane-2-carbonyl)-1²,1³-dihydro-1¹H-2,6-diaza-1(2,1)-benzo[d]imidazola-4(1,3)-benzenacycloundecaphane-3,5-dione

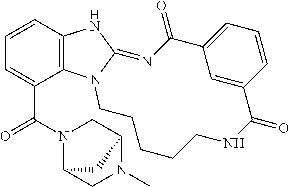 [SMILES: CN1C[C@@H]2C[C@H]1CN2C(=O)c1cccc2c1N1CCCCCNC(=O)c3cccc(c3)C(=O)/N=C/1N2]

Prepared according to general method A starting from (E)-3,5-dioxo-1²,1³-dihydro-1¹H-2,6-diaza-1(2,1)-benzo[d]imidazola-4(1,3)-benzenacycloundecaphane-1⁷-carboxylic acid and (1S,4S)-5-methyl-2,5-diazabicyclo[2.2.1]heptane.

1H NMR (DMSO-d6) δ 12.42 (s, 1H), 8.97 (s, 1H), 8.11 (d, 1H), 7.89-7.83 (m, 1H), 7.75 (d, 1H), 7.61-7.53 (m, 2H), 7.29-7.19 (m, 1H), 7.15 (d, 1H); LC/MS: M+H=487.2.

### Example 23: (R,E)-1⁷-(3-(dimethylamino)pyrrolidine-1-carbonyl)-1²,1³-dihydro-1¹H-2,6-diaza-1(2,1)-benzo[d]imidazola-4(1,3)-benzenacycloundecaphane-3,5-dione

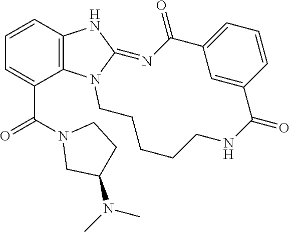 [SMILES: CN(C)[C@@H]1CCN(C(=O)c2cccc3c2N2CCCCCNC(=O)c4cccc(c4)C(=O)/N=C/2N3)C1]

Prepared according to general method A starting from (E)-3,5-dioxo-1²,1³-dihydro-1¹H-2,6-diaza-1(2,1)-benzo[d]imidazola-4(1,3)-benzenacycloundecaphane-1⁷-carboxylic acid and (3R)-(−)-3-(dimethylamino)pyrrolidine.

1H NMR (DMSO-d6) δ 8.97 (s, 1H), 8.11 (d, 1H), 7.88 (m, 1H), 7.75 (d, 1H), 7.61-7.53 (m, 2H), 7.24 (m, 1H), 7.15 (m, 1H), 4.17 (s, 2H), 3.84-3.70 (m, 1H), 2.19 (s, 3H), 2.05 (s, 3H), 1.73 (s, 2H), 1.41 (s, 2H); LC/MS: M+H=489.3.

### Example 24: (S,E)-1⁷-(2,4-dimethylpiperazine-1-carbonyl)-1²,1³-dihydro-1¹H-2,6-diaza-1(2,1)-benzo[d]imidazola-4(1,3)-benzenacycloundecaphane-3,5-dione

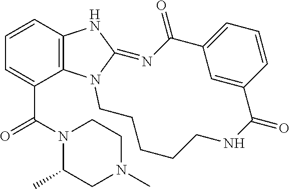 [SMILES: C[C@H]1CN(C)CCN1C(=O)c1cccc2c1N1CCCCCNC(=O)c3cccc(c3)C(=O)/N=C/1N2]

Prepared according to general method A starting from (E)-3,5-dioxo-1²,1³-dihydro-1¹H-2,6-diaza-1(2,1)-benzo[d]imidazola-4(1,3)-benzenacycloundecaphane-1⁷-carboxylic acid and (S)-1,3-dimethyl-piperazine.

1H NMR (DMSO-d6) δ 12.77 (s, 1H), 9.76 (s, 1H), 8.98 (d, 1H), 8.11 (d, 1H), 7.88 (m, 1H), 7.77 (d, 1H), 7.63-7.55 (m, 2H), 7.32-7.04 (m, 2H); LC/MS: M+H=489.3.

### Example 25: (E)-1⁷-((1R,4R)-5-methyl-2,5-diazabicyclo[2.2.1]heptane-2-carbonyl)-1²,1³-dihydro-1¹H-2,6-diaza-1(2,1)-benzo[d]imidazola-4(1,3)-benzenacycloundecaphane-3,5-dione

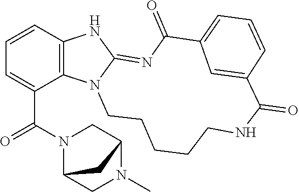 [SMILES: CN1C[C@H]2C[C@@H]1CN2C(=O)c1cccc2c1N1CCCCCNC(=O)c3cccc(c3)C(=O)/N=C/1N2]

Prepared according to general method A starting from (E)-3,5-dioxo-1²,1³-dihydro-1¹H-2,6-diaza-1(2,1)-benzo[d]imidazola-4(1,3)-benzenacycloundecaphane-1⁷-carboxylic acid and (1R,4R)-5-methyl-2,5-diazabicyclo-[2.2.1]heptane.

1H NMR (DMSO-d6) δ 12.76 (s, 1H), 8.98 (s, 1H), 8.12 (d, 1H), 7.87 (m, 1H), 7.77 (d, 1H), 7.60 (m, 2H), 7.33-7.16 (m, 2H), 1.99 (s, 2H), 1.77 (s, 2H), 1.43 (s, 2H); LC/MS: M+H=487.3.

### Example 26: (E)-1⁷-(4-methyl-1,4-diazepane-1-carbonyl)-1²,1³-dihydro-1¹H-2,6-diaza-1(2,1)-benzo[d]imidazola-4(1,3)-benzenacycloundecaphane-3,5-dione

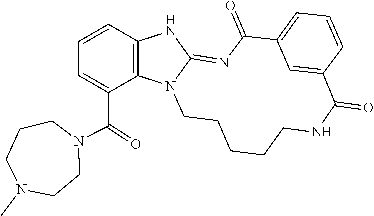 [SMILES: CN1CCCN(C(=O)c2cccc3c2N2CCCCCNC(=O)c4cccc(c4)C(=O)/N=C/2N3)CC1]

Prepared according to general method A starting from (E)-3,5-dioxo-1²,1³-dihydro-1¹H-2,6-diaza-1(2,1)-benzo[d]imidazola-4(1,3)-benzenacycloundecaphane-1⁷-carboxylic acid and N-methylhomopiperazine.

1H NMR (DMSO-d6) δ 12.75 (s, 1H), 9.70-9.42 (m, 1H), 8.98 (s, 1H), 8.11 (d, 1H), 7.92-7.84 (m, 1H), 7.77 (d, 1H), 7.62-7.55 (m, 2H), 7.31-7.20 (m, 2H); LC/MS: M+H=489.3.

### Example 27: (E)-N,N-dimethyl-1⁵-((4-methylpiperazin-1-yl)methyl)-3,5-dioxo-1²,1³-dihydro-1¹H-2,6-diaza-1(2,1)-benzo[d]imidazola-4(1,3)-benzenacycloundecaphane-1⁷-carboxamide

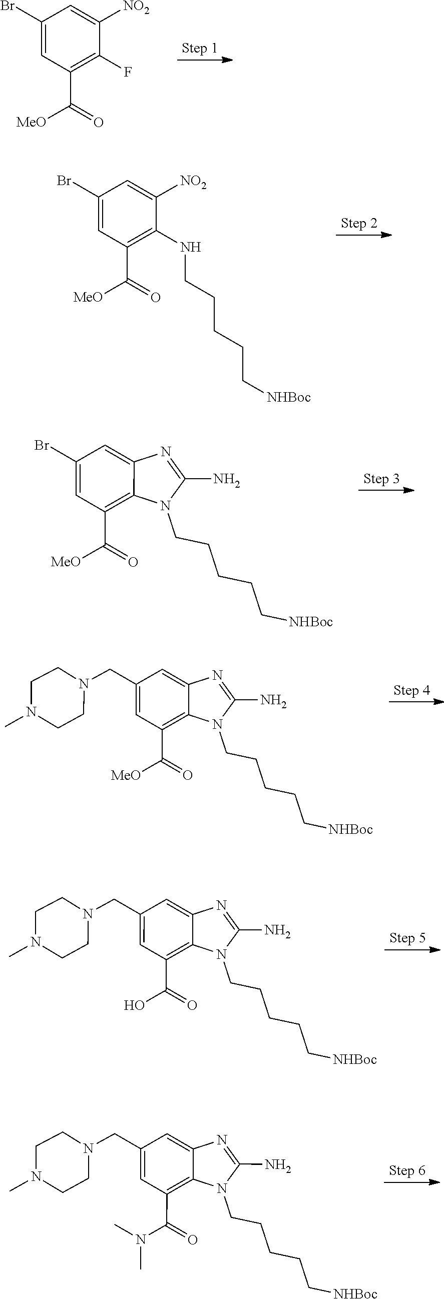 [SMILES: COC(=O)c1cc(Br)cc([N+](=O)[O-])c1F ; COC(=O)c1cc(Br)cc([N+](=O)[O-])c1NCCCCCNC(=O)OC(C)(C)C ; COC(=O)c1cc(Br)cc2nc(N)n(CCCCCNC(=O)OC(C)(C)C)c12 ; COC(=O)c1cc(CN2CCN(C)CC2)cc2nc(N)n(CCCCCNC(=O)OC(C)(C)C)c12 ; CN1CCN(Cc2cc(C(=O)O)c3c(c2)nc(N)n3CCCCCNC(=O)OC(C)(C)C)CC1 ; CN1CCN(Cc2cc(C(=O)N(C)C)c3c(c2)nc(N)n3CCCCCNC(=O)OC(C)(C)C)CC1]

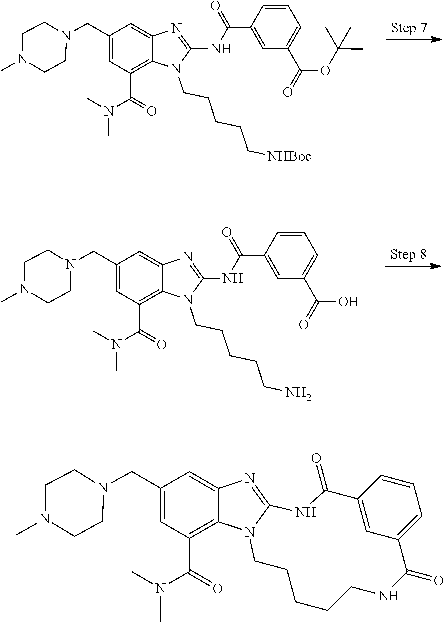

### Step 1: Preparation of methyl 5-bromo-2-((5-((tert-butoxycarbonyl)amino)pentyl)amino)-3-nitrobenzoate

A suspension of methyl 5-bromo-2-fluoro-3-nitrobenzoate (556 mg, 2.0 mmol), tert-butyl (5-aminopentyl)carbamate (809 mg, 4.0 mmol) and K₂CO₃ (829 mg, 6.0 mmol) in MeCN (10 ml) was stirred at rt overnight. The reaction mixture was concentrated and the residues were added to EtOAc (20 mL) and washed with aq. NaOH (3×20 mL, 1 M), the organic layer was dried using a phase separator and concentrated to yellow oil (1.08 g). The crude product was purified by flash chromatography on silica gel (Biotage, 0-30% EtOAc in pet. ether for 10 column) to give methyl 5-bromo-2-((5-((tert-butoxycarbonyl)amino)pentyl)amino)-3-nitrobenzoate as a yellow oil (884.9 mg, 95%).

LC/MS: M+H=462.1; Rₜ=5.85 min.

### Step 2: Preparation of methyl 2-amino-5-bromo-1-(5-((tert-butoxycarbonyl)amino)pentyl)-1H-benzo[d]imidazole-7-carboxylate

A NH₄Cl (511 mg, 9.55 mmol) was added to a solution of methyl 5-bromo-2-((5-((tert-butoxycarbonyl)amino)pentyl)amino)-3-nitrobenzoate (400 mg, 869 μmol) in EtOH/water 10:1 (22 ml) water at rt, followed by Fe powder (256 mg, 4.58 mmol). The mixture was heated to reflux (80° C.) for 2 h. The mixture was cooled to rt and filtered through celite. The filtrate was concentrated and added to EtOAc (20 mL) and washed with aq. NaOH (3×20 mL, 1 M). The combined water layers were back extracted with EtOAc (20 mL). The combined organic layers were dried and concentrated. Purification (Biotage 12 g, 1-5% MeOH in CH₂Cl₂ with 1% NH₃ (28% aq.) gave a brown oil brown oil (364 mg). The brown oil was dissolved in water (2.0 ml), MeCN (2.0 ml) and MeOH (6.0 ml) and CNBr (108 mg, 1.02 mmol) was added. The reaction mixture was stirred overnight and concentrated under reduced pressure. The residue was dissolved in acetonitrile and water (˜2 mL in total) and purified by reverse phase chromatography on a C18 flash cartridge (Biotage 12 g, 5-60% MeCN in water with 0.1% TFA) to give methyl 2-amino-5-bromo-1-(5-((tert-butoxycarbonyl)amino)pentyl)-1H-benzo[d]imidazole-7-carboxylate as a white solid (133.3 mg, 27%).

LC/MS: M+H=455.1; Rₜ=4.4 min.

### Step 3: Preparation of methyl 2-amino-1-(5-((tert-butoxycarbonyl)amino)pentyl)-5-((4-methylpiperazin-1-yl)methyl)-1H-benzo[d]imidazole-7-carboxylate

A flask was charged with Pd(OAc)₂, XPhos, Cs₂CO₃ and potassium trifluoro((4-methylpiperazin-1-yl)methyl)borate under N₂ gas and a solution of 2-amino-5-bromo-1-(5-((tert-butoxycarbonyl)amino)pentyl)-1H-benzo[d]imidazole-7-carboxylate (133 mg, 234 μmol) in THF/water at 10:1 (2.0 ml) was added at rt. The solution was heated to 80° C. for 48 h. The reaction mixture was poured into brine (25 mL) and extracted with CH₂Cl₂ (4×15 mL). The organic layers were dried and concentrated to yellow oil (155.5 mg). The residue was dissolved in DMSO/water and purified by reverse phase chromatography (C18 column, 10-45% MeCN in water containing 0.1% TFA) to give methyl 2-amino-1-(5-((tert-butoxycarbonyl as white solids (94.7 mg, 33%).

LC/MS: M+H=489.3; Rₜ=3.6 min.

### Step 4: Preparation of 2-amino-1-(5-((tert-butoxycarbonyl)amino)pentyl)-5-((4-methylpiperazin-1-yl)methyl)-1H-benzo[d]imidazole-7-carboxylic acid

5M NaOH (50 μl, 250 μmol) was added to a solution of methyl 2-amino-1-(5-((tert-butoxycarbonyl)amino)pentyl)-5-((4-methylpiperazin-1-yl)methyl)-1H-benzo[d]imidazole-7-carboxylate (47 mg, 78 μmol) in dioxane/water 1:1 (6 ml). The reaction mixture was stirred at rt for 24 h and 5 M NaOH (50 μl, 250 μmol) was added. Stirring was continued for 24 h in the solvent mixture at rt. The reaction was purified by reverse phase chromatography (C18 column, prep-HPLC, using 5-20% MeCN in water containing 0.1% ammonia) to give 2-amino-1-(5-((tert-butoxycarbonyl)amino)pentyl)-5-((4-methylpiperazin-1-yl)methyl)-1H-benzo[d]imidazole-7-carboxylic acid (13.6 mg, 36%).

LC/MS: M+H=474.0; Rₜ=2.1 min.

### Step 5: Preparation of tert-butyl (5-(2-amino-7-(dimethylcarbamoyl)-5-((4-methylpiperazin-1-yl)methyl)-1H-benzo[d]imidazol-1-yl)pentyl)carbamate

HATU (12.6 mg, 33 μmol) was added to a suspension of 2-amino-1-(5-((tert-butoxycarbonyl)amino)pentyl)-5-((4-methylpiperazin-1-yl)methyl)-1H-benzo[d]imidazole-7-carboxylic acid (13.6 mg, 28 μmol) and dimethylamine (138 μl, 277, pmol, 2M in THF) in THF (10 ml) at rt. The reaction was stirred for 2 h and then purified by reverse phase chromatography (C18 column, prep-HPLC, using 10-45% MeCN in water containing 0.1% TFA) to give tert-butyl (5-(2-amino-7-(dimethylcarbamoyl)-5-((4-methylpiperazin-1-yl)methyl)-1H-benzo[d]imidazol-1-yl)pentyl)carbamate as a clear oil (25.7 mg, 99%).

LC/MS: M+H=502.3; Rₜ=3.4 min.

### Step 6: Preparation of tert-butyl 3-((1-(5-((tert-butoxycarbonyl)amino)pentyl)-7-(dimethylcarbamoyl)-5-((4-methylpiperazin-1-yl)methyl)-1H-benzo[d]imidazol-2-yl)carbamoyl)benzoate

(3-(tert-Butoxycarbonyl)benzoic acid (12 mg, 55 μmol), HATU (21 mg, 55 μmol) and N,N-diisopropylethylamine (14 μl, 83 μmol) were dissolved in THF (5 ml) and stirred for 10 min. A solution of tert-butyl (5-(2-amino-7-(dimethylcarbamoyl)-5-((4-methylpiperazin-1-yl)methyl)-1H-benzo[d]imidazol-1-yl)pentyl)carbamate (17 mg, 27 μmol) in THF (3 ml) was added and the resulting reaction mixture was stirred overnight. The product was purified by reverse phase chromatography (C18 column, prep-HPLC, using 10-50% MeCN in water containing 0.1% TFA) to give tert-butyl 3-((1-(5-((tert-butoxycarbonyl)amino)pentyl)-7-(dimethylcarbamoyl)-5-((4-methylpiperazin-1-yl)methyl)-1H-benzo[d]imidazol-2-yl)carbamoyl)benzoate as a white solid (11.3 mg, 50%).

LC/MS: M+H=706.4; Rₜ=4.5 min.

### Step 7: Preparation of 3-((1-(5-aminopentyl)-7-(dimethylcarbamoyl)-5-((4-methylpiperazin-1-yl)methyl)-1H-benzo[d]imidazol-2-yl)carbamoyl)benzoic acid

5M HCl (1.0 ml) was added to a solution of tert-butyl 3-((1-(5-((tert-butoxycarbonyl)amino)pentyl)-7-(dimethylcarbamoyl)-5-((4-methylpiperazin-1-yl)methyl)-1H-benzo[d]imidazol-2-yl)carbamoyl)benzoate (11.3 mg, 13.8 μmol) in dioxane (1.0 ml). The solution was stirred at rt overnight. The reaction mixture was concentrated to dryness. The residue was dissolved in MeOH and concentrated to dryness from TFA. The product was used without further purification.

### Step 8: Preparation of (E)-N,N-dimethyl-1⁵-((4-methylpiperazin-1-yl)methyl)-3,5-dioxo-1²,1³-dihydro-1¹H-2,6-diaza-1(2,1)-benzo[d]imidazola-4(1,3)-benzenacycloundecaphane-1⁷-carboxamide

HATU (7.9 mg, 21 μmol) was added to a suspension of 3-((1-(5-aminopentyl)-7-(dimethylcarbamoyl)-5-((4-methylpiperazin-1-yl)methyl)-1H-benzo[d]imidazol-2-yl)carbamoyl)benzoic acid (20 mg, 13 μmol) and N,N-diisopropylethylamine (14 μl, 83 μmol) in THF (28 ml). The solution was heated at 40° C. and stirred overnight. The reaction mixture was concentrated to dryness under reduced pressure and the residue was dissolved in DMF (25 ml) and N,N-diisopropylethylamine (0.5 ml) was added. The reaction mixture was stirred at 40° C. for 2 h and then concentrated to dryness. The residue was dissolved in DMSO and a small amount of water and purified by reverse phase chromatography (C18 column, prep-HPLC, using 10-40% MeCN in water containing 0.1% TFA) to provide of (E)-N,N-dimethyl-1⁵-((4-methylpiperazin-1-yl)methyl)-3,5-dioxo-1²,1³-dihydro-1¹H-2,6-diaza-1(2,1)-benzo[d]imidazola-4(1,3)-benzenacycloundecaphane-1⁷-carboxamide (0.9 mg, 10%).

¹H NMR (Methanol-d₄) δ: 9.13 (s, 1H), 8.22 (d, 1H), 7.84 (d, 1H), 7.61 (m, 1H), 7.55 (d, J=1.2 Hz, 1H), 7.17 (d, 1H), 4.46-4.33 (m, 1H), 4.20-4.07 (m, 1H), 3.77 (s, 2H), 3.46-3.37 (m, 2H), 3.21 (s, 3H), 2.97 (s, 3H), 2.89 (s, 3H), 2.17-2.07 (m, 2H), 1.94-1.82 (m, 2H), 1.61-1.51 (m, 2H); LC/MS: M+H=532.3.

### Example 28: (E)-1⁷-(2-methoxypyridin-3-yl)-1²,1³-dihydro-1¹H-2,6-diaza-1(2,1)-benzo[d]imidazola-4(1,3)-benzenacycloundecaphane-3,5-dione

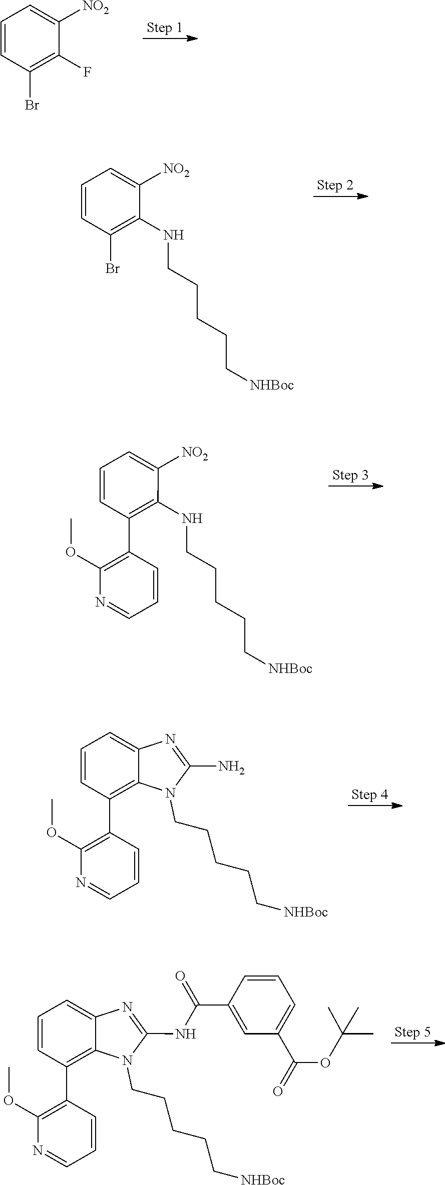 [SMILES: O=[N+]([O-])c1cccc(Br)c1F ; CC(C)(C)OC(=O)NCCCCCNc1c(Br)cccc1[N+](=O)[O-] ; COc1ncccc1-c1cccc([N+](=O)[O-])c1NCCCCCNC(=O)OC(C)(C)C ; COc1ncccc1-c1cccc2nc(N)n(CCCCCNC(=O)OC(C)(C)C)c12 ; COc1ncccc1-c1cccc2nc(NC(=O)c3cccc(C(=O)OC(C)(C)C)c3)n(CCCCCNC(=O)OC(C)(C)C)c12]

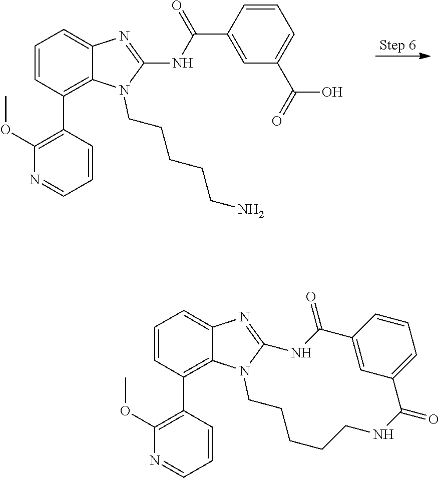 [SMILES: COc1ncccc1-c1cccc2nc(NC(=O)c3cccc(C(=O)O)c3)n(CCCCCN)c12 ; COc1ncccc1-c1cccc2nc3n(c12)CCCCCNC(=O)c1cccc(c1)C(=O)N3]

### Step 1: Preparation of tert-butyl (5-((2-bromo-6-nitrophenyl)amino)pentyl)carbamate

K₂CO₃ (2.83 g, 20.5 mmol) was added to a solution of 1-bromo-2-fluoro-3-nitrobenzene (1.50 g, 6.82 mmol) and N-boc-1,5-diaminopentane (1.66 g, 8.2 mmol) in MeCN (30 ml). The reaction mixture was stirred at 80° C. overnight. The solvent was removed under reduced pressure and the residue partitioned between water and ethyl acetate. The organic phase was washed with 0.1 M NaOH, 0.1 M HCl, sat. NaHCO₃ and brine, dried over MgSO₄, filtered and concentrated. The product of tert-butyl (5-((2-bromo-6-nitrophenyl)amino)pentyl)carbamate (2.74 g, 99%) was used without further purification.

¹H NMR (DMSO-d₆) δ: 7.83 (m, 1H), 7.82 (m, 1H), 6.82-6.77 (m, 1H), 6.74 (m, 1H), 5.95 (m, 1H), 3.04 (m, 2H), 2.86 (m, 2H), 1.49 (m, 2H), 1.39-1.29 (m, 1¹H), 1.21 (m, 2H); LC/MS: M+H-tert-Bu=347.0.

### Step 2: Preparation of tert-butyl (5-((2-(2-methoxypyridin-3-yl)-6-nitrophenyl)amino)pentyl)carbamate

A microwave vial was charged with Peppsi-iPr (13 mg, 20 μmol), 2-methoxy-3-pyridineboronic acid (151 mg, 988 μmol) and K₂CO₃ (273 mg, 1.98 mmol) and flushed with N₂. To the vial was added a solution of tert-butyl (5-((2-bromo-6-nitrophenyl)amino)pentyl)carbamate (265 mg, 659 μmol) in toluene (3.0 ml) and MeOH (1.0 ml) that had been purged with N₂. The mixture was heated in the microwave at 120° C. for 40 minutes with stirring. The reaction mixture was diluted with brine (20 mL) and extracted with EtOAc (3×20 mL). The combined organic layers were dried and concentrated to a yellow oil (350 mg). Purification by flash chromatography on silica gel (Biotage 25 g, 0-30% EtOAc in petroleum ether) to give as a tert-butyl (5-((2-bromo-6-nitrophenyl)amino)pentyl)carbamate yellow oil (265 mg, 93%).

¹H NMR (DMSO-d₆) δ: 8.24 (m, 1H), 8.05 (m, 1H), 7.67 (m, 1H), 7.46 (m, 1H), 7.32 (m, 1H), 7.09 (m, 1H), 6.82 (m, 1H), 6.70 (m, 1H), 3.84 (s, 3H), 2.78 (m, 2H), 2.55-2.51 (m, 2H), 1.35 (s, 9H), 1.30-1.13 (m, 4H), 0.96 (p, J=7.6, 7.0 Hz, 2H); LC/MS: M+H=431.2.

### Step 3: Preparation of tert-butyl (5-(2-amino-7-(2-methoxypyridin-3-yl)-1H-benzo[d]imidazol-1-yl)pentyl)carbamate

To a solution of tert-butyl (5-((2-bromo-6-nitrophenyl)amino)pentyl)carbamate (265 mg, 616 μmol) in MeOH (40 ml) under N₂, was added 10% Pd/C (32 mg, 31 μmol), and the mixture was hydrogenated at rt for 35 min. The mixture was evacuated, filtered through celite with MeOH (10 mL). The yellow brown solution was concentrated to a residue (192 mg). The residue was dissolved in water (3.0 ml) and MeOH (15.0 ml) and cyanogen bromide (78 mg, 739 μmol) was added. The reaction mixture was stirred overnight at rt. The solution was concentrated to dryness under reduced pressure. The residues were purified by flash chromatography on silica gel (Biotage 12 g 1-7% MeOH in CH₂Cl₂ with 1% NH₃ (28% aq.)) to give tert-butyl (5-(2-amino-7-(2-methoxypyridin-3-yl)-1H-benzo[d]imidazol-1-yl)pentyl)carbamate as a red oil (172.1 mg, 65%).

¹H NMR (DMSO-d₆) δ: 8.29 (m, 1H), 7.74 (m, 1H), 7.27 (d, 1H), 7.16-7.03 (m, 2H), 6.78 (d, 1H), 6.67 (m, 1H), 4.09 (m, 1H), 3.80 (s, 3H), 3.64-3.48 (m, 1H), 3.17 (d, 2H), 2.73 (m, 2H), 1.37 (s, 9H), 1.29-1.00 (m, 4H), 0.77-0.55 (m, 2H); LC/MS: M+H=426.2.

### Step 4: Preparation of tert-butyl 3-((1-(5-((tert-butoxycarbonyl)amino)pentyl)-7-(2-methoxypyridin-3-yl)-1H-benzo[d]imidazol-2-yl)carbamoyl)benzoate

A solution of tert-butyl (5-(2-amino-7-(2-methoxypyridin-3-yl)-1H-benzo[d]imidazol-1-yl)pentyl)carbamate (177 mg, 404 μmol) and N,N-diisopropylethylamine (105 μL, 0.6 mmol) in MeCN (5 mL) was added to a solution of HATU (231 mg, 607 μmol), 3-(tert-butoxycarbonyl)benzoic acid (135 mg, 607 μmol) and N,N-diisopropylethylamine (105 μL, 0.6 mmol) solution in MeCN (5 mL) at rt. The reaction mixture was stirred for 5 h and then concentrated to dryness. Purification by flash chromatography on silica gel (0-50% EtOAc in pet. Ether) gave tert-butyl 3-((1-(5-((tert-butoxycarbonyl)amino)pentyl)-7-(2-methoxypyridin-3-yl)-1H-benzo[d]imidazol-2-yl)carbamoyl)benzoate as a clear oil (215 mg, 84%).

¹H NMR (DMSO-d₆) δ: 12.97 (s, 1H), 8.76 (s, 1H), 8.42 (d, 1H), 8.33 (d, 1H), 8.02 (d, 1H), 7.84 (m, 1H), 7.65-7.54 (m, 2H), 7.28 (m, 1H), 7.17 (m, 1H), 7.02 (d, 1H), 6.64 (m, 1H), 4.06-3.96 (m, 1H), 3.82 (s, 3H), 3.63-3.49 (m, 1H), 2.74 (m, 2H), 1.56 (s, 9H), 1.34 (s, 9H), 1.30-1.01 (m, 4H), 0.93-0.65 (m, 2H); LC/MS: M+H=630.3.

### Step 5: Preparation of 3-((1-(5-aminopentyl)-7-(2-methoxypyridin-3-yl)-1H-benzo[d]imidazol-2-yl)carbamoyl)benzoic acid

5 M HCl (3 ml, 15 mmol) was added to a solution of tert-butyl 3-((1-(5-((tert-butoxycarbonyl)amino)pentyl)-7-(2-methoxypyridin-3-yl)-1H-benzo[d]imidazol-2-yl)carbamoyl)benzoate (107 mg, 170 μmol) in dioxane (10 ml) at rt. The reaction mixture was stirred for 3 days at rt. The reaction mixture was concentrated to dryness and used in the next step without purification.

LC/MS: M+H=474.2; Rₜ=4.0 min.

### Step 6: Preparation of (E)-1⁷-(2-methoxypyridin-3-yl)-1²,1³-dihydro-1¹H-2,6-diaza-1(2,1)-benzo[d]imidazola-4(1,3)-benzenacycloundecaphane-3,5-dione

HATU (97 mg, 255 μmol) was added to a solution of 3-((1-(5-aminopentyl)-7-(2-methoxypyridin-3-yl)-1H-benzo[d]imidazol-2-yl)carbamoyl)benzoic acid (100 mg, 170 μmol) and N,N-diisopropylethylamine (147 μl, 851 ummol) in DMF (350 ml). The solution was stirred overnight at rt and then concentrated to dryness. The residue was dissolved in DMSO and purified using reverse phase chromatography (30-50% MeCN in water containing 0.1% TFA) to give (E)-1⁷-(2-methoxypyridin-3-yl)-1²,1³-dihydro-1¹H-2,6-diaza-1(2,1)-benzo[d]imidazola-4(1,3)-benzenacycloundecaphane-3,5-dione (14.9 mg, 19%).

¹H NMR (DMSO-d₆) δ: 12.69 (s, 1H), 8.89 (s, 1H), 8.33 (d, 1H), 8.10 (d, 1H), 7.85-7.77 (m, 2H), 7.73 (d, 1H), 7.60-7.52 (m, 2H), 7.25 (m, 1H), 7.16 (m, 1H), 6.99 (d, 1H), 3.94-3.84 (m, 1H), 3.80 (s, 3H), 3.60-3.52 (m, 1H), 3.20-3.08 (m, 2H), 1.93-1.66 (m, 2H), 1.47-1.27 (m, 2H), 1.16-1.02 (m, 2H); LC/MS: M+H=456.2.

### Examples 29 and 30: (E)-11-methyl-1⁷-((1R,4R)-5-methyl-2,5-diazabicyclo[2.2.1]heptane-2-carbonyl)-1²,1³-dihydro-1¹H-2,6-diaza-1(2,1)-benzo[d]imidazola-4(1,3)-benzenacycloundecaphane-3,5-dione

### Example Compound 29:

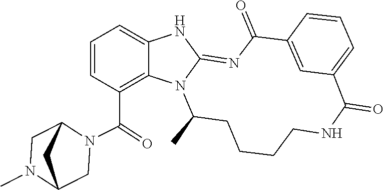 [SMILES: C[C@@H]1CCCCNC(=O)c2cccc(c2)C(=O)/N=C2\Nc3cccc(C(=O)N4C[C@H]5C[C@@H]4CN5C)c3N21]

### Example Compound 30:

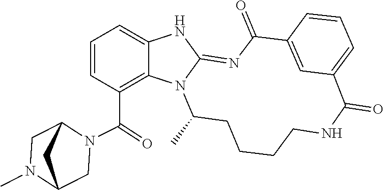 [SMILES: C[C@H]1CCCCNC(=O)c2cccc(c2)C(=O)/N=C2\Nc3cccc(C(=O)N4C[C@H]5C[C@@H]4CN5C)c3N21]

### Preparation of tert-butyl 3-((1-(5-((tert-butoxycarbonyl)amino)pentyl)-7-(2-methoxypyridin-3-yl)-1H-benzo[d]imidazol-2-yl)carbamoyl)benzoate

HATU (70 mg, 184 μmol) was added to a solution of 11-methyl-3,5-dioxo-1¹H-2,6-diaza-1(2,1)-benzo[d]imidazola-4(1,3)-benzenacycloundecaphane-1⁷-carboxylic acid (62 mg, 153 μmol) and N,N-diisopropylethylamine in DMF (10 ml) at rt. The reaction mixture was stirred for 5 min before (1R,4R)-2-methyl-2,5-diazabicyclo[2.2.1]heptane (42 mg, 230 μmol) was added. The reaction mixture was stirred at rt for 1 h. The solution was concentrated (to ˜ 4 mL), diluted with water (˜2 mL) and purified reverse phase chromatography (C18 column, prep-HPLC, 20-35% MeCN in water containing 0.1% TFA). The over 20 min, then 5 min at highest elution strength. Fractions containing product were collected and freeze-dried to white solids (38.5 mg, 41%). The diasteromers were separated on an IA column using 2% EtOH in CH₂Cl₂ with 0.1% diethylamine. First eluting peak (epimer 1) and second eluting peak (epimer 2) were pooled and concentrated, then redissolved in MeCN and concentrated to solids and dried under reduced pressure overnight.

### Example 29, Diastereomer 1: 12.0 mg as White Solid

¹H NMR (DMSO-d₆) δ: 12.75 (s, 1H), 8.77 (s, 1H), 8.19 (s, 1H), 8.11 (d, 1H), 7.88-7.70 (m, 2H), 7.59 (m, 2H), 7.25 (m, 1H), 7.15 (d, 1H), 4.85-1.20 (m, 23H); LC/MS: M+H=501.3.

### Example 30, Diastereomer 2: 11.2 mg as White Solid

¹H NMR (DMSO-d₆) δ: 12.76 (s, 1H), 8.78 (s, 1H), 8.10 (m, 2H), 7.88-7.69 (m, 2H), 7.59 (m, 2H), 7.24 (m, 1H), 7.18-7.06 (m, 1H), 4.80-1.20 (m, 23H); LC/MS: M+H=501.3.

The absolute stereochemistry of Example 30 was determined using x-ray crystallography to be (S). From this, it follows that Example 29 is (R).

### Examples 31 and 32: (E)-N,N,11-trimethyl-3,5-dioxo-1²,1³-dihydro-1¹H-2,6-diaza-1(2,1)-benzo[d]imidazola-4(1,3)-benzenacycloundecaphane-1⁷-carboxamide

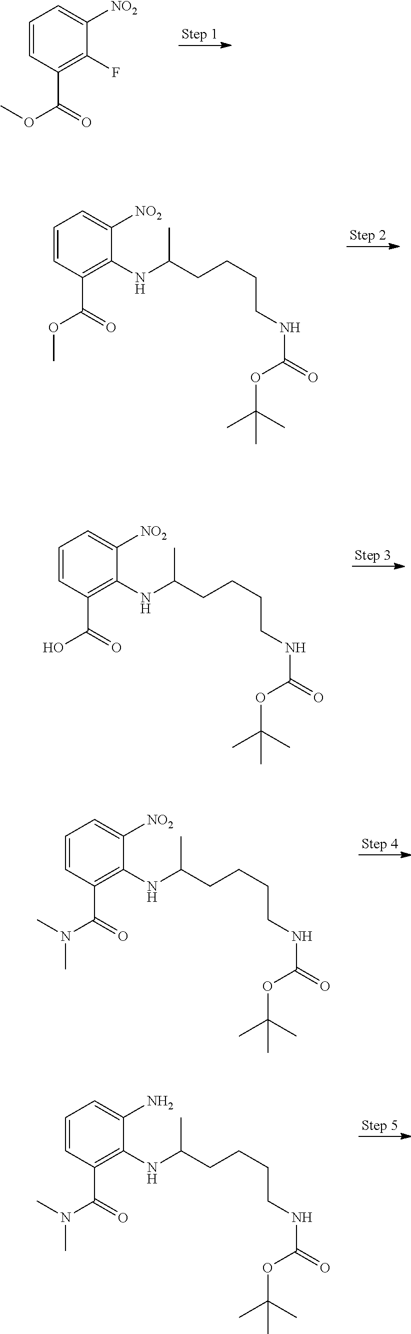 [SMILES: COC(=O)c1cccc([N+](=O)[O-])c1F ; COC(=O)c1cccc([N+](=O)[O-])c1NC(C)CCCCNC(=O)OC(C)(C)C ; CC(CCCCNC(=O)OC(C)(C)C)Nc1c(C(=O)O)cccc1[N+](=O)[O-] ; CC(CCCCNC(=O)OC(C)(C)C)Nc1c(C(=O)N(C)C)cccc1[N+](=O)[O-] ; CC(CCCCNC(=O)OC(C)(C)C)Nc1c(N)cccc1C(=O)N(C)C]

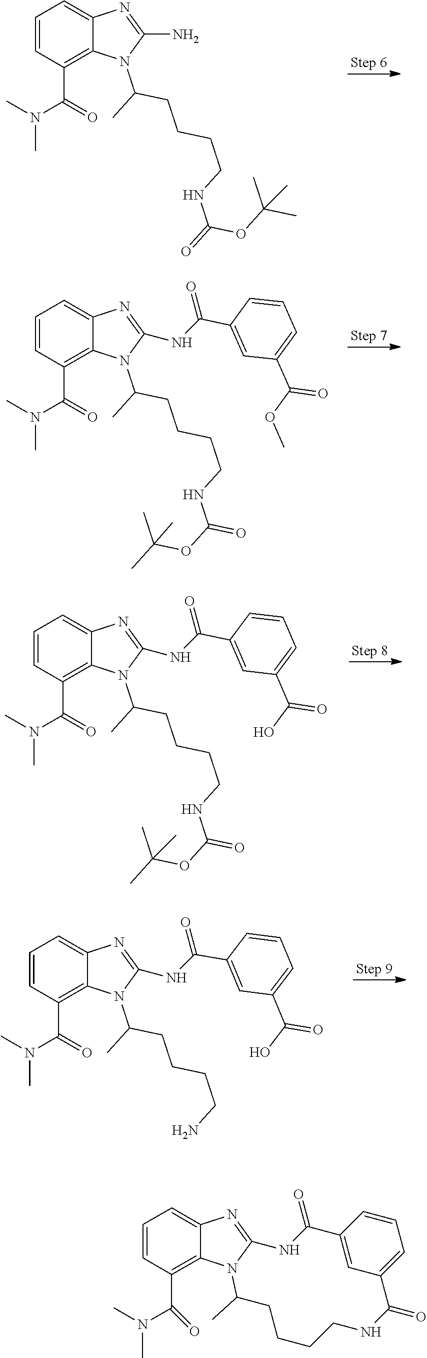

### Step 8: Preparation of methyl 2-((6-((tert-butoxycarbonyl)amino)hexan-2-yl)amino)-3-nitrobenzoate

A solution of methyl 2-fluoro-3-nitrobenzoate (1.00 g, 5.02 mmol), tert-butyl N-(5-aminohexyl)carbamate (1.09 g, 5.02 mmol) and N,N-diisopropylethylamine (2.78 mL, 15.1 mmol) in DMF (10 ml) was heated at 80° C. for 5 h. The reaction mixture was quenched with water and extracted with EtOAc. The organic phase was dried (Na₂SO₄) and concentrated in vacuo. The crude residue was used in the next step without further purification.

¹H NMR (DMSO-d₆) δ: 8.05 (d, 1H) m 8.03 (d, 1H), 6.83 (m, 1H), 6.74 (m, 1H), 3.86 (s, 3H), 3.15 (m, 1H), 2.87 (m, 2H), 1.45 (m, 2H), 1.05 d, 3H); LC/MS: M+H=396.16.

### Step 2: Preparation of 2-((6-((tert-butoxycarbonyl)amino)hexan-2-yl)amino)-3-nitrobenzoic acid

A solution of methyl 2-((6-((tert-butoxycarbonyl)amino)hexan-2-yl)amino)-3-nitrobenzoate (2.0 g, 4.05 mmol) and LiOH hydrate (849 mh, 20.2 mmol) in THF (15 ml) and water (2.0 ml) was stirred at rt for 16 h. After completion the reaction mixture was concentrated under reduced pressure. The crude residue was diluted with water (15 ml), cooled to 0° C. and acidified (pH−4) with 1 N HCl. The precipitate was collected by filtration, washed with cold water and dried in vacuo to give 2-((6-((tert-butoxycarbonyl)amino)hexan-2-yl)amino)-3-nitrobenzoic acid (1.50 g, yield 50%) as a white solid.

LC/MS: M+H=382.17, Rt=1.88 min.

### Step 3: Preparation of tert-butyl (5-((2-(dimethylcarbamoyl)-6-nitrophenyl)amino)hexyl)carbamate

To a stirred solution of 2-((6-((tert-butoxycarbonyl)amino)hexan-2-yl)amino)-3-nitrobenzoic acid (1.50 g, 3.74 mmol) in DMF (10 ml) was added N,N-diisopropylethylamine (3.97 ml, 22.4 mmol) and HATU (2.84 g, 7.47 mmol) 0° C. and stirred for 3 h at RT. The reaction mixture was quenched with ice-cold water and extracted with EtOAc. The organic layer was washed with brine, dried over Na₂SO₄ and concentrated under reduced pressure to get tert-butyl (5-((2-(dimethylcarbamoyl)-6-nitrophenyl)amino)hexyl)carbamate as yellow solid (1.90 g, 88%).

¹H NMR (DMSO-d₆) δ: 8.10 (d, 1H), 7.95 (s, 1H), 7.52 (d, 1H), 7.38 (d, 1H), 6.82 (m, 1H), 6.74 (m, 1H), 3.46 (m, 1H), 2.93 (s, 3H), 2.77 (s, 3H), 1.43 (m, 1H), 1.35 (s, 9H), 1.08 (d, 3H); LC/MS: M+H=409.22.

### Step 4: Preparation of tert-butyl (5-((2-amino-6-(dimethylcarbamoyl)phenyl)amino)hexyl)carbamate

To a stirred solution of tert-butyl (5-((2-(dimethylcarbamoyl)-6-nitrophenyl)amino)hexyl)carbamate (1.90 g, 3.30 mmol) in methanol (30.0 mL) was added 10% Pd/C (211 mg, 1.98 mmol). The reaction mixture was stirred for 4 h at RT under a hydrogen atmosphere. The progress reaction progress was monitored by TLC. After completion of reaction mixture was filtered through celite, washed with EtOAc and concentrated under reduced pressure to get crude. The crude was purified by column chromatography (silica gel, 50% EtOAc/heptane) to afford tert-butyl (5-((2-amino-6-(dimethylcarbamoyl)phenyl)amino)hexyl)carbamate (1.20 g, 87%) as colourless liquid.

¹H NMR (DMSO-d₆) δ: 6.77 (m, 1H), 6.68 (m, 2H), 4.70 (s, 1H), 3.66 (d, 1H), 3.06 (m, 1H), 2.94 (s, 3H), 2.89 (s, 3H), 0.88 (d, 3H); LC/MS: M+H=379.22.

### Step 5: Preparation of tert-butyl (5-(2-amino-7-(dimethylcarbamoyl)-1H-benzo[d]imidazol-1-yl)hexyl)carbamate

To a stirred solution of tert-butyl (5-((2-amino-6-(dimethylcarbamoyl)phenyl)amino)hexyl)carbamate (1.0 g, 2.40 mmol) in methanol (10.0 mL), acetonitrile (5.00 mL) and water (5.00 mL), was added cyanogen bromide (280 mg, 2.64 mmol) and stirred for 4 h at 55° C. The reaction mixture was concentrated under reduced pressure, quenched with ice-cold water and extracted with EtOAc. The organic layer was washed with brine solution, dried over Na₂SO₄ and concentrated under reduced pressure to afford tert-butyl (5-(2-amino-7-(dimethylcarbamoyl)-1H-benzo[d]imidazol-1-yl)hexyl)carbamate (850 mg, 84%) as brown solid.

¹H NMR (DMSO-d₆) δ: 7.13 (d, 1H), 6.95 (m, 1H), 6.71 (m, 1H), 6.64 (d, 1H), 6.18 (s, 1H), 4.23 (m, 1H), 3.04 (s, 3H), 1.87-1.72 (m, 2H), 0.91 (m, 1H); LC/MS: M+H=404.25.

### Step 6: Preparation of methyl 3-((1-(6-((tert-butoxycarbonyl)amino)hexan-2-yl)-7-(dimethylcarbamoyl)-1H-benzo[d]imidazol-2-yl)carbamoyl)benzoate

To a stirred solution of tert-butyl (5-(2-amino-7-(dimethylcarbamoyl)-1H-benzo[d]imidazol-1-yl)hexyl)carbamate (850 mg, 2.02 mmol), 1-methyl-1H-imidazole (830 mg, 10.1 mmol) and (methoxycarbonyl)benzoic acid (546 mg, 3.03 mmol) in MeCN (10.0 ml) was added TCFH (1.42 g, 5.06 mmol) at 0° C. and the reaction mixture was stirred at RT for 2 h. After completion, the reaction mixture was quenched with ice-cold water and extracted with EtOAc. The organic layer was washed with brine solution, dried over Na₂SO₄ and concentrated under reduced pressure to get crude residue. The crude was purified by column chromatography (silica gel, 70% EtOAc/heptane) to afford methyl 3-((1-(6-((tert-butoxycarbonyl)amino)hexan-2-yl)-7-(dimethylcarbamoyl)-1H-benzo[d]imidazol-2-yl)carbamoyl)benzoate (980 mg, 75%) as off white solid.

¹H NMR (DMSO-d₆) δ: 8.80 (s, 1H), 8.42 (d, 1H), 8.09 (d, 1H), 7.63 (m, 2H), 7.26 (m, 1H), 7.08 (m, 1H), 6.70 (m, 1H), 4.31 (m, 1H), 3.90 (s, 3H), 3.10 (s, 3H), 2.69 (s, 3H), 1.65 (m, 2H), 1.02 (m, 1H); LC/MS: M+H=566.23.

### Step 7: Preparation of 3-((1-(6-((tert-butoxycarbonyl)amino)hexan-2-yl)-7-(dimethylcarbamoyl)-1H-benzo[d]imidazol-2-yl)carbamoyl)benzoic acid

To a stirred solution of methyl 3-((1-(6-((tert-butoxycarbonyl)amino)hexan-2-yl)-7-(dimethylcarbamoyl)-1H-benzo[d]imidazol-2-yl)carbamoyl)benzoate (980 mg, 1.52 mmol) in THF (10.0 ml) and water (2.00 mL) was added potassium hydroxide (171 mg, 3.05 mmol) at 0° C. The reaction mixture was stirred 70° C. for 4 h. After completion, the reaction mixture was concentrated under reduced pressure. The reaction mixture pH was adjusted to ˜5 by the addition of 2M HCl. The precipitate was collected by filtration to give 3-((1-(6-((tert-butoxycarbonyl)amino)hexan-2-yl)-7-(dimethylcarbamoyl)-1H-benzo[d]imidazol-2-yl)carbamoyl)benzoic acid (810 mg, 79%) as an off white solid.

¹H NMR (DMSO-d₆) δ: 8.78 (s, 1H), 8.40 (d, 1H), 8.08 (d, 1H), 7.63 (m, 2H), 7.25 (m, 1H), 7.07 (m, 1H), 6.68 (m, 1H), 4.30 (m, 1H), 3.09 (s, 3H), 2.68 (s, 3H), 1.98 (m, 1H), 1.65 (m, 2H), 1.00 (m, 1H); LC/MS: M+H=552.34.

### Step 8: Preparation of 3-((1-(6-aminohexan-2-yl)-7-(dimethylcarbamoyl)-1H-benzo[d]imidazol-2-yl)carbamoyl)benzoic acid

To a stirred solution of 3-((1-(6-((tert-butoxycarbonyl)amino)hexan-2-yl)-7-(dimethylcarbamoyl)-1H-benzo[d]imidazol-2-yl)carbamoyl)benzoic acid (600 mg, 957 μmol) in CH₂Cl₂ (10.0 ml) was added 4 M HCl (5.0 ml). The reaction mixture was stirred at rt for 3 h and then concentrated under vacuum to get 3-((1-(6-aminohexan-2-yl)-7-(dimethylcarbamoyl)-1H-benzo[d]imidazol-2-yl)carbamoyl)benzoic acid (450 mg, 99%), which was used in next step without any further purification.

¹H NMR (DMSO-d₆) δ: 879 (s, 1H), 8.40 (d, 1H), 8.09 (d, 1H), 7.79 (m, 2H), 7.65 (m, 2H), 7.27 (m, 1H), 7.06 (m, 1H), 3.10 (s, 3H), 2.00 (m, 1H), 1.50 (m, 2H); LC/MS: M−H=450.22.

### Step 9: Preparation of N,N,11-trimethyl-3,5-dioxo-1¹H-2,6-diaza-1(2,1)-benzo[d]imidazola-4(1,3)-benzenacycloundecaphane-1⁷-carboxamide

To a stirred solution of 3-((1-(6-aminohexan-2-yl)-7-(dimethylcarbamoyl)-1H-benzo[d]imidazol-2-yl)carbamoyl)benzoic acid (400 mg, 850 μmol) in MeCN (500 ml) and DMF (30.0 ml) was added TCFH (597 mg, 2.13 mmol). The resulting solution was added a solution of 1-methyl-1H-imidazole (339 μL, 4.25 mmol) in MeCN (0.5 ml) at 0° C. The reaction mixture was stirred at rt for 1 h. After completion, the reaction mixture was concentrated under reduced pressure. The crude was purified using preparative HPLC to give racemic N,N,11-trimethyl-3,5-dioxo-1¹H-2,6-diaza-1(2,1)-benzo[d]imidazola-4(1,3)-benzenacycloundecaphane-1⁷-carboxamide (43 mg, 12%). The enantiomers were separated using chiral chromatography.

### Example 31, Enantiomer 1

¹H NMR (DMSO-d₆) δ: 8.75 (m, 1H), 8.10 (d, 1H), 7.68 (m, 2H), 7.53 (m, 2H), 7.13 (bs, 1H), 6.91 (bs, 1H), 3.06 (s, 3H), 2.84 (s, 3H); LC/MS: M+H=434.19.

Chiralpak-IG (4.6×250 mm) 5μ, 0.1% TEA in n-Hexane:EtOH=(20:80) (v/v), flow rate: 1 ml/min, Rt=21.96 min

### Example 32, Enantiomer 2

¹H NMR (DMSO-d₆) (T=353 K) δ: 8.84 (bs, 1H), 8.07 (d, 1H,), 7.62 (d, 1H), 7.48 (m, 2H), 7.36 (m, 1H), 7.02 (m, 1H), 6.77 (d, 1H), 3.50 (m, 1H), 2.84 (s, 3H), 1.75 (m, 2H), 1.55 (m, 3H); LC/MS: M+H=434.45.

Chiralpak-IG (4.6×250) 5μ 0.1% TEA in n-Hexane/EtOH=20:80 (v/v), flow rate: 1 ml/min, Rt=29.40 min.

The absolute stereochemistry of Example 32 was confirmed to be (S) through an independent synthesis starting from (S,E)-11-methyl-3,5-dioxo-1²,1³-dihydro-1¹H-2,6-diaza-1(2,1)-benzo[d]imidazola-4(1,3)-benzenacycloundecaphane-1⁷-carboxylic acid and dimethyl amine.

### Example 33: (E)-3-(11-methyl-3,5-dioxo-1²,1³-dihydro-1¹H-2,6-diaza-1(2,1)-benzo[d]imidazola-4(1,3)-benzenacycloundecaphane-1⁷-yl)pyridine 1-oxide

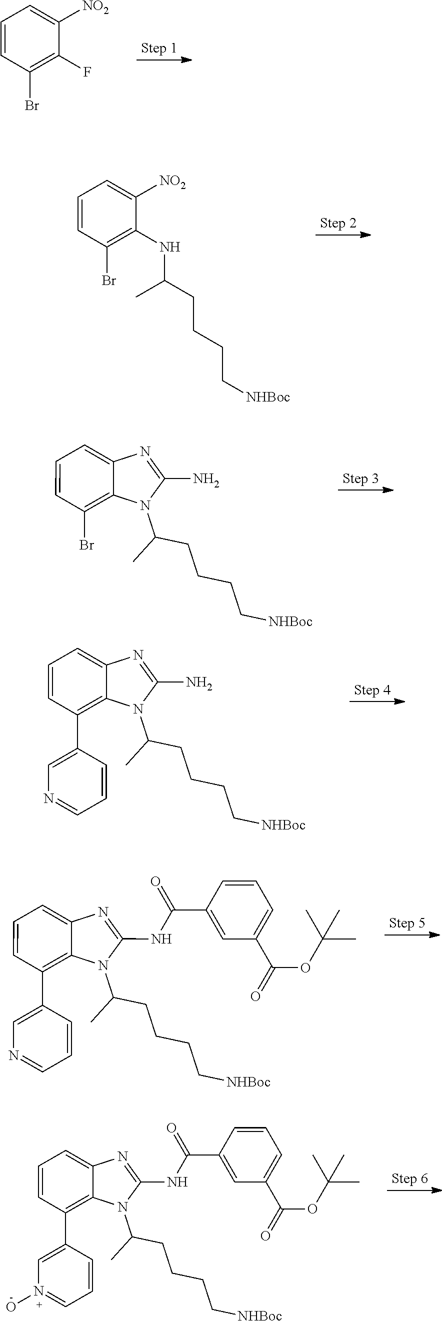

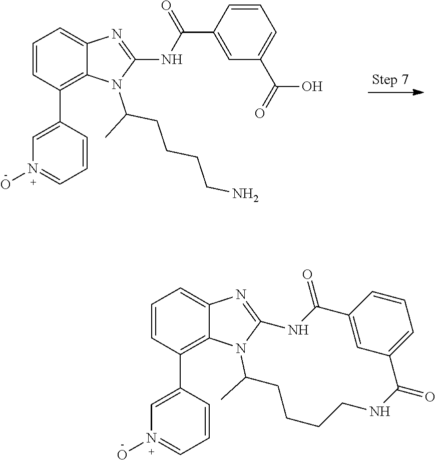

### Step 1: Preparation of tert-butyl (5-((2-bromo-6-nitrophenyl)amino)hexyl)carbamate

A mixture of 1-bromo-2-fluoro-3-nitrobenzene (1.0 g, 4.55 mmol), tert-butyl (5-aminohexyl)carbamate (1.03 g, 4.77 mmol) and K₂CO₃ (1.26 g, 9.09 mmol) in MeCN (30 ml) was stirred at rt for 2 days, filtered through celite and the filtrate was evaporated to dryness. tert-butyl (5-((2-bromo-6-nitrophenyl)amino)hexyl)carbamate was used without further purification.

### Step 2: Preparation of tert-butyl (5-(2-amino-7-bromo-1H-benzo[d]imidazol-1-yl)hexyl)carbamate

To SnCl₂ (3.19 g, 16.8 mmol) was added to a solution of of tert-butyl (5-((2-bromo-6-nitrophenyl)amino)hexyl)carbamate (1.40 g, 3.36 mmol) in EtOH (35 ml) under Ar. The reaction mixture was heated to 80° C. in a closed vial and stirred for 40 min. The mixture was cooled and evaporated to dryness. The residue was dissolved in EtOAc (50 ml) and 2.5 M NaOH (50 ml) was added and the mixture was stirred for 5 minutes under argon. Celite was added and the mixture filtered through a plug of celite. The filtrate was evaporated to dryness. The residue was dissolved in MeOH (25 ml) and water (5 ml) and cyanogen bromide (427 mg, 4.04 mmol) was added. The reaction mixture was stirred under N₂ at RT overnight. The volatiles were removed under reduced pressure and the residue purified by flash chromatography on silica gel (Biotage 12 g using 50-100% EtOAc in pet. Ether). The crude product (712 mg) was used without further purification.

### Step 3: Preparation of tert-butyl (5-(2-amino-7-(pyridin-3-yl)-1H-benzo[d]imidazol-1-yl)hexyl)carbamate

To Pd(PPh₃)₄(13 mg, 11 μmol) was added to a degassed solution of tert-butyl (5-(2-amino-7-bromo-1H-benzo[d]imidazol-1-yl)hexyl)carbamate (45 mg, 109 μmol) and 3-(tributylstannyl)pyridine (54 mg, 148 μmol) in a vial. The vial was sealed and heated at 120° C. for 36 h. The reaction mixture was purified using preparative HPLC (10-30% MeCN/H₂O with 0.1% TFA). The fractions were concentrated and dissolved in CH₂Cl₂, washed with NaHCO₃ sat. to give tert-butyl (5-(2-amino-7-(pyridin-3-yl)-1H-benzo[d]imidazol-1-yl)hexyl)carbamate (17 mg, 38%).

¹H NMR (DMSO-d₆) δ: 8.73-8.69 (m, 2H), 8.69 (m, 1H), 7.77 (m, 1H), 7.48 (d, 1H), 7.42 (m, 1H), 7.18 (m, 1H), 6.85 (d, 1H), 4.70 (s, 2H), 3.97 (m, 1H), 2.98 (m, 6.3 Hz, 2H), 1.94-1.80 (m, 1H), 1.60 (d, 1H), 1.45 (s, 9H), 1.36-1.22 (m, 5H), 1.01 (s, 1H), 0.90-0.75 (m, 1H).

### Step 4: Preparation of tert-butyl 3-((1-(6-((tert-butoxycarbonyl)amino)hexan-2-yl)-7-(pyridin-3-yl)-1H-benzo[d]imidazol-2-yl)carbamoyl)benzoate

N,N-Diisopropylethylamine (59 μl, 342 μmol) was added to a solution of HATU (71 mg, 188 μmol) and 3-(tert-butoxycarbonyl)benzoic acid (42 mg, 188 μmol) in MeCN (0.5 ml). The solution was stirred for 5 min and a solution tert-butyl (5-(2-amino-7-(pyridin-3-yl)-1H-benzo[d]imidazol-1-yl)hexyl)carbamate (70 mg, 171 μmol) in MeCN (2.0 ml) was added. The resulting solution was stirred for 2 h. The reaction was purified by reverse phase chromatography (C18 column, prep-HPLC, using 5-20% MeCN in water containing 0.1% NH₃) to give tert-butyl 3-((1-(6-((tert-butoxycarbonyl)amino)hexan-2-yl)-7-(pyridin-3-yl)-1H-benzo[d]imidazol-2-yl)carbamoyl)benzoate as a white solid (70 mg, 67%).

¹H NMR (DMSO-d₆) δ: 12.86 (s, 1H), 8.90 (s, 1H), 8.73 (d, 2H), 8.42 (d, 1H), 8.13 (d, 1H), 7.77 (d, 1H), 7.51 (m, 2H), 7.38 (d, 1H), 7.30 (d, 1H), 7.06 (s, 1H), 3.91 (s, 1H), 2.96 (s, 2H), 2.50 (s, 1H), 1.62 (s, 9H), 1.33 (d, 9H); LC/MS: M+H=614.3.

### Step 5: Preparation of 3-(1-(6-((tert-butoxycarbonyl)amino)hexan-2-yl)-2-(3-(tert-butoxycarbonyl)benzamido)-1H-benzo[d]imidazol-7-yl)pyridine 1-oxide

mCPBA was added to a solution of tert-butyl 3-((1-(6-((tert-butoxycarbonyl)amino)hexan-2-yl)-7-(pyridin-3-yl)-1H-benzo[d]imidazol-2-yl)carbamoyl)benzoate (70 mg, 114 μmol) in CH₂Cl₂ (4.0 ml) under air at RT. The reaction mixture was stirred overnight. The reaction mixture was extracted with NaHCO₃ (aq, sat), dried and concentrated. 3-(1-(6-((tert-butoxycarbonyl)amino)hexan-2-yl)-2-(3-(tert-butoxycarbonyl)benzamido)-1H-benzo[d]imidazol-7-yl)pyridine 1-oxide (77 mg) was used without further purification.

¹H NMR (DMSO-d₆) δ: 12.91 (s, 1H), 8.91 (s, 1H), 8.43 (d, 1H), 8.36 (d, 2H), 8.16 (d, 1H), 7.53 (m, 1H), 7.47-7.40 (m, 2H), 7.36 (d, 1H), 7.31 (, 1H), 7.08-7.01 (m, 1H), 3.99 (s, 1H), 3.00 (s, 2H), 2.62-2.29 (m, 1H), 2.07 (s, 1H), 1.64 (s, 9H), 1.42 (d, 1¹H), 1.14 (s, 1H), 0.98 (s, 1H).

### Step 6: Preparation of 3-(1-(6-aminohexan-2-yl)-2-(3-carboxybenzamido)-1H-benzo[d]imidazol-7-yl)pyridine 1-oxide

3-(1-(6-((tert-butoxycarbonyl)amino)hexan-2-yl)-2-(3-(tert-butoxycarbonyl)benzamido)-1H-benzo[d]imidazol-7-yl)pyridine 1-oxide (72 mg, 114 μmol) was dissolved in dioxane (2.0 ml) and 5 M aqueous HCl (2.0 ml) was added. The reaction mixture was stirred for 70 h at rt. The reaction was concentrated under reduced pressure and the residue was dissolved in MeCN and evaporated to dryness. The product was used without further purification.

### Step 7: Preparation of 3-(11-methyl-3,5-dioxo-1¹H-2,6-diaza-1(2,1)-benzo[d]imidazola-4(1,3)-benzenacycloundecaphane-1⁷-yl)pyridine 1-oxide

HATU (65 mg, 171 μmol) and N,N-diisopropylethylamine (98 μl, 569 μmol) were added to a solution of 3-(1-(6-aminohexan-2-yl)-2-(3-carboxybenzamido)-1H-benzo[d]imidazol-7-yl)pyridine 1-oxide in THF (250 ml). The reaction mixture was stirred overnight at RT and HATU (40 mg, 171 μmol) and N,N-diisopropylethylamine (98 μl, 569 μmol) were added. The reaction mixture was refluxed overnight, cooled to RT and filtered. The filtrate was concentrated to dryness under reduced pressure. The residue was purified by reverse phase chromatography (C18 column, prep-HPLC, using 5-20% MeCN in water containing 0.1% NH₃) to give 3-(11-methyl-3,5-dioxo-1¹H-2,6-diaza-1(2,1)-benzo[d]imidazola-4(1,3)-benzenacycloundecaphane-1⁷-yl)pyridine 1-oxide as a white powder (3.5 mg, 7%).

¹H NMR (DMSO-d₆) δ: 8.72 (s, 1H), 8.46 (d, 1H), 8.36 (d, 1H), 8.12 (d, 1H), 7.75 (d, 2H), 7.61 (m, 2H), 7.51 (d, 2H), 7.28 (m, 1H), 7.09 (d, 1H), 4.10 (s, 1H), 3.57 (s, 1H), 2.96 (s, 2H), 1.88 (d, 1H), 1.71 (s, 1H), 1.52 (s, 4H), 1.43-1.15 (m, 3H); LC/MS.

M+H=456.2.

### Example 34: (E)-N,11-dimethyl-3,5-dioxo-1²,1³-dihydro-1¹H-2,6-diaza-1(2,1)-benzo[d]imidazola-4(1,3)-benzenacycloundecaphane-1⁷-carboxamide

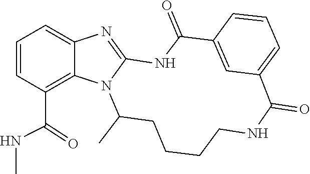 [SMILES: CNC(=O)c1cccc2nc3n(c12)C(C)CCCCNC(=O)c1cccc(c1)C(=O)N3]

### Preparation of N,11-dimethyl-3,5-dioxo-1¹H-2,6-diaza-1(2,1)-benzo[d]imidazola-4(1,3)-benzenacycloundecaphane-1⁷-carboxamide

HATU (12 mg, 32 μmol) was added to a solution of 11-methyl-3,5-dioxo-1¹H-2,6-diaza-1(2,1)-benzo[d]imidazola-4(1,3)-benzenacycloundecaphane-1⁷-carboxylic acid (12 mg, 29 μmol), N,N-diisopropylethylamine (15 μl, 88 μmol) and 2 M methylamine in THF (44 μl, 88 μmol) in DMF (1.0 ml) at rt. The reaction mixture was stirred at rt for 40 min, diluted with water (1 ml) and purified by reverse phase chromatography (18 column, 5-40% MeCN in water containing 0.1% TFA) to give N,11-dimethyl-3,5-dioxo-1¹H-2,6-diaza-1(2,1)-benzo[d]imidazola-4(1,3)-benzenacycloundecaphane-1⁷-carboxamide as a white solid (4.8 mg, 39%).

¹H NMR (DMSO-d₆) δ: 12.77 (s, 1H), 8.81 (s, 1H), 8.75-8.68 (m, 1H), 8.11 (d, 1H), 7.83 (s, 1H), 7.75 (d, 1H), 7.64-7.54 (m, 2H), 7.22 (m, 1H), 7.16 (d, 1H), 4.83 (s, 1H), 3.05 (s, 2H), 2.81 (d, 3H), 1.98-1.35 (m, 9H); LC/MS: M+H=420.0.

### Examples 35 and 36: (E)-11-methyl-1⁷-(2-oxo-1,2-dihydropyridin-3-yl)-1²,1³-dihydro-1¹H-2,6-diaza-1(2,1)-benzo[d]imidazola-4(1,3)-benzenacycloundecaphane-3,5-dione

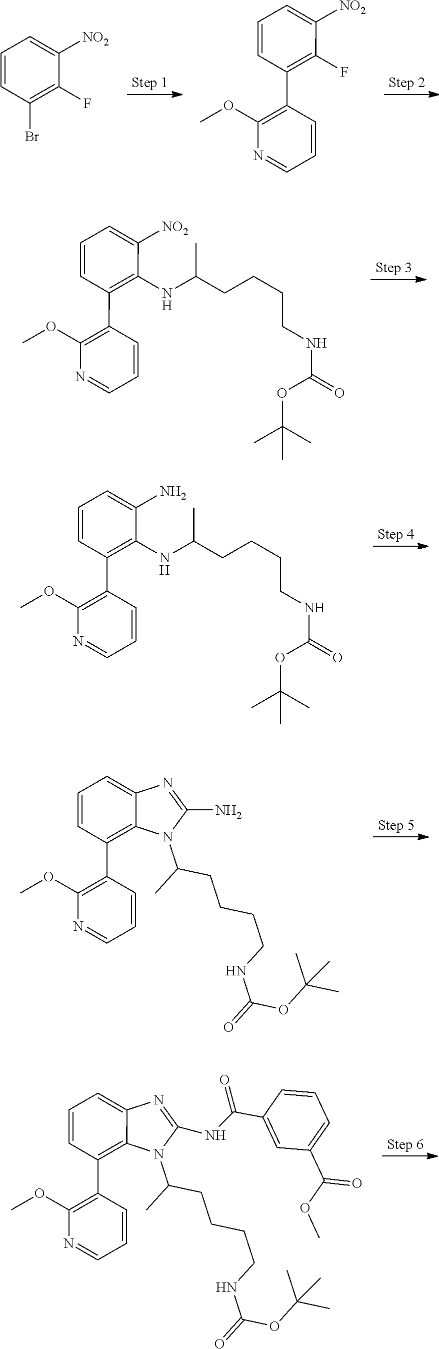

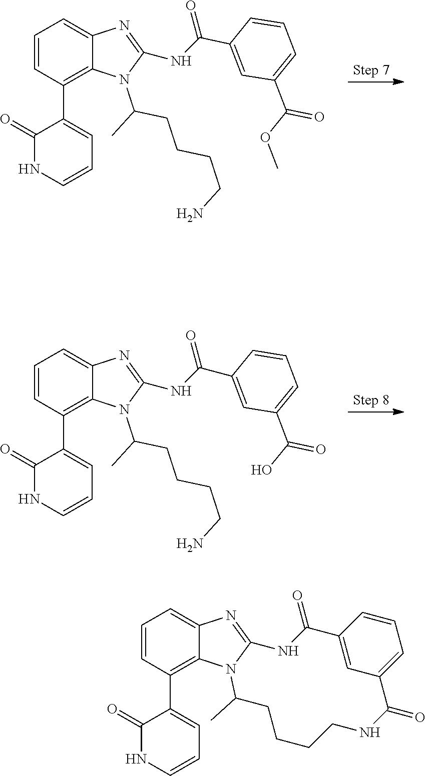 [SMILES: COC(=O)c1cccc(C(=O)Nc2nc3cccc(-c4ccc[nH]c4=O)c3n2C(C)CCCCN)c1 ; CC(CCCCN)n1c(NC(=O)c2cccc(C(=O)O)c2)nc2cccc(-c3ccc[nH]c3=O)c21 ; CC1CCCCNC(=O)c2cccc(c2)C(=O)Nc2nc3cccc(-c4ccc[nH]c4=O)c3n21]

### Step 1: Preparation of 3-(2-fluoro-3-nitrophenyl)-2-methoxypyridine

A solution of 1-bromo-2-fluoro-3-nitrobenzene (2.00 g, 9.09 mmol), (2-methoxypyridin-3-yl)boronic acid (4.17 g, 27.3 mmol) and tripotassium phosphate (5.79 g, 27.3 mmol) in 1,4-dioxane (10.0 mL) and water (1.00 mL) was purged with N₂ for 10 min. To the reaction mixture was added tetrakis(triphenylphosphane) palladium (1.05 g, 909 μmol) and the reaction mixture was heated at 130° C. for 2 h. The reaction mixture was quenched with water. The aqueous layer was extracted with EtOAc, dried (Na₂SO₄) and concentrated in vacuo. The crude residue was purified by flash chromatography (silica gel, 0 to 15% EtOAc in hexane) to give 3-(2-fluoro-3-nitrophenyl)-2-methoxypyridine (2.40 g, 79%) as a white solid.

¹H NMR (DMSO-d₆) δ: 8.31 (m, 1H), 8.19 (m, 1H), 7.83 (m, 1H), 7.80 (m, 1H), 7.52 (m, 1H), 7.16 (m, 1H), 3.87 (s, 3H); LC/MS: M+H=249.05.

### Step 2: Preparation of tert-butyl (5-((2-(2-methoxypyridin-3-yl)-6-nitrophenyl)amino)hexyl)carbamate

A solution of 3-(2-fluoro-3-nitrophenyl)-2-methoxypyridine (2.00 g, 8.06 mmol), tert-butyl N-(5-aminohexyl)carbamate (1.74 g, 8.06 mmol) and N,N-diisopropylethylamine (4.46 mL, 24.2 mmol) in DMF (2.0 ml) was heated at 90° C. for 16 h. The reaction mixture was quenched with water. The aqueous layer was extracted with EtOAc, dried (Na₂SO₄) and concentrated in vacuo. The crude product was used without further purification.

LC/MS: M+H=445.08.

### Step 3: Preparation of tert-butyl (5-((2-amino-6-(2-methoxypyridin-3-yl)phenyl)amino)hexyl)carbamate

A solution of tert-butyl (5-((2-(2-methoxypyridin-3-yl)-6-nitrophenyl)amino)hexyl)carbamate (3.00 g, 4.32 mmol) and 10% Pd/C (palladium (2.00 g, 1.88 mmol) in MeOH (60 ml) was stirred under H₂ (1 atm) at rt for 3 h. The reaction mixture was concentrated in vacuo to give tert-butyl (5-((2-amino-6-(2-methoxypyridin-3-yl)phenyl)amino)hexyl)carbamate (2.30 g) as an off white solid that was used without further purification.

¹H NMR (DMSO-d₆) δ: 8.17 (m, 1H), 7.95 (s, 1H), 7.55 (m, 1H), 6.75 (m, 1H), 6.65 (m, 1H), 6.33 (m, 1H), 3.83 (s, 3H), 3.17 (m, 1H), 2.73 (m, 1H), 1.37 (m, 9H);

LC/MS: M+H=415.33.

### Step 4: Preparation of tert-butyl (5-(2-amino-7-(2-methoxypyridin-3-yl)-1H-benzo[d]imidazol-1-yl)hexyl)carbamate

To a stirred solution of tert-butyl (5-((2-amino-6-(2-methoxypyridin-3-yl)phenyl)amino)hexyl)carbamate (2.30 g, 4.27 mmol) in methanol (10.0 ml), MeCN (5.0 ml) and water (5.0 ml) was added cyanogen bromide. The reaction mixture was stirred for 8 h at 50° C. After completion, the reaction mixture was quenched with ice-cold water and extracted with EtOAc. The organic layer was washed with brine solution, dried over Na₂SO₄ and concentrated under reduced pressure to get crude residue. The crude was used without further purification.

¹H NMR (DMSO-d₆) δ: 8.23 (m, 1H), 7.66 (m, 1H), 7.12 (m, 2H), 6.94 (m, 1H), 6.63 (d, 1H), 3.91 (s, 3H), 2.69 (m, 1H), 1.34 (s, 9H); LC/MS: M+H=440.18.

### Step 5: Preparation of methyl 3-((1-(6-((tert-butoxycarbonyl)amino)hexan-2-yl)-7-(2-methoxypyridin-3-yl)-1H-benzo[d]imidazol-2-yl)carbamoyl)benzoate

To a stirred solution of tert-butyl (5-(2-amino-7-(2-methoxypyridin-3-yl)-1H-benzo[d]imidazol-1-yl)hexyl)carbamate, (methoxycarbonyl)benzoic acid (822 mg, 4.56 mmol) and 1-methyl-1H-imidazole (1.52 mL, 19.0 mmol) in MeCN (60 ml) was added TCFH (2.13 g, 7.61 mmol) at 0° C. The reaction mixture was stirred at RT for 4 h. The reaction mixture was quenched with water. The aqueous layer was extracted with EtOAc, dried (Na₂SO₄) and concentrated in vacuo. The crude residue was purified by flash chromatography (silica gel, 5% to 80% EtOAc in hexane) to give methyl 3-((1-(6-((tert-butoxycarbonyl)amino)hexan-2-yl)-7-(2-methoxypyridin-3-yl)-1H-benzo[d]imidazol-2-yl)carbamoyl)benzoate (2.20 g, 88%) as a solid.

¹H NMR (DMSO-d₆) δ: 8.78 (s, 1H), 8.41 (d, 1H), 8.33 (m, 1H), 8.08 (d, 1H), 7.79 (m, 1H), 7.64 (m, 2H), 7.26 (m, 1H), 7.18 (m, 1H), 6.97 (d, 1H), 3.88 (s, 3H), 3.82 (s, 3H), 1.58 (d, 2H), 1.31 (s, 9H); LC/MS: M+H=602.11.

### Step 6: Preparation of methyl 3-((1-(6-aminohexan-2-yl)-7-(2-oxo-1,2-dihydropyridin-3-yl)-1H-benzo[d]imidazol-2-yl)carbamoyl)benzoate

To a stirred solution of methyl 3-((1-(6-((tert-butoxycarbonyl)amino)hexan-2-yl)-7-(2-methoxypyridin-3-yl)-1H-benzo[d]imidazol-2-yl)carbamoyl)benzoate (1.30 g, 1.86 mmol) and sodium iodide (557 mg, 3.72 mmol) in MeCN (50 ml) was added chlorotrimethylsilane (475 μL, 3.72 mmol) dropwise at rt and the reaction mixture was heated at 55° C. for 16 h. After completion, the reaction mixture was quenched with sat aq NaHCO₃. The aqueous layer was extracted with 10% MeOH in CH₂Cl₂, dried (Na₂SO₄) and concentrated in vacuo to give methyl 3-((1-(6-aminohexan-2-yl)-7-(2-oxo-1,2-dihydropyridin-3-yl)-1H-benzo[d]imidazol-2-yl)carbamoyl)benzoate (1.10 g, 92%) as a solid.

¹H NMR (DMSO-d₆) δ: 8.80 (s, 1H), 8.41 (d, 1H), 8.08 (d, 1H), 7.58 (m, 4H), 7.25 (m, 1H), 6.96 (d, 1H), 6.35 (m, 1H), 4.35 (m, 1H), 3.89 (s, 3H), 1.44 (m, 1H), 1.15 (m, 1H); LC/MS: M+H=488.37.

### Step 7: Preparation of 3-((1-(6-aminohexan-2-yl)-7-(2-oxo-1,2-dihydropyridin-3-yl)-1H-benzo[d]imidazol-2-yl)carbamoyl)benzoic acid

A solution of methyl 3-((1-(6-aminohexan-2-yl)-7-(2-oxo-1,2-dihydropyridin-3-yl)-1H-benzo[d]imidazol-2-yl)carbamoyl)benzoate (1.10 g, 1.80 mmol) and sodium hydroxide (217 mg, 3 eq., 5.41 mmol) in methanol (15.0 mL) and water (2.00 mL) was heated at 65° C. for 8 h. The reaction mixture was concentrated in vacuo. The crude residue was triturated with 1 N aq HCl. The precipitate was filtered, washed with cold water and dried in vacuo to give 3-((1-(6-aminohexan-2-yl)-7-(2-oxo-1,2-dihydropyridin-3-yl)-1H-benzo[d]imidazol-2-yl)carbamoyl)benzoic acid (1.0 g, 83%) as an off white solid.

¹H NMR (DMSO-d₆) δ: 8.77 (m, 1H), 8.32 (m, 1H), 8.06 (m, 1H), 7.56 (m, 4H), 7.22 (m, 1H), 6.92 (d, 1H), 6.36 (m, 1H), 4.31 (m, 1H), 2.56 (m, 1H), 1.68) m, 2H), 1.53 (m, 1H); LC/MS: M+H=474.29.

### Step 8: Preparation of 11-methyl-1⁷-(2-oxo-1,2-dihydropyridin-3-yl)-1¹H-2,6-diaza-1(2,1)-benzo[d]imidazola-4(1,3)-benzenacycloundecaphane-3,5-dione

To a stirred solution of 3-((1-(6-aminohexan-2-yl)-7-(2-oxo-1,2-dihydropyridin-3-yl)-1H-benzo[d]imidazol-2-yl)carbamoyl)benzoic acid (200 mg, 359 μmol) and 1-methyl-1H-imidazole (146 μL, 1.80 mmol) in DMF (100 ml) was added TCFH (201 mg, 718 mol) at RT and the reaction mixture was stirred at RT for 1 h. The reaction mixture was quenched with water and the aqueous layer was extracted with 5% MeOH in CH₂Cl₂. This residue was purified by prep-HPLC to give racemic 11-methyl-1⁷-(2-oxo-1,2-dihydropyridin-3-yl)-1¹H-2,6-diaza-1(2,1)-benzo[d]imidazola-4(1,3)-benzenacycloundecaphane-3,5-dione (35 mg, 21%) as a white solid. The enantiomers were separated by chiral chromatography.

### Example 35, Enantiomer 1

¹H NMR (DMSO-d₆) δ: 8.72 (s, 1H), 8.10 (m, 1H), 7.73 (m, 2H), 7.58 (m, 4H), 7.20 (m, 1H), 6.93 (m, 1H), 6.34 (m, 1H), 1.75 (m, 3H); LC/MS: M+H=456.36. Chiralpak-IF (4.6×250) 5 um, Ethyl acetate/IPA=40:60 (v/v), flow rate: 1 ml/min, Rt=7.30.

### Example 36, Enantiomer 2

¹H NMR (DMSO-d₆) δ: 8.72 (s, 1H), 8.11 (m, 1H), 7.73 (m, 2H), 7.58 (m, 4H), 7.20 (m, 1H), 6.94 (m, 1H), 6.34 (m, 1H), 1.75 (m, 3H); LC/MS: M+H=456.38.

Chiralpak-IF (4.6×250) 5 um, Ethyl acetate/IPA=40:60 (v/v), flow rate: 1 ml/min, Rt=6.22.

### Example 37: (E)-1⁷-(3,3-difluoropyrrolidine-1-carbonyl)-11-methyl-1²,1³-dihydro-1¹H-2,6-diaza-1(2,1)-benzo[d]imidazola-4(1,3)-benzenacycloundecaphane-3,5-dione

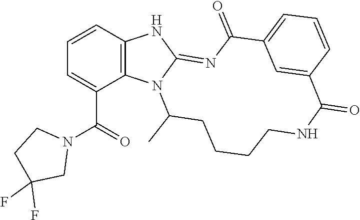 [SMILES: CC1CCCCNC(=O)c2cccc(c2)C(=O)/N=C2\Nc3cccc(C(=O)N4CCC(F)(F)C4)c3N21]

HATU (11 mg, 30 μmol) was added to a solution of 11-methyl-3,5-dioxo-1¹H-2,6-diaza-1(2,1)-benzo[d]imidazola-4(1,3)-benzenacycloundecaphane-1⁷-carboxylic acid (11 mg, 27 μmol), N,N-diisopropylethylamine (28 μl, 165 μmol) and 3,3-difluoropyrrolidine (12 mg, 83 μmol) in DMF (1.0 ml) at rt. The reaction mixture was stirred overnight at RT, diluted with water (1.0 ml) and purified by reverse phase chromatography (C18 column, prep-HPLC, 20-60% MeCN in water containing 0.1% TFA) to give (E)-1⁷-(3,3-difluoropyrrolidine-1-carbonyl)-11-methyl-1²,1³-dihydro-1¹H-2,6-diaza-1(2,1)-benzo[d]imidazola-4(1,3)-benzenacycloundecaphane-3,5-dione as a white solid (9.7 mg, 71%).

¹H NMR (DMSO-d₆) δ: 12.79 (s, 1H), 8.80 (s, 1H), 8.11 (d, 1H), 7.83 (s, 1H), 7.75 (d, 1H), 7.59 (m, 2H), 7.34-7.22 (m, 1H), 7.17 (d, 1H), 4.69-1.14 (m, 18H); LC/MS: M+H=496.0.

### Example 38: methyl (E)-N-methyl-N-(11-methyl-3,5-dioxo-1²,1³-dihydro-1¹H-2,6-diaza-1(2,1)-benzo[d]imidazola-4(1,3)-benzenacycloundecaphane-1⁷-carbonyl)glycinate

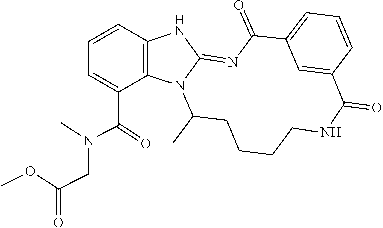 [SMILES: COC(=O)CN(C)C(=O)c1cccc2c1N1/C(=N/C(=O)c3cccc(c3)C(=O)NCCCCC1C)N2]

HATU (11 mg, 29 μmol) was added to a solution of 11-methyl-3,5-dioxo-1¹H-2,6-diaza-1(2,1)-benzo[d]imidazola-4(1,3)-benzenacycloundecaphane-1⁷-carboxylic acid (11 mg, 26 μmol), N,N-diisopropylethylamine (28 μl, 160 μmol) and methyl methylglycinate (11 mg, 80 μmol) in DMF (1.0 ml) at rt. The reaction mixture was stirred overnight at rt, diluted with water (1.0 ml) and purified by reverse phase chromatography (C18 column, prep-HPLC, 20-60% MeCN in water containing 0.1% TFA) to give methyl (E)-N-methyl-N-(11-methyl-3,5-dioxo-1²,1³-dihydro-1¹H-2,6-diaza-1(2,1)-benzo[d]imidazola-4(1,3)-benzenacycloundecaphane-1⁷-carbonyl)glycinate as a white solid (7.9 mg, 61%).

¹H NMR (DMSO-d₆) δ: 12.75 (s, 1H), 8.78 (s, 1H), 8.11 (d, 1H), 7.75 (d, 2H), 7.66-7.52 (m, 2H), 7.33-7.16 (m, 1H), 7.12-6.92 (m, 1H), 4.82-2.65 (m, 1¹H), 2.07-1.13 (m, 9H); LC/MS. M+H=492.0.

### Example 39: (E)-N-cyclobutyl-N,11-dimethyl-3,5-dioxo-1²,1³-dihydro-1¹H-2,6-diaza-1(2,1)-benzo[d]imidazola-4(1,3)-benzenacycloundecaphane-1⁷-carboxamide

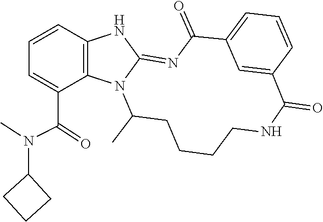 [SMILES: CC1CCCCNC(=O)c2cccc(c2)C(=O)/N=C2\Nc3cccc(C(=O)N(C)C4CCC4)c3N21]

N,N-diisopropylethylamine (28 μl, 160 μmol) was added to a solution of 11-methyl-3,5-dioxo-1¹H-2,6-diaza-1(2,1)-benzo[d]imidazola-4(1,3)-benzenacycloundecaphane-1⁷-carboxylic acid (10 mg, 25 μmol) and HATU (10 mg, 27 μmol) in DMF (0.25 ml) at rt. The solution was stirred for 2 min before N-methylcyclobutanamine (6 mg, 49 μmol). The reaction mixture was stirred overnight at RT, diluted with water (1.0 ml) and purified by reverse phase chromatography (C18 column, prep-HPLC, 20-60% MeCN in water containing 0.1% TFA) to give (E)-N-cyclobutyl-N,11-dimethyl-3,5-dioxo-1²,1³-dihydro-1¹H-2,6-diaza-1(2,1)-benzo[d]imidazola-4(1,3)-benzenacycloundecaphane-1⁷-carboxamide (7.4 mg, 64%).

¹H NMR (DMSO-d₆) δ: 12.76 (s, 1H), 8.78 (s, 1H), 8.12 (d, 1H), 7.76 (d, 2H), 7.59 (m, 2H), 7.25 (m, 1H), 7.00 (m, 1H), 5.74-3.94 (m, 2H), 3.55 (s, 2H), 3.07 (d, 3H), 2.81 (s, 1H), 2.35-2.06 (m, 3H), 1.96-1.28 (m, 1¹H); LC/MS: M+H=474.3.

### Example 40: (E)-1⁷-(3,3-difluoroazetidine-1-carbonyl)-11-methyl-1²,1³-dihydro-1¹H-2,6-diaza-1(2,1)-benzo[d]imidazola-4(1,3)-benzenacycloundecaphane-3,5-dione

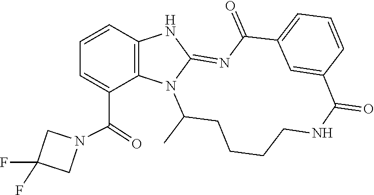 [SMILES: CC1CCCCNC(=O)c2cccc(c2)C(=O)/N=C2\Nc3cccc(C(=O)N4CC(F)(F)C4)c3N21]

N,N-diisopropylethylamine (21 μl, 123 μmol) was added to a solution of 11-methyl-3,5-dioxo-1¹H-2,6-diaza-1(2,1)-benzo[d]imidazola-4(1,3)-benzenacycloundecaphane-1⁷-carboxylic acid (10 mg, 25 μmol) and HATU (10 mg, 27 μmol) in DMF (0.25 ml) at rt. The solution was stirred for 2 min before 3,3-difluoroazetidine (6 mg, 49 μmol). The reaction mixture was stirred overnight at rt, diluted with water (1.0 ml) and purified by reverse phase chromatography (C18 column, prep-HPLC, 20-60% MeCN in water containing 0.1% TFA) to give (E)-1⁷-(3,3-difluoroazetidine-1-carbonyl)-11-methyl-1²,1³-dihydro-1¹H-2,6-diaza-1(2,1)-benzo[d]imidazola-4(1,3)-benzenacycloundecaphane-3,5-dione as a white powder (8 mg, 67%).

¹H NMR (DMSO-d₆) δ: 12.81 (s, 1H), 8.84 (s, 1H), 8.11 (d, 1H), 7.84 (s, 1H), 7.76 (d, 1H), 7.61 (m, 2H), 7.26 (d, 2H), 4.54 (m, 4H), 3.11 (s, 3H), 1.91 (s, 4H), 1.60 (s, 3H), 1.43 (s, 2H); LC/MS: M+H=482.2.

### Example 41: (E)-N-(3-hydroxycyclobutyl)-N,11-dimethyl-3,5-dioxo-1²,1³-dihydro-1¹H-2,6-diaza-1(2,1)-benzo[d]imidazola-4(1,3)-benzenacycloundecaphane-1⁷-carboxamide

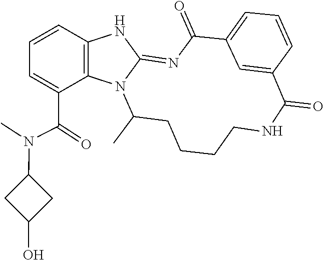 [SMILES: CC1CCCCNC(=O)c2cccc(c2)C(=O)/N=C2\Nc3cccc(C(=O)N(C)C4CC(O)C4)c3N21]

N,N-diisopropylethylamine (21 μl, 123 μmol) was added to a solution of 11-methyl-3,5-dioxo-1¹H-2,6-diaza-1(2,1)-benzo[d]imidazola-4(1,3)-benzenacycloundecaphane-1⁷-carboxylic acid (10 mg, 25 μmol) and HATU (10 mg, 27 μmol) in DMF (0.25 ml) at rt. The solution was stirred for 2 min before 3-(methylamino)cyclobutan-1-ol (6 mg, 49 μmol). The reaction mixture was stirred overnight at RT, diluted with water (1.0 ml) and purified by reverse phase chromatography (C18 column, prep-HPLC, 20-60% MeCN in water containing 0.1% TFA) to give (E)-N-(3-hydroxycyclobutyl)-N,11-dimethyl-3,5-dioxo-1²,1³-dihydro-1¹H-2,6-diaza-1(2,1)-benzo[d]imidazola-4(1,3)-benzenacycloundecaphane-1⁷-carboxamide as a white powder (8 mg, 65%).

¹H NMR (DMSO-d₆) δ: 12.76 (s, 1H), 8.78 (s, 1H), 8.12 (d1H), 7.76 (d, 2H), 7.58 (m, 2H), 7.24 (m, 1H), 6.99 (m, 1H), 5.68-4.06 (m, 2H), 3.98-3.88 (m, 1H), 3.64-3.55 (m, 3H), 3.13-2.96 (m, 3H), 2.80 (s, 1H), 2.31-1.30 (m, 1¹H); LC/MS: M+H=490.3.

### Example 42: (E)-11-methyl-1⁷-(2-oxa-6-azaspiro[3.3]heptane-6-carbonyl)-1²,1³-dihydro-1¹H-2,6-diaza-1(2,1)-benzo[d]imidazola-4(1,3)-benzenacycloundecaphane-3,5-dione

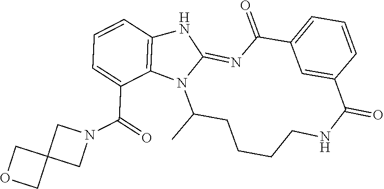 [SMILES: CC1CCCCNC(=O)c2cccc(c2)C(=O)/N=C2\Nc3cccc(C(=O)N4CC5(COC5)C4)c3N21]

N,N-diisopropylethylamine (21 μl, 123 μmol) was added to a solution of 11-methyl-3,5-dioxo-1¹H-2,6-diaza-1(2,1)-benzo[d]imidazola-4(1,3)-benzenacycloundecaphane-1⁷-carboxylic acid (10 mg, 25 μmol) and HATU (10 mg, 27 μmol) in DMF (0.25 ml) at rt. The solution was stirred for 10 min before 2-oxa-6-azaspiro[3.3]heptane oxalate (9 mg, 49 μmol). The reaction mixture was stirred overnight at rt, diluted with water (1.0 ml) and purified by reverse phase chromatography (C18 column, prep-HPLC, 20-60% MeCN in water containing 0.1% TFA) to give (E)-11-methyl-1⁷-(2-oxa-6-azaspiro[3.3]heptane-6-carbonyl)-1²,1³-dihydro-1¹H-2,6-diaza-1(2,1)-benzo[d]imidazola-4(1,3)-benzenacycloundecaphane-3,5-dione as a white powder (2 mg, 17%).

¹H NMR (DMSO-d₆) δ: 12.78 (s, 1H), 8.83 (s, 1H), 8.11 (d, 1H), 7.84 (s, 1H), 7.75 (d, 1H), 7.59 (m, 2H), 7.32-7.04 (m, 2H), 4.68 (m, 5H), 4.35-4.04 (m, 4H), 3.10 (s, 2H), 1.91 (s, 3H), 1.50 (d, 6H); LC/MS: M+H=488.2.

### Example 43: (E)-1⁷-(azetidine-1-carbonyl)-11-methyl-1²,1³-dihydro-1¹H-2,6-diaza-1(2,1)-benzo[d]imidazola-4(1,3)-benzenacycloundecaphane-3,5-dione

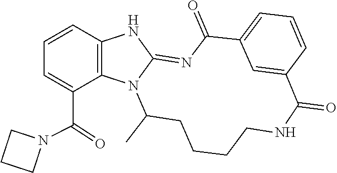 [SMILES: CC1CCCCNC(=O)c2cccc(c2)C(=O)/N=C2\Nc3cccc(C(=O)N4CCC4)c3N21]

N,N-diisopropylethylamine (9 μl, 49 μmol) was added to a solution of 11-methyl-3,5-dioxo-1¹H-2,6-diaza-1(2,1)-benzo[d]imidazola-4(1,3)-benzenacycloundecaphane-1⁷-carboxylic acid (10 mg, 25 μmol) and HATU (10 mg, 27 μmol) in DMF (0.25 ml) at rt. The solution was stirred for 2 min before azetidine (3.3 μl, 49 μmol). The reaction mixture was stirred for 1.5 h at RT, diluted with water (1.0 ml) and purified by reverse phase chromatography (C18 column, prep-HPLC, 20-60% MeCN in water containing 0.1% TFA) to give (E)-1⁷-(azetidine-1-carbonyl)-11-methyl-1²,1³-dihydro-1¹H-2,6-diaza-1(2,1)-benzo[d]imidazola-4(1,3)-benzenacycloundecaphane-3,5-dione as a white powder (7 mg, 67%).

¹H NMR (CDCl₃) δ: 12.04 (s, 1H), 9.05 (s, 1H), 8.28 (d, 1H), 7.95 (d, 1H), 7.61 (m, 1H), 7.39 (d, 1H), 7.21 (m, 2H), 6.91 (s, 1H), 5.15 (s, 1H), 4.30 (m, 2H), 4.06 (m, 2H), 3.72 (s, 1H), 3.33 (s, 1H), 2.53-2.31 (m, 3H), 2.10 (s, 3H), 1.75 (d, 3H), 1.58 (s, 2H); LC/MS: M+H=446.2.

### Example 44: (S,E)-N,N,7-trimethyl-3,5-dioxo-1²,1³-dihydro-1¹H-2,6-diaza-1(2,1)-benzo[d]imidazola-4(1,3)-benzenacycloundecaphane-1⁷-carboxamide

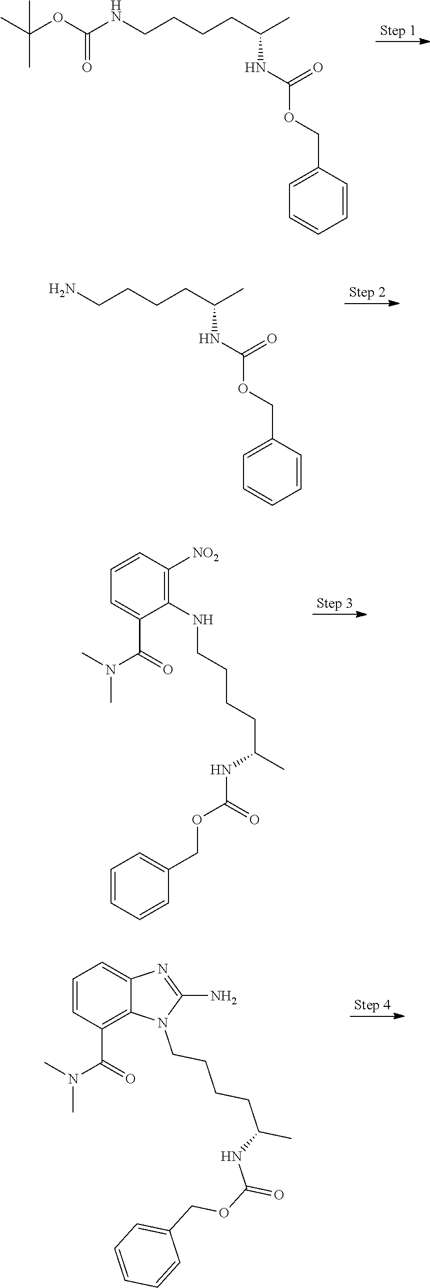 [SMILES: C[C@@H](CCCCNC(=O)OC(C)(C)C)NC(=O)OCc1ccccc1 ; C[C@@H](CCCCN)NC(=O)OCc1ccccc1 ; C[C@@H](CCCCNc1c(C(=O)N(C)C)cccc1[N+](=O)[O-])NC(=O)OCc1ccccc1 ; C[C@@H](CCCCn1c(N)nc2cccc(C(=O)N(C)C)c21)NC(=O)OCc1ccccc1]

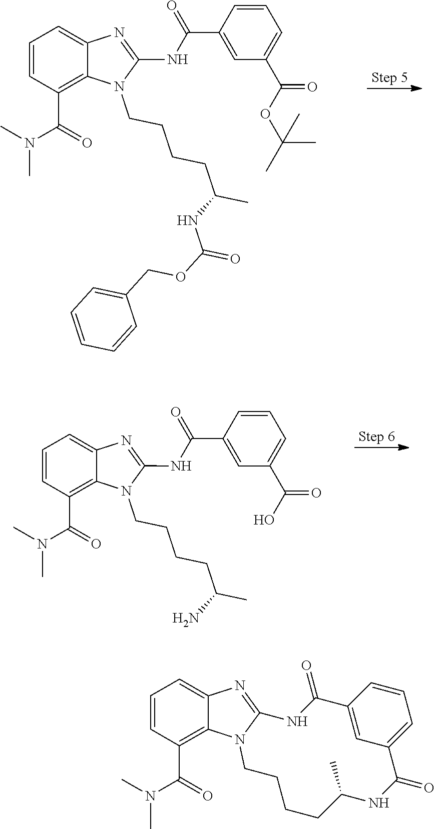

### Step 1: Preparation of (S)-(6-aminohexan-2-yl)carbamate

TFA (2.0 ml, 27.1 mmol) was added to a solution of benzyl tert-butyl hexane-1,5-diyl(S)-dicarbamate (250 mg, 713 μmol), prepared from Z-(D)-lys-boc-OH as described in Mandal et al Journal of Organic Chemistry (2014), 79(17), 8422-8427, in CH₂Cl₂ (2.0 ml). The solution was stirred at rt for 1.5 h. The reaction solution was diluted with CH₂Cl₂ (20 ml) and washed with 1 M NaOH (35 ml). The organic phase was dried and concentrated to give benzyl (S)-(6-aminohexan-2-yl)carbamate (169 mg, 95%)

¹H NMR (DMSO-d₆) δ: 7.35 (m, 5H), 7.10 (d, 1H), 4.99 (s, 2H), 3.48 (m, 1H), 1.30 (m, 7H), 1.05 (d, 3H).

### Step 2: Preparation of benzyl (S)-(6-((2-(dimethylcarbamoyl)-6-nitrophenyl)amino)hexan-2-yl)carbamate

A solution of (S)-(6-aminohexan-2-yl)carbamate (168 mg, 671 μmol), 2-fluoro-N,N-dimethyl-3-nitrobenzamide (142 mg, 671 μmol) and K₂CO₃ (185 mg, 1.34 mmol) in MeCN (15 ml) was stirred at rt overnight. The reaction mixture was filtered and concentrated. The crude product (275 mg) was used without further purification.

### Step 3: Preparation of benzyl (S)-(6-(2-amino-7-(dimethylcarbamoyl)-1H-benzo[d]imidazol-1-yl)hexan-2-yl)carbamate

SnCl₂ (589 mg, 3.11 mmol) and N,N-diisopropylethylamine (1.97 ml, 6.21 mmol) were added to a solution of benzyl (S)-(6-((2-(dimethylcarbamoyl)-6-nitrophenyl)amino)hexan-2-yl)carbamate (275 mg, 621 μmol) in EtOH (20.0 ml). The reaction mixture was stirred for 50 min and concentrated under reduced pressure. The residue was dissolved in EtOAc (30 ml) and 1 M NaOH (30) and filtered through celite. The filtrate was washed with brine and 1 M NaOH and the organic phase was dried and concentrated. The residue was dissolved in MeOH (10 ml) and water (2 ml) and cyanogen bromide (79 mg, 746 μmol) and N,N-diisopropylethylamine (214 μl, 1.24 mmol) were added to the solution. The reaction mixture was stirred for 21 h at rt and the volatiles were removed under reduced pressure. The residue was dissolved in EtOAc (30 ml) and washed with sat. NaHCO₃(aq) (50 ml) and brine (30 ml). The organic phase was dried and concentrated. Purification with flash chromatography (SiO₂, CH₂Cl₂/CH₂Cl₂-MeOH—NH₃(aq) 100:10:1) gave benzyl (S)-(6-(2-amino-7-(dimethylcarbamoyl)-1H-benzo[d]imidazol-1-yl)hexan-2-yl)carbamate as an oil (93 mg, 34%).

¹H NMR (CDCl₃) δ: 7.45 (d, 1H), 7.38 (d, 4H), 7.36-7.31 (m, 1H), 7.12 (m 1H), 6.89 (d, 1H), 5.19-5.03 (m, 2H), 4.86 (s, 2H), 4.62 (s, 1H), 3.79 (s, 2H), 3.17 (s, 3H), 2.93 (s, 3H), 1.69 (d, 4H), 1.55-1.37 (m, 3H), 1.30 (m, 1H), 1.16 (d, 3H).

### Step 4: Preparation of tert-butyl (S)-3-((1-(5-(((benzyloxy)carbonyl)amino)hexyl)-7-(dimethylcarbamoyl)-1H-benzo[d]imidazol-2-yl)carbamoyl)benzoate

N,N-diisopropylethylamine (73 μl, 425 μmol) was added to a solution of 3-(tert-butoxycarbonyl)benzoic acid (52 mg, 234 μmol) and HATU (89 mg, 234 μmol) in MeCN (5.0 ml). The solution was stirred for 3 min before a solution of benzyl (S)-(6-(2-amino-7-(dimethylcarbamoyl)-1H-benzo[d]imidazol-1-yl)hexan-2-yl)carbamate (93 mg, 213 μmol) in MeCN (4.0 ml) was added. The resulting solution was stirred at rt overnight. The reaction mixture was concentrated and purified with flash chromatography (SiO₂, 40-100% EtOAc/pet-ether) to give tert-butyl (S)-3-((1-(5-(((benzyloxy)carbonyl)amino)hexyl)-7-(dimethylcarbamoyl)-1H-benzo[d]imidazol-2-yl)carbamoyl)benzoate (74 mg, 54%).

¹H NMR (CDCl₃) δ: 12.52 (s, 1H), 8.98 (s, 1H), 8.46 (d, 1H), 8.11 (d, 1H), 7.50 (m, 1H), 7.41-7.29 (m, 6H), 7.24 (d, 1H), 7.11 (d, 1H), 5.05 (m, 3H), 4.34 (s, 2H), 3.69 (d, 1H), 3.19 (s, 3H), 2.95 (s, 3H), 1.75 (s, 3H), 1.59 (s, 13H), 1.14 (d, 3H).

### Step 5: Preparation of (S)-3-((1-(5-aminohexyl)-7-(dimethylcarbamoyl)-1H-benzo[d]imidazol-2-yl)carbamoyl)benzoic acid

10% Pd/C (12 mg, 11 μmol) was added to a solution of tert-butyl (S)-3-((1-(5-(((benzyloxy)carbonyl)amino)hexyl)-7-(dimethylcarbamoyl)-1H-benzo[d]imidazol-2-yl)carbamoyl)benzoate (74 mg, 115 μmol) in MeOH (15 ml) and 1M HCl 2.0 ml). The reaction mixture was stirred under a H₂ atmosphere at rt for 40 min. The reaction mixture was filtered through celite and the filtrate was concentrated under reduced pressure. The residue was dissolved in dioxane/5M HCl 1:1 (10 ml) and stirred for 20 h at rt and then concentrated to dryness under reduced pressure. Used in the next step without further purification.

### Step 6: Preparation of (S,E)-N,N,7-trimethyl-3,5-dioxo-1²,1³-dihydro-1¹H-2,6-diaza-1(2,1)-benzo[d]imidazola-4(1,3)-benzenacycloundecaphane-1⁷-carboxamide

A solution of (S)-3-((1-(5-aminohexyl)-7-(dimethylcarbamoyl)-1H-benzo[d]imidazol-2-yl)carbamoyl)benzoic acid (56 mg, 115 μmol) and N,N-diisopropylethylamine (119 μl, 0.69 mmol) in DMF (5 ml) and a solution of HATU (73 mg, 192 μmol) in DMF (5 ml) were simultaneously added through two separate syringes at 250 μl/h over 20 h to a solution of HATU (14 mg, 38 μmol) and N,N-diisopropylethylamine (59 μl, 0.34 mmol) in DMF (5 ml) at rt. The reaction mixture was concentrated to dryness under reduced pressure and purified using chromatography.

¹H NMR (DMSO-d6) δ: 12.71 (s, 1H), 9.00 (s, 1H), 8.10 (d, 1H), 7.73 (d, 2H), 7.64-7.45 (m, 2H), 7.25 (m, 1H), 7.10 (s, 1H), 4.75-4.38 (m, 1H), 4.27-3.85 (m, 1H), 3.72-3.55 (m, 1H), 3.09 (s, 3H), 2.86 (s, 3H), 2.36-1.67 (m, 2H), 1.64-1.32 (m, 4H), 1.03 (d, 3H); LC/MS: M+H=434.2.

### Example 45: (E)-N-(11-methyl-3,5-dioxo-1²,1³-dihydro-1¹H-2,6-diaza-1(2,1)-benzo[d]imidazola-4(1,3)-benzenacycloundecaphane-1⁷-yl)acetamide

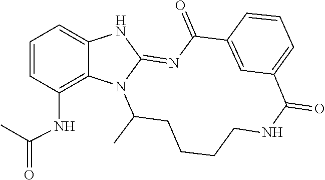 [SMILES: CC(=O)Nc1cccc2c1N1/C(=N/C(=O)c3cccc(c3)C(=O)NCCCCC1C)N2]

Diphenyl phosphoryl azide (17 μl, 81 μmol) was added to a solution of 11-methyl-3,5-dioxo-1¹H-2,6-diaza-1(2,1)-benzo[d]imidazola-4(1,3)-benzenacycloundecaphane-1⁷-carboxylic acid (30 mg, 74 μmol) and TEA (12 μl, 89 μmol) in toluene (1.0 ml). The reaction mixture was stirred for 30 min and NMP (1.0 ml) was added. Stirring was continued for 1.5 h. 1 M HCl (1.0 ml) was added and the reaction mixture was heated at 60° C. overnight. The reaction mixture was cooled and 1 M NaOH (2.0 ml) was added. The mixture was extracted with EtOAc (3×1 ml) and the combined organic layers were dried and concentrated. The residue was purified by reverse phase chromatography C18 column, prep-HPLC, 20-40% MeCN in water containing 0.1% TFA). The purified intermediate was dissolved in THF (3.0 ml) and N,N-diisopropylethylamine (76 μl, 444 μmol) followed by acetyl chloride (16 μl, 222 μmol) was added at RT. The reaction mixture was stirred for 2.5 h and concentrated to dryness. The residue was purified by reverse phase chromatography C18 column, prep-HPLC, 20-40% MeCN in water containing 0.1% TFA) to give (E)-N-(11-methyl-3,5-dioxo-1²,1³-dihydro-1¹H-2,6-diaza-1(2,1)-benzo[d]imidazola-4(1,3)-benzenacycloundecaphane-1⁷-yl)acetamide as a white solid (2.2 mg, 7%).

¹H NMR (MeOH-d₄) δ: 8.98 (s, 1H), 8.21 (d, 1H), 7.82 (d, 1H), 7.61 (m, 1H), 7.46 (d, 1H), 7.25 (m, 1H), 7.03 (d, 1H); LC/MS: M+H=420.0.

### Example 46: (R,E)-N,N,11-trimethyl-3,5-dioxo-1²,1³-dihydro-1¹H-2,6-diaza-1(2,1)-benzo[d]imidazola-4(1,3)-benzenacycloundecaphane-1⁷-sulfonamide

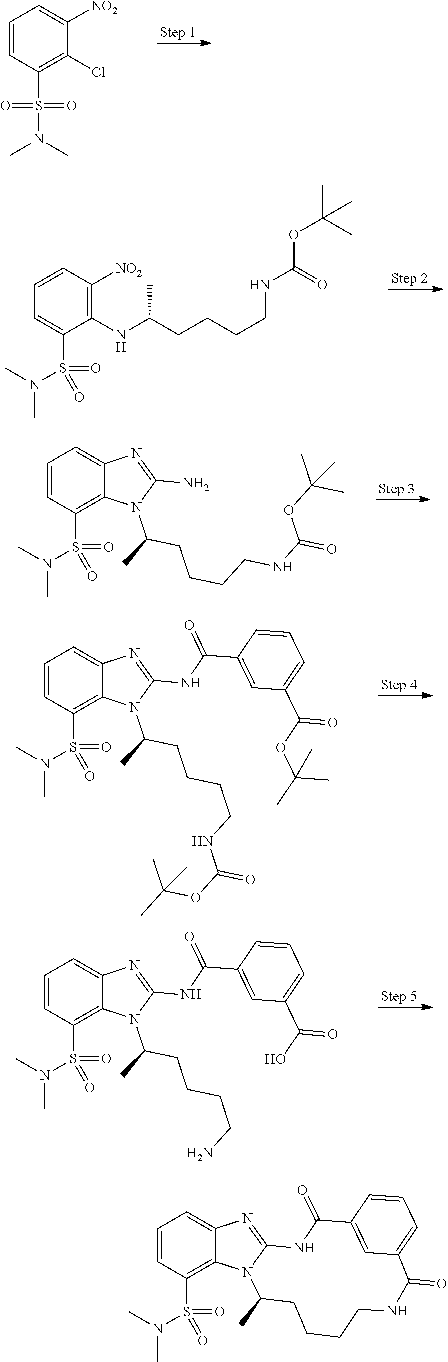

### Step 1: Preparation of tert-butyl (R)-(5-((2-(N,N-dimethylsulfamoyl)-6-nitrophenyl)amino)hexyl)carbamate

Tert-butyl (R)-(5-aminohexyl)carbamate (180 mg, 831 μmol) and K₂CO₃ (313 mg, 2.27 mmol) were added to a solution of 2-chloro-N,N-dimethyl-3-nitrobenzenesulfonamide (200 mg, 756 μmol) in MeCN (5.0 ml). The reaction mixture was stirred overnight at RT. The reaction mixture was further diluted with DMF (2.0 ml) and heated at 60° C. for 48 h. The reaction mixture was filtered and concentrated. The residue was purified by flash chromatography (silica gel, Biotage 12 g, 10-60% EtOAc in petroleum ether) to give tert-butyl (R)-(5-((2-(N,N-dimethylsulfamoyl)-6-nitrophenyl)amino)hexyl)carbamate as an oil (315 mg, 94%).

¹H NMR (DMSO-d6) δ: 12.71 (s, 1H), 9.00 (s, 1H), 8.10 (d, 1H), 7.73 (d, 2H), 7.64-7.45 (m, 2H), 7.25 (m, 1H), 7.10 (s, 1H), 4.75-4.38 (m, 1H), 4.27-3.85 (m, 1H), 3.72-3.55 (m, 1H), 3.09 (s, 3H), 2.86 (s, 3H), 2.36-1.67 (m, 2H), 1.64-1.32 (m, 4H), 1.03 (d, 3H); LC/MS: M+H=445.0.

### Step 2: Preparation of tert-butyl (R)-(5-(2-amino-7-(N,N-dimethylsulfamoyl)-1H-benzo[d]imidazol-1-yl)hexyl)carbamate

A solution of tert-butyl (R)-(5-((2-(N,N-dimethylsulfamoyl)-6-nitrophenyl)amino)hexyl)carbamate (315 mg, 709 μmol) in MeOH (25 ml) was hydrogenated over 10% Pd/C (38 mg, 35 μmol) at rt for 2 h. The reaction mixture was filtered over celite with MeOH and concentrated to give an oil. The residue was dissolved in MeOH (10 ml) and water (2.0 ml) and cyanogen bromide (90 mg, 850 μmol) was added. The resulting solution was stirred at RT overnight. The reaction mixture was concentrated under reduced pressure and purified by flash chromatography (silica gel, 0.5% MeOH in CH₂Cl₂ with 1% NH₃ (28% aq.)) to give tert-butyl (R)-(5-(2-amino-7-(N,N-dimethylsulfamoyl)-1H-benzo[d]imidazol-1-yl)hexyl)carbamate (131 mg, 42%).

LC/MS: M+H=440.0; Rt=1.91 min.

### Step 3: Preparation of tert-butyl (R)-3-((1-(6-((tert-butoxycarbonyl)amino)hexan-2-yl)-7-(N,N-dimethylsulfamoyl)-1H-benzo[d]imidazol-2-yl)carbamoyl)benzoate

A solution of HATU (124 mg, 325 μmol), N,N-diisopropylethylamine (77 μl, 443 μmol) and 3-(tert-butoxycarbonyl)benzoic acid (72 mg, 325 μmol) in CH₂Cl₂ (10 ml) was added to a solution of tert-butyl (R)-(5-(2-amino-7-(N,N-dimethylsulfamoyl)-1H-benzo[d]imidazol-1-yl)hexyl)carbamate (130 mg, 296 μmol) and N,N-diisopropylethylamine (77 μl, 443 μmol) in CH₂Cl₂ (10 ml). The reaction mixture was stirred at RT for 5 days. The reaction mixture was concentrated to dryness and the residues were purified by flash chromatography (silica gel, 25-100% EtOAc in pet. ether) to give butyl (R)-3-((1-(6-((tert-butoxycarbonyl)amino)hexan-2-yl)-7-(N,N-dimethylsulfamoyl)-1H-benzo[d]imidazol-2-yl)carbamoyl)benzoate (151.7 g, 80%).

LC/MS: M+H=644.2; Rt=3.71 min.

### Step 4: Preparation of (R)-3-((1-(6-aminohexan-2-yl)-7-(N,N-dimethylsulfamoyl)-1H-benzo[d]imidazol-2-yl)carbamoyl)benzoic acid

5M HCl (10 ml) was added to a solution of tert-butyl (R)-3-((1-(6-((tert-butoxycarbonyl)amino)hexan-2-yl)-7-(N,N-dimethylsulfamoyl)-1H-benzo[d]imidazol-2-yl)carbamoyl)benzoate (152 mg, 236 μmol) in dioxane (10 ml). The reaction mixture was stirred for 40 h at rt. The reaction mixture was concentrated to dryness under reduced pressure. The product was used without further purification.

LC/MS: M+H=488.0; Rt=1.93 min.

### Step 5: Preparation of (R)—N,N,11-trimethyl-3,5-dioxo-1¹H-2,6-diaza-1(2,1)-benzo[d]imidazola-4(1,3)-benzenacycloundecaphane-1⁷-sulfonamide

HATU (139 mg, 364 μmol) was added to a suspension of (R)-3-((1-(6-aminohexan-2-yl)-7-(N,N-dimethylsulfamoyl)-1H-benzo[d]imidazol-2-yl)carbamoyl)benzoic acid (127 mg, 243 μmol) and N,N-diisopropylethylamine in THF (486 ml). The reaction mixture was heated to 50° C. and stirred overnight. The reaction mixture was concentrated. The residue was dissolved in DMSO and purified by reverse phase chromatography (C18 column, prep-HPLC, 30-60% MeCN in water containing 0.1% formic acid) to give (R,E)-N,N,11-trimethyl-3,5-dioxo-1²,1³-dihydro-1¹H-2,6-diaza-1(2,1)-benzo[d]imidazola-4(1,3)-benzenacycloundecaphane-1⁷-sulfonamide as a white solid (27 mg, 24%).

¹H NMR (DMSO-d6) δ: 13.04 (s, 1H), 8.74 (s, 1H), 8.11 (d, 1H), 7.88-7.71 (m, 3H), 7.67-7.56 (m, 2H), 7.36 (m, 1H), 5.61-5.43 (m, 1H), 3.75-3.56 (m, 1H); LC/MS: M+H=470.0.

### Examples 47 and 48: (E)-N-isopropyl-N,11-dimethyl-3,5-dioxo-1²,1³-dihydro-1¹H-2,6-diaza-1(2,1)-benzo[d]imidazola-4(1,3)-benzenacycloundecaphane-1⁷-carboxamide

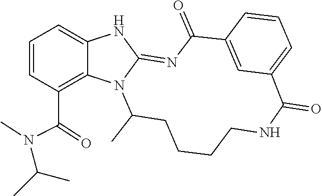 [SMILES: CC(C)N(C)C(=O)c1cccc2c1N1/C(=N/C(=O)c3cccc(c3)C(=O)NCCCCC1C)N2]

N,N-diisopropylethylamine (13 μl, 74 μmol) was added to a solution of 11-methyl-3,5-dioxo-1¹H-2,6-diaza-1(2,1)-benzo[d]imidazola-4(1,3)-benzenacycloundecaphane-1⁷-carboxylic acid (10 mg, 25 μmol) and HATU (9 mg, 25 μmol) in DMF (0.25 ml) at rt. The solution was stirred for 2 min before N-methylpropan-2-amine (8 μl, 74 μmol). The reaction mixture was stirred for 48 h at RT, diluted with water (1.0 ml) and purified by reverse phase chromatography (C18 column, prep-HPLC, 15-45% MeCN in water containing 0.1% TFA) to give (E)-N-isopropyl-N,11-dimethyl-3,5-dioxo-12,13-dihydro-1¹H-2,6-diaza-1(2,1)-benzo[d]imidazola-4(1,3)-benzenacycloundecaphane-1⁷-carboxamide as a white powder (4 mg, 35%).

¹H NMR (DMSO-d6) δ: 8.71 (s, 1H), 8.15 (m, 1H), 7.70 (m, 1H), 7.62 (m, 1H), /0.23 (m, 1H), 7.07 (m, 1H), 4.77 (m, 1H), 4.51 (m, 1H), 1.10 (m, 6H).

The enantiomers were separated on a Daicel IA 25×2 cm SFC column in 3% EtOH/CH₂Cl₂15 ml/min.

### Example 47, Enantiomer 1

Rt=10.25; LC/MS: M+H=446.2.

### Example 48, Enantiomer 2

Rt=16.35; LC/MS: M+H=446.2.

### Examples 49 and 50: (E)-1⁷-(3-methoxy-3-methylazetidine-1-carbonyl)-11-methyl-1²,1³-dihydro-1¹H-2,6-diaza-1(2,1)-benzo[d]imidazola-4(1,3)-benzenacycloundecaphane-3,5-dione

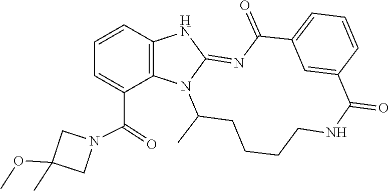 [SMILES: COC1(C)CN(C(=O)c2cccc3c2N2/C(=N/C(=O)c4cccc(c4)C(=O)NCCCCC2C)N3)C1]

N,N-diisopropylethylamine (53 μl, 308 μmol) was added to a solution of 11-methyl-3,5-dioxo-1¹H-2,6-diaza-1(2,1)-benzo[d]imidazola-4(1,3)-benzenacycloundecaphane-1⁷-carboxylic acid (25 mg, 62 μmol) and HATU (26 mg, 68 μmol) in DMF (0.50 ml) at rt. The solution was stirred for 2 min before 3-methoxy-3-methylazetidine (17 mg, 123 μmol). The reaction mixture was stirred for 45 min at RT, diluted with water (1.0 ml) and purified by reverse phase chromatography (C18 column, prep-HPLC, 15-45% MeCN in water containing 0.1% TFA) to give (E)-1⁷-(3-methoxy-3-methylazetidine-1-carbonyl)-11-methyl-12,13-dihydro-1¹H-2,6-diaza-1(2,1)-benzo[d]imidazola-4(1,3)-benzenacycloundecaphane-3,5-dione as a white powder (26 mg, 86%). The enantiomers were separated using chiral chromatography

### Example 49, Enantiomer 1

Chiral separation Daicel IA SFC 2×25 cm in 3% EtOH/CH₂Cl₂ 12 ml/min: Rt=11.27.

¹H NMR (CDCl₃) δ: 11.97 (s, 1H), 9.03 (s, 1H), 8.26 (d1H), 7.95 (d, 1H), 7.59 (m, 1H), 7.37 (d, 1H), 7.23 (m, 1H), 7.18-7.06 (m, 1H), 5.93 (s, 1H), 5.09 (s, 1H), 4.20 (m, 1H), 4.08-3.89 (m, 2H), 3.83-3.62 (m, 2H), 3.26 (d, 4H), 2.05 (s, 3H), 1.73 (d, 4H), 1.53 (d, 4H); LC/MS: M+H=499.0.

### Example 50, Enantiomer 2

Chiral separation Daicel IA SFC 2×25 cm in 3% EtOH/CH₂Cl₂ 12 ml/min: Rt=13.42.

¹H NMR (CDCl₃) δ: 11.94 (s, 1H), 9.03 (s, 1H), 8.26 (d1H), 7.95 (d, 1H), 7.59 (m, 1H), 7.37 (d, 1H), 7.23 (m, 1H), 7.18-7.06 (m, 1H), 5.93 (s, 1H), 5.09 (s, 1H), 4.20 (m, 1H), 4.08-3.89 (m, 2H), 3.83-3.62 (m, 2H), 3.26 (d, 4H), 2.05 (s, 3H), 1.73 (d, 4H), 1.53 (d, 4H); LC/MS: M+H=499.0.

### Examples 51 and 52: (E)-N,N,10-trimethyl-3,5-dioxo-1²,1³-dihydro-1¹H-2,6-diaza-1(2,1)-benzo[d]imidazola-4(1,3)-benzenacycloundecaphane-1⁷-carboxamide

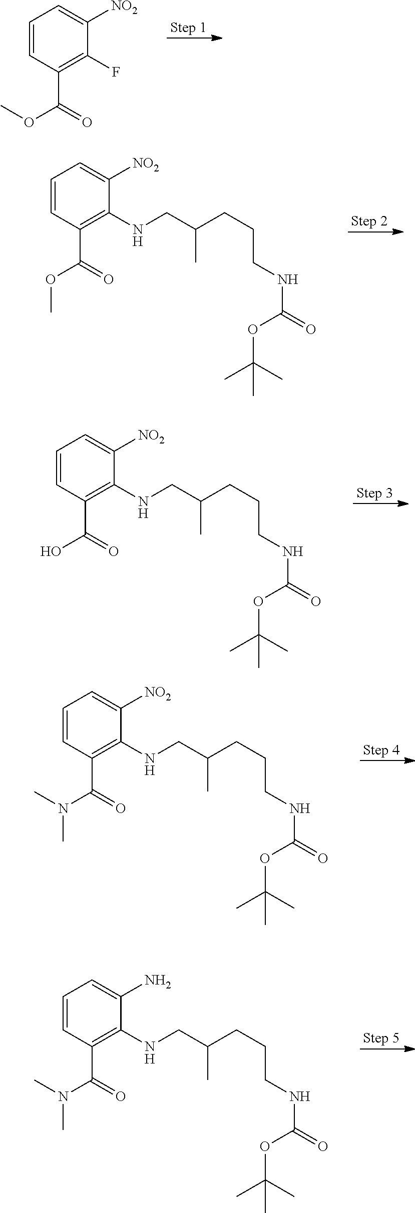

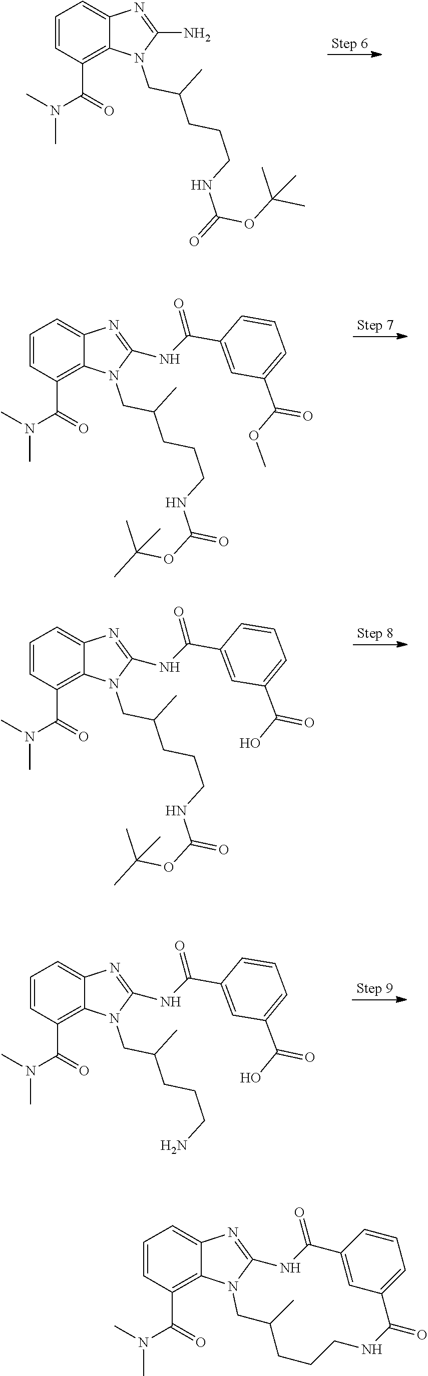

### Step 1: Preparation of methyl 2-((5-((tert-butoxycarbonyl)amino)-2-methylpentyl)amino)-3-nitrobenzoate

N,N-diisopropylethylamine (9.21 ml, 52.7 mmol) was added to a solution of methyl 2-fluoro-3-nitrobenzoate (3.50 g, 17.6 mmol) and tert-butyl N-(5-amino-4-methylpentyl)carbamate (5.23 g, 19.3 mmol) in DMF (30 ml) at rt. The reaction mixture was stirred at 70° C. for 6 h. The reaction was quenched with water and extracted with EtOAc. The organic layer was concentrated under reduced pressure. The product was purified by flash chromatography (SiO₂, 30% EtOAc\Heptane) to give methyl 2-((5-((tert-butoxycarbonyl)amino)-2-methylpentyl)amino)-3-nitrobenzoate (7 g).

LC/MS: M−H=394.20.

### Step 2: Preparation of 2-((5-((tert-butoxycarbonyl)amino)-2-methylpentyl)amino)-3-nitrobenzoic acid

A solution of methyl 2-((5-((tert-butoxycarbonyl)amino)-2-methylpentyl)amino)-3-nitrobenzoate (7 g, 9.91 mmol) and LiOH (2.08 g, 49.6 mmol) in THF (50 ml) and water (50 ml) was stirred at rt for 16 h. The solution was concentrate under reduced pressure. The crude residue was diluted with water (15 ml), cooled to 0° C. and acidified with 1 N HCl. The formed precipitate was filtered, washed with cold water and dried under vacuum to give 2-((5-((tert-butoxycarbonyl)amino)-2-methylpentyl)amino)-3-nitrobenzoic acid (3.78 g, 93%).

LC/MS: M−H=380.15.

### Step 3: Preparation of tert-butyl (5-((2-(dimethylcarbamoyl)-6-nitrophenyl)amino)-4-methylpentyl)carbamate

To a solution of 2-((5-((tert-butoxycarbonyl)amino)-2-methylpentyl)amino)-3-nitrobenzoic acid (7.16 g, 9.23 mmol) in DMF (30 ml) was added N,N-diisopropylethylamine (7.16 g, 554.4 mmol), HATU (14 g, 36.9 mmol) and 2M dimethyl amine (28 ml, 55.4 mmol) at 0° C. The reaction mixture was stirred at rt for 16 h. The reaction mixture was quenched with ice-cold water and extracted with EtOAc. The organic layer was washed with brine solution, dried over Na₂SO₄ and concentrated under reduced pressure. The product was used without further purification.

¹H NMR (DMSO-d6) δ: 8.12 (d, 1H), 8.06 (s, 1H), 7.39 (d, 1H), 6.77 (m, 1H), 2.99 (s, 3H), 2.69 (s, 3H), 1.71 (m, 1H), 0.89 (d, 3H); LC/MS: M+H=409.22.

### Step 4: Preparation of tert-butyl (5-((2-amino-6-(dimethylcarbamoyl)phenyl)amino)-4-methylpentyl)carbamate

To a solution of tert-butyl (5-((2-(dimethylcarbamoyl)-6-nitrophenyl)amino)-4-methylpentyl)carbamate (4.50 g, 7.38 mmol) in MeOH (60 ml) was added nickel (2.17 g, 36.9 mmol). The reaction mixture was hydrogenated at rt under a hydrogen atmosphere for 4 h. The reaction mixture was filtered through celite, washed with EtOAc and concentrated under reduced pressure to get crude. The crude product was purified by column chromatography (silica gel, 50% EtOAc/heptane) to afford tert-butyl (5-((2-amino-6-(dimethylcarbamoyl)phenyl)amino)-4-methylpentyl)carbamate (4 g).

¹H NMR (DMSO-d6) δ: 6.79 (s, 1H), 6.64 (m, 2H), 6.34 (s, 1H), 2.94 (s, 3H), 2.69 (s, 3H), 1.36 (s, 9H); LC/MS: M−H=377.25.

### Step 5: Preparation of tert-butyl (5-(2-amino-7-(dimethylcarbamoyl)-1H-benzo[d]imidazol-1-yl)-4-methylpentyl)carbamate

To a solution of tert-butyl (5-((2-amino-6-(dimethylcarbamoyl)phenyl)amino)-4-methylpentyl)carbamate (4.00 g, 7.4 mmol) in MeOH (20 ml), MeCN (10 ml) and water (10 ml) was added cyanogen bromide (682 mg, 8.14 mmol). The reaction mixture was stirred at 55° C. for 4 h. The reaction mixture was concentrated under reduced pressure, quenched with ice-cold water and extracted with EtOAc. The organic layer was washed with brine solution, dried over Na₂SO₄ and concentrated under reduced pressure to afford the desired product which was used without further purification.

LC/MS: M+H=404.38.

### Step 6: Preparation of methyl 3-((1-(5-((tert-butoxycarbonyl)amino)-2-methylpentyl)-7-(dimethylcarbamoyl)-1H-benzo[d]imidazol-2-yl)carbamoyl)benzoate

To a solution of tert-butyl (5-(2-amino-7-(dimethylcarbamoyl)-1H-benzo[d]imidazol-1-yl)-4-methylpentyl)carbamate (3.20 g, 7.22 mmol) and 3-(methoxycarbonyl)benzoic acid (2.60 g, 14.4 mmol) in DMF (30 ml) was added at 0° C. N,N-diisopropylethylamine (2.80 g, 21.6 mmol) followed by HATU (5.49 g, 14.4 mmol). Reaction was stirred at rt for 16 h. The reaction mixture was quenched with ice-cold water and extracted with EtOAc. The organic layer was washed with brine solution, dried over Na₂SO₄ and concentrated under reduced pressure The crude product was purified by flash chromatography (SiO₂, 48% EtOAc/Heptane) to give methyl 3-((1-(5-((tert-butoxycarbonyl)amino)-2-methylpentyl)-7-(dimethylcarbamoyl)-1H-benzo[d]imidazol-2-yl)carbamoyl)benzoate (3.0 g, 55%).

LC/MS: M+H=566.37.

### Step 7: Preparation of 3-((1-(5-((tert-butoxycarbonyl)amino)-2-methylpentyl)-7-(dimethylcarbamoyl)-1H-benzo[d]imidazol-2-yl)carbamoyl)benzoic acid

To a solution of methyl 3-((1-(5-((tert-butoxycarbonyl)amino)-2-methylpentyl)-7-(dimethylcarbamoyl)-1H-benzo[d]imidazol-2-yl)carbamoyl)benzoate (3.00 g, 3.96 mmol) in THF (30 ml) and water (30 ml) was added LiOH (831 mg, 198.8 mmol). The reaction mixture was stirred at 50° C. for 3 h. The reaction mixture was diluted with water and extracted with EtOAc. The aqueous phase was acidified with 3N HCl and extracted with EtOAc. The organic layer was concentrated under deduced pressure and the product was used without further purification.

¹H NMR (DMSO-d6) δ: 8.79 (s, 1H), 8.45 (d, 1H), 8.07 (d, 1H), 7.63 (m, 2H), 7.28 (m, 1H), 7.15 (d, 1H), 4.14 (m, 2H), 3.16 (s, 3H), 2.88 (s, 3H), 1.33 (s, 9H), 0.78 (m, 3H); LC/MS: M+H=552.35.

### Step 8: Preparation of 3-((1-(5-amino-2-methylpentyl)-7-(dimethylcarbamoyl)-1H-benzo[d]imidazol-2-yl)carbamoyl)benzoic acid

To a solution of 3-((1-(5-((tert-butoxycarbonyl)amino)-2-methylpentyl)-7-(dimethylcarbamoyl)-1H-benzo[d]imidazol-2-yl)carbamoyl)benzoic acid (500 mg, 798 μmol) in CH₂Cl₂ (5.0 ml) was added 4 M HCl (1.99 ml, 8 mmol) at 0° C. The solution was stirred at rt for 2 h. The reaction mixture was concentrated under reduced pressure and the product was used without further purification.

¹H NMR (DMSO-d6) δ: 8.79 (s, 1H), 8.45 (d, 1H), 8.09 (d, 1H), 7.63 (m, 2H), 7.28 (m, 1H), 7.15 (d, 1H), 4.10 (m, 2H), 3.11 (s, 3H), 2.88 (s, 3H), 2.69 (m, 2H), 0.88 (m, 3H); LC/MS: M+H=452.26.

### Step 9: Preparation of N,N,10-trimethyl-3,5-dioxo-1¹H-2,6-diaza-1(2,1)-benzo[d]imidazola-4(1,3)-benzenacycloundecaphane-1⁷-carboxamide

To a solution of 3-((1-(5-amino-2-methylpentyl)-7-(dimethylcarbamoyl)-1H-benzo[d]imidazol-2-yl)carbamoyl)benzoic acid (300 mg, 551 μmol) in MeCN (200 ml) and DMF (30 ml) was added TCFH (233 mg, 831 μmol) and 1-methyl-1H-imidazole (185 μl, 2.33 mmol) at 0° C. The reaction mixture was stirred at rt for 2 h. The reaction mixture was concentrated under reduced pressure. The residue was triturated with water and the precipitate was collected by filtration. The crude product was purified by preparative HPLC followed by separation of the enantiomers by chiral chromatography to give enantiomer 1 (20 mg) and enantiomer 2 (24 mg).

### Example 51, Enantiomer 1

¹H NMR (DMSO-d₆) δ: 12.79 (s, 1H), 8.95 (s, 1H), 8.10 (d, 1H), 7.87 (m, 1H), 7.75 (d, 1H), 7.58 (m, 2H), 7.25 (m, 1H), 3.10 (s, 3H), 2.18 (m, 1H); LC/MS: M+H=434.45.

ChiralPak IG (4.6×250) 5 um, IPA/Ethyl Acetate=50:50 (v/v), Flow Rate: 1 ml/min, Rt=6.55 min

### Example 52, Enantiomer 2

¹H NMR (DMSO-d₆) δ: 12.79 (s, 1H), 8.95 (s, 1H), 8.10 (d, 1H), 7.87 (m, 1H), 7.75 (d, 1H), 7.58 (m, 2H), 7.25 (m, 1H), 3.10 (s, 3H), 2.18 (m, 1H); LC/MS: M+H=434.45.

ChiralPak IG (4.6×250) 5 um, IPA/Ethyl Acetate=50:50 (v/v), Flow Rate: 1 ml/min, Rt=10.37 min

### Examples 53 and 54: (S,E)-1⁶-chloro-N,N,11-trimethyl-3,5-dioxo-1²,1³-dihydro-1¹H-2,6-diaza-1(2,1)-benzo[d]imidazola-4(1,3)-benzenacycloundecaphane-1⁷-carboxamide

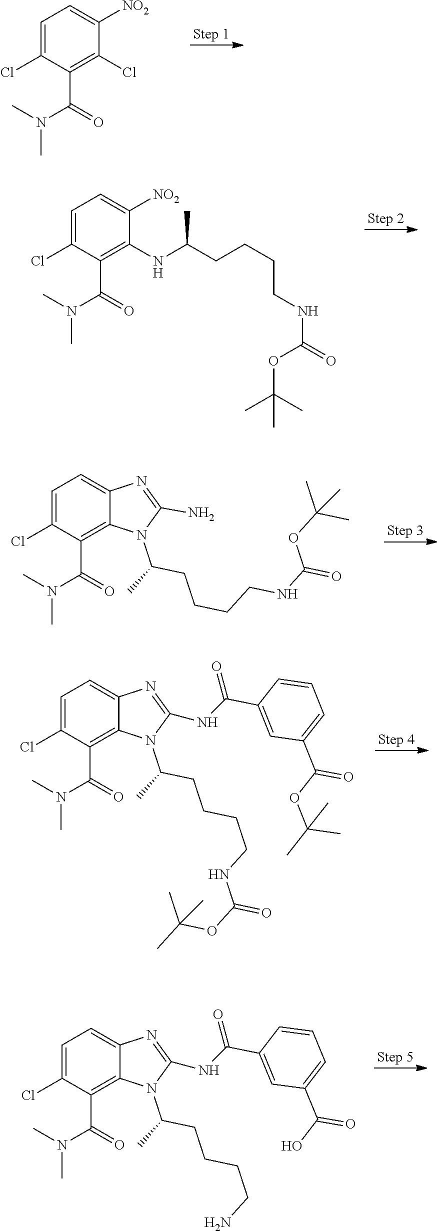 [SMILES: CN(C)C(=O)c1c(Cl)ccc([N+](=O)[O-])c1Cl ; C[C@@H](CCCCNC(=O)OC(C)(C)C)Nc1c([N+](=O)[O-])ccc(Cl)c1C(=O)N(C)C ; C[C@@H](CCCCNC(=O)OC(C)(C)C)n1c(N)nc2ccc(Cl)c(C(=O)N(C)C)c21 ; C[C@@H](CCCCNC(=O)OC(C)(C)C)n1c(NC(=O)c2cccc(C(=O)OC(C)(C)C)c2)nc2ccc(Cl)c(C(=O)N(C)C)c21 ; C[C@@H](CCCCN)n1c(NC(=O)c2cccc(C(=O)O)c2)nc2ccc(Cl)c(C(=O)N(C)C)c21]

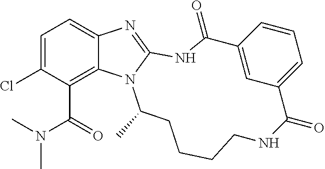 [SMILES: C[C@H]1CCCCNC(=O)c2cccc(c2)C(=O)Nc2nc3ccc(Cl)c(C(=O)N(C)C)c3n21]

### Step 1: Preparation of tert-butyl (S)-(5-((3-chloro-2-(dimethylcarbamoyl)-6-nitrophenyl)amino)hexyl)carbamate

2,6-dichloro-N,N-dimethyl-3-nitrobenzamide (281 mg, 1.07 mmol) was added to a solution of tert-butyl (S)-(5-aminohexyl)carbamate (231 mg, 1.07 mmol) in MeCN (5.0 ml) at rt. K₂CO₃ (443 mg, 3.20 mmol) was added and the suspension heated to 40° C. The reaction mixture was stirred for 6 days. The reaction mixture was cooled to rt, filtered and concentrated to dryness. The residue was dissolved in MeCN and water and purified by reverse phase chromatography on a C18 flash cartridge, 20-70% MeCN in water with 0.1% TFA to give tert-butyl (S)-(5-((3-chloro-2-(dimethylcarbamoyl)-6-nitrophenyl)amino)hexyl)carbamate (348 mg, 73%). LC/MS: M+Na=465.0. Rt=2.93.

### Step 2: Preparation of tert-butyl (S)-(5-(2-amino-6-chloro-7-(dimethylcarbamoyl)-1H-benzo[d]imidazol-1-yl)hexyl)carbamate

To a solution of (S)-(5-((3-chloro-2-(dimethylcarbamoyl)-6-nitrophenyl)amino)hexyl)carbamate (348 mg, 788 μmol) in MeOH (15 ml) was added 10% Pd/C (42 mg, 39 μmol) and the resulting mixture was hydrogenated at rt for 1.5 h. The reaction mixture was filtered through celite with MeOH and the filtrate was concentrated under reduced pressure. The residue was dissolved in water (1.0 ml) and MeOH (5.0 ml) and cyanogen bromide (100 mg, 945 μmol) was added. The reaction mixture was stirred at rt overnight. The reaction mixture was concentrated under reduced pressure and purified by flash chromatography on silica gel, 1-7% MeOH in CH₂Cl₂ with 1% NH₃ (28% aq.) to give the product as a yellow oil (238.7 mg, 69%). The material was used without further purification.

LC/MS: M+H=438.0. Rt=1.97.

### Step 3: Preparation of tert-butyl (S)-3-((1-(6-((tert-butoxycarbonyl)amino)hexan-2-yl)-6-chloro-7-(dimethylcarbamoyl)-1H-benzo[d]imidazol-2-yl)carbamoyl)benzoate

To a solution of tert-butyl (S)-(5-(2-amino-6-chloro-7-(dimethylcarbamoyl)-1H-benzo[d]imidazol-1-yl)hexyl)carbamate (239 mg, 545 μmol) and N,N-diisopropylethylamine (282 μl, 1.64 mmol) in MeCN (5 ml) was added a solution of HATU (249 mg, 654 μmol) in MeCN (5 ml). To the resulting solution was added 3-(tert-butoxycarbonyl)benzoic acid (145 mg, 654 μmol). The reaction mixture was tired over night at rt. The reaction mixture was concentrated under reduced pressure and purified by flash chromatography on silica gel using 0-50% EtOAc in pet. Ether to give tert-butyl (S)-3-((1-(6-((tert-butoxycarbonyl)amino)hexan-2-yl)-6-chloro-7-(dimethylcarbamoyl)-1H-benzo[d]imidazol-2-yl)carbamoyl)benzoate (141 mg, 40%)

LC/MS: M+H=642.3. Rt=3.57.

### Step 4: Preparation of tert-butyl (S)-3-((1-(6-((tert-butoxycarbonyl)amino)hexan-2-yl)-6-chloro-7-(dimethylcarbamoyl)-1H-benzo[d]imidazol-2-yl)carbamoyl)benzoate

To a solution of tert-butyl (S)-3-((1-(6-((tert-butoxycarbonyl)amino)hexan-2-yl)-6-chloro-7-(dimethylcarbamoyl)-1H-benzo[d]imidazol-2-yl)carbamoyl)benzoate (142 mg, 220 μmol) in dioxane (10 ml) was added 5M HCl (aq) (10 ml). The solution was stirred at rt overnight. The solution was concentrated and the product was used without further purification.

LC/MS: M+H=486.2. Rt=1.80.

### Step 5: Preparation of (S,E)-1⁶-chloro-N,N,11-trimethyl-3,5-dioxo-1²,1³-dihydro-1¹H-2,6-diaza-1(2,1)-benzo[d]imidazola-4(1,3)-benzenacycloundecaphane-1⁷-carboxamide

HATU (126 mg, 330 μmol) was added to a solution of tert-butyl (S)-3-((1-(6-((tert-butoxycarbonyl)amino)hexan-2-yl)-6-chloro-7-(dimethylcarbamoyl)-1H-benzo[d]imidazol-2-yl)carbamoyl)benzoate (115 mg, 220 μmol) and N,N-diisopropylethylamine (190 μl, 1.10 mmol) in THF (440 ml). The reaction mixture was heated to 60° C. and stirred overnight. The mixture was concentrated to dryness under reduced pressure. The residue was dissolved in DMSO and purified by reverse phase (C18 column, isocratic elution at 30% MeCN in water containing 0.1% formic acid over 20 min). Fractions containing the first eluting product peak (P1) were collected separately and freeze-dried to give a white solid (6.52 mg, 6.3%). Fractions containing the second eluting product peak (Example 54, P2) were collected separately and freeze-dried to white solids (7.35 mg, 7.1%).

### Example 53, Atropisomer/Stereoisomer 1

¹H NMR (DMSO-d₆) δ: 12.82 (s, 1H), 8.73 (s, 1H), 8.11 (d, 1H), 7.85-7.68 (m, 2H), 7.60 (m, 1H), 7.51 (d, 1H), 7.34 (d, 1H), 4.49-4.19 (m, 1H), 3.10 (s, 3H), 2.88 (s, 3H), 1.99-1.88 (m, 1H), 1.84-1.74 (m, 1H), 1.74-1.63 (m, 3H), 1.63-1.48 (m, 2H), 1.47-1.35 (m, 1H); LC/MS: M+H=468.0.

### Example 54, Atropisomer/Stereoisomer 2

¹H NMR (DMSO-d₆) δ: 12.75 (s, 1H), 8.71 (s, 1H), 8.11 (d, 1H), 7.82-7.71 (m, 2H), 7.59 (t, 1H), 7.52 (d, 1H), 7.35 (d, 1H), 4.41-4.27 (m, 1H), 3.09 (s, 3H), 2.82 (s, 3H), 1.99-1.86 (m, 1H), 1.85-1.74 (m, 1H), 1.70-1.62 (m, 3H), 1.62-1.51 (m, 2H), 1.48-1.38 (m, 1H); LC/MS: M+H=468.0.

### Examples 55 and 56: (R,E)-1⁶-chloro-N,N,11-trimethyl-3,5-dioxo-1²,1³-dihydro-1¹H-2,6-diaza-1(2,1)-benzo[d]imidazola-4(1,3)-benzenacycloundecaphane-1⁷-carboxamide

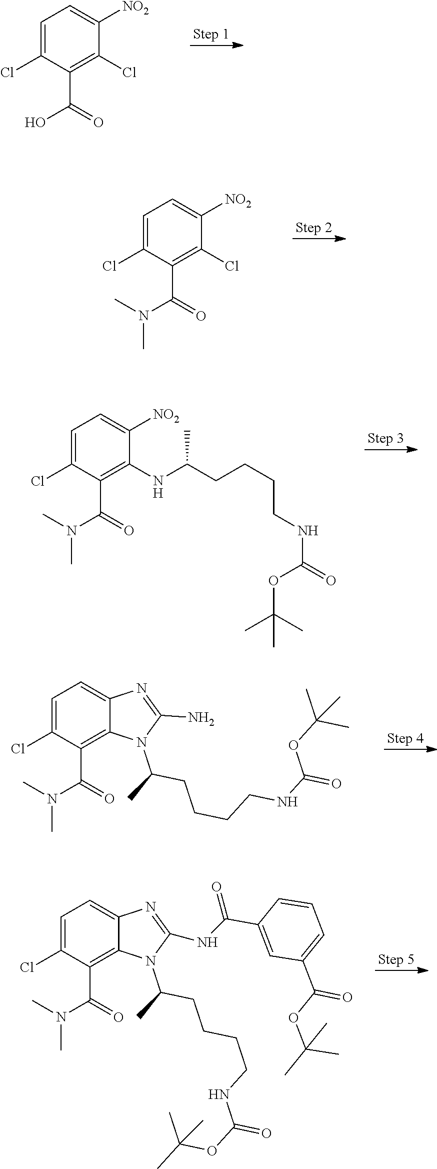 [SMILES: O=C(O)c1c(Cl)ccc([N+](=O)[O-])c1Cl ; CN(C)C(=O)c1c(Cl)ccc([N+](=O)[O-])c1Cl ; C[C@H](CCCCNC(=O)OC(C)(C)C)Nc1c([N+](=O)[O-])ccc(Cl)c1C(=O)N(C)C ; C[C@H](CCCCNC(=O)OC(C)(C)C)n1c(N)nc2ccc(Cl)c(C(=O)N(C)C)c21 ; C[C@H](CCCCNC(=O)OC(C)(C)C)n1c(NC(=O)c2cccc(C(=O)OC(C)(C)C)c2)nc2ccc(Cl)c(C(=O)N(C)C)c21]

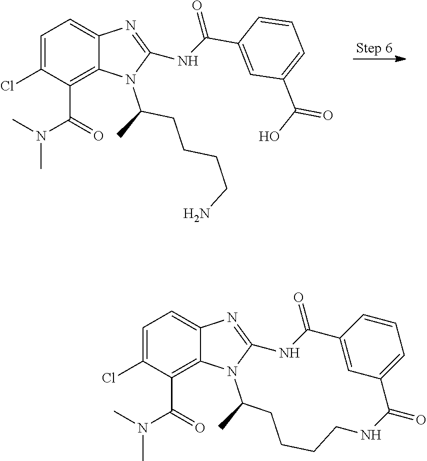 [SMILES: C[C@H](CCCCN)n1c(NC(=O)c2cccc(C(=O)O)c2)nc2ccc(Cl)c(C(=O)N(C)C)c21 ; C[C@@H]1CCCCNC(=O)c2cccc(c2)C(=O)Nc2nc3ccc(Cl)c(C(=O)N(C)C)c3n21]

### Step 1: Preparation 2,6-dichloro-N,N-dimethyl-3-nitrobenzamide

A solution of 2,6-dichloro-3-nitrobenzoic acid (1.0 g, 4.24 mmol) in thionyl chloride (4.99 ml, 68.6 mmol) was heated at 80° C. for 2 h. The reaction mixture was cooled to rt and concentrated to dryness through repeated evaporation with toluene. The residue was dissolved in THF (3.5 ml), cooled to 0° C. and dimethyl amine (2 M in THF, 4.81 ml, 9.62 mmol) was added dropwise. The mixture was stirred for 1 h. The reaction mixture was diluted with EtOAc and washed with water. The organic phase was dried and concentrated to dryness. The product was used without further purification.

¹H NMR (chloroform, d₃) δ: 7.83 (d, 1H), 7.49 (d, 1H), 3.19 (s, 3H), 2.90 (s, 3H); M+H=265.0.

### Step 2: Preparation of tert-butyl (R)-(5-((3-chloro-2-(dimethylcarbamoyl)-6-nitrophenyl)amino)hexyl)carbamate

2,6-dichloro-N,N-dimethyl-3-nitrobenzamide (281 mg, 1.07 mmol) was added to a solution of tert-butyl (R)-(5-aminohexyl)carbamate (231 mg, 1.07 mmol) in MeCN (5.0 ml) at rt. K₂CO₃ (443 mg, 3.20 mmol) was added and the suspension heated to 40° C. The reaction mixture was stirred for 6 days. The reaction mixture was cooled to rt, filtered and concentrated to dryness. The residue was dissolved in MeCN and water and purified by reverse phase chromatography on a C18 flash cartridge, 20-70% MeCN in water with 0.1% TFA to give tert-butyl (R)-(5-((3-chloro-2-(dimethylcarbamoyl)-6-nitrophenyl)amino)hexyl)carbamate (356 mg, 75%).

LC/MS: M+Na=465.0. Rt=2.93.

### Step 3: Preparation of tert-butyl (R)-(5-(2-amino-6-chloro-7-(dimethylcarbamoyl)-1H-benzo[d]imidazol-1-yl)hexyl)carbamate

To a solution of (R)-(5-((3-chloro-2-(dimethylcarbamoyl)-6-nitrophenyl)amino)hexyl)carbamate (354 mg, 799 μmol) in MeOH (15 ml) was added 10% Pd/C (43 mg, 40 μmol) and the resulting mixture was hydrogenated at rt for 1.5 h. The reaction mixture was filtered through celite with MeOH and the filtrate was concentrated under reduced pressure. The residue was dissolved in water (1.0 ml) and MeOH (5.0 ml) and cyanogen bromide (102 mg, 959 μmol) was added. The reaction mixture was stirred at rt overnight. The reaction mixture was concentrated under reduced pressure and purified by flash chromatography on silica gel, 1-7% MeOH in CH₂Cl₂ with 1% NH₃ (28% aq.) to give the product as a yellow oil (247 mg, 71%). The material was used without further purification.

LC/MS: M+H=438.0. Rt=1.97.

### Step 4: Preparation of tert-butyl (R)-3-((1-(6-((tert-butoxycarbonyl)amino)hexan-2-yl)-6-chloro-7-(dimethylcarbamoyl)-1H-benzo[d]imidazol-2-yl)carbamoyl)benzoate

To a solution of tert-butyl (R)-(5-(2-amino-6-chloro-7-(dimethylcarbamoyl)-1H-benzo[d]imidazol-1-yl)hexyl)carbamate (247 mg, 563 μmol) and N,N-diisopropylethylamine (291 μl, 1.69 mmol) in MeCN (5 ml) was added a solution of HATU (257 mg, 676 μmol) in MeCN (5 ml). To the resulting solution was added 3-(tert-butoxycarbonyl)benzoic acid (150 mg, 676 μmol). The reaction mixture was tired over night at rt. The reaction mixture was concentrated under reduced pressure and purified by flash chromatography on silica gel using 0-50% EtOAc in pet. Ether to give tert-butyl (R)-3-((1-(6-((tert-butoxycarbonyl)amino)hexan-2-yl)-6-chloro-7-(dimethylcarbamoyl)-1H-benzo[d]imidazol-2-yl)carbamoyl)benzoate (257 mg, 71%)

LC/MS: M+H=642.3. Rt=3.57.

### Step 5: Preparation of tert-butyl (R)-3-((1-(6-((tert-butoxycarbonyl)amino)hexan-2-yl)-6-chloro-7-(dimethylcarbamoyl)-1H-benzo[d]imidazol-2-yl)carbamoyl)benzoate

To a solution of tert-butyl (R)-3-((1-(6-((tert-butoxycarbonyl)amino)hexan-2-yl)-6-chloro-7-(dimethylcarbamoyl)-1H-benzo[d]imidazol-2-yl)carbamoyl)benzoate (257 mg, 400 μmol) in dioxane (10 ml) was added 5M (HCl (aq) (10 ml). The solution was stirred at rt overnight. The solution was concentrated and the product was used without further purification.

LC/MS: M+H=486.2. Rt=1.80.

### Step 6: Preparation of (R,E)-1⁶-chloro-N,N,11-trimethyl-3,5-dioxo-1²,1³-dihydro-1¹H-2,6-diaza-1(2,1)-benzo[d]imidazola-4(1,3)-benzenacycloundecaphane-1⁷-carboxamide

HATU (228 mg, 600 μmol) was added to a solution of tert-butyl (R)-3-((1-(6-((tert-butoxycarbonyl)amino)hexan-2-yl)-6-chloro-7-(dimethylcarbamoyl)-1H-benzo[d]imidazol-2-yl)carbamoyl)benzoate (209 mg, 400 μmol) and N,N-diisopropylethylamine (345 μl, 2.0 mmol) in THF (800 ml). The reaction mixture was heated to 60° C. and stirred overnight. The mixture was concentrated to dryness under reduced pressure. The residue was dissolved in DMSO and purified by reverse phase (C18 column, isocratic elution at 30% MeCN in water containing 0.1% formic acid over 20 min). Fractions containing the first eluting product peak (example 55, P1) were collected separately and freeze-dried to give a white solid (9.8 mg, 5.3%). Fractions containing the second eluting product peak (Example 56, P2) were collected separately and freeze-dried to white solids (10.6 mg, 5.7%).

### Example 55, Atropisomer 1

¹H NMR (DMSO-d₆) δ: 12.83 (s, 1H), 8.73 (s, 1H), 8.11 (d, 1H), 7.76 (d, 2H), 7.59 (m, 1H), 7.52 (d, 1H), 7.34 (d, 1H), 4.47-4.18 (m, 1H), 3.71-3.54 (m, 1H), 3.10 (s, 3H), 3.02-2.93 (m, 1H), 2.88 (s, 3H), 2.00-1.85 (m, 1H), 1.86-1.74 (m, 1H), 1.69 (s, 3H), 1.62-1.47 (m, 2H), 1.48-1.34 (m, 1H); LC/MS: M+H=468.0.

### Example 56, Atropisomer 2

¹H NMR (DMSO-d₆) δ: 12.82 (s, 1H), 8.71 (s, 1H), 8.11 (d, 1H), 7.85-7.69 (m, 2H), 7.59 (m, 1H), 7.52 (d, 1H), 7.34 (d, 1H), 4.39-4.27 (m, 1H), 3.70-3.55 (m, 1H), 3.09 (s, 3H), 3.02-2.93 (m, 1H), 2.83 (s, 3H), 2.00-1.87 (m, 1H), 1.84-1.73 (m, 1H), 1.65 (s, 3H), 1.60-1.51 (m, 2H), 1.49-1.37 (m, 1H); LC/MS: M+H=468.0.

### Example 57: (R,E)-1⁷-(4,5-dimethyl-4H-1,2,4-triazol-3-yl)-11-methyl-1²,1³-dihydro-1¹H-2,6-diaza-1(2,1)-benzo[d]imidazola-4(1,3)-benzenacycloundecaphane-3,5-dione

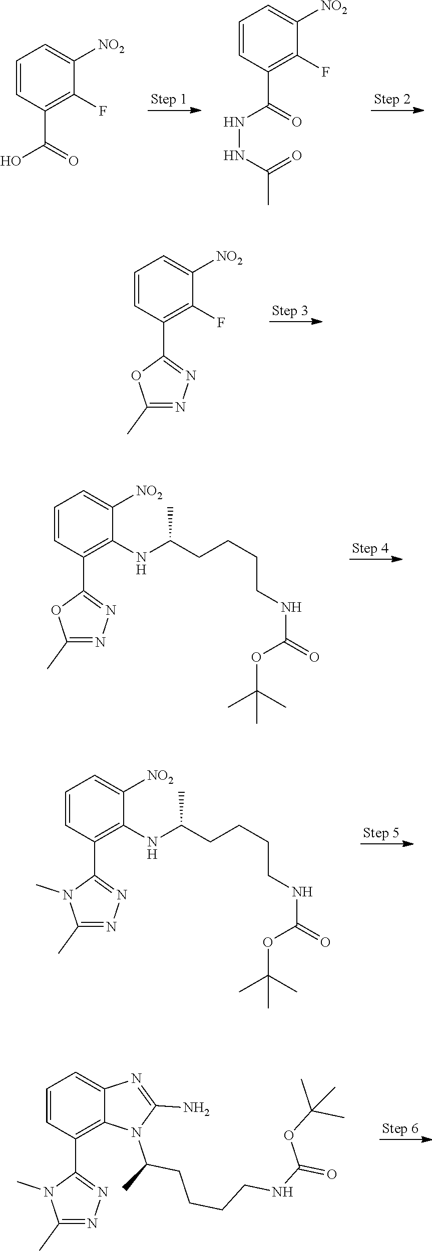 [SMILES: O=C(O)c1cccc([N+](=O)[O-])c1F ; CC(=O)NNC(=O)c1cccc([N+](=O)[O-])c1F ; Cc1nnc(-c2cccc([N+](=O)[O-])c2F)o1 ; Cc1nnc(-c2cccc([N+](=O)[O-])c2N[C@H](C)CCCCNC(=O)OC(C)(C)C)o1 ; Cc1nnc(-c2cccc([N+](=O)[O-])c2N[C@H](C)CCCCNC(=O)OC(C)(C)C)n1C ; Cc1nnc(-c2cccc3nc(N)n([C@H](C)CCCCNC(=O)OC(C)(C)C)c23)n1C]

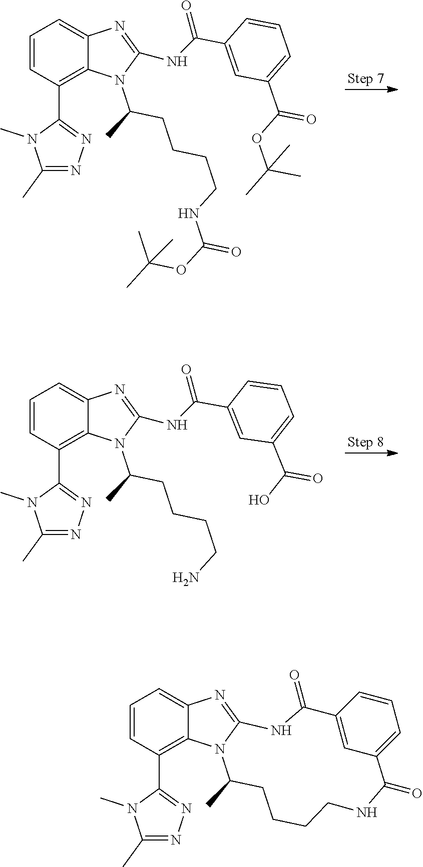 [SMILES: Cc1nnc(-c2cccc3nc(NC(=O)c4cccc(C(=O)OC(C)(C)C)c4)n([C@H](C)CCCCNC(=O)OC(C)(C)C)c23)n1C ; Cc1nnc(-c2cccc3nc(NC(=O)c4cccc(C(=O)O)c4)n([C@H](C)CCCCN)c23)n1C ; Cc1nnc(-c2cccc3nc4n(c23)[C@H](C)CCCCNC(=O)c2cccc(c2)C(=O)N4)n1C]

### Step 1: Preparation of N′-acetyl-2-fluoro-3-nitrobenzohydrazide

A solution of acetohydrazide (91 mg, 1.23 mmol) in MeCN (0.5 ml) with a few drops of DMF was added to a solution of 2-fluoro-3-nitrobenzoyl chloride (250 mg, 1.23 mmol) in MeCN (10 ml). N,N-diisopropylethylamine (212 μl, 1.23 mmol) was added to the resulting reaction mixture. The reaction mixture was stirred at rt for 1 h and then concentrated to dryness. The residue was diluted with EtOAc and NaHCO₃ (aq). The phases were separated, and the aqueous layer was acidified to pH 2-3 with H₂SO₄ and extracted twice with EtOAc. The combined organic phases were washed with brine, dried and concentrated to give N′-acetyl-2-fluoro-3-nitrobenzohydrazide as a solid (237 mg, 80%).

¹H NMR (DMSO-d₆) δ: 8.28 (m, 1H), 7.91 (m, 1H), 7.52 (m, 1H), 1.90 (m, 1H).

### Step 2: Preparation of 2-(2-fluoro-3-nitrophenyl)-5-methyl-1,3,4-oxadiazole

POCl₃ (116 μl, 1.24 mmol) was added to a solution of N′-acetyl-2-fluoro-3-nitrobenzohydrazide (100 mg, 415 μmol) in toluene (5.0 ml). The reaction mixture was heated at 120° C. for 30 min. The reaction mixture was cooled to rt and diluted with EtOAc and washed with NaHCO₃ and brine. The organic phase was concentrated to dryness under reduced pressure to give 2-(2-fluoro-3-nitrophenyl)-5-methyl-1,3,4-oxadiazole (83 mg, 90%) that was used without further purification.

¹H NMR (DMSO-d₆) δ: 8.37 (m, 2H), 7.64 (m, 1H), 2.62 (m, 1H).

### Step 3: Preparation of tert-butyl (R)-(5-((2-(5-methyl-1,3,4-oxadiazol-2-yl)-6-nitrophenyl)amino)hexyl)carbamate

A mixture of 2-(2-fluoro-3-nitrophenyl)-5-methyl-1,3,4-oxadiazole (81 mg, 363 μmol), tert-butyl (R)-(5-aminohexyl)carbamate (78 mg, 363 μmol) and K₂CO₃ (100 mg, 726 μmol) in MeCN (10 ml) was stirred overnight at rt. The reaction mixture was filtered and concentrated to dryness and used in the next step without further purification.

¹H NMR (CDCl₃) δ: 8.05 (m, 1H), 7.98 (m, 1H, 7.82 (m, 1H), 6.80 (m, 1H), 4.50 (m, 1H), 3.30 (m, 1H), 3.05 (m, 2H), 2.70 (s, 3H, 1.18 (d, 3H).

### Step 4: Preparation of tert-butyl (R)-(5-((2-(4,5-dimethyl-4H-1,2,4-triazol-3-yl)-6-nitrophenyl)amino)hexyl)carbamate

Acetic acid (497 μl, 8.70 mmol) was added to a solution of tert-butyl (R)-(5-((2-(5-methyl-1,3,4-oxadiazol-2-yl)-6-nitrophenyl)amino)hexyl)carbamate (152 mg, 362 pmol) in methylamine 40% in MeOH (3.70 ml) in a vial. The vial was sealed, and the solution was heated in a microwave at 140° C. for 1 h. The volatiles were removed under reduced pressure and the residue was diluted with water and extracted with EtOAc and CH₂Cl₂. The combined organic phases were washed with brine, NaHCO₃. The organic phase was evaporated to dryness and purified using reverse phase chromatography (Gemini NX-C18, 25-55% MeCN/H₂O(TFA)) to give tert-butyl (R)-(5-((2-(4,5-dimethyl-4H-1,2,4-triazol-3-yl)-6-nitrophenyl)amino)hexyl)carbamate (50 mg, 32 mg).

¹H NMR (CDCl₃) δ: 8.39 (m, 1H), 7.63 (m, 1H), 7.06 (m, 1H), 3.55 (s, 3H), 3.04 (m, 2H), 2.76 (s, 3H), 2.40 (m, 1H), 1.54-1.00 (m, 17H), 0.96 (d, 3H).

### Step 5: Preparation of tert-butyl (R)-(5-(2-amino-7-(4,5-dimethyl-4H-1,2,4-triazol-3-yl)-1H-benzo[d]imidazol-1-yl)hexyl)carbamate

A solution of tert-butyl (R)-(5-((2-(4,5-dimethyl-4H-1,2,4-triazol-3-yl)-6-nitrophenyl)amino)hexyl)carbamate (50 mg, 116 μmol) in MeOH (25 ml) was hydrogenated over 5% Pd/C (12 mg, 6 μmol) at rt for 40 min. The reaction mixture was filtered into a solution of cyanogen bromide (15 mg, 139 μmol) in MeOH (5.0 ml). The resulting solution was stirred at rt for 1.5 h and at 40° C. for 24 h. The reaction mixture was concentrated under reduced pressure and purified by flash chromatography (silica gel, 0-10% MeOH in CH₂Cl₂) to give tert-butyl (R)-(5-(2-amino-7-(4,5-dimethyl-4H-1,2,4-triazol-3-yl)-1H-benzo[d]imidazol-1-yl)hexyl)carbamate (26 mg, 53%).

¹H NMR (CDCl₃) δ: 7.55 (d, 1H), 7.20 (m, 1H), 6.90 (d, 1H), 4.91 (s, 1H), 4.60 (s, 3H), 3.94 (d, 1H), 3.50 (s, 3H), 3.41 (s, 3H), 3.05 (s, 3H), 2.57 (s, 3H).

### Step 6: Preparation of tert-butyl (R)-3-((1-(6-((tert-butoxycarbonyl)amino)hexan-2-yl)-7-(4,5-dimethyl-4H-1,2,4-triazol-3-yl)-1H-benzo[d]imidazol-2-yl)carbamoyl)benzoate

A solution of tert-butyl (R)-(5-(2-amino-7-(4,5-dimethyl-4H-1,2,4-triazol-3-yl)-1H-benzo[d]imidazol-1-yl)hexyl)carbamate (26 mg, 61 μmol) in DMF (1.0 mL) was added to a solution of HATU (25 mg, 67 μmol), N,N-diisopropylethylamine (21 μL, 122 μmol) and 3-(tert-butoxycarbonyl)benzoic acid (15 mg, 67 μmol) in DMF (0.25 mL) at rt. The reaction was stirred for 2 h when additional HATU (6 mg, 16 μmol), N,N-diisopropylethylamine (15 μL, 87 μmol) and 3-(tert-butoxycarbonyl)benzoic acid (5 mg, 22 μmol) were added. The reaction mixture was stirred overnight and diluted with water and purified by reverse phase chromatography (C18 column, prep-HPLC, 35-65% MeCN in water containing 0.1% TFA) to give tert-butyl (R)-3-((1-(6-((tert-butoxycarbonyl)amino)hexan-2-yl)-7-(4,5-dimethyl-4H-1,2,4-triazol-3-yl)-1H-benzo[d]imidazol-2-yl)carbamoyl)benzoate (22 mg, 57%).

LC/MS: M+H=632.3; Rt=2.92 min.

### Step 7: Preparation of (R)-3-((1-(6-aminohexan-2-yl)-7-(4,5-dimethyl-4H-1,2,4-triazol-3-yl)-1H-benzo[d]imidazol-2-yl)carbamoyl)benzoic acid

5M HCl (5.0 ml) was added to a solution of tert-butyl (R)-3-((1-(6-((tert-butoxycarbonyl)amino)hexan-2-yl)-7-(4,5-dimethyl-4H-1,2,4-triazol-3-yl)-1H-benzo[d]imidazol-2-yl)carbamoyl)benzoate (22 mg, 35 μmol) in dioxane (5.0 ml). The solution was stirred at rt for 48 h and then concentrated to dryness under reduced pressure. The product was used without further purification.

### Step 8: Preparation of (R)-1⁷-(4,5-dimethyl-4H-1,2,4-triazol-3-yl)-11-methyl-1¹H-2,6-diaza-1(2,1)-benzo[d]imidazola-4(1,3)-benzenacycloundecaphane-3,5-dione

HATU (20 mg, 52 μmol) was added to a solution of (R)-3-((1-(6-aminohexan-2-yl)-7-(4,5-dimethyl-4H-1,2,4-triazol-3-yl)-1H-benzo[d]imidazol-2-yl)carbamoyl)benzoic acid (17 mg, 35 μmol) and N,N-diisopropylethylamine (30 μl, 174 μmol) in THF (70 ml). The solution was stirred at 50° C. for 23 h and then concentrated to dryness under reduced pressure. The product was purified by reverse phase chromatography (C18 column, prep-HPLC, using 10-40% MeCN in water containing 0.1% TFA) to give (R,E)-1⁷-(4,5-dimethyl-4H-1,2,4-triazol-3-yl)-11-methyl-1²,1³-dihydro-1¹H-2,6-diaza-1(2,1)-benzo[d]imidazola-4(1,3)-benzenacycloundecaphane-3,5-dione (5 mg, 31%).

¹H NMR (CDCl₃) δ: 9.00 (s, 1H), 8.30 (d, 1H), 7.96 (d, 1H), 7.63 (m, 1H), 7.59-7.52 (m, 1H), 7.39 (m, 1H), 7.14 (d, 1H), 6.93 (s, 1H), 5.55 (s, 2H), 4.04-3.65 (m, 1H), 3.55 (s, 3H), 3.28 (s, 1H), 2.72 (s, 3H), 2.03 (s, 2H), 1.65-1.37 (m, 3H), 1.30 (s, 3H); LC/MS: M+H=458.0.

### Examples 58, 59, 60, 61: (E)-11-methyl-1⁷-(2-oxopiperidin-3-yl)-1²,1³-dihydro-1¹H-2,6-diaza-1(2,1)-benzo[d]imidazola-4(1,3)-benzenacycloundecaphane-3,5-dione

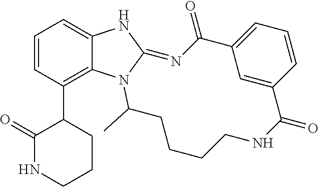 [SMILES: CC1CCCCNC(=O)c2cccc(c2)C(=O)/N=C2\Nc3cccc(C4CCCNC4=O)c3N21]

### Step 1: Preparation of 3-(2-fluoro-3-nitrophenyl)pyridin-2(1H)-one

To a solution of 3-(2-fluoro-3-nitrophenyl)-2-methoxypyridine (7.00 g, 28.2 mmol) and sodium iodide (8.45 g, 2 eq., 56.4 mmol) in MeCN (500 ml) was added chlorotrimethylsilane (7.16 mL, 2 eq., 56.4 mmol) at rt and the reaction mixture was stirred at 50° C. for 16 h. The reaction mixture was concentrated under reduced pressure. The residue was diluted with water and extracted with 10% MeOH/CH₂Cl₂. The combined organic layer dried over anhydrous Na₂SO₄, filtered and concentrated under vacuum to give 3-(2-fluoro-3-nitrophenyl)pyridin-2(1H)-one (3.20 g, 46%) as a solid.

¹H NMR (CDCl₃) δ: 8.12 (m, 1H), 7.85 (m, 1H), 7.64 (d, 1H), 7.53 (d, 1H), 7.46 (m, 1H), 6.33 (m, 1H); LC/MS: M+H=235.08.

### Step 2: Preparation tert-butyl (5-((2-nitro-6-(2-oxo-1,2-dihydropyridin-3-yl)phenyl)amino)hexyl)carbamate

To a solution of 3-(2-fluoro-3-nitrophenyl)pyridin-2(1H)-one (3.2 g, 13.7 mmol) and tert-butyl N-(5-aminohexyl)carbamate (2.96 g, 13.7 mmol) DMF (100 ml) in thionyl chloride (4.99 ml, 68.6 mmol) was added N,N-diisopropylethylamine (8.83 g, 68.3 mmol). The reaction mixture was heated at 90° C. for 16 h. The reaction mixture was poured into ice-cold water and the precipitated product was collected by filtration, washed, dried and used without further purification

¹H NMR (DMSO-d₆) δ: 8.00 (d, 1H), 7.31 (d, 1H), 7.10 (d, 1H), 6.84 (m, 1H), 6.71 (m, 1H), 6.28 (m, 1H), 3.14 (m, 1H), 2.78 (m, 2H), 1.35 (s, 9H), 0.87 (d, 3H); LC/MS: M+H=431.27.

### Step 3: Preparation of tert-butyl (5-((2-amino-6-(2-oxo-1,2-dihydropyridin-3-yl)phenyl)amino)hexyl)carbamate

To a solution of tert-butyl (5-((2-nitro-6-(2-oxo-1,2-dihydropyridin-3-yl)phenyl)amino)hexyl)carbamate (3.2 g, 13.7 mmol) in methanol was added nickel (3.41 g, 58.1 mmol). The reaction mixture was stirred under a hydrogen atmosphere for 8 h at rt. The reaction mixture was filtered through celite and the filtrate was concentrated under reduced pressure. The crude product was used without further purification.

¹H NMR (DMSO-d₆) δ: 7.46 (m, 2H), 6.78 (d, 1H), 6.65 (d, 1H), 6.37 (d, 1H), 6.31 (m, 1H), 4.09 (m, 2H), 1.37 (s, 9H), 0.070 (d, 3H); LC/MS: M+H=401.26.

### Step 4: Preparation of tert-butyl (5-(2-amino-7-(2-oxo-1,2-dihydropyridin-3-yl)-1H-benzo[d]imidazol-1-yl)hexyl)carbamate

To a solution of tert-butyl (5-((2-amino-6-(2-oxo-1,2-dihydropyridin-3-yl)phenyl)amino)hexyl)carbamate (2.50 g, 6.24 mmol) in methanol (20 ml) and MeCN (30 ml) was added cyanogen bromide (859 mg, 8.11 mmol). The reaction mixture was stirred at rt for 16 h and then concentrated to dryness under reduced pressure. The crude was dissolved in EtOAc and washed with NaHCO₃ (aq). The phases were separated, and the organic phase was dried (Na₂SO₄) and concentrated to dryness. The product (2.40 g, 90%) was used without further purification.

LC/MS: M+H=426.32.

### Step 5: Preparation of tert-butyl (5-(2-amino-7-(2-oxopiperidin-3-yl)-1H-benzo[d]imidazol-1-yl)hexyl)carbamate

To a stirred solution of tert-butyl (5-(2-amino-7-(2-oxo-1,2-dihydropyridin-3-yl)-1H-benzo[d]imidazol-1-yl)hexyl)carbamate (3.50 g, 8.22 mmol) in ethanol (20 ml) in a steel vessel was added Pd/C (10.0 g, 94.0 mmol). The reaction mixture was hydrogenated under 100 psi at 70° C. for 2 days. The reaction mixture was filtered through celite and the filtrate was concentrated under reduced pressure. The crude product was used without further purification.

LC/MS: M+H=430.51.

### Step 6: Preparation of methyl 3-((1-(6-((tert-butoxycarbonyl)amino)hexan-2-yl)-7-(2-oxopiperidin-3-yl)-1H-benzo[d]imidazol-2-yl)carbamoyl)benzoate

To a stirred solution of tert-butyl (5-(2-amino-7-(2-oxopiperidin-3-yl)-1H-benzo[d]imidazol-1-yl)hexyl)carbamate (2.80 g, 6.52 mmol) and 3-(methoxycarbonyl)benzoic acid (1.76 g, 9.78 mmol) in MeCN (150 ml) was added HATU (4.96 g, 13.0 mmol) followed by N,N-diisopropylethylamine (5.67 ml, 32.76 mmol) at 0° C. The reaction mixture was stirred at rt for 16 h. The reaction mixture was concentrated under reduced pressure and extracted with EtOAc and water. The combined organic layer was dried over anhydrous Na₂SO₄, filtered and concentrated under vacuum. The crude was purified by column chromatography (SiO₂, 100% EtOAc) to give the product (2.1 g, 34%).

LC/MS: M+H=592.3.

### Step 7: Preparation of 3-((1-(6-((tert-butoxycarbonyl)amino)hexan-2-yl)-7-(2-oxopiperidin-3-yl)-1H-benzo[d]imidazol-2-yl)carbamoyl)benzoic acid

To a stirred solution of methyl 3-((1-(6-((tert-butoxycarbonyl)amino)hexan-2-yl)-7-(2-oxopiperidin-3-yl)-1H-benzo[d]imidazol-2-yl)carbamoyl)benzoate (2.80 g, 6.52 mmol) and 3-(methoxycarbonyl)benzoic acid (1.76 g, 9.78 mmol) in THF (20 ml) and water (10 ml) was added LiOH (709 mg, 16.9 mmol). The solution was stirred at rt for 16 h. The reaction mixture was concentrated under vacuum and the crude was diluted with 10 mL of ice cold water. The aqueous solution was acidified with 50% Citric acid solution at pH:6. The white precipitate was filtered off, dried to give off white solid product (430 mg, 42%).

¹H NMR (DMSO-d₆) δ: 8.78 (s, 1H), 8.38 (d, 1H), 8.06 (d, 1H), 7.61 (m, 1H), 7.50 (d, 1H), 7.15 (m, 1H), 7.03 (m, 1H), 6.69 (m, 1H), 1.73 (d, 3H), 1.32 (s, 9H); LC/MS: M+H=578.17.

### Step 7: Preparation of 3-((1-(6-aminohexan-2-yl)-7-(2-oxopiperidin-3-yl)-1H-benzo[d]imidazol-2-yl)carbamoyl)benzoic acid

To a stirred solution of 3-((1-(6-((tert-butoxycarbonyl)amino)hexan-2-yl)-7-(2-oxopiperidin-3-yl)-1H-benzo[d]imidazol-2-yl)carbamoyl)benzoic acid (430 mg, 744 μmol) in CH₂Cl₂ (50 ml) was added 4 M HCl (9.30 ml). The solution was stirred at rt for 3 h. The reaction mixture was concentrated under reduced pressure and 370 mg crude (diastereomeric mixture) was purified by reverse phase by Prep HPLC to give 80 mg of Diastereomer 1 (91% pure) and 80 mg of Diastereomer 2 (99% pure). These two peaks were analyzed separately.

Diastereomer 1: ¹H NMR (DMSO-d₆) δ: 8.78 (s, 1H), 8.40 (d, 1H), 8.08 (d, 1H), 7.86 (s, 1H), 7.63 (m, 1H), 7.16 (m, 1H), 7.06 (m, 1H), 2.17 (m, 2H); LC/MS: M+H=478.34, rt=1.31 min.

Diastereomer 2: ¹H NMR (DMSO-d₆) δ: 8.78 (s, 1H), 8.40 (d, 1H), 8.08 (d, 1H), 7.93 (s, 1H), 7.63 (m, 1H), 7.15 (m, 1H), 7.00 (m, 1H), 2.17 (m, 2H); LC/MS: M+H=478.37, rt=1.34 min.

### Step 8: Preparation of (E)-11-methyl-1⁷-(2-oxopiperidin-3-yl)-1²,1³-dihydro-1H-2,6-diaza-1(2,1)-benzo[d]imidazola-4(1,3)-benzenacycloundecaphane-3,5-dione

To a solution of 3-((1-(6-aminohexan-2-yl)-7-(2-oxopiperidin-3-yl)-1H-benzo[d]imidazol-2-yl)carbamoyl)benzoic acid—Diastereomer 1 (80 mg, 165 μmol) in MeCN (75 ml) and DMF (25 ml) was added TCFH (70 mg, 250 μmol) and 1-methyl-1H-imidazole (97 mg, 1.17 mmol) at 0° C. The reaction mixture was stirred at rt for 2 h. The reaction mixture was quenched with 1 ml water and then concentrated under reduced pressure and the residue was triturated with water, filtered and dried. The crude was purified by preparative HPLC to afford racemic (E)-11-methyl-1⁷-(2-oxopiperidin-3-yl)-1²,1³-dihydro-1¹H-2,6-diaza-1(2,1)-benzo[d]imidazola-4(1,3)-benzenacycloundecaphane-3,5-dione. The enantiomers were separated using chiral chromatography.

### Example 58, Stereoisomer 1

¹H NMR (DMSO-d₆) @ 353.1K δ: 8.90 (bs, 1H), 8.10 (d, 1H), 7.74 (d, 1H), 7.59 (m, 2H), 7.47 (m, 2H), 7.13 (m, 1H), 7.04 (d, 1H), 4.04 (m, 1H), 3.35 (m, 2H), 2.23 (m, 1H), 1.96 (m, 4H), 1.73 (m, 3H); LC/MS: M+H=460.45.

ChiralPak IG (4.6×250) 5 um, MeOH/0.1% TFA in H2O=98:2 (v/v), Flow Rate: 1 ml/min, Rt=12.81 min

### Example 59, Stereoisomer 2

¹H NMR (DMSO-d₆) @ 353.1K δ: 8.90 (bs, 1H), 8.10 (d, 1H), 7.74 (d, 1H), 7.59 (m, 2H), 7.47 (m, 2H), 7.13 (m, 1H), 7.04 (d, 1H), 4.04 (m, 1H), 3.35 (m, 2H), 2.23 (m, 1H), 1.96 (m, 4H), 1.73 (m, 3H); LC/MS: M+H=460.45.

ChiralPak IG (4.6×250) 5 um, MeOH/0.1% TFA in H2O=98:2 (v/v), Flow Rate: 1 ml/min, Rt=17.63 min

To a solution of 3-((1-(6-aminohexan-2-yl)-7-(2-oxopiperidin-3-yl)-1H-benzo[d]imidazol-2-yl)carbamoyl)benzoic acid—Diastereomer 2 (80 mg, 165 μmol) in MeCN (75 ml) and DMF (25 ml) was added TCFH (70 mg, 250 μmol) and 1-methyl-1H-imidazole (97 mg, 1.17 mmol) at 0° C. The reaction mixture was stirred at rt for 2 h. The reaction mixture was quenched with 1 ml water and then concentrated under reduced pressure and the residue was triturated with water, filtered and dried. The crude was purified by preparative HPLC to afford racemic (E)-11-methyl-1⁷-(2-oxopiperidin-3-yl)-1²,1³-dihydro-1¹H-2,6-diaza-1(2,1)-benzo[d]imidazola-4(1,3)-benzenacycloundecaphane-3,5-dione. The enantiomers were separated using chiral chromatography.

### Example 60, Stereoisomer 3

¹H NMR (DMSO-d₆) @ 353.1K δ: 8.88 (bs, 1H), 8.10 (d, 1H), 7.72 (d, 1H), 7.55 (m, 2H), 7.43 (m, 2H), 7.09 (m, 1H), 6.97 (d, 1H), 4.07 (m, 1H), 1.68 (d, 3H); LC/MS: M+H=460.48.

ChiralPak IG (4.6×250) 5 um, Ethyl Acetate/IPA=50:50 (v/v), Flow Rate: 1 ml/min, Rt=9.36 min

### Example 61, Stereoisomer 4

¹H NMR (DMSO-d₆) @ 353.1K δ: 8.88 (bs, 1H), 8.10 (d, 1H), 7.68 (d, 1H), 7.53 (m, 2H), 7.41 (m, 2H), 7.03 (m, 1H), 6.90 (d, 1H), 4.06 (m, 1H), 1.66 (d, 3H); LC/MS: M+H=460.48.

ChiralPak IG (4.6×250) 5 um, Ethyl Acetate/IPA=50:50 (v/v), Flow Rate: 1 ml/min, Rt=11.50 min

### Examples 62 and 63: (E)-11-methyl-1⁷-(2-oxo-1,2-dihydropyridin-4-yl)-1²,1³-dihydro-1¹H-2,6-diaza-1(2,1)-benzo[d]imidazola-4(1,3)-benzenacycloundecaphane-3,5-dione

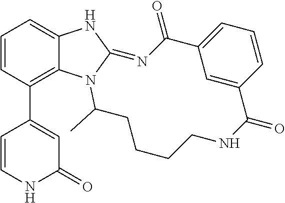 [SMILES: CC1CCCCNC(=O)c2cccc(c2)C(=O)/N=C2\Nc3cccc(-c4cc[nH]c(=O)c4)c3N21]

Prepared in the same manner as Examples 35 and 36 starting from (2-Methoxypyridin-4-yl)boronic acid and 1-Bromo-2-fluoro-3-nitrobenzene

### Example 62, Enantiomer 1

¹H NMR (DMSO-d₆) δ: 8.72 (bs, 1H), 8.10 (d, 1H), 7.72 (m, 2H), 7.58 (m, 2H), 7.49 (d, 1H), 7.23 (m, 1H), 6.99 (d, 1H), 6.39 (m, 1H), 6.30 (m, 1H), 4.48 (m, 1H), 3.61 (m, 1H); LC/MS: M+H=456.41.

ChiralPak IG (4.6×250) 5 um, IPA/Ethyl Acetate=50:50 (v/v), Flow Rate: 1 ml/min, Rt=6.47 min

### Example 63, Enantiomer 2

¹H NMR (DMSO-d₆) δ: 8.71 (bs, 1H), 8.10 (d, 1H), 7.72 (m, 2H), 7.58 (m, 2H), 7.49 (d, 1H), 7.23 (m, 1H), 7.00 (d, 1H), 6.39 (m, 1H), 6.30 (m, 1H), 4.48 (m, 1H), 3.61 (m, 1H); LC/MS: M+H=456.38.

ChiralPak IG (4.6×250) 5 um, IPA/Ethyl Acetate=50:50 (v/v), Flow Rate: 1 ml/min, Rt=10.70 min

### Example 64: (R,E)-11-methyl-N,N-bis(methyl-d₃)-3,5-dioxo-1²,1³-dihydro-1¹H-2,6-diaza-1(2,1)-benzo[d]imidazola-4(1,3)-benzenacycloundecaphane-1⁷-carboxamide

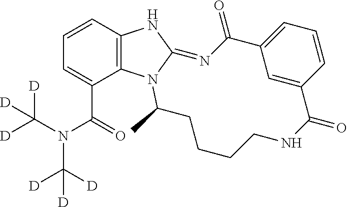 [SMILES: [2H]C([2H])([2H])N(C(=O)c1cccc2c1N1/C(=N/C(=O)c3cccc(c3)C(=O)NCCCC[C@H]1C)N2)C([2H])([2H])[2H]]

N,N-diisopropylethylamine (11 μl, 64 μmol) was added to a solution of (R)-11-methyl-3,5-dioxo-1¹H-2,6-diaza-1(2,1)-benzo[d]imidazola-4(1,3)-benzenacycloundecaphane-1⁷-carboxylic acid (17 mg, 32 μmol) and HATU (13 mg, 35 μmol) in DMF (0.75 ml) at rt. The solution was stirred for 2 min before methylamine-D₆-HCl (6 mg, 64 μmol). The reaction mixture was stirred for 1 h at rt, diluted with water (1.0 ml) and purified by reverse phase chromatography (C18 column, prep-HPLC, 14-44% MeCN in water containing 0.1% TFA) to give (R,E)-11-methyl-N,N-bis(methyl-d3)-3,5-dioxo-1²,1³-dihydro-1¹H-2,6-diaza-1(2,1)-benzo[d]imidazola-4(1,3)-benzenacycloundecaphane-1⁷-carboxamide (5 mg, 36%).

¹H NMR (DMSO-d₆) δ: 12.75 (s, 1H), 8.78 (s, 1H), 8.12 (d, 1H), 7.91-7.68 (m, 2H), 7.67-7.52 (m, 2H), 7.25 (m, 1H), 7.06 (d, 1H), 4.51 (s, 1H), 3.57 (s, 1H), 3.13-2.89 (m, 1H), 2.11-1.25 (m, 8H); LC/MS. M+H=440.0.

### Example 65: (R,E)-11-methyl-1⁷-(3-methyl-2-oxoimidazolidin-1-yl)-1²,1³-dihydro-1¹H-2,6-diaza-1(2,1)-benzo[d]imidazola-4(1,3)-benzenacycloundecaphane-3,5-dione

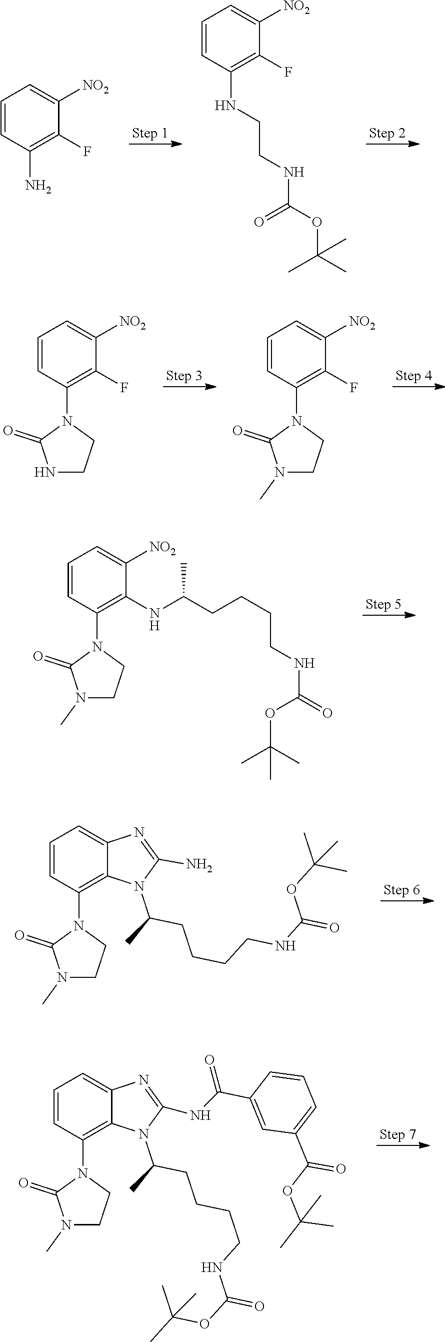

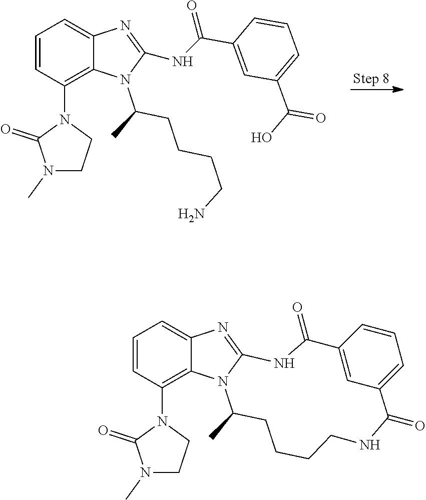 [SMILES: C[C@H](CCCCN)n1c(NC(=O)c2cccc(C(=O)O)c2)nc2cccc(N3CCN(C)C3=O)c21 ; C[C@@H]1CCCCNC(=O)c2cccc(c2)C(=O)Nc2nc3cccc(N4CCN(C)C4=O)c3n21]

### Step 1: Preparation of tert-butyl (2-((2-fluoro-3-nitrophenyl)amino)ethyl)carbamate

AcOH (800 μl) was added to a solution of 2-fluoro-3-nitroaniline (500 mg, 3.2 mmol) and tert-butyl (2-oxoethyl)carbamate (765 mg, 4.80 mmol) in MeOH (8.0 ml) at RT followed by NaCNBH3 (403 mg, 6.41 mmol). The reaction mixture was stirred for 48 h at rt and then concentrated under reduced pressure. The residue was dissolved in EtOAc (25 ml) and washed with 1 M NaOH (3×10 ml). The organic layer was dried and concentrated. Purification by reverse phase chromatography (C18 flash cartridge (Biotage 50 g), 10-60% MeCN in water with 0.1% TFA) gave tert-butyl (2-((2-fluoro-3-nitrophenyl)amino)ethyl)carbamate (464 mg, 48%).

LC/MS: M+H-tBu=244.0; Rt=2.75 min.

### Step 2: Preparation of tert-butyl (2-((2-fluoro-3-nitrophenyl)amino)ethyl)carbamate

Diphosgene (927 μl, 7.75 mmol) was slowly added to a solution of tert-butyl (2-((2-fluoro-3-nitrophenyl)amino)ethyl)carbamate (464 mg, 1.55 mmol) in CH₂Cl₂ (100 ml) at rt followed by TEA (540 μl, 3.88 mmol). The resulting solution was stirred for 5 h at rt before TFA (1.0 ml) was added. The reaction mixture was stirred at rt overnight. Additional TFA (1.0 ml) was added and stirring was continued overnight. The reaction mixture was concentrated to dryness under reduced pressure. The residue was dissolved in MeOH and then purified by flash chromatography (silica gel (Biotage 12 g), 1-10% MeOH in CH₂Cl₂ with 1% NH₃) to give 1-(2-fluoro-3-nitrophenyl)imidazolidin-2-one as a solid (176 mg, 50%).

LC/MS: M+H=226.0; Rt=1.49 min.

### Step 3: Preparation of 1-(2-fluoro-3-nitrophenyl)-3-methylimidazolidin-2-one

To a solution of 1-(2-fluoro-3-nitrophenyl)imidazolidin-2-one (88 mg, 391 μmol) in DMF (4.0 ml) was added methyl iodide (243 μl, 3.91 mmol) followed by NaH (60% in mineral oil, 78 mg, 1.95 mmol) at rt. The reaction mixture was stirred for 3 h. The reaction mixture was poured into 1M HCl (60 ml) and extracted with CH₂Cl₂ (3×20 ml). The combined organic phases were dried and concentrated. The product (61 mg) was used without further purification.

LC/MS: M+H=240.0

### Step 4: Preparation of tert-butyl (R)-(5-((2-(3-methyl-2-oxoimidazolidin-1-yl)-6-nitrophenyl)amino)hexyl)carbamate

A mixture of 1-(2-fluoro-3-nitrophenyl)-3-methylimidazolidin-2-one (94 mg, 391 μmol), tert-butyl (R)-(5-aminohexyl)carbamate (89 mg, 411 μmol) and K₂CO₃ (162 mg, 1.17 mmol) in MeCN (5.0 ml) was stirred overnight at 80° C. The reaction mixture was filtered and concentrated to dryness and purified by flash chromatography (silica gel (Biotage 15 g), 1-5% MeOH in dichloromethane with 0.1% ammonia (aq. 28%)) to give tert-butyl (R)-(5-((2-(3-methyl-2-oxoimidazolidin-1-yl)-6-nitrophenyl)amino)hexyl)carbamate (95 mg, 56%).

LC/MS: M+H=436.0; Rt=2.88 min.

### Step 5: Preparation of tert-butyl (R)-(5-(2-amino-7-(3-methyl-2-oxoimidazolidin-1-yl)-1H-benzo[d]imidazol-1-yl)hexyl)carbamate

A solution of tert-butyl (R)-(5-((2-nitro-6-(2-oxoimidazolidin-1-yl)phenyl)amino)hexyl)carbamate (95 mg, 218 μmol) in MeOH (30 ml) was hydrogenated over 10% Pd/C (12 mg, 11 μmol) at rt for 1 h. The reaction mixture was filtered and concentrated under reduced pressure. The residue was dissolved in MeOH (10 ml) and water (2.0 ml) and cyanogen bromide (25 mg, 240 μmol) was added. The resulting solution was stirred at RT for 24 h. The reaction mixture was concentrated under reduced pressure and the residue was dissolved in EtOAc and washed with 1 M NaOH (3×10 ml). The organic phase was dried and concentrated. Purification by flash chromatography (silica gel, 0-10% MeOH in CH₂Cl₂) gave tert-butyl (R)-(5-(2-amino-7-(3-methyl-2-oxoimidazolidin-1-yl)-1H-benzo[d]imidazol-1-yl)hexyl)carbamate (24 mg, 25%).

LC/MS: M+H=431.0; Rt=1.78 min.

### Step 6: Preparation of tert-butyl (R)-3-((1-(6-((tert-butoxycarbonyl)amino)hexan-2-yl)-7-(3-methyl-2-oxoimidazolidin-1-yl)-1H-benzo[d]imidazol-2-yl)carbamoyl)benzoate

A solution of HATU (25 mg, 66 μmol) and 3-(tert-butoxycarbonyl)benzoic acid (15 mg, 66 μmol) in MeCN (5.0 ml) was added to a solution of tert-butyl (R)-(5-(2-amino-7-(3-methyl-2-oxoimidazolidin-1-yl)-1H-benzo[d]imidazol-1-yl)hexyl)carbamate (24 mg, 55 μmol) and N,N-diisopropylethylamine (28 μL, 164 μmol) in MeCN (5.0 mL). The reaction was stirred overnight at rt. The reaction mixture was concentrated to dryness under reduced pressure and purified by flash chromatography (silica gel (Biotage 10 g), 10-100% EtOAc in pet. Ether) to give tert-butyl (R)-3-((1-(6-((tert-butoxycarbonyl)amino)hexan-2-yl)-7-(3-methyl-2-oxoimidazolidin-1-yl)-1H-benzo[d]imidazol-2-yl)carbamoyl)benzoate (34.4 mg, 99%).

¹H NMR (CDCl₃) δ: 8.91 (s, 1H), 8.35 (d, 1H) 8.12 (d, 1H), 7.42 (m, 1H), 7.26 (m, 2H), 7.05 (m, 1H), 5.20 (m, 1H); LC/MS: M+H=635.3.

### Step 7: Preparation of (R)-3-((1-(6-aminohexan-2-yl)-7-(3-methyl-2-oxoimidazolidin-1-yl)-1H-benzo[d]imidazol-2-yl)carbamoyl)benzoic acid

5M HCl (20 ml) was added to a solution of tert-butyl (R)-3-((1-(6-((tert-butoxycarbonyl)amino)hexan-2-yl)-7-(3-methyl-2-oxoimidazolidin-1-yl)-1H-benzo[d]imidazol-2-yl)carbamoyl)benzoate (34 mg, 54 μmol) in dioxane (20 ml). The solution was stirred overnight at rt. 5 drops of HCl (conc) was added and stirring continued overnight. The reaction mixture was concentrated to dryness under reduced pressure and used without further purification.

LC/MS: M+H=479.0; Rt=1.79 min.

### Step 8: Preparation of (R,E)-11-methyl-1⁷-(3-methyl-2-oxoimidazolidin-1-yl)-1²,1³-dihydro-1¹H-2,6-diaza-1(2,1)-benzo[d]imidazola-4(1,3)-benzenacycloundecaphane-3,5-dione

HATU (31 mg, 81 μmol) and N,N-dissopropylethylamine (47 μl, 271 μmol) were added to a solution of (R)-3-((1-(6-aminohexan-2-yl)-7-(3-methyl-2-oxoimidazolidin-1-yl)-1H-benzo[d]imidazol-2-yl)carbamoyl)benzoic acid (28 mg, 54 μmol) in THF (108 ml). The reaction mixture was stirred at rt for 4 days and then concentrated to dryness. The residue was dissolved in DMSO (3.0 ml) and purified by reverse phase chromatography (C18 column, 20-50% MeCN in water containing 0.1% formic acid) to give (R)-11-methyl-1⁷-(3-methyl-2-oxoimidazolidin-1-yl)-1H-2,6-diaza-1(2,1)-benzo[d]imidazola-4(1,3)-benzenacycloundecaphane-3,5-dione (3.8 mg, 15%).

¹H NMR (methanol-d₄) δ: 8.94 (s, 1H), 8.26-8.15 (m, 1H), 7.82 (d, 1H), 7.60 (tm, 1H), 7.47 (d, 1H), 7.28 (m, 1H), 7.18-7.12 (m, 1H), 5.57-5.03 (m, 1H), 4.07-3.52 (m, 5H), 3.22-3.05 (m, 1H), 2.92 (d, 3H), 2.30-1.84 (m, 3H), 1.77 (s, 3H), 1.70-1.40 (m, 3H); LC/MS: M+H=461.0.

### Example 66: (R,E)-11-methyl-1⁷-(1-methyl-1H-1,2,3-triazol-4-yl)-1²,1³-dihydro-1¹H-2,6-diaza-1(2,1)-benzo[d]imidazola-4(1,3)-benzenacycloundecaphane-3,5-dione

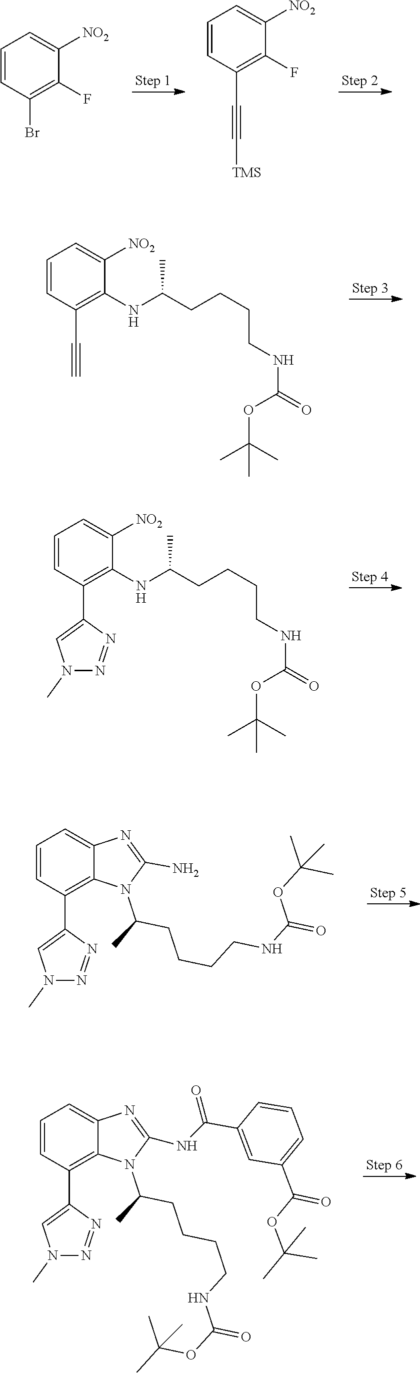

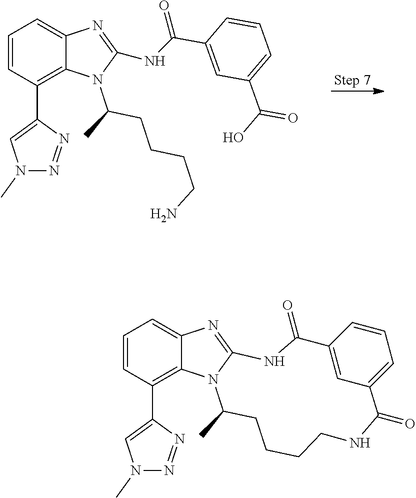 [SMILES: C[C@H](CCCCN)n1c(NC(=O)c2cccc(C(=O)O)c2)nc2cccc(-c3cn(C)nn3)c21 ; C[C@@H]1CCCCNC(=O)c2cccc(c2)C(=O)Nc2nc3cccc(-c4cn(C)nn4)c3n21]

### Step 1: Preparation of ((2-fluoro-3-nitrophenyl)ethynyl)trimethylsilane

TMS-acetylene (194 μl, 1.36 mmol) followed by N,N-diisopropylethylamine (470 μl, 2.73 mmol) were added to a degassed mixture of 1-bromo-2-fluoro-3-nitrobenzene (200 mg, 909 μmol), Bis(triphenylphosphine)palladium(II) dichloride (32 mg, 45 μmol) and CuI (9 mg, 46 μmol) in THF (7.0 ml) under argon. The reaction mixture was stirred at rt for 3 days and then concentrated to dryness. The residue was dissolved in EtOAc and washed with 1M HCl and brine. The organic phase was dried, concentrated under reduced pressure and purified by flash chromatography (silica gel, 0-10% EtOAc/Pet-ether) to give ((2-fluoro-3-nitrophenyl)ethynyl)trimethylsilane (140 mg, 65%).

### Step 2: Preparation of tert-butyl (R)-(5-((2-ethynyl-6-nitrophenyl)amino)hexyl)carbamate

A solution of ((2-fluoro-3-nitrophenyl)ethynyl)trimethylsilane (107 mg, 451 μmol) and tert-butyl (R)-(5-aminohexyl)carbamate (132 mg, 609 μmol) in MeCN (5.0 ml) was stirred overnight at rt. The reaction mixture was concentrated to dryness and used without further purification.

### Step 3: Preparation of tert-butyl (R)-(5-((2-(1-methyl-1H-1,2,3-triazol-4-yl)-6-nitrophenyl)amino)hexyl)carbamate

To a flask charged with sodium azide (49 mg, 753 μmol), copper sulfate pentahydrate (22 mg, 89 μmol), ascorbic acid (31 mg, 177 μmol) and K₂CO₃ (306 mg, 2.21 mmol) in t-BuOH (2.50 ml) and water (2.50 ml) was added a solution of tert-butyl (R)-(5-((2-ethynyl-6-nitrophenyl)amino)hexyl)carbamate (160 mg, 443 μmol) in dioxane. The reaction mixture was stirred for 1 h and then Mel (41 μl, 664 μmol) was added. The reaction mixture was stirred for 72 h and then diluted with EtOAc. The organic phase was washed with brine, dried and concentrated. The residue was purified by flash chromatography (silica gel, 25-75% EtOAc/Pet-ether) to give tert-butyl (R)-(5-((2-(1-methyl-1H-1,2,3-triazol-4-yl)-6-nitrophenyl)amino)hexyl)carbamate (87 mg, 47%).

¹H NMR (CDCl₃) δ: 7.98 (m, 1H), 7.90 (s, 1H), 7.75 (m, 1H), 6.99 (d, 1H), 6.92 (m, 1H), 4.52 (s, 1H), 4.20 (s, 3H), 3.11-2.93 (m, 3H), 1.43 (s, 10H), 1.38-1.14 (m, 7H), 0.96 (d, 3H).

### Step 4: Preparation of tert-butyl (R)-(5-(2-amino-7-(1-methyl-1H-1,2,3-triazol-4-yl)-1H-benzo[d]imidazol-1-yl)hexyl)carbamate

10% Pd/C (11 mg, 10 μmol) was added to a solution of tert-butyl (R)-(5-((2-(1-methyl-1H-1,2,3-triazol-4-yl)-6-nitrophenyl)amino)hexyl)carbamate (87 mg, 208 μmol) in methanol (5.0 ml). The reaction flask was evacuated and flushed with hydrogen twice. The mixture was stirred under hydrogen for 1.5 hours. The mixture was filtered and MeOH (5.0 ml), water (1.0 ml) and cyanogen bromide (26 mg, 249 μmol) were added to the solution and the mixture was stirred for 48 h at RT. The reaction mixture was concentrated under reduced pressure and the residue was purified by flash chromatography (silica gel, CH₂Cl₂/1-5% MeOH in CH₂Cl₂ with 0.1% ammonia (aq. 28%) to give tert-butyl (R)-(5-(2-amino-7-(1-methyl-1H-1,2,3-triazol-4-yl)-1H-benzo[d]imidazol-1-yl)hexyl)carbamate (42 mg, 49%).

¹H NMR (CDCl₃) δ: 7.67 (s, 1H), 7.45 (m, 1H), 7.11 (m, 1H), 6.88 (m, 1H), 4.88-4.68 (m, 3H), 4.44 (m, 1H), 4.21 (s, 3H), 3.01 (m, 2H), 2.11-1.80 (m, 2H), 1.68-1.53 (m, 1H), 1.41 (s, 1¹H), 1.30 (m, 2H), 1.16-0.90 (m, 2H).

### Step 5: Preparation of tert-butyl (R)-3-((1-(6-((tert-butoxycarbonyl)amino)hexan-2-yl)-7-(1-methyl-1H-1,2,3-triazol-4-yl)-1H-benzo[d]imidazol-2-yl)carbamoyl)benzoate

A solution of tert-butyl (R)-(5-(2-amino-7-(1-methyl-1H-1,2,3-triazol-4-yl)-1H-benzo[d]imidazol-1-yl)hexyl)carbamate (42 mg, 102 μmol) in MeCN (2.5 ml) was added to a solution of HATU (43 mg, 112 μmol), 3-(tert-butoxycarbonyl)benzoic acid (25 mg, 112 μmol) and N,N-diisopropylethylamine (35 μL, 203 μmol) in MeCN (0.5 ml). The reaction was stirred overnight at RT. The reaction mixture was concentrated to dryness under reduced pressure and purified by flash chromatography (silica gel (Biotage 10 g), 0-5% MeOH in CH₂Cl₂) to give tert-butyl (R)-3-((1-(6-((tert-butoxycarbonyl)amino)hexan-2-yl)-7-(1-methyl-1H-1,2,3-triazol-4-yl)-1H-benzo[d]imidazol-2-yl)carbamoyl)benzoate (54 mg, 86%).

¹H NMR (CDCl₃) δ: 12.85 (s, 1H), 8.92 (s, 1H), 8.44 (d, 1H), 8.13 (d, 1H), 7.75 (s, 1H), 7.51 (m, 1H), 7.37 (d, 1H), 7.25 (d, 1H), 7.10 (d, 1H), 4.64 (s, 1H), 4.27 (s, 4H), 2.98 (d, 2H), 2.64 (s, 1H), 1.78-1.60 (m, 13H), 1.39 (s, 12H).

### Step 6: Preparation of (R)-3-((1-(6-aminohexan-2-yl)-7-(1-methyl-1H-1,2,3-triazol-4-yl)-1H-benzo[d]imidazol-2-yl)carbamoyl)benzoic acid

5 M HCl (5.0 ml) was added to a solution of tert-butyl (R)-3-((1-(6-((tert-butoxycarbonyl)amino)hexan-2-yl)-7-(1-methyl-1H-1,2,3-triazol-4-yl)-1H-benzo[d]imidazol-2-yl)carbamoyl)benzoate (54 mg, 87 μmol) in dioxane (5.0 ml). The reaction mixture was stirred for 3 days at rt. The volatiles were removed under reduced pressure and the product was used without further purification.

### Step 7: Preparation of (R)-11-methyl-1⁷-(1-methyl-1H-1,2,3-triazol-4-yl)-1¹H-2,6-diaza-1(2,1)-benzo[d]imidazola-4(1,3)-benzenacycloundecaphane-3,5-dione

N,N-diisopropylethylamine (45 μl, 262 μmol) and HATU (50 mg, 131 μmol) were added to a suspension of (R)-3-((1-(6-aminohexan-2-yl)-7-(1-methyl-1H-1,2,3-triazol-4-yl)-1H-benzo[d]imidazol-2-yl)carbamoyl)benzoic acid (40 mg, 87 μmol) in THF (180 ml). The reaction mixture was stirred for 72 h at rt when N,N-diisopropylethylamine (45 μl, 262 μmol) and HATU (50 mg, 131 μmol) were added. The reaction mixture was stirred for an additional 6 h at rt and then purified using preparative HPLC (10-30% MeCN/H₂O with 0.1% TFA) to give (R)-11-methyl-1⁷-(1-methyl-1H-1,2,3-triazol-4-yl)-1¹H-2,6-diaza-1(2,1)-benzo[d]imidazola-4(1,3)-benzenacycloundecaphane-3,5-dione (2 mg, 5%).

¹H NMR (CDCl₃) δ: 9.00 (s, 1H), 8.30 (d, 1H), 7.95 (d, 1H), 7.75 (s, 1H), 7.62 (m, 1H), 7.42 (d, 1H), 7.28-7.22 (m, 1H), 7.13 (d, 1H), 7.01 (s, 1H), 4.63 (s, 1H), 4.28 (s, 3H), 4.05-3.73 (m, 1H), 3.21 (s, 2H), 2.15-1.99 (m, 3H), 2.00-1.85 (m, 2H), 1.61 (s, 4H), 1.48 (s, 1H); LC/MS: M+H=440.0.

### Examples 67 and 68: (E)-11-ethyl-N,N-dimethyl-3,5-dioxo-1²,1³-dihydro-1¹H-2,6-diaza-1(2,1)-benzo[d]imidazola-4(1,3)-benzenacycloundecaphane-1⁷-carboxamide

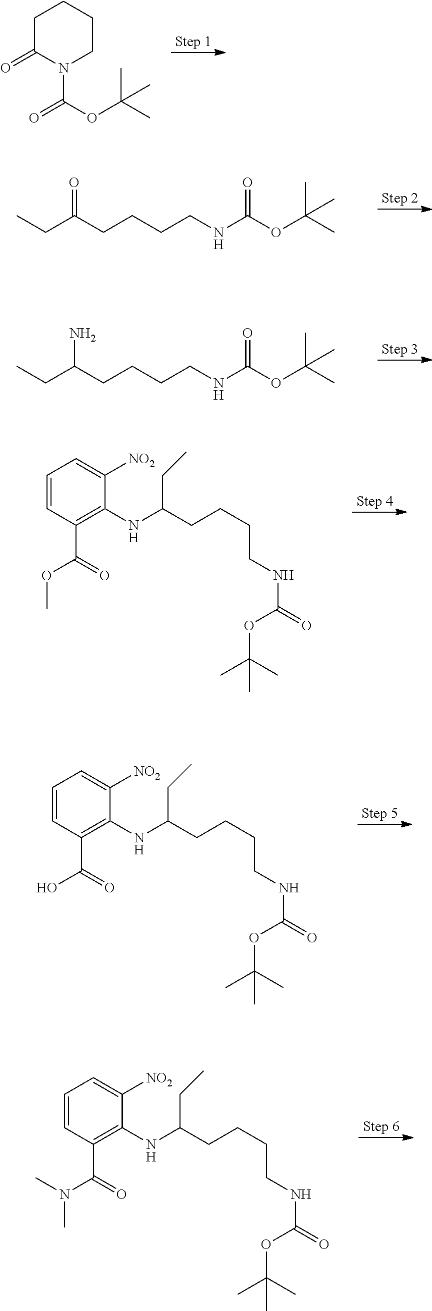

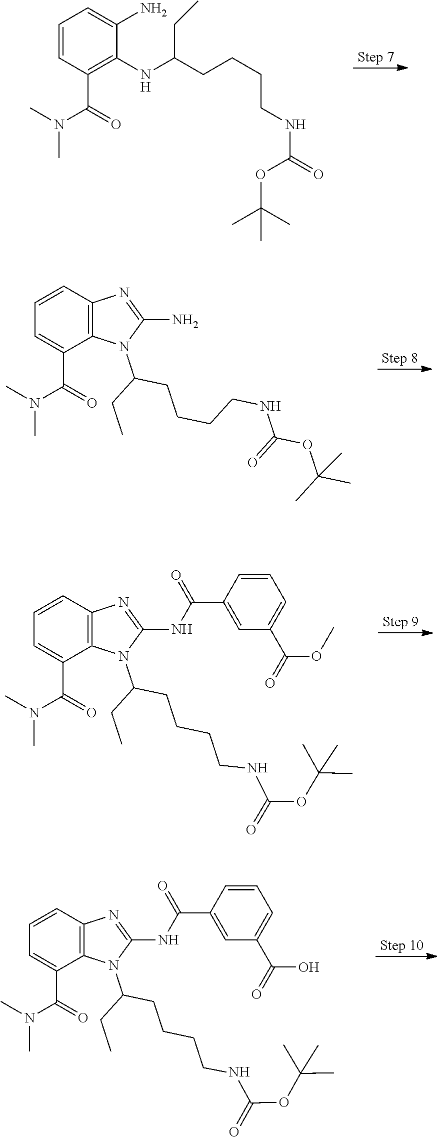

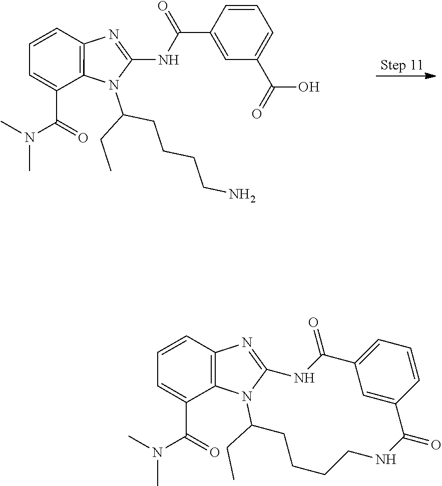 [SMILES: CCC(CCCCN)n1c(NC(=O)c2cccc(C(=O)O)c2)nc2cccc(C(=O)N(C)C)c21 ; CCC1CCCCNC(=O)c2cccc(c2)C(=O)Nc2nc3cccc(C(=O)N(C)C)c3n21]

### Step 1: Preparation tert-butyl tert-butyl (5-oxoheptyl)carbamate

To a solution of tert-butyl 2-oxopiperidine-1-carboxylate (20.0 g, 100 mmol) in THF (200 ml) at −78° C. was slowly added 3 M bromo(ethyl)magnesium (36.8 ml, 110 mmol). The solution was stirred at −78° C. for 3 h and quenched with 1M HCl. The reaction mixture was extracted with EtOAc. The organic phase was dried (Na₂SO₄) and concentrated. The product was used without further purification.

¹H NMR (DMSO-d₆) δ: 6.78 (m, 1H), 2.87 (m, 2H), 2.39 (m, 5H), 0.90 (m, 3H).

### Step 2: Preparation of tert-butyl (5-aminoheptyl)carbamate

A solution of tert-butyl (5-oxoheptyl)carbamate (20.0 g, 87.2 mmol) and Pd/C (12.0 g, 113 mmol) in methanolic ammonia (500 ml) was stirred under a hydrogen atmosphere (60 psi) at rt for 16 h in a steel apparatus. The reaction mixture was filtered through a pad of celite, washed with methanol and concentrated under vacuum. The crude residue was purified by combiflash chromatography (SiO₂, 0 to 10% Methanolic ammonia in CH₂Cl₂) to give tert-butyl (5-aminoheptyl)carbamate.

¹H NMR (DMSO-d₆) δ: 6.76 (m, 1H), 3.16 (s, 1H), 2.88 (m, 2H), 1.15 (m, 2H), 0.83 (m, 3H).

### Step 3: Preparation of methyl 2-((7-((tert-butoxycarbonyl)amino)heptan-3-yl)amino)-3-nitrobenzoate

To a stirred solution of methyl 2-fluoro-3-nitrobenzoate (10.0 g, 50.2 mmol) in DMF (100 ml) was added tert-butyl (5-aminoheptyl)carbamate (11.6 g, 50.2 mmol) and N,N-diisopropylethylamine (19.5 g, 151 mmol) at rt. The reaction mixture was stirred at 80° C. for 16 h. The reaction mixture was poured into ice cold water and extracted with EtOAc. The combined organic layer was dried over anhydrous Na₂SO₄, filtered and concentrated under vacuum. The crude product was used without further purification.

¹H NMR (DMSO-d₆) δ: 8.08 (m, 1H), 6.83 (m, 1H), 3.86 (s, 3H), 2.85 (m, 2H), 1.36 (s, 9H), 0.76 (m, 3H); LC/MS: M+H=410.39.

### Step 4: Preparation of 2-((7-((tert-butoxycarbonyl)amino)heptan-3-yl)amino)-3-nitrobenzoic acid

To a stirred solution of methyl 2-((7-((tert-butoxycarbonyl)amino)heptan-3-yl)amino)-3-nitrobenzoate (20.0 g, 48.8 mmol) in THF (100 ml) and water (100 ml) was added LiOH (10.2 g, 244 mmol). The solution was stirred at rt for 16 h. The reaction mixture was acidified with 1N HCl and extracted with EtOAc. The combined organic layers was dried over anhydrous Na₂SO₄, filtered and concentrated under reduced pressure. The product was used without further purification.

¹H NMR (DMSO-d₆) δ: 8.45 (m, 1H), 8.07 (m, 1H), 7.90 (m, 1H), 6.78 (m, 1H), 2.84 (m, 2H), 1.36 (s, 9H), 0.75 (m, 3H); LC/MS: M−H=394.20.

### Step 5: Preparation of tert-butyl (5-((2-(dimethylcarbamoyl)-6-nitrophenyl)amino)heptyl)carbamate

To a stirred solution of 2-((7-((tert-butoxycarbonyl)amino)heptan-3-yl)amino)-3-nitrobenzoic acid (10.0 g, 25.3 mmol) in DMF (100 ml) was added dimethylamine (3.42 g, 75.9 mmol), HATU (19.2 g, 244 mmol) and N,N-diisopropylethylamine (16.3 g, 25.3 mmol) at 0° C. The reaction mixture was stirred at rt for 6 h. The reaction mixture was poured into ice cold water and extracted with EtOAc. The combined organic layers were dried over anhydrous Na₂SO₄, filtered and concentrated under reduced pressure. The product was used without further purification.

¹H NMR (DMSO-d₆) δ: 8.11 (d, 1H) 7.95 (s, 1H), 7.66 (d, 1H), 7.37 (d, 1H), 6.79 (m, 3H), 4.02 (m, 1H), 3.62 (m, 1H), 3.15 (m, 1H), 1.31 (s, 9H); LC/MS: M+H=423.18.

### Step 6: Preparation of tert-butyl (5-((2-amino-6-(dimethylcarbamoyl)phenyl)amino)heptyl)carbamate

To a stirred solution of tert-butyl (5-((2-(dimethylcarbamoyl)-6-nitrophenyl)amino)heptyl)carbamate (300 mg, 710 μmol) in MeOH (6.0 ml) was added Pd/C (150 mg, 1.41 mmol). The reaction mixture stirred under a hydrogen atmosphere for 3 h at rt. The reaction mixture was filtered through celite bed and concentrated under reduced pressure. The product was used without further purification.

¹H NMR (DMSO-d₆) δ: 6.75 (s, 1H), 6.65 (s, 2H), 6.37 (s, 1H), 4.02 (m, 1H), 3.03 (s, 3H), 2.86 (s, 3H), 1.36 (s, 9H), 0.78 (m, 3H); LC/MS: M+H=423.18.

### Step 7: Preparation of tert-butyl (5-(2-amino-7-(dimethylcarbamoyl)-1H-benzo[d]imidazol-1-yl)heptyl)carbamate

To a stirred solution of tert-butyl (5-((2-amino-6-(dimethylcarbamoyl)phenyl)amino)heptyl)carbamate (7.5 g, 19.1 mmol) in MeOH (15 ml), MeCN (15 ml) and water (15 ml) was added cyanogen bromide (2.23 g, 21.0 mmol) at rt. The reaction mixture was stirred for 5 h at rt. The crude reaction mixture was concentrated under reduced pressure and basified with saturated sodium bicarbonate solution and extracted with EtOAc. The combined organic layers were dried over anhydrous Na₂SO₄, filtered and concentrated under reduced pressure. The crude product (5.50 g) was used without further purification.

¹H NMR (DMSO-d₆) δ: 7.16 (s, 1H), 6.98 (m, 1H), 6.75 (m, 1H), 6.68 (d, 1H), 6.41 (s, 1H), 4.04 (m, 1H), 3.04 (s, 3H), 2.81 (s, 3H), 1.79 (m, 2H), 1.33 (s, 9H), 0.69 (m, 3H); LC/MS: M+H=418.27.

### Step 8: Preparation of methyl 3-((1-(7-((tert-butoxycarbonyl)amino)heptan-3-yl)-7-(dimethylcarbamoyl)-1H-benzo[d]imidazol-2-yl)carbamoyl)benzoate

To a stirred solution of tert-butyl (5-(2-amino-7-(dimethylcarbamoyl)-1H-benzo[d]imidazol-1-yl)heptyl)carbamate (5.5 g, 13.2 mmol) and 3-(methoxycarbonyl)benzoic acid (2.85 g, 15.8 mmol) in MeCN (60 ml) was added TCFH (5.54 g, 19.80 mmol) and 1-methyl-1H-imidazole (5.41 g, 65.9 mmol) at 0° C. The reaction mixture was stirred at rt for 6 h. The crude reaction mixture was added to ice cold water and extracted with EtOAC. The combined organic layers were dried over anhydrous Na₂SO₄, filtered and concentrated under vacuum. The crude product (6.0 g) was used without further purification.

¹H NMR (DMSO-d₆) δ: 8.79 (s, 1H), 8.39 (d, 1H), 8.09 (d, 1H), 7.64 (m, 2H), 7.25 (m, 1H), 7.07 (m, 1H), 4.15 (m, 1H), 3.89 (s, 3H), 3.10 (s, 3H), 2.85 (s, 3H), 1.94 (m, 2H), 1.28 (s, 9H), 0.75 (m, 3H); LC/MS: M+H=580.36.

### Step 9: Preparation of 3-((1-(7-((tert-butoxycarbonyl)amino)heptan-3-yl)-7-(dimethylcarbamoyl)-1H-benzo[d]imidazol-2-yl)carbamoyl)benzoic acid

To a stirred solution of methyl 3-((1-(7-((tert-butoxycarbonyl)amino)heptan-3-yl)-7-(dimethylcarbamoyl)-1H-benzo[d]imidazol-2-yl)carbamoyl)benzoate (6.0 g, 10.4 mmol in THF (30 ml) and water (30 ml) was added KOH (1.74 g, 31.1 mmol). The solution was stirred at rt for 5 h. The reaction mixture was concentrated under reduced pressure and extracted with EtOAc. The combined organic layers were dried over anhydrous Na₂SO₄, filtered and concentrated under reduced pressure. The product (5.0 g) was used without further purification.

¹H NMR (DMSO-d₆) δ: 8.76 (s, 1H), 8.38 (d, 1H), 8.08 (d, 1H), 7.62 (m, 2H), 7.25 (m, 1H), 7.06 (m, 1H), 4.17 (m, 1H), 3.10 (s, 3H), 2.85 (s, 3H), 1.98 (m, 2H), 1.28 (s, 9H), 0.76 (m, 3H); LC/MS: M−H=564.30.

### Step 9: Preparation of 3-((1-(7-aminoheptan-3-yl)-7-(dimethylcarbamoyl)-1H-benzo[d]imidazol-2-yl)carbamoyl)benzoic acid

To a stirred solution of 3-((1-(7-((tert-butoxycarbonyl)amino)heptan-3-yl)-7-(dimethylcarbamoyl)-1H-benzo[d]imidazol-2-yl)carbamoyl)benzoic acid (3.6 g, 6.36 mmol) in CH₂Cl₂ (40 ml) was added HCl in dioxane (5.0 ml) at 0° C. The solution was stirred at rt for 5 h. The reaction mixture was concentrated under reduced pressure and the residue was washed with diethyl ether. The product (3.0 g) was used without further purification.

¹H NMR (DMSO-d₆) δ: 8.76 (s, 1H), 8.38 (d, 1H), 8.08 (d, 1H), 7.91 (bs, 1H), 7.81 (bs, 1H), 7.63 (m, 2H), 7.26 (m, 1H), 7.09 (m, 1H), 4.17 (m, 1H), 3.11 (s, 3H), 2.86 (s, 3H), 1.95 (m, 2H), 0.76 (m, 3H); LC/MS: M+H=466.26

### Step 10: Preparation of (E)-11-ethyl-N,N-dimethyl-3,5-dioxo-1²,1³-dihydro-1¹H-2,6-diaza-1(2,1)-benzo[d]imidazola-4(1,3)-benzenacycloundecaphane-1⁷-carboxamide

To a stirred solution of 3-((1-(7-aminoheptan-3-yl)-7-(dimethylcarbamoyl)-1H-benzo[d]imidazol-2-yl)carbamoyl)benzoic acid (150 mg, 322 μmol) in DMF (1.0 ml) and MeCN (150 ml) was added TCFH (181 mg, 644 μmol) and 1-methyl-1H-imidazole (132 mg, 1.61 mmol) dropwise at 0° C. The reaction mixture was stirred at rt for 2 h. The reaction mixture was concentrated to dryness under reduced pressure and the residue using reverse phase Prep HPLC to afford racemic (E)-11-ethyl-N,N-dimethyl-3,5-dioxo-1²,1³-dihydro-1¹H-2,6-diaza-1(2,1)-benzo[d]imidazola-4(1,3)-benzenacycloundecaphane-1⁷-carboxamide (50 mg). The racemic product was submitted to chiral purification.

### Example 67, Enantiomer 1

¹H NMR (DMSO-d₆) (T=353 K) δ: 8.85 (bs, 1H), 8.10 (d, 1H), 7.75 (d, 1H), 7.57 (m, 2H), 7.51 (bs, 1H), 7.23 (m, 1H), 7.04 (d, 1H), 2.87 (s, 3H), 1.85 (m, 4H); LC/MS: M+H=448.47.

Lux Amylose-1 (4.6×250) 5 um, 0.1% TEA in n-Hexane/EtOH=20:80 (v/v), flow rate: 1 ml/min, Rt=7.38 min.

### Example 68, Enantiomer 2

¹H NMR (DMSO-d₆) (T=353 K) δ: 8.85 (bs, 1H), 8.10 (d, 1H), 7.75 (d, 1H), 7.57 (m, 2H), 7.51 (bs, 1H), 7.22 (m, 1H), 7.03 (d, 1H), 2.87 (s, 3H), 1.85 (m, 4H); LC/MS: M+H=448.47.

Lux Amylose-1 (4.6×250) 5 um, 0.1% TEA in n-Hexane/EtOH=20:80 (v/v), flow rate: 1 ml/min, Rt=9.29 min.

### Example 69: (R,E)-1⁵-(2-hydroxypropan-2-yl)-N,N,11-trimethyl-3,5-dioxo-1²,1³-dihydro-1¹H-2,6-diaza-1(2,1)-benzo[d]imidazola-4(1,3)-benzenacycloundecaphane-1⁷-carboxamide

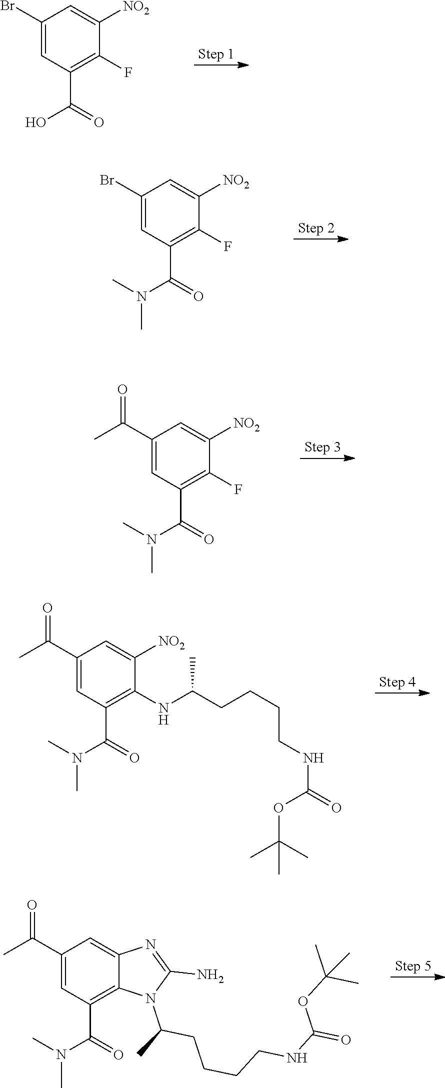 [SMILES: O=C(O)c1cc(Br)cc([N+](=O)[O-])c1F ; CN(C)C(=O)c1cc(Br)cc([N+](=O)[O-])c1F ; CC(=O)c1cc(C(=O)N(C)C)c(F)c([N+](=O)[O-])c1 ; CC(=O)c1cc(C(=O)N(C)C)c(N[C@H](C)CCCCNC(=O)OC(C)(C)C)c([N+](=O)[O-])c1 ; CC(=O)c1cc(C(=O)N(C)C)c2c(c1)nc(N)n2[C@H](C)CCCCNC(=O)OC(C)(C)C]

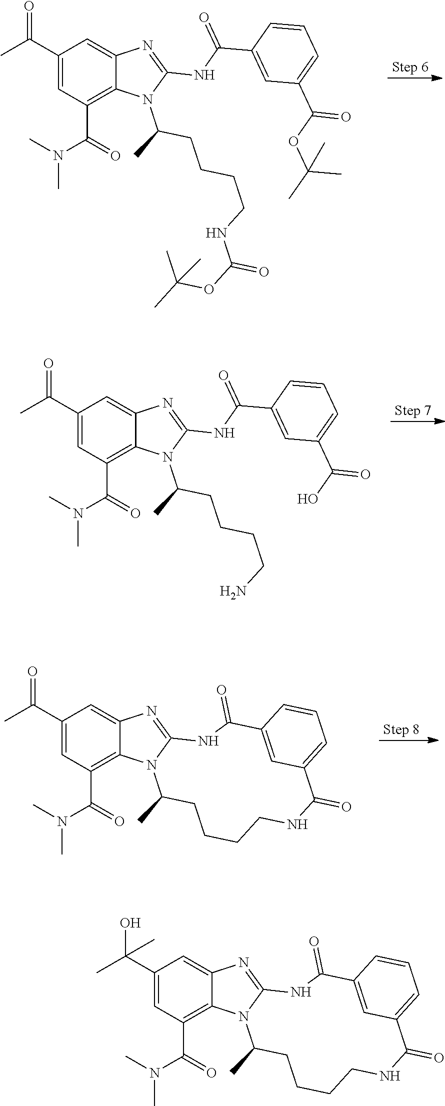 [SMILES: CC(=O)c1cc(C(=O)N(C)C)c2c(c1)nc(NC(=O)c1cccc(C(=O)OC(C)(C)C)c1)n2[C@H](C)CCCCNC(=O)OC(C)(C)C ; CC(=O)c1cc(C(=O)N(C)C)c2c(c1)nc(NC(=O)c1cccc(C(=O)O)c1)n2[C@H](C)CCCCN ; CC(=O)c1cc(C(=O)N(C)C)c2c(c1)nc1n2[C@H](C)CCCCNC(=O)c2cccc(c2)C(=O)N1 ; C[C@@H]1CCCCNC(=O)c2cccc(c2)C(=O)Nc2nc3cc(C(C)(C)O)cc(C(=O)N(C)C)c3n21]

### Step 1: Preparation of 5-bromo-2-fluoro-N,N-dimethyl-3-nitrobenzamide

Oxalyl chloride (180 μl, 1.91 mmol) followed by DMF (20 μl) were added to a suspension of 5-bromo-2-fluoro-3-nitrobenzoic acid (503 mg, 1.91 mmol) in CH₂Cl₂ (20 ml). The reaction mixture was stirred at rt for 2 h and then the volatiles were removed under reduced pressure. The residue was evaporated once from toluene. The residue was dissolved in CH₂Cl₂ (15 ml) and 2 M methylamine in THF (953 μl, 1.91 mmol) and N,N-diisopropylethylamine (394 μl, 2.29 mmol) were added. The reaction mixture was stirred overnight at RT, concentrated and purified by flash chromatography (silica gel (Biotage 10 g), 10-50% EtOAc/pet. ether) to give 5-bromo-2-fluoro-N,N-dimethyl-3-nitrobenzamide 434 mg, 78%).

¹H NMR (CDCl₃) δ: 8.23 (m, 1H), 7.81 (m, 1H), 3.16 (s, 3H), 2.99 (d, 3H).

### Step 2: Preparation of 5-acetyl-2-fluoro-N,N-dimethyl-3-nitrobenzamide

Pd(PPh₃)₂Cl₂ (31 mg, 45 μmol) was added to a degassed solution of 5-bromo-2-fluoro-N,N-dimethyl-3-nitrobenzamide (236 mg, 811 μmol) and tributyl(1-ethoxyvinyl)stannane (351 mg, 973 μmol) in dioxane (8.0 ml) in a vial. The sealed vial was heated overnight at 100° C. The reaction mixture was cooled to RT and 1M HCl (1.5 ml) was added. The reaction mixture was stirred at rt for 1.5 h and the volatiles were removed under reduced pressure. The residue was dissolved in CH₂Cl₂ and washed with 1M NaF(aq). Solids were removed by filtration and the organic phase was dried and concentrated. Purification by flash chromatography (silica gel (Biotage 10 g), 40-100% EtOAc/pet. ether) gave 5-acetyl-2-fluoro-N,N-dimethyl-3-nitrobenzamide (178 mg, 86%).

¹H NMR (CDCl₃) δ: 8.66 (m, 1H), 8.27 (m, 1H), 3.19 (s, 3H), 2.99 (d, 3H), 2.67 (s, 3H).

### Step 3: Preparation of tert-butyl (R)-(5-((4-acetyl-2-(dimethylcarbamoyl)-6-nitrophenyl)amino)hexyl)carbamate

A mixture of 5-acetyl-2-fluoro-N,N-dimethyl-3-nitrobenzamide, K₂CO₃ (56 mg, 409 μmol) and tert-butyl (R)-(5-aminohexyl)carbamate (44 mg, 205 μmol) in MeCN (10 ml) was stirred overnight at rt. The reaction mixture was filtered and concentrated to dryness under reduced pressure. The product, tert-butyl (R)-(5-((4-acetyl-2-(dimethylcarbamoyl)-6-nitrophenyl)amino)hexyl)carbamate, was used without further purification.

### Step 4: Preparation of tert-butyl (R)-(5-(5-acetyl-2-amino-7-(dimethylcarbamoyl)-1H-benzo[d]imidazol-1-yl)hexyl)carbamate

10% Pd/C (11 mg, 10 μmol) was added to a solution of tert-butyl (R)-(5-((4-acetyl-2-(dimethylcarbamoyl)-6-nitrophenyl)amino)hexyl)carbamate (92 mg, 204 μmol) in methanol (15.0 ml). The reaction flask was evacuated and flushed with hydrogen twice. The mixture was stirred under hydrogen for 4 hours. The mixture was filtered into a solution of cyanogen bromide (26 mg, 245 μmol) in water (2.0 ml). The reaction mixture was stirred for 20 h and cyanogen bromide (5 mg, 49 μmol) was added and the mixture was stirred for 6 h at 40° C. The reaction mixture was concentrated under reduced pressure and the residue was purified by flash chromatography (silica gel, CH₂Cl₂/0-5% MeOH in CH₂Cl₂ with 0.1% ammonia (aq. 28%) to give tert-butyl (R)-(5-(5-acetyl-2-amino-7-(dimethylcarbamoyl)-1H-benzo[d]imidazol-1-yl)hexyl)carbamate (46 mg, 50%).

¹H NMR (CDCl₃) δ: 8.66 (m, 1H), 8.27 (m, 1H), 3.19 (s, 3H), 2.99 (d, 3H), 2.67 (s, 3H).

### Step 5: Preparation of tert-butyl (R)-3-((5-acetyl-1-(6-((tert-butoxycarbonyl)amino)hexan-2-yl)-7-(dimethylcarbamoyl)-1H-benzo[d]imidazol-2-yl)carbamoyl)benzoate

A solution of tert-butyl (R)-(5-(5-acetyl-2-amino-7-(dimethylcarbamoyl)-1H-benzo[d]imidazol-1-yl)hexyl)carbamate (46 mg, 102 μmol) in MeCN (2.5 ml) was added to a solution of HATU (43 mg, 112 μmol), 3-(tert-butoxycarbonyl)benzoic acid (25 mg, 112 μmol) and N,N-diisopropylethylamine (35 μL, 203 μmol) in MeCN (0.5 ml). The reaction was stirred overnight at rt. The reaction mixture was concentrated to dryness under reduced pressure and purified by flash chromatography (silica gel (Biotage 10 g), 0-5% MeOH in CH₂Cl₂) to give tert-butyl (R)-3-((5-acetyl-1-(6-((tert-butoxycarbonyl)amino)hexan-2-yl)-7-(dimethylcarbamoyl)-1H-benzo[d]imidazol-2-yl)carbamoyl)benzoate (51 mg, 77%).

¹H NMR (CDCl₃) δ: 8.89 (s, 1H), 8.40 (m, 1H), 8.16 (m, 1H), 7.96 (s, 1H), 7.75 (s, 1H), 7.53 (m, 1H), 4.74-4.23 (m, 1H), 3.25 (s, 3H), 3.11-2.82 (m, 5H), 2.61 (d, 4H), 2.05 (s, 1H), 1.79 (d, 3H), 1.62 (d, 9H), 1.38 (s, 13H), 1.28-1.01 (m, 2H).

### Step 6: Preparation of (R)-3-((5-acetyl-1-(6-aminohexan-2-yl)-7-(dimethylcarbamoyl)-1H-benzo[d]imidazol-2-yl)carbamoyl)benzoic acid

5 M HCl (5.0 ml) was added to a solution of tert-butyl (R)-3-((5-acetyl-1-(6-((tert-butoxycarbonyl)amino)hexan-2-yl)-7-(dimethylcarbamoyl)-1H-benzo[d]imidazol-2-yl)carbamoyl)benzoate (51 mg, 77%) in dioaxane (5.0 ml). The reaction mixture was stirred for 48 h at rt and then concentrated under reduced pressure. The product was used without further purification.

### Step 7: Preparation of (R)-1⁵-acetyl-N,N,11-trimethyl-3,5-dioxo-1¹H-2,6-diaza-1(2,1)-benzo[d]imidazola-4(1,3)-benzenacycloundecaphane-1⁷-carboxamide

N,N-diisopropylethylamine (68 μl, 393 μmol) and HATU (45 mg, 118 μmol) were added to a suspension of (R)-3-((5-acetyl-1-(6-aminohexan-2-yl)-7-(dimethylcarbamoyl)-1H-benzo[d]imidazol-2-yl)carbamoyl)benzoic acid (38 mg, 78 μmol) in THF (160 ml). The reaction mixture was stirred for 5 days at rt. The reaction mixture was concentrated to dryness and purified using preparative HPLC (15-45% MeCN/H₂O with 0.1% TFA) to give (R)-1⁵-acetyl-N,N,11-trimethyl-3,5-dioxo-1¹H-2,6-diaza-1(2,1)-benzo[d]imidazola-4(1,3)-benzenacycloundecaphane-1⁷-carboxamide (13 mg, 34%).

### Step 8: Preparation of (R)-15-(2-hydroxypropan-2-yl)-N,N,11-trimethyl-3,5-dioxo-1¹H-2,6-diaza-1(2,1)-benzo[d]imidazola-4(1,3)-benzenacycloundecaphane-1⁷-carboxamide

1.6 M MeLi in THF (67 μl, 107 μmol) was added to a solution of (R)-1⁵-acetyl-N,N,11-trimethyl-3,5-dioxo-1¹H-2,6-diaza-1(2,1)-benzo[d]imidazola-4(1,3)-benzenacycloundecaphane-1⁷-carboxamide in THF (3.0 ml) at −78° C. The reaction was stirred at −78° C. for min and then allowed to reach 0° C. over 5 min and then quenched with AcOH (100 μl). The reaction mixture was concentrated under reduced pressure and purified using preparative HPLC (10-30% MeCN/H₂O with 0.1% TFA) to give (R)-1⁵-(2-hydroxypropan-2-yl)-N,N,11-trimethyl-3,5-dioxo-1¹H-2,6-diaza-1(2,1)-benzo[d]imidazola-4(1,3)-benzenacycloundecaphane-1⁷-carboxamide (3 mg, 22%).

¹H NMR (CDCl₃) δ: 11.87 (s, 1H), 9.19-8.89 (m, 1H), 8.28 (d, 1H), 7.96 (d, 1H), 7.60 (m, 1H), 7.49 (d, 1H), 7.24 (d, 1H), 5.95-5.54 (m, 1H), 3.90 (s, 1H), 3.38-3.08 (m, 5H), 3.04-2.93 (m, 3H), 2.20-1.68 (m, 6H), 1.64 (s, 8H), 1.41-1.04 (m, 4H);

LC/MS: M+H=492.0.

### General Method B:

To a solution of corresponding acid (1 eq), amine (1.5 eq) in MeCN and DMF was added TCFH (2 eq.) and 1-methyl-1H-imidazole (5 eq.) at 0° C. The reaction mixture was stirred at rt until reaction was completed. The reaction mixture was concentrated under reduced pressure to afford and the product was purified by preparative HPLC.

### Examples 70 and 71: (E)-N-(2-fluoroethyl)-N,11-dimethyl-3,5-dioxo-1²,1³-dihydro-1¹H-2,6-diaza-1(2,1)-benzo[d]imidazola-4(1,3)-benzenacycloundecaphane-1⁷-carboxamide

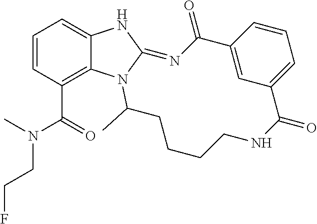 [SMILES: CC1CCCCNC(=O)c2cccc(c2)C(=O)/N=C2\Nc3cccc(C(=O)N(C)CCF)c3N21]

Prepared according to general method B starting from (E)-11-methyl-3,5-dioxo-1²,1³-dihydro-1¹H-2,6-diaza-1(2,1)-benzo[d]imidazola-4(1,3)-benzenacycloundecaphane-1⁷-carboxylic acid and 2-fluoro-N-methylethan-1-amine. The enantiomers were separated using chiral chromatography.

### Example 70, Enantiomer 1

Chiralpak IG (4.6×250) 5 um, MeOH/EtOH=50:50 (v/v), Flow Rate: 1 ml/min, Rt=12.82 min.

¹H NMR (DMSO-d₆) @ 353.2K δ 8.84 (s, 1H), 8.07 (d, 1H), 7.62 (d, 1H), 7.49 (m, 1H), 7.34 (bs, 1H), 7.00 (m, 1H), 6.76 (d, 1H), 4.69 (m, 1H), 3.54 (m, 2H), 1.92 (m, 1H), 1.43 (m, 1H); LC/MS: M+H=466.20.

### Example 71, Enantiomer 2

Chiralpak IG (4.6×250) 5 um, MeOH/EtOH=50:50 (v/v), Flow Rate: 1 ml/min, Rt=16.90 min.

¹H NMR (DMSO-d₆) @ 353.2K δ 8.83 (s, 1H), 8.08 (d, 1H), 7.67 (d, 1H), 7.50 (m, 1H), 7.10 (bs, 1H), 6.89 (m, 1H), 4.69 (m, 1H), 3.54 (m, 2H), 1.92 (m, 1H), 1.43 (m, 1H); LC/MS: M+H=466.20.

### Examples 72 and 73: (E)-N-(2,2-difluoroethyl)-N,11-dimethyl-3,5-dioxo-1²,1³-dihydro-1¹H-2,6-diaza-1(2,1)-benzo[d]imidazola-4(1,3)-benzenacycloundecaphane-1⁷-carboxamide

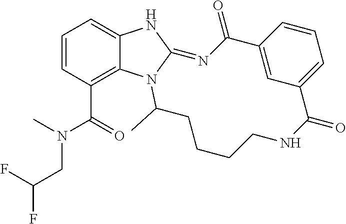 [SMILES: CC1CCCCNC(=O)c2cccc(c2)C(=O)/N=C2\Nc3cccc(C(=O)N(C)CC(F)F)c3N21]

Prepared according to general method B starting from (E)-11-methyl-3,5-dioxo-1²,1³-dihydro-1¹H-2,6-diaza-1(2,1)-benzo[d]imidazola-4(1,3)-benzenacycloundecaphane-1⁷-carboxylic acid and 2,2-difluoro-N-methylethan-1-amine. The enantiomers were separated using chiral chromatography.

### Example 72, Enantiomer 1

Chiralpak IG (4.6×250) 5 um, IPA/EtOAc=50:50 (v/v), Flow Rate: 1 ml/min, Rt=5.82 min

¹H NMR (DMSO-d₆) @ 353.2K δ 8.83 (s, 1H), 8.09 (d, 1H), 7.69 (d, 1H), 7.54 (m, 2H), 7.42 (bs, 1H), 7.17 (m, 1H), 6.97 (m, 1H), 6.39 (m, 1H), 3.53 (m, 1H), 1.92 (m, 1H), 1.60 (d, 3H), 1.45 (m, 1H); LC/MS: M+H=484.23.

### Example 73, Enantiomer 2

Chiralpak IG (4.6×250) 5 um, IPA/EtOAc=50:50 (v/v), Flow Rate: 1 ml/min, Rt=6.85 min

¹H NMR (DMSO-d₆) @ 353.2K δ 8.83 (s, 1H), 8.09 (d, 1H), 7.68 (d, 1H), 7.52 (m, 2H), 7.40 (bs, 1H), 7.12 (m, 1H), 6.90 (m, 1H), 6.33 (m, 1H), 3.53 (m, 1H), 1.92 (m, 1H), 1.60 (d, 3H), 1.45 (m, 1H); LC/MS: M+H=484.19.

### Example 74: (S,E)-11-methyl-1⁷-(3-methyl-2-oxoimidazolidin-1-yl)-1²,1³-dihydro-1H-2,6-diaza-1(2,1)-benzo[d]imidazola-4(1,3)-benzenacycloundecaphane-3,5-dione

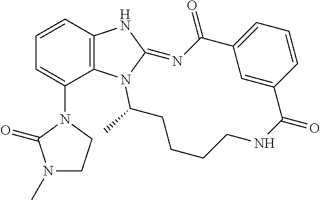 [SMILES: C[C@H]1CCCCNC(=O)c2cccc(c2)C(=O)/N=C2\Nc3cccc(N4CCN(C)C4=O)c3N21]

Example 74 was prepared in the same manner as Example 60: (R,E)-11-methyl-1⁷-(3-methyl-2-oxoimidazolidin-1-yl)-1²,1³-dihydro-1¹H-2,6-diaza-1(2,1)-benzo[d]imidazola-4(1,3)-benzenacycloundecaphane-3,5-dione but using tert-butyl (S)-(5-aminohexyl)carbamate instead of tert-butyl (R)-(5-aminohexyl)carbamate.

¹H NMR (DMSO-d₆) δ 12.50 (s, 1H), 8.79 (s, 1H), 8.11 (d, 1H), 7.79 (s, 1H), 7.74 (d, 1H), 7.59 (m, 1H), 7.48 (d, 1H), 7.22 (m, 1H), 7.15-7.06 (m, 1H), 5.06 (s, 1H), 3.87-2.90 (m, 7H), 2.79 (d, 3H), 1.90 (s, 3H), 1.66 (s, 3H), 1.58-1.33 (m, 2H); LC/MS: M+H=461.0.

### Examples 75 and 76: (E)-11-methyl-1⁷-((R)-3-methylmorpholine-4-carbonyl)-1²,1³-dihydro-1¹H-2,6-diaza-1(2,1)-benzo[d]imidazola-4(1,3)-benzenacycloundecaphane-3,5-dione

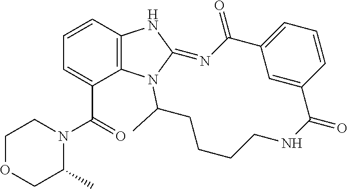 [SMILES: CC1CCCCNC(=O)c2cccc(c2)C(=O)/N=C2\Nc3cccc(C(=O)N4CCOC[C@H]4C)c3N21]

Prepared according to general method B starting from (E)-11-methyl-3,5-dioxo-1²,1³-dihydro-1¹H-2,6-diaza-1(2,1)-benzo[d]imidazola-4(1,3)-benzenacycloundecaphane-1⁷-carboxylic acid and (R)-3-methylmorpholine. The diastereomers were separated using chiral chromatography.

### Example 75, Diastereomer 1

Lux-Amylose-1 (4.6×250) 5 um, IPA/MeOH=50:50 (v/v), Flow Rate: 1 ml/min, Rt=8.30 min

¹H NMR (DMSO-d₆+D₂O) @ 353.2K δ 8.80 (s, 1H), 8.08 (d, 1H), 7.70 (d, 1H), 7.53 (m, 2H), 7.17 (m, 1H), 4.62 (m, 1H), 3.71 (m, 2H), 3.51 (m, 2H); LC/MS: M+H=490.37.

### Example 76, Diastereomer 2

Lux-Amylose-1 (4.6×250) 5 um, IPA/MeOH=50:50 (v/v), Flow Rate: 1 ml/min, Rt=9.71 min.

¹H NMR (DMSO-d₆+D₂O) @ 353.2K δ 8.77 (s, 1H), 8.06 (d, 1H), 7.66 (d, 1H), 7.47 (m, 2H), 7.10 (m, 1H), 4.61 (m, 1H), 3.71 (m, 2H); LC/MS: M+H=490.46.

### Examples 77 and 78: (E)-11-methyl-1⁷-((S)-3-methylmorpholine-4-carbonyl)-1²,1³-dihydro-1¹H-2,6-diaza-1(2,1)-benzo[d]imidazola-4(1,3)-benzenacycloundecaphane-3,5-dione

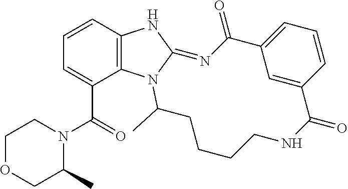 [SMILES: CC1CCCCNC(=O)c2cccc(c2)C(=O)/N=C2\Nc3cccc(C(=O)N4CCOC[C@@H]4C)c3N21]

Prepared according to general method B starting from (E)-11-methyl-3,5-dioxo-1²,1³-dihydro-1¹H-2,6-diaza-1(2,1)-benzo[d]imidazola-4(1,3)-benzenacycloundecaphane-1⁷-carboxylic acid and (S)-3-methylmorpholine. The diastereomers were separated using chiral chromatography.

### Example 77, Diastereomer 1

Chiralpak IG (4.6×250) 5 um, MeOH/EtOH=50:50 (v/v), Flow Rate: 1 ml/min, Rt=17.61 min.

¹H NMR (DMSO-d₆) @ 353.2K δ 8.85 (s, 1H), 8.10 (d, 1H), 7.74 (d, 1H), 7.57 (m, 2H), 7.48 (bs, 1H), 7.21 (m, 1H), 4.66 (m, 1H), 3.74 (m, 2H); LC/MS: M+H=490.46.

### Example 78, Diastereomer 2

Chiralpak IG (4.6×250) 5 μm, MeOH/EtOH=50:50 (v/v), Flow Rate: 1 ml/min, Rt=17.61 min.

¹H NMR (DMSO-d₆) @ 353.2K δ 8.81 (s, 1H), 8.03 (m, 1H), 7.56 (d, 1H), 7.45 (m, 1H), 7.37 (m, 1H), 7.36 (m, 1H), 6.92 (m, 1H); LC/MS: M+H=490.46.

### Examples 79 and 80: (E)-11-methyl-1⁷-(1-oxa-6-azaspiro[3.3]heptane-6-carbonyl)-1²,1³-dihydro-1¹H-2,6-diaza-1(2,1)-benzo[d]imidazola-4(1,3)-benzenacycloundecaphane-3,5-dione

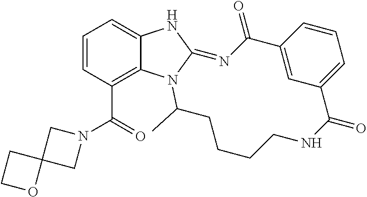 [SMILES: CC1CCCCNC(=O)c2cccc(c2)C(=O)/N=C2\Nc3cccc(C(=O)N4CC5(CCO5)C4)c3N21]

To a solution of (E)-11-methyl-3,5-dioxo-1²,1³-dihydro-1¹H-2,6-diaza-1(2,1)-benzo[d]imidazola-4(1,3)-benzenacycloundecaphane-1⁷-carboxylic acid (250 mg, 412 μmol) and bis(1-oxa-6-azaspiro[3.3]heptane); oxalate (178 mg, 618 μmol) in MeCN (8 mL) and DMF (2 mL) was added TCFH (231 mg, 824 μmol) and 1-methyl-1H-imidazole (164 L, 2.06 mmol) at 0° C. and stirred for 2 h. The reaction mixture was concentrated under reduced pressure. The residue was triturated with water, filtered, and concentrated. The crude product was purified by using prep HPLC to give 32 mg (E)-11-methyl-1⁷-(1-oxa-6-azaspiro[3.3]heptane-6-carbonyl)-1²,1³-dihydro-1¹H-2,6-diaza-1(2,1)-benzo[d]imidazola-4(1,3)-benzenacycloundecaphane-3,5-dione. The enantiomers were separated by chiral chromatography.

### Example 79, Enantiomer 1

Chiralpak IC (4.6×250) 5 μm, IPA/Ethyl acetate=80:20 (v/v), Flow Rate: 1 ml/min, Rt=7.87

¹H NMR @ 353.1 K (DMSO-d₆) δ: 8.82 (s, 3H), 8.08 (d, 1H), 7.73 (d, 1H), 7.57 (m, 2H), 7.23 (m, 1H), 7.10 (d, 1H), 4.84 (m, 1H), 4.36 (m, 3H), 3.11 (m, 1H), 1.88 (m, 3H), 1.57 (d, 3H); LC/MS: M+H=488.44.

### Example 80, Enantiomer 2

Chiralpak IC (4.6×250) 5 μm, IPA/Ethyl acetate=80:20 (v/v), Flow Rate: 1 ml/min, Rt=10.29

¹H NMR @ 353.1 K (DMSO-d₆+D₂O) δ: 8.87 (s, 3H), 8.11 (d, 1H), 7.74 (d, 1H), 7.56 (m, 2H), 7.23 (m, 1H), 7.11 (d, 1H), 4.90 (m, 1H), 4.34 (m, 3H), 2.83 (m, 1H), 1.89 (m, 3H), 1.59 (d, 3H); LC/MS: M+H=488.44.

### Examples 81 and 82: (E)-1⁷-(3-methoxyazetidine-1-carbonyl)-11-methyl-1²,1³-dihydro-1¹H-2,6-diaza-1(2,1)-benzo[d]imidazola-4(1,3)-benzenacycloundecaphane-3,5-dione

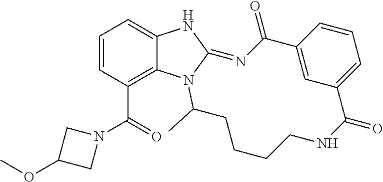 [SMILES: COC1CN(C(=O)c2cccc3c2N2/C(=N/C(=O)c4cccc(c4)C(=O)NCCCCC2C)N3)C1]

To a solution of (E)-11-methyl-3,5-dioxo-12,13-dihydro-11H-2,6-diaza-1(2,1)-benzo[d]imidazola-4(1,3)-benzenacycloundecaphane-1⁷-carboxylic acid (150 mg, 369 μmol) and 3-methoxyazetidine (39 mg, 443 μmol) in MeCN (3 mL) and DMF (0.5 mL) was added TCFH (207 mg, 738 μmol) and 1-methyl-1H-imidazole (147 μL, 1.85 mmol) at 0° C. and stirred for 2 h. The reaction mixture was concentrated under reduced pressure. The residue was triturated with water, filtered, and concentrated. The crude product was purified by using prep HPLC to give 32 mg racemic (E)-1⁷-(3-methoxyazetidine-1-carbonyl)-11-methyl-1²,1³-dihydro-1¹H-2,6-diaza-1(2,1)-benzo[d]imidazola-4(1,3)-benzenacycloundecaphane-3,5-dione. The enantiomers were separated by chiral chromatography.

### Example 81, Enantiomer 1

Chiralpak IC (4.6×250) 5 um, 0.1% TEA in n-Hexane/EtOH=20:80 (v/v), Flow Rate: 1 ml/min, Rt=12.60

¹H NMR @ 352.9 K (DMSO-d₆) δ: 8.90 (s, 3H), 8.11 (d, 1H), 7.75 (d, 1H), 7.58 (m, 2H), 7.51 (m, 1H), 7.23 (m, 1H), 7.12 (d, 1H), 4.93 (m, 1H), 4.284 (m, 3H), 4.13 (m, 1H), 3.91 (m, 1H), 3.45 (m, 1H), 3.24 (s, 3H), 1.91 (m, 3H), 1.64 (d, 3H); LC/MS: M+H=476.41.

### Example 82, Enantiomer 2

Chiralpak IC (4.6×250) 5 μm, 0.1% TEA in n-Hexane/EtOH=20:80 (v/v), Flow Rate: 1 ml/min, Rt=15.08

¹H NMR @ 352.7 K (DMSO-d₆) δ: 8.90 (s, 3H), 8.11 (d, 1H), 7.75 (d, 1H), 7.58 (m, 2H), 7.51 (m, 1H), 7.23 (m, 1H), 7.12 (d, 1H), 4.93 (m, 1H), 4.284 (m, 3H), 4.13 (m, 1H), 3.91 (m, 1H), 3.45 (m, 1H), 3.24 (s, 3H), 1.91 (m, 3H), 1.64 (d, 3H); LC/MS: M+H=476.41.

### Examples 83 and 84: (R,E)-11-methyl-1⁷-(1-methyl-1H-1,2,4-triazol-3-yl)-1²,1³-dihydro-1¹H-2,6-diaza-1(2,1)-benzo[d]imidazola-4(1,3)-benzenacycloundecaphane-3,5-dione

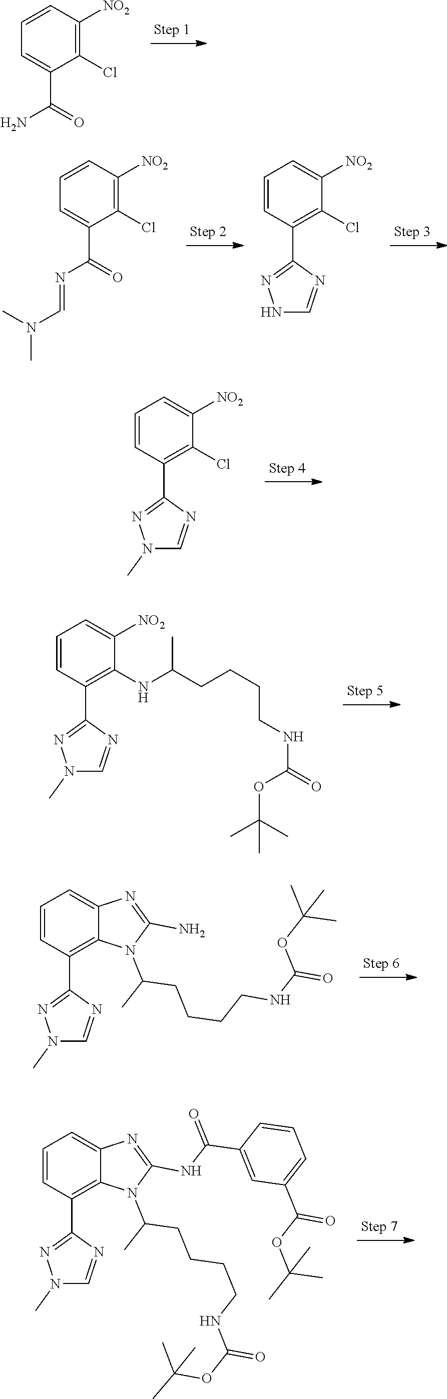

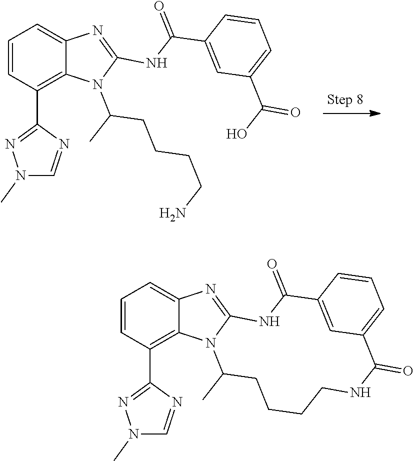 [SMILES: CC(CCCCN)n1c(NC(=O)c2cccc(C(=O)O)c2)nc2cccc(-c3ncn(C)n3)c21 ; CC1CCCCNC(=O)c2cccc(c2)C(=O)Nc2nc3cccc(-c4ncn(C)n4)c3n21]

### Scheme: Synthesis

### Step 1: Preparation of (E)-2-chloro-N-((dimethylamino)methylene)-3-nitrobenzamide

A slurry of 2-chloro-3-nitrobenzamide (277 mg, 1.38 mmol) in dimethoxy-N,N-dimethylmethaneamine (1.0 ml) was heated until starting material was dissolved. Heating was continued with removal of MeOH through distillation for 4 h. Heptane (5 ml) was added to the reaction mixture and the resulting mixture was cooled to rt. The precipitate was collected by filtration, and dried to give (E)-2-chloro-N-((dimethylamino)methylene)-3-nitrobenzamide (334 mg, 95%).

¹H NMR (DMSO-d₆) δ: 8.63 (s, 1H), 8.03 (d, 1H), 7.93 (d, 1H), 7.61 (m, 1H), 3.23 (s, 3H), 3.07 (s, 3H).

### Step 2: Preparation of 3-(2-chloro-3-nitrophenyl)-1H-1,2,4-triazole

Hydrazine (0.2 ml) was added to a solution of (E)-2-chloro-N-((dimethylamino)methylene)-3-nitrobenzamide (329 mg, 1.29 mmol) in acetic acid (6.0 ml) cooled with an ice bath. Dropwise addition of hydrazine (1.8 ml) at while cooling. The formed slurry was allowed to reach room temperature and then added to 1 M NaOH (40 ml). The mixture was extracted with EtOAc (3×20 ml). The combined organic fractions were dried and concentrated to give 3-(2-chloro-3-nitrophenyl)-1H-1,2,4-triazole (289 mg, 99%) which was used without further purification.

¹H NMR (DMSO-d₆) δ: 8.69 (s, 1H), 8.13 (m, 1H), 8.09 (m, 1H), 7.70 (m, 1H).

### Step 3: Preparation of 3-(2-chloro-3-nitrophenyl)-1-methyl-1H-1,2,4-triazole

Iodomethane (104 μl, 1.67 mmol) was added to a solution of 3-(2-chloro-3-nitrophenyl)-1H-1,2,4-triazole (289 mg, 1.29 mmol) and K₂CO₃ (533 mg, 3.86 mmol) in DMF (2.5 ml) at 0° C. The resulting reaction mixture was stirred at rt for 3.5 h. The mixture was diluted with water (30 mL) and extracted with EtOAc (3×20 mL). The combined organic layers were washed with brine (2×20 mL), dried and concentrated. The crude product was purified by reverse phase chromatography (C18 flash cartridge, 5-50% MeCN in water with 0.1% TFA) to give 3-(2-chloro-3-nitrophenyl)-1-methyl-1H-1,2,4-triazole (190 mg, 62%) and 5-(2-chloro-3-nitrophenyl)-1-methyl-1H-1,2,4-triazole (52.5 mg, 17%).

¹H NMR (CDCl₃) δ: 8.16 (s, 1H), 8.08 (m, 1H), 7.75 (m, 1H), 7.47 (m, 1H), 4.04 (s, 3H).

### Step 4: Preparation of tert-butyl (5-((2-(1-methyl-1H-1,2,4-triazol-3-yl)-6-nitrophenyl)amino)hexyl)carbamate

A solution of 3-(2-chloro-3-nitrophenyl)-1-methyl-1H-1,2,4-triazole (190 mg, 795 μmol) in MeCN (5.0 ml) was added to tert-butyl-(5-aminohexyl)carbamate (206 mg, 954 μmol) at rt, followed by K₂CO₃ (330 mg, 2.39 mmol). The resulting solution was heated at 80° C. for 6 days. The reaction mixture was cooled to rt, filtered and the filtrate was concentrated under reduced pressure. The crude product was purified by reverse phase chromatography (C18 flash cartridge, 20-70% MeCN in water with 0.1% TFA) to give tert-butyl (5-((2-(1-methyl-1H-1,2,4-triazol-3-yl)-6-nitrophenyl)amino)hexyl)carbamate (296.8 mg, 89%).

LC/MS: M+H=419.0. Rt=2.90 min

### Step 5: Preparation of tert-butyl (5-(2-amino-7-(1-methyl-1H-1,2,4-triazol-3-yl)-1H-benzo[d]imidazol-1-yl)hexyl)carbamate

To a solution of tert-butyl (5-((2-(1-methyl-1H-1,2,4-triazol-3-yl)-6-nitrophenyl)amino)hexyl)carbamate (297 mg, 709 μmol) in MeOH (15 ml) under N₂, was added 10% Pd/C (38 mg, 36 μmol) and the mixture was hydrogenated at rt for 1.5 h. The mixture was evacuated, filtered through a syringe filter with MeOH. The filtrate was concentrated under reduced pressure. The residue was dissolved in MeOH (5.0 ml) and water (1.0 ml) and CNBr (91 mg, 851 μmol) was added to the solution at rt for 36 h. The reaction mixture was concentrated to dryness under reduced pressure. The residue was purified by flash chromatography (silica gel, 1-7% MeOH in CH₂Cl₂ with 1% NH₃ (28% aq.)) to give tert-butyl (5-(2-amino-7-(1-methyl-1H-1,2,4-triazol-3-yl)-1H-benzo[d]imidazol-1-yl)hexyl)carbamate (140 mg, 47%).

LC/MS: M+H=414.0. Rt=1.74 min

### Step 6: Preparation of tert-butyl 3-((1-(6-((tert-butoxycarbonyl)amino)hexan-2-yl)-7-(1-methyl-1H-1,2,4-triazol-3-yl)-1H-benzo[d]imidazol-2-yl)carbamoyl)benzoate

A solution of HATU (154 mg, 406 μmol) and 3-(tert-butoxycarbonyl)benzoic acid (90 mg, 406 μmol) in MeCN (5.0 ml) was added to a solution of tert-butyl (5-(2-amino-7-(1-methyl-1H-1,2,4-triazol-3-yl)-1H-benzo[d]imidazol-1-yl)hexyl)carbamate (140 mg, 339 μmol) and N,N-diisopropylethylamine (175 μl, 1.02 mmol) to give a yellow solution. The reaction mixture was stirred overnight and then concentrated. The crude product was purified by flash chromatography (silica gel, 10-50% EtOAc in pet. ether) to give tert-butyl 3-((1-(6-((tert-butoxycarbonyl)amino)hexan-2-yl)-7-(1-methyl-1H-1,2,4-triazol-3-yl)-1H-benzo[d]imidazol-2-yl)carbamoyl)benzoate (188 mg, 90%).

LC/MS: M+H=618.3. Rt=3.37 min

### Step 7: Preparation of 3-((1-(6-aminohexan-2-yl)-7-(1-methyl-1H-1,2,4-triazol-3-yl)-1H-benzo[d]imidazol-2-yl)carbamoyl)benzoic acid

A solution of tert-butyl 3-((1-(6-((tert-butoxycarbonyl)amino)hexan-2-yl)-7-(1-methyl-1H-1,2,4-triazol-3-yl)-1H-benzo[d]imidazol-2-yl)carbamoyl)benzoate in 4M HCl in dioxane (15 ml) was stirred at rt for 5 h. Volatiles were removed under reduced pressure and the product was used without further purification

LC/MS: M+H=462.0. Rt=1.75 min

### Step 8: Preparation of (R)-11-methyl-1⁷-(1-methyl-1H-1,2,4-triazol-3-yl)-1¹H-2,6-diaza-1(2,1)-benzo[d]imidazola-4(1,3)-benzenacycloundecaphane-3,5-dione

HATU (174 mg, 458 μmol) was added to a solution of 3-((1-(6-aminohexan-2-yl)-7-(1-methyl-1H-1,2,4-triazol-3-yl)-1H-benzo[d]imidazol-2-yl)carbamoyl)benzoic acid (152 mg, 305 μmol) and N,N-diisopropylethylamine (175 μl, 1.02 mmol) in THF (610 ml). The reaction mixture was heated to 50° C. and stirred for 2 days. The mixture was concentrated to dryness. The residue was purified by reverse phase chromatography C18 column, 20-50% MeCN in water containing 0.1% formic acid) to give 28 mg of racemic 11-methyl-1⁷-(1-methyl-1H-1,2,4-triazol-3-yl)-1¹H-2,6-diaza-1(2,1)-benzo[d]imidazola-4(1,3)-benzenacycloundecaphane-3,5-dione. The solids were dissolved in CH₂Cl₂ (1.5 mL) and purified by preparative chiral HPLC an IA column (3% EtOAc in CH₂Cl₂) to give:

### Example 83, Enantiomer 1

(R)-11-methyl-1⁷-(1-methyl-1H-1,2,4-triazol-3-yl)-1¹H-2,6-diaza-1(2,1)-benzo[d]imidazola-4(1,3)-benzenacycloundecaphane-3,5-dione (10.1 mg, 7.4%)

¹H NMR (DMSO-d₆) δ: 12.75 (s, 1H), 8.77 (s, 1H), 8.66 (s, 1H), 8.11 (d, 1H), 7.80-7.70 (m, 2H), 7.63 (d, 1H), 7.59 (m, 1H), 7.32 (d, J=7.5 Hz, 1H), 7.26 (m, 1H), 4.84-4.47 (m, 1H), 3.98 (s, 3H), 3.65-3.48 (m, 1H), 3.07-2.93 (m, 1H), 1.98-1.66 (m, 3H), 1.62-1.20 (m, 6H); LC/MS: M+H=444.0.

### Example 84, Enantiomer 2

(S)-11-methyl-1⁷-(1-methyl-1H-1,2,4-triazol-3-yl)-1¹H-2,6-diaza-1(2,1)-benzo[d]imidazola-4(1,3)-benzenacycloundecaphane-3,5-dione (11.4 mg, 8.4%)

¹H NMR (DMSO-d₆) δ: 12.76 (s, 1H), 8.77 (s, 1H), 8.66 (s, 1H), 8.11 (d, 1H), 7.81-7.68 (m, 2H), 7.63 (d, 1H), 7.59 (m, 1H), 7.32 (d, J=7.4 Hz, 1H), 7.26 (m, 1H), 4.84-4.44 (m, 1H), 3.98 (s, 3H), 3.70-3.46 (m, 1H), 3.10-2.92 (m, 1H), 1.98-1.67 (m, 3H), 1.64-1.20 (m, 6H); LC/MS: M+H=444.0

### Examples 85 and 86: (R,E)-11-methyl-1⁷-(1-methyl-1H-1,2,4-triazol-5-yl)-1²,1³-dihydro-1¹H-2,6-diaza-1(2,1)-benzo[d]imidazola-4(1,3)-benzenacycloundecaphane-3,5-dione

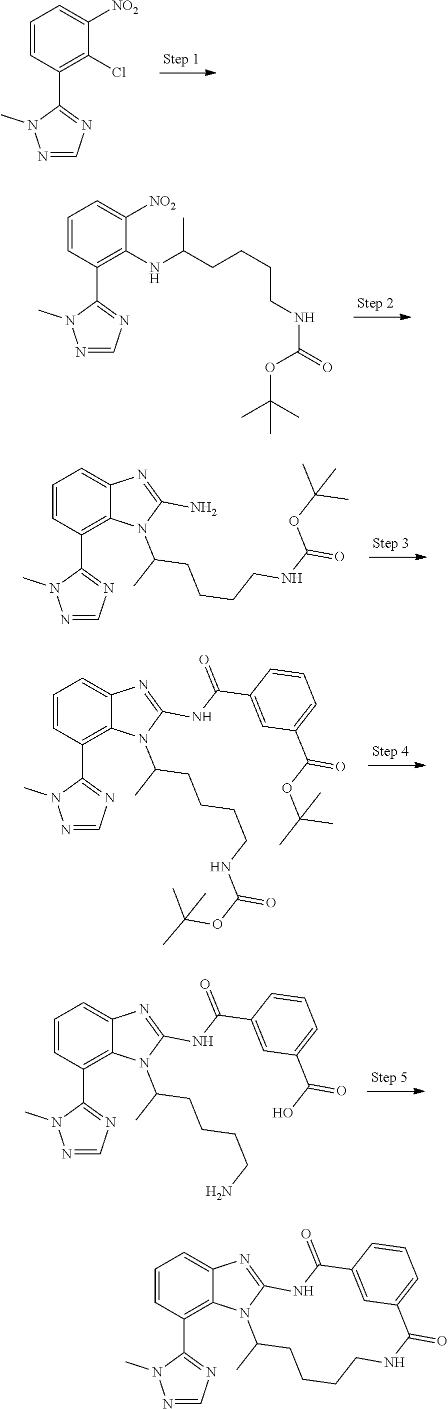

### Step 1: Preparation of tert-butyl (5-((2-(1-methyl-1H-1,2,4-triazol-5-yl)-6-nitrophenyl)amino)hexyl)carbamate

A solution of 5-(2-chloro-3-nitrophenyl)-1-methyl-1H-1,2,4-triazole (52 mg, 220 μmol) in MeCN (5.0 ml) was added to tert-butyl-(5-aminohexyl)carbamate (57 mg, 264 μmol) at rt, followed by K₂CO₃ (91 mg, 660 μmol). The resulting solution was heated at 80° C. for 7 days. The reaction mixture was cooled to rt, filtered and the filtrate was concentrated under reduced pressure. The crude product was purified by reverse phase chromatography (C18 flash cartridge, 20-70% MeCN in water with 0.1% TFA) to give tert-butyl (5-((2-(1-methyl-1H-1,2,4-triazol-5-yl)-6-nitrophenyl)amino)hexyl)carbamate (75 mg, 81%).

LC/MS: M+H=419.0. Rt=2.75 min

### Step 2: Preparation of tert-butyl (5-(2-amino-7-(1-methyl-1H-1,2,4-triazol-5-yl)-1H-benzo[d]imidazol-1-yl)hexyl)carbamate

To a solution of tert-butyl (5-((2-(1-methyl-1H-1,2,4-triazol-5-yl)-6-nitrophenyl)amino)hexyl)carbamate (75 mg, 178 μmol) in MeOH (15 ml) under N₂, was added 10% Pd/C (9 mg, 9 μmol) and the mixture was hydrogenated at rt for 1.5 h. The mixture was evacuated, filtered through a syringe filter with MeOH. The filtrate was concentrated under reduced pressure. The residue was dissolved in MeOH (5.0 ml) and water (1.0 ml) and CNBr (23 mg, 213 μmol) was added to the solution at rt for 36 h. The reaction mixture was concentrated to dryness under reduced pressure. The residue was purified by flash chromatography (silica gel, 2-5% MeOH in CH₂Cl₂ with 1% NH₃ (28% aq.)) to give tert-butyl (5-(2-amino-7-(1-methyl-1H-1,2,4-triazol-5-yl)-1H-benzo[d]imidazol-1-yl)hexyl)carbamate (59 mg, 79%).

LC/MS: M+H=414.0. Rt=1.72 min.

### Step 3: Preparation of tert-butyl 3-((1-(6-((tert-butoxycarbonyl)amino)hexan-2-yl)-7-(1-methyl-1H-1,2,4-triazol-5-yl)-1H-benzo[d]imidazol-2-yl)carbamoyl)benzoate

A solution of 3-(tert-butoxycarbonyl)benzoic acid (38 mg, 170 μmol) and HATU (65 mg, 170 μmol) in MeCN (5.0 ml) was added to a solution of tert-butyl (5-(2-amino-7-(1-methyl-1H-1,2,4-triazol-5-yl)-1H-benzo[d]imidazol-1-yl)hexyl)carbamate (59 mg, 141 μmol) and N,N-diisopropylethylamine (73 μl, 424 μmol) in MeCN (5 ml) at rt. The resulting solution was stirred overnight. The reaction mixture was concentrated under reduced pressure and purified using flash chromatography (SiO₂, 20-80% EtOAc in pet. ether) to give tert-butyl 3-((1-(6-((tert-butoxycarbonyl)amino)hexan-2-yl)-7-(1-methyl-1H-1,2,4-triazol-5-yl)-1H-benzo[d]imidazol-2-yl)carbamoyl)benzoate (56 mg, 64%).

LC/MS: M+H=618.30. Rt=3.38 min.

### Step 4: Preparation of 3-((1-(6-aminohexan-2-yl)-7-(1-methyl-1H-1,2,4-triazol-5-yl)-1H-benzo[d]imidazol-2-yl)carbamoyl)benzoic acid

4 M HCl in dioxane (10.0 ml) was added to tert-butyl 3-((1-(6-((tert-butoxycarbonyl)amino)hexan-2-yl)-7-(1-methyl-1H-1,2,4-triazol-5-yl)-1H-benzo[d]imidazol-2-yl)carbamoyl)benzoate (56 mg, 91 μmol). The reaction mixture was stirred at rt for 5 h. The reaction mixture was concentrated to dryness under reduced pressure. The product was use without further purification.

### Step 5: Preparation of 11-methyl-1⁷-(1-methyl-1H-1,2,4-triazol-5-yl)-1¹H-2,6-diaza-1(2,1)-benzo[d]imidazola-4(1,3)-benzenacycloundecaphane-3,5-dione

HATU (52 mg, 136 μmol) was added to a solution of 3-((1-(6-aminohexan-2-yl)-7-(1-methyl-1H-1,2,4-triazol-5-yl)-1H-benzo[d]imidazol-2-yl)carbamoyl)benzoic acid (45 mg, 91 μmol) in THF (181 ml). The mixture was heated at 50° C. overnight. The mixture was concentrated to dryness under reduced pressure. The residue was dissolved in DMSO and purified using reverse phase chromatography (20-50% MeCN in water containing 0.1% formic acid) to give racemic 11-methyl-1⁷-(1-methyl-1H-1,2,4-triazol-5-yl)-1¹H-2,6-diaza-1(2,1)-benzo[d]imidazola-4(1,3)-benzenacycloundecaphane-3,5-dione (10 mg). The racemic product was purified using chiral chromatography (HPLC IA column, 3% EtOAc in CH₂Cl₂) to give:

### Example 85, Enantiomer 1

(R,E)-11-methyl-1⁷-(1-methyl-1H-1,2,4-triazol-5-yl)-1¹H-2,6-diaza-1(2,1)-benzo[d]imidazola-4(1,3)-benzenacycloundecaphane-3,5-dione (3.4 mg, 8%); Rt=12.7 min

¹H NMR (DMSO-d₆) δ: 12.85 (s, 1H), 8.77 (s, 1H), 8.17 (s, 1H), 8.11 (d, 1H), 7.74 (m, 3H), 7.59 (m, 1H), 7.35 (m, 1H), 7.24 (d, 1H), 3.75 (s, 3H), 1.91-1.71 (m, 3H), 1.65-1.16 (m, 6H). LC/MS: M+H=444.0.

### Example 86, Enantiomer 2

(S,E)-11-methyl-1⁷-(1-methyl-1H-1,2,4-triazol-5-yl)-1¹H-2,6-diaza-1(2,1)-benzo[d]imidazola-4(1,3)-benzenacycloundecaphane-3,5-dione (4.5 mg, 11%); Rt=15.9 min

¹H NMR (DMSO-d₆) δ: 12.81 (s, 1H), 8.77 (s, 1H), 8.17 (s, 1H), 8.11 (d, 1H), 7.74 (m, 3H), 7.59 (m, 1H), 7.35 (m, 1H), 7.24 (d, 1H), 3.75 (s, 3H), 1.93-1.71 (m, 2H), 1.69-1.18 (m, 6H). LC/MS: M+H=444.0.

### Example 87: (S,E)-11-methyl-1⁷-(1-methyl-1H-1,2,3-triazol-4-yl)-1²,1³-dihydro-1¹H-2,6-diaza-1(2,1)-benzo[d]imidazola-4(1,3)-benzenacycloundecaphane-3,5-dione

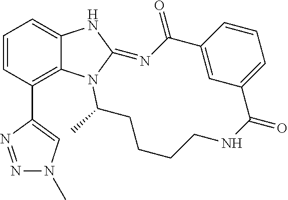 [SMILES: C[C@H]1CCCCNC(=O)c2cccc(c2)C(=O)/N=C2\Nc3cccc(-c4cn(C)nn4)c3N21]

Example 87 was prepared in the same manner as Example 62: (R,E)-11-methyl-1⁷-(1-methyl-1H-1,2,3-triazol-4-yl)-1²,1³-dihydro-1¹H-2,6-diaza-1(2,1)-benzo[d]imidazola-4(1,3)-benzenacycloundecaphane-3,5-dione but using tert-butyl (S)-(5-aminohexyl)carbamate instead of tert-butyl (R)-(5-aminohexyl)carbamate.

¹H NMR (CHCl₃-d) δ: 11.93 (s, 1H), 8.97 (s, 1H), 8.28 (d, 1H), 7.93 (d, 1H), 7.73 (s, 1H), 7.59 m, 1H), 7.39 (d1H), 7.24 (d, 1H), 7.16-7.06 (m, 1H), 6.29 (s, 1H), 4.75-4.47 (m, 1H), 4.27 (s, 3H), 3.97-3.77 (m, 1H), 3.17 (s, 2H), 2.19-1.99 (m, 1H), 1.88 (s, 2H), 1.56 (d, 4H), 1.43 (d1H); LC/MS: M+H=444.0.

### Examples 88 and 89: (E)-1⁷-((S)-3-fluoropyrrolidine-1-carbonyl)-11-methyl-1²,1³-dihydro-1¹H-2,6-diaza-1(2,1)-benzo[d]imidazola-4(1,3)-benzenacycloundecaphane-3,5-dione

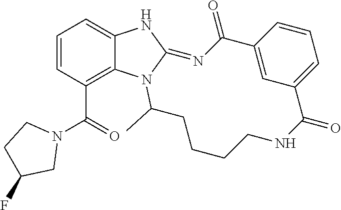 [SMILES: CC1CCCCNC(=O)c2cccc(c2)C(=O)/N=C2\Nc3cccc(C(=O)N4CC[C@H](F)C4)c3N21]

To a solution of (E)-11-methyl-3,5-dioxo-1²,1³-dihydro-1¹H-2,6-diaza-1(2,1)-benzo[d]imidazola-4(1,3)-benzenacycloundecaphane-1⁷-carboxylic acid (250 mg, 412 μmol) and (3S)-3-fluoropyrrolidine hydrochloride (77.6 mg, 618 μmol) in MeCN (5 ml) and DMF (1 ml) was added 1-methyl-1H-imidazole (164 mg, 2.06 mmol) and HATU (231 mg, 824 μmol) at rt. The reaction mixture was stirred at rt for 16 h. The reaction mixture was concentrated under reduced pressure. The crude product was purified by reverse phase column chromatography to get 24 mg of diastereomeric product. The diastereomers were separated using chiral separation.

### Example 88, Diastereomer 1

¹H NMR (DMSO-d₆) δ: 8.88 (s, 1H), 8.10 (d, 1H), 7.75 (d, 1H), 7.59 (m, 1H), 7.50 (m, 1H), 7.25 (m, 2H), 7.12 (m, 1H), 5.36 (m, 1H), 4.80 (m, 1H), 1.60 (d, 3H);

LC/MS: M+H=478.39.

Chiralpak-IE (4.6×250) 5 μm, Mobile Phase: 3% EtOH in CH₂Cl₂ (v/v) Flow Rate: 1 ml/min), Rt=30.70 min.

### Example 89, Diastereomer 2

¹H NMR (DMSO-d₆) @ 353.2K δ: 8.87 (s, 1H), 8.10 (d, 1H), 7.75 (d, 1H), 7.59 (m, 1H), 7.50 (m, 1H), 7.25 (m, 2H), 7.12 (m, 1H), 5.36 (m, 1H), 4.80 (m, 1H), 1.62 (d, 3H); LC/MS: M+H=478.39.

Chiralpak-IE (4.6×250) 5 um, Mobile Phase: 3% EtOH in CH₂Cl₂ (v/v) Flow Rate: 1 ml/min), Rt=35.27 min.

### Examples 90 and 91: (E)-11-methyl-1⁷-(1-methyl-1H-imidazol-2-yl)-1²,1³-dihydro-1¹H-2,6-diaza-1(2,1)-benzo[d]imidazola-4(1,3)-benzenacycloundecaphane-3,5-dione

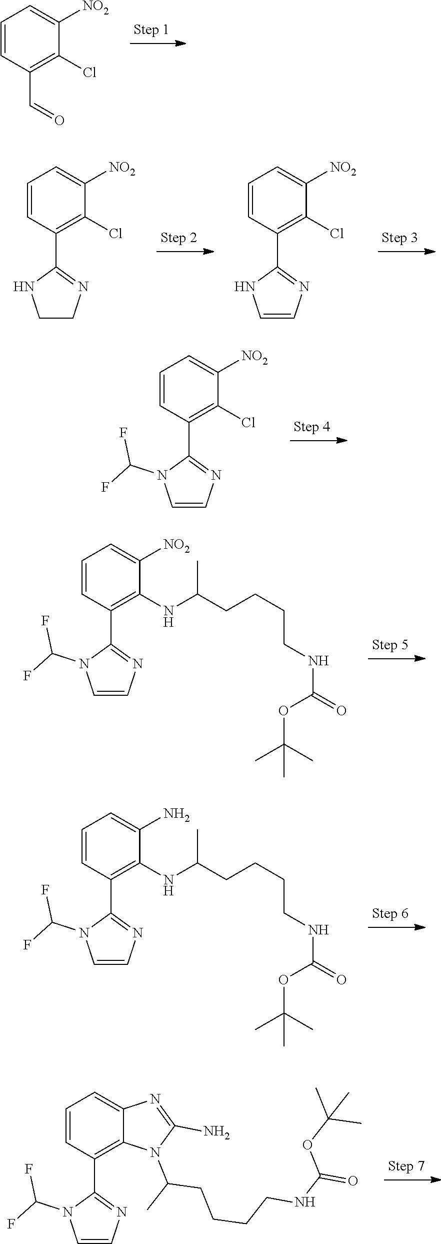

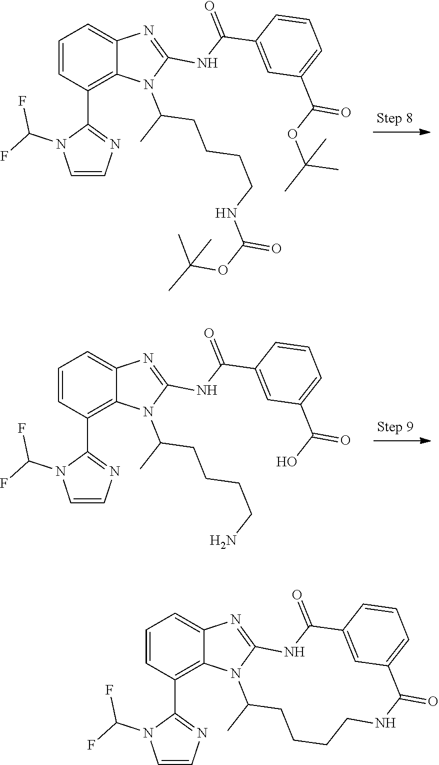 [SMILES: CC(CCCCNC(=O)OC(C)(C)C)n1c(NC(=O)c2cccc(C(=O)OC(C)(C)C)c2)nc2cccc(-c3nccn3C(F)F)c21 ; CC(CCCCN)n1c(NC(=O)c2cccc(C(=O)O)c2)nc2cccc(-c3nccn3C(F)F)c21 ; CC1CCCCNC(=O)c2cccc(c2)C(=O)Nc2nc3cccc(-c4nccn4C(F)F)c3n21]

### Step 1: Preparation of 2-(2-chloro-3-nitrophenyl)-4,5-dihydro-1H-imidazole

To a solution of 2-chloro-3-nitrobenzaldehyde (5.00 g, 26.9 mmol) in t-butanol (100 ml) was added ethane-1,2-diamine (1.98 mL, 29.6 mmol). The reaction mixture was stirred for 1 h at rt. Then K₂CO₃ (11.2 g, 80.8 mmol) and iodine (8.21 g, 32.3 mmol) was added. The reaction mixture was stirred at 70° C. for 6 h. The reaction mixture was filtered through celite and the filtrate was concentrated under reduced pressure. The residue was acidified with 3 M HCl and the solution was washed with EtOAc. The aqueous phase was basified with Na₂CO₃ and extracted with 10% MeOH/CH₂Cl₂. The organic phase was concentrated under reduced pressure to give crude 2-(2-chloro-3-nitrophenyl)-4,5-dihydro-1H-imidazole (3.0 g, 46%) which was used without further purification.

¹H NMR (DMSO-d₆) δ: 8.08 (d, 1H), 7.79 (d, 1H), 7.63 (m, 1H), 6.99 (s, 1H), 3.82 (m, 2H), 3.44 (m, 2H); LC/MS: M+H=226.03.

### Step 2: Preparation of 2-(2-chloro-3-nitrophenyl)-1H-imidazole

To a solution of 2-(2-chloro-3-nitrophenyl)-4,5-dihydro-1H-imidazole (1.0 g, 4.25 mmol) in DMSO (10.0 ml) was added K₂CO₃ (647 mg, 4.68 mmol) and iodobenzene diacetate (1.37 g, 4.25 mmol). The reaction mixture was stirred at rt for 3 h. The reaction was quenched with water and extracted with 10% MeOH/CH₂Cl₂. The organic layer was concentrated under reduced pressure and the residue was purified by flash chromatography (SiO2, 80% EtOAc/heptane) to give 2-(2-chloro-3-nitrophenyl)-1H-imidazole (951 mg, 44%).

¹H NMR (DMSO-d₆) δ: 8.03 (m, 21H), 7.679 (m, 1H), 7.25 (m, 2H); LC/MS: M+H=224.26.

### Step 3: Preparation of 2-(2-chloro-3-nitrophenyl)-1-(difluoromethyl)-1H-imidazole

To a solution of 2-(2-chloro-3-nitrophenyl)-1H-imidazole (2.0 g, 8.68 mmol) in MeCN (20.0 ml) KF (1.01 g, 17.4 mmol) and diethyl (bromodifluoromethyl)phosphonate (2.55 g, 9.54 mmol). The reaction mixture was stirred at rt for 16 h. The reaction was quenched with water and extracted with CH₂Cl₂. The organic layer was concentrated under reduced pressure and the crude product (1.8 g, 52%) was used without further purification.

LC/MS: M+H=274.08.

### Step 4: Preparation of tert-butyl (5-((2-(1-(difluoromethyl)-1H-imidazol-2-yl)-6-nitrophenyl)amino)hexyl)carbamate

K₂CO₃ (1.83 g, 13.2 mmol) was added to a solution of 2-(2-chloro-3-nitrophenyl)-1-(difluoromethyl)-1H-imidazole (1.5 g, 3.78 mmol) in MeCN (20.0 ml) at rt followed by tert-butyl (5-aminohexyl)carbamate (1.03 g, 4.54 mmol. The reaction mixture was heated at 80° C. for 16 h. The reaction mixture was diluted with water and extracted with EtOAc. The organic layer was concentrated under reduced pressure. The crude product was purified through flash chromatography (SiO2, 30% EtOAc\heptane) to give tert-butyl (5-((2-(1-(difluoromethyl)-1H-imidazol-2-yl)-6-nitrophenyl)amino)hexyl)carbamate (1.1 g, 54%).

¹H NMR (DMSO-d₆) δ: 8.25 (d, 1H), 7.56 (m, 1H), 7.27 (m, 2H), 6.99 (m, 1H), 2.79 (m, 2H), 2.23 (m, 1H), 1.36 (s, 9H, 1.19 (m, 3H), 0.87 (d, 3H); LC/MS: M−H=452.19.

### Step 5: Preparation of tert-butyl (5-((2-amino-6-(1-(difluoromethyl)-1H-imidazol-2-yl)phenyl)amino)hexyl)carbamate

Pd/C (434 mg, 4.08 mmol) was added to a solution of tert-butyl (5-((2-(1-(difluoromethyl)-1H-imidazol-2-yl)-6-nitrophenyl)amino)hexyl)carbamate (1.1 g, 2.04 mmol) in MeOH (56 ml). The reaction was hydrogenated under a hydrogen atmosphere at rt for 3 h. The reaction mixture was filtered through celite and the filtrate was concentrated under reduced pressure. The crude product was used without further purification.

¹H NMR (DMSO-d₆) δ: 8.03 (m, 2H), 7.34 (m, 2H), 6.93 (m, 1H), 3.89 (m, 2H), 3.16 (m, 1H), 1.06 (m, 2H); LC/MS: M+H=424.51.

### Step 6: Preparation of tert-butyl (5-(2-amino-7-(1-(difluoromethyl)-1H-imidazol-2-yl)-1H-benzo[d]imidazol-1-yl)hexyl)carbamate

Cyanogen bromide (55 mg, 519 μmol) was added to a solution of tert-butyl (5-((2-amino-6-(1-(difluoromethyl)-1H-imidazol-2-yl)phenyl)amino)hexyl)carbamate (0.9 g, 2.13 mmol) in MeOH (5 ml), MeCN (2.0 ml) and water (2.0 ml). The reaction mixture was stirred at 55° C. for 3 h. The reaction mixture was diluted with water, extracted with EtOAc and concentrated under reduced pressure. The crude residue was purified by flash chromatography (SiO2, 0 to 10% MeOH) to give tert-butyl (5-(2-amino-7-(1-(difluoromethyl)-1H-imidazol-2-yl)-1H-benzo[d]imidazol-1-yl)hexyl)carbamate (900 mg, 89%).

### Step 7: Preparation of tert-butyl 3-((1-(6-((tert-butoxycarbonyl)amino)hexan-2-yl)-7-(1-(difluoromethyl)-1H-imidazol-2-yl)-1H-benzo[d]imidazol-2-yl)carbamoyl)benzoate

To a solution of tert-butyl (5-(2-amino-7-(1-(difluoromethyl)-1H-imidazol-2-yl)-1H-benzo[d]imidazol-1-yl)hexyl)carbamate (1.1 g, 2.31 mmol) and 3-(tert-butoxycarbonyl)benzoic acid (769 mg, 3.46 mmol) in DMF (5.0 ml) and MeCN (2.0 ml) was added TCFH (782 mg, 5.76 mmol) followed by 1-methyl-1H-imidazole (946 mg, 2.31 mmol). The reaction was stirred at rt for 1 h. After completion, the reaction mixture was quenched with water and extracted with EtOAc and the organic phase concentrated under reduced pressure. The product was purified with flash chromatography (SiO₂, 45% EtOAc\Heptane) to give tert-butyl 3-((1-(6-((tert-butoxycarbonyl)amino)hexan-2-yl)-7-(1-(difluoromethyl)-1H-imidazol-2-yl)-1H-benzo[d]imidazol-2-yl)carbamoyl)benzoate (900 mg, 50%).

LC/MS: M+H=654.01.

### Step 8: Preparation of 3-((1-(6-aminohexan-2-yl)-7-(1-(difluoromethyl)-1H-imidazol-2-yl)-1H-benzo[d]imidazol-2-yl)carbamoyl)benzoic acid

TFA (2.0 ml) was added to a solution of tert-butyl 3-((1-(6-((tert-butoxycarbonyl)amino)hexan-2-yl)-7-(1-(difluoromethyl)-1H-imidazol-2-yl)-1H-benzo[d]imidazol-2-yl)carbamoyl)benzoate (0.9 g, 1.17 mmol) in CH₂Cl₂ (7.65 ml) at 0° C. The reaction was stirred at rt for 16 h. The crude reaction mixture concentrated under reduced pressure and the product was used without further purification.

LC/MS: M+H=497.57.

### Step 9: Preparation of (E)-11-methyl-1⁷-(1-methyl-1H-imidazol-2-yl)-1²,1³-dihydro-1¹H-2,6-diaza-1(2,1)-benzo[d]imidazola-4(1,3)-benzenacycloundecaphane-3,5-dione

To a solution of 3-((1-(6-aminohexan-2-yl)-7-(1-(difluoromethyl)-1H-imidazol-2-yl)-1H-benzo[d]imidazol-2-yl)carbamoyl)benzoic acid (150 mg, 302 μmol) in MeCN (350 ml) and DMF (72 ml) was added TCFH (102 mg, 744 μmol) and 1-methyl-1H-imidazole (124 mg, 1.41 mmol) at 0° C. The reaction mixture was stirred at rt for 2 h. The reaction mixture was concentrated under reduced pressure and the residue was triturated with water, filtered and dried. The crude was purified by preparative HPLC to afford racemic (E)-1⁷-(1-(difluoromethyl)-1H-imidazol-2-yl)-11-methyl-1²,1³-dihydro-1¹H-2,6-diaza-1(2,1)-benzo[d]imidazola-4(1,3)-benzenacycloundecaphane-3,5-dione (42 mg). The enantiomers were separated using chiral chromatography.

### Example 90, Enantiomer 1: (12 mg)

¹H NMR (DMSO-d₆) δ: 8.82 (s, 1H), 8.10 (d, 1H), 7.78 (d, 1H), 7.75 (m, 2H), 7.57 (m, 1H), 7.47 (m, 1H), 7.30 (m, 2H), 7.14 (m, 1H), 3.48 (m, 1H), 1.86 (m, 1H), 1.38 (m, 3H); LC/MS: M+H=479.41.

Chiralpak-IG (4.6×250) 5 um, Mobile Phase: IPA/MeOH 50:50 (v/v) Flow Rate: 1 ml/min), Rt=7.04 min.

### Example 91, Enantiomer 2: (10 mg)

¹H NMR (DMSO-d₆) δ: 8.82 (s, 1H), 8.10 (d, 1H), 7.78 (d, 1H), 7.75 (m, 2H), 7.57 (m, 1H), 7.47 (m, 1H), 7.30 (m, 2H), 7.14 (m, 1H), 3.48 (m, 1H), 1.86 (m, 1H), 1.38 (m, 3H); LC/MS: M+H=479.31.

Chiralpak-IG (4.6×250) 5 μm, Mobile Phase: IPA/MeOH 50:50 (v/v) Flow Rate: 1 ml/min, Rt=8.69 min.

### Examples 92 and 93: (E)-1⁷-((R)-3-fluoropyrrolidine-1-carbonyl)-11-methyl-1²,1³-dihydro-1¹H-2,6-diaza-1(2,1)-benzo[d]imidazola-4(1,3)-benzenacycloundecaphane-3,5-dione

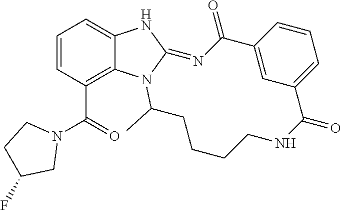 [SMILES: CC1CCCCNC(=O)c2cccc(c2)C(=O)/N=C2\Nc3cccc(C(=O)N4CC[C@@H](F)C4)c3N21]

Prepared according to general method B starting from (E)-11-methyl-3,5-dioxo-1²,1³-dihydro-1¹H-2,6-diaza-1(2,1)-benzo[d]imidazola-4(1,3)-benzenacycloundecaphane-1⁷-carboxylic acid and (3R)-3-fluoropyrrolidine hydrochloride. The diastereomers were separated using chiral chromatography.

### Example 92, Diastereomer 1

¹H NMR (DMSO-d₆) δ: 8.88 (s, 1H), 8.10 (d, 1H), 7.75 (d, 1H), 7.59 (m, 1H), 7.50 (m, 1H), 7.25 (m, 2H), 7.12 (m, 1H), 5.36 (m, 1H), 4.80 (m, 1H), 1.60 (d, 3H);

LC/MS: M+H=478.28.

Chiralpak-IG (4.6×250) 5 um, Mobile Phase: 3% EtOH in CH₂Cl₂ (v/v) Flow Rate: 1 ml/min), Rt=20.94 min.

### Example 93, Diastereomer 2

¹H NMR (DMSO-d₆) @ 253.7K δ: 8.88 (s, 1H), 8.10 (d, 1H), 7.75 (d, 1H), 7.59 (m, 1H), 7.50 (m, 1H), 7.25 (m, 2H), 7.11 (m, 1H), 5.35 (m, 1H), 4.79 (m, 1H), 1.60 (d, 3H); LC/MS: M+H=478.28.

Chiralpak-IG (4.6×250) 5 μm, Mobile Phase: 3% EtOH in CH₂Cl₂ (v/v) Flow Rate: 1 ml/min), Rt=24.88 min.

### Example 94: (R,E)-1⁷-(3-hydroxy-3-methylazetidine-1-carbonyl)-11-methyl-1²,1³-dihydro-1¹H-2,6-diaza-1(2,1)-benzo[d]imidazola-4(1,3)-benzenacycloundecaphane-3,5-dione

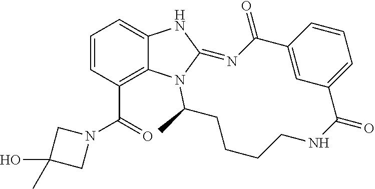 [SMILES: C[C@@H]1CCCCNC(=O)c2cccc(c2)C(=O)/N=C2\Nc3cccc(C(=O)N4CC(C)(O)C4)c3N21]

Prepared according to general method B starting from (R,E)-11-methyl-3,5-dioxo-1²,1³-dihydro-1¹H-2,6-diaza-1(2,1)-benzo[d]imidazola-4(1,3)-benzenacycloundecaphane-1⁷-carboxylic acid and 3-methylazetidin-3-ol.

¹H NMR (DMSO-d₆) δ: 8.83 (1H. s), 8.10 (1H, d), 7.81 (1H), m), 7.75 (1H, d), 7.59 (2H, m), 7.23 (1H, m), 7.13 (1H, m), 5.75 (1H, d), 3.95 (2H, m), 3.87 (1H, m)

LC/MS: M+H=476.35.

### Example 95: (S,E)-1⁷-(3-hydroxy-3-methylazetidine-1-carbonyl)-11-methyl-1²,1³-dihydro-1¹H-2,6-diaza-1(2,1)-benzo[d]imidazola-4(1,3)-benzenacycloundecaphane-3,5-dione

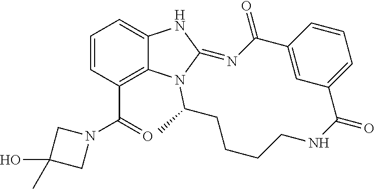 [SMILES: C[C@H]1CCCCNC(=O)c2cccc(c2)C(=O)/N=C2\Nc3cccc(C(=O)N4CC(C)(O)C4)c3N21]

Prepared according to general method B starting from (S,E)-11-methyl-3,5-dioxo-1²,1³-dihydro-1¹H-2,6-diaza-1(2,1)-benzo[d]imidazola-4(1,3)-benzenacycloundecaphane-1⁷-carboxylic acid and 3-methylazetidin-3-ol.

¹H NMR (DMSO-d₆) δ: 8.83 (1H. s), 8.10 (1H, d), 7.81 (1H), m), 7.75 (1H, d), 7.59 (2H, m), 7.23 (1H, m), 7.13 (1H, m), 5.75 (1H, d), 3.95 (2H, m), 3.87 (1H, m)

LC/MS: M+H=476.41.

### Example 96: (S,E)-1⁷-(4,5-dimethyl-4H-1,2,4-triazol-3-yl)-11-methyl-1²,1³-dihydro-1¹H-2,6-diaza-1(2,1)-benzo[d]imidazola-4(1,3)-benzenacycloundecaphane-3,5-dione

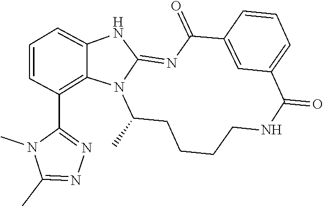 [SMILES: Cc1nnc(-c2cccc3c2N2/C(=N/C(=O)c4cccc(c4)C(=O)NCCCC[C@@H]2C)N3)n1C]

Example 96 was prepared in the same manner as Example 57: (R,E)-1⁷-(4,5-dimethyl-4H-1,2,4-triazol-3-yl)-11-methyl-1²,1³-dihydro-1¹H-2,6-diaza-1(2,1)-benzo[d]imidazola-4(1,3)-benzenacycloundecaphane-3,5-dione but using tert-butyl (S)-(5-aminohexyl)carbamate instead of tert-butyl (R)-(5-aminohexyl)carbamate.

### Example 97: (E)-1⁶-(4-methylpiperazine-1-carbonyl)-1²,1³-dihydro-1¹H-2,6-diaza-1(2,1)-benzo[d]imidazola-4(1,3)-benzenacycloundecaphane-3,5-dione

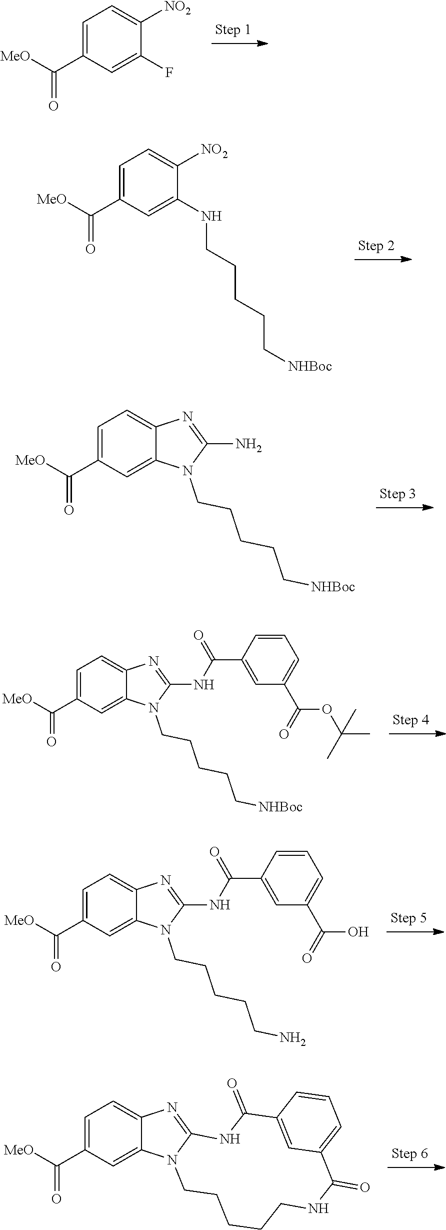 [SMILES: COC(=O)c1ccc([N+](=O)[O-])c(F)c1 ; COC(=O)c1ccc([N+](=O)[O-])c(NCCCCCNC(=O)OC(C)(C)C)c1 ; COC(=O)c1ccc2nc(N)n(CCCCCNC(=O)OC(C)(C)C)c2c1 ; COC(=O)c1ccc2nc(NC(=O)c3cccc(C(=O)OC(C)(C)C)c3)n(CCCCCNC(=O)OC(C)(C)C)c2c1 ; COC(=O)c1ccc2nc(NC(=O)c3cccc(C(=O)O)c3)n(CCCCCN)c2c1 ; COC(=O)c1ccc2nc3n(c2c1)CCCCCNC(=O)c1cccc(c1)C(=O)N3]

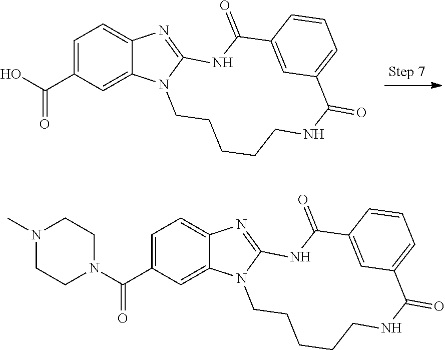 [SMILES: O=C(O)c1ccc2nc3n(c2c1)CCCCCNC(=O)c1cccc(c1)C(=O)N3 ; CN1CCN(C(=O)c2ccc3nc4n(c3c2)CCCCCNC(=O)c2cccc(c2)C(=O)N4)CC1]

### Step 1: Preparation of methyl 3-((5-((tert-butoxycarbonyl)amino)pentyl)amino)-4-nitrobenzoate

N-Boc-1,5-diaminopentane (1.21 g, 6.0 mmol) was added to a solution of methyl 3-fluoro-4-nitrobenzoate (996 mg, 5.0 mmol) in MeCN (25 ml) giving an intense yellow solution. K₂CO₃ (2.07 g, 15.0 mmol) was added giving a suspension and the mixture was heated to reflux (80° C.). The reaction mixture was concentrated to dryness and the residue partitioned between water and ethyl acetate. The organic phase was washed with 0.1 M NaOH, 0.1 M HCl, sat. NaHCO₃ and brine, dried using a phase separator and concentrated to solids (2.56 g). The product was used without further purification.

LC/MS: M+H-Boc=281.1, Rt=5.28.

### Step 2: Preparation of methyl 2-amino-1-(5-((tert-butoxycarbonyl)amino)pentyl)-1H-benzo[d]imidazole-6-carboxylate

To a solution of methyl 3-((5-((tert-butoxycarbonyl)amino)pentyl)amino)-4-nitrobenzoate (1.91 g, 5.0 mmol) in MeOH (35 ml) under N₂, was added 10% Pd/C (266 mg, 250 μmol) and the mixture was hydrogenated at rt. The mixture was evacuated, filtered through celite with MeOH (40 mL). The yellow brown solution was concentrated to a residue (2.085 g). The residue was dissolved in MeOH (15 ml) and water (3 ml) and CNBr (636 mg, 6.0 mmol) was added to the solution at rt. The reaction mixture was added to aq. Na₂CO₃ (50 mL, sat.) and the mixture was extracted with EtOAc (3×30 mL). The organic layer was dried over Mg2SO4, filtered and concentrated to yellow solids (2.2 g). The residues were purified by flash chromatography on silica gel (Biotage 12 g) using 1-7% MeOH in CH₂Cl₂ with 1% NH3 (28% aq.) to give methyl 2-amino-1-(5-((tert-butoxycarbonyl)amino)pentyl)-1H-benzo[d]imidazole-6-carboxylate as a yellow solid (1.605 g, 85%).

LC/MS: M+H=377.2, Rt=4.15.

### Step 3: Preparation of methyl 1-(5-((tert-butoxycarbonyl)amino)pentyl)-2-(3-(tert-butoxycarbonyl)benzamido)-1H-benzo[d]imidazole-6-carboxylate

HATU (882 mg, 2.32 mmol) was added to a solution of methyl 2-amino-1-(5-((tert-butoxycarbonyl)amino)pentyl)-1H-benzo[d]imidazole-6-carboxylate (728 mg, 1.93 mmol) and 3-(tert-butoxycarbonyl)benzoic acid (473 mg, 2.13 mmol) in MeCN (50 ml) at rt, followed by the addition of N,N-diisopropylethylamine (1.0 ml, 5.80 mmol) to give a yellow solution. The reaction mixture was stirred overnight and then concentrated. To the residue was added aq. NaOH (1 M) and brine followed by extraction with EtOAc (3×). The organic phases were combined and dried and concentrated to solids (1.38 g). The crude product was purified by flash chromatography on silica gel (Biotage 12 g) using 10-50% EtOAc in pet. ether to give methyl 1-(5-((tert-butoxycarbonyl)amino)pentyl)-2-(3-(tert-butoxycarbonyl)benzamido)-1H-benzo[d]imidazole-6-carboxylate (705 mg, 62%).

LC/MS: M+H=581.3, Rt=5.80.

### Step 4: Preparation of 3-((1-(5-aminopentyl)-6-(methoxycarbonyl)-1H-benzo[d]imidazol-2-yl)carbamoyl)benzoic acid

HCl (10 ml) was added to a solution of methyl 1-(5-((tert-butoxycarbonyl)amino)pentyl)-2-(3-(tert-butoxycarbonyl)benzamido)-1H-benzo[d]imidazole-6-carboxylate (200 mg, 344 μmol) in dioxane (10 ml) at rt. The reaction mixture was stirred for 6 h at rt and then concentrated to dryness. The residue was heated in diethyl ether for 90 min, cooled and the solids were collected by filtration to give the desired product (170 mg) which was used in the next step without further purification.

LC/MS: M+H=462.2; Rt=3.90 min.

### Step 5: Preparation of methyl 3,5-dioxo-1¹H-2,6-diaza-1(2,1)-benzo[d]imidazola-4(1,3)-benzenacycloundecaphane-1⁶-carboxylate

A suspension of 3-((1-(5-aminopentyl)-6-(methoxycarbonyl)-1H-benzo[d]imidazol-2-yl)carbamoyl)benzoic acid (170 mg, 369 μmol) and TEA (257 μl, 1.84 mmol) in THF (123 ml) was added dropwise by an addition funnel to a solution of PyBrOP (241 mg, 516 μmol) in THF (123 ml) at rt. The reaction mixture was stirred overnight and then heated at 70° C. for 6 h. Cooled to rt and stirred overnight. The reaction mixture was filtered and the filtrate was concentrated under reduced pressure to give white solids. The white solids were resuspended in water and concentrated to dryness two times followed by resuspension from MeOH and concentration to dryness. The solids were resuspended in MeOH and concentrated to dryness. The solids were collected by filtration and washed MeOH (˜5 mL), water (˜5 mL) then MeOH (˜5 mL). The solids were dried give white solids (110 mg) which were used without further purification.

¹H NMR (DMSO-d₆) δ: 12.79 (s, 1H), 9.02 (s, 1H), 8.11 (d, 1H), 8.05 (s, 1H), 7.91-7.82 (m, 2H), 7.76 (d, 1H), 7.63-7.53 (m, 2H), 4.33 (m, 2H), 3.88 (s, 3H), 3.24-3.15 (m, 2H), 2.09-1.98 (m, 2H), 1.88-1.79 (m, 2H), 1.48-1.40 (m, 2H); LC/MS: M+H=407.1.

### Step 6: Preparation of 3,5-dioxo-1¹H-2,6-diaza-1(2,1)-benzo[d]imidazola-4(1,3)-benzenacycloundecaphane-1⁶-carboxylic acid

5 M NaOH (90 μl, 450 μmol) was added to a suspension of methyl 3,5-dioxo-1¹H-2,6-diaza-1(2,1)-benzo[d]imidazola-4(1,3)-benzenacycloundecaphane-1⁶-carboxylate (61 mg, 150 μmol) in dioxane (1.5 ml) and water (1.5 ml). The reaction mixture stirred at RT for 26 h and then 5 M HCl (90 μl) was added. The mixture was diluted with water and concentrated to dryness. The residue was resuspended in water and the product (44 mg) was collected by filtration. The product was used without further purification.

LC/MS: M+H=393.1; Rt=3.82 min.

### Step 7: Preparation of (E)-1⁶-(4-methylpiperazine-1-carbonyl)-1²,1³-dihydro-1¹H-2,6-diaza-1(2,1)-benzo[d]imidazola-4(1,3)-benzenacycloundecaphane-3,5-dione

3,5-dioxo-1¹H-2,6-diaza-1(2,1)-benzo[d]imidazola-4(1,3)-benzenacycloundecaphane-1⁶-carboxylic acid (20 mg, 51 μmol) was suspended in THF (2.0 ml) and N,N-diisopropylethylamine (53 μl, 307 μmol) was added. The reaction mixture was stirred for 2 h at RT before N-methyl piperazine (8.5 μl, 76 μmol) and HATU (29 mg, 77 μmol) were added. The reaction mixture was heated to 40° C. and stirred overnight. The reaction mixture was concentrated to dryness. The residue was dissolved in DMSO (2 mL). To the resulting solution MeCN (0.5 mL) and water (0.5 mL) and a drop of TFA were added. The solution was filtered through a syringe filter to a clear solution. The product was purified by 3 runs of reverse phase chromatography on a C18 column, prep-HPLC, using 10-30% MeCN in water containing 0.1% TFA to give (E)-1⁶-(4-methylpiperazine-1-carbonyl)-1²,1³-dihydro-1 ¹H-2,6-diaza-1(2,1)-benzo[d]imidazola-4(1,3)-benzenacycloundecaphane-3,5-dione as a white solid (9.7 mg, 32%).

¹H NMR (DMSO-d₆) δ: 12.68 (s, 1H), 9.80 (s, 1H), 9.03 (s, 1H), 8.11 (d, 1H), 7.90 (m, 1H), 7.76 (d, 1H), 7.62 (d, 1H), 7.59 (m, 1H), 7.54 (d, 1H), 7.32 (m, 1H), 4.31 (m, 2H), 3.23-3.16 (m, 3H), 3.08 (s, 2H), 2.83 (s, 3H), 2.09-1.98 (m, 2H), 1.88-1.77 (m, 2H), 1.49-1.37 (m, 2H); LC/MS: M+H=472.2.

### Example 98: (E)-1⁶-(4-(oxetan-3-yl)piperazine-1-carbonyl)-1²,1³-dihydro-1¹H-2,6-diaza-1(2,1)-benzo[d]imidazola-4(1,3)-benzenacycloundecaphane-3,5-dione

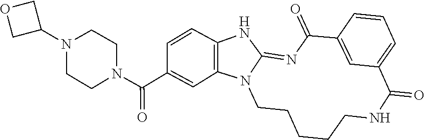 [SMILES: O=C1/N=C2\Nc3ccc(C(=O)N4CCN(C5COC5)CC4)cc3N2CCCCCNC(=O)c2cccc1c2]

3,5-dioxo-1¹H-2,6-diaza-1(2,1)-benzo[d]imidazola-4(1,3)-benzenacycloundecaphane-1⁶-carboxylic acid (18 mg, 46 μmol) was suspended in THF (3.0 ml) and N,N-diisopropylethylamine (24 μl, 140 μmol) was added. The reaction mixture was stirred for 2 h at rt before 1-(oxetan-3-yl)piperazine (10 mg, 70 μmol) and HATU (27 mg, 70 μmol) were added. The reaction mixture was heated to 40° C. and stirred overnight. The reaction mixture was concentrated to dryness. The residue was dissolved in DMSO (0.5 mL). To the resulting solution water (0.1 mL) and a drop of TFA were added. The solution was filtered through a syringe filter to a clear solution. The product was purified by 3 runs of reverse phase chromatography on a C18 column, prep-HPLC, using 10-30% MeCN in water containing 0.1% TFA to give (E)-1⁶-(4-methylpiperazine-1-carbonyl)-1²,1³-dihydro-1 ¹H-2,6-diaza-1(2,1)-benzo[d]imidazola-4(1,3)-benzenacycloundecaphane-3,5-dione as a white solid (12.4 mg, 42%).

¹H NMR (DMSO-d₆) δ: 12.67 (s, 1H), 9.03 (s, 1H), 8.11 (d, 1H), 7.89 (m, 1H), 7.76 (d, 1H), 7.62 (s, 1H), 7.59 (m, 1H), 7.52 (d, 1H), 7.30 (d, 1H), 4.83-4.52 (m, 4H), 4.30 (m, 2H), 3.26-3.13 (m, 3H), 2.10-1.99 (m, 2H), 1.88-1.78 (m, 2H), 1.49-1.34 (m, 2H); LC/MS: M+H=517.2.

### Example 99: (E)-1⁵-fluoro-1²,1³-dihydro-1¹H-2,6-diaza-1(2,1)-benzo[d]imidazola-4(1,3)-benzenacycloundecaphane-3,5-dione

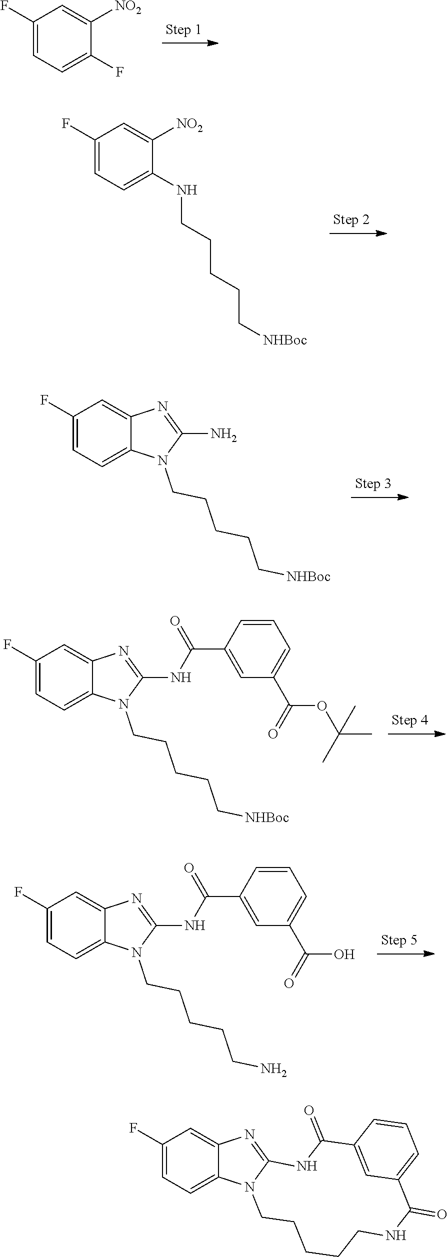 [SMILES: O=[N+]([O-])c1cc(F)ccc1F ; CC(C)(C)OC(=O)NCCCCCNc1ccc(F)cc1[N+](=O)[O-] ; CC(C)(C)OC(=O)NCCCCCn1c(N)nc2cc(F)ccc21 ; CC(C)(C)OC(=O)NCCCCCn1c(NC(=O)c2cccc(C(=O)OC(C)(C)C)c2)nc2cc(F)ccc21 ; NCCCCCn1c(NC(=O)c2cccc(C(=O)O)c2)nc2cc(F)ccc21 ; O=C1NCCCCCn2c(nc3cc(F)ccc32)NC(=O)c2cccc1c2]

### Step 1: Preparation of tert-butyl (5-((4-fluoro-2-nitrophenyl)amino)pentyl)carbamate

Potassium carbonate (261 mg, 1.89 mmol) was added to a solution of 2,5-difluoronitrobenzene (100 mg, 629 μmol) and N-boc-1,5-diaminopentane (153 mg, 754 μmol) in acetonitrile (10 ml). The resulting mixture was stirred at 50° C. overnight. The solvent was removed under reduced pressure and the residue partitioned between water and ethyl acetate. The organic phase was washed with 0.1 M NaOH, 0.1 M HCl, sat. NaHCO₃ and brine, dried over MgSO₄, filtered and concentrated. Tert-Butyl (5-((4-fluoro-2-nitrophenyl)amino)pentyl)carbamate was used in the next step without further purification.

¹H NMR (DMSO-d₆) δ: 8.06 (m, 1H), 7.84 (m, 1H), 7.51 (m, 1H), 7.11 (m, 1H), 6.76 (m, 1H), 3.37-3.30 (m, 2H), 2.90 (m, 2H), 1.60 (m, 2H), 1.40 (m, 2H), 1.35 (s, 9H), 1.34-1.28 (m, 2H); LC/MS: M+H-tert-Bu=286.0.

### Step 2: Preparation of tert-butyl (5-(2-amino-5-fluoro-1H-benzo[d]imidazol-1-yl)pentyl)carbamate

Tert-Butyl (5-((4-fluoro-2-nitrophenyl)amino)pentyl)carbamate (200 mg, 586 μmol) and 5% Pd/C (31 mg, 29 μmol) were mixed in 10 ml of ethanol. The reaction flask was evacuated and flushed with hydrogen twice. The mixture was stirred under hydrogen for 2 hours. The mixture was filtered through celite and taken directly to the next step. Cyanogen bromide (81 mg, 762 μmol) was added and the mixture was stirred overnight at 40° C. The reaction mixture was concentrated and the residue partitioned between sat. NaHCO₃ and ethyl acetate. The organic phase was separated, washed with brine, dried over MgSO₄, filtered and concentrated to give tert-butyl (5-(2-amino-5-fluoro-1H-benzo[d]imidazol-1-yl)pentyl)carbamate as a brown oil (190 mg) that was directly without further purification.

¹H NMR (DMSO-d₆) δ: 7.08 (m, 1H), 6.87 (m, 1H), 6.77 (m, 1H), 6.65 (m, 1H), 6.49 (s, 2H), 3.91 (m, 2H), 2.87 (m, 2H), 1.58 (m, 2H), 1.41-1.32 (m, 1¹H), 1.29-1.21 (m, 2H); LC/MS: M+H=337.1.

### Step 3: Preparation of tert-butyl 3-((1-(5-((tert-butoxycarbonyl)amino)pentyl)-5-fluoro-1H-benzo[d]imidazol-2-yl)carbamoyl)benzoate

A solution of tert-butyl (5-(2-amino-5-fluoro-1H-benzo[d]imidazol-1-yl)pentyl)carbamate (190 mg, 565 μmol), 3-(tert-butoxycarbonyl)benzoic acid (126 mg, 565 μmol), HATU (278 mg, 734 μmol) and TEA (236 μl, 1.69 mmol) in acetonitrile (10 ml) was stirred at 50° C. overnight. The solvent was removed under reduced pressure and the residue partitioned between water and ethyl acetate. The organic phase was washed with 0.1 M NaOH, 0.1 M HCl, sat. NaHCO₃ and brine, dried over MgSO₄, filtered and concentrated. The residue was purified with flash chromatography (silica, 10-40% EtOAc in petroleum ether) to give tert-butyl 3-((1-(5-((tert-butoxycarbonyl)amino)pentyl)-5-fluoro-1H-benzo[d]imidazol-2-yl)carbamoyl)benzoate (250 mg, 82%).

¹H NMR (DMSO-d₆) δ: 12.87 (s, 1H), 8.79 (s, 1H), 8.43 (d, 1H), 8.04 (d, 1H), 7.63-7.54 (m, 2H), 7.33 (m, 1H), 7.12 (m, 1H), 6.69 (m, 1H), 4.25 (m, 2H), 2.88 (m, 2H), 1.81 (m, 2H), 1.59 (s, 9H), 1.49-1.41 (m, 2H), 1.36-1.27 (m, 1¹H); LC/MS: M+H=541.3.

### Step 4: Preparation of 3-((1-(5-aminopentyl)-5-fluoro-1H-benzo[d]imidazol-2-yl)carbamoyl)benzoic acid

A solution of tert-butyl 3-((1-(5-((tert-butoxycarbonyl)amino)pentyl)-5-fluoro-1H-benzo[d]imidazol-2-yl)carbamoyl)benzoate (250 mg, 462 μmol) in 2 ml of dioxane, 2 ml of water and 2 ml of HCl (conc) was stirred at room temperature overnight. The reaction mixture was concentrated to dryness and the residue refluxed in diethyl ether. The solid was collected by filtration to give 3-((1-(5-aminopentyl)-5-fluoro-1H-benzo[d]imidazol-2-yl)carbamoyl)benzoic acid (160 mg) that was used without further purification.

¹H NMR (DMSO-d₆) δ: 12.87 (br. s, 1H), 8.79 (m, 1H), 8.46 (m, 1H), 8.09 (dt, J=7.7, 1.4 Hz, 1H), 7.68 (br. s, 3H), 7.62 (m, 1H), 7.58 (m, 1H), 7.35 (m, 1H), 7.14 (m, 1H), 4.28 (m, 2H), 2.75 (m, 2H), 1.84 (m, 2H), 1.61 (m, 2H), 1.39 (m, 2H); LC/MS: M+H=385.2.

### Step 5: Preparation of (E)-1⁵-fluoro-1²,1³-dihydro-1¹H-2,6-diaza-1(2,1)-benzo[d]imidazola-4(1,3)-benzenacycloundecaphane-3,5-dione

A solution of 3-((1-(5-aminopentyl)-5-fluoro-1H-benzo[d]imidazol-2-yl)carbamoyl)benzoic acid (21 mg, 50 μmol), HATU (23 mg, 60 μmol) and N,N-diisopropylethylamine (44 μl, 250 μmol) in THF (100 ml) was stirred at 50° C. overnight. The solvent was removed under reduced pressure and the residue diluted with methanol (˜50 ml), filtered and concentrated. The residue was purified with reverse phase chromatography (Gemini NX-C18, 21*150 mm, water (50 mM NH₄0H)/acetonitrile, gradient over 12 minutes, 25 ml/min). The pure fractions were pooled and concentrated to give (E)-1⁵-fluoro-1²,1³-dihydro-1¹H-2,6-diaza-1(2,1)-benzo[d]imidazola-4(1,3)-benzenacycloundecaphane-3,5-dione (3 mg, 16%).

¹H NMR (DMSO-d₆) δ: 12.58 (s, 1H), 9.02 (s, 1H), 8.10 (d, 1H), 7.88 (m, 1H), 7.74 (d, 1H), 7.58 (m, 1H), 7.53 (m, 1H), 7.27 (m, 1H), 7.14-7.07 (m, 1H), 4.27 (m, 2H), 3.23-3.14 (m, 2H), 2.07-1.98 (m, 2H), 1.85-1.77 (m, 2H), 1.45-1.38 (m, 2H);

LC/MS: M+H=367.1.

### Example 100: (E)-1⁶-(piperazine-1-carbonyl)-1²,1³-dihydro-1¹H-2,6-diaza-1(2,1)-benzo[d]imidazola-4(1,3)-benzenacycloundecaphane-3,5-dione

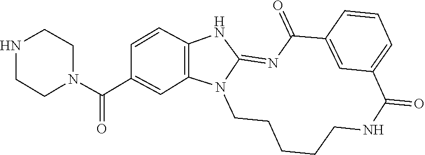 [SMILES: O=C1/N=C2\Nc3ccc(C(=O)N4CCNCC4)cc3N2CCCCCNC(=O)c2cccc1c2]

### Step 1: Preparation of tert-butyl 4-(3,5-dioxo-1¹H-2,6-diaza-1(2,1)-benzo[d]imidazola-4(1,3)-benzenacycloundecaphane-1⁶-carbonyl)piperazine-1-carboxylate

3,5-dioxo-1¹H-2,6-diaza-1(2,1)-benzo[d]imidazola-4(1,3)-benzenacycloundecaphane-1⁶-carboxylic acid (20 mg, 52 μmol) was suspended in THF (2.0 ml) and N,N-diisopropylethylamine (54 μl, 313 μmol) was added. The reaction mixture was stirred for 1 h at RT before tert-butyl piperazine-1-carboxylate (15 mg, 78 μmol) and HATU (30 mg, 78 μmol) were added. The reaction mixture was heated to 40° C. and stirred overnight. The reaction mixture was concentrated to dryness. The residue was dissolved in DMSO (0.5 mL). To the resulting solution water (0.1 mL) and a drop of TFA were added. The solution was filtered through a syringe filter to a clear solution. The product was purified by 3 runs of reverse phase chromatography on a C18 column, prep-HPLC, using 10-30% MeCN in water containing 0.1% TFA to give tert-butyl 4-(3,5-dioxo-1¹H-2,6-diaza-1(2,1)-benzo[d]imidazola-4(1,3)-benzenacycloundecaphane-1⁶-carbonyl)piperazine-1-carboxylate as a white solid (14.9 mg, 51%).

¹H NMR (DMSO-d₆) δ: 12.64 (s, 1H), 9.03 (s, 1H), 8.11 (d, 1H), 7.95-7.85 (m, 1H), 7.75 (d, 1H), 7.62-7.56 (m, 2H), 7.51 (d, 1H), 7.26 (m, 1H), 4.30 (m, 2H), 3.41-3.35 (m, 2H), 3.19 (m, 2H), 2.10-1.96 (m, 2H), 1.87-1.74 (m, 2H), 1.53-1.35 (m, 11H); LC/MS: M+H=561.3.

### Step 2: Preparation of (E)-1⁶-(piperazine-1-carbonyl)-1²,1³-dihydro-1¹H-2,6-diaza-1(2,1)-benzo[d]imidazola-4(1,3)-benzenacycloundecaphane-3,5-dione

TFA (100 μl) was added to a suspension of tert-butyl 4-(3,5-dioxo-1¹H-2,6-diaza-1(2,1)-benzo[d]imidazola-4(1,3)-benzenacycloundecaphane-1⁶-carbonyl)piperazine-1-carboxylate (10 mg, 17.8 μmol) in CH₂Cl₂ (0.5 ml) at rt giving a clear solution. The reaction was stirred for 3.5 h, concentrated to dryness under reduced pressure. The residue was dissolved in DMSO (0.5 mL), water (0.1 mL). Purified by reverse phase chromatography on a C18 column, prep-HPLC, using 10-30% MeCN in water containing 0.1% TFA over 15 min, then 5 min at highest elution strength to give (E)-1⁶-(piperazine-1-carbonyl)-1²,1³-dihydro-1¹H-2,6-diaza-1(2,1)-benzo[d]imidazola-4(1,3)-benzenacycloundecaphane-3,5-dione as a white solid (7.5 mg, 73%).

¹H NMR (DMSO-d₆) δ: 12.66 (s, 1H), 9.03 (s, 1H), 8.80 (s, 1H), 8.11 (d, 1H), 7.90 (m, 1H), 7.76 (d, 1H), 7.66-7.62 (m, 1H), 7.59 (m, 1H), 7.53 (d, 1H), 7.32 (m, 1H), 4.30 (d, 2H), 3.71 (s, 4H), 3.23-3.12 (m, 6H), 2.12-1.99 (m, 2H), 1.88-1.77 (m, 2H), 1.50-1.35 (m, 2H); LC/MS: M+H=461.2.

### Example 101: (E)-1⁶-((4-methylpiperazin-1-yl)methyl)-1²,1³-dihydro-1¹H-2,6-diaza-1(2,1)-benzo[d]imidazola-4(1,3)-benzenacycloundecaphane-3,5-dione

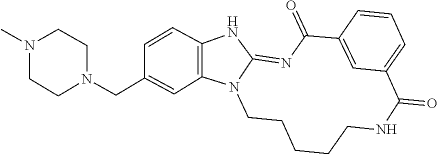 [SMILES: CN1CCN(Cc2ccc3c(c2)N2CCCCCNC(=O)c4cccc(c4)C(=O)/N=C/2N3)CC1]

### Step 1: Preparation of tert-butyl (5-(2-amino-6-(hydroxymethyl)-1H-benzo[d]imidazol-1-yl)pentyl)carbamate

LiAlH₄ (150 μL) was added dropwise to a solution of methyl 2-amino-1-(5-((tert-butoxycarbonyl)amino)pentyl)-1H-benzo[d]imidazole-6-carboxylate (94 mg, 250 μmol) in THF (5 ml) at 0° C. The reaction mixture was stirred overnight and allowed to reach rt. Water (174 μL) was added slowly followed by 15% NaOH(aq) (174 μL) and water (522 μL). The suspension was filtered with THF and the filtrate was concentrated and dried by evaporation with MeCN giving an oil (94.7 mg). The crude product was used without further purification.

LC/MS: M+H=349.2; Rt=3.80 min.

### Step 2: Preparation of tert-butyl (5-(2-amino-6-formyl-1H-benzo[d]imidazol-1-yl)pentyl)carbamate

Dess-Martin periodinane (127 mg, 300 μmol) was added to a solution of tert-butyl (5-(2-amino-6-(hydroxymethyl)-1H-benzo[d]imidazol-1-yl)pentyl)carbamate (87 mg, 250 μmol) in CH₂Cl₂ (5 ml). The reaction mixture was stirred at RT overnight and purified by reverse phase chromatography on a C18 column in four runs, prep-HPLC, using 20-50% MeCN in water containing 0.1% TFA over 20 min, then 5 min at highest elution strength to give tert-butyl (5-(2-amino-6-formyl-1H-benzo[d]imidazol-1-yl)pentyl)carbamate (40 mg, 46%) as a brown solid.

LC/MS: M+H=347.2; Rt=3.9 min.

### Step 3: Preparation of tert-butyl (5-(2-amino-6-((4-methylpiperazin-1-yl)methyl)-1H-benzo[d]imidazol-1-yl)pentyl)carbamate

TFA (100 μl) was N-methyl piperazine (58.5 mg, 585 μmol) was added to tert-butyl (5-(2-amino-6-formyl-1H-benzo[d]imidazol-1-yl)pentyl)carbamate (40 mg, 117 μmol) in THF (3 ml) followed by AcOH (7 μl, 117 μmol). Sodium triacetoxyborohydride (74 mg, 351 μmol) was added at RT. The reaction mixture was stirred overnight at RT and the diluted with EtOAc (20 ml). The solution was washed with aq. Na₂CO₃ (3×15 ml, sat) and the combined organic phases concentrated under reduced pressure. The residue was purified by reverse phase chromatography on a C18 column, prep-HPLC, using 20-40% MeCN in water containing 0.1% TFA over 20 min, then 5 min at highest elution strength to give tert-butyl (5-(2-amino-6-((4-methylpiperazin-1-yl)methyl)-1H-benzo[d]imidazol-1-yl)pentyl)carbamate (37 mg, 58%).

LC/MS: M+H=431.3; Rt=3.30 min

### Example 102: (E)-1⁵-((4-methylpiperazin-1-yl)methyl)-1²,1³-dihydro-1¹H-2,6-diaza-1(2,1)-benzo[d]imidazola-4(2,6)-pyridinacycloundecaphane-3,5-dione

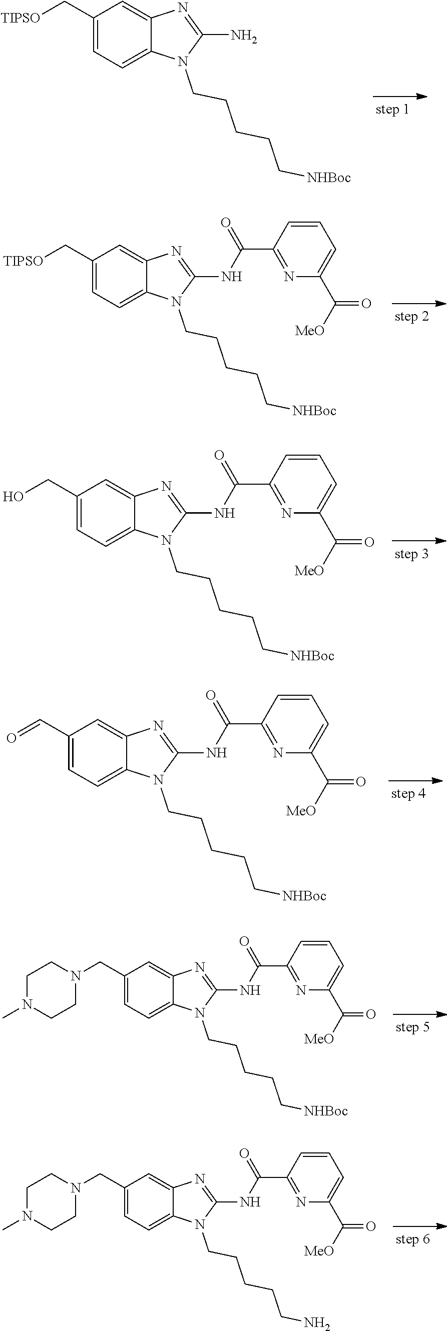

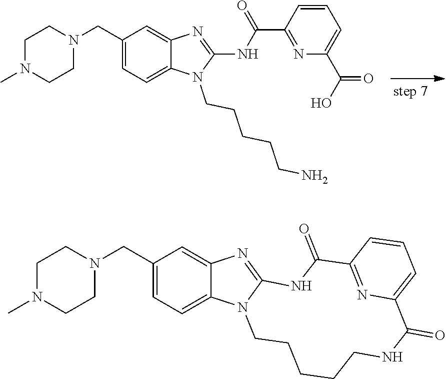 [SMILES: CN1CCN(Cc2ccc3c(c2)nc(NC(=O)c2cccc(C(=O)O)n2)n3CCCCCN)CC1 ; CN1CCN(Cc2ccc3c(c2)nc2n3CCCCCNC(=O)c3cccc(n3)C(=O)N2)CC1]

### Step 1: Preparation of methyl 6-((1-(5-((tert-butoxycarbonyl)amino)pentyl)-5-(((triisopropylsilyl)oxy)methyl)-1H-benzo[d]imidazol-2-yl)carbamoyl)picolinate

To a solution of tert-butyl N-{5-[2-amino-5-({[tris(propan-2-yl)silyl]oxy}methyl)-1H-1,3-benzodiazol-1-yl]pentyl}carbamate (1.00 g, 1.92 mmol) and 6-(methoxycarbonyl)pyridine-2-carboxylic acid (696 mg, 2 eq., 3.84 mmol) in DMF (20 ml) was added ethylbis(propan-2-yl)amine (1.02 mL, 3 eq., 5.76 mmol) at RT. After 15 min HATU ([dimethylamino(triazolo[4,5-b]pyridin-3-yloxy)methylidene]-dimethylazanium;3-hydroxytriazolo[4,5-b]pyridine;hexafluorophosphate) ((1.10 g, 1.5 eq., 2.88 mmol) was added and the mixture was stirred for 16 h at rt. The reaction mixture was diluted with cold water and extracted with EtOAc. The organic layer was concentrated under reduced pressure and the residue was dissolved in CH₂Cl₂ and purified using combi flash chromatography (SiO2, 0-10% MeOH:CH₂Cl₂) to afford methyl 6-((1-(5-((tert-butoxycarbonyl)amino)pentyl)-5-(((triisopropylsilyl)oxy)methyl)-1H-benzo[d]imidazol-2-yl)carbamoyl)picolinate (1.20 g, 1.56 mmol, 81%) as a brown oil.

¹H NMR (DMSO-d₆) δ: 8.48 (m, 1H), 8.13 (bs, 2H), 7.64 (s, 1H), 7.50 (d, 1H), 7.22 (d, 1H), 6.71 (m, 1H), 4.88 (s, 2H), 4.23 (bs, 2H), 3.93 (s, 3H), 2.87 (m, 2H), 1.79 (m, 2H), 1.45 (m, 2H), 1.31 (s, 9H), 1.18 (m, 2H), 1.08 (m, 18H); LC/MS: M+H=668.05.

### Step 2: Preparation of methyl 6-((1-(5-((tert-butoxycarbonyl)amino)pentyl)-5-(hydroxymethyl)-1H-benzo[d]imidazol-2-yl)carbamoyl)picolinate

To a solution of methyl 6-((1-(5-((tert-butoxycarbonyl)amino)pentyl)-5-(((triisopropylsilyl)oxy)methyl)-1H-benzo[d]imidazol-2-yl)carbamoyl)picolinate (1.20 g, 1.56 mmol) in THF (12.0 mL) was added 1M tetrabutylammonium fluoride (7.82 mL, 7.82 mmol) at 0° C. and stirred at rt for 1 h. The reaction mixture was concentrated under vacuum. The residue was dissolved in water and extracted with ethyl acetate to get methyl 6-((1-(5-((tert-butoxycarbonyl)amino)pentyl)-5-(hydroxymethyl)-1H-benzo[d]imidazol-2-yl)carbamoyl)picolinate (840 mg, 1.56 mmol, 99%) as brown sticky solid. The product was used in the next step without further purification.

¹H NMR (DMSO-d₆) δ: 8.48 (d, 1H), 8.11 (m, 2H), 7.56 (s, 1H), 7.48 (d, 1H), 7.21 (d, 1H), 6.73 (m, 1H), 4.56 (s, 2H), 4.24 (m, 1H), 3.92 (s, 3H), 3.126 (m, 2H), 2.87 (m, 2H), 1.79 (m, 2H), 1.56 (m, 2H), 1.46 (m, 2H), 1.31 (s, 9H); LC/MS: M+H=512.11.

### Step 3: Preparation of methyl 6-((1-(5-((tert-butoxycarbonyl)amino)pentyl)-5-formyl-1H-benzo[d]imidazol-2-yl)carbamoyl)picolinate

To a solution of methyl 6-((1-(5-((tert-butoxycarbonyl)amino)pentyl)-5-(hydroxymethyl)-1H-benzo[d]imidazol-2-yl)carbamoyl)picolinate ((840 mg, 1.56 mmol) in CH₂Cl₂ (16 ml) was added 1,1-bis(acetyloxy)-3-oxo-3H-1λ⁵,2-benziodaoxol-1-yl acetate (807 mg, 1.2 eq., 1.90 mmol) at 0° C. and stirred for 1 h at RT. The reaction mixture was filtered through Celite and concentrated under vacuum. The crude product was dissolved in ethyl acetate and washed with 1 N NaOH, dried and concentrated to get yellow oil. Crude product (650 mg) was used in next step without further purification.

¹H NMR (DMSO-d₆) δ: 9.89 (s, 1H), 8.63 (s, 1H), 8.22 (m, 2H), 8.03 (s, 1H), 7.62 (m, 1H), 7.47 (m, 1H), 4.26 (m 2H), 3.83 (s, 3H), 3.16 (m, 2H), 2.86 (m, 2H);

LC/MS: M+H=510.11.

### Step 4: Preparation of methyl 6-((1-(5-((tert-butoxycarbonyl)amino)pentyl)-5-((4-methylpiperazin-1-yl)methyl)-1H-benzo[d]imidazol-2-yl)carbamoyl)picolinate

To a solution of methyl 6-((1-(5-((tert-butoxycarbonyl)amino)pentyl)-5-formyl-1H-benzo[d]imidazol-2-yl)carbamoyl)picolinate (650 mg, 1.12 mmol) and 1-methylpiperazine (281 mg, 2.5 eq., 2.81 mmol) in CH₂Cl₂ (20 ml) was added triethylamine (778 μL, 5 eq., 5.61 mmol). The solution was stirred for 30 min at RT and then sodium triacetoxyborohydride (707 mg, 3 eq., 3.37 mmol) was added portion wise at 0° C. The reaction mixture was stirred for 16 h at rt. The reaction mixture was diluted with water and the organic layer was separated, dried over sodium sulphate, concentrated under vacuum. The crude was purified by column chromatography (silica gel, 10% MeOH/CH₂Cl₂) to afford the desired product as a colorless liquid (550 mg, 741 μmol, 66%).

¹H NMR (DMSO-d₆) δ: 8.47 (d, 1H), 8.12 (m, 2H), 7.53 (s, 1H), 7.48 (d, 1H), 7.20 (d, 1H), 4.22 (m, 2H), 4.02 (m, 2H), 3.93 (s, 3H), 2.87 (m, 2H), 1.99 (s, 3H) 1.78 (m, 2H), 1.45 (m, 2H), 1.31 (s, 9H); LC/MS: M+H=594.45.

### Step 5: Preparation of methyl 6-((1-(5-aminopentyl)-5-((4-methylpiperazin-1-yl)methyl)-1H-benzo[d]imidazol-2-yl)carbamoyl)picolinate

To a solution of methyl 6-((1-(5-((tert-butoxycarbonyl)amino)pentyl)-5-((4-methylpiperazin-1-yl)methyl)-1H-benzo[d]imidazol-2-yl)carbamoyl)picolinate (50.0 mg, 59.8 μmol) in CH₂Cl₂ (1.0 ml) was added 4M HCl in Dioxane (1.00 mL, 4.00 mmol). The reaction mixture was stirred at rt for 3 h. The reaction mixture was concentrated under vacuum and the crude compound was used in next step without any further purification.

LC/MS: M+H=494.30; Rt=1.25 min

### Step 6: Preparation of 6-((1-(5-aminopentyl)-5-((4-methylpiperazin-1-yl)methyl)-1H-benzo[d]imidazol-2-yl)carbamoyl)picolinic acid

To a solution of methyl 6-((1-(5-aminopentyl)-5-((4-methylpiperazin-1-yl)methyl)-1H-benzo[d]imidazol-2-yl)carbamoyl)picolinate (30.0 mg, 57.1 μmol) in methanol (600 μL) was added potassium hydroxide (19.2 mg, 6 eq., 343 μmol) and water (300 L). The reaction mixture was stirred at 60° C. overnight. The reaction was cooled to RT and concentrated under vacuum to get crude product. The product (40 mg) was used in the next step without further purification.

LC/MS: M−H=478.32; Rt=0.88 min

### Step 7: Preparation of (E)-1⁵-((4-methylpiperazin-1-yl)methyl)-1²,1³-dihydro-1¹H-2,6-diaza-1(2,1)-benzo[d]imidazola-4(2,6)-pyridinacycloundecaphane-3,5-dione

To a solution of 6-((1-(5-aminopentyl)-5-((4-methylpiperazin-1-yl)methyl)-1H-benzo[d]imidazol-2-yl)carbamoyl)picolinic acid (40.0 mg, 50.0 μmol) in DMF (1.0 ml) was added ethylbis(propan-2-yl)amine (44.3 μL, 5 eq., 250 μmol) at RT. The mixture was stirred 15 min before T3P (31.8 mg, 2 eq., 100 μmol) was added. Stirring was continued for 1.5 h at RT. The reaction mixture was concentrated under reduced pressure and the crude product was purified using Prep-HPLC to give the title compound (7.0 mg, 30%).

¹H NMR (DMSO-d₆) δ: 8.28 (m, 2H), 8.13 (m, 1H), 7.58 (m, 2H), 7.34 (m, 1H), 4.39 (m, 2H), 3.96 (s, 2H), 2.76 (s, 3H), 1.67 (m, 2H); LC/MS: M+H=462.28.

### Example 103: (E)-1⁵-((4-methylpiperazin-1-yl)methyl)-1²,1³-dihydro-1¹H-2,6-diaza-1(2,1)-benzo[d]imidazola-4(2,6)-pyridinacycloundecaphane-3,5-dione

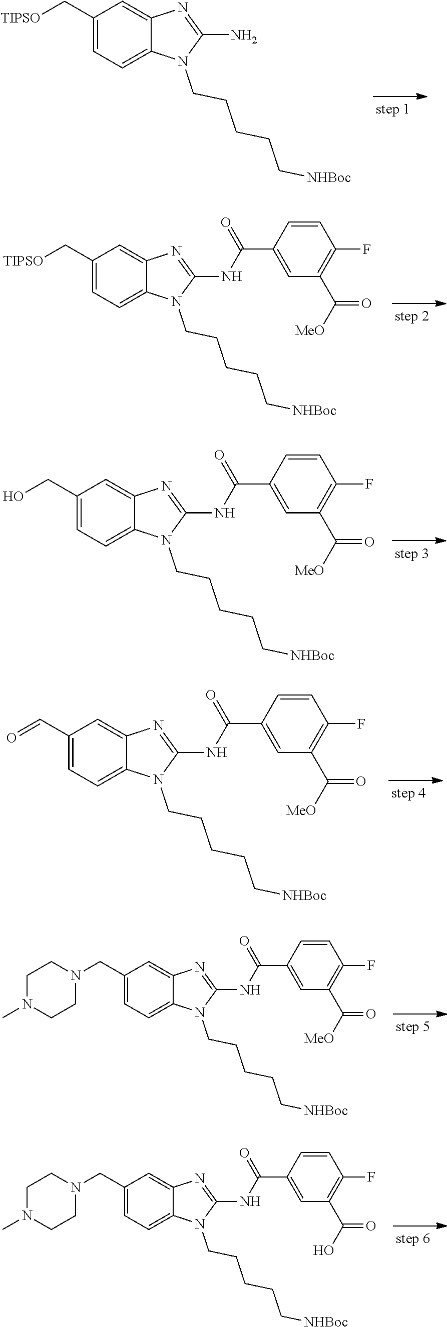

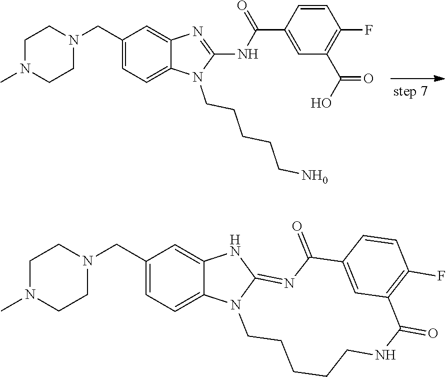

### Step 1: Preparation of methyl 5-((1-(5-((tert-butoxycarbonyl)amino)pentyl)-5-(((triisopropylsilyl)oxy)methyl)-1H-benzo[d]imidazol-2-yl)carbamoyl)-2-fluorobenzoate

To a solution of tert-butyl (5-(2-amino-5-(((triisopropylsilyl)oxy)methyl)-1H-benzo[d]imidazol-1-yl)pentyl)carbamate (1.50 g, 2.94 mmol) and 4-fluoro-3-(methoxycarbonyl)benzoic acid (1.17 g, 5.88 mmol) in DMF (15 ml) was added ethylbis(propan-2-yl)amine (1.54 mL, 8.83 mmol) at rt. After 15 min HATU (1.52 g, 6.47 mmol) was added and the mixture was stirred for 16 h at rt. The reaction mixture was diluted with cold water and extracted with EtOAc. The organic layer was concentrated under reduced pressure and purified using combi flash chromatography (SiO2, 35% EtOAc\Heptane) to afford methyl methyl 5-((1-(5-((tert-butoxycarbonyl)amino)pentyl)-5-(((triisopropylsilyl)oxy)methyl)-1H-benzo[d]imidazol-2-yl)carbamoyl)-2-fluorobenzoate (1.50 g, 73%).

¹H NMR (DMSO-d₆) δ: 8.79 (d, 1H), 8.47 (m, 1H), 7.61 (s, 1H), 7.45 (m, 2H), 7.20 (d, 1H), 6.72 (m, 1H), 4.87 (s, 2H), 4.24 (m, 2H), 3.90 (s, 3H), 2.88 (m, 2H), 1.79 (m, 2H), 1.05 (m, 21H); LC/MS: M−H=683.33.

### Step 2: Preparation of methyl 5-((1-(5-((tert-butoxycarbonyl)amino)pentyl)-5-(hydroxymethyl)-1H-benzo[d]imidazol-2-yl)carbamoyl)-2-fluorobenzoate

To a solution of methyl 5-((1-(5-((tert-butoxycarbonyl)amino)pentyl)-5-(((triisopropylsilyl)oxy)methyl)-1H-benzo[d]imidazol-2-yl)carbamoyl)-2-fluorobenzoate (1.50 g, 2.17 mmol) in THF (20.0 mL) was added 1M tetrabutylammonium fluoride (10.8 ml, 10.8 mmol) at 0° C. and stirred at rt for 1 h. The reaction mixture was concentrated under vacuum. The residue was dissolved in water and extracted with ethyl acetate to get methyl 5-((1-(5-((tert-butoxycarbonyl)amino)pentyl)-5-(hydroxymethyl)-1H-benzo[d]imidazol-2-yl)carbamoyl)-2-fluorobenzoate (1.10 g, 95%). The product was used in the next step without further purification.

¹H NMR (DMSO-d₆) δ: 8.75 (d, 1H), 8.47 (d, 1H), 7.52 (s, 1H), 7.46 (d, 1H), 7.21 (d, 1H), 6.72 (m, 1H), 4.56 (s, 2H), 4.24 (m, 1H), 3.90 (s, 3H), 2.88 (m, 2H), 1.80 (m, 2H), 0.97 (s, 9H); LC/MS: M+H=529.17.

### Step 3: Preparation of methyl 5-((1-(5-((tert-butoxycarbonyl)amino)pentyl)-5-formyl-1H-benzo[d]imidazol-2-yl)carbamoyl)-2-fluorobenzoate

To a solution of methyl 5-((1-(5-((tert-butoxycarbonyl)amino)pentyl)-5-(hydroxymethyl)-1H-benzo[d]imidazol-2-yl)carbamoyl)-2-fluorobenzoate (1.00 mg, 1.87 mmol) in CH₂Cl₂ (15 ml) was added 1,1-bis(acetyloxy)-3-oxo-3H-1λ⁵,2-benziodaoxol-1-yl acetate (953 mg, 2.25 mmol) at 0° C. and stirred for 1 h at rt. The reaction mixture was filtered through Celite and concentrated under vacuum. The crude product was dissolved in ethyl acetate and washed with 1 N NaOH, dried and concentrated to get yellow oil. The crude product (986 mg) was used in next step without further purification.

¹H NMR (DMSO-d₆) δ: 10.0 (s, 1H), 8.75 (s, 1H), 8.47 (m, 2H), 8.06 (m, 1H), 7.84 (m, 1H), 7.79 (m, 1H), 7.46 (m, 1H), 4.28 (m 2H), 3.87 (s, 3H), 2.90 (m, 2H), 1.31 (s, 9H); LC/MS: M+H=527.07.

### Step 4: Preparation of methyl 5-((1-(5-((tert-butoxycarbonyl)amino)pentyl)-5-((4-methylpiperazin-1-yl)methyl)-1H-benzo[d]imidazol-2-yl)carbamoyl)-2-fluorobenzoate

To a solution of methyl 5-((1-(5-((tert-butoxycarbonyl)amino)pentyl)-5-formyl-1H-benzo[d]imidazol-2-yl)carbamoyl)-2-fluorobenzoate (300 mg, 348 μmol) and 1-methylpiperazine (42 mg, 417 μmol) in CH₂Cl₂ (5.0 ml) was added AcOH (10 mg, 174 μmol). The solution was stirred for 30 min at RT and then sodium triacetoxyborohydride (219 mg, 1.04 mmol) was added portion wise at 0° C. The reaction mixture was stirred for 16 h at rt. The reaction mixture was diluted with water and the organic layer was separated, dried over sodium sulphate, concentrated under vacuum. The crude was purified by column chromatography (silica gel, 10% MeOH/CH₂Cl₂) to afford the desired product (150 mg, 62%).

LC/MS: M+H=611.31.

### Step 5: Preparation of 5-((1-(5-((tert-butoxycarbonyl)amino)pentyl)-5-((4-methylpiperazin-1-yl)methyl)-1H-benzo[d]imidazol-2-yl)carbamoyl)-2-fluorobenzoic acid

To a solution of methyl 5-((1-(5-((tert-butoxycarbonyl)amino)pentyl)-5-((4-methylpiperazin-1-yl)methyl)-1H-benzo[d]imidazol-2-yl)carbamoyl)-2-fluorobenzoate (200, 327 μmol) in THF (5.0 ml), water (3.0) and MeOH (2.0) was added LiOH (41 mg, 982 μmol). The reaction mixture was stirred at 50° C. for 2 h. The reaction was cooled to rt and concentrated under vacuum to get crude product. The product (200 mg) was used in the next step without further purification.

¹H NMR (DMSO-d₆) δ: 8.68 (d, 1H), 8.42 (m, 1H), 7.46 (m, 2H), 7.18 (m, 1H), 6.73 (m, 1H), 4.23 (m, 2H), 3.66 (s, 3H), 2.88 (m, 2H), 1.79 (m, 2H), 1.45 (m, 2H), 1.32 (s, 9H); LC/MS: M−H=597.11.

### Step 6: Preparation of 5-((1-(5-aminopentyl)-5-((4-methylpiperazin-1-yl)methyl)-1H-benzo[d]imidazol-2-yl)carbamoyl)-2-fluorobenzoic acid

To a solution of 5-((1-(5-((tert-butoxycarbonyl)amino)pentyl)-5-((4-methylpiperazin-1-yl)methyl)-1H-benzo[d]imidazol-2-yl)carbamoyl)-2-fluorobenzoic acid (200 mg, 302 μmol) in CH₂Cl₂ (30 ml) was added 4 M HCl in dioxane (1.05 mL, 4.00 mmol) at 0° C. The reaction mixture was stirred at rt for 3 h. The reaction mixture was concentrated under vacuum and the crude compound was used in next step without any further purification.

¹H NMR (DMSO-d₆) δ: 8.74 (s, 1H), 8.47 (m, 1H), 7.73 (m, 1H), 7.66 (m, 1H), 7.58 (m, 1H), 7.43 (m, 1H), 4.45 (m 2H), 4.29 (m, 2H), 2.88 (m, 2H), 1.84 (m, 2H), 1.64 (m, 2H); LC/MS: M+H=497.06.

### Step 7: Preparation of (E)-4⁴-fluoro-1⁵-((4-methylpiperazin-1-yl)methyl)-1²,1³-dihydro-1¹H-2,6-diaza-1(2,1)-benzo[d]imidazola-4(1,3)-benzenacycloundecaphane-3,5-dione

To a solution of 5-((1-(5-aminopentyl)-5-((4-methylpiperazin-1-yl)methyl)-1H-benzo[d]imidazol-2-yl)carbamoyl)-2-fluorobenzoic acid (200 mg, 251 μmol) in DMF (15 ml) and MeCN (100 ml) was added TCFH ((176 mg, 628 μmol) and 1-methyl-1H-imidazole (206 mg, 2.51 mmol) at 0° C. Stirring was continued for 2 h at rt. The reaction mixture was concentrated under reduced pressure and the crude product was purified using Prep-HPLC to give the title compound (20 mg, 16%).

¹H NMR (DMSO-d₆) δ: 8.89 (d, 1H), 8.07 (m, 2H), 7.50 (d, 1H), 7.45 (s, 1H), 7.36 (m, 1H), 7.24 (d, 1H), 4.28 (m, 2H), 3.39 (m, 2H), 2.77 (s, 3H), 1.97 (m, 2H), 1.77 (m, 2H, 1.37 (m, 2H); LC/MS: M+H=479.3.

### Example 104: (1²E,4²Z)-1⁵-((4-methylpiperazin-1-yl)methyl)-1²,1³-dihydro-1¹H-2,6-diaza-1(2,1)-benzo[d]imidazola-4(4,2)-thiophenacycloundecaphane-3,5-dione

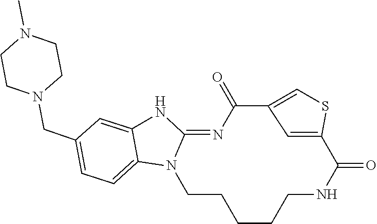 [SMILES: CN1CCN(Cc2ccc3c(c2)N/C2=N\C(=O)c4csc(c4)C(=O)NCCCCCN23)CC1]

The title product was prepared from tert-butyl (5-(2-amino-5-(((triisopropylsilyl)oxy)methyl)-1H-benzo[d]imidazol-1-yl)pentyl)carbamate and 5-(methoxycarbonyl)thiophene-3-carboxylic acid following the same procedure as for Example 103.

¹H NMR (DMSO-d₆) δ: 8.73 (s, 1H), 8.15 (s, 1H), 7.90 (m, 1H), 7.44 (d, 1H), 7.36 (s, 1H), 7.15 (s, 1H), 4.20 (m, 2H), 3.49 (s, 2H), 2.17 (s, 3H), 1.94 (m, 2H), 1.75 (m, 2H), 1.49 (m, 2H); LC/MS: M+H=467.23.

### Example 105: (E)-4⁶-fluoro-1⁵-((4-methylpiperazin-1-yl)methyl)-1²,1³-dihydro-1¹H-2,6-diaza-1(2,1)-benzo[d]imidazola-4(1,3)-benzenacycloundecaphane-3,5-dione

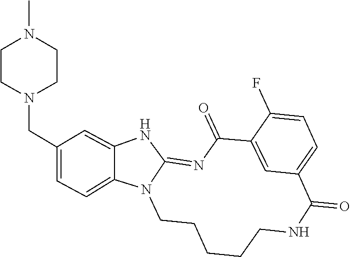 [SMILES: CN1CCN(Cc2ccc3c(c2)N/C2=N\C(=O)c4cc(ccc4F)C(=O)NCCCCCN23)CC1]

The title product was prepared from tert-butyl (5-(2-amino-5-(((triisopropylsilyl)oxy)methyl)-1H-benzo[d]imidazol-1-yl)pentyl)carbamate and 2-fluoro-5-(methoxycarbonyl)benzoic acid following the same procedure as for Example 103.

¹H NMR (DMSO-d₆) δ: 9.02 (d, 1H), 7.93 (m, 1H), 7.77 (m, 1H), 7.44 (d, 1H), 7.41 (s, 1H), 7.32 (m, 1H), 7.18 (d, 1H), 4.23 (m, 2H), 3.53 (s, 2H), 3.19 (m, 2H), 2.26 (s, 3H), 2.00 (m, 2H), 1.82 (m, 2H), 1.42 (m, 2H); LC/MS: M+H=479.36.

### Example 106: (S,E)-1⁷-((R)-3-hydroxy-3-methylpyrrolidine-1-carbonyl)-11-methyl-1²,1³-dihydro-1¹H-2,6-diaza-1(2,1)-benzo[d]imidazola-4(1,3)-benzenacycloundecaphane-3,5-dione

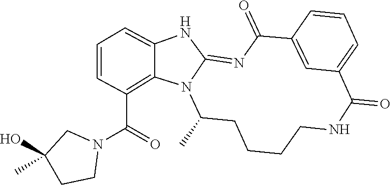 [SMILES: C[C@H]1CCCCNC(=O)c2cccc(c2)C(=O)/N=C2\Nc3cccc(C(=O)N4CC[C@@](C)(O)C4)c3N21]

Prepared according to general method B starting from (S,E)-11-methyl-3,5-dioxo-1²,1³-dihydro-1¹H-2,6-diaza-1(2,1)-benzo[d]imidazola-4(1,3)-benzenacycloundecaphane-1⁷-carboxylic acid and (R)-3-methylpyrrolidin-3-ol using standard amide coupling conditions.

¹H NMR (DMSO-d₆) @ 353 K δ: 8.88 (s, 1H), 8.10 (d, 1H), 7.75 (d, 1H), 7.57 (m, 2H), 7.48 (m, 1H), 7.22 (m, 1H), 7.08 (m, 1H), 4.84 (m, 1H), 3.66 (m, 1H), 1-94-1.77 (m, 6H), 1.62 (d, 3H), 1.45 (m, 1H); LC/MS: M+H=490.17.

### Example 107: (S,E)-1⁷-(3-cyclopropoxyazetidine-1-carbonyl)-11-methyl-1²,1³-dihydro-1¹H-2,6-diaza-1(2,1)-benzo[d]imidazola-4(1,3)-benzenacycloundecaphane-3,5-dione

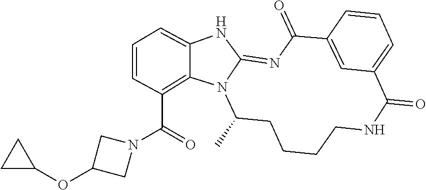 [SMILES: C[C@H]1CCCCNC(=O)c2cccc(c2)C(=O)/N=C2\Nc3cccc(C(=O)N4CC(OC5CC5)C4)c3N21]

Prepared according to general method B starting from (S,E)-11-methyl-3,5-dioxo-1²,1³-dihydro-1¹H-2,6-diaza-1(2,1)-benzo[d]imidazola-4(1,3)-benzenacycloundecaphane-1⁷-carboxylic acid and 3-cyclopropoxyazetidine using standard amide coupling conditions.

¹H NMR (DMSO-d₆) δ: 8.84 (s, 1H), 8.10 (d, 1H), 7.81 (m, 1H), 7.75 (d, 1H), 7.59 (m, 2H), 7.24 (m, 1H), 7.15 (d, 1H), 4.43 (m, 1H), 4.31 (m, 1H), 4.15 (m, 1H), 1.62 (d, 3H), 0.56 (m, 2H), 0.45 (m, 2H); LC/MS: M+H=502.28.

### Examples 108 and 109: (1²Z,4²E)-N,N,5-trimethyl-2,11-dioxo-4²,4³-dihydro-4¹H-3,10-diaza-1(4,2)-thiazola-4(2,1)-benzo[d]imidazolacycloundecaphane-4⁷-carboxamide

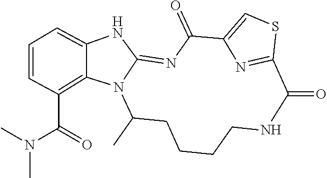 [SMILES: CC1CCCCNC(=O)c2nc(cs2)C(=O)/N=C2\Nc3cccc(C(=O)N(C)C)c3N21]

### Step 1: Preparation of ethyl 4-((1-(6-((tert-butoxycarbonyl)amino)hexan-2-yl)-7-(dimethylcarbamoyl)-1H-benzo[d]imidazol-2-yl)carbamoyl)thiazole-2-carboxylate

To a solution of tert-butyl (5-(2-amino-7-(dimethylcarbamoyl)-1H-benzo[d]imidazol-1-yl)hexyl)carbamate (1.0 g, 2.3 mmol) and 2-(ethoxycarbonyl)thiazole-4-carboxylic acid (696 mg, 3.46 mmol) in DMF (10.0 ml) was added N,N-diisopropylethylamine (1.24 ml, 6.91 mmol) and HATU (1.75 g, 4.61 mmol) at rt. The reaction mixture was stirred for 16 h. The reaction mixture was quenched with ice-cold water and extracted with EtOAc. The organic layer was washed with brine solution, dried over Na₂SO₄ and concentrated under reduced pressure. The product was used without further purification.

¹H NMR (DMSO-d₆) δ: 8.64 (m, 1H), 7.62 (m, 1H), 7.26 (m, 1H), 7.07 (m, 1H), 6.68 (m, 1H), 4.42 (m, 2H), 3.08 (m, 3H); LC/MS: M+H=587.25.

### Step 2: Preparation of 4-((1-(6-((tert-butoxycarbonyl)amino)hexan-2-yl)-7-(dimethylcarbamoyl)-1H-benzo[d]imidazol-2-yl)carbamoyl)thiazole-2-carboxylic acid

To a stirred solution of ethyl 4-((1-(6-((tert-butoxycarbonyl)amino)hexan-2-yl)-7-(dimethylcarbamoyl)-1H-benzo[d]imidazol-2-yl)carbamoyl)thiazole-2-carboxylate (1.0 g, 1.67 mmol) in THF (5.0 ml) and water (2.0 ml) was added NaOH (0.2 g, 5.01 mmol). The reaction was stirred at 60° C. for 2 h. The reaction mixture was concentrated under reduced pressure. The residue was dissolved in water and the solution was acidified with 1M HCl and extracted with CH₂Cl₂. The organic layer was concentrated under reduced pressure. The product (650 mg, 68%) was used without further purification.

¹H NMR (DMSO-d₆) δ: 8.30 (m, 1H), 7.60 (m, 1H), 7.24 (m, 1H), 7.05 (m, 1H);

LC/MS: M+H=559.25.

### Step 3: Preparation of 4-((1-(6-aminohexan-2-yl)-7-(dimethylcarbamoyl)-1H-benzo[d]imidazol-2-yl)carbamoyl)thiazole-2-carboxylic acid

HCl (146 mg, 4.01 mmol) was added to a solution of 4-((1-(6-((tert-butoxycarbonyl)amino)hexan-2-yl)-7-(dimethylcarbamoyl)-1H-benzo[d]imidazol-2-yl)carbamoyl)thiazole-2-carboxylic acid (650 mg, 803 μmol) in CH₂Cl₂ (6.9 mmol). The reaction was stirred at 0° C. for 1 h. The reaction mixture was concentrated under reduced pressure. The product (550 mg, 64%) was used without further purification.

¹H NMR (DMSO-d₆) δ: 8.30 (m, 1H), 7.60 (m, 1H), 7.24 (m, 1H), 7.05 (m, 1H);

LC/MS: M+H=459.22.

### Step 4: Preparation of (1²Z,4²E)-N,N,5-trimethyl-2,11-dioxo-4²,43-dihydro-4¹H-3,10-diaza-1(4,2)-thiazola-4(2,1)-benzo[d]imidazolacycloundecaphane-4⁷-carboxamide

TCFH (74 mg, 545 μmol) and 1-methyl-1H-imidazole (89.5 mg, 1.09 mmol) was added to a solution of 4-((1-(6-aminohexan-2-yl)-7-(dimethylcarbamoyl)-1H-benzo[d]imidazol-2-yl)carbamoyl)thiazole-2-carboxylic acid (100 mg, 218 μmol) in DMF (5.0 ml) and MeCN (200 ml). The reaction was stirred at rt for 16 h. The reaction mixture was concentrated under reduced pressure. The crude product was triturated with water and then purified by reverse phase prep HPLC to give racemic (1²Z,4²E)-N,N,5-trimethyl-2,11-dioxo-4²,4³-dihydro-4¹H-3,10-diaza-1(4,2)-thiazola-4(2,1)-benzo[d]imidazolacycloundecaphane-4⁷-carboxamide (70 mg). The enantiomers were separated using chiral chromatography.

### Example 108, Enantiomer 1

¹H NMR (DMSO-d₆) @ t=352.9 K δ: 8.33 (s, 1H), 7.95 (s, 1H), 7.41 (m, 1H), 7.12 (m, 1H), 6.92 (m, 1H), 4.51 (m, 1H), 3.56 (m, 2H), 2.84 (s, 3H), 1.75 (m, 2H), 1.55 (d, 3H); LC/MS: M+H=441.19.

Chiralpak-ID (4.6×250 mm) 5 m, Ethyl Acetate/IPA=70:30 (v/v), flow rate: 1 ml/min, Rt=6.51 min

### Example 109, Enantiomer 2

¹H NMR (DMSO-d₆) @ t=352.9 K δ: 8.37 (s, 1H), 8.00 (s, 1H), 7.46 (m, 1H), 7.19 (m, 1H), 7.01 (m, 1H), 4.52 (m, 1H), 3.56 (m, 2H), 2.85 (s, 3H), 1.82 (m, 2H), 1.55 (m, 3H); LC/MS: M+H=441.26.

Chiralpak-ID (4.6×250 mm) 5 m, Ethyl Acetate/IPA=70:30 (v/v), flow rate: 1 ml/min, Rt=7.33 min

### Example 110: (S,E)-11-methyl-1⁷-(3-(trifluoromethoxy)azetidine-1-carbonyl)-1²,1³-dihydro-1¹H-2,6-diaza-1(2,1)-benzo[d]imidazola-4(1,3)-benzenacycloundecaphane-3,5-dione

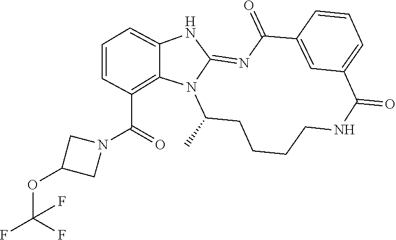 [SMILES: C[C@H]1CCCCNC(=O)c2cccc(c2)C(=O)/N=C2\Nc3cccc(C(=O)N4CC(OC(F)(F)F)C4)c3N21]

Prepared according to general method B starting from (S,E)-11-methyl-3,5-dioxo-1²,1³-dihydro-1¹H-2,6-diaza-1(2,1)-benzo[d]imidazola-4(1,3)-benzenacycloundecaphane-1⁷-carboxylic acid and 3-(trifluoromethoxy)azetidine using standard amide coupling conditions.

¹H NMR (DMSO-d₆) @ t=353 K δ: 8.90 (s, 1H), 8.10 (d, 1H), 7.75 (d, 1H), 7.62 (d, 1H), 7.58 (m, 1H), 7.52 (m, 1H), 7.24 (m, 1H), 7.17 (d, 1H), 5.26 (m, 1H), 4.95 (m, 1H), 4.42 (m, 2H), 4.15 (m, 2H), 3.46 (m, 1H), 1.95 (m, 3H), 1.64 (d, 3H), 1.51 (m, 1H); LC/MS: M+H=530.27.

### Example 111: (S,E)-1⁷-((1S,4S)-2-oxa-5-azabicyclo[2.2.1]heptane-5-carbonyl)-11-methyl-1²,1³-dihydro-1¹H-2,6-diaza-1(2,1)-benzo[d]imidazola-4(1,3)-benzenacycloundecaphane-3,5-dione

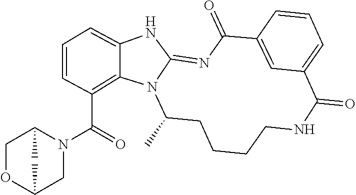 [SMILES: C[C@H]1CCCCNC(=O)c2cccc(c2)C(=O)/N=C2\Nc3cccc(C(=O)N4C[C@@H]5C[C@H]4CO5)c3N21]

Prepared according to general method B starting from (S,E)-11-methyl-3,5-dioxo-1²,1³-dihydro-1¹H-2,6-diaza-1(2,1)-benzo[d]imidazola-4(1,3)-benzenacycloundecaphane-1⁷-carboxylic acid and (1S,4S)-2-oxa-5-azabicyclo[2.2.1]heptane using standard amide coupling conditions.

¹H NMR (DMSO-d₆) @ t=353.2 K δ: 8.86 (s, 1H), 8.10 (d, 1H), 7.75 (d, 1H), 7.60 (m, 2H), 7.51 (m, 1H), 7.24 (m, 1H), 7.13 (m, 1H), 3.59 (m, 2H), 1.67 (m, 4H);

LC/MS: M+H=488.40.

### Example 112: (S,E)-1⁷-((1R,4R)-2-oxa-5-azabicyclo[2.2.1]heptane-5-carbonyl)-11-methyl-1²,1³-dihydro-1¹H-2,6-diaza-1(2,1)-benzo[d]imidazola-4(1,3)-benzenacycloundecaphane-3,5-dione

Prepared according to general method B starting from (S,E)-11-methyl-3,5-dioxo-1²,1³-dihydro-1¹H-2,6-diaza-1(2,1)-benzo[d]imidazola-4(1,3)-benzenacycloundecaphane-1⁷-carboxylic acid and (1R,4R)-2-oxa-5-azabicyclo[2.2.1]heptane using standard amide coupling conditions.

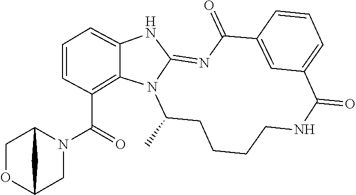 [SMILES: C[C@H]1CCCCNC(=O)c2cccc(c2)C(=O)/N=C2\Nc3cccc(C(=O)N4C[C@H]5C[C@@H]4CO5)c3N21]

¹H NMR (DMSO-d₆) @ t=353.2 K δ: 8.86 (s, 1H), 8.11 (d, 1H), 7.75 (d, 1H), 7.58 (m, 2H), 7.52 (m, 1H), 7.24 (m, 1H), 7.13 (m, 1H), 3.86 (m, 2H), 1.67 (m, 4H);

LC/MS: M+H=488.40.

### Example 113: (S,E)-11-methyl-1⁷-(2-oxopyrrolidin-1-yl)-1²,1³-dihydro-1¹H-2,6-diaza-1(2,1)-benzo[d]imidazola-4(1,3)-benzenacycloundecaphane-3,5-dione

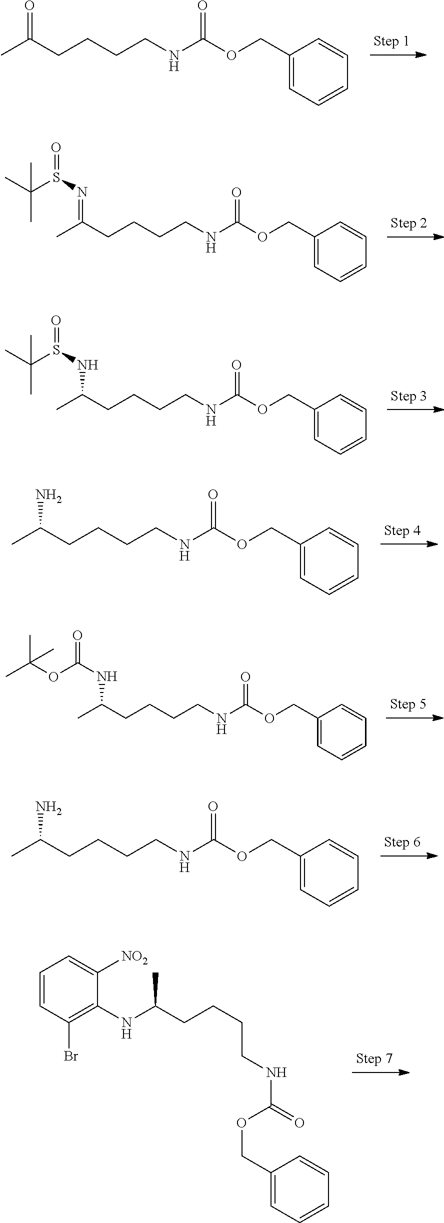

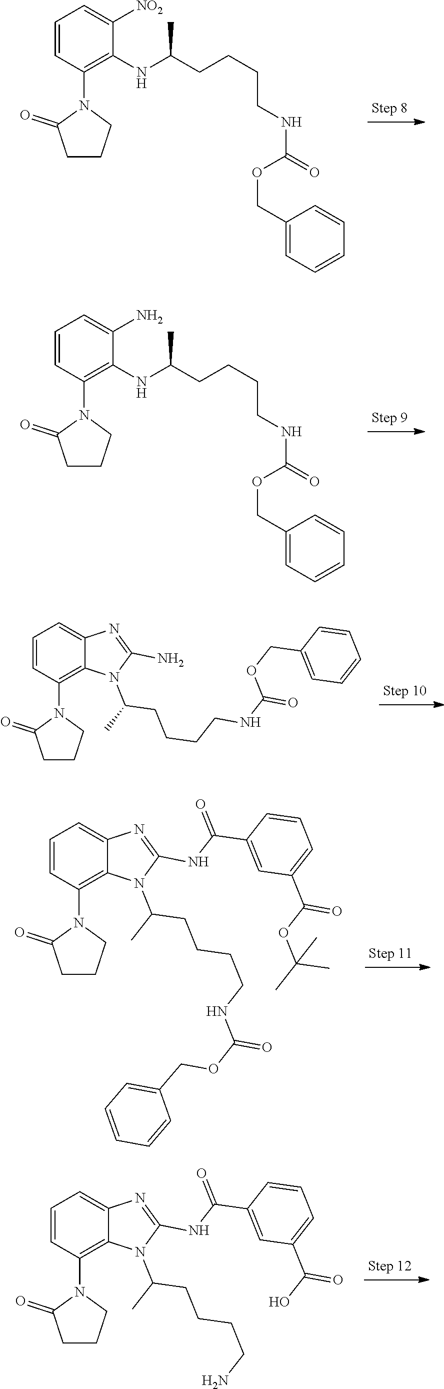

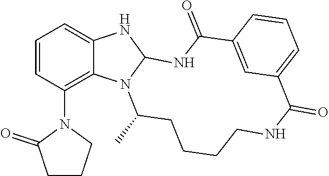 [SMILES: C[C@H]1CCCCNC(=O)c2cccc(c2)C(=O)NC2Nc3cccc(N4CCCC4=O)c3N21]

### Step 1: Preparation of benzyl (R,E)-(5-((tert-butylsulfinyl)imino)hexyl)carbamate

A solution of benzyl (5-oxohexyl)carbamate (50 g, 191 mmol), R)-2-methylpropane-2-sulfinamide (25.4 g, 210 mmol) and titanium tetraethanolate (79.9 mL, 381 mmol) was heated at 70° C. for 24 h. The reaction mixture was cooled to rt and quenched with aq NaHCO₃. The aq layer was extracted with EtOAc, dried (Na₂SO₄) and concentrated under reduced pressure. The crude residue was purified by flash chromatography (SiO₂, 0 to 80% EtOAc in heptane) to give as benzyl (R,E)-(5-((tert-butylsulfinyl)imino)hexyl)carbamate (70 g, 81%) a colorless liquid.

¹H NMR (DMSO-d₆) δ: 7.32 (m, 5H), 5.00 (m, 2H), 2.99 (m, 2H), 1.50 (m, 2H), 1.41 (m, 2H), 1.12 (s, 9H); LC/MS: M−H=351.22.

### Step 2: Preparation of benzyl ((S)-5-(((R)-tert-butylsulfinyl)amino)hexyl)carbamate

To a solution of benzyl (R,E)-(5-((tert-butylsulfinyl)imino)hexyl)carbamate (70 g, 155 mmol) in THF (500 ml) at −78° C. was added dropwise lithium tri-sec-butylborohydride, 1M solution in THF, (35.7 ml, 232 mmol). The solution was stirred at −78° C. for 3 h. The reaction mixture was quenched with water and extracted with EtOAc. The organic phase was dried (Na₂SO₄) and concentrated under reduced pressure. The product was purified by flash chromatography (SiO₂, 0 to 80% EtOAc in heptane) to give benzyl ((S)-5-(((R)-tert-butylsulfinyl)amino)hexyl)carbamate (30 g, 51%).

LC/MS: M+H=355.21.

### Step 3: Preparation of benzyl (S)-(5-aminohexyl)carbamate

A solution of benzyl ((S)-5-(((R)-tert-butylsulfinyl)amino)hexyl)carbamate (30 g, 79.5 mmol) and HCl in Dioxane (59.9 mL, 159 mmol) in CH₂Cl₂ (300 ml) was stirred at 0° C. for 4 h. The reaction mixture was concentrated under reduced pressure and the crude residue was triturated with Et₂O to give benzyl (S)-(5-aminohexyl)carbamate (21 g, 100%).

LC/MS: M+H=251.21.

### Step 4: Preparation of benzyl tert-butyl hexane-1,5-diyl(S)-dicarbamate

Triethylamine (34 ml, 242 mmol) and di-tert-butyl dicarbonate (22.2 ml, 96.6 mmol) were added to a solution of benzyl (S)-(5-aminohexyl)carbamate (21 g, 80.5 mmol) in CH₂Cl₂ (378 ml) at 0° C. The reaction mixture was stirred at rt for 8 h. The reaction mixture was quenched with water and extracted with EtOAc. The organic layer was dried (Na₂SO₄) and concentrated under reduced pressure. The crude residue was purified with flash chromatography (SiO₂, 0 to 50% EtOAc in heptane) to give benzyl tert-butyl hexane-1,5-diyl(S)-dicarbamate (11.5 g, 40%). The product was submitted to SFC (supercritical fluid chromatography) to increase stereochemical purity to 99% ee.

¹H NMR (DMSO-d₆) δ: 7.34 (m, 5H), 7.24 (m, 1H), 6.61 (m, 1H), 5.00 (s, 2H), 3.34 (m, 1H), 2.97 (m, 2H), 1.38 (s, 9H), 0.99 (d, 3H); LC/MS: M-Boc=251.18.

### Step 5: Preparation of benzyl (S)-(5-aminohexyl)carbamate

To a stirred solution of benzyl tert-butyl hexane-1,5-diyl(S)-dicarbamate (3 g, 8.22 mmol) in CH₂Cl₂ (83 ml) was added HCl in dioxane (0.5 ml, 24.7 mmol) at 0° C. The solution was stirred for 2 h at rt. The reaction mixture was concentrated under reduced pressure, triturated with Et₂O, dried under reduced pressure to give the desired product as a colourless sticky solid.

¹H NMR (DMSO-d₆) δ: 8.02 (bs, 2H), 7.30 (m, 5H), 5.00 (s, 2H), 2.95 (m, 2H), 1.59 (m, 1H), 1.41 (m, 4H), 1.10 (d, 3H).

### Step 6: Preparation of benzyl (S)-(5-((2-bromo-6-nitrophenyl)amino)hexyl)carbamate

A solution of 1-bromo-2-fluoro-3-nitrobenzene (2 g, 9.09 mmol), N,N-diisopropylethylamine (6.71 ml, 36.4 mmol) and benzyl tert-butyl hexane-1,5-diyl(S)-dicarbamate in DMF (20 ml) was stirred at 70° C. for 4 h. The reaction mixture was quenched with water and extracted with EtOAc. The organic layer was dried (Na₂SO₄) and concentrated under reduced pressure. The crude residue was purified by flash chromatography (SiO₂, 0 to 100% EtOAc in heptane) to give benzyl (S)-(5-((2-bromo-6-nitrophenyl)amino)hexyl)carbamate (2.7 g, 66%).

¹H NMR (DMSO-d₆) δ: 7.89 (m, 2H), 7.33 (m, 5H), 7.21 (m, 1H), 6.89 (m, 1H), 5.68 (d, 1H), 5.01 (s, 2H), 3.58 (m, 1H), 2.94 (m, 2H), 1.24 (m, 2H), 1.06 (d, 3H).

### Step 7: Preparation of benzyl (S)-(5-((2-nitro-6-(2-oxopyrrolidin-1-yl)phenyl)amino)hexyl)carbamate

A solution of benzyl (S)-(5-((2-bromo-6-nitrophenyl)amino)hexyl)carbamate (1.7 g, 3.77 mmol), Cs₂CO₃ (1.84 g, 5.66 mmol) and pyrrolidin-2-one (643 mg, 7.55 mmol) in 1,4-dioxane (20 ml) was purged with argon for 10 min. To the reaction mixture was added CuI (72 mg, 377 μmol) and methyl(2-(methylamino)ethyl)amine (998 mg, 11.3 mmol) and the reaction mixture was heated at 100° C. under argon for 16 h. The reaction mixture was filtered through a pad of celite, washed with EtOAc and concentrated under reduced pressure. The crude residue was purified by flash chromatography (SiO₂, 0 to 80% EtOAc in heptane) to give benzyl (S)-(5-((2-nitro-6-(2-oxopyrrolidin-1-yl)phenyl)amino)hexyl)carbamate (680 mg, 40%).

¹H NMR (DMSO-d₆) δ: 7.98 (m, 1H), 7.46 (m, 1H), 7.32 (m, 5H), 7.21 (m, 1H), 6.91 (m, 1H), 6.76 (d, 1H), 4.99 (s, 2H), 3.58 (m, 2H), 3.39 (m, 1H), 2.94 (m, 1H), 2.43 (m, 2H), 2.13 (m, 2H), 1.36 (m, 4H), 1.20 (d, 3H); LC/MS: M+H=455.20.

### Step 8: Preparation of benzyl (S)-(5-((2-amino-6-(2-oxopyrrolidin-1-yl)phenyl)amino)hexyl)carbamate

A solution of benzyl (S)-(5-((2-nitro-6-(2-oxopyrrolidin-1-yl)phenyl)amino)hexyl)carbamate (1.0 g, 2.2 mmol) and nickel (129 mg, 2.2 mmol) was stirred under a hydrogen atmosphere at rt for 2 h. The reaction mixture was filtered through a pad of celite, washed with EtOAc and concentrated under reduced pressure. The crude product was used in the next step without further purification.

¹H NMR (DMSO-d₆) δ: 7.34 (m, 5H), 7.25 (m, 1H), 6.74 (m, 1H), 6.57 (d, 1H), 6.42 (d, 1H), 5.00 (s, 2H), 3.71 (m, 2H), 3.19 (m, 1H), 2.98 (m, 2H), 2.42 (m, 2H), 2.07 (m, 2H), 1.36 (m, 2H), 1.28 (m, 2H), 0.87 (d, 3H); LC/MS: M+H=425.30.

### Step 9: Preparation of benzyl (S)-(5-(2-amino-7-(2-oxopyrrolidin-1-yl)-1H-benzo[d]imidazol-1-yl)hexyl)carbamate

A solution of benzyl (S)-(5-((2-amino-6-(2-oxopyrrolidin-1-yl)phenyl)amino)hexyl)carbamate (0.8 g, 1.88 mmol) and cyanogen bromide (220 mg, 2.07 mmol) in methanol (6.0 ml), MeCN (3.0 ml) and water (3.0 ml) was heated at 55° C. for 2 h. The reaction mixture was concentrated under reduced pressure. The residue was dissolved in NaHCO₃ (aq) and extracted with 5% MeOH in CH₂Cl₂. The organic phase was dried (Na₂SO₄) and concentrated under reduced pressure. The product was used without further purification.

¹H NMR (DMSO-d₆) δ: 7.30 (m, 5H), 7.20 (m, 1H), 7.07 (m, 1H), 6.94 (m, 1H), 6.68 (m, 1H), 4.97 (d, 2H), 3.61 (m, 1H), 2.91 (m, 2H), 2.13 (m, 1H), 1.39 (d, 3H); LC/MS: M+H=450.25.

### Step 10: Preparation of tert-butyl 3-((1-(6-(((benzyloxy)carbonyl)amino)hexan-2-yl)-7-(2-oxopyrrolidin-1-yl)-1H-benzo[d]imidazol-2-yl)carbamoyl)benzoate

TCFH (780 mg, 2.78 mmol) was added to a solution of benzyl (S)-(5-(2-amino-7-(2-oxopyrrolidin-1-yl)-1H-benzo[d]imidazol-1-yl)hexyl)carbamate (0.5 g, 1.11 mmol), 3-(tert-butoxycarbonyl)benzoic acid (494 mg, 2.22 mmol) and 1-methyl-1H-imidazole (443 μL, 5.56 mmol) in MeCN (40 ml). The reaction mixture was stirred at rt for 16 h and then quenched with ice cold water. The formed precipitate was collected by filtration and dried. The crude product was purified by flash chromatography (SiO₂, 0 to 100% EtOAc in heptane) to give tert-butyl 3-((1-(6-(((benzyloxy)carbonyl)amino)hexan-2-yl)-7-(2-oxopyrrolidin-1-yl)-1H-benzo[d]imidazol-2-yl)carbamoyl)benzoate (310 mg, 41%).

¹H NMR (DMSO-d₆) δ: 8.82 (s, 1H), 8.60 (d, 1H), 8.04 (d, 1H), 7.62 (m, 1H), 7.56 (m, 1H), 7.30 (m, 5H), 7.15 (m, 1H), 7.09 (m, 1H), 4.92 (d, 2H), 3.70 (m, 1H), 2.89 (m, 2H), 2.19 (m, 2H), 1.72 (m, 3H); LC/MS: M+H=654.70.

### Step 11: Preparation of 3-((1-(6-aminohexan-2-yl)-7-(2-oxopyrrolidin-1-yl)-1H-benzo[d]imidazol-2-yl)carbamoyl)benzoic acid

A solution of tert-butyl 3-((1-(6-(((benzyloxy)carbonyl)amino)hexan-2-yl)-7-(2-oxopyrrolidin-1-yl)-1H-benzo[d]imidazol-2-yl)carbamoyl)benzoate (0.3 g, 459 μmol) in TFA (10 ml) was heated at 60° C. for 16 h. The reaction mixture was concentrated under reduced pressure. The crude residue was triturated with Et₂O to give the product as a solid.

¹H NMR (DMSO-d₆) δ: 8.78 (s, 1H), 8.40 (d, 1H), 8.09 (d, 1H), 7.64 (m, 1H), 7.26 (m, 1H), 7.13 (m, 1H), 4.62 (d, 1H), 2.68 (m, 2H), 1.75 (m, 3H), 1.48 (m, 2H), 1.28 (m, 1H); LC/MS: M+H=464.40.

### Step 12: Preparation of (S,E)-11-methyl-1⁷-(2-oxopyrrolidin-1-yl)-1²,1³-dihydro-1¹H-2,6-diaza-1(2,1)-benzo[d]imidazola-4(1,3)-benzenacycloundecaphane-3,5-dione

A solution of 3-((1-(6-aminohexan-2-yl)-7-(2-oxopyrrolidin-1-yl)-1H-benzo[d]imidazol-2-yl)carbamoyl)benzoic acid (0.2 g, 431 μmol) in DMF (2 ml) and MeCN (100 ml) was added to a solution of TCFH (242 mg, 863 μmol) in MeCN. The resulting solution was stirred at 0° C. for 30 min before 1-methyl-1H-imidazole (172 L, 2.16 mmol) in MeCN was added at 0° C. The reaction mixture was stirred at rt for 2 h. The reaction mixture was quenched with water (5 mL) and concentrated under reduced pressure. This crude product was diluted with water. The formed precipitate was collected by filtration, washed with cold water and dried in vacuum. Crude was purified by Combiflash (3% MeOH/CH₂Cl₂) to give (S,E)-11-methyl-1⁷-(2-oxopyrrolidin-1-yl)-1²,1³-dihydro-1¹H-2,6-diaza-1(2,1)-benzo[d]imidazola-4(1,3)-benzenacycloundecaphane-3,5-dione (15 mg, 7.7%).

¹H NMR (DMSO-d₆) @ t=354.1 K δ: 8.85 (s, 1H), 8.10 (d, 1H), 7.75 (d, 1H), 7.57 (m, 1H), 7.51 (d, 1H), 7.47 (m, 1H), 7.23 (m, 1H), 7.08 (m, 1H), 4.84 (m, 1H), 3.89 (m, 1H), 3.73 (m, 1H), 3.56 (m, 1H), 2.24 (m, 2H), 2.05 (m, 1H), 1.49 (m, 1H);

LC/MS: M+H=446.30.

### Example 114: (S,E)-11-methyl-1⁷-(2-oxa-6-azaspiro[3.3]heptane-6-carbonyl)-1²,1³-dihydro-1¹H-2,6-diaza-1(2,1)-benzo[d]imidazola-4(1,3)-benzenacycloundecaphane-3,5-dione

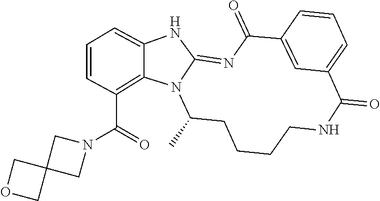 [SMILES: C[C@H]1CCCCNC(=O)c2cccc(c2)C(=O)/N=C2\Nc3cccc(C(=O)N4CC5(COC5)C4)c3N21]

Prepared according to general method B starting from (S,E)-11-methyl-3,5-dioxo-1²,1³-dihydro-1¹H-2,6-diaza-1(2,1)-benzo[d]imidazola-4(1,3)-benzenacycloundecaphane-1⁷-carboxylic acid and 2-oxa-6-azaspiro[3.3]heptane using standard amide coupling conditions.

¹H NMR (DMSO-d₆) δ: 8.83 (s, 1H), 8.10 (d, 1H), 7.82 (d, 1H), 7.75 (d, 1H), 7.59 (m, 2H), 7.52 (m, 1H), 7.23 (m, 1H), 7.14 (m, 1H), 4.68 (m, 4H), 4.28 (m, 2H), 4.16 (m, 1H), 4.08 (m, 1H), 1.90 (m, 2H); LC/MS: M+H=488.30.

### Example 115: (S,E)-11-methyl-1⁷-(2-oxa-6-azaspiro[3.4]octane-6-carbonyl)-1²,1³-dihydro-1¹H-2,6-diaza-1(2,1)-benzo[d]imidazola-4(1,3)-benzenacycloundecaphane-3,5-dione

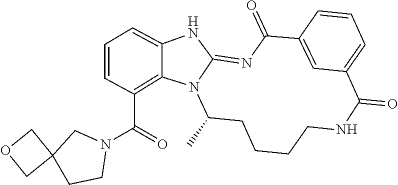 [SMILES: C[C@H]1CCCCNC(=O)c2cccc(c2)C(=O)/N=C2\Nc3cccc(C(=O)N4CCC5(COC5)C4)c3N21]

Prepared according to general method B starting from (S,E)-11-methyl-3,5-dioxo-1²,1³-dihydro-1¹H-2,6-diaza-1(2,1)-benzo[d]imidazola-4(1,3)-benzenacycloundecaphane-1⁷-carboxylic acid and 2-oxa-6-azaspiro[3.4]octane using standard amide coupling conditions.

¹H NMR (DMSO-d₆) @ t=354.2 K δ: 8.88 (s, 1H), 8.10 (d, 1H), 7.75 (d, 1H), 7.58 (m, 2H), 7.48 (m, 1H), 7.24 (m, 1H), 7.10 (m, 1H), 4.65 (m, 1H), 4.53 (m, 1H), 4.44 (m, 2H), 3.58 (m, 1H), 2.25 (m, 1H), 2.18 (m, 1H), 1.66 (d, 3H); LC/MS: M+H=502.30.

### Example 116: (S,E)-11-methyl-1⁷-(5-oxa-2-azaspiro[3.4]octane-2-carbonyl)-1²,1³-dihydro-1¹H-2,6-diaza-1(2,1)-benzo[d]imidazola-4(1,3)-benzenacycloundecaphane-3,5-dione

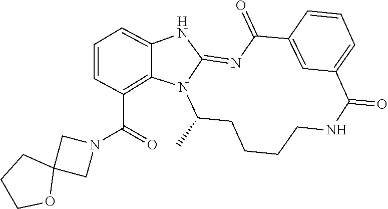 [SMILES: C[C@H]1CCCCNC(=O)c2cccc(c2)C(=O)/N=C2\Nc3cccc(C(=O)N4CC5(CCCO5)C4)c3N21]

Prepared according to general method B starting from (S,E)-11-methyl-3,5-dioxo-1²,1³-dihydro-1¹H-2,6-diaza-1(2,1)-benzo[d]imidazola-4(1,3)-benzenacycloundecaphane-1⁷-carboxylic acid and 5-oxa-2-azaspiro[3.4]octane using standard amide coupling conditions.

¹H NMR (DMSO-d₆) @ t=353.2 K δ: 8.90 (s, 1H), 8.10 (d, 1H), 7.76 (d, 1H), 7.59 (m, 2H), 7.50 (m, 1H), 7.23 (m, 1H), 7.13 (m, 1H), 4.95 (m, 1H), 4.07 (m, 1H), 3.93 (m, 2H), 3.77 (m, 2H), 3.47 (m, 1H), 1.64 (d, 3H); LC/MS: M+H=502.35.

### Example 117: (S,E)-1⁷-(7,7-difluoro-5-oxa-2-azaspiro[3.4]octane-2-carbonyl)-11-methyl-1²,1³-dihydro-1¹H-2,6-diaza-1(2,1)-benzo[d]imidazola-4(1,3)-benzenacycloundecaphane-3,5-dione

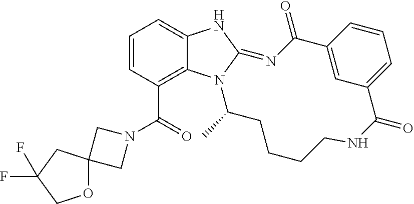 [SMILES: C[C@H]1CCCCNC(=O)c2cccc(c2)C(=O)/N=C2\Nc3cccc(C(=O)N4CC5(C4)CC(F)(F)CO5)c3N21]

Prepared according to general method B starting from (S,E)-11-methyl-3,5-dioxo-1²,1³-dihydro-1¹H-2,6-diaza-1(2,1)-benzo[d]imidazola-4(1,3)-benzenacycloundecaphane-1⁷-carboxylic acid and 7,7-difluoro-5-oxa-2-azaspiro[3.4]octane using standard amide coupling conditions.

¹H NMR (DMSO-d₆) @ t=353.2 K δ: 8.90 (s, 1H), 8.10 (d, 1H), 7.76 (d, 1H), 7.61 (m, 2H), 7.51 (m, 1H), 7.23 (m, 1H), 7.15 (m, 1H), 4.94 (m, 1H), 4.07 (m, 1H), 4.31 (m, 2H), 4.18 (m, 2H), 4.03 (m, 2H), 3.47 (m, 1H), 1.62 (d, 3H); LC/MS: M+H=538.35.

### Example 118: (S,E)-11-methyl-N,N-bis(methyl-d₃)-3,5-dioxo-1²,1³-dihydro-1¹H-2,6-diaza-1(2,1)-benzo[d]imidazola-4(1,3)-benzenacycloundecaphane-1⁷-carboxamide

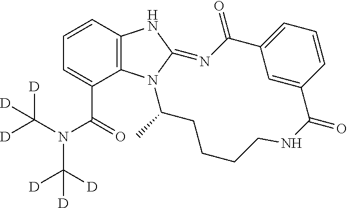 [SMILES: [2H]C([2H])([2H])N(C(=O)c1cccc2c1N1/C(=N/C(=O)c3cccc(c3)C(=O)NCCCC[C@@H]1C)N2)C([2H])([2H])[2H]]

Prepared according to general method B starting from (S,E)-11-methyl-3,5-dioxo-1²,1³-dihydro-1¹H-2,6-diaza-1(2,1)-benzo[d]imidazola-4(1,3)-benzenacycloundecaphane-1⁷-carboxylic acid and bis(methyl-d₃)amine using standard amide coupling conditions.

¹H NMR (DMSO-d₆) @ t=353.2 K δ: 8.87 (s, 1H), 8.10 (d, 1H), 7.75 (d, 1H), 7.58 (m, 2H), 7.50 (m, 1H), 7.23 (m, 1H), 7.04 (m, 1H), 3.48 (bs, 1H), 1.61 (d, 3H);

LC/MS: M+H=440.33.

### Example 119: (S,E)-11-methyl-1⁴-(2-oxopyrrolidin-1-yl)-1²,1³-dihydro-1¹H-2,6-diaza-1(2,3)-imidazo[4,5-c]pyridina-4(1,3)-benzenacycloundecaphane-3,5-dione

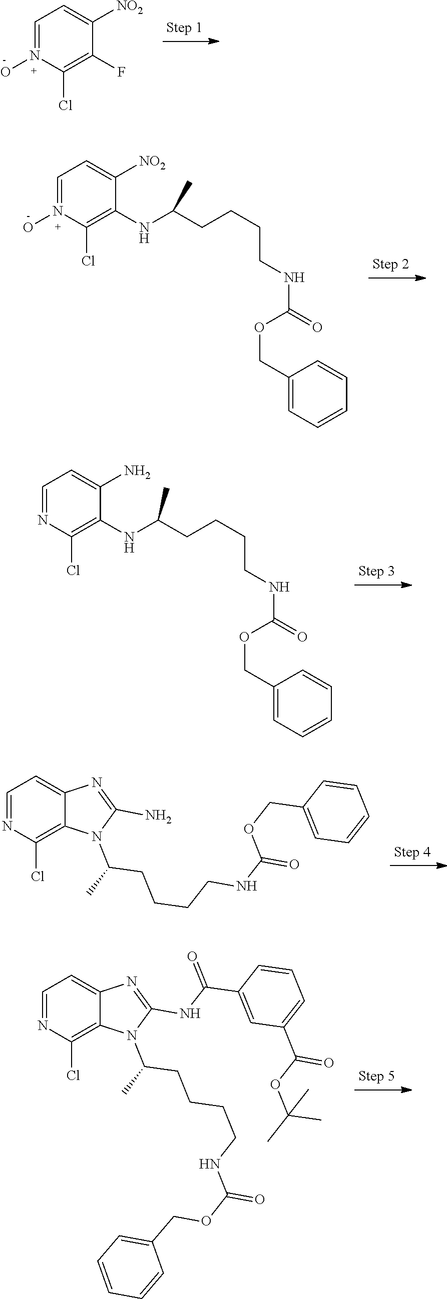

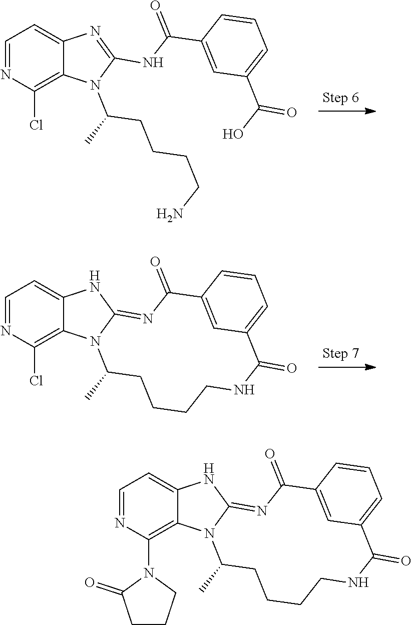 [SMILES: C[C@@H](CCCCN)n1c(NC(=O)c2cccc(C(=O)O)c2)nc2ccnc(Cl)c21 ; C[C@H]1CCCCNC(=O)c2cccc(c2)C(=O)/N=C2\Nc3ccnc(Cl)c3N21 ; C[C@H]1CCCCNC(=O)c2cccc(c2)C(=O)/N=C2\Nc3ccnc(N4CCCC4=O)c3N21]

### Step 1: Preparation (S)-3-((6-(((benzyloxy)carbonyl)amino)hexan-2-yl)amino)-2-chloro-4-nitropyridine 1-oxide

A solution of 2-chloro-3-fluoro-4-nitropyridine 1-oxide (2.8 g, 14.5 mmol), benzyl (S)-(5-aminohexyl)carbamate (5.46 g, 21.8 mmol) and N,N-diisopropylethylamine (8.06 ml, 43.6 mmol) in DMF (30 ml) was stirred at rt for 5 h. The reaction mixture was quenched with water and extracted with EtOAc. The organic phase was dried (Na₂SO₄) and concentrated under reduced pressure. The crude residue was purified by flash chromatography (SiO₂, 0 to 80% EtOAc in heptane) to give (S)-3-((6-(((benzyloxy)carbonyl)amino)hexan-2-yl)amino)-2-chloro-4-nitropyridine 1-oxide (3.5 g, 52%).

¹H NMR (DMSO-d₆) δ: 8.01 (d, 1H), 7.93 (d, 1H), 7.32 (m, 5H), 7.23 (m, 1H), 7.10 (d, 1H), 4.99 (s, 2H), 3.95 (m, 1H), 2.95 (m, 2H), 1.59 (m, 1H), 1.52 (m, 1H), 1.38 (m, 2H), 1.19 (d, 3H); LC/MS: M+H=423.00.

### Step 2: Preparation of benzyl (S)-(5-((4-amino-2-chloropyridin-3-yl)amino)hexyl)carbamate

To a stirred solution of (S)-3-((6-(((benzyloxy)carbonyl)amino)hexan-2-yl)amino)-2-chloro-4-nitropyridine 1-oxide (2 g, 4.73 mmol) in EtOAc (250 ml) was added Raney-Nickel (2 g, 34.1 mmol) at rt. The reaction mixture was stirred under a hydrogen atmosphere at rt for 2 h. The reaction mixture was filtered through a celite bed using ethyl acetate and the filtrate was concentrated under reduced pressure. The product (1.4 g) was used without further purification.

¹H NMR (DMSO-d₆) δ: 7.52 (d, 1H), 7.35 (m, 5H), 7.22 (m, 1H), 6.54 (d, 1H), 5.89 (s, 2H), 5.00 (s, 2H), 3.36 (m, 1H), 3.17 (m, 1H), 2.98 (m, 2H), 0.99 (d, 3H); LC/MS: M+H=377.00.

### Step 3: Preparation of benzyl (S)-(5-(2-amino-4-chloro-3H-imidazo[4,5-c]pyridin-3-yl)hexyl)carbamate

To a stirred solution of benzyl (S)-(5-((4-amino-2-chloropyridin-3-yl)amino)hexyl)carbamate (1.0 g, 2.65 mmol) in MeCN (10 ml), MeOH (10 ml) and water (5.0 ml). The reaction mixture was stirred at rt with cyanogen bromide (562 mg, 5.3 mmol) being added very 16 h (in total 2.81 g, 26.5 mmol). The reaction mixture was concentrated under reduced pressure and dissolved in NaHCO₃ (sat). The aqueous solution was extracted with 10% MeOH/CH₂Cl₂ (3×15 ml). The combined organic fractions were concentrated. The product was purified using flash chromatography (SiO2, 5% MeOH/CH₂Cl₂) to give benzyl (S)-(5-(2-amino-4-chloro-3H-imidazo[4,5-c]pyridin-3-yl)hexyl)carbamate (300 mg, 28%).

¹H NMR (DMSO-d₆) δ: 7.85 (m, 1H), 7.34 (m, 5H), 7.21 (m, 1H), 7.11 (d, 1H), 6.89 (m, 1H), 4.97 (s, 2H), 2.92 (m, 2H), 1.49 (m, 2H); LC/MS: M+H=401.95.

### Step 4: Preparation of tert-butyl (S)-3-((3-(6-(((benzyloxy)carbonyl)amino)hexan-2-yl)-4-chloro-3H-imidazo[4,5-c]pyridin-2-yl)carbamoyl)benzoate

To a stirred solution of 3-(tert-butoxycarbonyl)benzoic acid (387 mg, 1.74 mmol) in DMF (0.5 ml) and MeCN (20 ml) was added TCFH (733 mg, 2.61 mmol) and 1-methyl-1H-imidazole (347 μL, 4.35 mmol) at 0° C. After 15 min benzyl (S)-(5-(2-amino-4-chloro-3H-imidazo[4,5-c]pyridin-3-yl)hexyl)carbamate (350 mg, 871 μmol) was added. The resulting solution was stirred at rt for 16 h. The reaction mixture was concentrated under reduced pressure and the residue was dissolved in ice cold water. The aqueous solution extracted with ethyl acetate. The organic layer was concentrated under reduced pressure and the crude product was purified by flash chromatography (SiO₂, 50% ethyl acetate-heptane) to give tert-butyl (S)-3-((3-(6-(((benzyloxy)carbonyl)amino)hexan-2-yl)-4-chloro-3H-imidazo[4,5-c]pyridin-2-yl)carbamoyl)benzoate (250 mg, 46%).

¹H NMR (DMSO-d₆) δ: 8.38 (d, 1H), 8.17 (d, 1H), 8.07 (d, 1H), 7.65 (m, 1H), 7.56 (d, 1H), 7.32 (m, 5H), 7.16 (m, 1H), 4.92 (s, 2H); LC/MS: M+H=606.10.

### Step 5: Preparation of (S)-3-((3-(6-aminohexan-2-yl)-4-chloro-3H-imidazo[4,5-c]pyridin-2-yl)carbamoyl)benzoic acid

A solution of tert-butyl (S)-3-((3-(6-(((benzyloxy)carbonyl)amino)hexan-2-yl)-4-chloro-3H-imidazo[4,5-c]pyridin-2-yl)carbamoyl)benzoate (0.1 g, 165 μmol) in TFA (4.0 ml) was heated at 60° C. for 6 h. The reaction mixture was concentrated under reduced pressure. The crude residue was triturated with Et₂O to give the product (80 mg) as a solid.

¹H NMR (DMSO-d₆) δ: 8.79 (s, 1H), 8.41 (d, 1H), 8.19 (d, 1H), 8.12 (d, 1H), 7.65 (m, 1H), 7.59 (m, 2H), 2.72 (m, 2H), 1.55 (d, 3H); LC/MS: M+H=415.95.

### Step 6: Preparation of (S,E)-1⁴-chloro-11-methyl-1²,1³-dihydro-1¹H-2,6-diaza-1(2,3)-imidazo[4,5-c]pyridina-4(1,3)-benzenacycloundecaphane-3,5-dione

TCFH (169 mg, 431 μmol) was added to a solution of (S)-3-((3-(6-aminohexan-2-yl)-4-chloro-3H-imidazo[4,5-c]pyridin-2-yl)carbamoyl)benzoic acid (0.1 g, 240 μmol) in DMF (0.2 ml) and MeCN (400 ml). The resulting solution was stirred at 0° C. for 30 min before 1-methyl-1H-imidazole (96 μL, 1.2 mmol) was added at 0° C. The reaction mixture was stirred at rt for 3 h. The reaction mixture was quenched with water (5 mL) and concentrated under reduced pressure. This crude product was diluted with water. The formed precipitate was collected by filtration, washed with cold water and dried in vacuum. The crude was purified by Combiflash (SiO₂, 10% 1:1 ethyl acetate:MeOH in CH₂Cl₂) to give (S,E)-1⁴-chloro-11-methyl-1²,1³-dihydro-1¹H-2,6-diaza-1(2,3)-imidazo[4,5-c]pyridina-4(1,3)-benzenacycloundecaphane-3,5-dione (25 mg, 20%).

¹H NMR (DMSO-d₆) δ: 8.23 (m, 1H), 8.12 (d, 1H), 7.78 (d, 1H), 7.52 (m, 1H);

LC/MS: M+H=397.15.

### 7: Preparation of (S,E)-11-methyl-1⁴-(2-oxopyrrolidin-1-yl)-1²,1³-dihydro-1¹H-2,6-diaza-1(2,3)-imidazo[4,5-c]pyridina-4(1,3)-benzenacycloundecaphane-3,5-dione

Cs₂CO₃ (37 mg, 113 μmol) and pyrrolidin-2-one (3.21 mg, 37.7 μmol) were added to a solution of (S,E)-1⁴-chloro-11-methyl-1²,1³-dihydro-1¹H-2,6-diaza-1(2,3)-imidazo[4,5-c]pyridina-4(1,3)-benzenacycloundecaphane-3,5-dione (15 mg, 38 μmol) in dioxane (1.0 ml) at rt. The reaction mixture was purged with nitrogen for 15 minutes and then CuI (22 mg, 113 μmol) followed by 1,2-dimethylethylenediamine (13 mg, 151 μmol) were. The reaction mixture was stirred at 110° C. for 16 h. The reaction mixture was filtered through a celite bed using 10% CH₂Cl₂-MeOH. The organic layer was washed with water, dried (Na₂SO₄) and concentrated under reduced pressure. The crude product was purified by reverse phase HPLC (ammonium acetate mobile phase) to give (S,E)-11-methyl-1⁴-(2-oxopyrrolidin-1-yl)-1²,1³-dihydro-1¹H-2,6-diaza-1(2,3)-imidazo[4,5-c]pyridina-4(1,3)-benzenacycloundecaphane-3,5-dione (5 mg, 29%).

¹H NMR (DMSO-d₆+d-TFA) δ: 8.74 (bs, 1H), 8.47 (d, 1H), 8.12 (d, 1H), 7.78 (d, 1H), 7.68 (d, 1H), 7.57 (m, 1H), 4.81 (m, 1H), 3-79-3.98 (m, 2H), 3.02 (m, 1H), 1.85 (m, 3H); LC/MS: M+H=447.40.

### Examples 120 and 121: (11S,E)-11-methyl-1⁷-(1-oxa-6-azaspiro[3.4]octane-6-carbonyl)-1²,1³-dihydro-1H-2,6-diaza-1(2,1)-benzo[d]imidazola-4(1,3)-benzenacycloundecaphane-3,5-dione

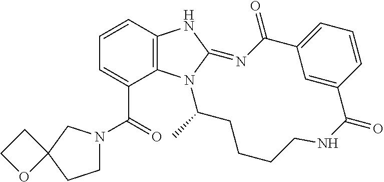 [SMILES: C[C@H]1CCCCNC(=O)c2cccc(c2)C(=O)/N=C2\Nc3cccc(C(=O)N4CCC5(CCO5)C4)c3N21]

Prepared according to general method B starting from (S,E)-11-methyl-3,5-dioxo-1²,1³-dihydro-1¹H-2,6-diaza-1(2,1)-benzo[d]imidazola-4(1,3)-benzenacycloundecaphane-1⁷-carboxylic acid and 2-oxa-6-azaspiro[3.4]octane using standard amide coupling conditions.

### Example 120, Diastereomer 1

¹H NMR (DMSO-d₆) @ 353.7 K δ: 8.71 (s, 1H), 8.10 (d, 1H), 7.75 (d, 1H), 7.58, (m, 2H), 7.48 (bs, 1H), 7.24 (m, 1H), 7.09 (m, 1H), 4.73 (bs, 1H), 4.47-4.29 (m, 2H), 1.60 (d, 3H); LC/MS: M+H=502.35.

Chiralcel OJ-H (250*4.6) mm, 5 um, CO2/(MeCN/0.2% TEA in MeOH 80:20) 80:20, Flow Rate: 4.0 mL/min, Rt=7.48 min

### Example 121, Diastereomer 2

¹H NMR (DMSO-d₆) @ 354.1 K δ: 8.88 (s, 1H), 8.10 (d, 1H), 7.75 (d, 1H), 7.58, (m, 2H), 7.47 (bs, 1H), 7.24 (m, 1H), 7.09 (m, 1H), 4.76 (bs, 1H), 4.47 (m, 1H), 4.32 (m, 1H), 4.32 (m, 1H), 3.26 (m, 1H), 1.59 (d, 3H); LC/MS: M+H=502.30.

Chiralcel OJ-H (250*4.6) mm, 5 um, CO2/(MeCN/0.2% TEA in MeOH 80:20) 80:20, Flow Rate: 4.0 mL/min, Rt=9.98 min

### Example 122: (S,E)-1⁴-(3-cyclopropyl-2-oxoimidazolidin-1-yl)-11-methyl-1²,1³-dihydro-1¹H-2,6-diaza-1(2,3)-imidazo[4,5-c]pyridina-4(1,3)-benzenacycloundecaphane-3,5-dione

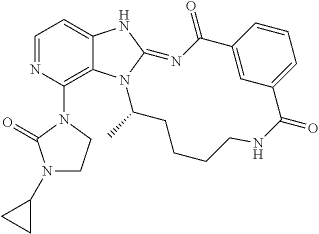 [SMILES: C[C@H]1CCCCNC(=O)c2cccc(c2)C(=O)/N=C2\Nc3ccnc(N4CCN(C5CC5)C4=O)c3N21]

Cs₂CO₃ (118 mg, 362 μmol) and 1-cyclopropylimidazolidin-2-one (22.8 mg, 163 μmol) were added to a solution of (S,E)-1⁴-chloro-11-methyl-1²,1³-dihydro-1¹H-2,6-diaza-1(2,3)-imidazo[4,5-c]pyridina-4(1,3)-benzenacycloundecaphane-3,5-dione (50 mg, 121 μmol) in dioxane (2.0 ml) at rt. The reaction mixture was purged with nitrogen for 15 minutes and then CuI (69 mg, 362 μmol) followed by 1,2-dimethylethylenediamine (43 mg, 483 μmol) were added. The reaction mixture was stirred at 110° C. for 16 h. The reaction mixture was filtered through a celite bed using 10% CH₂Cl₂-MeOH. The organic layer was washed with water, dried (Na₂SO₄) and concentrated under reduced pressure. The crude product was purified by reverse phase HPLC (ammonium acetate mobile phase) to give (S,E)-1⁴-(3-cyclopropyl-2-oxoimidazolidin-1-yl)-11-methyl-1²,1³-dihydro-1¹H-2,6-diaza-1(2,3)-imidazo[4,5-c]pyridina-4(1,3)-benzenacycloundecaphane-3,5-dione (9 mg, 15%).

¹H NMR (DMSO-d₆) @ 353.2 K δ: 8.85 (bs, 1H), 9.17 (d, 1H), 8.11 (d, 1H), 7.77 (d, 1H), 7.59 (m, 1H), 7.51 (bs, 1H), 7.42 (d, 1H), 5.08 (bs, 1H), 3.10 (m, 1H), 1.93 (m, 3H), 0.71 (s, 4H); LC/MS: M+H=488.40.

### Example 123: (S,E)-1⁵-(2-hydroxypropan-2-yl)-N,N,11-trimethyl-3,5-dioxo-1²,1³-dihydro-1¹H-2,6-diaza-1(2,1)-benzo[d]imidazola-4(1,3)-benzenacycloundecaphane-1⁷-carboxamide

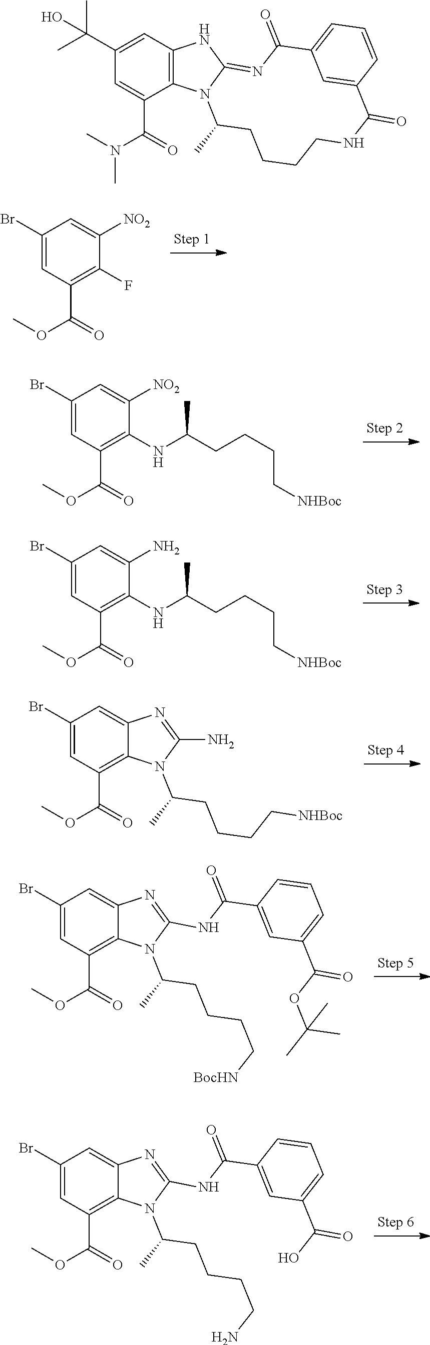

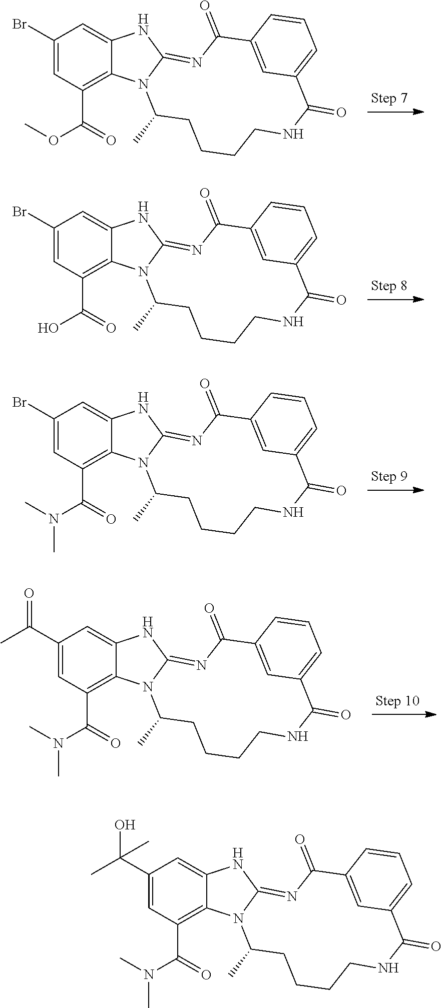

### Step 1: Preparation of methyl (S)-5-bromo-2-((6-((tert-butoxycarbonyl)amino)hexan-2-yl)amino)-3-nitrobenzoate

To a stirred solution of methyl 5-bromo-2-fluoro-3-nitrobenzoate (15 g, 54 mmol) and tert-butyl (S)-(5-aminohexyl)carbamate (15.2 g, 70.1 mmol) in DMF (50 ml), was added N,N-diisopropylethylamine (47.8 ml, 270 mmol). The reaction mixture was stirred at 80° C. for 16 h. The reaction mixture was diluted with water and extracted with EtOAc. The organic layer was concentrated under reduced pressure and the crude product was purified by flash chromatography (SiO₂, 20 to 50% EtOAc in heptane) to afford methyl (S)-5-bromo-2-((6-((tert-butoxycarbonyl)amino)hexan-2-yl)amino)-3-nitrobenzoate (20.1 g, 76%).

LC/MS: M+H=475.90; Rt=2.44 min.

### Step 2: Preparation of methyl (S)-3-amino-5-bromo-2-((6-((tert-butoxycarbonyl)amino)hexan-2-yl)amino)benzoate

To a stirred solution of (S)-5-bromo-2-((6-((tert-butoxycarbonyl)amino)hexan-2-yl)amino)-3-nitrobenzoate (20 g, 41.3 mmol) in EtOAc (408 ml) was added nickel (9.7 g, 165 mmol). The reaction mixture was tired under a hydrogen atmosphere for 4 h. The reaction mixture was filtered through celite and the filtrate was concentrated under reduced pressure. The crude product (16.2 g) was used without further purification.

LC/MS: M+H=445.75; Rt=2.37 min.

### Step 3: Preparation of methyl (S)-2-amino-5-bromo-1-(6-((tert-butoxycarbonyl)amino)hexan-2-yl)-1H-benzo[d]imidazole-7-carboxylate

To a stirred solution of methyl (S)-3-amino-5-bromo-2-((6-((tert-butoxycarbonyl)amino)hexan-2-yl)amino)benzoate (16.2 g, 30.6 mmol) and cyanogen bromide (3.89 g, 36.7 mmol) in MeCN (40 ml) and MeOH (120 ml) was added water (40 ml). The reaction mixture was stirred at 55° C. for 3 h. The reaction mixture was concentrated under reduced pressure. The residue was dissolved in water and extracted with EtOAc. The organic layer was concentrated under reduced pressure and the crude product was purified by flash chromatography (SiO₂, 0-10% MeOH in CH₂Cl₂) to afford of methyl (S)-2-amino-5-bromo-1-(6-((tert-butoxycarbonyl)amino)hexan-2-yl)-1H-benzo[d]imidazole-7-carboxylate (13.6 g, 76%).

¹H NMR (DMSO-d₆) δ: 7.41 (d, 1H), 7.24 (d, 1H), 6.68 m, 1H), 6.58 (bs, 1H), 3.89 (s, 3H), 2.78 (m, 1H), 1.46 (d, 3H), 1.28 (s, 9H), 1.26 (m, 2H); LC/MS: M+H=470.75.

### Step 4: Preparation of methyl (S)-5-bromo-1-(6-((tert-butoxycarbonyl)amino)hexan-2-yl)-2-(3-(tert-butoxycarbonyl)benzamido)-1H-benzo[d]imidazole-7-carboxylate

To a solution of 3-(tert-butoxycarbonyl)benzoic acid (7.82 g, 35.2 mmol) in MeCN (140 ml) at 0° C. was added TCFH (13.2 g, 46.9 mmol) and 1-methyl-1H-imidazole (9.35 ml, 117 mmol). The solution was stirred at 0° C. for 10 min and then a solution of methyl (S)-2-amino-5-bromo-1-(6-((tert-butoxycarbonyl)amino)hexan-2-yl)-1H-benzo[d]imidazole-7-carboxylate (13.6 g, 23.5 mmol) in MeCN (20 ml) was added. The resulting solution was stirred at rt for 16 h. The reaction mixture was concentrated under reduced pressure and ice-cold water was added. The mixture was extracted with EtOAc. The organic layer was washed with brine, dried (Na₂SO₄) and concentrated under reduced pressure. The crude was purified by flash chromatography (SiO₂, 50-70% EtOAc in heptane) to afford methyl (S)-5-bromo-1-(6-((tert-butoxycarbonyl)amino)hexan-2-yl)-2-(3-(tert-butoxycarbonyl)benzamido)-1H-benzo[d]imidazole-7-carboxylate (14.9 g, 92%).

¹H NMR (DMSO-d₆) δ: 8.81 (s, 1H), 8.37 (d, 1H), 8.06 (d, 1H), 7.88 (m, 1H), 7.64 (m, 2H), 6.63 (m, 1H), 4.49 (bs, 1H), 4.02 (m, 1H), 3.95 (s, 3H), 2.77 (m, 2H), 1.73 (d, 3H), 1.29 (s, 9H); LC/MS: M+H=674.85.

### Step 5: Preparation of (S)-3-((1-(6-aminohexan-2-yl)-5-bromo-7-(methoxycarbonyl)-1H-benzo[d]imidazol-2-yl)carbamoyl)benzoic acid

A solution of methyl (S)-5-bromo-1-(6-((tert-butoxycarbonyl)amino)hexan-2-yl)-2-(3-(tert-butoxycarbonyl)benzamido)-1H-benzo[d]imidazole-7-carboxylate (14.8 g, 21.5 mmol) in TFA (36.3 ml) was stirred at 0° C. for 16 h. The reaction mixture was concentrated under reduced pressure and the residue was triturated with diethyl ether. The product (10.8 g) was used without further purification.

¹H NMR (DMSO-d₆) δ: 8.79 (s, 1H), 8.40 (d, 1H), 8.11 (d, 1H), 7.91 (d, 1H), 7.65 (m, 2H), 4.53 (bs, 1H), 3.96 (s, 3H), 2.67 (m, 2H), 1.98 (m, 1H), 1.72 (d, 3H), 1.46 (m, 2H), 1.20 (m, 1H), 0.98 (m, 1H); LC/MS: M+H=519.20.

### Step 6: Preparation of methyl (S,E)-1⁵-bromo-11-methyl-3,5-dioxo-1²,1³-dihydro-1¹H-2,6-diaza-1(2,1)-benzo[d]imidazola-4(1,3)-benzenacycloundecaphane-1⁷-carboxylate

A solution of TCFH (6.31 g, 22.5 mmol) and 1-methyl-1H-imidazole (4.48 ml, 56.2 mmol) in MeCN (4 l) was stirred at 0° C. for 15 min. To this solution was added slowly a solution of (S)-3-((1-(6-aminohexan-2-yl)-5-bromo-7-(methoxycarbonyl)-1H-benzo[d]imidazol-2-yl)carbamoyl)benzoic acid (6.0 g, 11.2 mmol) in DMF (10 ml) and MeCN (4 l). The resulting reaction mixture was stirred at 0° C. for 16 h. The reaction mixture was quenched with water (50 ml) and concentrated under reduced pressure. The crude residue was diluted with water. The formed precipitate was filtered, washed with cold water and dried in vacuum. The crude product was triturated with acetonitrile and filtered to afford methyl (S,E)-1⁵-bromo-11-methyl-3,5-dioxo-1²,1³-dihydro-1¹H-2,6-diaza-1(2,1)-benzo[d]imidazola-4(1,3)-benzenacycloundecaphane-1⁷-carboxylate (1.8 g, 30%).

¹H NMR (DMSO-d₆) δ: 8.80 (s, 1H), 8.11 (d, 1H), 7.78 (m, 3H), 7.60 (m, 2H), 3.93 (s, 3H), 1.89 (d, 3H), 1.59 (m, 3H); LC/MS: M+H=501.15.

### Step 7: Preparation of (S,E)-15-bromo-11-methyl-3,5-dioxo-1²,1³-dihydro-1¹H-2,6-diaza-1(2,1)-benzo[d]imidazola-4(1,3)-benzenacycloundecaphane-1⁷-carboxylic acid

To a solution of methyl (S,E)-1⁵-bromo-11-methyl-3,5-dioxo-1²,1³-dihydro-1¹H-2,6-diaza-1(2,1)-benzo[d]imidazola-4(1,3)-benzenacycloundecaphane-1⁷-carboxylate (1.8 g, 3.42 mmol) in dioxane (30 ml) and water (10 ml) was added LiOH (492 mg, 20.5 mmol). The resulting reaction mixture was stirred at rt for 16 h. The reaction mixture was diluted with water and acidified with 2M HCl. The formed precipitate was filtered and dried in vacuum. The crude product (1.6 g) was used without further purification.

¹H NMR (DMSO-d₆) δ: 8.79 (s, 1H), 8.10 (m, 1H), 7.78 (m, 2H), 7.60 (m, 2H), 1.90 (m, 3H), 1.75 (m, 3H), 1.69 (m, 2H), 1.63 (m, 3H); LC/MS: M+H=486.80.

### Step 8: Preparation of (S,E)-1⁵-bromo-N,N,11-trimethyl-3,5-dioxo-1²,1³-dihydro-1¹H-2,6-diaza-1(2,1)-benzo[d]imidazola-4(1,3)-benzenacycloundecaphane-1⁷-carboxamide

To a solution of (S,E)-1⁵-bromo-11-methyl-3,5-dioxo-1²,1³-dihydro-1¹H-2,6-diaza-1(2,1)-benzo[d]imidazola-4(1,3)-benzenacycloundecaphane-1⁷-carboxylic acid (1.6 g, 2.97 mmol) MeCN (60 ml) and DMF (10 ml) was added TCFH (1.66 g, 5.93 mmol) 1-methyl-1H-imidazole (1.18 ml, 14.8 mmol). The solution was stirred at rt for 15 min and then N,N′-dimethyl amine (554 μl, 14.8 mmol) was added dropwise. The resulting solution was stirred at rt for 16 h. The reaction mixture was concentrated under reduced pressure. The residue was triturated with water and then purified by flash chromatography (SiO₂, 10% MeOH/EtOAc 2:3 in CH₂Cl₂) to afford (S,E)-1⁵-bromo-N,N,11-trimethyl-3,5-dioxo-1²,1³-dihydro-1¹H-2,6-diaza-1(2,1)-benzo[d]imidazola-4(1,3)-benzenacycloundecaphane-1⁷-carboxamide (1.5 g, 93%).

¹H NMR (DMSO-d₆) δ: 8.10 (d, 1H), 7.88 (m, 1H), 7.75 (d, 1H), 7.60 (m, 1H), 7.27 (s, 1H), 3.05 (m, 3H), 2.87 (s, 3H); LC/MS: M+H=513.75.

### Step 9: Preparation of (S,E)-1⁵-acetyl-N,N,11-trimethyl-3,5-dioxo-1²,1³-dihydro-1¹H-2,6-diaza-1(2,1)-benzo[d]imidazola-4(1,3)-benzenacycloundecaphane-1⁷-carboxamide

A solution of (S,E)-1⁵-bromo-N,N,11-trimethyl-3,5-dioxo-1²,1³-dihydro-1¹H-2,6-diaza-1(2,1)-benzo[d]imidazola-4(1,3)-benzenacycloundecaphane-1⁷-carboxamide (250 mg, 463 μmol), tributyl(1-ethoxyethenyl)stannane (335 mg, 927 μmol), LiCl (58.9 mg, 1.39 mmol) and palladium bis(triphenylphosphine) dichloride (65.1 mg, 92.7 μmol) in DMF (5.0 ml) was degassed with nitrogen and then heated in a sealed tube at 120° C. for 16 h. The reaction mixture was cooled, diluted with water, and extracted with 5% MeOH in CH₂Cl₂. The organic layer was concentrated under reduced pressure and the residue was purified by flash chromatography (SiO₂, 10% MeOH in CH₂Cl₂) to afford (S,E)-1⁵-acetyl-N,N,11-trimethyl-3,5-dioxo-1²,1³-dihydro-1¹H-2,6-diaza-1(2,1)-benzo[d]imidazola-4(1,3)-benzenacycloundecaphane-1⁷-carboxamide (110 mg, 44%).

¹H NMR (DMSO-d₆) δ: 8.77 (bs, 1H), 8.11 (m, 2H), 7.77 (m, 2H), 7.61 (m, 2H), 3.09 (s, 3H), 2.89 (s, 3H), 2.60 (s, 3H), 1.69 (m, 2H), 1.57 (m, 2H); LC/MS: M+H=476.30.

### Step 10: Preparation of (S,E)-15-(2-hydroxypropan-2-yl)-N,N,11-trimethyl-3,5-dioxo-1²,1³-dihydro-1¹H-2,6-diaza-1(2,1)-benzo[d]imidazola-4(1,3)-benzenacycloundecaphane-1⁷-carboxamide

3 M MeMgBr in diethyl ether (1.89 mmol, 631 μl) was added dropwise to a solution of (S,E)-1⁵-acetyl-N,N,11-trimethyl-3,5-dioxo-1²,1³-dihydro-1¹H-2,6-diaza-1(2,1)-benzo[d]imidazola-4(1,3)-benzenacycloundecaphane-1⁷-carboxamide (100 mg, 189 μmol) in dry THF at 0° C. The reaction mixture was stirred at 0° C. for 1 h and then quenched by the addition of saturated NH₄Cl₂. The aqueous solution was extracted with 5% MeOH in CH₂Cl₂. The organic layer was concentrated under reduced pressure and the crude compound was purified by prep HPLC to afford (S,E)-1⁵-(2-hydroxypropan-2-yl)-N,N,11-trimethyl-3,5-dioxo-1²,1³-dihydro-1¹H-2,6-diaza-1(2,1)-benzo[d]imidazola-4(1,3)-benzenacycloundecaphane-1⁷-carboxamide (21 mg, 22%).

¹H NMR (DMSO-d₆) @ 353.2 K δ: 8.88 (s, 1H), 8.10 (d, 1H), 7.75 (d, 1H), 7.71 (s, 1H), 7.57 (m, 1H), 7.50 (m, 1H), 7.15 (m, 1H), 4.89 (s, 1H), 3.46 (bs, 1H), 3.09 (s, 3H), 2.88 (s, 3H), 1.60 (d, 3H), 1.49 (s, 6H); LC/MS: M+H=492.35.

### Example 124: (S,E)-N,N,11-trimethyl-1⁵-((4-methylpiperazin-1-yl)methyl)-3,5-dioxo-1²,1³-dihydro-1¹H-2,6-diaza-1(2,1)-benzo[d]imidazola-4(1,3)-benzenacycloundecaphane-1⁷-carboxamide

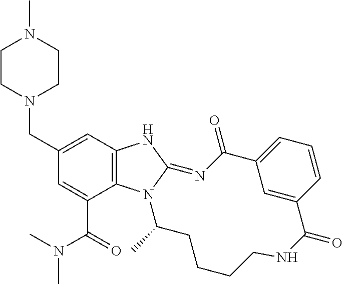 [SMILES: C[C@H]1CCCCNC(=O)c2cccc(c2)C(=O)/N=C2\Nc3cc(CN4CCN(C)CC4)cc(C(=O)N(C)C)c3N21]

A solution of (S,E)-1⁵-bromo-N,N,11-trimethyl-3,5-dioxo-1²,1³-dihydro-1¹H-2,6-diaza-1(2,1)-benzo[d]imidazola-4(1,3)-benzenacycloundecaphane-1⁷-carboxamide (100 mg, 185 μmol), potassium trifluoro((4-methylpiperazin-1-yl)methyl)borate (82 mg, 371 μmol), Cs₂CO₃ (242 mg, 742 μmol), xPhos (26.5 mg, 55.6 μmol) and Pd(OAc)₂ (4.16 mg, 18.5 μmol) in dioxane (4 ml) and water (1 ml) was heated in a sealed tube at 120° C. for 16 h. The reaction mixture was cooled, diluted with water and extracted with 5% MeOH in CH₂Cl₂. The organic layer was concentrated under reduced pressure and the residue preparative HPLC to afford (S,E)-N,N,11-trimethyl-1⁵-((4-methylpiperazin-1-yl)methyl)-3,5-dioxo-1²,1³-dihydro-1¹H-2,6-diaza-1(2,1)-benzo[d]imidazola-4(1,3)-benzenacycloundecaphane-1⁷-carboxamide (16 mg, 16%).

¹H NMR (DMSO-d₆) @ 353.2 K δ: 8.86 (bs, 1H), 8.10 (d, 1H), 7.76 (d, 1H), 7.58 (m, 1H), 7.51 (m, 2H), 7.03 (s, 1H), 4.72 (bs, 1H), 3.71 (s, 2H), 3.09 (s, 4H), 2.89 (s, 3H), 2.67 (s, 2H), 1.89 (m, 3H), 1.60 (m, 3H); LC/MS: M+H=546.45.

### Example 125: (S,E)-N,N,11-trimethyl-1⁵-(morpholinomethyl)-3,5-dioxo-1²,1³-dihydro-1¹H-2,6-diaza-1(2,1)-benzo[d]imidazola-4(1,3)-benzenacycloundecaphane-1⁷-carboxamide

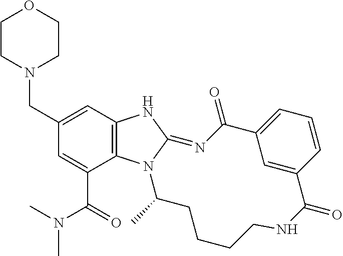 [SMILES: C[C@H]1CCCCNC(=O)c2cccc(c2)C(=O)/N=C2\Nc3cc(CN4CCOCC4)cc(C(=O)N(C)C)c3N21]

Example 125 was prepared from (S,E)-1⁵-bromo-N,N,11-trimethyl-3,5-dioxo-1²,1³-dihydro-1¹H-2,6-diaza-1(2,1)-benzo[d]imidazola-4(1,3)-benzenacycloundecaphane-1⁷-carboxamide and potassium trifluoro(morpholinomethyl)borate analogous to the preparation of Example 124.

¹H NMR (DMSO-d₆) @ 353.2 K δ: 8.88 (bs, 1H), 8.10 (d, 1H), 7.75 (d, d, 1H), 7.57 (m, 1H), 7.52 (m, 2H), 6.99 (s, 1H), 3.62 (m, 4H), 3.55 (s, 2H), 2.87 (s, 3H), 2.49 (m, 4H), 1.46 (d, 3H); LC/MS: M+H=533.35.

### Examples 126 and 127: (11S,E)-1⁴-(3-fluoro-2-oxopyrrolidin-1-yl)-11-methyl-1²,1³-dihydro-1¹H-2,6-diaza-1(2,3)-imidazo[4,5-c]pyridina-4(1,3)-benzenacycloundecaphane-3,5-dione

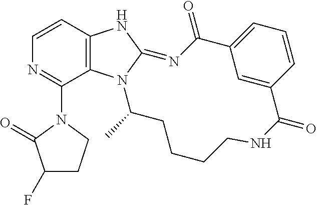 [SMILES: C[C@H]1CCCCNC(=O)c2cccc(c2)C(=O)/N=C2\Nc3ccnc(N4CCC(F)C4=O)c3N21]

To a stirred solution of (S,E)-1⁴-chloro-11-methyl-1²,1³-dihydro-1¹H-2,6-diaza-1(2,3)-imidazo[4,5-c]pyridina-4(1,3)-benzenacycloundecaphane-3,5-dione (100 mg, 239 μmol) in dioxane (2.85 ml), was added Cs₂CO₃ (233 mg, 716 μmol) and 3-fluoropyrrolidin-2-one (27.1 mg, 263 μmol). The reaction mixture was purged with nitrogen for 15 minutes and then CuI (136 mg, 239 μmol) and methyl[2-(methylamino)ethyl]amine (84.2 mg, 955 μmol) were added. The reaction mixture was stirred at 110° C. for 16 h. The reaction mixture was filtered through celite using 10% MeOH/CH₂Cl₂. The organic layer washed with water and the combined organic layer was dried (Na₂SO₄) and was concentrated under reduced pressure: The crude was stepwise purified by preparative HPLC and SFC to give separated diastereomers.

### Example 126, Diastereomer 1

¹H NMR (DMSO-d₆) @ 353.2 K δ: 8.83 (bs, 1H), 8.26 (d, 1H), 8.11 (d, 1H), 7.77 (d, 1H), 7.59 (m, 1H), 7.51 (d, 2H), 5.40 (m, 1H), 4.72 (m, 1H), 3.54 (m, 1H), 2.70 (m, 1H), 1.65 (d, 3H); LC/MS: M+H=465.30.

Xtimate Ethylpyridine (250*4.6) mm, 5 um, 0.1% TEA in MeOH, Flow Rate: 1.0 mL/min, Rt=2.66 min

### Example 127, Diastereomer 2

¹H NMR (DMSO-d₆) @ 353.2 K δ: 8.84 (m, 1H), 8.25 (m, 1H), 7.76 (m, 1H), 7.59 (m, 1H), 7.51 (m, 2H), 1.66 (d, 3H); LC/MS: M+H=465.35.

Xtimate Ethylpyridine (250*4.6) mm, 5 um, 0.1% TEA in MeOH, Flow Rate: 1.0 mL/min, Rt=3.25 min

### Example 128: (S,E)-N,N,11-trimethyl-3,5-dioxo-1²,1³-dihydro-1¹H-2,6-diaza-1(2,3)-imidazo[4,5-c]pyridina-4(1,3)-benzenacycloundecaphane-1⁴-carboxamide

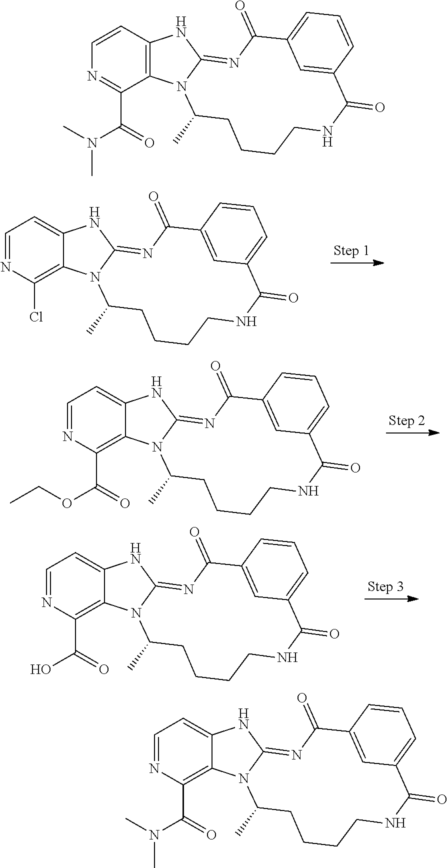

### Step 1: Preparation of ethyl (S,E)-11-methyl-3,5-dioxo-1²,1³-dihydro-1¹H-2,6-diaza-1(2,3)-imidazo[4,5-c]pyridina-4(1,3)-benzenacycloundecaphane-1⁴-carboxylate

KOAc (142 mg, 1.44 mmol) was added to a solution of (S,E)-1⁴-chloro-11-methyl-1²,1³-dihydro-1¹H-2,6-diaza-1(2,3)-imidazo[4,5-c]pyridina-4(1,3)-benzenacycloundecaphane-3,5-dione (50 mg, 121 μmol) in ethanol (77 ml) in a steel vessel. The reaction mixture was purged with nitrogen for 15 minutes and then Pd(dppf)Cl₂ (59 mg, 72 μmol) was added. The reaction vessel was filled with CO (100 psi) and then stirred at 110° C. for 16 h. The reaction mixture was filtered through a celite bed using 10% CH₂Cl₂-MeOH. The organic layer was washed with water, dried (Na₂SO₄) and concentrated under reduced pressure. The crude product was purified by flash chromatography (SiO₂, 10% MeOH in CH₂Cl₂) to afford ethyl (S,E)-11-methyl-3,5-dioxo-1²,1³-dihydro-1¹H-2,6-diaza-1(2,3)-imidazo[4,5-c]pyridina-4(1,3)-benzenacycloundecaphane-1⁴-carboxylate (180 mg, 78%).

¹H NMR (DMSO-d₆) δ: 8.84 (bs, 1H), 8.35 (d, 1H), 8.13 (d, 1H), 7.85 (m, 1H), 7.78 (d, 1H), 7.59 (m, 2H), 5.76 (s, 1H), 4.44 (m, 2H), 1.60 (d, 3H), 1.36 (m, 3H); LC/MS: M+H=435.95.

### Step 2: Preparation of (S,E)-11-methyl-3,5-dioxo-1²,1³-dihydro-1¹H-2,6-diaza-1(2,3)-imidazo[4,5-c]pyridina-4(1,3)-benzenacycloundecaphane-1⁴-carboxylic acid

LiOH (42 mg, 1.0 mmol) was added to a solution of ethyl (S,E)-11-methyl-3,5-dioxo-1²,1³-dihydro-1¹H-2,6-diaza-1(2,3)-imidazo[4,5-c]pyridina-4(1,3)-benzenacycloundecaphane-1⁴-carboxylate (1200 mg, 251 μmol) in THF (5 ml) and water (2 ml). The reaction mixture was stirred at rt for 16 h. The reaction mixture was concentrated under reduced pressure. The reaction mixture was acidified with 2 M HCl. The formed precipitate was collected and dried under reduced pressure. The product was used without further purification.

¹H NMR (DMSO-d₆) δ: 8.85 (bs, 1H), 8.33 (d, 1H), 8.11 (bs, 1H), 7.85 (m, 2H), 7.60 (m, 2H), 5.27 (m, 1H), 2.22 (m, 1H), 1.64 (d, 3H).

### Step 3: Preparation of (S,E)-N,N,11-trimethyl-3,5-dioxo-1²,1³-dihydro-1¹H-2,6-diaza-1(2,3)-imidazo[4,5-c]pyridina-4(1,3)-benzenacycloundecaphane-1⁴-carboxamide

TCFH (47 mg, 167 μmol) was added to a solution of (S,E)-11-methyl-3,5-dioxo-1²,1³-dihydro-1¹H-2,6-diaza-1(2,3)-imidazo[4,5-c]pyridina-4(1,3)-benzenacycloundecaphane-1⁴-carboxylic acid (85 mg, 83 μmol) and 1-methyl-1H-imidazole (33.3 μl, 417 μmol) in DMF (1 ml) and MeCN (5 ml). The reaction mixture was stirred at rt for 15 min. 2M N,N-dimethyl amine in THF (469 μl, 939 μmol) was added dropwise. The reaction mixture was stirred at rt for 4 h. The reaction mixture was concentrated under reduced pressure and extracted with 10% MeOH/CH₂Cl₂. The organic layer was washed with brine solution, dried over Na₂SO₄ and concentrated under reduced pressure. The crude was purified by prep HPLC to afford (S,E)-N,N,11-trimethyl-3,5-dioxo-1²,1³-dihydro-1¹H-2,6-diaza-1(2,3)-imidazo[4,5-c]pyridina-4(1,3)-benzenacycloundecaphane-1⁴-carboxamide (5 mg, 6%).

¹H NMR (DMSO-d₆) @ 351.3 K δ: 8.83 (bs, 1H), 8.30 (d, 1H), 8.10 (d, 1H), 7.76 (d, 1H), 7.59 (m, 1H), 7.49 (d, 1H), 4.85 (bs, 1H), 3.14 (m, 1H), 3.07 (s, 3H), 2.85 (s, 3H), 1.89 (m, 3H), 1.57 (d, 3H), 1.44 (m, 1H); LC/MS: M+H=435.35.

### Example 129: (S,E)-11-methyl-1⁷-(1-(oxetan-3-yl)-1H-1,2,3-triazol-4-yl)-1²,1³-dihydro-1¹H-2,6-diaza-1(2,1)-benzo[d]imidazola-4(1,3)-benzenacycloundecaphane-3,5-dione

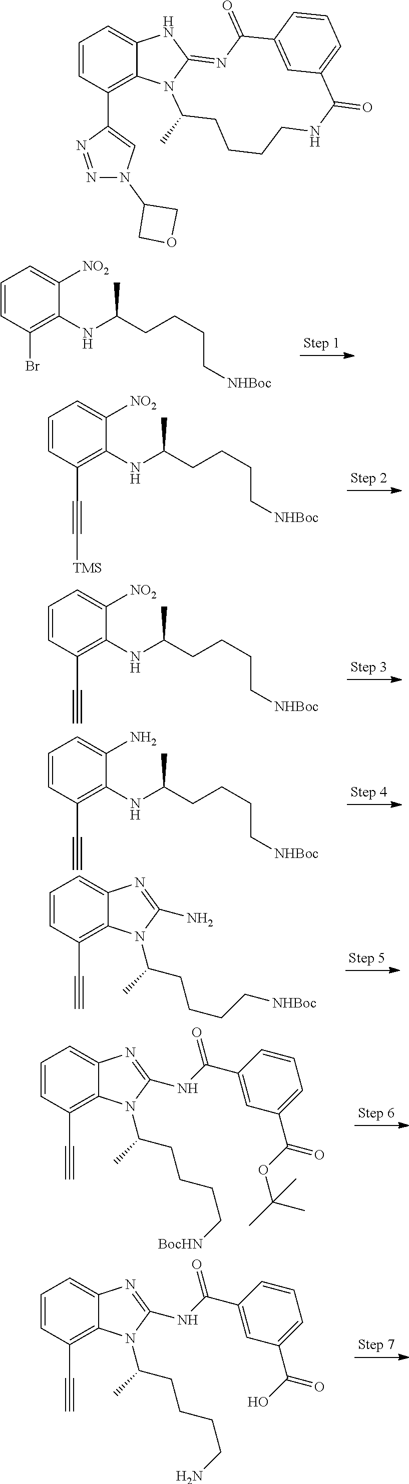

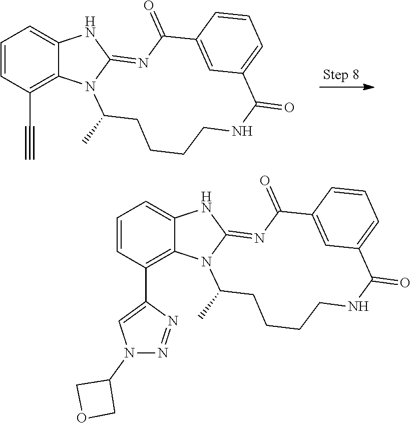 [SMILES: C#Cc1cccc2c1N1/C(=N/C(=O)c3cccc(c3)C(=O)NCCCC[C@@H]1C)N2 ; C[C@H]1CCCCNC(=O)c2cccc(c2)C(=O)/N=C2\Nc3cccc(-c4cn(C5COC5)nn4)c3N21]

### Step 1: Preparation of tert-butyl (S)-(5-((2-nitro-6-((trimethylsilyl)ethynyl)phenyl)amino)hexyl)carbamate

A solution of tert-butyl (S)-(5-((2-bromo-6-nitrophenyl)amino)hexyl)carbamate (6 g, 14.4 mmol), TMS-acetylene (3.03 ml, 21.6 mmol) and TEA (6.25 ml, 43.2 mmol) in MeCN (50 ml) was purged with argon for 10 min. To the reaction solution was added bis(triphenylphosphine)palladium(II) dichloride (506 mg, 721 μmol) and CuI (274 mg, 21.6 mmol). The reaction mixture was stirred at 80° C. for 16 h. The reaction mixture was concentrated under reduced pressure. The crude was purified by flash chromatography (SiO₂, 0-30% EtOAC/heptane v/v) to give tert-butyl (S)-(5-((2-nitro-6-((trimethylsilyl)ethynyl)phenyl)amino)hexyl)carbamate (4.9 g, 65%)

¹H NMR (DMSO-d₆) δ: 8.08 (d, 1H), 7.63 (m, 1H), 6.75 (m, 1H), 4.75 (m, 1H), 2.86 (m, 2H), 1.51 (m, 2H), 1.35 (s, 9H), 1.18 (d, 3H), 0.234 (s, 9H); LC/MS: M+H=434.60.

### Step 2: Preparation of tert-butyl (S)-(5-((2-ethynyl-6-nitrophenyl)amino)hexyl)carbamate

A solution of tert-butyl (S)-(5-((2-nitro-6-((trimethylsilyl)ethynyl)phenyl)amino)hexyl)carbamate (4.9 g, 11.3 mmol) and K₂CO₃ (3.12 g, 22.6 mmol) in MeOH (40 ml) and diethyl ether (20 ml) was stirred at rt for 3 h. The reaction mixture was quenched with water. The aqueous layer was extracted with EtOAc, dried (Na₂SO₄) and concentrated under reduced pressure. The crude residue was purified by flash chromatography (SiO₂, 0-10% EtOAC/heptane v/v) to give tert-butyl (S)-(5-((2-ethynyl-6-nitrophenyl)amino)hexyl)carbamate (2.8, 64%).

¹H NMR (DMSO-d₆) δ: 8.07 (m, 1H), 7.68 (m, 1H), 7.49 (d, 1H), 6.76 (m, 2H), 4.59 (m, 2H), 2.86 (m, 2H), 1.51 (m, 3H), 1.39 (s, 9H), 1.19 (d, 3H), 0.82 (m, 2H); LC/MS: M+H=362.10.

### Step 3: Preparation of tert-butyl (S)-(5-((2-amino-6-ethynylphenyl)amino)hexyl)carbamate

A solution of tert-butyl (S)-(5-((2-ethynyl-6-nitrophenyl)amino)hexyl)carbamate (2.8 g, 7.75 mmol), ammonium chloride (4.14 g, 77.5 mmol) and zinc (5.06 g, 77.5 mmol) EtOH (70 ml) and water (21 ml) was stirred at rt 16 h. The reaction mixture was concentrated under reduced pressure. The crude residue was diluted with water. The aqueous layer was extracted with EtOAc, dried (Na₂SO₄) and concentrated reduced pressure. The crude residue was purified by flash chromatography (SiO₂, 0-10% EtOAC/MeOH v/v) to give tert-butyl (S)-(5-((2-amino-6-ethynylphenyl)amino)hexyl)carbamate (2.3 g, 74%)

¹H NMR (DMSO-d₆) δ: 6.74 (m, 1H), 6.63 (m, 2H), 4.66 (s, 1H), 3.51 (m, 1H), 2.89 (m, 1H), 1.37 (s, 9H), 0.98 (m, 1H); LC/MS: M+H=332.20.

### Step 4: Preparation of tert-butyl (S)-(5-(2-amino-7-ethynyl-1H-benzo[d]imidazol-1-yl)hexyl)carbamate

A solution of tert-butyl (S)-(5-((2-amino-6-ethynylphenyl)amino)hexyl)carbamate (2.3 g, 6.94 mmol) and cyanogen bromide (882 mg, 8.33 mmol) in MeOH (20 ml), MeCN (10 ml) and water (10 ml) was heated at 55° C. for 2 h. The reaction mixture was concentrated under reduced pressure. The crude residue was diluted with water (50 ml) and the aqueous layer was extracted with Et₂O (2×10 ml). The aqueous layer was basified with excess NaHCO₃. The aqueous layer was extracted with 5% MeOH in CH₂Cl₂, dried (Na₂SO₄) and concentrated under reduced pressure. The product was used without further purification.

¹H NMR (DMSO-d₆) δ: 7.16 (d, 1H), 6.94 (m, 2H), 6.71 (bs, 1H), 6.24 (bs, 1H), 4.43 (bs, 1H), 2.82 (m, 2H), 1.46 (m, 2H), 1.34 (s, 9H); LC/MS: M+H=357.30.

### Step 5: Preparation of tert-butyl (S)-3-((1-(6-((tert-butoxycarbonyl)amino)hexan-2-yl)-7-ethynyl-1H-benzo[d]imidazol-2-yl)carbamoyl)benzoate

To a solution of 3-(tert-butoxycarbonyl)benzoic acid (1.7 g, 7.66 mmol) and 1-methyl-1H-imidazole (1.41 ml, 17.7 mmol) in MeCN (30 ml) was added TCFH (2.64 g, 9.43 mmol) at rt. To this reaction mixture was added tert-butyl (S)-(5-(2-amino-7-ethynyl-1H-benzo[d]imidazol-1-yl)hexyl)carbamate (2.1 g, 5.89 mmol). The reaction mixture was stirred at rt for 16 h and then concentrated under reduced pressure. The crude residue was diluted with cold water. The precipitate formed was filtered, washed with cold water and dried under reduced pressure. The crude residue was purified by flash chromatography (SiO₂, 0 to 80% EtOAc in heptane) to give tert-butyl (S)-3-((1-(6-((tert-butoxycarbonyl)amino)hexan-2-yl)-7-ethynyl-1H-benzo[d]imidazol-2-yl)carbamoyl)benzoate (2.3 g, 49%).

¹H NMR (DMSO-d₆) δ: 8.82 (s, 1H), 8.36 (d, 1H), 8.04 (d, 1H), 7.63 (m, 2H), 7.35 (d, 1H), 7.20 (d, 1H), 5.79 (m, 1H), 4.73 (s, 1H), 2.81 (m, 2H), 1.72 (d, 3H), 1.51 (s, 9H), 1.29 (s, 9H); LC/MS: M+H=561.50.

### Step 6: Preparation of (S)-3-((1-(6-aminohexan-2-yl)-7-ethynyl-1H-benzo[d]imidazol-2-yl)carbamoyl)benzoic acid

To a stirred solution of tert-butyl (S)-3-((1-(6-((tert-butoxycarbonyl)amino)hexan-2-yl)-7-ethynyl-1H-benzo[d]imidazol-2-yl)carbamoyl)benzoate (2.3 g, 4.1 mmol) in CH₂Cl₂ (20 ml) was added dropwise TFA (3.14 ml, 41 mmol) at rt. The reaction mixture was stirred at rt for 16 h. The reaction mixture was concentrated under reduced pressure and the crude residue was triturated with Et₂O to give (S)-3-((1-(6-aminohexan-2-yl)-7-ethynyl-1H-benzo[d]imidazol-2-yl)carbamoyl)benzoic acid (1.53 g, 92%).

¹H NMR (DMSO-d₆) δ: 8.79 (s, 1H), 8.40 (d, 1H), 8.10 (d, 1H), 7.63 (m, 2H), 7.37 (d, 1H), 7.22 (m, 1H), 4.75 (s, 1H), 2.71 (m, 3H), 1.97 (m, 1H), 1.72 (d, 3H), 1.53 (m, 2H), 1.36 (m, 1H); LC/MS: M+H=405.10.

### Step 7: Preparation of (S,E)-1⁷-ethynyl-11-methyl-1²,1³-dihydro-1¹H-2,6-diaza-1(2,1)-benzo[d]imidazola-4(1,3)-benzenacycloundecaphane-3,5-dione

A solution of 1-methyl-1H-imidazole (1.04 ml, 13.1 mmol) and TCFH (1.53 g, 5.44 mmol) in MeCN (3000 ml) was cooled to 0° C. and stirred for 15 min. A solution of (S)-3-((1-(6-aminohexan-2-yl)-7-ethynyl-1H-benzo[d]imidazol-2-yl)carbamoyl)benzoic acid (2.0 g, 4.94 mmol) in MeCN (3000 ml) and DMF (50 ml) at rt. The reaction mixture was stirred at 0° C. for 4 h. The reaction mixture was quenched with water and concentrated under reduced pressure. The crude product was triturated with water and the precipitate was collected by filtration. The product was purified by flash chromatography (SiO₂, 0 to 10% MeOH in CH₂Cl₂) to give (S,E)-1⁷-ethynyl-11-methyl-1²,1³-dihydro-1¹H-2,6-diaza-1(2,1)-benzo[d]imidazola-4(1,3)-benzenacycloundecaphane-3,5-dione (620 mg, 73%).

¹H NMR (DMSO-d₆) δ: 8.10 (d, 1H), 7.75 (d, 1H), 7.59 (m, 2H), 7.36 (m, 1H), 7.21 (m, 1H), 5.75 (s, 1H), 4.65 (bs, 1H), 1.75 (d, 3H); LC/MS: M+H=387.10.

### Step 8: Preparation of (S,E)-11-methyl-1⁷-(1-(oxetan-3-yl)-1H-1,2,3-triazol-4-yl)-1²,1³-dihydro-1¹H-2,6-diaza-1(2,1)-benzo[d]imidazola-4(1,3)-benzenacycloundecaphane-3,5-dione

A solution of (S,E)-1⁷-ethynyl-11-methyl-1²,1³-dihydro-1¹H-2,6-diaza-1(2,1)-benzo[d]imidazola-4(1,3)-benzenacycloundecaphane-3,5-dione, 3-azidooxetane (154 mg, 1.55 mmol), sodium ascorbate (51 mg, 259 μmol) and copper sulfate pentahydrate (65 mg, 259 μmol) was heated at 80° C. for 16 h. The reaction mixture was quenched with water. The aqeuous layer was extracted with CH₂Cl₂, dried (Na₂SO₄) and concentrated under reduced pressure. The crude residue was purified by prep-HPLC to give (S,E)-11-methyl-1⁷-(1-(oxetan-3-yl)-1H-1,2,3-triazol-4-yl)-1²,1³-dihydro-1¹H-2,6-diaza-1(2,1)-benzo[d]imidazola-4(1,3)-benzenacycloundecaphane-3,5-dione (60 mg, 23%).

¹H NMR (DMSO-d₆) @ 353.2 K δ: 8.83 (bs, 1H), 8.64 (s, 1H), 8.11 (d, 1H), 7.75 (d, 1H), 7.64 (d, 1H), 7.57 (m, 1H), 7.46 (m, 1H), 7.26 (m, 1H), 7.14 (m, 1H), 5.95 (m, 1H), 5.13 (m, 2H), 5.00, m2H), 4.49 (bs, 1H), 3.53 (bs, 1H), 3.03 (bs, 1H), 1.74 (m, 2H), 1.47 (d, 3H); LC/MS: M+H=486.30

### Example 130: (S,E)-11-methyl-1⁴-(3-methyl-2-oxoimidazolidin-1-yl)-1²,1³-dihydro-1¹H-2,6-diaza-1(2,3)-imidazo[4,5-c]pyridina-4(1,3)-benzenacycloundecaphane-3,5-dione

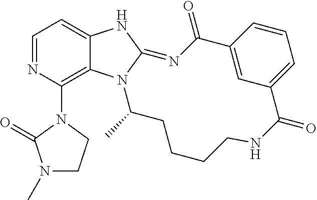 [SMILES: C[C@H]1CCCCNC(=O)c2cccc(c2)C(=O)/N=C2\Nc3ccnc(N4CCN(C)C4=O)c3N21]

Example 130 was prepared from (S,E)-1⁴-chloro-11-methyl-1²,1³-dihydro-1¹H-2,6-diaza-1(2,3)-imidazo[4,5-c]pyridina-4(1,3)-benzenacycloundecaphane-3,5-dione and 1-methylimidazolidin-2-one analogous to the preparation of Example 126.

¹H NMR (DMSO-d₆) @ 353.1 K δ: 8.85 (bs, 1H), 8.18 (d, 1H), 8.11 (d, 1H), 7.76 (d, 1H), 7.59 (m, 1H), 7.51 (m, 1H), 7.42 (d, 1H), 5.15 (m, 1H), 3.09 (m, 1H), 2.82 (s, 3H), 1.93 (m, 3H), 1.66 (m, 3H), 1.55 (m, 1H), 1.44 (m, 1H); LC/MS: M+H=462.35.

### Example 131: (E)-N,N-dimethyl-3′,5′-dioxospiro[cyclopropane-1,11′-2,6-diaza-1(2,1)-benzo[d]imidazola-4(1,3)-benzenacycloundecaphane]-7′-carboxamide

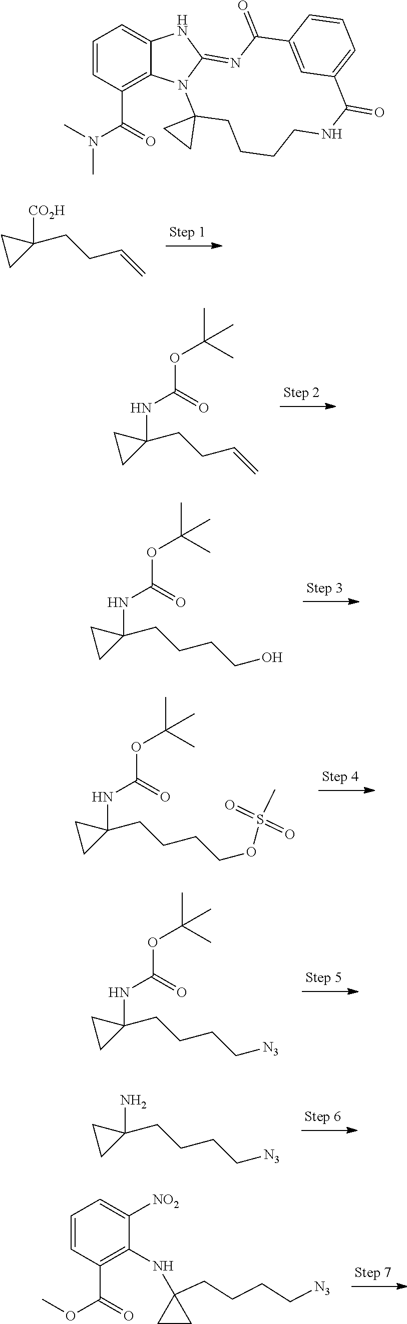

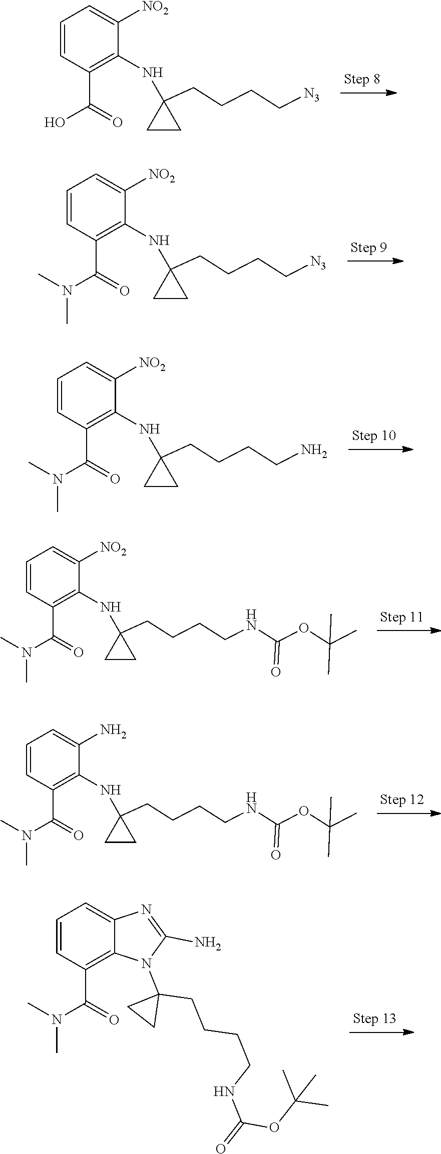

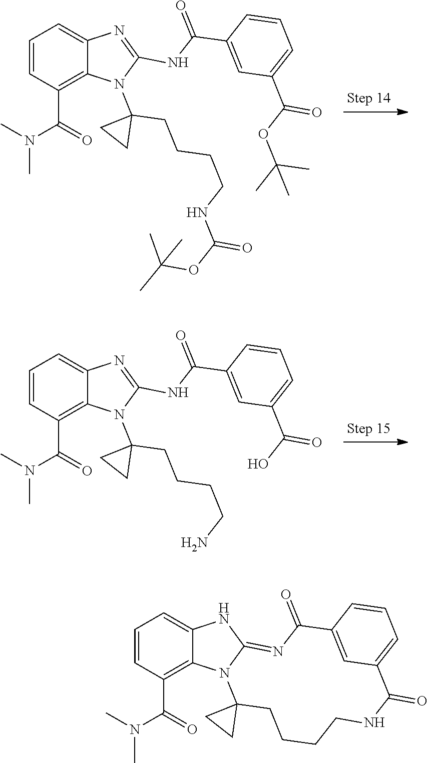 [SMILES: CN(C)C(=O)c1cccc2nc(NC(=O)c3cccc(C(=O)OC(C)(C)C)c3)n(C3(CCCCNC(=O)OC(C)(C)C)CC3)c12 ; CN(C)C(=O)c1cccc2nc(NC(=O)c3cccc(C(=O)O)c3)n(C3(CCCCN)CC3)c12 ; CN(C)C(=O)c1cccc2c1N1/C(=N/C(=O)c3cccc(c3)C(=O)NCCCCC13CC3)N2]

### Step 1: Preparation of tert-butyl (1-(but-3-en-1-yl)cyclopropyl)carbamate

To a stirred solution of 1-(but-3-en-1-yl)cyclopropane-1-carboxylic acid (4.6 g, 32.8 mmol) in tert-butanol (55.2 ml) and TEA (13.7 ml, 98.4 mmol) was added diphenylphosphonic azide (10.6 ml, 49.2 mmol). The reaction mixture was stirred at 120° C. for 16 h. The reaction mixture was quenched with water and extracted with CH₂Cl₂. The organic layer was dried with Na₂SO₄ and concentrated under reduced pressure. The crude product was purified by flash chromatography (SiO₂, 8% EtOAC/heptane v/v) to give tert-butyl (1-(but-3-en-1-yl)cyclopropyl)carbamate (2.6 g, 35%).

¹H NMR (DMSO-d₆) δ: 5.85 (m, 1H), 4.98 (d, 1H), 4.90 (d, 1H), 2.09 (m, 2H), 1.50 (m, 2H), 1.36 (s, 9H), 0.56 (m, 2H), 0.47 (m, 2H).

### Step 2: Preparation of tert-butyl (1-(4-hydroxybutyl)cyclopropyl)carbamate

To a stirred solution of tert-butyl (1-(but-3-en-1-yl)cyclopropyl)carbamate (8 g, 37.1 mmol) in THF (55.2 ml) was added 1M borane THF complex (6.38 g, 74.2 mmol) at 0° C. The reaction mixture was stirred at rt for 2 h. The reaction mixture was cooled to 0° C. and NaOH (10.4 g, 260 mmol) and 33% hydrogen peroxide (79.1 ml, 1.11 mol) were added. The resulting mixture was stirred at rt for 2 h. the reaction mixture was quenched with water and neutralized by 2N HCl and extracted with CH₂Cl₂. The organic layer was dried with Na₂SO₄ and concentrated under reduced pressure. The crude was purified by flash chromatography (SiO₂, 3% EtOAC/heptane v/v) to give tert-butyl (1-(4-hydroxybutyl)cyclopropyl)carbamate (2.0 g, 20%).

¹H NMR (DMSO-d₆) δ: 3.34 (m, 2H), 1.35 (m, 4H), 1.31 (s, 9H), 0.50 (bs, 2H), 0.43 (bs, 2H).

### Step 3: Preparation of 4-(1-((tert-butoxycarbonyl)amino)cyclopropyl)butyl methanesulfonate

To a stirred solution of tert-butyl (1-(4-hydroxybutyl)cyclopropyl)carbamate (2 g, 8.72 mmol) in CH₂Cl₂ (55.2 ml) was added TEA (3.65 ml, 26.2 mmol) and methanesulfonyl chloride (1.01 ml, 13.1 mmol) at 0° C. The reaction mixture was stirred at rt for 2 h. The reaction mixture was diluted with water, extracted with CH₂Cl₂. The organic phase was dried with Na₂SO₄ and concentrated under reduced pressure. The crude was purified by flash chromatography (SiO₂, 28% EtOAC/heptane v/v) to give 4-(1-((tert-butoxycarbonyl)amino)cyclopropyl)butyl methanesulfonate (3.0 g, 89%).

¹H NMR (DMSO-d₆) δ: 4.17 (m, 2H), 3.35 (s, 3H), 1.66 (m, 2H), 0.56 (m, 2H), 0.46 (m, 2H).

### Step 4: Preparation of tert-butyl (1-(4-azidobutyl)cyclopropyl)carbamate

To a stirred solution of 4-(1-((tert-butoxycarbonyl)amino)cyclopropyl)butyl methanesulfonate (3.4 g, 11.1 mmol) in DMF (56.72 ml) was added sodium azide (2.16 g, 33.2 mmol). The reaction mixture was heated at 60° C. for 16 h. The reaction mixture was diluted with water, extracted with EtOAc. The organic phase was dried with Na₂SO₄ and concentrated under reduced pressure. The crude was purified by flash chromatography (SiO₂, 5% EtOAC/heptane v/v) to give tert-butyl (1-(4-azidobutyl)cyclopropyl)carbamate (1.9 g, 62%).

¹H NMR (DMSO-d₆) δ: 1.57 (m, 2H), 1.36 (s, 9H), 0.56 (m, 2H), 0.43 (m, 2H).

### Step 5: Preparation of 1-(4-azidobutyl)cyclopropan-1-amine

To a stirred solution of tert-butyl (1-(4-azidobutyl)cyclopropyl)carbamate (1.9 g, 7.47 mmol) in CH₂Cl₂ (30 ml) was added TFA (5 ml). The reaction mixture stirred at rt for 2 h. The reaction mixture was concentrated under reduced pressure. The crude product was used without further purification.

### Step 6: Preparation of methyl 2-((1-(4-azidobutyl)cyclopropyl)amino)-3-nitrobenzoate

To a stirred solution of methyl 2-fluoro-3-nitrobenzoate (1.9 g, 9.54 mmol) and 1-(4-azidobutyl)cyclopropan-1-amine (1.6 g, 10.5 mmol) in DMF (19 ml) was added N,N-diisopropylethylamine (6.65 ml, 38.2 mmol). The reaction mixture stirred at rt for 16 h. The reaction mixture was quenched with water and extracted with EtOAc. The organic layer was dried with Na₂SO₄ and concentrated under reduced pressure. The crude was purified by flash chromatography (SiO₂, 8% EtOAC/heptane v/v) to give methyl 2-((1-(4-azidobutyl)cyclopropyl)amino)-3-nitrobenzoate (1.2 g, 37%).

¹H NMR (DMSO-d₆) δ: 8.42 (s, 1H), 8.03 (d, 1H), 7.92 (d, 1H), 6.84 (m, 1H), 3.91 (s, 3H), 3.31 (m, 2H), 1.47 (m, 6H), 0.71 (m, 2H), 0.59 (m, 2H); LC/MS: M+H=333.95.

### Step 7: Preparation of 2-((1-(4-azidobutyl)cyclopropyl)amino)-3-nitrobenzoic acid

To a stirred solution of methyl 2-((1-(4-azidobutyl)cyclopropyl)amino)-3-nitrobenzoate (2.0 g, 6 mmol) in THF (15 ml) and water (5 ml) was added LiOH (1.26 g, 30 mmol). The reaction mixture stirred at rt for 16 h. The reaction mixture was concentrated under reduced pressure and the residue was diluted with water, cooled to 0° C. and acidified (pH 4) with 1 M HCl. The formed precipitate was collected by filtration, was washed with cold water and dried under vacuum to give 2-((1-(4-azidobutyl)cyclopropyl)amino)-3-nitrobenzoic acid (1.8 g, 92%).

¹H NMR (DMSO-d₆) δ: 8.77 (s, 1H), 8.06 (d, 1H), 7.84 (d, 1H), 6.81 (m, 1H), 3.28 (m, 2H), 1.49 (m, 6H), 0.67 (m, 2H), 0.58 (m, 2H); LC/MS: M+H=320.15.

### Step 8: Preparation of 2-((1-(4-azidobutyl)cyclopropyl)amino)-N,N-dimethyl-3-nitrobenzamide

To a stirred solution of 2-((1-(4-azidobutyl)cyclopropyl)amino)-3-nitrobenzoic acid (2.0 g, 6.26 mmol) in MeCN (201 ml), was added TCFH (2.55 g, 18.8 mmol) and 1-methyl-1H-imidazole (2.5 ml, 31.3 mmol) followed by 2.0 M dimethyl amine (847 mg, 18.8 mmol). The reaction mixture stirred at rt for 16 h. The reaction mixture was diluted with water, and extracted with EtOAc. The organic layer was dried with Na₂SO₄ and concentrated under reduced pressure. The crude was purified by flash chromatography (SiO₂, 45% EtOAC/heptane v/v) to give 2-((1-(4-azidobutyl)cyclopropyl)amino)-N,N-dimethyl-3-nitrobenzamide (1.8 g, 73%).

¹H NMR (DMSO-d₆) δ: 8.07 (m, 1H), 7.78 (s, 1H), 7.40 (m, 1H), 6.91 (m, 1H), 3.24 (m, 2H), 2.99 (s, 3H), 2.80 (s, 3H), 1.45 (m, 4H), 1.27 (m, 2H), 0.76 (m, 2H), 0.56 (m, 2H); LC/MS: M+H=347.85.

### Step 9: Preparation of 2-((1-(4-aminobutyl)cyclopropyl)amino)-N,N-dimethyl-3-nitrobenzamide

PPh3 (2.73 g, 10.4 mmol) was added to a solution of 2-((1-(4-azidobutyl)cyclopropyl)amino)-N,N-dimethyl-3-nitrobenzamide (1.8 g, 5.2 mmol) in THF (15 ml) and water (5 ml). The reaction mixture stirred at rt for 16 h. The reaction mixture was concentrated under reduced pressure and the residue was diluted with water (15 ml), cooled to 0° C. and acidified (pH 4) with 1 M HCl. The aqueous phase was washed with EtOAc, and basified with NH₄OH. The aqueous phase was extracted with EtOAc and the organic layer was dried with Na₂SO₄ and concentrated under reduced pressure. The product was used without further purification.

¹H NMR (DMSO-d₆) δ: 8.06 (d, 1H), 7.76 (s, 1H), 7.40 (d, 1H), 6.90 (m, 1H), 2.98 (s, 3H), 2.79 (s, 3H), 1.45 (m, 2H), 1.22 (m, 4H), 0.76 (m, 2H), 0.55 (m, 2H); LC/MS: M+H=321.10.

### Step 10: Preparation of tert-butyl (4-(1-((2-(dimethylcarbamoyl)-6-nitrophenyl)amino)cyclopropyl)butyl)carbamate

N,N-diisopropylethylamine (2.42 ml, 14 mmol) and di-tert-butyl dicarbonate (1.29 ml, 5.62 mmol) were added to a solution of 2-((1-(4-aminobutyl)cyclopropyl)amino)-N,N-dimethyl-3-nitrobenzamide (1.5 g, 4.68 mmol) in CH₂Cl₂ (20 ml). The reaction mixture stirred at 0° C. overnight. The reaction mixture was diluted with water and extracted with CH₂Cl₂. The organic layer was dried with Na₂SO₄ and concentrated under reduced pressure. The crude was purified by flash chromatography (SiO₂) to give tert-butyl (4-(1-((2-(dimethylcarbamoyl)-6-nitrophenyl)amino)cyclopropyl)butyl)carbamate (2.0 g, 98%).

¹H NMR (DMSO-d₆) δ: 8.06 (d, 1H), 7.77 (s, 1H), 7.40 (d, 1H), 6.90 (m, 1H), 6.70 (m, 1H), 2.98 (s, 3H), 2.79 (s, 3H), 1.45 (m, 2H), 1.24 (m, 4H), 0.74 (m, 2H), 0.55 (m, 2H); LC/MS: M+H=421.25.

### Step 11: Preparation of tert-butyl (4-(1-((2-amino-6-(dimethylcarbamoyl)phenyl)amino)cyclopropyl)butyl)carbamate

Pd/C (202 mg, 1.9 mmol) was added to a solution of tert-butyl (4-(1-((2-(dimethylcarbamoyl)-6-nitrophenyl)amino)cyclopropyl)butyl)carbamate (0.4 g, 951 μmol) in MeOH (20 ml). The reaction was hydrogenated under a hydrogen atmosphere at rt for 1 h. The reaction mixture was filtered through celite and the filtrate was concentrated under reduced pressure. The crude product was used without further purification.

LC/MS: M+H=391.10.

### Step 12: Preparation of tert-butyl (4-(1-(2-amino-7-(dimethylcarbamoyl)-1H-benzo[d]imidazol-1-yl)cyclopropyl)butyl)carbamate

Cyanogen bromide (68 mg, 639 μmol) was added to a solution of tert-butyl (4-(1-((2-amino-6-(dimethylcarbamoyl)phenyl)amino)cyclopropyl)butyl)carbamate (0.4 g, 533 μmol) in MeOH (10.4 ml), MeCN (2.6 ml) and water (2.6 ml). The reaction mixture was stirred at 55° C. for 3 h. The reaction mixture was diluted with water, extracted with EtOAc and concentrated under reduced pressure. The crude residue was purified by flash chromatography (SiO2, 10% MeOH in CH₂Cl₂) to give tert-butyl (4-(1-(2-amino-7-(dimethylcarbamoyl)-1H-benzo[d]imidazol-1-yl)cyclopropyl)butyl)carbamate (200 mg, 62%).

¹H NMR (DMSO-d₆) δ: 7.11 (d, 1H), 6.94 (m, 1H), 6.65 (d, 1H), 6.28 (bs, 2H), 3.06 (s, 3H), 2.89 (s, 3H), 1.06 (m, 2H), 0.75 (m, 2H); LC/MS: M+H=416.15.

### Step 13: Preparation of tert-butyl 3-((1-(1-(4-((tert-butoxycarbonyl)amino)butyl)cyclopropyl)-7-(dimethylcarbamoyl)-1H-benzo[d]imidazol-2-yl)carbamoyl)benzoate

To a solution of tert-butyl (4-(1-(2-amino-7-(dimethylcarbamoyl)-1H-benzo[d]imidazol-1-yl)cyclopropyl)butyl)carbamate (0.4 g, 533 μmol) and 3-(tert-butoxycarbonyl)benzoic acid (185 mg, 830 μmol) in MeCN (40.4 ml) was added TCFH (388 mg, 1.38 mmol) at 0° C. followed by 1-methyl-1H-imidazole (185 μl, 2.77 mmol). The reaction was stirred at rt for 1 h. After completion, the reaction mixture was quenched with water and extracted with EtOAc and the organic phase was dried with Na₂SO₄ and concentrated under reduced pressure. The product was purified with flash chromatography (SiO2, 45% EtOAc/Heptane) to give tert-butyl 3-((1-(1-(4-((tert-butoxycarbonyl)amino)butyl)cyclopropyl)-7-(dimethylcarbamoyl)-1H-benzo[d]imidazol-2-yl)carbamoyl)benzoate (180 mg, 49%).

¹H NMR (DMSO-d₆) δ: 8.89 (s, 1H), 8.43 (d, 1H), 8.04 (d, 1H), 7.60 (m, 2H), 7.24 (m, 1H), 7.07 (d, 1H), 3.08 (s, 3H), 2.92 (s, 3H), 1.59 (s, 9H), 1.30 (s, 9H); LC/MS: M+H=620.50.

### Step 14: Preparation of 3-((1-(1-(4-aminobutyl)cyclopropyl)-7-(dimethylcarbamoyl)-1H-benzo[d]imidazol-2-yl)carbamoyl)benzoic acid

TFA (310 μl) was added to a solution of tert-butyl 3-((1-(1-(4-((tert-butoxycarbonyl)amino)butyl)cyclopropyl)-7-(dimethylcarbamoyl)-1H-benzo[d]imidazol-2-yl)carbamoyl)benzoate (250 mg, 403 μmol) in CH₂Cl₂ (5 ml) at 0° C. The reaction was stirred at rt for 16 h. The crude reaction mixture concentrated under reduced pressure and the product was used without further purification.

¹H NMR (DMSO-d₆) δ: 8.87 (s, 1H), 8.47 (d, 1H), 8.09 (d, 1H), 7.62 (m, 2H), 7.25 (m, 1H), 7.07 (d, 1H), 3.08 (s, 3H), 2.92 (s, 3H), 2.67 (m, 2H), 1.49 (m, 2H), 1.45 (m, 4H); LC/MS: M+H=464.25.

### Step 15: Preparation of (E)-N,N-dimethyl-3′,5′-dioxospiro[cyclopropane-1,11′-2,6-diaza-1(2,1)-benzo[d]imidazola-4(1,3)-benzenacycloundecaphane]-7′-carboxamide

To a solution of 1-methyl-1H-imidazole (86 μl, 1.08 mmol) and TCFH (121 mg, 431 mol) in MeCN (700 ml) was added a solution of 3-((1-(1-(4-aminobutyl)cyclopropyl)-7-(dimethylcarbamoyl)-1H-benzo[d]imidazol-2-yl)carbamoyl)benzoic acid (100 mg, 216 μmol) in DMF (5 ml) at rt. The reaction mixture was stirred at rt for 16 h. The reaction mixture was quenched with water and concentrated under reduced pressure. The crude product was triturated with water and the precipitate was collected by filtration. The product was purified by reverse phase HPLC to give (E)-N,N-dimethyl-3′,5′-dioxospiro[cyclopropane-1,11′-2,6-diaza-1(2,1)-benzo[d]imidazola-4(1,3)-benzenacycloundecaphane]-7′-carboxamide (18 mg, 18%).

¹H NMR (DMSO-d₆) δ: 9.15 (bs, 1H), 8.11 (d, 1H), 7.90 (m, 1H), 7.74 (d, 1H), 7.57 (m, 2H), 7.22 (m, 1H), 7.03 (d, 1H), 3.06 (s, 3H), 2.97 (s, 3H), 1.78 (m, 2H), 1.41 (m, 1H), 1.38 (m, 2H), 1.00 (m, 1H), 0.81 (m, 1H), 0.75 (m, 1H); LC/MS: M+H=446.25.

### Examples 132 and 133: (E)-11-methyl-1⁷-(5-methyl-1,3,4-oxadiazol-2-yl)-1²,1³-dihydro-1¹H-2,6-diaza-1(2,1)-benzo[d]imidazola-4(1,3)-benzenacycloundecaphane-3,5-dione

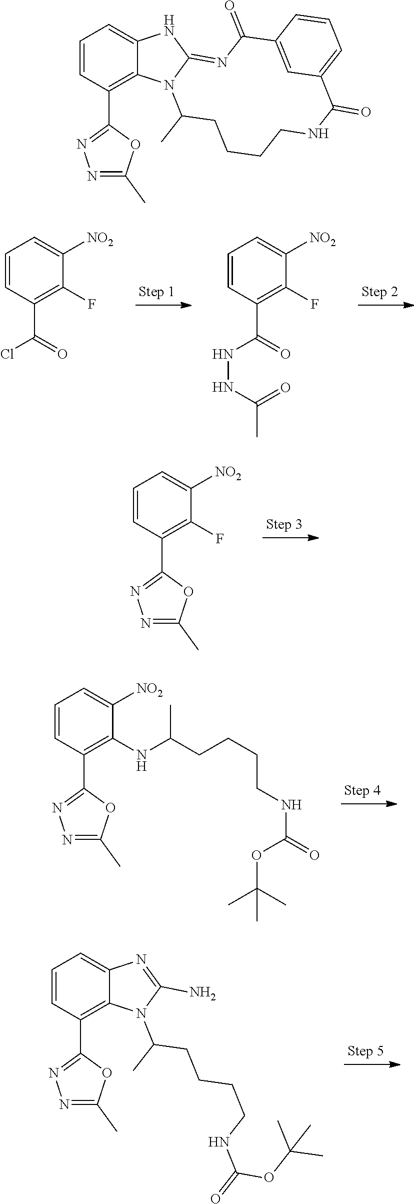 [SMILES: Cc1nnc(-c2cccc3c2N2/C(=N/C(=O)c4cccc(c4)C(=O)NCCCCC2C)N3)o1 ; O=C(Cl)c1cccc([N+](=O)[O-])c1F ; CC(=O)NNC(=O)c1cccc([N+](=O)[O-])c1F ; Cc1nnc(-c2cccc([N+](=O)[O-])c2F)o1 ; Cc1nnc(-c2cccc([N+](=O)[O-])c2NC(C)CCCCNC(=O)OC(C)(C)C)o1 ; Cc1nnc(-c2cccc3nc(N)n(C(C)CCCCNC(=O)OC(C)(C)C)c23)o1]

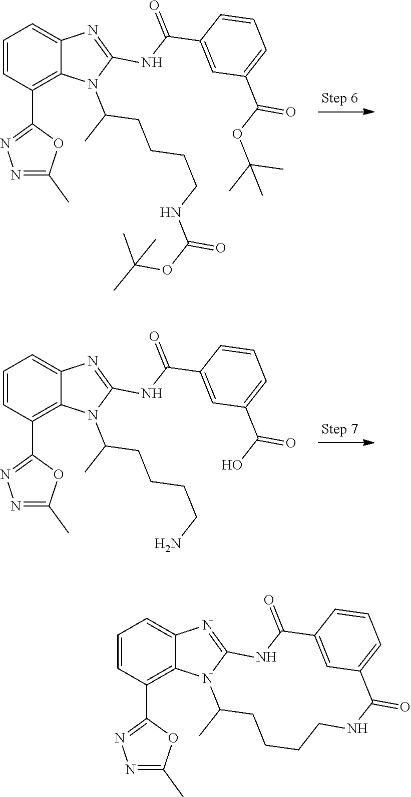 [SMILES: Cc1nnc(-c2cccc3nc(NC(=O)c4cccc(C(=O)OC(C)(C)C)c4)n(C(C)CCCCNC(=O)OC(C)(C)C)c23)o1 ; Cc1nnc(-c2cccc3nc(NC(=O)c4cccc(C(=O)O)c4)n(C(C)CCCCN)c23)o1 ; Cc1nnc(-c2cccc3nc4n(c23)C(C)CCCCNC(=O)c2cccc(c2)C(=O)N4)o1]

### Step 1: Preparation of N′-acetyl-2-fluoro-3-nitrobenzohydrazide

A solution of acetohydrazide (91 mg, 1.23 mmol) with a few drops DMF in MeCN (0.5 ml) and N,N-diisopropylethylamine (212 μl, 1.23 mmol) were added to a solution of 2-fluoro-3-nitrobenzoyl chloride (250 mg, 1.23 mmol) in MeCN (10 ml) at rt. The reaction mixture was stirred at rt for 1 h. The reaction mixture was concentrated under reduced pressure, diluted with EtOAc and NaHCO₃(aq). The phases were separated, and the aqueous phase was acidified to pH2-3 and extracted with EtOAc. The organic phase was washed with brine, dried and concentrated to dryness. The product (237 mg, 80%) was used without further purification.

¹H NMR (DMSO-d₆) δ: 8.30 (m, 1H), 7.91 (m, 1H), 7.33 (d, 1H), 1.93 (s, 3H).

### Step 2: Preparation of 2-(2-fluoro-3-nitrophenyl)-5-methyl-1,3,4-oxadiazole

Phosphoryl chloride (116 μl, 425 μmol) was added to a solution of ofN′-acetyl-2-fluoro-3-nitrobenzohydrazide (100 mg, 415 μmol) in toluene (5.0 ml) at rt. The suspension was heated at 120° C. in a sealed vial for 30 min. The mixture was cooled to rt, diluted with EtOAc, washed with NaHCO₃ and brine. The organic phase was dried and concentrated. The product (83 mg, 89%) was used without further purification.

¹H NMR (DMSO-d₆) δ: 8.32 (m, 2H), 7.60 (m, 1H), 2.58 (s, 3H).

### Step 3: Preparation of tert-butyl (5-((2-(5-methyl-1,3,4-oxadiazol-2-yl)-6-nitrophenyl)amino)hexyl)carbamate

tert-Butyl (5-aminohexyl)carbamate (99.8 mg, 462 μmol) was added to a mixture of 2-(2-fluoro-3-nitrophenyl)-5-methyl-1,3,4-oxadiazole (103 mg, 462 μmol) and K₂CO₃ (128 mg, 923 μmol) in MeCN (10 ml). The reaction mixture was stirred at RT for 28 h. The reaction mixture was filtered and then the filtrate was evaporated to remove the volatiles. The crude was dissolved in EtOAc (10 ml), washed with 10% AcOH followed by NaHCO₃(aq) and brine. The organic phase was dried and concentrated. The crude product (135 mg) was used without further purification.

¹H NMR (chloroform-d) δ: 8.06 (d, 1H), 7.92 (d, 1H), 7.81 (d, 1H), 6.80 (m, 1H), 3.76 (m, 1H), 3.02 (m, 2H), 1.43 (s, 9H), 1.21 (d, 3H).

### Step 4: Preparation of tert-butyl (5-(2-amino-7-(5-methyl-1,3,4-oxadiazol-2-yl)-1H-benzo[d]imidazol-1-yl)hexyl)carbamate

A solution of tert-butyl (5-((2-(5-methyl-1,3,4-oxadiazol-2-yl)-6-nitrophenyl)amino)hexyl)carbamate (135 mg, 322 μmol) in MeOH (10 ml) was hydrogenated over 10% Pd/C (15 mg, 14.5 μmol) at rt for 2 h. The reaction mixture was filtered through celite into water (2.0 ml) and cyanogen bromide (41 mg, 386 μmol). The resulting solution was stirred at RT for 17 h and then heated at 40° C. for one day. The reaction mixture was concentrated under reduced pressure and purified by flash chromatography (silica gel, 0.5% MeOH in CH₂Cl₂ with 1% NH₃ (28% aq.)) to give tert-butyl (5-(2-amino-7-(5-methyl-1,3,4-oxadiazol-2-yl)-1H-benzo[d]imidazol-1-yl)hexyl)carbamate (71 mg, 55%).

¹H NMR (chloroform-d) δ: 7.51 (m, 1H), 7.39 (m, 1H), 7.20 (m, 1H), 5.15 (m, 1H), 4.70 (m, 1H), 3.01 (m, 2H), 2.71 (s, 3H).

### Step 5: Preparation of tert-butyl 3-((1-(6-((tert-butoxycarbonyl)amino)hexan-2-yl)-7-(5-methyl-1,3,4-oxadiazol-2-yl)-1H-benzo[d]imidazol-2-yl)carbamoyl)benzoate

N,N-diisopropylethylamine (78 μl, 454 μmol) was added to a solution of 3-(tert-butoxycarbonyl)benzoic acid (135 mg, 322 μmol) and HATU (86 mg, 227 μmol) in MeCN (3 ml). The mixture was stirred for 2 min and then a solution of tert-butyl (5-(2-amino-7-(dimethylcarbamoyl)-1H-benzo[d]imidazol-1-yl)hexyl)carbamate (70 mg, 175 μmol) in MeCN (8 ml) was added. The reaction mixture was stirred at rt overnight and then evaporated to dryness under reduced pressure. The crude product was purified twice by flash chromatography (SiO₂, 0 to 10% MeOH in CH₂Cl₂ and then 0 to 50% EtOAc in pet.-ether) to give tert-butyl 3-((1-(6-((tert-butoxycarbonyl)amino)hexan-2-yl)-7-(5-methyl-1,3,4-oxadiazol-2-yl)-1H-benzo[d]imidazol-2-yl)carbamoyl)benzoate (71 mg, 67%).

¹H NMR (chloroform-d) δ: 8.93 (s, 1H), 8.44 (d, 1H), 8.15 (d, 1H), 7.53 (m, 3H), 7.34 (m, 1H), 4.77-4.55 (m, 2H), 3.06-2.91 (m, 2H), 2.70 (s, 4H), 2.03-1.91 (m, 1H), 1.79 (d, 3H), 1.63 (s, 1¹H), 1.38 (s, 14H).

### Step 6: Preparation of 3-((1-(6-aminohexan-2-yl)-7-(5-methyl-1,3,4-oxadiazol-2-yl)-1H-benzo[d]imidazol-2-yl)carbamoyl)benzoic acid

4M HCl in dioxane (12 ml) was added to tert-butyl 3-((1-(6-((tert-butoxycarbonyl)amino)hexan-2-yl)-7-(5-methyl-1,3,4-oxadiazol-2-yl)-1H-benzo[d]imidazol-2-yl)carbamoyl)benzoate (71 mg, 117 μmol). The reaction mixture was stirred at rt under argon for 4 h and then concentrated to dryness under reduced pressure. The crude product was used without further purification.

### Step 7: Preparation of (E)-11-methyl-1⁷-(5-methyl-1,3,4-oxadiazol-2-yl)-1²,1³-dihydro-1¹H-2,6-diaza-1(2,1)-benzo[d]imidazola-4(1,3)-benzenacycloundecaphane-3,5-dione

A mixture of N,N-diisopropylethylamine (101 μl, 584 μmol) and (54 mg, 117 μmol) in THF (232 ml) was sonicated to give a clear solution. HATU (67 mg, 175 μmol) was added, and the reaction mixture was stirred overnight at rt. The reaction mixture was concentrated to dryness under reduced pressure and the crude was purified using reverse phase chromatography (C18 column, 20-50% MeCN in water containing 0.1% TFA) to give (E)-11-methyl-1⁷-(5-methyl-1,3,4-oxadiazol-2-yl)-1²,1³-dihydro-1¹H-2,6-diaza-1(2,1)-benzo[d]imidazola-4(1,3)-benzenacycloundecaphane-3,5-dione (8 mg, 13%). The enantiomers were separated using chiral chromatography.

### Example 132, Enantiomer 1

¹H NMR (chloroform-d) δ: 12.05 (s, 1H), 8.99 (s, 1H), 8.27 (d, 1H), 7.96 (d, 1H), 7.60 (m, 1H), 7.53 (m, 2H), 7.34 (m, 1H), 5.88 (s, 1H), 5.15 (s, 1H), 3.99-3.66 (m, 1H), 3.41-2.95 (m, 2H), 2.70 (s, 3H), 2.15-1.86 (m, 3H), 1.76-1.45 (m, 8H), 1.38-1.07 (m, 4H); LC/MS: M+H=445.0.

Daicel IA column (0.46×25 cm), 3% EtOH in CH₂Cl₂, Flow rate: 0.6 ml/min, Rt=12 min

### Example 133, Enantiomer 2

¹H NMR (chloroform-d) δ: 8.27 (d, 1H), 7.96 (d, 1H), 7.60 (m, 1H), 7.53 (m, 2H), 7.32 (m, 1H), 5.91 (s, 1H), 5.26-5.00 (m, 1H), 3.99-3.67 (m, 1H), 3.37-3.04 (m, 1H), 2.69 (s, 3H), 2.14-1.86 (m, 3H), 1.74-1.42 (m, 6H), 1.38-1.09 (m, 4H);

LC/MS: M+H=445.0.

Daicel IA column (0.46×25 cm), 3% EtOH in CH₂Cl₂, Flow rate: 0.6 ml/min, Rt=15 min

### Example 134: (S,E)-1⁷-(4-isopropyl-5-methyl-4H-1,2,4-triazol-3-yl)-11-methyl-1²,1³-dihydro-1¹H-2,6-diaza-1(2,1)-benzo[d]imidazola-4(1,3)-benzenacycloundecaphane-3,5-dione

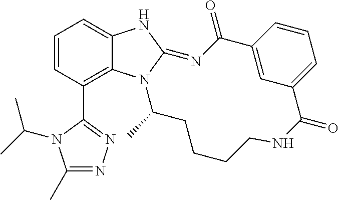 [SMILES: Cc1nnc(-c2cccc3c2N2/C(=N/C(=O)c4cccc(c4)C(=O)NCCCC[C@@H]2C)N3)n1C(C)C]

Example 134 was prepared from tert-butyl (S)-(5-((2-(5-methyl-1,3,4-oxadiazol-2-yl)-6-nitrophenyl)amino)hexyl)carbamate and propan-2-amine analogous to the preparation of Examples 57 and 96.

¹H NMR (DMSO-d₆) δ: 12.88 (s, 1H), 8.72 (s, 1H), 8.11 (d, 1H), 7.84-7.67 (m, 3H), 7.59 (m, 1H), 7.35 (m, 1H), 7.24 (d, 1H), 4.34 (s, 1H), 3.76-3.64 (m, 2H), 2.94 (s, 2H), 2.65 (s, 3H), 2.00-1.08 (m, 14H); LC/MS: M+H=486.3.

### Examples 135 and 136: (E)-11-methyl-1⁷-(1-methyl-1H-imidazol-2-yl)-1²,1³-dihydro-1¹H-2,6-diaza-1(2,1)-benzo[d]imidazola-4(1,3)-benzenacycloundecaphane-3,5-dione

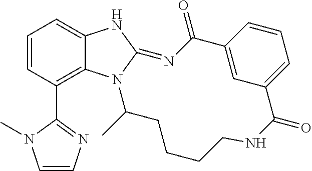 [SMILES: CC1CCCCNC(=O)c2cccc(c2)C(=O)/N=C2\Nc3cccc(-c4nccn4C)c3N21]

Examples 135 and 136 were prepared from 2-(2-chloro-3-nitrophenyl)-1H-imidazole and methyl iodide analogous to the preparation of Examples 90 and 91. The enantiomers of (E)-11-methyl-1⁷-(1-methyl-1H-imidazol-2-yl)-1²,1³-dihydro-1¹H-2,6-diaza-1(2,1)-benzo[d]imidazola-4(1,3)-benzenacycloundecaphane-3,5-dione were separated using chiral chromatography.

### Example 135, Enantiomer 1

¹H NMR (DMSO-d₆) @ 352.5 K δ: 8.81 (bs, 1H), 8.08 (d, 1H), 7.65 (d, 1H), 7.61 (d, 1H), 7.53 (m, 1H), 7.37 (m, 1H), 7.30 (s, 1H), 7.12 (m, 1H), 7.05 (s, 1H), 6.97 (m, 1H), 3.45 (s, 3H); LC/MS: M+H=443.37.

Chiralpak-IG (4.6×250) mm 5 um MeOH/EtOH 50:50 (v/v), Flow Rate: 1.0 mL/min, Rt=10.45 min

### Example 136, Enantiomer 2

¹H NMR (DMSO-d₆) @ 353.0 K δ: 8.80 (bs, 1H), 8.07 (d, 1H), 7.65 (d, 1H), 7.61 (d, 1H), 7.53 (m, 1H), 7.37 (m, 1H), 7.30 (s, 1H), 7.12 (m, 1H), 7.04 (s, 1H), 6.93 (m, 1H), 3.45 (s, 3H); LC/MS: M+H=443.37.

Chiralpak-IG (4.6×250) mm 5 um MeOH/EtOH 50:50 (v/v), Flow Rate: 1.0 mL/min, Rt=13.14 min

### Examples 137 and 138: (E)-1⁷-(1-(difluoromethyl)-1H-1,2,4-triazol-3-yl)-11-methyl-1²,1³-dihydro-1¹H-2,6-diaza-1(2,1)-benzo[d]imidazola-4(1,3)-benzenacycloundecaphane-3,5-dione

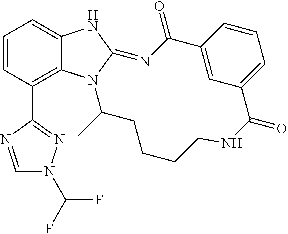 [SMILES: CC1CCCCNC(=O)c2cccc(c2)C(=O)/N=C2\Nc3cccc(-c4ncn(C(F)F)n4)c3N21]

Examples 137 and 138 were prepared from 3-(2-chloro-3-nitrophenyl)-1-(difluoromethyl)-1H-1,2,4-triazole analogous to the preparation of Examples 83 and 84. Briefly, to a solution of 5-(2-chloro-3-nitrophenyl)-1H-1,2,4-triazole (4.0 g, 17.6 mmol) in MeCN (40.6 ml) was added potassium fluoride (2.05 g, 35.3 mmol) and diethyl (bromodifluoromethyl)phosphonate (5.18 g, 19.4 mmol). The reaction was stirred at rt for 16 h. The reaction was quenched with water and extracted with CH₂Cl₂. The organic phase was concentrated under reduced pressure. The residue was purified by prep-HPLC to give 5-(2-chloro-3-nitrophenyl)-1-(difluoromethyl)-1H-1,2,4-triazole (150 mg, 3%) and 3-(2-chloro-3-nitrophenyl)-1-(difluoromethyl)-1H-1,2,4-triazole (450 mg, 9%).

### 5-(2-chloro-3-nitrophenyl)-1-(difluoromethyl)-1H-1,2,4-triazole

¹H NMR (DMSO-d₆) δ: 8.53 (s, 1H), 8.34 (d, 1H), 7.99 (d, 1H), 7.97 (m, 1H), 7.82 (m, 1H); LC/MS: M+H=274.97.

### 3-(2-chloro-3-nitrophenyl)-1-(difluoromethyl)-1H-1,2,4-triazole

¹H NMR (DMSO-d₆) δ: 9.31 (s, 1H), 8.18 (d, 2H), 8.17 (m, 1H), 7.75 (m, 1H);

LC/MS: M+H=274.97.

The enantiomers of (E)-1⁷-(1-(difluoromethyl)-1H-1,2,4-triazol-3-yl)-11-methyl-1²,1³-dihydro-1¹H-2,6-diaza-1(2,1)-benzo[d]imidazola-4(1,3)-benzenacycloundecaphane-3,5-dione were separated using chiral chromatography.

### Example 137, Enantiomer 1

¹H NMR (DMSO-d₆) δ: 9.29 (s, 1H), 8.77 (bs, 1H), 8.11 (d, 1H), 8.08 (m, 1H), 7.74 (m, 2H), 7.68 (m, 1H), 7.59 (m, 1H), 7.38 (m, 1H), 7.30 (m, 1H), 4.55 (m, 1H), 3.53 (m, 1H), 3.02 (m, 1H), 1.79 (m, 3H); LC/MS: M+H=480.13.

Chiralpak-IG (4.6×250) mm 5 um, 0.1% ammonium hydroxide in iPrOH/EtOAc 80:20 (v/v), Flow Rate: 1.0 mL/min, Rt=7.06 min

### Example 138, Enantiomer 2

¹H NMR (DMSO-d₆) δ: 9.27 (s, 1H), 8.76 (bs, 1H), 8.12 (d, 1H), 8.10 (m, 1H), 7.70 (m, 3H), 7.58 (m, 1H), 7.32 (m, 1H), 7.26 (m, 1H), 4.49 (m, 1H), 3.53 (m, 1H), 3.02 (m, 1H), 1.79 (m, 3H); LC/MS: M+H=480.14.

Chiralpak-IG (4.6×250) mm 5 um, 0.1% ammonium hydroxide in iPrOH/EtOAc 80:20 (v/v), Flow Rate: 1.0 mL/min, Rt=7.69

### Examples 139 and 140: (E)-1⁷-(1-(difluoromethyl)-1H-1,2,4-triazol-5-yl)-11-methyl-1²,1³-dihydro-1¹H-2,6-diaza-1(2,1)-benzo[d]imidazola-4(1,3)-benzenacycloundecaphane-3,5-dione

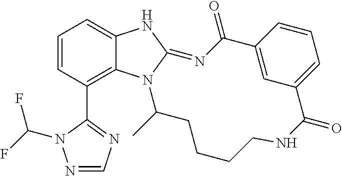 [SMILES: CC1CCCCNC(=O)c2cccc(c2)C(=O)/N=C2\Nc3cccc(-c4ncnn4C(F)F)c3N21]

Examples 139 and 140 were prepared from 5-(2-chloro-3-nitrophenyl)-1-(difluoromethyl)-1H-1,2,4-triazole analogous to the preparation of Examples 85 and 86.

The enantiomers of (E)-1⁷-(1-(difluoromethyl)-1H-1,2,4-triazol-5-yl)-11-methyl-1²,1³-dihydro-1¹H-2,6-diaza-1(2,1)-benzo[d]imidazola-4(1,3)-benzenacycloundecaphane-3,5-dione were separated using chiral chromatography.

### Example 139, Enantiomer 1

¹H NMR (DMSO-d₆) @ 353.2 K δ: 8.82 (s, 1H), 8.47 (s, 1H), 8.11 (d, 1H), 7.78 (m, 3H), 7.60 (m, 1H), 7.36 (m, 1H), 7.20 (d, 1H), 3.45 (m, 1H), 1.86 (m, 1H), 1.76 (m, 1H), 1.66 (m, 1H), 1.59 (m, 1H), 1.38 (m, 3H); LC/MS: M+H=480.14.

Chiralpak-IG (4.6×250) mm 5 um, 3% EtOH in CH₂Cl₂ (v/v), Flow Rate: 1.0 mL/min, Rt=11.52 min

### Example 140, Enantiomer 2

¹H NMR (DMSO-d₆) @ 352.2 K δ: 8.82 (s, 1H), 8.47 (s, 1H), 8.11 (d, 1H), 7.78 (m, 3H), 7.60 (m, 1H), 7.36 (m, 1H), 7.20 (d, 1H), 3.45 (m, 1H), 1.86 (m, 1H), 1.76 (m, 1H), 1.66 (m, 1H), 1.59 (m, 1H), 1.38 (m, 3H); LC/MS: M+H=480.14.

Chiralpak-IG (4.6×250) mm 5 um, 3% EtOH in CH₂Cl₂ (v/v), Flow Rate: 1.0 mL/min, Rt=15.65 min

### Examples 141 and 142 (E)-1⁷-(3-cyclopropyl-2-oxoimidazolidin-1-yl)-11-methyl-1²,1³-dihydro-1H-2,6-diaza-1(2,1)-benzo[d]imidazola-4(1,3)-benzenacycloundecaphane-3,5-dione

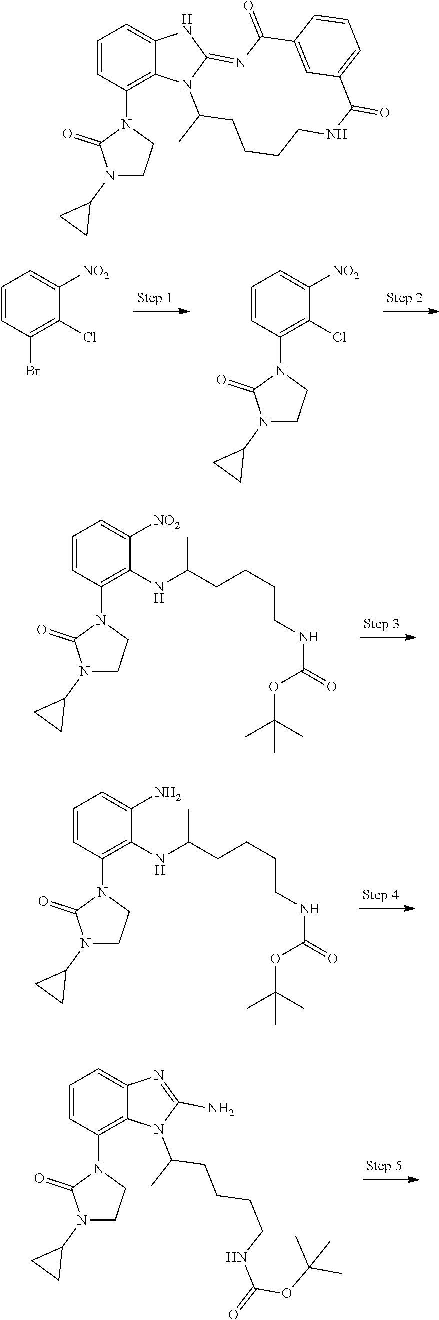

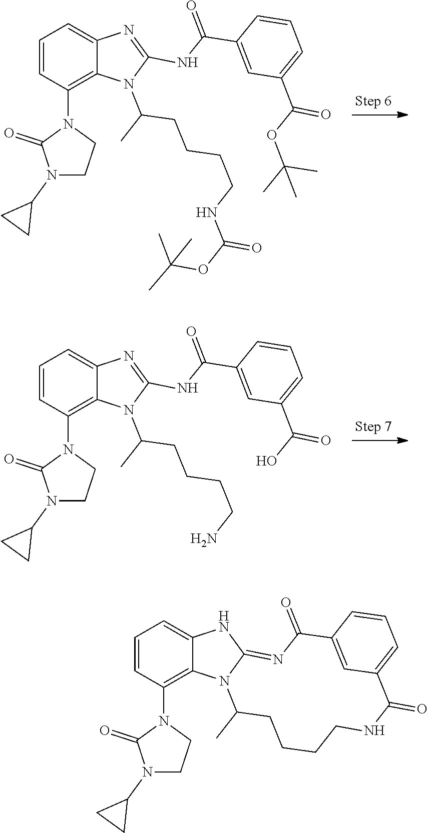 [SMILES: CC(CCCCNC(=O)OC(C)(C)C)n1c(NC(=O)c2cccc(C(=O)OC(C)(C)C)c2)nc2cccc(N3CCN(C4CC4)C3=O)c21 ; CC(CCCCN)n1c(NC(=O)c2cccc(C(=O)O)c2)nc2cccc(N3CCN(C4CC4)C3=O)c21 ; CC1CCCCNC(=O)c2cccc(c2)C(=O)/N=C2\Nc3cccc(N4CCN(C5CC5)C4=O)c3N21]

### Step 1: Preparation of 1-(2-chloro-3-nitrophenyl)-3-cyclopropylimidazolidin-2-one

A solution of 1-bromo-2-chloro-3-nitrobenzene (3 g, 12.7 mmol), 1-cyclopropylimidazolidin-2-one (4.8 g, 38.1 mmol) and Cs₂CO₃ (12.4 g, 38.1 mmol) in dioxane (8.0 ml) was degassed. Xantphos (1.47 g, 2.54 mmol) and Pd(OAc)₂ (285 mg, 1.27 mmol) were added and the resulting mixture was degassed for 10 min before heating at 100° C. for 3 h. The reaction mixture was cooled, filtered through celite with EtOAc. The filtrate was concentrated under reduced pressure, diluted with water, and extracted with EtOAc. The organic phase was dried and concentrated under reduced pressure. The crude product was purified by flash chromatography (SiO2, 80% EtOAc in heptane) to give 1-(2-chloro-3-nitrophenyl)-3-cyclopropylimidazolidin-2-one (3.0 g, 66%).

¹H NMR (DMSO-d₆) δ: 7.94 (d, 1H), 7.74 (d, 1H), 7.61 (m, 1H), 3.72 (m, 2H), 3.48 (m, 2H), 0.66 (m, 4H); LC/MS: M+H=282.00.

### Step 2: Preparation of tert-butyl (5-((2-(3-cyclopropyl-2-oxoimidazolidin-1-yl)-6-nitrophenyl)amino)hexyl)carbamate

To a stirred solution of 1-(2-chloro-3-nitrophenyl)-3-cyclopropylimidazolidin-2-one (1.5 g, 5.32 mmol) in DMF (12.6 ml) was added K₂CO₃ (2.21 g, 16 mmol) and tert-butyl (5-aminohexyl)carbamate (3.46 g, 16 mmol). The reaction mixture was heated at 100° C. for 3 days. The reaction mixture was cooled, diluted with ice cold water and extracted with EtOAc (30 mL×3). The combined organic layers was dried and concentrated under reduced pressure. The crude product was purified by flash chromatography (SiO2, 60% EtOAc in pet. ether) to give tert-butyl (5-((2-(3-cyclopropyl-2-oxoimidazolidin-1-yl)-6-nitrophenyl)amino)hexyl)carbamate (1.4 g, 56%).

¹H NMR (DMSO-d₆) δ: 7.94 (d, 1H), 7.59 (d, 1H), 6.87 (m, 2H), 6.75 (m, 1H), 3.60 (m, 1H), 3.52 (m, 1H), 3.45 (m, 2H), 2.86 (m, 2H), 1.46 (m, 1H), 1.36 (s, 9H), 1.05 (d, 3H); LC/MS: M+H=462.30.

### Step 3: Preparation of tert-butyl (5-((2-amino-6-(3-cyclopropyl-2-oxoimidazolidin-1-yl)phenyl)amino)hexyl)carbamate

To a stirred solution of tert-butyl (5-((2-(3-cyclopropyl-2-oxoimidazolidin-1-yl)-6-nitrophenyl)amino)hexyl)carbamate (1.4 g, 3.03 mmol) in MeOH (100 ml) was added Raney-Ni (1.0 g, 17 mmol). The reaction mixture was stirred at rt under a hydrogen atmosphere for 3 h. The reaction mixture was filtered through celite bed with MeOH, and the filtrate was concentrated under reduced pressure. The crude product was purified by flash chromatography (SiO2, 50% EtOAc in heptane) to give tert-butyl (5-((2-amino-6-(3-cyclopropyl-2-oxoimidazolidin-1-yl)phenyl)amino)hexyl)carbamate (1.3 g, 40%).

¹H NMR (DMSO-d₆) δ: 6.77 (m, 1H), 6.72 (m, 1H), 6.53 (d, 1H), 6.46 (d, 1H), 3.65 (m, 2H), 3.52 (m, 2H), 1.36 (s, 9H), 0.89 (d, 3H); LC/MS: M+H=432.25.

### Step 4: Preparation of tert-butyl (5-(2-amino-7-(3-cyclopropyl-2-oxoimidazolidin-1-yl)-1H-benzo[d]imidazol-1-yl)hexyl)carbamate

To a stirred solution of tert-butyl (5-((2-amino-6-(3-cyclopropyl-2-oxoimidazolidin-1-yl)phenyl)amino)hexyl)carbamate (1.0 g, 1.16 mmol) in MeOH (5 ml) and water (5 ml) was added cyanogen bromide (184 mg, 1.74 mmol). The reaction mixture was stirred at 50° C. for 2 h. The reaction mixture was concentrated and neutralized with NaHCO₃ (sat.) and extracted with 10% MeOH/CH₂Cl₂ (15 ml×3). The combined organic layer was concentrated under reduced pressure. The crude product was purified by flash chromatography (SiO2, 5% MeOH/CH₂Cl₂) to give tert-butyl (5-(2-amino-7-(3-cyclopropyl-2-oxoimidazolidin-1-yl)-1H-benzo[d]imidazol-1-yl)hexyl)carbamate (0.3 g, 49%).

¹H NMR (DMSO-d₆) δ: 7.04 (m, 1H), 6.92 (m, 1H), 6.72 (m, 2H), 6.19 (m, 1H), 3.58 (m, 1H), 3.46 (m, 2H), 1.81 (m, 2H), 1.39 (d, 3H), 0.64 (m, 4H); LC/MS: M+H=457.55.

### Step 5: Preparation of tert-butyl 3-((1-(6-((tert-butoxycarbonyl)amino)hexan-2-yl)-7-(3-cyclopropyl-2-oxoimidazolidin-1-yl)-1H-benzo[d]imidazol-2-yl)carbamoyl)benzoate

To a stirred solution of 3-((tert-butoxy)carbonyl)benzoic acid (292 mg, 1.31 mmol), TCFH (460 mg, 1.64 mmol) and 1-methyl-1H-imidazole (262 μL, 3.29 mmol) in MeCN (4 ml) was added tert-butyl (5-(2-amino-7-(3-cyclopropyl-2-oxoimidazolidin-1-yl)-1H-benzo[d]imidazol-1-yl)hexyl)carbamate (0.3 g, 657 μmol). The reaction mixture was stirred at rt for 8 h. The reaction mixture was concentrated under reduced pressure, and ice cold water was added. The formed solid was collected by filtration. The crude product was purified by flash chromatography (SiO2, 80% EtOAc in heptane) to give tert-butyl 3-((1-(6-((tert-butoxycarbonyl)amino)hexan-2-yl)-7-(3-cyclopropyl-2-oxoimidazolidin-1-yl)-1H-benzo[d]imidazol-2-yl)carbamoyl)benzoate (0.3 g, 40%).

¹H NMR (DMSO-d₆) δ: 8.81 (s, 1H), 8.36 (d, 1H), 8.04 (d, 1H), 7.64 (m, 1H), 7.52 (m, 1H), 7.21 (m, 1H), 3.63 (m, 1H), 3.51 (m, 2H), 2.83 (m, 2H), 1.72 (m, 2H), 1.58 (s, 9H), 1.32 (m, 3H), 0.66 (m, 4H); LC/MS: M+H=661.45.

### Step 6: Preparation of 3-((1-(6-aminohexan-2-yl)-7-(3-cyclopropyl-2-oxoimidazolidin-1-yl)-1H-benzo[d]imidazol-2-yl)carbamoyl)benzoic acid

To a solution of tert-butyl 3-((1-(6-((tert-butoxycarbonyl)amino)hexan-2-yl)-7-(3-cyclopropyl-2-oxoimidazolidin-1-yl)-1H-benzo[d]imidazol-2-yl)carbamoyl)benzoate (300 mg, 454 μmol) in CH₂Cl₂ was added TFA (1.0 ml). The reaction mixture was stirred at rt for 16 h. The reaction mixture was concentrated under reduced pressure and the residue was washed with diethyl ether. The product (200 mg) was used without further purification.

¹H NMR (DMSO-d₆) δ: 8.78 (s, 1H), 8.40 (d, 1H), 8.10 (d, 1H), 7.63 (m, 2H), 7.29 (m, 1H), 3.91 (m, 1H), 3.64 (m, 2H), 3.53 (m, 2H), 1.71 (m, 3H), 0.67 (m, 4H);

LC/MS: M+H=505.30.

### Step 7: Preparation of (E)-1⁷-(3-cyclopropyl-2-oxoimidazolidin-1-yl)-11-methyl-1²,1³-dihydro-1¹H-2,6-diaza-1(2,1)-benzo[d]imidazola-4(1,3)-benzenacycloundecaphane-3,5-dione

To a solution of 3-((1-(6-aminohexan-2-yl)-7-(3-cyclopropyl-2-oxoimidazolidin-1-yl)-1H-benzo[d]imidazol-2-yl)carbamoyl)benzoic acid (300 mg, 595 μmol) in MeCN (700 ml) and DMF (3 ml) was added TCFH (417 mg, 1.49 mmol) at 0° C. The reaction mixture was stirred at 0° C. for 30 minutes and then 1-methyl-1H-imidazole (237 μl, 2.97 mmol) was added. The resulting solution was stirred at 0° for 3 h. The reaction mixture was evaporated under reduced pressure and to the residue was added ice cold water. The formed precipitate was collected by filtration. The crude product was purified by flash chromatography (SiO2, 10% 1:1 EtOAC/MeOH in CH₂Cl₂) to give (E)-1⁷-(3-cyclopropyl-2-oxoimidazolidin-1-yl)-11-methyl-1²,1³-dihydro-1¹H-2,6-diaza-1(2,1)-benzo[d]imidazola-4(1,3)-benzenacycloundecaphane-3,5-dione. The enantiomers were separated by chiral chromatography (Chiralpak IG (250*4.6) mm, 5 um, isopropyl alcohol/EtOAc 50:50, Flow Rate: 1.0 mL/min) to afford enantiomer 1 (17 mg) and enantiomer 2 (16 mg).

### Example 141, Enantiomer 1

¹H NMR (DMSO-d₆) @ t=352.2 K δ: 8.86 (s, 1H), 8.10 (d, 1H), 7.74 (d, 1H), 7.57 (m, 1H), 7.48 (m, 2H), 7.20 (m, 1H), 7.09 (d, 1H), 5.10 (bs, 1H), 3.72 (bs, 2H), 3.52 (m, 3H), 1.67 (d, 3H); LC/MS: M+H=487.30.

Chiralpak IG (250*4.6) mm, 5 um, isopropyl alcohol/EtOAc 50:50, Flow Rate: 1.0 mL/min, Rt=6.74 min

### Example 142, Enantiomer 2

¹H NMR (DMSO-d₆) @ t=353.2 K δ: 8.86 (s, 1H), 8.10 (d, 1H), 7.74 (d, 1H), 7.57 (m, 1H), 7.48 (m, 2H), 7.20 (m, 1H), 7.09 (d, 1H), 5.10 (bs, 1H), 3.72 (bs, 2H), 3.52 (m, 3H), 1.67 (d, 3H); LC/MS: M+H=487.25.

Chiralpak IG (250*4.6) mm, 5 um, isopropyl alcohol/EtOAc 50:50, Flow Rate: 1.0 mL/min, Rt=8.60 min

### Examples 143 and 144 (E)-11-methyl-1⁷-(3-(oxetan-3-yl)-2-oxoimidazolidin-1-yl)-1²,1³-dihydro-1¹H-2,6-diaza-1(2,1)-benzo[d]imidazola-4(1,3)-benzenacycloundecaphane-3,5-dione

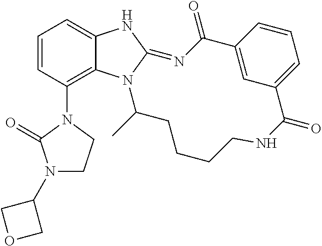 [SMILES: CC1CCCCNC(=O)c2cccc(c2)C(=O)/N=C2\Nc3cccc(N4CCN(C5COC5)C4=O)c3N21]

Examples 143 and 144 were prepared from 1-bromo-2-chloro-3-nitrobenzene and 1-(oxetan-3-yl)imidazolidin-2-one analogous to the preparation of Examples 141 and 142.

The enantiomers of (E)-11-methyl-1⁷-(3-(oxetan-3-yl)-2-oxoimidazolidin-1-yl)-1²,1³-dihydro-1¹H-2,6-diaza-1(2,1)-benzo[d]imidazola-4(1,3)-benzenacycloundecaphane-3,5-dione were separated using chiral chromatography.

### Example 143, Enantiomer 1

¹H NMR (DMSO-d₆) δ: 8.86 (s, 1H), 8.10 (d, 1H), 7.75 (d, 1H), 7.57 (m, 1H), 7.49 (m, 1H), 7.21 (m, 2H), 7.124 (m, 1H), 4.95 (m, 1H), 4.83 (m, 2H), 4.74 (m, 2H), 1.65 (d, 3H); LC/MS: M+H=503.35.

Chiralpak-IG (4.6×250 mm) 5μ, 0.1% AH in IPA/EtOAc=(30:70) (v/v), flow rate: 1 ml/min, Rt=8.95 min

### Example 144, Enantiomer 2

¹H NMR (DMSO-d₆) δ: 8.86 (s, 1H), 8.10 (d, 1H), 7.75 (d, 1H), 7.57 (m, 1H), 7.49 (m, 1H), 7.21 (m, 2H), 7.124 (m, 1H), 4.95 (m, 1H), 4.83 (m, 2H), 4.74 (m, 2H), 1.66 (d, 3H); LC/MS: M+H=503.30.

Chiralpak-IG (4.6×250 mm) 5μ, 0.1% AH in IPA/EtOAc=(30:70) (v/v), flow rate: 1 ml/min, Rt=11.21 min

### Example 145: (S,E)-N,N,11-trimethyl-3,5-dioxo-1²,1³-dihydro-1¹H-2,6-diaza-1(2,1)-imidazo[4,5-c]pyridina-4(1,3)-benzenacycloundecaphane-1⁷-carboxamide

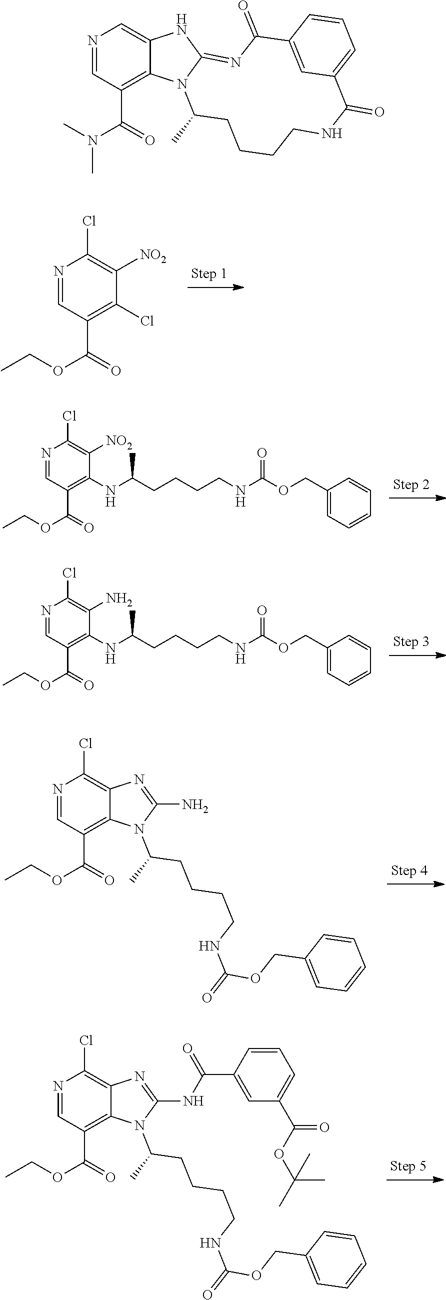 [SMILES: C[C@H]1CCCCNC(=O)c2cccc(c2)C(=O)/N=C2\Nc3cncc(C(=O)N(C)C)c3N21 ; CCOC(=O)c1cnc(Cl)c([N+](=O)[O-])c1Cl ; CCOC(=O)c1cnc(Cl)c([N+](=O)[O-])c1N[C@@H](C)CCCCNC(=O)OCc1ccccc1 ; CCOC(=O)c1cnc(Cl)c(N)c1N[C@@H](C)CCCCNC(=O)OCc1ccccc1 ; CCOC(=O)c1cnc(Cl)c2nc(N)n([C@@H](C)CCCCNC(=O)OCc3ccccc3)c12 ; CCOC(=O)c1cnc(Cl)c2nc(NC(=O)c3cccc(C(=O)OC(C)(C)C)c3)n([C@@H](C)CCCCNC(=O)OCc3ccccc3)c12]

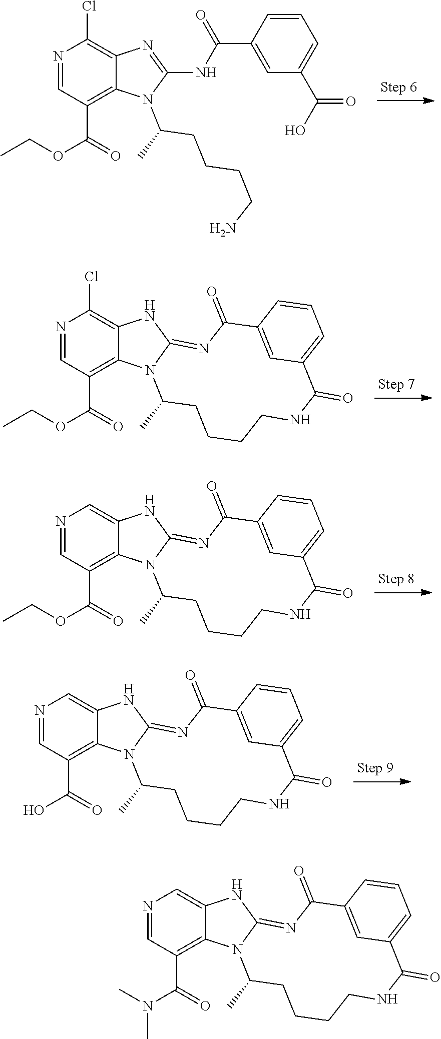 [SMILES: CCOC(=O)c1cnc(Cl)c2nc(NC(=O)c3cccc(C(=O)O)c3)n([C@@H](C)CCCCN)c12 ; CCOC(=O)c1cnc(Cl)c2c1N1/C(=N/C(=O)c3cccc(c3)C(=O)NCCCC[C@@H]1C)N2 ; CCOC(=O)c1cncc2c1N1/C(=N/C(=O)c3cccc(c3)C(=O)NCCCC[C@@H]1C)N2 ; C[C@H]1CCCCNC(=O)c2cccc(c2)C(=O)/N=C2\Nc3cncc(C(=O)O)c3N21 ; C[C@H]1CCCCNC(=O)c2cccc(c2)C(=O)/N=C2\Nc3cncc(C(=O)N(C)C)c3N21]

### Step 1: Preparation of ethyl (S)-4-((6-(((benzyloxy)carbonyl)amino)hexan-2-yl)amino)-6-chloro-5-nitronicotinate

To a solution of ethyl 4,6-dichloro-5-nitropyridine-3-carboxylate (5 g, 18.9 mmol) and benzyl N-(5S)-5-aminohexyl]carbamate (5.67 g, 21.5 mmol) in DMF (50 ml) was added N,N-diisopropylethylamine 9.86 ml, 56.6 mmol). The reaction mixture was stirred at 80° C. for 4 h. The reaction mixture was quenched with ice-cold water and extracted with EtOAc. The organic layer was washed with brine solution, dried over Na₂SO₄ and concentrated under reduced pressure. The product was used without further purification.

¹H NMR (DMSO-d₆) δ: 8.66 (s, 1H), 7.95 (s, 1H), 7.33 (m, 5H), 4.99 (s, 2H), 4.37 (m, 2H), 3.22 (m, 1H), 2.97 (m, 2H), 1.34 (m, 3H), 1.13 (d, 3H); LC/MS: M+H=479.15.

### Step 2: Preparation of ethyl (S)-5-amino-4-((6-(((benzyloxy)carbonyl)amino)hexan-2-yl)amino)-6-chloronicotinate

A solution of ethyl (S)-4-((6-(((benzyloxy)carbonyl)amino)hexan-2-yl)amino)-6-chloro-5-nitronicotinate (11 g, 23 mmol) and Raney-nickel (1.35 g, 23 mmol) in ethanol (100 ml) was stirred under an atmosphere of hydrogen at rt for 4 h. The reaction mixture was filtered, washed with EtOH and concentrated in vacuum. The crude residue was purified by flash chromatography (SiO₂, 0 to 80% EtOAc in heptane) to afford of ethyl (S)-5-amino-4-((6-(((benzyloxy)carbonyl)amino)hexan-2-yl)amino)-6-chloronicotinate (4.3 g, 30%).

LC/MS: M+H=449.30; Rt=2.25 min.

### Step 3: Preparation of ethyl (S)-2-amino-1-(6-(((benzyloxy)carbonyl)amino)hexan-2-yl)-4-chloro-1H-imidazo[4,5-c]pyridine-7-carboxylate

To a stirred solution of ethyl (S)-5-amino-4-((6-(((benzyloxy)carbonyl)amino)hexan-2-yl)amino)-6-chloronicotinate (2.0 g, 4.45 mmol) in MeCN (5 ml), tert-butanol (10 ml) and water (5 ml) was added cyanogen bromide (1.42 g, 13.4 mmol). The reaction mixture was stirred at 60° C. for 32 h. The reaction mixture was quenched with NaHCO_{3(aq)} and extracted with CH₂Cl₂. The organic layer was washed with brine solution, dried with Na₂SO₄ and concentrated under reduced pressure. The crude was purified by flash chromatography (SiO₂, 5% MeOH in CH₂Cl₂) to afford ethyl (S)-2-amino-1-(6-(((benzyloxy)carbonyl)amino)hexan-2-yl)-4-chloro-1H-imidazo[4,5-c]pyridine-7-carboxylate (550 mg, 16%).

LC/MS: M+H=474.10; Rt=1.94 min.

### Step 4: Preparation ethyl (S)-1-(6-(((benzyloxy)carbonyl)amino)hexan-2-yl)-2-(3-(tert-butoxycarbonyl)benzamido)-4-chloro-1H-imidazo[4,5-c]pyridine-7-carboxylate

A solution of 3-(tert-butoxycarbonyl)benzoic acid (317 mg, 1.42 mmol), TCFH (533 mg, 1.9 mmol) and 1-methyl-1H-imidazole (378 μl, 4.75 mmol) in MeCN (22.5 ml) was stirred for 5 min before a solution of ethyl (S)-2-amino-1-(6-(((benzyloxy)carbonyl)amino)hexan-2-yl)-4-chloro-1H-imidazo[4,5-c]pyridine-7-carboxylate (450 mg, 949 μmol) in MeCN (22.5 ml) was added. The reaction mixture was stirred at rt for 16 h. The reaction mixture was quenched with water. The aqueous layer was extracted with EtOAc, dried with Na₂SO) and concentrated under reduced pressure. The crude residue was purified by flash chromatography (SiO₂, 0 to 100% EtOAc in heptane) to afford ethyl (S)-1-(6-(((benzyloxy)carbonyl)amino)hexan-2-yl)-2-(3-(tert-butoxycarbonyl)benzamido)-4-chloro-1H-imidazo[4,5-c]pyridine-7-carboxylate (580 mg, 63%).

¹H NMR (DMSO-d₆) δ: 8.53 (d, 1H), 8.23 (m, 1H), 7.75 (m, 1H), 7.32 (m, 5H), 7.13 (m, 1H), 5.03 (m, 3H), 4.41 (m, 2H), 2.08 (m, 2H), 1.95 (m, 1H), 1.58 (s, 9H), 1.36 (m, 3H); LC/MS: M+H=678.90.

### Step 5: Preparation of (S)-3-((1-(6-aminohexan-2-yl)-4-chloro-7-(ethoxycarbonyl)-1H-imidazo[4,5-c]pyridin-2-yl)carbamoyl)benzoic acid

A solution of ethyl (S)-1-(6-(((benzyloxy)carbonyl)amino)hexan-2-yl)-2-(3-(tert-butoxycarbonyl)benzamido)-4-chloro-1H-imidazo[4,5-c]pyridine-7-carboxylate (580 mg, 811 μmol) and TFA (621 μl, 8.11 mmol) in CH₂Cl₂ (10 ml) was stirred at rt for 16 h. The reaction mixture was concentrated under reduced pressure. The residue was evaporated to dryness from diethyl ether. The product was used without further purification.

LC/MS: M+H=487.95; Rt=1.31 min.

### Step 6: Preparation of ethyl (S,E)-1⁴-chloro-11-methyl-3,5-dioxo-1²,1³-dihydro-1¹H-2,6-diaza-1(2,1)-imidazo[4,5-c]pyridina-4(1,3)-benzenacycloundecaphane-1⁷-carboxylate

A solution of TCFH (373 mg, 1.33 mmol) and 1-methyl-1H-imidazole (318 μl, 3.98 mmol) in MeCN (200 ml) was cooled to 0° C. and stirred for 15 min. To this solution was added a solution of (S)-3-((1-(6-aminohexan-2-yl)-4-chloro-7-(ethoxycarbonyl)-1H-imidazo[4,5-c]pyridin-2-yl)carbamoyl)benzoic acid (450 mg, 664 μmol) in MeCN (300 ml) and DMF (15 ml). The reaction mixture was stirred at 0° C. for 4 h. Reaction mixture was quenched with water (10 ml) and concentrated under reduced pressure to afford as brown sticky liquid crude. This crude mixture was diluted with water and the formed precipitate was collected by filtration, washed with cold water and dried under reduced pressure. The crude product was triturated with CH₂Cl₂ to afford ethyl (SE)-1⁴-chloro-11-methyl-3,5-dioxo-1²,1³-dihydro-1¹H-2,6-diaza-1(2,1)-imidazo[4,5-c]pyridina-4(1,3)-benzenacycloundecaphane-1⁷-carboxylate (210 mg, 49%).

LC/MS: M+H=470.20; Rt=2.01 min,

### Step 7: Preparation of ethyl (S,E)-11-methyl-3,5-dioxo-1²,1³-dihydro-1¹H-2,6-diaza-1(2,1)-imidazo[4,5-c]pyridina-4(1,3)-benzenacycloundecaphane-1⁷-carboxylate

To a solution of ethyl (S,E)-1⁴-chloro-11-methyl-3,5-dioxo-1²,1³-dihydro-1¹H-2,6-diaza-1(2,1)-imidazo[4,5-c]pyridina-4(1,3)-benzenacycloundecaphane-1⁷-carboxylate (190 mg, 295 μmol) in dioxane (2.08 ml), THF (1.04 ml) and water (693 μl) was added ammonium chloride (237 mg, 4.43 mmol) followed by portion wise addition of zinc (232 mg, 3.54 mmol). The reaction mixture was stirred at RT for 16 h. Reaction mixture was filtered through celite, washed with 10% MeOH in CH₂Cl₂. The filtrate was washed with water (10 ml) and then brine (10 ml), dried (Na₂SO₄) and concentrated under reduced pressure. The crude was purified by using flash chromatography (SiO₂, 0-10% MeOH in CH₂Cl₂) to afford ethyl (S,E)-11-methyl-3,5-dioxo-1²,1³-dihydro-1¹H-2,6-diaza-1(2,1)-imidazo[4,5-c]pyridina-4(1,3)-benzenacycloundecaphane-1⁷-carboxylate (130 mg, 73%).

¹H NMR (DMSO-d₆) δ: 9.28 (s, 1H), 8.80 (m, 1H), 8.57 (s, 1H), 8.09 (m, 1H), 7.75 (m, 1H), 7.57 (m, 1H), 1.63 (m, 3H); LC/MS: M+H=436.20.

### Step 8: Preparation of (S,E)-11-methyl-3,5-dioxo-1²,1³-dihydro-1¹H-2,6-diaza-1(2,1)-imidazo[4,5-c]pyridina-4(1,3)-benzenacycloundecaphane-1⁷-carboxylic acid

A solution of ethyl (S,E)-11-methyl-3,5-dioxo-1²,1³-dihydro-1¹H-2,6-diaza-1(2,1)-imidazo[4,5-c]pyridina-4(1,3)-benzenacycloundecaphane-1⁷-carboxylate (110 mg, 184 μmol) and LiOH (22 mg, 922 μmol) in dioxane (3 ml) and water (1 ml) was stirred at rt for 16 h. The reaction mixture was concentrated under reduced pressure. The crude residue was acidified by 1M HCl. The precipitate was collected by filtration, washed with water, and dried to afford (S,E)-11-methyl-3,5-dioxo-1²,1³-dihydro-1¹H-2,6-diaza-1(2,1)-imidazo[4,5-c]pyridina-4(1,3)-benzenacycloundecaphane-1⁷-carboxylic acid (80 mg, 45%).

LC/MS: M+H=407.95; Rt=0.96 min.

### Step 9: Preparation of (S,E)-N,N,11-trimethyl-3,5-dioxo-1²,1³-dihydro-1¹H-2,6-diaza-1(2,1)-imidazo[4,5-c]pyridina-4(1,3)-benzenacycloundecaphane-1⁷-carboxamide

To a stirred solution of (S,E)-11-methyl-3,5-dioxo-1²,1³-dihydro-1¹H-2,6-diaza-1(2,1)-imidazo[4,5-c]pyridina-4(1,3)-benzenacycloundecaphane-1⁷-carboxylic acid (80 mg, 82 μmol) in MeCN (3 ml) and DMF (0.5 ml) was added TCFH (46 mg, 165 μmol) and 1-methyl-1H-imidazole (33 μl, 412 μmol). The reaction mixture was stirred at 0° C. for 15 min before 2M dimethylamine in THF (554 μl, 412 μmo) was added dropwise. The reaction mixture was stirred at rt for 16 h. The reaction mixture was concentrated under reduced pressure and the crude was triturated with water. The isolated crude product was purified by preparative HPLC to afford (S,E)-N,N,11-trimethyl-3,5-dioxo-1²,1³-dihydro-1¹H-2,6-diaza-1(2,1)-imidazo[4,5-c]pyridina-4(1,3)-benzenacycloundecaphane-1⁷-carboxamide (4 mg, 10%).

¹H NMR (DMSO-d₆) @ 353.7 K δ: 8.88 (m, 1H), 8.70 (s, 1H), 8.20 (s, 1H), 8.11 (d, 1H), 7.77 (d, 1H), 7.59 (m, 1H), 7.54 (m, 1H), 3.11 (s, 3H), 2.92 (s, 3H), 1.90 (m, 3H), 1.59 (d, 3H); LC/MS: M+H=435.25.

### Example 146: (11S,E)-1⁷-(3-fluoro-2-oxopyrrolidin-1-yl)-11-methyl-1²,1³-dihydro-1¹H-2,6-diaza-1(2,1)-benzo[d]imidazola-4(1,3)-benzenacycloundecaphane-3,5-dione

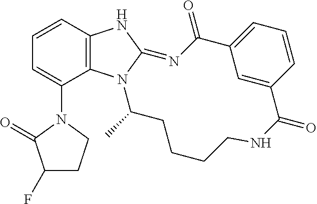 [SMILES: C[C@H]1CCCCNC(=O)c2cccc(c2)C(=O)/N=C2\Nc3cccc(N4CCC(F)C4=O)c3N21]

Example 146 was prepared from (S,E)-1⁷-bromo-11-methyl-1²,1³-dihydro-1¹H-2,6-diaza-1(2,1)-benzo[d]imidazola-4(1,3)-benzenacycloundecaphane-3,5-dione and 3-fluoropyrrolidin-2-one analogous to the preparation of Example 126 and 127.

¹H NMR (DMSO-d₆) @ 353.2 K δ: 8.83 (bs, 1H), 8.10 (d, 1H), 7.75 (d, 1H), 7.57 (m, 2H), 7.49 (bs, 1H), 7.26 (m, 1H), 7.14 (m, 1H), 5.37 (m, 1H), 3.80 (m, 2H), 3.56 (m, 1H), 1.95 (m, 1H); LC/MS: M+H=464.25.

### Example 147: (S,E)-11-methyl-1⁷-(3-methyl-1,2,4-oxadiazol-5-yl)-1²,1³-dihydro-1¹H-2,6-diaza-1(2,1)-benzo[d]imidazola-4(1,3)-benzenacycloundecaphane-3,5-dione

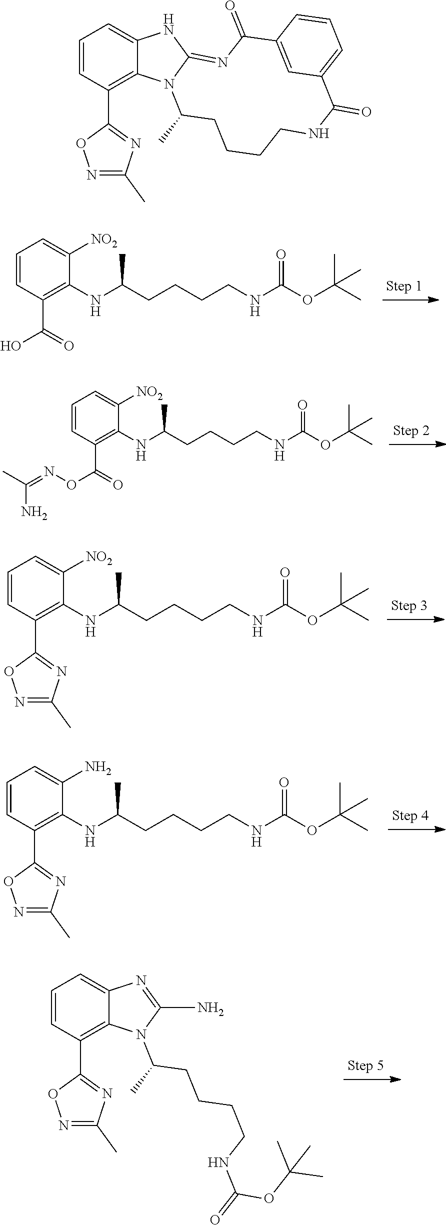

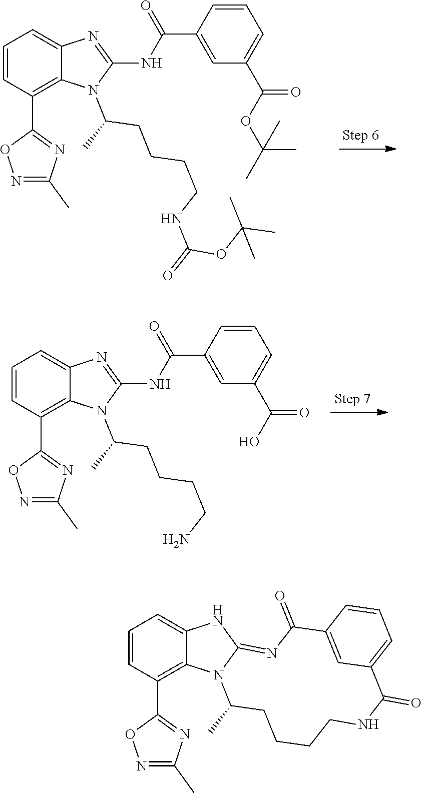 [SMILES: Cc1noc(-c2cccc3nc(NC(=O)c4cccc(C(=O)OC(C)(C)C)c4)n([C@@H](C)CCCCNC(=O)OC(C)(C)C)c23)n1 ; Cc1noc(-c2cccc3nc(NC(=O)c4cccc(C(=O)O)c4)n([C@@H](C)CCCCN)c23)n1 ; Cc1noc(-c2cccc3c2N2/C(=N/C(=O)c4cccc(c4)C(=O)NCCCC[C@@H]2C)N3)n1]

### Step 1: Preparation of tert-butyl (S)-(5-((2-((((1-aminoethylidene)amino)oxy)carbonyl)-6-nitrophenyl)amino)hexyl)carbamate

To a stirred solution of (S)-2-((6-((tert-butoxycarbonyl)amino)hexan-2-yl)amino)-3-nitrobenzoic acid (690 mg, 1.74 mmol), N′-hydroxyacetimidamide (259 mg, 3.49 mmol) and N,N-diisopropylethylamine (0.97 ml, 5.23 mmol) in DMF (8 ml) was added TCFH (1.33 g, 3.49 mmol) at rt. The reaction mixture was stirred at rt for 16 h and the quenched by the addition of water. The aqueous layer was extracted with EtOAc, dried (Na₂SO₄) and concentrated under reduced pressure. The crude product was used without further purification.

¹H NMR (DMSO-d₆) δ: 8.20 (d, 1H), 8.06 (d, 1H), 8.00 (m, 1H), 6.80 (m, 1H), 6.73 (m, 1H), 6.53 (bs, 1H), 3.23 (m, 1H), 2.85 (m, 2H), 2.69 (s, 3H), 1.44 (m, 1H), 1.36 (s, 9H), 1.05 (d, 3H); LC/MS: M+H=438.20.

### Step 2: Preparation of tert-butyl (S)-(5-((2-(3-methyl-1,2,4-oxadiazol-5-yl)-6-nitrophenyl)amino)hexyl)carbamate

A solution of tert-butyl (S)-(5-((2-((((1-aminoethylidene)amino)oxy)carbonyl)-6-nitrophenyl)amino)hexyl)carbamate (550 mg, 1.26 mmol) and tetrabutylammonium fluoride (329 mg, 1.26 mmol) in THF (10 ml) was stirred at rt for 16 h. The reaction mixture was concentrated under reduced pressure and the crude residue was purified by flash chromatography (SiO₂, 0-80% EtOAc in heptane) to afford tert-butyl (S)-(5-((2-(3-methyl-1,2,4-oxadiazol-5-yl)-6-nitrophenyl)amino)hexyl)carbamate (480 mg, 90%).

¹H NMR (DMSO-d₆) δ: 8.19 (d, 1H), 8.07 (d, 1H), 8.03 (m, 1H), 6.97 (m, 1H), 3.12 (m, 1H), 2.85 (m, 2H), 2.48 (s, 3H), 1.45 (m, 2H), 1.35 (s, 9H), 1.24 (m, 2H), 1.06 (d, 3H); LC/MS: M+H=420.28; Rt=2.37 min.

### Step 3: Preparation of tert-butyl (S)-(5-((2-amino-6-(3-methyl-1,2,4-oxadiazol-5-yl)phenyl)amino)hexyl)carbamate

A solution of tert-butyl (S)-(5-((2-(3-methyl-1,2,4-oxadiazol-5-yl)-6-nitrophenyl)amino)hexyl)carbamate (420 mg, 1.00 mmol), zinc (655 mg, 10 mmol) and ammonium chloride (536 mg, 10 mmol) in EtOH (8 ml) and water (2 ml) was stirred at rt for 2 h. The reaction mixture was concentrated under reduced pressure and the crude residue was quenched by the addition of water. The aqueous layer was extracted with EtOAc, dried (Na₂SO₄) and concentrated under reduced pressure. The crude residue was purified by flash chromatography (SiO₂, 0-80% EtOAc in heptane) to afford tert-butyl (S)-(5-((2-amino-6-(3-methyl-1,2,4-oxadiazol-5-yl)phenyl)amino)hexyl)carbamate (370 mg, 72%).

¹H NMR (DMSO-d₆) δ: 7.19 (m, 1H), 6.88 (m, 2H), 6.73 (m, 1H), 5.96 (d, 1H), 4.81 (s, 2H), 3.48 (m, 1H), 2.86 (m, 2H), 2.43 (s, 3H), 1.36 (s, 9H), 0.88 (d, 3H); LC/MS: M+H=390.45.

### Step 4: Preparation tert-butyl (S)-(5-(2-amino-7-(3-methyl-1,2,4-oxadiazol-5-yl)-1H-benzo[d]imidazol-1-yl)hexyl)carbamate

A solution of tert-butyl (S)-(5-((2-amino-6-(3-methyl-1,2,4-oxadiazol-5-yl)phenyl)amino)hexyl)carbamate (320 mg, 822 μmol) cyanogen bromide (104 mg, 986 μmol) in MeOH (19.2 ml), MeCN (9.6 ml) and water (9.6 ml) was heated at 55° C. for 2 h. The reaction mixture was concentrated under reduced pressure and the crude residue was diluted with water (50 ml). The aqueous layer was washed with Et₂O (2×100 ml). The aqueous layer was basified with excess NaHCO₃ and then extracted with 5% MeOH in CH₂Cl₂. The organic phase was dried (Na₂SO₄) and concentrated under reduced pressure. The crude product was used without further purification.

¹H NMR (DMSO-d₆) δ: 7.58 (d, 1H), 7.21 (d, 1H), 7.10 (m, 1H), 6.45 (bs, 2H), 4.29 (m, 1H), 2.75 (m, 2H), 2.47 (s, 3H), 1.42 (d, 3H), 1.33 (s, 9H); LC/MS: M+H=415.25.

### Step 5: Preparation tert-butyl (S)-3-((1-(6-((tert-butoxycarbonyl)amino)hexan-2-yl)-7-(3-methyl-1,2,4-oxadiazol-5-yl)-1H-benzo[d]imidazol-2-yl)carbamoyl)benzoate

To a stirred solution of 3-(tert-butoxycarbonyl)benzoic acid (241 mg, 1.09 mmol) and 1-methyl-1H-imidazole (288 μl, 3.62 mmol) in MeCN (30 ml) was added portionwise TCFH (406 mg, 1.45 mmol). The resulting solution was stirred for 10 min at rt before a solution of tert-butyl (S)-(5-(2-amino-7-(3-methyl-1,2,4-oxadiazol-5-yl)-1H-benzo[d]imidazol-1-yl)hexyl)carbamate (300 mg, 724 μmol) in MeCN (30 ml) was added. The reaction mixture was stirred at rt for 16 h. The reaction mixture was concentrated under reduced pressure and the crude residue was diluted with cold water. The precipitate formed was filtered, washed with cold water and dried in vacuum. This crude residue was purified by flash chromatography (SiO₂, 0-80% EtOAc in heptane) to afford tert-butyl (S)-3-((1-(6-((tert-butoxycarbonyl)amino)hexan-2-yl)-7-(3-methyl-1,2,4-oxadiazol-5-yl)-1H-benzo[d]imidazol-2-yl)carbamoyl)benzoate (340 mg, 66%).

¹H NMR (DMSO-d₆) δ: 8.82 (s, 1H), 8.37 (d, 1H), 8.06 (d, 1H), 7.86 (d, 1H), 7.61 (m, 2H), 7.40 (m, 1H), 6.63 (m, 1H), 4.32 (bs, 1H), 2.74 (m, 2H), 1.69 (m, 2H), 1.58 (s, 9H), 1.36 (s, 9H), 1.22 (m, 2H); LC/MS: M+H=619.50.

### Step 6: Preparation of (S)-3-((1-(6-aminohexan-2-yl)-7-(3-methyl-1,2,4-oxadiazol-5-yl)-1H-benzo[d]imidazol-2-yl)carbamoyl)benzoic acid

To a stirred solution of tert-butyl (S)-3-((1-(6-((tert-butoxycarbonyl)amino)hexan-2-yl)-7-(3-methyl-1,2,4-oxadiazol-5-yl)-1H-benzo[d]imidazol-2-yl)carbamoyl)benzoate (300 mg, 485 μmol) CH2Cl2 (20 ml) was added TFA (276 mg, 2.42 mmol) at 0° C. The reaction mixture was stirred at rt for 4 h. The reaction mixture was concentrated under reduced pressure and the residue was triturated with diethylether to give (S)-3-((1-(6-aminohexan-2-yl)-7-(3-methyl-1,2,4-oxadiazol-5-yl)-1H-benzo[d]imidazol-2-yl)carbamoyl)benzoic acid 210 mg, 92%).

¹H NMR (DMSO-d₆) δ: 8.80 (s, 1H), 8.40 (d, 1H), 8.10 (d, 1H), 7.88 (d, 1H), 7.62 (m, 2H), 7.40 (m, 1H), 4.32 (bs, 1H), 2.65 (m, 2H), 1.95 (m, 1H), 1.70 (d, 3H), 1.17 (m, 1H), 0.96 (m, 1H); LC/MS: M+H=463.25.

### Step 7: Preparation of (S,E)-11-methyl-1⁷-(3-methyl-1,2,4-oxadiazol-5-yl)-1²,1³-dihydro-1¹H-2,6-diaza-1(2,1)-benzo[d]imidazola-4(1,3)-benzenacycloundecaphane-3,5-dione

To a stirred solution of 1-methyl-1H-imidazole (86 μl, 1.08 mmol) and TCFH (121 mg, 432 μmol) in MeCN (702 ml) was added dropwise a solution of (S)-3-((1-(6-aminohexan-2-yl)-7-(3-methyl-1,2,4-oxadiazol-5-yl)-1H-benzo[d]imidazol-2-yl)carbamoyl)benzoic acid (100 mg, 216 μmol) in MeCN (250 ml) at 0° C. The reaction mixture was stirred at rt for 16 h. The reaction mixture was quenched by the addition of water and concentrated under reduced pressure. The residue was diluted with water and the formed precipitate was collected by filtration. The crude product was purified by preparative HPLC to afford (S,E)-11-methyl-1⁷-(3-methyl-1,2,4-oxadiazol-5-yl)-1²,1³-dihydro-1¹H-2,6-diaza-1(2,1)-benzo[d]imidazola-4(1,3)-benzenacycloundecaphane-3,5-dione (89 mg, 92%).

¹H NMR (DMSO-d₆) @ 353.1 K δ: 8.88 (bs, 1H), 8.11 (d, 1H), 7.81 (d, 1H), 7.77 (d, 1H), 7.58 (m, 2H), 7.51 (m, 1H), 7.38 (m, 1H), 3.50 (s, 3H), 1.95 (m, 2H), 1.84 (m, 1H), 1.55 (m, 1H), 1.49 (d, 3H); LC/MS: M+H=445.30.

### Physical-Chemical Example 1—Solubility Assay

The aqueous solubility of selected example compounds was determined as follows; 6 μL of DMSO stock solution of the selected example was added to 594 μL phosphate buffer (100 mM, pH 7.4). The resulting mixture was equilibrated over night (20-24 h, Alligator Shaker, room temperature). The mixture was then filtered (Millex LH 0.45 μm) and the concentration/solubility was determined by LC-UV quantification. The solubility results are shown in Table 1 below.

TABLE 1
Solubility of selected example compounds in phosphate buffer

| Example | Solubility (μM) |
|---|---|
| takinib | 0.9 |
| 1 | 1.2 |
| 2 | 0.5 |
| 3 | 31.5 |
| 4 | 0.2 |
| 5 | 15.2 |
| 6 | 2.5 |
| 7 | 0.9 |
| 8 | 92.7 |
| 9 | 30.9 |
| 10 | 28.0 |
| 11 | 4.8 |
| 12 | 31.1 |
| 13 | 0.2 |
| 14 | 0.3 |
| 15 | 90.1 |
| 16 | 78.8 |
| 17 | 93.6 |
| 18 | 95.0 |
| 19 | 8.5 |
| 20 | 35.0 |
| 21 | 85.1 |
| 22 | 88.5 |
| 23 | 83.3 |
| 24 | 26.2 |
| 25 | 82.0 |
| 26 | 78.7 |
| 27 | 61.3 |
| 28 | 1.1 |
| 29 | 95.0 |
| 30 | 95.0 |
| 32 | 95.0 |
| 43 | 16.0 |
| 44 | 26.0 |
| 45 | 74.0 |
| 46 | 10.0 |
| 47 | 42.0 |
| 48 | 24.0 |
| 49 | 89.0 |
| 50 | 80.0 |
| 54 | 9.0 |
| 55 | 74.0 |
| 56 | 7.1 |
| 57 | 95.0 |
| 64 | 44.0 |
| 65 | 95.0 |
| 66 | 5.2 |
| 69 | 93.0 |
| 70 | 90.0 |
| 74 | 90.0 |
| 76 | 89.0 |
| 78 | 86.0 |
| 79 | 90.0 |
| 84 | 15.0 |
| 86 | 33.0 |
| 87 | 4.7 |
| 89 | 88.0 |
| 97 | 5.0 |
| 98 | 4.7 |
| 99 | 0.2 |
| 100 | 83.1 |
| 101 | 20.8 |
| 102 | 88.0 |
| 123 | 90.0 |
| 136 | 11.0 |

### Biological Example 1—TAK1 Biochemical Assay

The usefulness of the example compounds of the invention as TAK1 inhibitors was evaluated in a biochemical ADP-Glo kinase assay. Assay kits from Promega Corporation V4089 were used according to manufacturer's instructions and adapted as outlined here. ADP-Glo Kinase Assay kit contains ADP-Glo Reagent, Kinase Detection Reagent, kinase detection buffer, ATP, and ADP. The Kinase Enzyme Systems include human recombinant TAK1-TAB1 Kinase (TAK1 (1-303) and TAB1 (437-end)), Native Swine Myelin Basic Protein (MBP) substrate, 5×kinase reaction buffer, and DTT.

Test compounds were prepared in 1:5 serial dilutions at 8 concentrations in DMSO from 10 mM stock solutions. An additional 20-fold dilution in 1× kinase reaction buffer (see below) was performed, yielding a final dilution series in kinase buffer with 5% DMSO (final concentration in assay from 20 μM to 0.26 nM, 1% DMSO). As controls, Takinib was prepared as positive control (3 μM final in assay), and DMSO without inhibitor served as negative control, at a final assay concentration of DMSO of 1%.

One μl of compound solution in 5% DMSO was added to each well in a 384 well microtiter plate (OptiPlate #6007290). Two μl TAK1-TAB1 (7.5 ng/μl) in lxkinase reaction buffer (40 nM Tris base pH 7.5, 20 mM MgCl₂, 50 μM DTT, 0.1 mg/ml BSA) was added and pre-incubated 10 min before adding 2 μl of a mixture of MBP substrate and ATP (0.25 μg/μL MBP/25 μM ATP or 0.25 μg/μL MBP/2.5 mM ATP) in 1× kinase reaction buffer (as above). The plate was subsequently incubated at room temperature for 1 hour. Then 5 μl ADP-Glo™ reagent from ADP-Glo™ kit (as above) was added and incubated for 40 min to stop the reaction and consume remaining ATP. Ten μl kinase detection substrate (dissolved in kinase detection buffer, both from ADP-Glo™ kit as above) was then added to convert ADP to ATP and detect ATP with luciferase/luciferin. The plate was incubated for 30 minutes at room temperature in dark before reading luminescence on an Envision (PerkinElmer) reader.

IC₅₀ values of compounds of the invention were determined by the non-linear regression analysis of dose response curves. The IC₅₀ results are shown in Table 2 below.

TABLE 2
Potency and efficacy of selected examples in the
TAK1 biochemical assay

| | TAK1 Mean | TAK1 Mean |
|---|---|---|
| Example | IC₅₀ (nM) @ | IC₅₀ (nM) @ |
| compound | 10 μM ATP | 1 mM ATP |
| takinib | 17 | 280 |
| 1 | 27 | n.d. |
| 2 | 3 | n.d. |
| 3 | 1 | n.d. |
| 4 | 7 | n.d. |
| 5 | 11 | 247 |
| 6 | 5 | n.d. |
| 7 | 4 | n.d. |
| 8 | 5 | n.d. |
| 9 | 1 | n.d. |
| 10 | 11 | n.d. |
| 11 | 1 | n.d. |
| 12 | 1 | n.d. |
| 13 | 1 | n.d. |
| 14 | 11 | n.d. |
| 15 | 5 | n.d. |
| 16 | 3 | n.d. |
| 17 | 4 | n.d. |
| 18 | 9 | n.d. |
| 19 | 7 | n.d. |
| 20 | 19 | n.d. |
| 21 | 7 | n.d. |
| 22 | 9 | n.d. |
| 23 | 8 | n.d. |
| 24 | 4 | n.d. |
| 25 | 2 | n.d. |
| 26 | 5 | n.d. |
| 27 | 8 | n.d. |
| 28 | 366 | n.d |
| 29 | 6 | n.d. |
| 30 | 8 | n.d. |
| 31 | 3 | 14 |
| 32 | 3 | 14 |
| 33 | 8 | n.d. |
| 34 | 2 | n.d. |
| 35 | 5 | n.d. |
| 36 | 6 | n.d. |
| 37 | 5 | n.d. |
| 38 | 4 | n.d. |
| 39 | 6 | n.d. |
| 40 | 3 | n.d. |
| 41 | 3 | n.d. |
| 42 | 7 | n.d. |
| 43 | 4 | n.d |
| 44 | 223 | n.d. |
| 45 | 4 | n.d |
| 46 | 2 | n.d. |
| 47 | 1 | n.d. |
| 48 | 1 | n.d. |
| 49 | 3 | n.d. |
| 50 | 3 | n.d |
| 51 | 136 | n.d. |
| 52 | 63 | n.d |
| 53 | 258 | n.d. |
| 54 | 8 | 25 |
| 55 | 6 | n.d. |
| 56 | 16 | n.d. |
| 57 | 3 | 6 |
| 58 | 4 | n.d |
| 59 | 4 | n.d. |
| 60 | 5 | n.d. |
| 61 | 4 | n.d. |
| 62 | 12 | n.d. |
| 63 | 8 | n.d. |
| 64 | 3 | n.d. |
| 65 | 4 | n.d. |
| 66 | 3 | n.d. |
| 67 | 5 | n.d. |
| 68 | 4 | n.d. |
| 69 | 2 | n.d |
| 70 | 5 | 6 |
| 71 | 6 | 27 |
| 72 | 7 | n.d. |
| 73 | 5 | n.d. |
| 74 | 7 | 11 |
| 75 | 11 | n.d |
| 76 | 18 | n.d. |
| 77 | 5 | n.d. |
| 78 | 8 | n.d |
| 79 | 6 | 7 |
| 80 | 5 | n.d. |
| 81 | 6 | n.d. |
| 82 | 7 | n.d. |
| 83 | 6 | n.d. |
| 84 | 5 | n.d |
| 85 | 3 | n.d. |
| 86 | 4 | 9 |
| 87 | 5 | 9 |
| 88 | 9 | n.d. |
| 89 | 6 | n.d. |
| 90 | 6 | n.d. |
| 91 | 5 | 19 |
| 92 | 6 | n.d. |
| 93 | 6 | n.d. |
| 94 | 5 | n.d |
| 95 | 6 | n.d. |
| 96 | 11 | 7 |
| 97 | 4 | n.d. |
| 98 | 5 | n.d. |
| 99 | 4 | n.d. |
| 100 | 2 | n.d. |
| 101 | 8 | n.d |
| 102 | 9 | n.d. |
| 103 | 3 | n.d. |
| 104 | 4 | n.d. |
| 105 | 6 | n.d. |
| 106 | n.d. | 6 |
| 107 | n.d. | 4 |
| 108 | n.d. | 23 |
| 109 | n.d. | 114 |
| 110 | n.d. | 26 |
| 111 | n.d. | 24 |
| 112 | n.d. | 8 |
| 113 | 5 | 5 |
| 114 | n.d. | 10 |
| 115 | n.d. | 11 |
| 116 | n.d. | 3 |
| 117 | n.d. | 4 |
| 118 | n.d. | 15 |
| 119 | 7 | 22 |
| 120 | 5 | 14 |
| 121 | 5 | 19 |
| 122 | 9 | 57 |
| 123 | 4 | 11 |
| 124 | 7 | 9 |
| 125 | 5 | 7 |
| 126 | 4 | 12 |
| 127 | 5 | 18 |
| 128 | 7 | n.d. |
| 129 | 6 | 6 |
| 130 | 7 | 27 |
| 131 | 5 | 13 |
| 132 | 10 | 10 |
| 133 | 10 | 12 |
| 134 | 10 | 12 |
| 135 | 6 | n.d. |
| 136 | 4 | n.d. |
| 137 | 9 | n.d. |
| 138 | 8 | n.d. |
| 139 | n.d. | 17 |
| 140 | n.d. | 6 |
| 141 | n.d. | 15 |
| 142 | 7 | 8 |
| 143 | n.d. | 14 |
| 144 | n.d. | 10 |
| 145 | 10 | n.d. |
| 146 | 5 | 9 |
| 147 | 5 | n.d. |

n.d. = not determined.

### Biological Example 2: Cell-Based p38 Phosphorylation Assay

The cellular activity of example compounds 2, 3 and 9 and the comparative example (i.e. takinib) for inhibiting TAK1 activity was evaluated using an in vitro functional cell assay.

Comparative example 1, takinib (N-(1-propyl-1H-benzo[d]imidazol-2-yl)isophthalamide; CAS No.:1111556-37-6), was synthesised according to the protocol described by Totzke et al. (Cell Chemical Biology, 2017, 24, 1029-1039).

THP-1 cells were incubated at 37° C. in 5% CO₂. THP-1 cells were cultured in RPMI 1640, 10% FBS, 1% Penicillin-Streptomycin.

THP-1 cells were treated with 100 nM phorbol 12-myristate 13-acetate (PMA) to induce macrophage differentiation and seeded at the density of 1×10⁶ cells/well (2 ml/well in a 6-well plate) for 72 hours. The cell culture media was then replaced with PMA-free media and incubated for further 48 hours. Test compounds were prepared as 200×final assay concentration stocks in DMSO (final concentrations in the assay were 10-0.03 μM in 0.5% DMSO, details provided below). Following THP-1 cell differentiation, cells were treated with different concentrations of the control compound NG25 (CAS No.:1315355-93-1, Tan L, et al. J Med Chem. 2015, 58(1), 183-96) or test compounds (10 μl/well) for 30 minutes followed by 200 ng/ml LPS for 30 minutes.

Test items were kept as 10 mM stock solutions in 100% DMSO. The serial dilution of compounds was prepared as 200× serial dilutions of desired final concentration in culture medium to reach 0.5% DMSO working solution (see Table 3 below).

TABLE 3
Serial dilution of example and comparative compounds

| Final Conc | | |
|---|---|---|
| to assay | | |
| μM | μM | 10 + 40 |
| 10 | 2000 | 15 μl stock + 35 μl DMSO |
| 3 | 600 | 15 μl 3 mM stock + 30 μl DMSO |
| 1 | 200 | 15 μl 0.3 mM stock + 30 μl DMSO |
| 0.3 | 60 | 15 μl 0.1 mM stock + 35 μl DMSO |
| 0.1 | 20 | 15 μl stock + 35 μl DMSO |
| 0.03 | 6 | 15 μl stock + 35 μl DMSO |
| 0 | 0 | |
| Blank | Blank | |

Dilution series of compounds from 10 μM final highest concentration to 0.03 μM final low concentration were first made in DMSO (see Table 3), 10 μl of each dilution was mixed into 2 ml of cell cultures (0.5% DMSO per well).

10 μl of DMSO was added to the control. All plates were incubated at 37° C., 5% CO2 for 30 minutes and LPS was added to a final concentration of 200 ng/ml.

For Western blot analysis, cells were lysed (50 mM Tris, 150 mM NaCl, 1 mM EDTA, 1% Triton-X100, 1 mM DTT, complete protease and PhosStop phosphatase inhibitor) after indicated treatment and run on NuPAGE Bis-Tris gel 4-12% gradient (Invitrogen). Following transfer to PVDF membrane and blocking in 5% BSA in PBST, membranes were incubated with primary antibody overnight (specific for phosphorylated p38; Cell Signaling, 1:1,000 dilution). After incubation with species-specific secondary antibodies for 1 hour at room temperature, the membranes were visualized with the ECL SuperSignal™ West Pico PLUS Chemiluminescent Substrate (Thermo Scientific). The images were captured with iBright Western Blot Imaging Systems (Invitrogen) and quantified with ImageJ software (Java-based image processing program developed at the National Institutes of Health and the Laboratory for Optical and Computational Instrumentation). Anti-β-actin antibodies were used as the loading control for whole-cell extracts in all samples (anti-β actin; Sigma-Aldrich, 1:5000 dilution, 1 hour incubation).

For immunoblotting, cells were lysed directly in six-well plates by incubating for 30 min at 4° C. in 60 μl Triton lysis buffer. The lysates were centrifuged for 15 minutes at 13,000×g at 4° C. Equal amount of protein from each supernatants were resuspended with 5× sample buffer. Samples were boiled for 10 minutes and then resolved by 4-12% SDS-PAGE (100 V). Proteins were transferred from gel to PVDF membranes (0.45 m pore size) following the recommended setting (90 minutes, 85 V). Blots were blocked for 1 hour in 5% BSA diluted with PBS-T. Blots were incubated with the following antibodies overnight at 4° C. with primary antibodies specific for phosphorylated p38 (Cell Signaling, 1:1,000 dilution) and anti-B actin (Sigma-Aldrich, 1:5000 dilution, 1 hour incubation). Blots were washed three times, for 5 minutes each, and incubated with species-specific secondary antibodies [Goat anti-rabbit IgG (H+L) (Invitrogen, 1:50,000 dilution) or secondary antibodies [Goat anti-mouse IgG (H+L) (Invitrogen, 1:50,000 dilution) for 1 hour at room temperature. Blots were washed three times, for 5 minutes each, and developed by chemiluminescence (SuperSignal™ West Pico PLUS Chemiluminescent Substrate).

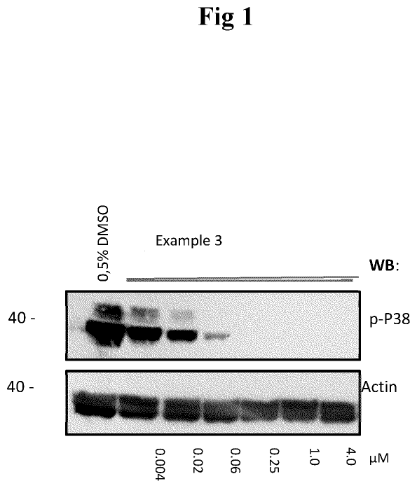

The activity of Examples 2, 3 and 9 and a comparative example 1 was also determined by measuring the amount of phosphorylated p38. Since p38 is a target of TAK1, phosphorylated p38 is a measure of TAK1 activity. A Western blot obtained in this way using Example 3 is shown in FIG. 1.

IC₅₀ values of test compounds were determined by the non-linear regression analysis of dose response curves. The results are shown in Table 4 below.

As can be seen from Table 4, Example 3 had a low IC₅₀ value, Furthermore, the potency and the efficacy of Examples 2, 3 and 9 were greater than the potency and the efficacy Comparative Example 1, which is a known tak1 inhibitor.

TABLE 4
Potency and efficacy of selected example compounds and
comparative example 1 (takanib) in a phospho-p38 cell assay

| Example compounds | IC₅₀ (nM) |
|---|---|
| takinib | inactive |
| 2 | 200 |
| 3 | 4.5 |
| 9 | 600 |

1. A compound according to formula (I) 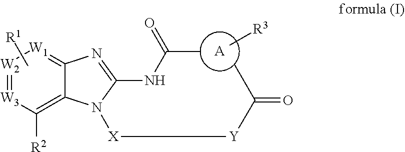
or a tautomer thereof,
wherein,
A is a 6 membered aromatic ring or 5 to 6 membered heteroaromatic ring comprising 1 or 2 atoms selected from the group consisting of N, S and O;
W₁, W₂, W₃ are independently selected from CH and N;
X is linear C₂₋₈alkylene, optionally substituted with one or more groups independently selected from halogen; OH; C₃₋₆cycloalkyl; C₁₋₆alkyl optionally substituted with one or more halogen or OH; and two groups attached to the same carbon atom in the linear C₂₋₈alkylene which together with the atom in the linear C₂₋₈alkylene form a 3- or 4-membered cycloalkyl group; and/or wherein optionally one or more carbon atoms in the alkylene is replaced by a group independently selected from O, NH and S;
Y is NH or O;
R¹ and R² are independently selected from H; C₁₋₆alkyl; C₂₋₆alkenyl; C₂₋₆alkynyl; OC₁₋₆ alkyl; OC₂₋₆alkenyl; OC₂₋₆alkynyl; —R^{a}, C(O)R^{a}; C(O)OR^{a}; OCR^{a}; OC(O)(R^{a}); —C₁₋₆ alkylene-R^{a}; —C(O)C₁₋₆alkylene-R^{a}; —C(O)OC₁₋₆alkylene-R^{a}; —OC₁₋₆alkylene-R^{a}; OC(O)C₁₋₆alkylene-R^{a}; and —R^{b}, wherein said alkyl, alkenyl, or alkynyl is optionally substituted with one or more R^{b} groups;
R³ is H, halogen, OH, C₁₋₆alkyl or OC₁₋₆alkyl, wherein said alkyl is optionally substituted with one or more halogen;
wherein,
R^{a} is selected from C₃₋₈cycloalkyl or 3 to 12 membered heterocycle comprising one, two or three atoms selected from the group consisting of N, S and O; said cycloalkyl or heterocycle optionally being bridged by C₁₋₄alkylene and/or substituted by one or more groups independently selected from C₁₋₄alkyl; 3 to 6 membered heterocycle comprising one, two or three atoms selected from the group consisting of N, S and O; C₃₋₆cycloalkyl; OC₁₋₄alkyl; O-(3 to 6 membered heterocycle comprising one, two or three atoms selected from the group consisting of N, S and O); OC₃₋₆cycloalkyl; C(O)C₁₋₄alkyl; C(O)OC₁₋₄alkyl; OC(O)C₁₋₄alkyl; C(O)NH₂; and —C₁₋₄alkylene-OC₁₋₄alkyl; wherein said alkyl, alkylene, heterocycle or cycloalkyl at R^{a} is optionally substituted by one or more groups independently selected from halogen; OH; O; SH; NH₂; NHC₁₋₄alkyl; or N(C₁₋₄alkyl)₂; and
Each R^{b} is independently selected from C(O)R^{c}; C(O)OR^{c}; OC(O)R^{c}; SR^{c}; SO₂R^{c}; NO₂; CN; NR^{c}R^{d}; N(R^{c})SO₂C₁₋₃alkyl; N(R^{c})C(O)R^{d}; C(O)NR^{c}R^{d}; SO₂NR^{c}R^{d}, halogen; SH; NH₂; and OH; said alkyl optionally being substituted with one or more groups independently selected from halogen, SH, NH₂ and OH and/or wherein optionally one or more carbon atoms in the alkyl is replaced by a group independently selected from O, NH and S; and
R^{c} and R^{d} are independently selected from H; C₁₋₆alkyl; C₂₋₆alkenyl; C₂₋₆alkynyl; —C₁₋₄alkylene-OC₁₋₄alkyl; and C₃₋₈cycloalkyl; said alkyl, alkylene, alkenyl, alkynyl or cycloalkyl optionally being substituted with one or more groups selected from halogen, SH, NH₂, OH and ═O, and/or wherein optionally one or more carbon atoms in the alkyl is replaced by an atom independently selected from O, N and S;
or a pharmaceutically acceptable salt or solvate thereof.

2. A compound as claimed in claim 1, wherein X is a linear C₄₋₈alkylene, preferably X is a linear C₅₋₆alkylene, more preferably X is a linear C₅alkylene.

3. A compound as claimed in claim 1 or 2, wherein one or two carbon atoms in the alkylene of X is replaced by a group independently selected from O, NH and S, preferably one carbon atom in the alkylene of X is replaced by O or NH, for example, X may be —(CH₂)₂—O—(CH₂)₂—.

4. A compound as claimed in any one of claims 1 to 3, wherein X is substituted with one, two or three groups independently selected from halogen (for example, F, Cl, Br, I), OH and C₁₋₆alkyl, preferably X is substituted with one methyl group, for example X may be —CH(CH₃)(CH₂)₄—.

5. A compound as claimed in any one of claims 1 to 4, wherein R¹ is H.

6. A compound as claimed in any one of claims 1 to 5, wherein R² is H.

7. A compound as claimed in any one of claims 1 to 4, wherein
R¹ is H; and
R² is —R^{a}, C(O)R^{a}; C(O)OR^{a}; OC(O)(R^{a}); -methylene-R^{a}; or —R^{b}.

8. A compound as claimed in any one of claims 1 to 4, wherein
R¹ is methyl; —R^{a}, C(O)R^{a}; C(O)OR^{a}; OC(O)(R^{a}); -methylene-R^{a}; or —R^{b}; and
R² is H.

9. A compound as claimed in any one of claims 1 to 4, wherein
R¹ and R² are independently selected from methyl; —R^{a}, C(O)R^{a}; C(O)OR^{a}; OC(O)(R^{a}); -methylene-R^{a}; or —R^{b}.

10. A compound as claimed in any one of claims 1 to 9, wherein:
R^{a} is a heterocycle selected from piperazinyl, piperidinyl, morpholinyl, pyrrolidinyl, oxopiperidinyl, homopiperazinyl, pyridinyl, oxodihydropyridinyl, pyrrolidinyl, azetidineyl, triazolyl, oxadiazolenyl, imidazoleyl, oxazolidinoneyl or 9 to 11 membered spirocyclic heterocycle comprising one or two atoms selected from the group consisting of N and O, said heterocycle or spirocyclic heterocycle optionally being bridged by C₁₋₄alkylene and/or substituted with one group independently selected from methyl, ethyl, 3 to 4 membered heterocycle comprising one atoms selected from the group consisting of N, S and O; C₃₋₆cycloalkyl; O-(3 to 6 membered heterocycle comprising one, two or three atoms selected from the group consisting of N, S and O); OC₁₋₄alkyl; C(O)C₁₋₄alkyl; C(O)OC₁₋₄alkyl; OC(O)C₁₋₄alkyl; C(O)NH₂; or —C₁₋₄alkylene-OC₁₋₄alkyl; wherein said alkyl, alkylene, heterocycle, spirocyclic heterocycle (for example non-aromatic spirocyclic heterocycle) or cycloalkyl is optionally substituted by one or more groups independently selected from the group consisting of F, Cl and OH.

11. A compound as claimed in any one of claims 1 to 10, wherein R^{a} is selected from the group consisting of:

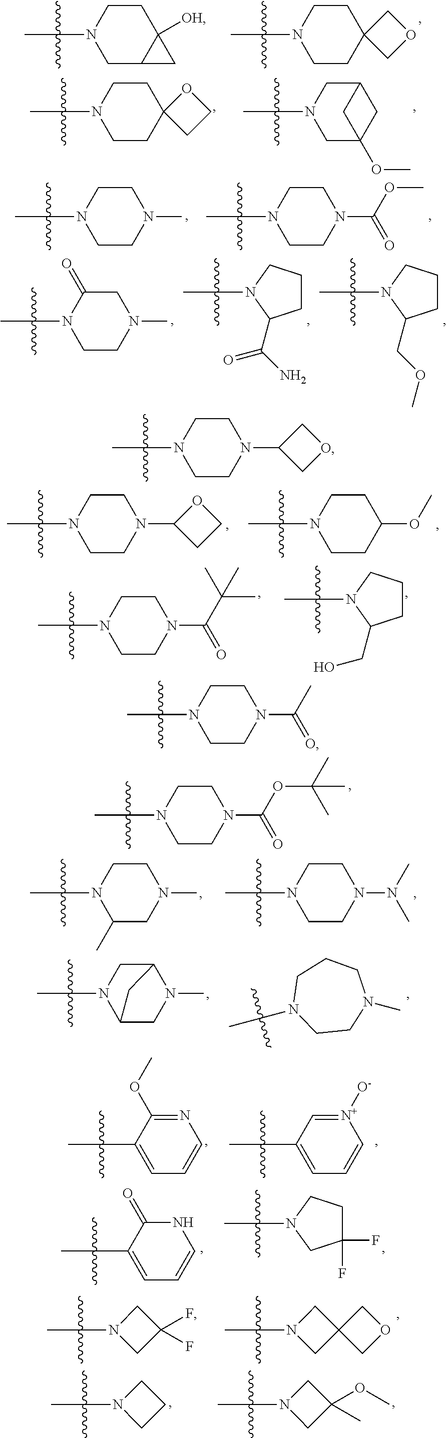

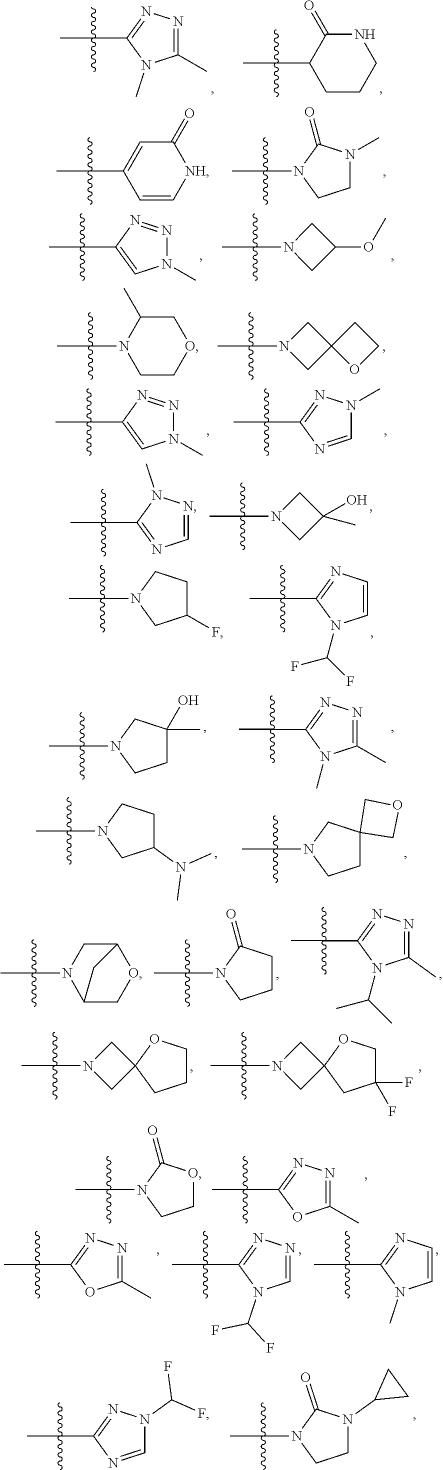

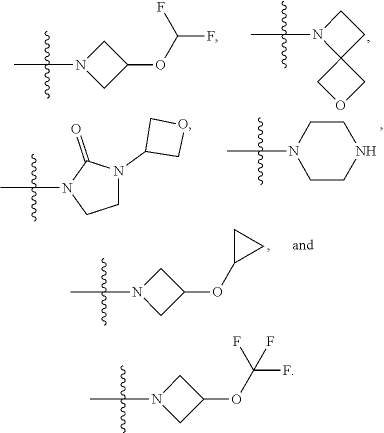

12. A compound as claimed in any one of claims 1 to 11, wherein R^{b} is independently selected from C(O)R^{c}; C(O)OR^{c}; OC(O)R^{c}; SR^{c}; SO₂R^{c}; NO₂; CN; NR^{c}R^{d}; N(R^{c})SO₂C₁₋₃alkyl; N(R^{c})C(O)R^{d}; C(O)NR^{c}R^{d}; F; Cl; SH; NH₂; or OH; said alkyl optionally being substituted with one or two groups independently selected from F, Cl, SH, NH₂ and OH; preferably, R^{b} is C(O)R^{c} or C(O)NR^{c}R^{d}.

13. A compound as claimed in any one of claims 1 to 12, wherein R^{c} and R^{d} are independently selected from H, methyl, ethyl, propyl and —C₁₋₂alkylene-OCH₃, preferably, R^{c} and R^{d} are each methyl.

14. A compound as claimed in any one of claims 1 to 13, wherein R³ is H, F, Cl, Br, I, C₁₋₄alkyl or OC₁₋₄alkyl, preferably R³ is H or F, more preferably R³ is H.

15. A compound as claimed in any one of claims 1 to 14, wherein A is phenyl or thienyl, preferably A is phenyl.

16. A compound as claimed in any one of claims 1 to 15, wherein W₁ and W₂ are each CH and W₃ is CH or N, preferably W₁, W₂, W₃ are each CH.

17. A compound as claimed in any one of claims 1 to 16, wherein 1 to 6 hydrogen atoms are each replaced by a deuterium atom, wherein each deuterium has an abundance level of at least 1 mol %, 5 mol %, 10 mol %, 50 mol %, 90 mol % or 98 mol % deuterium.

18. A compound as claimed in claim 1, wherein the compound is selected from the group consisting of:
1¹H-2,6-diaza-1(2,1)-benzo[d]imidazola-4(1,3)-benzenacyclododecaphane-3,5-dione (Example 1);
1¹H-2,6-diaza-1(2,1)-benzo[d]imidazola-4(1,3)-benzenacycloundecaphane-3,5-dione (Example 2);
1⁵-((4-methylpiperazin-1-yl)methyl)-1 ¹H-2,6-diaza-1(2,1)-benzo[d]imidazola-4(1,3)-benzenacycloundecaphane-3,5-dione (Example 3);
1⁵-methyl-1¹H-2,6-diaza-1(2,1)-benzo[d]imidazola-4(1,3)-benzenacycloundecaphane-3,5-dione (Example 4);
1¹H-9-oxa-2,6-diaza-1(2,1)-benzo[d]imidazola-4(1,3)-benzenacycloundecaphane-3,5-dione (Example 5);
11-methyl-1¹H-2,6-diaza-1(2,1)-benzo[d]imidazola-4(1,3)-benzenacycloundecaphane-3,5-dione (Example 6);
11-methyl-3,5-dioxo-1¹H-2,6-diaza-1(2,1)-benzo[d]imidazola-4(1,3)-benzenacycloundecaphane-1⁷-carboxylate (Example 7);
11-methyl-1⁷-(4-methylpiperazine-1-carbonyl)-1¹H-2,6-diaza-1(2,1)-benzo[d]imidazola-4(1,3)-benzenacycloundecaphane-3,5-dione (Example 8);
tert-butyl 4-(11-methyl-3,5-dioxo-1H-2,6-diaza-1(2,1)-benzo[d]imidazola-4(1,3)-benzenacycloundecaphane-1⁷-carbonyl)piperazine-1-carboxylate (Example 9);
1⁷-(4-methylpiperazine-1-carbonyl)-1H-2,6-diaza-1(2,1)-benzo[d]imidazola-4(1,3)-benzenacycloundecaphane-3,5-dione (Example 10);
1⁵-(4-methylpiperazine-1-carbonyl)-1¹H-2,6-diaza-1(2,1)-benzo[d]imidazola-4(1,3)-benzenacycloundecaphane-3,5-dione (Example 11);
1⁵-(4-(oxetan-3-yl)piperazine-1-carbonyl)-1¹H-2,6-diaza-1(2,1)-benzo[d]imidazola-4(1,3)-benzenacycloundecaphane-3,5-dione (Example 12);
tert-butyl 4-(3,5-dioxo-1¹H-2,6-diaza-1(2,1)-benzo[d]imidazola-4(1,3)-benzenacycloundecaphane-1⁵-carbonyl)piperazine-1-carboxylate (Example 13);
tert-butyl 4-(3,5-dioxo-1¹H-2,6-diaza-1(2,1)-benzo[d]imidazola-4(1,3)-benzenacycloundecaphane-1⁷-carbonyl)piperazine-1-carboxylate (Example 14);
11-methyl-1⁷-(piperazine-1-carbonyl)-1¹H-2,6-diaza-1(2,1)-benzo[d]imidazola-4(1,3)-benzenacycloundecaphane-3,5-dione (Example 15);
1⁵-(piperazine-1-carbonyl)-1¹H-2,6-diaza-1(2,1)-benzo[d]imidazola-4(1,3)-benzenacycloundecaphane-3,5-dione (Example 16);
1⁷-(piperazine-1-carbonyl)-1¹H-2,6-diaza-1(2,1)-benzo[d]imidazola-4(1,3)-benzenacycloundecaphane-3,5-dione (Example 17);
(E)-3,5-dioxo-1²,1³-dihydro-1¹H-2,6-diaza-1(2,1)-benzo[d]imidazola-4(1,3)-benzenacycloundecaphane-1⁷-carboxylic acid (Example 18);
(E)-1⁷-(4-(oxetan-3-yl)piperazine-1-carbonyl)-1²,1³-dihydro-1¹H-2,6-diaza-1(2,1)-benzo[d]imidazola-4(1,3)-benzenacycloundecaphane-3,5-dione (Example 19);
(R,E)-1⁷-(2,4-dimethylpiperazine-1-carbonyl)-1²,1³-dihydro-1¹H-2,6-diaza-1(2,1)-benzo[d]imidazola-4(1,3)-benzenacycloundecaphane-3,5-dione (Example 20);
(S,E)-1⁷-(3-(dimethylamino)pyrrolidine-1-carbonyl)-1²,1³-dihydro-1¹H-2,6-diaza-1(2,1)-benzo[d]imidazola-4(1,3)-benzenacycloundecaphane-3,5-dione (Example 21);
(E)-1⁷-((1S,4S)-5-methyl-2,5-diazabicyclo[2.2.1]heptane-2-carbonyl)-1²,1³-dihydro-1¹H-2,6-diaza-1(2,1)-benzo[d]imidazola-4(1,3)-benzenacycloundecaphane-3,5-dione (Example 22);
Example 23: (R,E)-1⁷-(3-(dimethylamino)pyrrolidine-1-carbonyl)-1²,1³-dihydro-1¹H-2,6-diaza-1(2,1)-benzo[d]imidazola-4(1,3)-benzenacycloundecaphane-3,5-dione (Example 23);
(S,E)-1⁷-(2,4-dimethylpiperazine-1-carbonyl)-1²,1³-dihydro-1¹H-2,6-diaza-1(2,1)-benzo[d]imidazola-4(1,3)-benzenacycloundecaphane-3,5-dione (Example 24);
(E)-1⁷-((1R,4R)-5-methyl-2,5-diazabicyclo[2.2.1]heptane-2-carbonyl)-1²,1³-dihydro-1¹H-2,6-diaza-1(2,1)-benzo[d]imidazola-4(1,3)-benzenacycloundecaphane-3,5-dione (Example 25);
(E)-1⁷-(4-methyl-1,4-diazepane-1-carbonyl)-1²,1³-dihydro-1¹H-2,6-diaza-1(2,1)-benzo[d]imidazola-4(1,3)-benzenacycloundecaphane-3,5-dione (Example 26);
(E)-N,N-dimethyl-1⁵-((4-methylpiperazin-1-yl)methyl)-3,5-dioxo-1²,1³-dihydro-1¹H-2,6-diaza-1(2,1)-benzo[d]imidazola-4(1,3)-benzenacycloundecaphane-1⁷-carboxamide (Example 27);
(E)-1⁷-(2-methoxypyridin-3-yl)-1²,1³-dihydro-1¹H-2,6-diaza-1(2,1)-benzo[d]imidazola-4(1,3)-benzenacycloundecaphane-3,5-dione (Example 28);
(R, E)-11-methyl-1⁷-((1R,4R)-5-methyl-2,5-diazabicyclo[2.2.1]heptane-2-carbonyl)-1²,1³-dihydro-1¹H-2,6-diaza-1(2,1)-benzo[d]imidazola-4(1,3)-benzenacycloundecaphane-3,5-dione (Example 29);
(S, E)-11-methyl-1⁷-((1R,4R)-5-methyl-2,5-diazabicyclo[2.2.1]heptane-2-carbonyl)-1²,1³-dihydro-1¹H-2,6-diaza-1(2,1)-benzo[d]imidazola-4(1,3)-benzenacycloundecaphane-3,5-dione (Example 30);
(E)-N,N,11-trimethyl-3,5-dioxo-1²,1³-dihydro-1¹H-2,6-diaza-1(2,1)-benzo[d]imidazola-4(1,3)-benzenacycloundecaphane-1⁷-carboxamide (Example 31 and 32);
((E)-3-(11-methyl-3,5-dioxo-1²,1³-dihydro-1¹H-2,6-diaza-1(2,1)-benzo[d]imidazola-4(1,3)-benzenacycloundecaphane-1⁷-yl)pyridine 1-oxide (Example 33);
(E)-N,11-dimethyl-3,5-dioxo-1²,1³-dihydro-1¹H-2,6-diaza-1(2,1)-benzo[d]imidazola-4(1,3)-benzenacycloundecaphane-1⁷-carboxamide (Example 34);
(E)-11-methyl-1⁷-(2-oxo-1,2-dihydropyridin-3-yl)-1²,1³-dihydro-1¹H-2,6-diaza-1(2,1)-benzo[d]imidazola-4(1,3)-benzenacycloundecaphane-3,5-dione (Example 35 and 36);
(E)-1⁷-(3,3-difluoropyrrolidine-1-carbonyl)-11-methyl-1²,1³-dihydro-1¹H-2,6-diaza-1(2,1)-benzo[d]imidazola-4(1,3)-benzenacycloundecaphane-3,5-dione (Example 37);
methyl (E)-N-methyl-N-(11-methyl-3,5-dioxo-1²,1³-dihydro-1¹H-2,6-diaza-1(2,1)-benzo[d]imidazola-4(1,3)-benzenacycloundecaphane-1⁷-carbonyl)glycinate (Example 38);
(E)-N-cyclobutyl-N,11-dimethyl-3,5-dioxo-1²,1³-dihydro-1¹H-2,6-diaza-1(2,1)-benzo[d]imidazola-4(1,3)-benzenacycloundecaphane-1⁷-carboxamide (Example 39);
(E)-1⁷-(3,3-difluoroazetidine-1-carbonyl)-11-methyl-1²,1³-dihydro-1¹H-2,6-diaza-1(2,1)-benzo[d]imidazola-4(1,3)-benzenacycloundecaphane-3,5-dione (Example 40);
(E)-N-(3-hydroxycyclobutyl)-N,11-dimethyl-3,5-dioxo-1²,1³-dihydro-1¹H-2,6-diaza-1(2,1)-benzo[d]imidazola-4(1,3)-benzenacycloundecaphane-1⁷-carboxamide (Example 41);
(E)-11-methyl-1⁷-(2-oxa-6-azaspiro[3.3]heptane-6-carbonyl)-1²,1³-dihydro-1¹H-2,6-diaza-1(2,1)-benzo[d]imidazola-4(1,3)-benzenacycloundecaphane-3,5-dione (Example 42);
(E)-1⁷-(azetidine-1-carbonyl)-11-methyl-1²,1³-dihydro-1¹H-2,6-diaza-1(2,1)-benzo[d]imidazola-4(1,3)-benzenacycloundecaphane-3,5-dione (Example 43);
(S,E)-N,N,7-trimethyl-3,5-dioxo-1²,1³-dihydro-1¹H-2,6-diaza-1(2,1)-benzo[d]imidazola-4(1,3)-benzenacycloundecaphane-1⁷-carboxamide (Example 44);
(E)-N-(11-methyl-3,5-dioxo-1²,1³-dihydro-1¹H-2,6-diaza-1(2,1)-benzo[d]imidazola-4(1,3)-benzenacycloundecaphane-1⁷-yl)acetamide (Example 45);
(R,E)-N,N,11-trimethyl-3,5-dioxo-1²,1³-dihydro-1¹H-2,6-diaza-1(2,1)-benzo[d]imidazola-4(1,3)-benzenacycloundecaphane-1⁷-sulfonamide (Example 46);
(E)-N-isopropyl-N,11-dimethyl-3,5-dioxo-1²,1³-dihydro-1¹H-2,6-diaza-1(2,1)-benzo[d]imidazola-4(1,3)-benzenacycloundecaphane-1⁷-carboxamide (Example 47 and 48);
(E)-1⁷-(3-methoxy-3-methylazetidine-1-carbonyl)-11-methyl-1²,1³-dihydro-1¹H-2,6-diaza-1(2,1)-benzo[d]imidazola-4(1,3)-benzenacycloundecaphane-3,5-dione (Example 49 and 50);
(E)-N,N,10-trimethyl-3,5-dioxo-1²,1³-dihydro-1¹H-2,6-diaza-1(2,1)-benzo[d]imidazola-4(1,3)-benzenacycloundecaphane-1⁷-carboxamide (Example 51 and 52);
(S,E)-1⁶-chloro-N,N,11-trimethyl-3,5-dioxo-1²,1³-dihydro-1¹H-2,6-diaza-1(2,1)-benzo[d]imidazola-4(1,3)-benzenacycloundecaphane-1⁷-carboxamide (Example 53 and 54);
(R,E)-1⁶-chloro-N,N,11-trimethyl-3,5-dioxo-1²,1³-dihydro-1¹H-2,6-diaza-1(2,1)-benzo[d]imidazola-4(1,3)-benzenacycloundecaphane-1⁷-carboxamide (Example 55 and 56);
(R,E)-1⁷-(4,5-dimethyl-4H-1,2,4-triazol-3-yl)-11-methyl-1²,1³-dihydro-1¹H-2,6-diaza-1(2,1)-benzo[d]imidazola-4(1,3)-benzenacycloundecaphane-3,5-dione (Example 57);
(E)-11-methyl-1⁷-(2-oxopiperidin-3-yl)-1²,1³-dihydro-1¹H-2,6-diaza-1(2,1)-benzo[d]imidazola-4(1,3)-benzenacycloundecaphane-3,5-dione (Examples 58 to 61);
(E)-11-methyl-1⁷-(2-oxo-1,2-dihydropyridin-4-yl)-1²,1³-dihydro-1¹H-2,6-diaza-1(2,1)-benzo[d]imidazola-4(1,3)-benzenacycloundecaphane-3,5-dione (Example 62 and 63);
(R,E)-11-methyl-N,N-bis(methyl-d₃)-3,5-dioxo-1²,1³-dihydro-1¹H-2,6-diaza-1(2,1)-benzo[d]imidazola-4(1,3)-benzenacycloundecaphane-1⁷-carboxamide (Example 64);
(R,E)-11-methyl-1⁷-(3-methyl-2-oxoimidazolidin-1-yl)-1²,1³-dihydro-1¹H-2,6-diaza-1(2,1)-benzo[d]imidazola-4(1,3)-benzenacycloundecaphane-3,5-dione (Example 65);
(R,E)-11-methyl-1⁷-(1-methyl-1H-1,2,3-triazol-4-yl)-1²,1³-dihydro-1¹H-2,6-diaza-1(2,1)-benzo[d]imidazola-4(1,3)-benzenacycloundecaphane-3,5-dione (Example 66);
(E)-11-ethyl-N,N-dimethyl-3,5-dioxo-1²,1³-dihydro-1¹H-2,6-diaza-1(2,1)-benzo[d]imidazola-4(1,3)-benzenacycloundecaphane-1⁷-carboxamide (Example 67 and 68);
(R,E)-1⁵-(2-hydroxypropan-2-yl)-N,N,11-trimethyl-3,5-dioxo-1²,1³-dihydro-1¹H-2,6-diaza-1(2,1)-benzo[d]imidazola-4(1,3)-benzenacycloundecaphane-1⁷-carboxamide (Example 69);
((E)-N-(2-fluoroethyl)-N,11-dimethyl-3,5-dioxo-1²,1³-dihydro-1¹H-2,6-diaza-1(2,1)-benzo[d]imidazola-4(1,3)-benzenacycloundecaphane-1⁷-carboxamide (Example 70 and 71);
(E)-N-(2,2-difluoroethyl)-N,11-dimethyl-3,5-dioxo-1²,1³-dihydro-1¹H-2,6-diaza-1(2,1)-benzo[d]imidazola-4(1,3)-benzenacycloundecaphane-1⁷-carboxamide (Example 72 and 73);
(S,E)-11-methyl-1⁷-(3-methyl-2-oxoimidazolidin-1-yl)-1²,1³-dihydro-1¹H-2,6-diaza-1(2,1)-benzo[d]imidazola-4(1,3)-benzenacycloundecaphane-3,5-dione (Example 74);
(E)-11-methyl-1⁷-((R)-3-methylmorpholine-4-carbonyl)-1²,1³-dihydro-1¹H-2,6-diaza-1(2,1)-benzo[d]imidazola-4(1,3)-benzenacycloundecaphane-3,5-dione (Example 75 and 76);
(E)-11-methyl-1⁷-((S)-3-methylmorpholine-4-carbonyl)-1²,1³-dihydro-1¹H-2,6-diaza-1(2,1)-benzo[d]imidazola-4(1,3)-benzenacycloundecaphane-3,5-dione (Example 77 and 78);
(E)-11-methyl-1⁷-(1-oxa-6-azaspiro[3.3]heptane-6-carbonyl)-1²,1³-dihydro-1¹H-2,6-diaza-1(2,1)-benzo[d]imidazola-4(1,3)-benzenacycloundecaphane-3,5-dione (Example 79 and 80);
(E)-1⁷-(3-methoxyazetidine-1-carbonyl)-11-methyl-1²,1³-dihydro-1¹H-2,6-diaza-1(2,1)-benzo[d]imidazola-4(1,3)-benzenacycloundecaphane-3,5-dione (Example 81 and 82);
(R,E)-11-methyl-1⁷-(1-methyl-1H-1,2,4-triazol-3-yl)-1²,1³-dihydro-1¹H-2,6-diaza-1(2,1)-benzo[d]imidazola-4(1,3)-benzenacycloundecaphane-3,5-dione (Example 83 and 84)
(R,E)-11-methyl-1⁷-(1-methyl-1H-1,2,4-triazol-5-yl)-1²,1³-dihydro-1¹H-2,6-diaza-1(2,1)-benzo[d]imidazola-4(1,3)-benzenacycloundecaphane-3,5-dione (Example 85 and 86);
(S,E)-11-methyl-1⁷-(1-methyl-1H-1,2,3-triazol-4-yl)-1²,1³-dihydro-1¹H-2,6-diaza-1(2,1)-benzo[d]imidazola-4(1,3)-benzenacycloundecaphane-3,5-dione (Example 87);
(E)-1⁷-((S)-3-fluoropyrrolidine-1-carbonyl)-11-methyl-1²,1³-dihydro-1¹H-2,6-diaza-1(2,1)-benzo[d]imidazola-4(1,3)-benzenacycloundecaphane-3,5-dione (Example 88 and 89);
(E)-11-methyl-1⁷-(1-methyl-1H-imidazol-2-yl)-1²,1³-dihydro-1¹H-2,6-diaza-1(2,1)-benzo[d]imidazola-4(1,3)-benzenacycloundecaphane-3,5-dione (Example 90 and 91);
(E)-1⁷-((R)-3-fluoropyrrolidine-1-carbonyl)-11-methyl-1²,1³-dihydro-1¹H-2,6-diaza-1(2,1)-benzo[d]imidazola-4(1,3)-benzenacycloundecaphane-3,5-dione (Example 92 and 93);
(R,E)-1⁷-(3-hydroxy-3-methylazetidine-1-carbonyl)-11-methyl-1²,1³-dihydro-1¹H-2,6-diaza-1(2,1)-benzo[d]imidazola-4(1,3)-benzenacycloundecaphane-3,5-dione (Example 94);
(S,E)-1⁷-(3-hydroxy-3-methylazetidine-1-carbonyl)-11-methyl-1²,1³-dihydro-1¹H-2,6-diaza-1(2,1)-benzo[d]imidazola-4(1,3)-benzenacycloundecaphane-3,5-dione (Example 95);
(S,E)-1⁷-(4,5-dimethyl-4H-1,2,4-triazol-3-yl)-11-methyl-1²,1³-dihydro-1¹H-2,6-diaza-1(2,1)-benzo[d]imidazola-4(1,3)-benzenacycloundecaphane-3,5-dione (Example 96);
(E)-1⁶-(4-methylpiperazine-1-carbonyl)-1²,1³-dihydro-1¹H-2,6-diaza-1(2,1)-benzo[d]imidazola-4(1,3)-benzenacycloundecaphane-3,5-dione (Example 97);
(E)-1⁶-(4-(oxetan-3-yl)piperazine-1-carbonyl)-1²,1³-dihydro-1¹H-2,6-diaza-1(2,1)-benzo[d]imidazola-4(1,3)-benzenacycloundecaphane-3,5-dione (Example 98);
(E)-1⁵-fluoro-1²,1³-dihydro-1¹H-2,6-diaza-1(2,1)-benzo[d]imidazola-4(1,3)-benzenacycloundecaphane-3,5-dione (Example 99);
(E)-1⁶-(piperazine-1-carbonyl)-1²,1³-dihydro-1¹H-2,6-diaza-1(2,1)-benzo[d]imidazola-4(1,3)-benzenacycloundecaphane-3,5-dione (Example 100);
(E)-1⁶-((4-methylpiperazin-1-yl)methyl)-1²,1³-dihydro-1¹H-2,6-diaza-1(2,1)-benzo[d]imidazola-4(1,3)-benzenacycloundecaphane-3,5-dione (Example 101);
(E)-1⁵-((4-methylpiperazin-1-yl)methyl)-1²,1³-dihydro-1¹H-2,6-diaza-1(2,1)-benzo[d]imidazola-4(2,6)-pyridinacycloundecaphane-3,5-dione (Example 102);
(E)-1⁵-((4-methylpiperazin-1-yl)methyl)-1²,1³-dihydro-1¹H-2,6-diaza-1(2,1)-benzo[d]imidazola-4(2,6)-pyridinacycloundecaphane-3,5-dione (Example 103);
(1²E,4²Z)-1⁵-((4-methylpiperazin-1-yl)methyl)-1²,1³-dihydro-1¹H-2,6-diaza-1(2,1)-benzo[d]imidazola-4(4,2)-thiophenacycloundecaphane-3,5-dione (Example 104);
(E)-4⁶-fluoro-1⁵-((4-methylpiperazin-1-yl)methyl)-1²,1³-dihydro-1¹H-2,6-diaza-1(2,1)-benzo[d]imidazola-4(1,3)-benzenacycloundecaphane-3,5-dione (Example 105);
(S,E)-1⁷-((R)-3-hydroxy-3-methylpyrrolidine-1-carbonyl)-11-methyl-1²,1³-dihydro-1¹H-2,6-diaza-1(2,1)-benzo[d]imidazola-4(1,3)-benzenacycloundecaphane-3,5-dione (Example 106);
(S,E)-1⁷-(3-cyclopropoxyazetidine-1-carbonyl)-11-methyl-1²,1³-dihydro-1¹H-2,6-diaza-1(2,1)-benzo[d]imidazola-4(1,3)-benzenacycloundecaphane-3,5-dione (Example 107);
(1²Z,4²E)-N,N,5-trimethyl-2,11-dioxo-4²,4³-dihydro-4¹H-3,10-diaza-1(4,2)-thiazola-4(2,1)-benzo[d]imidazolacycloundecaphane-4⁷-carboxamide (Example 108 and 109);
(S,E)-11-methyl-1⁷-(3-(trifluoromethoxy)azetidine-1-carbonyl)-1²,1³-dihydro-11H-2,6-diaza-1(2,1)-benzo[d]imidazola-4(1,3)-benzenacycloundecaphane-3,5-dione (Example 110);
(S,E)-1⁷-((1S,4S)-2-oxa-5-azabicyclo[2.2.1]heptane-5-carbonyl)-11-methyl-1²,1³-dihydro-1¹H-2,6-diaza-1(2,1)-benzo[d]imidazola-4(1,3)-benzenacycloundecaphane-3,5-dione (Example 111);
(S,E)-1⁷-((1R,4R)-2-oxa-5-azabicyclo[2.2.1]heptane-5-carbonyl)-11-methyl-1²,1³-dihydro-1¹H-2,6-diaza-1(2,1)-benzo[d]imidazola-4(1,3)-benzenacycloundecaphane-3,5-dione (Example 112);
(S,E)-11-methyl-1⁷-(2-oxopyrrolidin-1-yl)-1²,1³-dihydro-1¹H-2,6-diaza-1(2,1)-benzo[d]imidazola-4(1,3)-benzenacycloundecaphane-3,5-dione (Example 113);
(S,E)-11-methyl-1⁷-(2-oxa-6-azaspiro[3.3]heptane-6-carbonyl)-1²,1³-dihydro-1¹H-2,6-diaza-1(2,1)-benzo[d]imidazola-4(1,3)-benzenacycloundecaphane-3,5-dione (Example 114);
(S,E)-11-methyl-1⁷-(2-oxa-6-azaspiro[3.4]octane-6-carbonyl)-1²,1³-dihydro-1¹H-2,6-diaza-1(2,1)-benzo[d]imidazola-4(1,3)-benzenacycloundecaphane-3,5-dione (Example 115);
(S,E)-11-methyl-1⁷-(5-oxa-2-azaspiro[3.4]octane-2-carbonyl)-1²,1³-dihydro-1¹H-2,6-diaza-1(2,1)-benzo[d]imidazola-4(1,3)-benzenacycloundecaphane-3,5-dione (Example 116);
(S,E)-1⁷-(7,7-difluoro-5-oxa-2-azaspiro[3.4]octane-2-carbonyl)-11-methyl-1²,1³-dihydro-1¹H-2,6-diaza-1(2,1)-benzo[d]imidazola-4(1,3)-benzenacycloundecaphane-3,5-dione (Example 117);
(S,E)-11-methyl-N,N-bis(methyl-d₃)-3,5-dioxo-1²,1³-dihydro-1¹H-2,6-diaza-1(2,1)-benzo[d]imidazola-4(1,3)-benzenacycloundecaphane-1⁷-carboxamide (Example 118);
(S,E)-11-methyl-1⁴-(2-oxopyrrolidin-1-yl)-1²,1³-dihydro-1¹H-2,6-diaza-1(2,3)-imidazo[4,5-c]pyridina-4(1,3)-benzenacycloundecaphane-3,5-dione (Example 119);
(11S,E)-11-methyl-1⁷-(1-oxa-6-azaspiro[3.4]octane-6-carbonyl)-1²,1³-dihydro-1¹H-2,6-diaza-1(2,1)-benzo[d]imidazola-4(1,3)-benzenacycloundecaphane-3,5-dione (Examples 120 and 121);
(S,E)-1⁴-(3-cyclopropyl-2-oxoimidazolidin-1-yl)-11-methyl-1²,1³-dihydro-1¹H-2,6-diaza-1(2,3)-imidazo[4,5-c]pyridina-4(1,3)-benzenacycloundecaphane-3,5-dione (Example 122);
(S,E)-1⁵-(2-hydroxypropan-2-yl)-N,N,11-trimethyl-3,5-dioxo-1²,1³-dihydro-1¹H-2,6-diaza-1(2,1)-benzo[d]imidazola-4(1,3)-benzenacycloundecaphane-1⁷-carboxamide (Example 123);
(S,E)-N,N,11-trimethyl-1⁵-((4-methylpiperazin-1-yl)methyl)-3,5-dioxo-1²,1³-dihydro-1¹H-2,6-diaza-1(2,1)-benzo[d]imidazola-4(1,3)-benzenacycloundecaphane-1⁷-carboxamide (Example 124);
(S,E)-N,N,11-trimethyl-1⁵-(morpholinomethyl)-3,5-dioxo-1²,1³-dihydro-1¹H-2,6-diaza-1(2,1)-benzo[d]imidazola-4(1,3)-benzenacycloundecaphane-1⁷-carboxamide (Example 125);
(11S,E)-1⁴-(3-fluoro-2-oxopyrrolidin-1-yl)-11-methyl-1²,1³-dihydro-1¹H-2,6-diaza-1(2,3)-imidazo[4,5-c]pyridina-4(1,3)-benzenacycloundecaphane-3,5-dione (Examples 126 and 127);
(S,E)-N,N,11-trimethyl-3,5-dioxo-1²,1³-dihydro-1¹H-2,6-diaza-1(2,3)-imidazo[4,5-c]pyridina-4(1,3)-benzenacycloundecaphane-1⁴-carboxamide (Example 128);
(S,E)-11-methyl-1⁷-(1-(oxetan-3-yl)-1H-1,2,3-triazol-4-yl)-1²,1³-dihydro-1¹H-2,6-diaza-1(2,1)-benzo[d]imidazola-4(1,3)-benzenacycloundecaphane-3,5-dione (Example 129);
(S,E)-11-methyl-1⁴-(3-methyl-2-oxoimidazolidin-1-yl)-1²,1³-dihydro-1¹H-2,6-diaza-1(2,3)-imidazo[4,5-c]pyridina-4(1,3)-benzenacycloundecaphane-3,5-dione (Example 130);
(E)-N,N-dimethyl-3′,5′-dioxospiro[cyclopropane-1,11′-2,6-diaza-1(2,1)-benzo[d]imidazola-4(1,3)-benzenacycloundecaphane]-7′-carboxamide (Example 131);
(E)-11-methyl-1⁷-(5-methyl-1,3,4-oxadiazol-2-yl)-1²,1³-dihydro-1¹H-2,6-diaza-1(2,1)-benzo[d]imidazola-4(1,3)-benzenacycloundecaphane-3,5-dione (Examples 132 and 133);
(S,E)-1⁷-(4-isopropyl-5-methyl-4H-1,2,4-triazol-3-yl)-11-methyl-1²,1³-dihydro-1¹H-2,6-diaza-1(2,1)-benzo[d]imidazola-4(1,3)-benzenacycloundecaphane-3,5-dione (Example 134);
(E)-11-methyl-1⁷-(1-methyl-1H-imidazol-2-yl)-1²,1³-dihydro-1¹H-2,6-diaza-1(2,1)-benzo[d]imidazola-4(1,3)-benzenacycloundecaphane-3,5-dione (Examples 135 and 136);
(E)-1⁷-(1-(difluoromethyl)-1H-1,2,4-triazol-3-yl)-11-methyl-1²,1³-dihydro-1¹H-2,6-diaza-1(2,1)-benzo[d]imidazola-4(1,3)-benzenacycloundecaphane-3,5-dione (Examples 137 and 138);
(E)-1⁷-(1-(difluoromethyl)-1H-1,2,4-triazol-5-yl)-11-methyl-1²,1³-dihydro-1¹H-2,6-diaza-1(2,1)-benzo[d]imidazola-4(1,3)-benzenacycloundecaphane-3,5-dione (Examples 139 and 140);
(E)-1⁷-(3-cyclopropyl-2-oxoimidazolidin-1-yl)-11-methyl-1²,1³-dihydro-1¹H-2,6-diaza-1(2,1)-benzo[d]imidazola-4(1,3)-benzenacycloundecaphane-3,5-dione (Examples 141 and 142);
(E)-11-methyl-1⁷-(3-(oxetan-3-yl)-2-oxoimidazolidin-1-yl)-1²,1³-dihydro-1¹H-2,6-diaza-1(2,1)-benzo[d]imidazola-4(1,3)-benzenacycloundecaphane-3,5-dione (Examples 143 and 144);
(S,E)-N,N,11-trimethyl-3,5-dioxo-1²,1³-dihydro-1¹H-2,6-diaza-1(2,1)-imidazo[4,5-c]pyridina-4(1,3)-benzenacycloundecaphane-1⁷-carboxamide (Example 145);
(11S,E)-1⁷-(3-fluoro-2-oxopyrrolidin-1-yl)-11-methyl-1²,1³-dihydro-1¹H-2,6-diaza-1(2,1)-benzo[d]imidazola-4(1,3)-benzenacycloundecaphane-3,5-dione (Example 146); and
(S,E)-11-methyl-1⁷-(3-methyl-1,2,4-oxadiazol-5-yl)-1²,1³-dihydro-1¹H-2,6-diaza-1(2,1)-benzo[d]imidazola-4(1,3)-benzenacycloundecaphane-3,5-dione (Example 147)
or a pharmaceutically acceptable salt or solvate thereof.

19. A pharmaceutical composition comprising a compound as claimed in any one of claims 1 to 18 and at least one pharmaceutically acceptable excipient.

20. A compound as claimed in any one of claims 1 to 18, or a composition as claimed in claim 19, for use as a medicament.

21. A compound as claimed in any one of claims 1 to 18, or a composition as claimed in claim 19, for use in the treatment or prophylaxis of a condition selected from cancer, inflammatory diseases, autoinflammatory diseases and autoimmune diseases.

22. A compound as claimed in any one of claims 1 to 18, or a composition as claimed in claim 19, for use in the treatment or prophylaxis of a disease or disorder that may be treated or prevented by inhibition of TAK1.

23. Use of a compound as claimed in any one of claims 1 to 18 in the manufacture of a medicament to be used in the treatment or prophylaxis of a condition selected from cancer, inflammatory diseases, autoinflammatory diseases and autoimmune diseases.

24. A method of treatment or prophylaxis comprising a step of administering a compound as claimed in any one of claims 1 to 18, or a composition as claimed in claim 19, to a subject in need thereof.

25. A method as claimed in claim 24, wherein the subject has a condition selected from cancer, inflammatory diseases, autoinflammatory diseases and autoimmune diseases.

26. A compound for use as claimed in claim 21, a use as claimed in claim 23, or a method as claimed in claim 25, wherein the inflammatory disease or autoimmune disease is systemic lupus erythematosus (SLE), Sjögren's syndrome, rheumatoid arthritis, osteoarthritis, gout, psoriatic arthritis, psoriasis, ankylosing spondylitis, diabetes mellitus, malaria, Crohn's disease, ulcerative colitis, inflammatory bowel disease, Familial Mediterranean Fever (FMF), Cryopyrin-associated periodic syndromes (CAPS), Deficiency of IL-1-Receptor Antagonist (DIRA) and Hyper IgD Syndrome (HIDS), myocardial infarction, reperfusion, ischemia, stroke, multiple sclerosis or systemic sclerosis.

27. A compound for use as claimed in claim 21, a use as claimed in claim 23, or a method as claimed in claim 25, wherein the cancer is one that is associated with a mutation of the KRAS gene, for example the cancer is one classed as a KRAS-dependent cancer.

28. A compound for use as claimed in claim 21, a use as claimed in claim 23, or a method as claimed in claim 25, wherein the cancer is a carcinoma, a sarcoma, a myeloma, a leukemia or a lymphoma.

29. A compound as claimed in any one of claims 1 to 18, or a composition as claimed in claim 19, for use in the treatment or prophylaxis of a condition selected from idiopathic pulmonary fibrosis, asthma and chronic obstructive pulmonary disease (COPD).
